SEQUENCE LISTING (1635 sequences)

SEQ ID NO: 1 - protein, 718 aa, synthetic construct
VPDKKETPLVALCKKSFPGLRFKKHDSRQAGRILKSKGEGAAVAFLEGKGGTTQPNFKPPVKCNIVAMSRPLEEWPIYKASVVIQKYVYAQSYEEFKATDPGKSEAGLRAWLKATRVDTDGYFNVQGLNLIFQNARATYEGVLKKVENRNSKKVAKIEQRNEHRAERGLPLLTLDEPETALDETGHLRHRPGINCSVFGYQHMKLKPYVPGSIPGVTGYSRDPSTPIAACGVDRLEIPEGQPGYVPPWDRENLSVKKHRRKRASWARSRGGAIDDNMLLAVVRVADDWALLDLRGLLRNTQYRKLLDRSVPVTIESLLNLVTNDPTLSVVKKPGKPVRYTATLIYKQGVVPVVKAKVVKGSYVSKMLDDTTETFSLVGVDLGVNNLIAANALRIRPGKCVERLQAFTLPEQTVEDFFRFRKAYDKHQENLRLAAVRSLTAEQQAEVLALDTFGPEQAKMQVCGHLGLSVDEVPWDKVNSRSSILSDLAKERGVDDTLYMFPFFKGKGKKRKTEIRKRWDVNWAQHFRPQLTSETRKALNEAKWEAERNSSKYHQLSIRKKELSRHCVNYVIRTAEKRAQCGKVIVAVEDLHHSFRRGGKGSRKSGWGGFFAAKQEGRWLMDALFGAFCDLAVHRGYRVIKVDPYNTSRTCPECGHCDKANRDRVNREAFICVCCGYRGNADIDVAAYNIAMVAITGVSLRKAARASVASTPLESLAAE

SEQ ID NO: 2 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 3 - protein, 468 aa, synthetic construct
MPTITRKIELTLLTEGLSEEQRKEQWGLLYHINDNLYKAANNISSKLYLDDHVSSMVRMKHAEYLSLLKELARAEKQKTPDADAIAELRKKVAAAEKEMTDQEHAICKYATEMSTQSLSYRFATELETNIFAKILDCLKQGVFATFNSDARDVKRGERAIRNYKKGMPIPFAWDKSLRIEKDNKDFYLRWYNGLRFLFNFGKDRSNNRLIVERCLKMDADYDGEYKLCNSSIQIAKREGKTKLFLLLVVKIPQEHVELNKKVVVGVDLGINVPAYVATNITEERKAIGDREHFLNSRMAFQRRYKSLQRLRGTAGGKGRAKKLEPLERLRKAEHNWVHTQNHLFSREVVDFAVKSHAATIHMEDLSGFGKDNDGNADERKEFVLRNWSYYELQNMIAYKAAKYGIKVEKIHPAYTSKTCSWCGQLGFREGVTFICENPECKQCGEKVHADYNAARNIANSKDIIKKNE

SEQ ID NO: 4 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 5-6 - skipped (2 entries)

SEQ ID NO: 7 - protein, 8 aa, synthetic construct
  VARIANT 3: note = Any naturally occurring amino acid
  VARIANT 4: note = W, F, or Y
  VARIANT 5: note = Any naturally occurring amino acid
  VARIANT 6: note = Any naturally occurring amino acid
KRXXXXAF

SEQ ID NO: 8 - skipped

SEQ ID NO: 9 - protein, 6 aa, synthetic construct
  VARIANT 5: note = K or R
  VARIANT 6: note = W, F, or Y
LGKRXX

SEQ ID NO: 10 - skipped

SEQ ID NO: 11 - protein, 20 aa, synthetic construct
  VARIANT 4: note = Any naturally occurring amino acid
  VARIANT 8: note = Any naturally occurring amino acid
  VARIANT 11: note = Any naturally occurring amino acid
  VARIANT 15: note = Any naturally occurring amino acid
  VARIANT 19: note = Any naturally occurring amino acid
GLFXALLXLLXSLWXLLLXA

SEQ ID NO: 12-17 - skipped (6 entries)

SEQ ID NO: 18 - protein, 16 aa, synthetic construct
APKKKRKVGIHGVPAA

SEQ ID NO: 19 - protein, 20 aa, synthetic construct
  VARIANT 4: note = K, H, or R
  VARIANT 8: note = K, H, or R
  VARIANT 11: note = K, H, or R
  VARIANT 15: note = K, H, or R
  VARIANT 19: note = K, H, or R
GLFXALLXLLXSLWXLLLXA

SEQ ID NO: 20 - protein, 20 aa, synthetic construct
GLFHALLHLLHSLWHLLLHA

SEQ ID NO: 21 - protein, 15 aa, synthetic construct
PKKKRKVGIHGVPAA

SEQ ID NO: 22 - protein, 16 aa, synthetic construct
KRPAATKKAGQAKKKK

SEQ ID NO: 23 - protein, 23 aa, synthetic construct
MDYKDHDGDYKDHDIDYKDDDDK

SEQ ID NO: 24 - protein, 9 aa, synthetic construct
MAPKKKRKV

SEQ ID NO: 25 - protein, 8 aa, synthetic construct
GIHGVPAA

SEQ ID NO: 26 - protein, 23 aa, synthetic construct
EFGSGEGRGSLLTCGDVEENPGP

SEQ ID NO: 27 - protein, 132 aa, synthetic construct
MAKPLSQEESTLIERATATINSIPISEDYSVASAALSSDGRIFTGVNVYHFTGGPCAELVVLGTAAAAAAGNLTCIVAIGNENRGILSPCGRCRQVLLDLHPGIKAIVKDSDGQPTAVGIRELLPSGYVWEG

SEQ ID NO: 28 - RNA, 20 nt, synthetic construct
  source 1..20: mol_type = other RNA
tactgacaaagatatcactc

SEQ ID NO: 29 - RNA, 20 nt, synthetic construct
  source 1..20: mol_type = other RNA
cctgcatttgcagagtttag

SEQ ID NO: 30 - RNA, 20 nt, synthetic construct
  source 1..20: mol_type = other RNA
cttgaaactaaactctgcaa

SEQ ID NO: 31 - RNA, 20 nt, synthetic construct
  source 1..20: mol_type = other RNA
tacataatccatctattttt

SEQ ID NO: 32 - RNA, 20 nt, synthetic construct
  source 1..20: mol_type = other RNA
tgaagggtgatggaaattac

SEQ ID NO: 33 - RNA, 20 nt, synthetic construct
  source 1..20: mol_type = other RNA
aagagtccagatgtgctgaa

SEQ ID NO: 34 - RNA, 20 nt, synthetic construct
  source 1..20: mol_type = other RNA
tcatcattatattactagaa

SEQ ID NO: 35 - RNA, 20 nt, synthetic construct
  source 1..20: mol_type = other RNA
tttcgaaattgtatttatct

SEQ ID NO: 36 - RNA, 20 nt, synthetic construct
  source 1..20: mol_type = other RNA
ttgtcatcattatattacta

SEQ ID NO: 37 - RNA, 20 nt, synthetic construct
  source 1..20: mol_type = other RNA
catagggaaattttcacatg

SEQ ID NO: 38 - RNA, 20 nt, synthetic construct
  source 1..20: mol_type = other RNA
ctgtaagataccaaaaaggc

SEQ ID NO: 39 - RNA, 20 nt, synthetic construct
  source 1..20: mol_type = other RNA
tgcctttttggtatcttaca

SEQ ID NO: 40 - RNA, 20 nt, synthetic construct
  source 1..20: mol_type = other RNA
tactggcaaagaaagaaata

SEQ ID NO: 41 - RNA, 20 nt, synthetic construct
  source 1..20: mol_type = other RNA
aatcttttctcaaataaaaa

SEQ ID NO: 42 - RNA, 20 nt, synthetic construct
  source 1..20: mol_type = other RNA
attaatggttgataggttct

SEQ ID NO: 43 - RNA, 20 nt, synthetic construct
  source 1..20: mol_type = other RNA
gtgcctttcaccctgcttat

SEQ ID NO: 44 - RNA, 20 nt, synthetic construct
  source 1..20: mol_type = other RNA
tcattcctattagatctgtc

SEQ ID NO: 45 - RNA, 20 nt, synthetic construct
  source 1..20: mol_type = other RNA
ataggttctttaatgttagt

SEQ ID NO: 46 - RNA, 20 nt, synthetic construct
  source 1..20: mol_type = other RNA
tttaatgttagtgcctttca

SEQ ID NO: 47 - RNA, 20 nt, synthetic construct
  source 1..20: mol_type = other RNA
atcgaataagtaatgtgtat

SEQ ID NO: 48 - RNA, 20 nt, synthetic construct
  source 1..20: mol_type = other RNA
gaagatctgagctctgagtg

SEQ ID NO: 49 - RNA, 20 nt, synthetic construct
  source 1..20: mol_type = other RNA
aagaggaagttagaagatct

SEQ ID NO: 50 - RNA, 20 nt, synthetic construct
  source 1..20: mol_type = other RNA
atgaattatcttcaaagtgt

SEQ ID NO: 51 - RNA, 20 nt, synthetic construct
  source 1..20: mol_type = other RNA
atttagaagtaaaatataat

SEQ ID NO: 52 - RNA, 20 nt, synthetic construct
  source 1..20: mol_type = other RNA
aaaagtgcaactatgaagtg

SEQ ID NO: 53 - RNA, 20 nt, synthetic construct
  source 1..20: mol_type = other RNA
atctctatctttagaattga

SEQ ID NO: 54 - RNA, 20 nt, synthetic construct
  source 1..20: mol_type = other RNA
caattctaaagatagagata

SEQ ID NO: 55 - RNA, 20 nt, synthetic construct
  source 1..20: mol_type = other RNA
cacttttgaacaaatagcta

SEQ ID NO: 56 - RNA, 20 nt, synthetic construct
  source 1..20: mol_type = other RNA
ttgactgtgcaaaaagtctt

SEQ ID NO: 57 - RNA, 20 nt, synthetic construct
  source 1..20: mol_type = other RNA
ggctcaaattgttactcttc

SEQ ID NO: 58 - RNA, 20 nt, synthetic construct
  source 1..20: mol_type = other RNA
ttgaaattggctctttagct

SEQ ID NO: 59 - RNA, 20 nt, synthetic construct
  source 1..20: mol_type = other RNA
ctcttcaattaaatttgact

SEQ ID NO: 60 - RNA, 20 nt, synthetic construct
  source 1..20: mol_type = other RNA
ttgcaaaaaggaaaaaagaa

SEQ ID NO: 61 - RNA, 20 nt, synthetic construct
  source 1..20: mol_type = other RNA
ctaatttttctttttcttct

SEQ ID NO: 62 - RNA, 20 nt, synthetic construct
  source 1..20: mol_type = other RNA
gctgagagagaaacagttgc

SEQ ID NO: 63 - RNA, 20 nt, synthetic construct
  source 1..20: mol_type = other RNA
ctctctcagcaaacacatta

SEQ ID NO: 64 - RNA, 20 nt, synthetic construct
  source 1..20: mol_type = other RNA
cctaagaactggtgggaaat

SEQ ID NO: 65 - RNA, 20 nt, synthetic construct
  source 1..20: mol_type = other RNA
ttaggcaactgtttctctct

SEQ ID NO: 66 - RNA, 20 nt, synthetic construct
  source 1..20: mol_type = other RNA
gcagaagtttttctttaaaa

SEQ ID NO: 67 - RNA, 20 nt, synthetic construct
  source 1..20: mol_type = other RNA
ttctttaaaatgaagatttt

SEQ ID NO: 68 - RNA, 20 nt, synthetic construct
  source 1..20: mol_type = other RNA
atcatcctagccataacaca

SEQ ID NO: 69 - RNA, 20 nt, synthetic construct
  source 1..20: mol_type = other RNA
ttcatcatcctagccataac

SEQ ID NO: 70 - RNA, 20 nt, synthetic construct
  source 1..20: mol_type = other RNA
aaagaattcagaatcagtgg

SEQ ID NO: 71 - RNA, 20 nt, synthetic construct
  source 1..20: mol_type = other RNA
ggaagttacatttaaaatgt

SEQ ID NO: 72 - RNA, 20 nt, synthetic construct
  source 1..20: mol_type = other RNA
catttaaaatgtctcctcca

SEQ ID NO: 73 - RNA, 20 nt, synthetic construct
  source 1..20: mol_type = other RNA
tctcacatttatgttgctta

SEQ ID NO: 74 - RNA, 20 nt, synthetic construct
  source 1..20: mol_type = other RNA
cttatttaaaaaattattca

SEQ ID NO: 75 - RNA, 20 nt, synthetic construct
  source 1..20: mol_type = other RNA
ttaacagaaaatcacgttaa

SEQ ID NO: 76 - RNA, 20 nt, synthetic construct
  source 1..20: mol_type = other RNA
aagctgaaatgaacagtaga

SEQ ID NO: 77 - RNA, 20 nt, synthetic construct
  source 1..20: mol_type = other RNA
atttcagctttaacgtgatt

SEQ ID NO: 78 - RNA, 20 nt, synthetic construct
  source 1..20: mol_type = other RNA
ataactttacattaaacatc

SEQ ID NO: 79 - RNA, 20 nt, synthetic construct
  source 1..20: mol_type = other RNA
gtatcaaagataccttttta

SEQ ID NO: 80 - RNA, 20 nt, synthetic construct
  source 1..20: mol_type = other RNA
catttgataaaattatacca

SEQ ID NO: 81 - RNA, 20 nt, synthetic construct
  source 1..20: mol_type = other RNA
aatgtaaagttattaacaga

SEQ ID NO: 82 - RNA, 20 nt, synthetic construct
  source 1..20: mol_type = other RNA
ctgtgattttcttttggatt

SEQ ID NO: 83 - RNA, 20 nt, synthetic construct
  source 1..20: mol_type = other RNA
aatcctttaacatttcattc

SEQ ID NO: 84 - RNA, 20 nt, synthetic construct
  source 1..20: mol_type = other RNA
tgaaggtgttcttgtacttc

SEQ ID NO: 85 - RNA, 20 nt, synthetic construct
  source 1..20: mol_type = other RNA
ttgtacttcatcccactgat

SEQ ID NO: 86 - RNA, 20 nt, synthetic construct
  source 1..20: mol_type = other RNA
cctccggttctgaaggtgtt

SEQ ID NO: 87 - RNA, 20 nt, synthetic construct
  source 1..20: mol_type = other RNA
aaggattcaacacaatggct

SEQ ID NO: 88 - RNA, 20 nt, synthetic construct
  source 1..20: mol_type = other RNA
aatgaaatgttaaaggattc

SEQ ID NO: 89 - RNA, 20 nt, synthetic construct
  source 1..20: mol_type = other RNA
tgtcaccagagtaacagtct

SEQ ID NO: 90 - RNA, 20 nt, synthetic construct
  source 1..20: mol_type = other RNA
tgtccaagcccggttgaaat

SEQ ID NO: 91 - RNA, 20 nt, synthetic construct
  source 1..20: mol_type = other RNA
ggagatggcagtttccttag

SEQ ID NO: 92 - RNA, 20 nt, synthetic construct
  source 1..20: mol_type = other RNA
ccttagtaaccacaggttgt

SEQ ID NO: 93 - RNA, 20 nt, synthetic construct
  source 1..20: mol_type = other RNA
aaatctgccagagcaggtac

SEQ ID NO: 94 - RNA, 20 nt, synthetic construct
  source 1..20: mol_type = other RNA
atatcctcaaggtcacccac

SEQ ID NO: 95 - RNA, 20 nt, synthetic construct
  source 1..20: mol_type = other RNA
taaaatcacagagggtgatg

SEQ ID NO: 96 - RNA, 20 nt, synthetic construct
  source 1..20: mol_type = other RNA
gaggtacctgctctggcaga

SEQ ID NO: 97 - RNA, 20 nt, synthetic construct
  source 1..20: mol_type = other RNA
ctctggtgacacaacctgtg

SEQ ID NO: 98 - RNA, 20 nt, synthetic construct
  source 1..20: mol_type = other RNA
ctaaggaaactgccatctcc

SEQ ID NO: 99 - RNA, 20 nt, synthetic construct
  source 1..20: mol_type = other RNA
accgccttccactcagagct

SEQ ID NO: 100 - RNA, 20 nt, synthetic construct
  source 1..20: mol_type = other RNA
acttcaagagctgagggcaa

SEQ ID NO: 101 - RNA, 20 nt, synthetic construct
  source 1..20: mol_type = other RNA
ttgaggattgctgaattatt

SEQ ID NO: 102 - RNA, 20 nt, synthetic construct
  source 1..20: mol_type = other RNA
gcagacctcctgccaccgca

SEQ ID NO: 103 - RNA, 20 nt, synthetic construct
  source 1..20: mol_type = other RNA
cattcaatgttctgacaaca

SEQ ID NO: 104 - RNA, 20 nt, synthetic construct
  source 1..20: mol_type = other RNA
tgacaacagtttgccgctgc

SEQ ID NO: 105 - RNA, 20 nt, synthetic construct
  source 1..20: mol_type = other RNA
gccgctgcccaatgccatcc

SEQ ID NO: 106 - RNA, 20 nt, synthetic construct
  source 1..20: mol_type = other RNA
tcagaacattgaatgcaact

SEQ ID NO: 107 - RNA, 20 nt, synthetic construct
  source 1..20: mol_type = other RNA
ccaattctcaggaatttgtg

SEQ ID NO: 108 - RNA, 20 nt, synthetic construct
  source 1..20: mol_type = other RNA
agcttctgttagccactgat

SEQ ID NO: 109 - RNA, 20 nt, synthetic construct
  source 1..20: mol_type = other RNA
gccactgattaaatatcttt

SEQ ID NO: 110 - RNA, 20 nt, synthetic construct
  source 1..20: mol_type = other RNA
ctcagaaagacacaaattcc

SEQ ID NO: 111 - RNA, 20 nt, synthetic construct
  source 1..20: mol_type = other RNA
agaaatggcggcgttttcat

SEQ ID NO: 112 - RNA, 17 nt, synthetic construct
  source 1..17: mol_type = other RNA
cctgcaggcgatttgac

SEQ ID NO: 113 - RNA, 17 nt, synthetic construct
  source 1..17: mol_type = other RNA
acctgcaggcgatttga

SEQ ID NO: 114 - RNA, 17 nt, synthetic construct
  source 1..17: mol_type = other RNA
tacctgcaggcgatttg

SEQ ID NO: 115 - RNA, 17 nt, synthetic construct
  source 1..17: mol_type = other RNA
tagagatatagcgtata

SEQ ID NO: 116 - RNA, 17 nt, synthetic construct
  source 1..17: mol_type = other RNA
ggttatactgacaaaga

SEQ ID NO: 117 - RNA, 17 nt, synthetic construct
  source 1..17: mol_type = other RNA
gacagatctgttgagaa

SEQ ID NO: 118 - RNA, 17 nt, synthetic construct
  source 1..17: mol_type = other RNA
ctcaacagatctgtcaa

SEQ ID NO: 119 - RNA, 17 nt, synthetic construct
  source 1..17: mol_type = other RNA
atccatatgcttttacc

SEQ ID NO: 120 - RNA, 17 nt, synthetic construct
  source 1..17: mol_type = other RNA
gttacttgaaactaaac

SEQ ID NO: 121 - RNA, 17 nt, synthetic construct
  source 1..17: mol_type = other RNA
gcagagtttagtttcaa

SEQ ID NO: 122 - RNA, 17 nt, synthetic construct
  source 1..17: mol_type = other RNA
gttatactgacaaagat

SEQ ID NO: 123 - RNA, 17 nt, synthetic construct
  source 1..17: mol_type = other RNA
tttggttatactgacaa

SEQ ID NO: 124 - RNA, 17 nt, synthetic construct
  source 1..17: mol_type = other RNA
cctgcatttgcagagtt

SEQ ID NO: 125 - RNA, 17 nt, synthetic construct
  source 1..17: mol_type = other RNA
cttgaaactaaactctg

SEQ ID NO: 126 - RNA, 17 nt, synthetic construct
  source 1..17: mol_type = other RNA
ttcttgatccatatgct

SEQ ID NO: 127 - RNA, 17 nt, synthetic construct
  source 1..17: mol_type = other RNA
cttgatccatatgcttt

SEQ ID NO: 128 - RNA, 17 nt, synthetic construct
  source 1..17: mol_type = other RNA
tcttgatccatatgctt

SEQ ID NO: 129 - RNA, 17 nt, synthetic construct
  source 1..17: mol_type = other RNA
tactgacaaagatatca

SEQ ID NO: 130 - RNA, 17 nt, synthetic construct
  source 1..17: mol_type = other RNA
gtcagtataaccaaaaa

SEQ ID NO: 131 - RNA, 17 nt, synthetic construct
  source 1..17: mol_type = other RNA
ctgcatttgcagagttt

SEQ ID NO: 132 - RNA, 17 nt, synthetic construct
  source 1..17: mol_type = other RNA
tacataatccatctatt

SEQ ID NO: 133 - RNA, 17 nt, synthetic construct
  source 1..17: mol_type = other RNA
aaactaaactctgcaaa

SEQ ID NO: 134 - RNA, 17 nt, synthetic construct
  source 1..17: mol_type = other RNA
acagatctgttgagaaa

SEQ ID NO: 135 - RNA, 17 nt, synthetic construct
  source 1..17: mol_type = other RNA
gtttcaagtaacaaata

SEQ ID NO: 136 - RNA, 17 nt, synthetic construct
  source 1..17: mol_type = other RNA
tgtaaaacagattatag

SEQ ID NO: 137 - RNA, 17 nt, synthetic construct
  source 1..17: mol_type = other RNA
ttacttgaaactaaact

SEQ ID NO: 138 - RNA, 17 nt, synthetic construct
  source 1..17: mol_type = other RNA
acataatccatctattt

SEQ ID NO: 139 - RNA, 17 nt, synthetic construct
  source 1..17: mol_type = other RNA
cataatccatctatttt

SEQ ID NO: 140 - RNA, 17 nt, synthetic construct
  source 1..17: mol_type = other RNA
tcagtataaccaaaaaa

SEQ ID NO: 141 - RNA, 17 nt, synthetic construct
  source 1..17: mol_type = other RNA
ttgatccatatgctttt

SEQ ID NO: 142 - RNA, 17 nt, synthetic construct
  source 1..17: mol_type = other RNA
catcacccttcagaacc

SEQ ID NO: 143 - RNA, 17 nt, synthetic construct
  source 1..17: mol_type = other RNA
agcacatctggactctt

SEQ ID NO: 144 - RNA, 17 nt, synthetic construct
  source 1..17: mol_type = other RNA
aagagtccagatgtgct

SEQ ID NO: 145 - RNA, 17 nt, synthetic construct
  source 1..17: mol_type = other RNA
atcttcagcacatctgg

SEQ ID NO: 146 - RNA, 17 nt, synthetic construct
  source 1..17: mol_type = other RNA
gactgttgttgtcatca

SEQ ID NO: 147 - RNA, 17 nt, synthetic construct
  source 1..17: mol_type = other RNA
aagatcaggttctgaag

SEQ ID NO: 148 - RNA, 17 nt, synthetic construct
  source 1..17: mol_type = other RNA
agaagttaaagagtcca

SEQ ID NO: 149 - RNA, 17 nt, synthetic construct
  source 1..17: mol_type = other RNA
aagaagttaaagagtcc

SEQ ID NO: 150 - RNA, 17 nt, synthetic construct
  source 1..17: mol_type = other RNA
ccttaagataccatttg

SEQ ID NO: 151 - RNA, 17 nt, synthetic construct
  source 1..17: mol_type = other RNA
tcttcagcacatctgga

SEQ ID NO: 152 - RNA, 17 nt, synthetic construct
  source 1..17: mol_type = other RNA
tgaagggtgatggaaat

SEQ ID NO: 153 - RNA, 17 nt, synthetic construct
  source 1..17: mol_type = other RNA
acttcttaaagatcagg

SEQ ID NO: 154 - RNA, 17 nt, synthetic construct
  source 1..17: mol_type = other RNA
tgactgttgttgtcatc

SEQ ID NO: 155 - RNA, 17 nt, synthetic construct
  source 1..17: mol_type = other RNA
actgttgttgtcatcat

SEQ ID NO: 156 - RNA, 17 nt, synthetic construct
  source 1..17: mol_type = other RNA
tcgaaattgtatttatc

SEQ ID NO: 157 - RNA, 17 nt, synthetic construct
  source 1..17: mol_type = other RNA
ctagtaatataatgatg

SEQ ID NO: 158 - RNA, 17 nt, synthetic construct
  source 1..17: mol_type = other RNA
aggtaagtctttgattt

SEQ ID NO: 159 - RNA, 17 nt, synthetic construct
  source 1..17: mol_type = other RNA
ttaaagatcaggttctg

SEQ ID NO: 160 - RNA, 17 nt, synthetic construct
  source 1..17: mol_type = other RNA
cgaaattgtatttatct

SEQ ID NO: 161 - RNA, 17 nt, synthetic construct
  source 1..17: mol_type = other RNA
aacttcttaaagatcag

SEQ ID NO: 162 - RNA, 17 nt, synthetic construct
  source 1..17: mol_type = other RNA
gttttttcgaaattgta

SEQ ID NO: 163 - RNA, 17 nt, synthetic construct
  source 1..17: mol_type = other RNA
tatttatcttcagcaca

SEQ ID NO: 164 - RNA, 17 nt, synthetic construct
  source 1..17: mol_type = other RNA
tagtaatataatgatga

SEQ ID NO: 165 - RNA, 17 nt, synthetic construct
  source 1..17: mol_type = other RNA
tcatcattatattacta

SEQ ID NO: 166 - RNA, 17 nt, synthetic construct
  source 1..17: mol_type = other RNA
agataccatttgtattt

SEQ ID NO: 167 - RNA, 17 nt, synthetic construct
  source 1..17: mol_type = other RNA
ctagaaagaaaattatc

SEQ ID NO: 168 - RNA, 17 nt, synthetic construct
  source 1..17: mol_type = other RNA
gaaattgtatttatctt

SEQ ID NO: 169 - RNA, 17 nt, synthetic construct
  source 1..17: mol_type = other RNA
tctttctagtaatataa

SEQ ID NO: 170 - RNA, 17 nt, synthetic construct
  source 1..17: mol_type = other RNA
ttcgaaattgtatttat

SEQ ID NO: 171 - RNA, 17 nt, synthetic construct
  source 1..17: mol_type = other RNA
tttcgaaattgtattta

SEQ ID NO: 172 - RNA, 17 nt, synthetic construct
  source 1..17: mol_type = other RNA
ttgtcatcattatatta

SEQ ID NO: 173 - RNA, 17 nt, synthetic construct
  source 1..17: mol_type = other RNA
tttctagtaatataatg

SEQ ID NO: 174 - RNA, 17 nt, synthetic construct
  source 1..17: mol_type = other RNA
tattactagaaagaaaa

SEQ ID NO: 175 - RNA, 17 nt, synthetic construct
  source 1..17: mol_type = other RNA
gatttgttttttcgaaa

SEQ ID NO: 176 - RNA, 17 nt, synthetic construct
  source 1..17: mol_type = other RNA
ttttttcgaaattgtat

SEQ ID NO: 177 - RNA, 17 nt, synthetic construct
  source 1..17: mol_type = other RNA
cttttgactgttgttgt

SEQ ID NO: 178 - RNA, 17 nt, synthetic construct
  source 1..17: mol_type = other RNA
atttgttttttcgaaat

SEQ ID NO: 179 - RNA, 17 nt, synthetic construct
  source 1..17: mol_type = other RNA
gccagtacaactgcatg

SEQ ID NO: 180 - RNA, 17 nt, synthetic construct
  source 1..17: mol_type = other RNA
aacagtgtgctaccaca

SEQ ID NO: 181 - RNA, 17 nt, synthetic construct
  source 1..17: mol_type = other RNA
ggtatcttacaggaact

SEQ ID NO: 182 - RNA, 17 nt, synthetic construct
  source 1..17: mol_type = other RNA
tggtatcttacaggaac

SEQ ID NO: 183 - RNA, 17 nt, synthetic construct
  source 1..17: mol_type = other RNA
gtatcttacaggaactc

SEQ ID NO: 184 - RNA, 17 nt, synthetic construct
  source 1..17: mol_type = other RNA
ccagtacaactgcatgt

SEQ ID NO: 185 - RNA, 17 nt, synthetic construct
  source 1..17: mol_type = other RNA
tttgccagtacaactgc

SEQ ID NO: 186 - RNA, 17 nt, synthetic construct
  source 1..17: mol_type = other RNA
caggaactccaggatgg

SEQ ID NO: 187 - RNA, 17 nt, synthetic construct
  source 1..17: mol_type = other RNA
ctttgccagtacaactg

SEQ ID NO: 188 - RNA, 17 nt, synthetic construct
  source 1..17: mol_type = other RNA
cacatggagcttttgta

SEQ ID NO: 189 - RNA, 17 nt, synthetic construct
  source 1..17: mol_type = other RNA
ccctatgaaactgacat

SEQ ID NO: 190 - RNA, 17 nt, synthetic construct
  source 1..17: mol_type = other RNA
cctatgaaactgacatg

SEQ ID NO: 191 - RNA, 17 nt, synthetic construct
  source 1..17: mol_type = other RNA
ttggtatcttacaggaa

SEQ ID NO: 192 - RNA, 17 nt, synthetic construct
  source 1..17: mol_type = other RNA
acatggagcttttgtat

SEQ ID NO: 193 - RNA, 17 nt, synthetic construct
  source 1..17: mol_type = other RNA
catagggaaattttcac

SEQ ID NO: 194 - RNA, 17 nt, synthetic construct
  source 1..17: mol_type = other RNA
tcacatggagcttttgt

SEQ ID NO: 195 - RNA, 17 nt, synthetic construct
  source 1..17: mol_type = other RNA
gcctttttggtatctta

SEQ ID NO: 196 - RNA, 17 nt, synthetic construct
  source 1..17: mol_type = other RNA
cctttttggtatcttac

SEQ ID NO: 197 - RNA, 17 nt, synthetic construct
  source 1..17: mol_type = other RNA
ctttctttgccagtaca

SEQ ID NO: 198 - RNA, 17 nt, synthetic construct
  source 1..17: mol_type = other RNA
atagggaaattttcaca

SEQ ID NO: 199 - RNA, 17 nt, synthetic construct
  source 1..17: mol_type = other RNA
tactggcaaagaaagaa

SEQ ID NO: 200 - RNA, 17 nt, synthetic construct
  source 1..17: mol_type = other RNA
tttctttgccagtacaa

SEQ ID NO: 201 - RNA, 17 nt, synthetic construct
  source 1..17: mol_type = other RNA
gagaaaagattaaacag

SEQ ID NO: 202 - RNA, 17 nt, synthetic construct
  source 1..17: mol_type = other RNA
tgcctttttggtatctt

SEQ ID NO: 203 - RNA, 17 nt, synthetic construct
  source 1..17: mol_type = other RNA
gttttgcctttttggta

SEQ ID NO: 204 - RNA, 17 nt, synthetic construct
  source 1..17: mol_type = other RNA
ctcaaataaaaagacat

SEQ ID NO: 205 - RNA, 17 nt, synthetic construct
  source 1..17: mol_type = other RNA
agaaaagattaaacagt

SEQ ID NO: 206 - RNA, 17 nt, synthetic construct
  source 1..17: mol_type = other RNA
aatcttttctcaaataa

SEQ ID NO: 207 - RNA, 17 nt, synthetic construct
  source 1..17: mol_type = other RNA
tatttgagaaaagatta

SEQ ID NO: 208 - RNA, 17 nt, synthetic construct
  source 1..17: mol_type = other RNA
gtatttctttctttgcc

SEQ ID NO: 209 - RNA, 17 nt, synthetic construct
  source 1..17: mol_type = other RNA
atttgagaaaagattaa

SEQ ID NO: 210 - RNA, 17 nt, synthetic construct
  source 1..17: mol_type = other RNA
tcaaataaaaagacatg

SEQ ID NO: 211 - RNA, 17 nt, synthetic construct
  source 1..17: mol_type = other RNA
atcttttctcaaataaa

SEQ ID NO: 212 - RNA, 17 nt, synthetic construct
  source 1..17: mol_type = other RNA
ttatttgagaaaagatt

SEQ ID NO: 213 - RNA, 17 nt, synthetic construct
  source 1..17: mol_type = other RNA
ttttgcctttttggtat

SEQ ID NO: 214 - RNA, 17 nt, synthetic construct
  source 1..17: mol_type = other RNA
tatttctttctttgcca

SEQ ID NO: 215 - RNA, 17 nt, synthetic construct
  source 1..17: mol_type = other RNA
ttgttttgcctttttgg

SEQ ID NO: 216 - RNA, 17 nt, synthetic construct
  source 1..17: mol_type = other RNA
tctcaaataaaaagaca

SEQ ID NO: 217 - RNA, 17 nt, synthetic construct
  source 1..17: mol_type = other RNA
tgttttgcctttttggt

SEQ ID NO: 218 - RNA, 17 nt, synthetic construct
  source 1..17: mol_type = other RNA
tgtatttctttctttgc

SEQ ID NO: 219 - RNA, 17 nt, synthetic construct
  source 1..17: mol_type = other RNA
atttttgttttgccttt

SEQ ID NO: 220 - RNA, 17 nt, synthetic construct
  source 1..17: mol_type = other RNA
ctattagatctgtcgcc

SEQ ID NO: 221 - RNA, 17 nt, synthetic construct
  source 1..17: mol_type = other RNA
gatctgtcgccctacct

SEQ ID NO: 222 - RNA, 17 nt, synthetic construct
  source 1..17: mol_type = other RNA
accctgcttataatctc

SEQ ID NO: 223 - RNA, 17 nt, synthetic construct
  source 1..17: mol_type = other RNA
aagaacctatcaaccat

SEQ ID NO: 224 - RNA, 17 nt, synthetic construct
  source 1..17: mol_type = other RNA
gtgcctttcaccctgct

SEQ ID NO: 225 - RNA, 17 nt, synthetic construct
  source 1..17: mol_type = other RNA
taagcagggtgaaaggc

SEQ ID NO: 226 - RNA, 17 nt, synthetic construct
  source 1..17: mol_type = other RNA
caccctgcttataatct

SEQ ID NO: 227 - RNA, 17 nt, synthetic construct
  source 1..17: mol_type = other RNA
catgagagattataagc

SEQ ID NO: 228 - RNA, 17 nt, synthetic construct
  source 1..17: mol_type = other RNA
tagcagactttttaagc

SEQ ID NO: 229 - RNA, 17 nt, synthetic construct
  source 1..17: mol_type = other RNA
attaatggttgataggt

SEQ ID NO: 230 - RNA, 17 nt, synthetic construct
  source 1..17: mol_type = other RNA
atgttagtgcctttcac

SEQ ID NO: 231 - RNA, 17 nt, synthetic construct
  source 1..17: mol_type = other RNA
actgctgttgattaatg

SEQ ID NO: 232 - RNA, 17 nt, synthetic construct
  source 1..17: mol_type = other RNA
ctgctgttgattaatgg

SEQ ID NO: 233 - RNA, 17 nt, synthetic construct
  source 1..17: mol_type = other RNA
tgtctgacagctgtttg

SEQ ID NO: 234 - RNA, 17 nt, synthetic construct
  source 1..17: mol_type = other RNA
agcagactttttaagct

SEQ ID NO: 235 - RNA, 17 nt, synthetic construct
  source 1..17: mol_type = other RNA
atcaacagcagtaaaga

SEQ ID NO: 236 - RNA, 17 nt, synthetic construct
  source 1..17: mol_type = other RNA
cattcctattagatctg

SEQ ID NO: 237 - RNA, 17 nt, synthetic construct
  source 1..17: mol_type = other RNA
ttctgtctgacagctgt

SEQ ID NO: 238 - RNA, 17 nt, synthetic construct
  source 1..17: mol_type = other RNA
atgagagattataagca

SEQ ID NO: 239 - RNA, 17 nt, synthetic construct
  source 1..17: mol_type = other RNA
tctgtctgacagctgtt

SEQ ID NO: 240 - RNA, 17 nt, synthetic construct
  source 1..17: mol_type = other RNA
gcagactttttaagctt

SEQ ID NO: 241 - RNA, 17 nt, synthetic construct
  source 1..17: mol_type = other RNA
atggttgataggttctt

SEQ ID NO: 242 - RNA, 17 nt, synthetic construct
  source 1..17: mol_type = other RNA
ctgtctgacagctgttt

SEQ ID NO: 243 - RNA, 17 nt, synthetic construct
  source 1..17: mol_type = other RNA
aatgttagtgcctttca

SEQ ID NO: 244 - RNA, 17 nt, synthetic construct
  source 1..17: mol_type = other RNA
attcctattagatctgt

SEQ ID NO: 245 - RNA, 17 nt, synthetic construct
  source 1..17: mol_type = other RNA
tttctgtctgacagctg

SEQ ID NO: 246 - RNA, 17 nt, synthetic construct
  source 1..17: mol_type = other RNA
gaagaatatttcatgag

SEQ ID NO: 247 - RNA, 17 nt, synthetic construct
  source 1..17: mol_type = other RNA
tttaatgttagtgcctt

SEQ ID NO: 248 - RNA, 17 nt, synthetic construct
  source 1..17: mol_type = other RNA
agaagaatatttcatga

SEQ ID NO: 249 - RNA, 17 nt, synthetic construct
  source 1..17: mol_type = other RNA
tcattcctattagatct

SEQ ID NO: 250 - RNA, 17 nt, synthetic construct
  source 1..17: mol_type = other RNA
tttactgctgttgatta

SEQ ID NO: 251 - RNA, 17 nt, synthetic construct
  source 1..17: mol_type = other RNA
taagctttctttagaag

SEQ ID NO: 252 - RNA, 17 nt, synthetic construct
  source 1..17: mol_type = other RNA
agctttctttagaagaa

SEQ ID NO: 253 - RNA, 17 nt, synthetic construct
  source 1..17: mol_type = other RNA
ctttactgctgttgatt

SEQ ID NO: 254 - RNA, 17 nt, synthetic construct
  source 1..17: mol_type = other RNA
ttctaaagaaagcttaa

SEQ ID NO: 255 - RNA, 17 nt, synthetic construct
  source 1..17: mol_type = other RNA
ataggttctttaatgtt

SEQ ID NO: 256 - RNA, 17 nt, synthetic construct
  source 1..17: mol_type = other RNA
aaaagtctgctaaaatg

SEQ ID NO: 257 - RNA, 17 nt, synthetic construct
  source 1..17: mol_type = other RNA
aagctttctttagaaga

SEQ ID NO: 258 - RNA, 17 nt, synthetic construct
  source 1..17: mol_type = other RNA
ttaagctttctttagaa

SEQ ID NO: 259 - RNA, 17 nt, synthetic construct
  source 1..17: mol_type = other RNA
taatctctcatgaaata

SEQ ID NO: 260 - RNA, 17 nt, synthetic construct
  source 1..17: mol_type = other RNA
ctttagaagaatatttc

SEQ ID NO: 261 - RNA, 17 nt, synthetic construct
  source 1..17: mol_type = other RNA
tttagaagaatatttca

SEQ ID NO: 262 - RNA, 17 nt, synthetic construct
  source 1..17: mol_type = other RNA
atcgaataagtaatgtg

SEQ ID NO: 263 - RNA, 17 nt, synthetic construct
  source 1..17: mol_type = other RNA
cttattcgattaacact

SEQ ID NO: 264 - RNA, 17 nt, synthetic construct
  source 1..17: mol_type = other RNA
ttcgattaacactttga

SEQ ID NO: 265 - RNA, 17 nt, synthetic construct
  source 1..17: mol_type = other RNA
gaagatctgagctctga

SEQ ID NO: 266 - RNA, 17 nt, synthetic construct
  source 1..17: mol_type = other RNA
tacccactaaagttaat

SEQ ID NO: 267 - RNA, 17 nt, synthetic construct
  source 1..17: mol_type = other RNA
accaaatggattaagat

SEQ ID NO: 268 - RNA, 17 nt, synthetic construct
  source 1..17: mol_type = other RNA
aaagtgttaatcgaata

SEQ ID NO: 269 - RNA, 17 nt, synthetic construct
  source 1..17: mol_type = other RNA
aaagaaattctacccac

SEQ ID NO: 270 - RNA, 17 nt, synthetic construct
  source 1..17: mol_type = other RNA
agtgggtagaatttctt

SEQ ID NO: 271 - RNA, 17 nt, synthetic construct
  source 1..17: mol_type = other RNA
gattaacactttgaaga

SEQ ID NO: 272 - RNA, 17 nt, synthetic construct
  source 1..17: mol_type = other RNA
atgaacatcttaatcca

SEQ ID NO: 273 - RNA, 17 nt, synthetic construct
  source 1..17: mol_type = other RNA
ggtgaatatattattgg

SEQ ID NO: 274 - RNA, 17 nt, synthetic construct
  source 1..17: mol_type = other RNA
aacagaaaagcatacac

SEQ ID NO: 275 - RNA, 17 nt, synthetic construct
  source 1..17: mol_type = other RNA
tctgttaaagaggaagt

SEQ ID NO: 276 - RNA, 17 nt, synthetic construct
  source 1..17: mol_type = other RNA
tgttaaagaggaagtta

SEQ ID NO: 277 - RNA, 17 nt, synthetic construct
  source 1..17: mol_type = other RNA
tcttcaaagtgttaatc

SEQ ID NO: 278 - RNA, 17 nt, synthetic construct
  source 1..17: mol_type = other RNA
ctgttaaagaggaagtt

SEQ ID NO: 279 - RNA, 17 nt, synthetic construct
  source 1..17: mol_type = other RNA
gtgaatatattattgga

SEQ ID NO: 280 - RNA, 17 nt, synthetic construct
  source 1..17: mol_type = other RNA
atccatttggtgaatat

SEQ ID NO: 281 - RNA, 17 nt, synthetic construct
  source 1..17: mol_type = other RNA
gtgggtagaatttcttt

SEQ ID NO: 282 - RNA, 17 nt, synthetic construct
  source 1..17: mol_type = other RNA
aagaggaagttagaaga

SEQ ID NO: 283 - RNA, 17 nt, synthetic construct
  source 1..17: mol_type = other RNA
taacttcctctttaaca

SEQ ID NO: 284 - RNA, 17 nt, synthetic construct
  source 1..17: mol_type = other RNA
aagataattcatgaaca

SEQ ID NO: 285 - RNA, 17 nt, synthetic construct
  source 1..17: mol_type = other RNA
agatgttcatgaattat

SEQ ID NO: 286 - RNA, 17 nt, synthetic construct
  source 1..17: mol_type = other RNA
taaattaactttagtgg

SEQ ID NO: 287 - RNA, 17 nt, synthetic construct
  source 1..17: mol_type = other RNA
aaaagaaattctaccca

SEQ ID NO: 288 - RNA, 17 nt, synthetic construct
  source 1..17: mol_type = other RNA
ctctttaacagaaaagc

SEQ ID NO: 289 - RNA, 17 nt, synthetic construct
  source 1..17: mol_type = other RNA
gaagtaaaatataatag

SEQ ID NO: 290 - RNA, 17 nt, synthetic construct
  source 1..17: mol_type = other RNA
acagaaaagcatacaca

SEQ ID NO: 291 - RNA, 17 nt, synthetic construct
  source 1..17: mol_type = other RNA
atgaattatcttcaaag

SEQ ID NO: 292 - RNA, 17 nt, synthetic construct
  source 1..17: mol_type = other RNA
acactttgaagataatt

SEQ ID NO: 293 - RNA, 17 nt, synthetic construct
  source 1..17: mol_type = other RNA
tacttctaaattaactt

SEQ ID NO: 294 - RNA, 17 nt, synthetic construct
  source 1..17: mol_type = other RNA
cttctaaattaacttta

SEQ ID NO: 295 - RNA, 17 nt, synthetic construct
  source 1..17: mol_type = other RNA
acttctaaattaacttt

SEQ ID NO: 296 - RNA, 17 nt, synthetic construct
  source 1..17: mol_type = other RNA
agaagtaaaatataata

SEQ ID NO: 297 - RNA, 17 nt, synthetic construct
  source 1..17: mol_type = other RNA
atttagaagtaaaatat

SEQ ID NO: 298 - RNA, 17 nt, synthetic construct
  source 1..17: mol_type = other RNA
ctttaaaagaaattcta

SEQ ID NO: 299 - RNA, 17 nt, synthetic construct
  source 1..17: mol_type = other RNA
ttggatttctattatat

SEQ ID NO: 300 - RNA, 17 nt, synthetic construct
  source 1..17: mol_type = other RNA
ctattatattttacttc

SEQ ID NO: 301 - RNA, 17 nt, synthetic construct
  source 1..17: mol_type = other RNA
gatttctattatatttt

SEQ ID NO: 302 - RNA, 17 nt, synthetic construct
  source 1..17: mol_type = other RNA
tattttacttctaaatt

SEQ ID NO: 303 - RNA, 17 nt, synthetic construct
  source 1..17: mol_type = other RNA
tattatattttacttct

SEQ ID NO: 304 - RNA, 17 nt, synthetic construct
  source 1..17: mol_type = other RNA
caggctccaatagtggt

SEQ ID NO: 305 - RNA, 17 nt, synthetic construct
  source 1..17: mol_type = other RNA
gagcctgtaagtatact

SEQ ID NO: 306 - RNA, 17 nt, synthetic construct
  source 1..17: mol_type = other RNA
aacaaatagctagagcc

SEQ ID NO: 307 - RNA, 17 nt, synthetic construct
  source 1..17: mol_type = other RNA
gtgttattgactgtgca

SEQ ID NO: 308 - RNA, 17 nt, synthetic construct
  source 1..17: mol_type = other RNA
gaacaaatagctagagc

SEQ ID NO: 309 - RNA, 17 nt, synthetic construct
  source 1..17: mol_type = other RNA
ggctctagctatttgtt

SEQ ID NO: 310 - RNA, 17 nt, synthetic construct
  source 1..17: mol_type = other RNA
gcacagtcaataacaca

SEQ ID NO: 311 - RNA, 17 nt, synthetic construct
  source 1..17: mol_type = other RNA
tgcacagtcaataacac

SEQ ID NO: 312 - RNA, 17 nt, synthetic construct
  source 1..17: mol_type = other RNA
gttcaaaagtgcaacta

SEQ ID NO: 313 - RNA, 17 nt, synthetic construct
  source 1..17: mol_type = other RNA
gctctagctatttgttc

SEQ ID NO: 314 - RNA, 17 nt, synthetic construct
  source 1..17: mol_type = other RNA
ttgcacagtcaataaca

SEQ ID NO: 315 - RNA, 17 nt, synthetic construct
  source 1..17: mol_type = other RNA
tgaacaaatagctagag

SEQ ID NO: 316 - RNA, 17 nt, synthetic construct
  source 1..17: mol_type = other RNA
tgttattgactgtgcaa

SEQ ID NO: 317 - RNA, 17 nt, synthetic construct
  source 1..17: mol_type = other RNA
cacagtcaataacacaa

SEQ ID NO: 318 - RNA, 17 nt, synthetic construct
  source 1..17: mol_type = other RNA
tctctcacccagtcatc

SEQ ID NO: 319 - RNA, 17 nt, synthetic construct
  source 1..17: mol_type = other RNA
ttgactgtgcaaaaagt

SEQ ID NO: 320 - RNA, 17 nt, synthetic construct
  source 1..17: mol_type = other RNA
tctttggctctagctat

SEQ ID NO: 321 - RNA, 17 nt, synthetic construct
  source 1..17: mol_type = other RNA
tctctctcacccagtca

SEQ ID NO: 322 - RNA, 17 nt, synthetic construct
  source 1..17: mol_type = other RNA
actgtgcaaaaagtctt

SEQ ID NO: 323 - RNA, 17 nt, synthetic construct
  source 1..17: mol_type = other RNA
ttcaaaagtgcaactat

SEQ ID NO: 324 - RNA, 17 nt, synthetic construct
  source 1..17: mol_type = other RNA
atagttgcacttttgaa

SEQ ID NO: 325 - RNA, 17 nt, synthetic construct
  source 1..17: mol_type = other RNA
caattctaaagatagag

SEQ ID NO: 326 - RNA, 17 nt, synthetic construct
  source 1..17: mol_type = other RNA
gtttcaattctaaagat

SEQ ID NO: 327 - RNA, 17 nt, synthetic construct
  source 1..17: mol_type = other RNA
cacttttgaacaaatag

SEQ ID NO: 328 - RNA, 17 nt, synthetic construct
  source 1..17: mol_type = other RNA
aaaagtgcaactatgaa

SEQ ID NO: 329 - RNA, 17 nt, synthetic construct
  source 1..17: mol_type = other RNA
ctctctcacccagtcat

SEQ ID NO: 330 - RNA, 17 nt, synthetic construct
  source 1..17: mol_type = other RNA
agaattgaaacaaattt

SEQ ID NO: 331 - RNA, 17 nt, synthetic construct
  source 1..17: mol_type = other RNA
gaattgaaacaaatttt

SEQ ID NO: 332 - RNA, 17 nt, synthetic construct
  source 1..17: mol_type = other RNA
tctctatctttagaatt

SEQ ID NO: 333 - RNA, 17 nt, synthetic construct
  source 1..17: mol_type = other RNA
aattctaaagatagaga

SEQ ID NO: 334 - RNA, 17 nt, synthetic construct
  source 1..17: mol_type = other RNA
taaagatagagataaac

SEQ ID NO: 335 - RNA, 17 nt, synthetic construct
  source 1..17: mol_type = other RNA
aaacaaattttctctct

SEQ ID NO: 336 - RNA, 17 nt, synthetic construct
  source 1..17: mol_type = other RNA
tttcaattctaaagata

SEQ ID NO: 337 - RNA, 17 nt, synthetic construct
  source 1..17: mol_type = other RNA
gctcctactcagactgt

SEQ ID NO: 338 - RNA, 17 nt, synthetic construct
  source 1..17: mol_type = other RNA
agctcctactcagactg

SEQ ID NO: 339 - RNA, 17 nt, synthetic construct
  source 1..17: mol_type = other RNA
ggctcaaattgttactc

SEQ ID NO: 340 - RNA, 17 nt, synthetic construct
  source 1..17: mol_type = other RNA
tagctcctactcagact

SEQ ID NO: 341 - RNA, 17 nt, synthetic construct
  source 1..17: mol_type = other RNA
gagccaaactcttattc

SEQ ID NO: 342 - RNA, 17 nt, synthetic construct
  source 1..17: mol_type = other RNA
gggtttttgcaaaaagg

SEQ ID NO: 343 - RNA, 17 nt, synthetic construct
  source 1..17: mol_type = other RNA
gctcaaattgttactct

SEQ ID NO: 344 - RNA, 17 nt, synthetic construct
  source 1..17: mol_type = other RNA
aaattggctctttagct

SEQ ID NO: 345 - RNA, 17 nt, synthetic construct
  source 1..17: mol_type = other RNA
gctctttagcttgtgtt

SEQ ID NO: 346 - RNA, 17 nt, synthetic construct
  source 1..17: mol_type = other RNA
agccaaactcttattca

SEQ ID NO: 347 - RNA, 17 nt, synthetic construct
  source 1..17: mol_type = other RNA
cctttttgcaaaaaccc

SEQ ID NO: 348 - RNA, 17 nt, synthetic construct
  source 1..17: mol_type = other RNA
aagagtaacaatttgag

SEQ ID NO: 349 - RNA, 17 nt, synthetic construct
  source 1..17: mol_type = other RNA
gaaacacaagctaaaga

SEQ ID NO: 350 - RNA, 17 nt, synthetic construct
  source 1..17: mol_type = other RNA
gacttattgttattgaa

SEQ ID NO: 351 - RNA, 17 nt, synthetic construct
  source 1..17: mol_type = other RNA
gcaaaaacccaaaatat

SEQ ID NO: 352 - RNA, 17 nt, synthetic construct
  source 1..17: mol_type = other RNA
ggtttttgcaaaaagga

SEQ ID NO: 353 - RNA, 17 nt, synthetic construct
  source 1..17: mol_type = other RNA
tcctttttgcaaaaacc

SEQ ID NO: 354 - RNA, 17 nt, synthetic construct
  source 1..17: mol_type = other RNA
caataacaataagtcaa

SEQ ID NO: 355 - RNA, 17 nt, synthetic construct
  source 1..17: mol_type = other RNA
attgaagagtaacaatt

SEQ ID NO: 356 - RNA, 17 nt, synthetic construct
  source 1..17: mol_type = other RNA
ttgaaattggctcttta

SEQ ID NO: 357 - RNA, 17 nt, synthetic construct
  source 1..17: mol_type = other RNA
tgcaaaaacccaaaata

SEQ ID NO: 358 - RNA, 17 nt, synthetic construct
  source 1..17: mol_type = other RNA
ttattgaaattggctct

SEQ ID NO: 359 - RNA, 17 nt, synthetic construct
  source 1..17: mol_type = other RNA
agcttgtgtttctaatt

SEQ ID NO: 360 - RNA, 17 nt, synthetic construct
  source 1..17: mol_type = other RNA
ttgcaaaaacccaaaat

SEQ ID NO: 361 - RNA, 17 nt, synthetic construct
  source 1..17: mol_type = other RNA
ttgttattgaaattggc

SEQ ID NO: 362 - RNA, 17 nt, synthetic construct
  source 1..17: mol_type = other RNA
gcttgtgtttctaattt

SEQ ID NO: 363 - RNA, 17 nt, synthetic construct
  source 1..17: mol_type = other RNA
ttcatgacattatatat

SEQ ID NO: 364 - RNA, 17 nt, synthetic construct
  source 1..17: mol_type = other RNA
ctcttcaattaaatttg

SEQ ID NO: 365 - RNA, 17 nt, synthetic construct
  source 1..17: mol_type = other RNA
ttactcttcaattaaat

SEQ ID NO: 366 - RNA, 17 nt, synthetic construct
  source 1..17: mol_type = other RNA
caaaaacccaaaatatt

SEQ ID NO: 367 - RNA, 17 nt, synthetic construct
  source 1..17: mol_type = other RNA
ttcctttttgcaaaaac

SEQ ID NO: 368 - RNA, 17 nt, synthetic construct
  source 1..17: mol_type = other RNA
ctttttgcaaaaaccca

SEQ ID NO: 369 - RNA, 17 nt, synthetic construct
  source 1..17: mol_type = other RNA
acttattgttattgaaa

SEQ ID NO: 370 - RNA, 17 nt, synthetic construct
  source 1..17: mol_type = other RNA
aatttgacttattgtta

SEQ ID NO: 371 - RNA, 17 nt, synthetic construct
  source 1..17: mol_type = other RNA
aattaaatttgacttat

SEQ ID NO: 372 - RNA, 17 nt, synthetic construct
  source 1..17: mol_type = other RNA
tttcctttttgcaaaaa

SEQ ID NO: 373 - RNA, 17 nt, synthetic construct
  source 1..17: mol_type = other RNA
gcaaaaaggaaaaaaga

SEQ ID NO: 374 - RNA, 17 nt, synthetic construct
  source 1..17: mol_type = other RNA
tctttttcttctttttt

SEQ ID NO: 375 - RNA, 17 nt, synthetic construct
  source 1..17: mol_type = other RNA
caaaaaggaaaaaagaa

SEQ ID NO: 376 - RNA, 17 nt, synthetic construct
  source 1..17: mol_type = other RNA
ttttttcctttttgcaa

SEQ ID NO: 377 - RNA, 17 nt, synthetic construct
  source 1..17: mol_type = other RNA
ttcttttttcctttttg

SEQ ID NO: 378 - RNA, 17 nt, synthetic construct
  source 1..17: mol_type = other RNA
tgtttctaatttttctt

SEQ ID NO: 379 - RNA, 17 nt, synthetic construct
  source 1..17: mol_type = other RNA
ctaatttttctttttct

SEQ ID NO: 380 - RNA, 17 nt, synthetic construct
  source 1..17: mol_type = other RNA
tttttcttcttttttcc

SEQ ID NO: 381 - RNA, 17 nt, synthetic construct
  source 1..17: mol_type = other RNA
tcttcttttttcctttt

SEQ ID NO: 382 - RNA, 17 nt, synthetic construct
  source 1..17: mol_type = other RNA
taatttttctttttctt

SEQ ID NO: 383 - RNA, 17 nt, synthetic construct
  source 1..17: mol_type = other RNA
ttcttcttttttccttt

SEQ ID NO: 384 - RNA, 17 nt, synthetic construct
  source 1..17: mol_type = other RNA
cttcttttttccttttt

SEQ ID NO: 385 - RNA, 17 nt, synthetic construct
  source 1..17: mol_type = other RNA
ctttttcttcttttttc

SEQ ID NO: 386 - RNA, 17 nt, synthetic construct
  source 1..17: mol_type = other RNA
ttctttttcttcttttt

SEQ ID NO: 387 - RNA, 17 nt, synthetic construct
  source 1..17: mol_type = other RNA
cccaccagttcttaggc

SEQ ID NO: 388 - RNA, 17 nt, synthetic construct
  source 1..17: mol_type = other RNA
cctaagaactggtggga

SEQ ID NO: 389 - RNA, 17 nt, synthetic construct
  source 1..17: mol_type = other RNA
ccaccagttcttaggca

SEQ ID NO: 390 - RNA, 17 nt, synthetic construct
  source 1..17: mol_type = other RNA
atgatcatctcgttgat

SEQ ID NO: 391 - RNA, 17 nt, synthetic construct
  source 1..17: mol_type = other RNA
tgcttgatgatcatctc

SEQ ID NO: 392 - RNA, 17 nt, synthetic construct
  source 1..17: mol_type = other RNA
caccaatcactttactc

SEQ ID NO: 393 - RNA, 17 nt, synthetic construct
  source 1..17: mol_type = other RNA
tccaccaatcactttac

SEQ ID NO: 394 - RNA, 17 nt, synthetic construct
  source 1..17: mol_type = other RNA
actctcctagaccattt

SEQ ID NO: 395 - RNA, 17 nt, synthetic construct
  source 1..17: mol_type = other RNA
ccaccaatcactttact

SEQ ID NO: 396 - RNA, 17 nt, synthetic construct
  source 1..17: mol_type = other RNA
ctctcctagaccatttc

SEQ ID NO: 397 - RNA, 17 nt, synthetic construct
  source 1..17: mol_type = other RNA
gctgagagagaaacagt

SEQ ID NO: 398 - RNA, 17 nt, synthetic construct
  source 1..17: mol_type = other RNA
ctctcactattcagcct

SEQ ID NO: 399 - RNA, 17 nt, synthetic construct
  source 1..17: mol_type = other RNA
tcataccttctgcttga

SEQ ID NO: 400 - RNA, 17 nt, synthetic construct
  source 1..17: mol_type = other RNA
ttaggcaactgtttctc

SEQ ID NO: 401 - RNA, 17 nt, synthetic construct
  source 1..17: mol_type = other RNA
ctcataccttctgcttg

SEQ ID NO: 402 - RNA, 17 nt, synthetic construct
  source 1..17: mol_type = other RNA
ggcaactgtttctctct

SEQ ID NO: 403 - RNA, 17 nt, synthetic construct
  source 1..17: mol_type = other RNA
ctctctcagcaaacaca

SEQ ID NO: 404 - RNA, 17 nt, synthetic construct
  source 1..17: mol_type = other RNA
aaagaaaaacttctgcc

SEQ ID NO: 405 - RNA, 17 nt, synthetic construct
  source 1..17: mol_type = other RNA
tctctcagcaaacacat

SEQ ID NO: 406 - RNA, 17 nt, synthetic construct
  source 1..17: mol_type = other RNA
aagaaaaacttctgcca

SEQ ID NO: 407 - RNA, 17 nt, synthetic construct
  source 1..17: mol_type = other RNA
ttctcataccttctgct

SEQ ID NO: 408 - RNA, 17 nt, synthetic construct
  source 1..17: mol_type = other RNA
ctgagagagaaacagtt

SEQ ID NO: 409 - RNA, 17 nt, synthetic construct
  source 1..17: mol_type = other RNA
gcagaagtttttcttta

SEQ ID NO: 410 - RNA, 17 nt, synthetic construct
  source 1..17: mol_type = other RNA
taaagaaaaacttctgc

SEQ ID NO: 411 - RNA, 17 nt, synthetic construct
  source 1..17: mol_type = other RNA
gtggaaaatcttcattt

SEQ ID NO: 412 - RNA, 17 nt, synthetic construct
  source 1..17: mol_type = other RNA
tttctcataccttctgc

SEQ ID NO: 413 - RNA, 17 nt, synthetic construct
  source 1..17: mol_type = other RNA
aaatgaagattttccac

SEQ ID NO: 414 - RNA, 17 nt, synthetic construct
  source 1..17: mol_type = other RNA
tgccaacttttatcatt

SEQ ID NO: 415 - RNA, 17 nt, synthetic construct
  source 1..17: mol_type = other RNA
tctcataccttctgctt

SEQ ID NO: 416 - RNA, 17 nt, synthetic construct
  source 1..17: mol_type = other RNA
aaaatgaagattttcca

SEQ ID NO: 417 - RNA, 17 nt, synthetic construct
  source 1..17: mol_type = other RNA
tcattttttctcatacc

SEQ ID NO: 418 - RNA, 17 nt, synthetic construct
  source 1..17: mol_type = other RNA
tatcattttttctcata

SEQ ID NO: 419 - RNA, 17 nt, synthetic construct
  source 1..17: mol_type = other RNA
atcattttttctcatac

SEQ ID NO: 420 - RNA, 17 nt, synthetic construct
  source 1..17: mol_type = other RNA
ctttaaaatgaagattt

SEQ ID NO: 421 - RNA, 17 nt, synthetic construct
  source 1..17: mol_type = other RNA
tctttaaaatgaagatt

SEQ ID NO: 422 - RNA, 17 nt, synthetic construct
  source 1..17: mol_type = other RNA
ttctttaaaatgaagat

SEQ ID NO: 423 - RNA, 17 nt, synthetic construct
  source 1..17: mol_type = other RNA
attttaaagaaaaactt

SEQ ID NO: 424 - RNA, 17 nt, synthetic construct
  source 1..17: mol_type = other RNA
tttaaaatgaagatttt

SEQ ID NO: 425 - RNA, 17 nt, synthetic construct
  source 1..17: mol_type = other RNA
atcatcctagccataac

SEQ ID NO: 426 - RNA, 17 nt, synthetic construct
  source 1..17: mol_type = other RNA
tgttatggctaggatga

SEQ ID NO: 427 - RNA, 17 nt, synthetic construct
  source 1..17: mol_type = other RNA
aatgtaacttccaaacg

SEQ ID NO: 428 - RNA, 17 nt, synthetic construct
  source 1..17: mol_type = other RNA
attgtgttatggctagg

SEQ ID NO: 429 - RNA, 17 nt, synthetic construct
  source 1..17: mol_type = other RNA
ttcatcatcctagccat

SEQ ID NO: 430 - RNA, 17 nt, synthetic construct
  source 1..17: mol_type = other RNA
atcccactgattctgaa

SEQ ID NO: 431 - RNA, 17 nt, synthetic construct
  source 1..17: mol_type = other RNA
aaatgtctcctccagac

SEQ ID NO: 432 - RNA, 17 nt, synthetic construct
  source 1..17: mol_type = other RNA
ttcattgtgttatggct

SEQ ID NO: 433 - RNA, 17 nt, synthetic construct
  source 1..17: mol_type = other RNA
agaatcagtgggatgaa

SEQ ID NO: 434 - RNA, 17 nt, synthetic construct
  source 1..17: mol_type = other RNA
aaaatgtctcctccaga

SEQ ID NO: 435 - RNA, 17 nt, synthetic construct
  source 1..17: mol_type = other RNA
caaacgttatctcacat

SEQ ID NO: 436 - RNA, 17 nt, synthetic construct
  source 1..17: mol_type = other RNA
ggaagttacatttaaaa

SEQ ID NO: 437 - RNA, 17 nt, synthetic construct
  source 1..17: mol_type = other RNA
tgaattctttcaactag

SEQ ID NO: 438 - RNA, 17 nt, synthetic construct
  source 1..17: mol_type = other RNA
caactagaataaaagga

SEQ ID NO: 439 - RNA, 17 nt, synthetic construct
  source 1..17: mol_type = other RNA
tctcacatttatgttgc

SEQ ID NO: 440 - RNA, 17 nt, synthetic construct
  source 1..17: mol_type = other RNA
attctagttgaaagaat

SEQ ID NO: 441 - RNA, 17 nt, synthetic construct
  source 1..17: mol_type = other RNA
catttaaaatgtctcct

SEQ ID NO: 442 - RNA, 17 nt, synthetic construct
  source 1..17: mol_type = other RNA
tagttgaaagaattcag

SEQ ID NO: 443 - RNA, 17 nt, synthetic construct
  source 1..17: mol_type = other RNA
aaagaattcagaatcag

SEQ ID NO: 444 - RNA, 17 nt, synthetic construct
  source 1..17: mol_type = other RNA
aataagcaacataaatg

SEQ ID NO: 445 - RNA, 17 nt, synthetic construct
  source 1..17: mol_type = other RNA
aactagaataaaaggaa

SEQ ID NO: 446 - RNA, 17 nt, synthetic construct
  source 1..17: mol_type = other RNA
ccttttattctagttga

SEQ ID NO: 447 - RNA, 17 nt, synthetic construct
  source 1..17: mol_type = other RNA
ttctagttgaaagaatt

SEQ ID NO: 448 - RNA, 17 nt, synthetic construct
  source 1..17: mol_type = other RNA
tattctagttgaaagaa

SEQ ID NO: 449 - RNA, 17 nt, synthetic construct
  source 1..17: mol_type = other RNA
ttaaataagcaacataa

SEQ ID NO: 450 - RNA, 17 nt, synthetic construct
  source 1..17: mol_type = other RNA
taaataagcaacataaa

SEQ ID NO: 451 - RNA, 17 nt, synthetic construct
  source 1..17: mol_type = other RNA
tttaaataagcaacata

SEQ ID NO: 452 - RNA, 17 nt, synthetic construct
  source 1..17: mol_type = other RNA
aaaaattattcattgtg

SEQ ID NO: 453 - RNA, 17 nt, synthetic construct
  source 1..17: mol_type = other RNA
aaataagcaacataaat

SEQ ID NO: 454 - RNA, 17 nt, synthetic construct
  source 1..17: mol_type = other RNA
tccttttattctagttg

SEQ ID NO: 455 - RNA, 17 nt, synthetic construct
  source 1..17: mol_type = other RNA
gaagttacatttaaaat

SEQ ID NO: 456 - RNA, 17 nt, synthetic construct
  source 1..17: mol_type = other RNA
cttttattctagttgaa

SEQ ID NO: 457 - RNA, 17 nt, synthetic construct
  source 1..17: mol_type = other RNA
actactatatatttatt

SEQ ID NO: 458 - RNA, 17 nt, synthetic construct
  source 1..17: mol_type = other RNA
aaaaaattattcattgt

SEQ ID NO: 459 - RNA, 17 nt, synthetic construct
  source 1..17: mol_type = other RNA
ttccttttattctagtt

SEQ ID NO: 460 - RNA, 17 nt, synthetic construct
  source 1..17: mol_type = other RNA
ctactatatatttattt

SEQ ID NO: 461 - RNA, 17 nt, synthetic construct
  source 1..17: mol_type = other RNA
tttttccttttattcta

SEQ ID NO: 462 - RNA, 17 nt, synthetic construct
  source 1..17: mol_type = other RNA
atttttccttttattct

SEQ ID NO: 463 - RNA, 17 nt, synthetic construct
  source 1..17: mol_type = other RNA
cagctttaacgtgattt

SEQ ID NO: 464 - RNA, 17 nt, synthetic construct
  source 1..17: mol_type = other RNA
gatactaaccttggttt

SEQ ID NO: 465 - RNA, 17 nt, synthetic construct
  source 1..17: mol_type = other RNA
atttcagctttaacgtg

SEQ ID NO: 466 - RNA, 17 nt, synthetic construct
  source 1..17: mol_type = other RNA
atactaaccttggtttc

SEQ ID NO: 467 - RNA, 17 nt, synthetic construct
  source 1..17: mol_type = other RNA
gtatcaaagataccttt

SEQ ID NO: 468 - RNA, 17 nt, synthetic construct
  source 1..17: mol_type = other RNA
acagaaaatcacgttaa

SEQ ID NO: 469 - RNA, 17 nt, synthetic construct
  source 1..17: mol_type = other RNA
ttaacagaaaatcacgt

SEQ ID NO: 470 - RNA, 17 nt, synthetic construct
  source 1..17: mol_type = other RNA
tatcaaatgtaaccagt

SEQ ID NO: 471 - RNA, 17 nt, synthetic construct
  source 1..17: mol_type = other RNA
aattacaatctatggta

SEQ ID NO: 472 - RNA, 17 nt, synthetic construct
  source 1..17: mol_type = other RNA
atcaaatgtaaccagta

SEQ ID NO: 473 - RNA, 17 nt, synthetic construct
  source 1..17: mol_type = other RNA
agctttaacgtgatttt

SEQ ID NO: 474 - RNA, 17 nt, synthetic construct
  source 1..17: mol_type = other RNA
acgtgattttctgttaa

SEQ ID NO: 475 - RNA, 17 nt, synthetic construct
  source 1..17: mol_type = other RNA
aacgtgattttctgtta

SEQ ID NO: 476 - RNA, 17 nt, synthetic construct
  source 1..17: mol_type = other RNA
tcaaatgtaaccagtat

SEQ ID NO: 477 - RNA, 17 nt, synthetic construct
  source 1..17: mol_type = other RNA
taccatagattgtaatt

SEQ ID NO: 478 - RNA, 17 nt, synthetic construct
  source 1..17: mol_type = other RNA
caatctatggtataatt

SEQ ID NO: 479 - RNA, 17 nt, synthetic construct
  source 1..17: mol_type = other RNA
aagctgaaatgaacagt

SEQ ID NO: 480 - RNA, 17 nt, synthetic construct
  source 1..17: mol_type = other RNA
gataaaattataccata

SEQ ID NO: 481 - RNA, 17 nt, synthetic construct
  source 1..17: mol_type = other RNA
ctgttaataactttaca

SEQ ID NO: 482 - RNA, 17 nt, synthetic construct
  source 1..17: mol_type = other RNA
tctgttaataactttac

SEQ ID NO: 483 - RNA, 17 nt, synthetic construct
  source 1..17: mol_type = other RNA
aaataaaatactggtta

SEQ ID NO: 484 - RNA, 17 nt, synthetic construct
  source 1..17: mol_type = other RNA
aatgatgtttaatgtaa

SEQ ID NO: 485 - RNA, 17 nt, synthetic construct
  source 1..17: mol_type = other RNA
aaaaggtatctttgata

SEQ ID NO: 486 - RNA, 17 nt, synthetic construct
  source 1..17: mol_type = other RNA
acattaaacatcattaa

SEQ ID NO: 487 - RNA, 17 nt, synthetic construct
  source 1..17: mol_type = other RNA
atgtaaagttattaaca

SEQ ID NO: 488 - RNA, 17 nt, synthetic construct
  source 1..17: mol_type = other RNA
ataaaattataccatag

SEQ ID NO: 489 - RNA, 17 nt, synthetic construct
  source 1..17: mol_type = other RNA
taaaaaggtatctttga

SEQ ID NO: 490 - RNA, 17 nt, synthetic construct
  source 1..17: mol_type = other RNA
aaaaaggtatctttgat

SEQ ID NO: 491 - RNA, 17 nt, synthetic construct
  source 1..17: mol_type = other RNA
aatgtaaagttattaac

SEQ ID NO: 492 - RNA, 17 nt, synthetic construct
  source 1..17: mol_type = other RNA
aacatcattaaattaca

SEQ ID NO: 493 - RNA, 17 nt, synthetic construct
  source 1..17: mol_type = other RNA
atgatgtttaatgtaaa

SEQ ID NO: 494 - RNA, 17 nt, synthetic construct
  source 1..17: mol_type = other RNA
aaaataaaatactggtt

SEQ ID NO: 495 - RNA, 17 nt, synthetic construct
  source 1..17: mol_type = other RNA
ttaaaataaaatactgg

SEQ ID NO: 496 - RNA, 17 nt, synthetic construct
  source 1..17: mol_type = other RNA
taaaataaaatactggt

SEQ ID NO: 497 - RNA, 17 nt, synthetic construct
  source 1..17: mol_type = other RNA
ctgtgattttcttttgg

SEQ ID NO: 498 - RNA, 17 nt, synthetic construct
  source 1..17: mol_type = other RNA
tgttaataactttacat

SEQ ID NO: 499 - RNA, 17 nt, synthetic construct
  source 1..17: mol_type = other RNA
tattttaaaaaggtatc

SEQ ID NO: 500 - RNA, 17 nt, synthetic construct
  source 1..17: mol_type = other RNA
taatttaatgatgttta

SEQ ID NO: 501 - RNA, 17 nt, synthetic construct
  source 1..17: mol_type = other RNA
gtttctgtgattttctt

SEQ ID NO: 502 - RNA, 17 nt, synthetic construct
  source 1..17: mol_type = other RNA
attttaaaaaggtatct

SEQ ID NO: 503 - RNA, 17 nt, synthetic construct
  source 1..17: mol_type = other RNA
ttttaaaaaggtatctt

SEQ ID NO: 504 - RNA, 17 nt, synthetic construct
  source 1..17: mol_type = other RNA
catttgataaaattata

SEQ ID NO: 505 - RNA, 17 nt, synthetic construct
  source 1..17: mol_type = other RNA
tgtgattttcttttgga

SEQ ID NO: 506 - RNA, 17 nt, synthetic construct
  source 1..17: mol_type = other RNA
catctactgtataggga

SEQ ID NO: 507 - RNA, 17 nt, synthetic construct
  source 1..17: mol_type = other RNA
gctctggcctgtcctaa

SEQ ID NO: 508 - RNA, 17 nt, synthetic construct
  source 1..17: mol_type = other RNA
cattcaactgttgcctc

SEQ ID NO: 509 - RNA, 17 nt, synthetic construct
  source 1..17: mol_type = other RNA
tgaaggtgttcttgtac

SEQ ID NO: 510 - RNA, 17 nt, synthetic construct
  source 1..17: mol_type = other RNA
cttttggattgcatcta

SEQ ID NO: 511 - RNA, 17 nt, synthetic construct
  source 1..17: mol_type = other RNA
cttagcttccagccatt

SEQ ID NO: 512 - RNA, 17 nt, synthetic construct
  source 1..17: mol_type = other RNA
ttttggattgcatctac

SEQ ID NO: 513 - RNA, 17 nt, synthetic construct
  source 1..17: mol_type = other RNA
ggattgcatctactgta

SEQ ID NO: 514 - RNA, 17 nt, synthetic construct
  source 1..17: mol_type = other RNA
aacatttcattcaactg

SEQ ID NO: 515 - RNA, 17 nt, synthetic construct
  source 1..17: mol_type = other RNA
ttgtacttcatcccact

SEQ ID NO: 516 - RNA, 17 nt, synthetic construct
  source 1..17: mol_type = other RNA
tcttttggattgcatct

SEQ ID NO: 517 - RNA, 17 nt, synthetic construct
  source 1..17: mol_type = other RNA
acatttcattcaactgt

SEQ ID NO: 518 - RNA, 17 nt, synthetic construct
  source 1..17: mol_type = other RNA
ttccttagcttccagcc

SEQ ID NO: 519 - RNA, 17 nt, synthetic construct
  source 1..17: mol_type = other RNA
gattgcatctactgtat

SEQ ID NO: 520 - RNA, 17 nt, synthetic construct
  source 1..17: mol_type = other RNA
catgactcaagcttggc

SEQ ID NO: 521 - RNA, 17 nt, synthetic construct
  source 1..17: mol_type = other RNA
attcaactgttgcctcc

SEQ ID NO: 522 - RNA, 17 nt, synthetic construct
  source 1..17: mol_type = other RNA
tgttgaatcctttaaca

SEQ ID NO: 523 - RNA, 17 nt, synthetic construct
  source 1..17: mol_type = other RNA
tggattgcatctactgt

SEQ ID NO: 524 - RNA, 17 nt, synthetic construct
  source 1..17: mol_type = other RNA
gcttccagccattgtgt

SEQ ID NO: 525 - RNA, 17 nt, synthetic construct
  source 1..17: mol_type = other RNA
cagccattgtgttgaat

SEQ ID NO: 526 - RNA, 17 nt, synthetic construct
  source 1..17: mol_type = other RNA
cctccggttctgaaggt

SEQ ID NO: 527 - RNA, 17 nt, synthetic construct
  source 1..17: mol_type = other RNA
tacttcatcccactgat

SEQ ID NO: 528 - RNA, 17 nt, synthetic construct
  source 1..17: mol_type = other RNA
aactgttgcctccggtt

SEQ ID NO: 529 - RNA, 17 nt, synthetic construct
  source 1..17: mol_type = other RNA
aaggattcaacacaatg

SEQ ID NO: 530 - RNA, 17 nt, synthetic construct
  source 1..17: mol_type = other RNA
aacacaatggctggaag

SEQ ID NO: 531 - RNA, 17 nt, synthetic construct
  source 1..17: mol_type = other RNA
ggacaggccagagccaa

SEQ ID NO: 532 - RNA, 17 nt, synthetic construct
  source 1..17: mol_type = other RNA
agtcatggaaggagggt

SEQ ID NO: 533 - RNA, 17 nt, synthetic construct
  source 1..17: mol_type = other RNA
agaaccggaggcaacag

SEQ ID NO: 534 - RNA, 17 nt, synthetic construct
  source 1..17: mol_type = other RNA
tataacttgatcaagca

SEQ ID NO: 535 - RNA, 17 nt, synthetic construct
  source 1..17: mol_type = other RNA
atcaagcagagaaagcc

SEQ ID NO: 536 - RNA, 17 nt, synthetic construct
  source 1..17: mol_type = other RNA
tagtttggagatggcag

SEQ ID NO: 537 - RNA, 17 nt, synthetic construct
  source 1..17: mol_type = other RNA
gagatggcagtttcctt

SEQ ID NO: 538 - RNA, 17 nt, synthetic construct
  source 1..17: mol_type = other RNA
ccttagtaaccacaggt

SEQ ID NO: 539 - RNA, 17 nt, synthetic construct
  source 1..17: mol_type = other RNA
atatcctcaaggtcacc

SEQ ID NO: 540 - RNA, 17 nt, synthetic construct
  source 1..17: mol_type = other RNA
cttagtaaccacaggtt

SEQ ID NO: 541 - RNA, 17 nt, synthetic construct
  source 1..17: mol_type = other RNA
tgtccaagcccggttga

SEQ ID NO: 542 - RNA, 17 nt, synthetic construct
  source 1..17: mol_type = other RNA
ctagtttggagatggca

SEQ ID NO: 543 - RNA, 17 nt, synthetic construct
  source 1..17: mol_type = other RNA
aaatctgccagagcagg

SEQ ID NO: 544 - RNA, 17 nt, synthetic construct
  source 1..17: mol_type = other RNA
taacttgatcaagcaga

SEQ ID NO: 545 - RNA, 17 nt, synthetic construct
  source 1..17: mol_type = other RNA
gtaaccacaggttgtgt

SEQ ID NO: 546 - RNA, 17 nt, synthetic construct
  source 1..17: mol_type = other RNA
tgtcaccagagtaacag

SEQ ID NO: 547 - RNA, 17 nt, synthetic construct
  source 1..17: mol_type = other RNA
ataacttgatcaagcag

SEQ ID NO: 548 - RNA, 17 nt, synthetic construct
  source 1..17: mol_type = other RNA
ggagatggcagtttcct

SEQ ID NO: 549 - RNA, 17 nt, synthetic construct
  source 1..17: mol_type = other RNA
ctctggtgacacaacct

SEQ ID NO: 550 - RNA, 17 nt, synthetic construct
  source 1..17: mol_type = other RNA
atgttggaggtacctgc

SEQ ID NO: 551 - RNA, 17 nt, synthetic construct
  source 1..17: mol_type = other RNA
ctctgcttgatcaagtt

SEQ ID NO: 552 - RNA, 17 nt, synthetic construct
  source 1..17: mol_type = other RNA
tctgcttgatcaagtta

SEQ ID NO: 553 - RNA, 17 nt, synthetic construct
  source 1..17: mol_type = other RNA
cttgatgttggaggtac

SEQ ID NO: 554 - RNA, 17 nt, synthetic construct
  source 1..17: mol_type = other RNA
aaccgggcttggacaga

SEQ ID NO: 555 - RNA, 17 nt, synthetic construct
  source 1..17: mol_type = other RNA
aggatatcaacgagatg

SEQ ID NO: 556 - RNA, 17 nt, synthetic construct
  source 1..17: mol_type = other RNA
gaggtacctgctctggc

SEQ ID NO: 557 - RNA, 17 nt, synthetic construct
  source 1..17: mol_type = other RNA
caaccgggcttggacag

SEQ ID NO: 558 - RNA, 17 nt, synthetic construct
  source 1..17: mol_type = other RNA
ctaaggaaactgccatc

SEQ ID NO: 559 - RNA, 17 nt, synthetic construct
  source 1..17: mol_type = other RNA
taaaatcacagagggtg

SEQ ID NO: 560 - RNA, 17 nt, synthetic construct
  source 1..17: mol_type = other RNA
gacagaacttaccgact

SEQ ID NO: 561 - RNA, 17 nt, synthetic construct
  source 1..17: mol_type = other RNA
ccgactggctttctctg

SEQ ID NO: 562 - RNA, 17 nt, synthetic construct
  source 1..17: mol_type = other RNA
ccgccttccactcagag

SEQ ID NO: 563 - RNA, 17 nt, synthetic construct
  source 1..17: mol_type = other RNA
ccctcagctcttgaagt

SEQ ID NO: 564 - RNA, 17 nt, synthetic construct
  source 1..17: mol_type = other RNA
aagtaaacggtttaccg

SEQ ID NO: 565 - RNA, 17 nt, synthetic construct
  source 1..17: mol_type = other RNA
accgccttccactcaga

SEQ ID NO: 566 - RNA, 17 nt, synthetic construct
  source 1..17: mol_type = other RNA
gccctcagctcttgaag

SEQ ID NO: 567 - RNA, 17 nt, synthetic construct
  source 1..17: mol_type = other RNA
cactcagagctcagatc

SEQ ID NO: 568 - RNA, 17 nt, synthetic construct
  source 1..17: mol_type = other RNA
cttcaagagctgagggc

SEQ ID NO: 569 - RNA, 17 nt, synthetic construct
  source 1..17: mol_type = other RNA
acttcaagagctgaggg

SEQ ID NO: 570 - RNA, 17 nt, synthetic construct
  source 1..17: mol_type = other RNA
aagagctgagggcaaag

SEQ ID NO: 571 - RNA, 17 nt, synthetic construct
  source 1..17: mol_type = other RNA
tcctgtagaatactggc

SEQ ID NO: 572 - RNA, 17 nt, synthetic construct
  source 1..17: mol_type = other RNA
gaggattgctgaattat

SEQ ID NO: 573 - RNA, 17 nt, synthetic construct
  source 1..17: mol_type = other RNA
gccgctgcccaatgcca

SEQ ID NO: 574 - RNA, 17 nt, synthetic construct
  source 1..17: mol_type = other RNA
ttccccagttgcattca

SEQ ID NO: 575 - RNA, 17 nt, synthetic construct
  source 1..17: mol_type = other RNA
tgaggattgctgaatta

SEQ ID NO: 576 - RNA, 17 nt, synthetic construct
  source 1..17: mol_type = other RNA
ccgctgcccaatgccat

SEQ ID NO: 577 - RNA, 17 nt, synthetic construct
  source 1..17: mol_type = other RNA
aggattgctgaattatt

SEQ ID NO: 578 - RNA, 17 nt, synthetic construct
  source 1..17: mol_type = other RNA
ccaatttttcctgtaga

SEQ ID NO: 579 - RNA, 17 nt, synthetic construct
  source 1..17: mol_type = other RNA
ttcctgtagaatactgg

SEQ ID NO: 580 - RNA, 17 nt, synthetic construct
  source 1..17: mol_type = other RNA
gcagacctcctgccacc

SEQ ID NO: 581 - RNA, 17 nt, synthetic construct
  source 1..17: mol_type = other RNA
cattcaatgttctgaca

SEQ ID NO: 582 - RNA, 17 nt, synthetic construct
  source 1..17: mol_type = other RNA
tgacaacagtttgccgc

SEQ ID NO: 583 - RNA, 17 nt, synthetic construct
  source 1..17: mol_type = other RNA
cttccccagttgcattc

SEQ ID NO: 584 - RNA, 17 nt, synthetic construct
  source 1..17: mol_type = other RNA
cctgtagaatactggca

SEQ ID NO: 585 - RNA, 17 nt, synthetic construct
  source 1..17: mol_type = other RNA
ctgaattatttcttccc

SEQ ID NO: 586 - RNA, 17 nt, synthetic construct
  source 1..17: mol_type = other RNA
ctgtagaatactggcat

SEQ ID NO: 587 - RNA, 17 nt, synthetic construct
  source 1..17: mol_type = other RNA
ttgaggattgctgaatt

SEQ ID NO: 588 - RNA, 17 nt, synthetic construct
  source 1..17: mol_type = other RNA
aatgttctgacaacagt

SEQ ID NO: 589 - RNA, 17 nt, synthetic construct
  source 1..17: mol_type = other RNA
cccagttgcattcaatg

SEQ ID NO: 590 - RNA, 17 nt, synthetic construct
  source 1..17: mol_type = other RNA
cagacctcctgccaccg

SEQ ID NO: 591 - RNA, 17 nt, synthetic construct
  source 1..17: mol_type = other RNA
tttcttccccagttgca

SEQ ID NO: 592 - RNA, 17 nt, synthetic construct
  source 1..17: mol_type = other RNA
aggcttcccaatttttc

SEQ ID NO: 593 - RNA, 17 nt, synthetic construct
  source 1..17: mol_type = other RNA
ggaagcctgaatctgcg

SEQ ID NO: 594 - RNA, 17 nt, synthetic construct
  source 1..17: mol_type = other RNA
agcaatcctcaaaaaca

SEQ ID NO: 595 - RNA, 17 nt, synthetic construct
  source 1..17: mol_type = other RNA
tcagaacattgaatgca

SEQ ID NO: 596 - RNA, 17 nt, synthetic construct
  source 1..17: mol_type = other RNA
aatgcaactggggaaga

SEQ ID NO: 597 - RNA, 17 nt, synthetic construct
  source 1..17: mol_type = other RNA
ggcagcggcaaactgtt

SEQ ID NO: 598 - RNA, 17 nt, synthetic construct
  source 1..17: mol_type = other RNA
tacaggaaaaattggga

SEQ ID NO: 599 - RNA, 17 nt, synthetic construct
  source 1..17: mol_type = other RNA
gccactgattaaatatc

SEQ ID NO: 600 - RNA, 17 nt, synthetic construct
  source 1..17: mol_type = other RNA
gtgtctttctgagaaac

SEQ ID NO: 601 - RNA, 17 nt, synthetic construct
  source 1..17: mol_type = other RNA
tcaacagatctgtcaaa

SEQ ID NO: 602 - RNA, 17 nt, synthetic construct
  source 1..17: mol_type = other RNA
gtatttagcatgttccc

SEQ ID NO: 603 - RNA, 17 nt, synthetic construct
  source 1..17: mol_type = other RNA
tatttagcatgttccca

SEQ ID NO: 604 - RNA, 17 nt, synthetic construct
  source 1..17: mol_type = other RNA
tgttagccactgattaa

SEQ ID NO: 605 - RNA, 17 nt, synthetic construct
  source 1..17: mol_type = other RNA
tgagaaactgttcagct

SEQ ID NO: 606 - RNA, 17 nt, synthetic construct
  source 1..17: mol_type = other RNA
agcatgttcccaattct

SEQ ID NO: 607 - RNA, 17 nt, synthetic construct
  source 1..17: mol_type = other RNA
tatcataatgaaaacgc

SEQ ID NO: 608 - RNA, 17 nt, synthetic construct
  source 1..17: mol_type = other RNA
ctgagaaactgttcagc

SEQ ID NO: 609 - RNA, 17 nt, synthetic construct
  source 1..17: mol_type = other RNA
ccaattctcaggaattt

SEQ ID NO: 610 - RNA, 17 nt, synthetic construct
  source 1..17: mol_type = other RNA
tgtctttctgagaaact

SEQ ID NO: 611 - RNA, 17 nt, synthetic construct
  source 1..17: mol_type = other RNA
tcaggaatttgtgtctt

SEQ ID NO: 612 - RNA, 17 nt, synthetic construct
  source 1..17: mol_type = other RNA
gcatgttcccaattctc

SEQ ID NO: 613 - RNA, 17 nt, synthetic construct
  source 1..17: mol_type = other RNA
agcttctgttagccact

SEQ ID NO: 614 - RNA, 17 nt, synthetic construct
  source 1..17: mol_type = other RNA
tcagaaagacacaaatt

SEQ ID NO: 615 - RNA, 17 nt, synthetic construct
  source 1..17: mol_type = other RNA
aatcagtggctaacaga

SEQ ID NO: 616 - RNA, 17 nt, synthetic construct
  source 1..17: mol_type = other RNA
agaaatggcggcgtttt

SEQ ID NO: 617 - RNA, 17 nt, synthetic construct
  source 1..17: mol_type = other RNA
atcagtggctaacagaa

SEQ ID NO: 618 - RNA, 17 nt, synthetic construct
  source 1..17: mol_type = other RNA
ggaacatgctaaataca

SEQ ID NO: 619 - RNA, 17 nt, synthetic construct
  source 1..17: mol_type = other RNA
ctgagaattgggaacat

SEQ ID NO: 620 - RNA, 17 nt, synthetic construct
  source 1..17: mol_type = other RNA
ctcagaaagacacaaat

SEQ ID NO: 621 - RNA, 13 nt, synthetic construct
  source 1..13: mol_type = other RNA
  modified_base 11: mod_base = OTHER; note = 2'-O-Methyl cytidine
  modified_base 12: mod_base = OTHER; note = 2'-O-Methyl adenosine
  modified_base 13: mod_base = OTHER; note = 2'-O-Methyl guanosine
tggtatcttacag

SEQ ID NO: 622 - RNA, 20 nt, synthetic construct
  source 1..20: mol_type = other RNA
aaattgtatttatcttcagc

SEQ ID NO: 623 - RNA, 20 nt, synthetic construct
  source 1..20: mol_type = other RNA
aaaaaacaaatcaaagactt

SEQ ID NO: 624 - RNA, 20 nt, synthetic construct
  source 1..20: mol_type = other RNA
cctgcaggtaaaagcatatg

SEQ ID NO: 625 - RNA, 20 nt, synthetic construct
  source 1..20: mol_type = other RNA
ccctacctcttttttctgtc

SEQ ID NO: 626 - RNA, 20 nt, synthetic construct
  source 1..20: mol_type = other RNA
aataagtaatgtgtatgctt

SEQ ID NO: 627 - RNA, 20 nt, synthetic construct
  source 1..20: mol_type = other RNA
attaacactttgaagataat

SEQ ID NO: 628 - RNA, 20 nt, synthetic construct
  source 1..20: mol_type = other RNA
gcctgggtgacagtgagact

SEQ ID NO: 629 - RNA, 20 nt, synthetic construct
  source 1..20: mol_type = other RNA
ttttaaaaaggtatctttga

SEQ ID NO: 630 - RNA, 20 nt, synthetic construct
  source 1..20: mol_type = other RNA
acgtgattttctgttaataa

SEQ ID NO: 631 - RNA, 20 nt, synthetic construct
  source 1..20: mol_type = other RNA
cattaaacatcattaaatta

SEQ ID NO: 632 - RNA, 20 nt, synthetic construct
  source 1..20: mol_type = other RNA
atactaaccttggtttctgt

SEQ ID NO: 633 - RNA, 20 nt, synthetic construct
  source 1..20: mol_type = other RNA
aaaaggtatctttgatacta

SEQ ID NO: 634 - RNA, 20 nt, synthetic construct
  source 1..20: mol_type = other RNA
tcaaatgtaaccagtatttt

SEQ ID NO: 635 - RNA, 20 nt, synthetic construct
  source 1..20: mol_type = other RNA
atgtaaagttattaacagaa

SEQ ID NO: 636 - RNA, 20 nt, synthetic construct
  source 1..20: mol_type = other RNA
ttttcttagagacagagtct

SEQ ID NO: 637 - RNA, 20 nt, synthetic construct
  source 1..20: mol_type = other RNA
tatatttattttcttagaga

SEQ ID NO: 638 - RNA, 20 nt, synthetic construct
  source 1..20: mol_type = other RNA
aaataaaatactggttacat

SEQ ID NO: 639 - RNA, 20 nt, synthetic construct
  source 1..20: mol_type = other RNA
atgatgtttaatgtaaagtt

SEQ ID NO: 640 - RNA, 20 nt, synthetic construct
  source 1..20: mol_type = other RNA
ataaaattataccatagatt

SEQ ID NO: 641 - RNA, 20 nt, synthetic construct
  source 1..20: mol_type = other RNA
atggcaaatattagtttctg

SEQ ID NO: 642 - RNA, 20 nt, synthetic construct
  source 1..20: mol_type = other RNA
aatgtaacttccaaacgtta

SEQ ID NO: 643 - RNA, 20 nt, synthetic construct
  source 1..20: mol_type = other RNA
aaaaattattcattgtgtta

SEQ ID NO: 644 - RNA, 20 nt, synthetic construct
  source 1..20: mol_type = other RNA
tgttgcttatttaaaaaatt

SEQ ID NO: 645 - RNA, 20 nt, synthetic construct
  source 1..20: mol_type = other RNA
cttttttgatggcaaatatt

SEQ ID NO: 646 - RNA, 20 nt, synthetic construct
  source 1..20: mol_type = other RNA
acagaaactaatatttgcca

SEQ ID NO: 647 - RNA, 20 nt, synthetic construct
  source 1..20: mol_type = other RNA
aaatgtctcctccagactag

SEQ ID NO: 648 - RNA, 20 nt, synthetic construct
  source 1..20: mol_type = other RNA
ccatcaaaaaagcaaagaat

SEQ ID NO: 649 - RNA, 20 nt, synthetic construct
  source 1..20: mol_type = other RNA
aataagcaacataaatgtga

SEQ ID NO: 650 - RNA, 20 nt, synthetic construct
  source 1..20: mol_type = other RNA
tttttccttttattctagtt

SEQ ID NO: 651 - RNA, 20 nt, synthetic construct
  source 1..20: mol_type = other RNA
ttctagttgaaagaattcag

SEQ ID NO: 652 - RNA, 20 nt, synthetic construct
  source 1..20: mol_type = other RNA
ctactatatatttatttttc

SEQ ID NO: 653 - RNA, 20 nt, synthetic construct
  source 1..20: mol_type = other RNA
gaagttacatttaaaatgtc

SEQ ID NO: 654 - RNA, 20 nt, synthetic construct
  source 1..20: mol_type = other RNA
tatccttgattatacttagg

SEQ ID NO: 655 - RNA, 20 nt, synthetic construct
  source 1..20: mol_type = other RNA
cattaatttatatccttgat

SEQ ID NO: 656 - RNA, 20 nt, synthetic construct
  source 1..20: mol_type = other RNA
aagaaaaacttctgccaact

SEQ ID NO: 657 - RNA, 20 nt, synthetic construct
  source 1..20: mol_type = other RNA
tcattttttctcataccttc

SEQ ID NO: 658 - RNA, 20 nt, synthetic construct
  source 1..20: mol_type = other RNA
ttatttgcattaatttatat

SEQ ID NO: 659 - RNA, 20 nt, synthetic construct
  source 1..20: mol_type = other RNA
ctgagagagaaacagttgcc

SEQ ID NO: 660 - RNA, 20 nt, synthetic construct
  source 1..20: mol_type = other RNA
ctctcctagaccatttccca

SEQ ID NO: 661 - RNA, 20 nt, synthetic construct
  source 1..20: mol_type = other RNA
aaatgaagattttccaccaa

SEQ ID NO: 662 - RNA, 20 nt, synthetic construct
  source 1..20: mol_type = other RNA
agccaaactcttattcatga

SEQ ID NO: 663 - RNA, 20 nt, synthetic construct
  source 1..20: mol_type = other RNA
ggtttttgcaaaaaggaaaa

SEQ ID NO: 664 - RNA, 20 nt, synthetic construct
  source 1..20: mol_type = other RNA
gcaatacatggtagaaaatg

SEQ ID NO: 665 - RNA, 20 nt, synthetic construct
  source 1..20: mol_type = other RNA
tttagcaatacatggtagaa

SEQ ID NO: 666 - RNA, 20 nt, synthetic construct
  source 1..20: mol_type = other RNA
attgaagagtaacaatttga

SEQ ID NO: 667 - RNA, 20 nt, synthetic construct
  source 1..20: mol_type = other RNA
caaaaaggaaaaaagaagaa

SEQ ID NO: 668 - RNA, 20 nt, synthetic construct
  source 1..20: mol_type = other RNA
gcttgtgtttctaatttttc

SEQ ID NO: 669 - RNA, 20 nt, synthetic construct
  source 1..20: mol_type = other RNA
gctcaaattgttactcttca

SEQ ID NO: 670 - RNA, 20 nt, synthetic construct
  source 1..20: mol_type = other RNA
acttattgttattgaaattg

SEQ ID NO: 671 - RNA, 20 nt, synthetic construct
  source 1..20: mol_type = other RNA
gctcctactcagactgttac

SEQ ID NO: 672 - RNA, 20 nt, synthetic construct
  source 1..20: mol_type = other RNA
caaaaacccaaaatatttta

SEQ ID NO: 673 - RNA, 20 nt, synthetic construct
  source 1..20: mol_type = other RNA
cacagtcaataacacaaagg

SEQ ID NO: 674 - RNA, 20 nt, synthetic construct
  source 1..20: mol_type = other RNA
aacaaatagctagagccaaa

SEQ ID NO: 675 - RNA, 20 nt, synthetic construct
  source 1..20: mol_type = other RNA
aatcagagtcaatttccaag

SEQ ID NO: 676 - RNA, 20 nt, synthetic construct
  source 1..20: mol_type = other RNA
tctctatctttagaattgaa

SEQ ID NO: 677 - RNA, 20 nt, synthetic construct
  source 1..20: mol_type = other RNA
gaattgaaacaaattttctc

SEQ ID NO: 678 - RNA, 20 nt, synthetic construct
  source 1..20: mol_type = other RNA
gctctagctatttgttcaaa

SEQ ID NO: 679 - RNA, 20 nt, synthetic construct
  source 1..20: mol_type = other RNA
tttcaattctaaagatagag

SEQ ID NO: 680 - RNA, 20 nt, synthetic construct
  source 1..20: mol_type = other RNA
ttcaaaagtgcaactatgaa

SEQ ID NO: 681 - RNA, 20 nt, synthetic construct
  source 1..20: mol_type = other RNA
tgttattgactgtgcaaaaa

SEQ ID NO: 682 - RNA, 20 nt, synthetic construct
  source 1..20: mol_type = other RNA
gtgggtagaatttcttttaa

SEQ ID NO: 683 - RNA, 20 nt, synthetic construct
  source 1..20: mol_type = other RNA
gtgaatatattattggattt

SEQ ID NO: 684 - RNA, 20 nt, synthetic construct
  source 1..20: mol_type = other RNA
acagaaaagcatacacatta

SEQ ID NO: 685 - RNA, 20 nt, synthetic construct
  source 1..20: mol_type = other RNA
cttctaaattaactttagtg

SEQ ID NO: 686 - RNA, 20 nt, synthetic construct
  source 1..20: mol_type = other RNA
aagataattcatgaacatct

SEQ ID NO: 687 - RNA, 20 nt, synthetic construct
  source 1..20: mol_type = other RNA
gccaaccaccctacaaatat

SEQ ID NO: 688 - RNA, 20 nt, synthetic construct
  source 1..20: mol_type = other RNA
aaggaattataattatttta

SEQ ID NO: 689 - RNA, 20 nt, synthetic construct
  source 1..20: mol_type = other RNA
gaagtaaaatataatagaaa

SEQ ID NO: 690 - RNA, 20 nt, synthetic construct
  source 1..20: mol_type = other RNA
tagggtggttggctaaaata

SEQ ID NO: 691 - RNA, 20 nt, synthetic construct
  source 1..20: mol_type = other RNA
aaagaaattctacccactaa

SEQ ID NO: 692 - RNA, 20 nt, synthetic construct
  source 1..20: mol_type = other RNA
ctgctgttgattaatggttg

SEQ ID NO: 693 - RNA, 20 nt, synthetic construct
  source 1..20: mol_type = other RNA
atgttagtgcctttcaccct

SEQ ID NO: 694 - RNA, 20 nt, synthetic construct
  source 1..20: mol_type = other RNA
gtatatgaaaaaaagaaata

SEQ ID NO: 695 - RNA, 20 nt, synthetic construct
  source 1..20: mol_type = other RNA
agctttctttagaagaatat

SEQ ID NO: 696 - RNA, 20 nt, synthetic construct
  source 1..20: mol_type = other RNA
gaagaatatttcatgagaga

SEQ ID NO: 697 - RNA, 20 nt, synthetic construct
  source 1..20: mol_type = other RNA
gcagactttttaagctttct

SEQ ID NO: 698 - RNA, 20 nt, synthetic construct
  source 1..20: mol_type = other RNA
tttctttttttcatatacta

SEQ ID NO: 699 - RNA, 20 nt, synthetic construct
  source 1..20: mol_type = other RNA
agaaaagattaaacagtgtg

SEQ ID NO: 700 - RNA, 20 nt, synthetic construct
  source 1..20: mol_type = other RNA
tttgagaaaagattaaacag

SEQ ID NO: 701 - RNA, 20 nt, synthetic construct
  source 1..20: mol_type = other RNA
ttttgcctttttggtatctt

SEQ ID NO: 702 - RNA, 20 nt, synthetic construct
  source 1..20: mol_type = other RNA
tatttctttctttgccagta

SEQ ID NO: 703 - RNA, 20 nt, synthetic construct
  source 1..20: mol_type = other RNA
gtatcttacaggaactccag

SEQ ID NO: 704 - RNA, 20 nt, synthetic construct
  source 1..20: mol_type = other RNA
cctttttggtatcttacagg

SEQ ID NO: 705 - RNA, 20 nt, synthetic construct
  source 1..20: mol_type = other RNA
ccagtacaactgcatgtggt

SEQ ID NO: 706 - RNA, 20 nt, synthetic construct
  source 1..20: mol_type = other RNA
gatatgggcatgtcagtttc

SEQ ID NO: 707 - RNA, 20 nt, synthetic construct
  source 1..20: mol_type = other RNA
atcttttctcaaataaaaag

SEQ ID NO: 708 - RNA, 20 nt, synthetic construct
  source 1..20: mol_type = other RNA
agaagttaaagagtccagat

SEQ ID NO: 709 - RNA, 20 nt, synthetic construct
  source 1..20: mol_type = other RNA
atttgttttttcgaaattgt

SEQ ID NO: 710 - RNA, 20 nt, synthetic construct
  source 1..20: mol_type = other RNA
actgttgttgtcatcattat

SEQ ID NO: 711 - RNA, 20 nt, synthetic construct
  source 1..20: mol_type = other RNA
ttttttcgaaattgtattta

SEQ ID NO: 712 - RNA, 20 nt, synthetic construct
  source 1..20: mol_type = other RNA
acttcttaaagatcaggttc

SEQ ID NO: 713 - RNA, 20 nt, synthetic construct
  source 1..20: mol_type = other RNA
tcttcagcacatctggactc

SEQ ID NO: 714 - RNA, 20 nt, synthetic construct
  source 1..20: mol_type = other RNA
cagagtttagtttcaagtaa

SEQ ID NO: 715 - RNA, 20 nt, synthetic construct
  source 1..20: mol_type = other RNA
gtttcaagtaacaaataaaa

SEQ ID NO: 716 - RNA, 20 nt, synthetic construct
  source 1..20: mol_type = other RNA
gttatactgacaaagatatc

SEQ ID NO: 717 - RNA, 20 nt, synthetic construct
  source 1..20: mol_type = other RNA
tttgttacttgaaactaaac

SEQ ID NO: 718 - RNA, 20 nt, synthetic construct
  source 1..20: mol_type = other RNA
ttacttgaaactaaactctg

SEQ ID NO: 719 - RNA, 20 nt, synthetic construct
  source 1..20: mol_type = other RNA
acagatctgttgagaaatgg

SEQ ID NO: 720 - RNA, 20 nt, synthetic construct
  source 1..20: mol_type = other RNA
cataatccatctatttttct

SEQ ID NO: 721 - RNA, 20 nt, synthetic construct
  source 1..20: mol_type = other RNA
cctgcaggcgatttgacaga

SEQ ID NO: 722 - RNA, 20 nt, synthetic construct
  source 1..20: mol_type = other RNA
aaatgttgtgtgtacatgct

SEQ ID NO: 723 - RNA, 20 nt, synthetic construct
  source 1..20: mol_type = other RNA
tcagtataaccaaaaaatat

SEQ ID NO: 724 - RNA, 20 nt, synthetic construct
  source 1..20: mol_type = other RNA
acatttcattcaactgttgc

SEQ ID NO: 725 - RNA, 20 nt, synthetic construct
  source 1..20: mol_type = other RNA
gattgcatctactgtatagg

SEQ ID NO: 726 - RNA, 20 nt, synthetic construct
  source 1..20: mol_type = other RNA
taacttgatcaagcagagaa

SEQ ID NO: 727 - RNA, 20 nt, synthetic construct
  source 1..20: mol_type = other RNA
gagatggcagtttccttagt

SEQ ID NO: 728 - RNA, 20 nt, synthetic construct
  source 1..20: mol_type = other RNA
ccctcagctcttgaagtaaa

SEQ ID NO: 729 - RNA, 20 nt, synthetic construct
  source 1..20: mol_type = other RNA
ccgccttccactcagagctc

SEQ ID NO: 730 - RNA, 20 nt, synthetic construct
  source 1..20: mol_type = other RNA
cttcaagagctgagggcaaa

SEQ ID NO: 731 - RNA, 20 nt, synthetic construct
  source 1..20: mol_type = other RNA
cagacctcctgccaccgcag

SEQ ID NO: 732 - RNA, 20 nt, synthetic construct
  source 1..20: mol_type = other RNA
ccgctgcccaatgccatcct

SEQ ID NO: 733 - RNA, 20 nt, synthetic construct
  source 1..20: mol_type = other RNA
aggattgctgaattatttct

SEQ ID NO: 734 - RNA, 20 nt, synthetic construct
  source 1..20: mol_type = other RNA
gcatgttcccaattctcagg

SEQ ID NO: 735 - RNA, 20 nt, synthetic construct
  source 1..20: mol_type = other RNA
tatcataatgaaaacgccgc

SEQ ID NO: 736 - RNA, 20 nt, synthetic construct
  source 1..20: mol_type = other RNA
tgtctttctgagaaactgtt

SEQ ID NO: 737 - RNA, 20 nt, synthetic construct
  source 1..20: mol_type = other RNA
tatttagcatgttcccaatt

SEQ ID NO: 738 - RNA, 20 nt, synthetic construct
  source 1..20: mol_type = other RNA
atcagtggctaacagaagct

SEQ ID NO: 739 - RNA, 24 nt, synthetic construct
  source 1..24: mol_type = other RNA
cctgattgctcgctgcggcgagac

SEQ ID NO: 740 - RNA, 20 nt, synthetic construct
  source 1..20: mol_type = other RNA
attgctccttacgaggagac

SEQ ID NO: 741 - RNA, 21 nt, synthetic construct
  source 1..21: mol_type = other RNA
gattgctccttacgaggagac

SEQ ID NO: 742 - RNA, 24 nt, synthetic construct
  source 1..24: mol_type = other RNA
atagattgctccttacgaggagac

SEQ ID NO: 743 - RNA, 78 nt, synthetic construct
  source 1..78: mol_type = other RNA
tggggcagttggttgcccttagcctgaggcatttattgcactcgggaagtaccatttctcagaaatggtacatccaac

SEQ ID NO: 744 - RNA, 141 nt, synthetic construct
  source 1..141: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaac

SEQ ID NO: 745 - RNA, 17 nt, synthetic construct
  source 1..17: mol_type = other RNA
gaatgaaggaatgcaac

SEQ ID NO: 746 - RNA, 140 nt, synthetic construct
  source 1..140: mol_type = other RNA
accgcttcaccaagtgctgtcccttagggattagcacttgagtgaaggtgggctgcttgcatcagcctaatgtcgagaagtgctttcttcggaaagtaaccctcgaaacaaattcatttgaaagaatgaaggaatgcaac

SEQ ID NO: 747 - RNA, 119 nt, synthetic construct
  source 1..119: mol_type = other RNA
accgcttcaccaagtgctgtcccttagggattagcacttgagtgaaggtgggctgcttgcatcagcctaatgtcgagaagtgctttcttcggaaagtaaccctcgaaacaaattcattt

SEQ ID NO: 748 - RNA, 49 nt, synthetic construct
  source 1..49: mol_type = other RNA
cctgattgctcgctgcggcgagactactgacaaagatatcactctttta

SEQ ID NO: 749 - RNA, 49 nt, synthetic construct
  source 1..49: mol_type = other RNA
cctgattgctcgctgcggcgagaccctgcatttgcagagtttagtttta

SEQ ID NO: 750 - RNA, 49 nt, synthetic construct
  source 1..49: mol_type = other RNA
cctgattgctcgctgcggcgagaccttgaaactaaactctgcaatttta

SEQ ID NO: 751 - RNA, 49 nt, synthetic construct
  source 1..49: mol_type = other RNA
cctgattgctcgctgcggcgagactacataatccatctattttttttta

SEQ ID NO: 752 - RNA, 49 nt, synthetic construct
  source 1..49: mol_type = other RNA
cctgattgctcgctgcggcgagactgaagggtgatggaaattactttta

SEQ ID NO: 753 - RNA, 49 nt, synthetic construct
  source 1..49: mol_type = other RNA
cctgattgctcgctgcggcgagacaagagtccagatgtgctgaatttta

SEQ ID NO: 754 - RNA, 49 nt, synthetic construct
  source 1..49: mol_type = other RNA
cctgattgctcgctgcggcgagactcatcattatattactagaatttta

SEQ ID NO: 755 - RNA, 49 nt, synthetic construct
  source 1..49: mol_type = other RNA
cctgattgctcgctgcggcgagactttcgaaattgtatttatcttttta

SEQ ID NO: 756 - RNA, 49 nt, synthetic construct
  source 1..49: mol_type = other RNA
cctgattgctcgctgcggcgagacttgtcatcattatattactatttta

SEQ ID NO: 757 - RNA, 49 nt, synthetic construct
  source 1..49: mol_type = other RNA
cctgattgctcgctgcggcgagaccatagggaaattttcacatgtttta

SEQ ID NO: 758 - RNA, 49 nt, synthetic construct
  source 1..49: mol_type = other RNA
cctgattgctcgctgcggcgagacctgtaagataccaaaaaggctttta

SEQ ID NO: 759 - RNA, 49 nt, synthetic construct
  source 1..49: mol_type = other RNA
cctgattgctcgctgcggcgagactgcctttttggtatcttacatttta

SEQ ID NO: 760 - RNA, 49 nt, synthetic construct
  source 1..49: mol_type = other RNA
cctgattgctcgctgcggcgagactactggcaaagaaagaaatatttta

SEQ ID NO: 761 - RNA, 49 nt, synthetic construct
  source 1..49: mol_type = other RNA
cctgattgctcgctgcggcgagacaatcttttctcaaataaaaatttta

SEQ ID NO: 762 - RNA, 49 nt, synthetic construct
  source 1..49: mol_type = other RNA
cctgattgctcgctgcggcgagacattaatggttgataggttcttttta

SEQ ID NO: 763 - RNA, 49 nt, synthetic construct
  source 1..49: mol_type = other RNA
cctgattgctcgctgcggcgagacgtgcctttcaccctgcttattttta

SEQ ID NO: 764 - RNA, 49 nt, synthetic construct
  source 1..49: mol_type = other RNA
cctgattgctcgctgcggcgagactcattcctattagatctgtctttta

SEQ ID NO: 765 - RNA, 49 nt, synthetic construct
  source 1..49: mol_type = other RNA
cctgattgctcgctgcggcgagacataggttctttaatgttagttttta

SEQ ID NO: 766 - RNA, 49 nt, synthetic construct
  source 1..49: mol_type = other RNA
cctgattgctcgctgcggcgagactttaatgttagtgcctttcatttta

SEQ ID NO: 767 - RNA, 49 nt, synthetic construct
  source 1..49: mol_type = other RNA
cctgattgctcgctgcggcgagacatcgaataagtaatgtgtattttta

SEQ ID NO: 768 - RNA, 49 nt, synthetic construct
  source 1..49: mol_type = other RNA
cctgattgctcgctgcggcgagacgaagatctgagctctgagtgtttta

SEQ ID NO: 769 - RNA, 49 nt, synthetic construct
  source 1..49: mol_type = other RNA
cctgattgctcgctgcggcgagacaagaggaagttagaagatcttttta

SEQ ID NO: 770 - RNA, 49 nt, synthetic construct
  source 1..49: mol_type = other RNA
cctgattgctcgctgcggcgagacatgaattatcttcaaagtgttttta

SEQ ID NO: 771 - RNA, 49 nt, synthetic construct
  source 1..49: mol_type = other RNA
cctgattgctcgctgcggcgagacatttagaagtaaaatataattttta

SEQ ID NO: 772 - RNA, 49 nt, synthetic construct
  source 1..49: mol_type = other RNA
cctgattgctcgctgcggcgagacaaaagtgcaactatgaagtgtttta

SEQ ID NO: 773 - RNA, 49 nt, synthetic construct
  source 1..49: mol_type = other RNA
cctgattgctcgctgcggcgagacatctctatctttagaattgatttta

SEQ ID NO: 774 - RNA, 49 nt, synthetic construct
  source 1..49: mol_type = other RNA
cctgattgctcgctgcggcgagaccaattctaaagatagagatatttta

SEQ ID NO: 775 - RNA, 49 nt, synthetic construct
  source 1..49: mol_type = other RNA
cctgattgctcgctgcggcgagaccacttttgaacaaatagctatttta

SEQ ID NO: 776 - RNA, 49 nt, synthetic construct
  source 1..49: mol_type = other RNA
cctgattgctcgctgcggcgagacttgactgtgcaaaaagtctttttta

SEQ ID NO: 777 - RNA, 49 nt, synthetic construct
  source 1..49: mol_type = other RNA
cctgattgctcgctgcggcgagacggctcaaattgttactcttctttta

SEQ ID NO: 778 - RNA, 49 nt, synthetic construct
  source 1..49: mol_type = other RNA
cctgattgctcgctgcggcgagacttgaaattggctctttagcttttta

SEQ ID NO: 779 - RNA, 49 nt, synthetic construct
  source 1..49: mol_type = other RNA
cctgattgctcgctgcggcgagacctcttcaattaaatttgacttttta

SEQ ID NO: 780 - RNA, 49 nt, synthetic construct
  source 1..49: mol_type = other RNA
cctgattgctcgctgcggcgagacttgcaaaaaggaaaaaagaatttta

SEQ ID NO: 781 - RNA, 49 nt, synthetic construct
  source 1..49: mol_type = other RNA
cctgattgctcgctgcggcgagacctaatttttctttttcttcttttta

SEQ ID NO: 782 - RNA, 49 nt, synthetic construct
  source 1..49: mol_type = other RNA
cctgattgctcgctgcggcgagacgctgagagagaaacagttgctttta

SEQ ID NO: 783 - RNA, 49 nt, synthetic construct
  source 1..49: mol_type = other RNA
cctgattgctcgctgcggcgagacctctctcagcaaacacattatttta

SEQ ID NO: 784 - RNA, 49 nt, synthetic construct
  source 1..49: mol_type = other RNA
cctgattgctcgctgcggcgagaccctaagaactggtgggaaattttta

SEQ ID NO: 785 - RNA, 49 nt, synthetic construct
  source 1..49: mol_type = other RNA
cctgattgctcgctgcggcgagacttaggcaactgtttctctcttttta

SEQ ID NO: 786 - RNA, 49 nt, synthetic construct
  source 1..49: mol_type = other RNA
cctgattgctcgctgcggcgagacgcagaagtttttctttaaaatttta

SEQ ID NO: 787 - RNA, 49 nt, synthetic construct
  source 1..49: mol_type = other RNA
cctgattgctcgctgcggcgagacttctttaaaatgaagatttttttta

SEQ ID NO: 788 - RNA, 49 nt, synthetic construct
  source 1..49: mol_type = other RNA
cctgattgctcgctgcggcgagacatcatcctagccataacacatttta

SEQ ID NO: 789 - RNA, 49 nt, synthetic construct
  source 1..49: mol_type = other RNA
cctgattgctcgctgcggcgagacttcatcatcctagccataactttta

SEQ ID NO: 790 - RNA, 49 nt, synthetic construct
  source 1..49: mol_type = other RNA
cctgattgctcgctgcggcgagacaaagaattcagaatcagtggtttta

SEQ ID NO: 791 - RNA, 49 nt, synthetic construct
  source 1..49: mol_type = other RNA
cctgattgctcgctgcggcgagacggaagttacatttaaaatgttttta

SEQ ID NO: 792 - RNA, 49 nt, synthetic construct
  source 1..49: mol_type = other RNA
cctgattgctcgctgcggcgagaccatttaaaatgtctcctccatttta

SEQ ID NO: 793 - RNA, 49 nt, synthetic construct
  source 1..49: mol_type = other RNA
cctgattgctcgctgcggcgagactctcacatttatgttgcttatttta

SEQ ID NO: 794 - RNA, 49 nt, synthetic construct
  source 1..49: mol_type = other RNA
cctgattgctcgctgcggcgagaccttatttaaaaaattattcatttta

SEQ ID NO: 795 - RNA, 49 nt, synthetic construct
  source 1..49: mol_type = other RNA
cctgattgctcgctgcggcgagacttaacagaaaatcacgttaatttta

SEQ ID NO: 796 - RNA, 49 nt, synthetic construct
  source 1..49: mol_type = other RNA
cctgattgctcgctgcggcgagacaagctgaaatgaacagtagatttta

SEQ ID NO: 797 - RNA, 49 nt, synthetic construct
  source 1..49: mol_type = other RNA
cctgattgctcgctgcggcgagacatttcagctttaacgtgatttttta

SEQ ID NO: 798 - RNA, 49 nt, synthetic construct
  source 1..49: mol_type = other RNA
cctgattgctcgctgcggcgagacataactttacattaaacatctttta

SEQ ID NO: 799 - RNA, 49 nt, synthetic construct
  source 1..49: mol_type = other RNA
cctgattgctcgctgcggcgagacgtatcaaagatacctttttatttta

SEQ ID NO: 800 - RNA, 49 nt, synthetic construct
  source 1..49: mol_type = other RNA
cctgattgctcgctgcggcgagaccatttgataaaattataccatttta

SEQ ID NO: 801 - RNA, 49 nt, synthetic construct
  source 1..49: mol_type = other RNA
cctgattgctcgctgcggcgagacaatgtaaagttattaacagatttta

SEQ ID NO: 802 - RNA, 49 nt, synthetic construct
  source 1..49: mol_type = other RNA
cctgattgctcgctgcggcgagacctgtgattttcttttggatttttta

SEQ ID NO: 803 - RNA, 44 nt, synthetic construct
  source 1..44: mol_type = other RNA
cctgattgctcgctgcggcgagacaatcctttaacatttcattc

SEQ ID NO: 804 - RNA, 44 nt, synthetic construct
  source 1..44: mol_type = other RNA
cctgattgctcgctgcggcgagactgaaggtgttcttgtacttc

SEQ ID NO: 805 - RNA, 44 nt, synthetic construct
  source 1..44: mol_type = other RNA
cctgattgctcgctgcggcgagacttgtacttcatcccactgat

SEQ ID NO: 806 - RNA, 44 nt, synthetic construct
  source 1..44: mol_type = other RNA
cctgattgctcgctgcggcgagaccctccggttctgaaggtgtt

SEQ ID NO: 807 - RNA, 44 nt, synthetic construct
  source 1..44: mol_type = other RNA
cctgattgctcgctgcggcgagacaaggattcaacacaatggct

SEQ ID NO: 808 - RNA, 44 nt, synthetic construct
  source 1..44: mol_type = other RNA
cctgattgctcgctgcggcgagacaatgaaatgttaaaggattc

SEQ ID NO: 809 - RNA, 44 nt, synthetic construct
  source 1..44: mol_type = other RNA
cctgattgctcgctgcggcgagactgtcaccagagtaacagtct

SEQ ID NO: 810 - RNA, 44 nt, synthetic construct
  source 1..44: mol_type = other RNA
cctgattgctcgctgcggcgagactgtccaagcccggttgaaat

SEQ ID NO: 811 - RNA, 44 nt, synthetic construct
  source 1..44: mol_type = other RNA
cctgattgctcgctgcggcgagacggagatggcagtttccttag

SEQ ID NO: 812 - RNA, 44 nt, synthetic construct
  source 1..44: mol_type = other RNA
cctgattgctcgctgcggcgagacccttagtaaccacaggttgt

SEQ ID NO: 813 - RNA, 44 nt, synthetic construct
  source 1..44: mol_type = other RNA
cctgattgctcgctgcggcgagacaaatctgccagagcaggtac

SEQ ID NO: 814 - RNA, 44 nt, synthetic construct
  source 1..44: mol_type = other RNA
cctgattgctcgctgcggcgagacatatcctcaaggtcacccac

SEQ ID NO: 815 - RNA, 44 nt, synthetic construct
  source 1..44: mol_type = other RNA
cctgattgctcgctgcggcgagactaaaatcacagagggtgatg

SEQ ID NO: 816 - RNA, 44 nt, synthetic construct
  source 1..44: mol_type = other RNA
cctgattgctcgctgcggcgagacgaggtacctgctctggcaga

SEQ ID NO: 817 - RNA, 44 nt, synthetic construct
  source 1..44: mol_type = other RNA
cctgattgctcgctgcggcgagacctctggtgacacaacctgtg

SEQ ID NO: 818 - RNA, 44 nt, synthetic construct
  source 1..44: mol_type = other RNA
cctgattgctcgctgcggcgagacctaaggaaactgccatctcc

SEQ ID NO: 819 - RNA, 44 nt, synthetic construct
  source 1..44: mol_type = other RNA
cctgattgctcgctgcggcgagacaccgccttccactcagagct

SEQ ID NO: 820 - RNA, 44 nt, synthetic construct
  source 1..44: mol_type = other RNA
cctgattgctcgctgcggcgagacacttcaagagctgagggcaa

SEQ ID NO: 821 - RNA, 44 nt, synthetic construct
  source 1..44: mol_type = other RNA
cctgattgctcgctgcggcgagacttgaggattgctgaattatt

SEQ ID NO: 822 - RNA, 44 nt, synthetic construct
  source 1..44: mol_type = other RNA
cctgattgctcgctgcggcgagacgcagacctcctgccaccgca

SEQ ID NO: 823 - RNA, 44 nt, synthetic construct
  source 1..44: mol_type = other RNA
cctgattgctcgctgcggcgagaccattcaatgttctgacaaca

SEQ ID NO: 824 - RNA, 44 nt, synthetic construct
  source 1..44: mol_type = other RNA
cctgattgctcgctgcggcgagactgacaacagtttgccgctgc

SEQ ID NO: 825 - RNA, 44 nt, synthetic construct
  source 1..44: mol_type = other RNA
cctgattgctcgctgcggcgagacgccgctgcccaatgccatcc

SEQ ID NO: 826 - RNA, 44 nt, synthetic construct
  source 1..44: mol_type = other RNA
cctgattgctcgctgcggcgagactcagaacattgaatgcaact

SEQ ID NO: 827 - RNA, 44 nt, synthetic construct
  source 1..44: mol_type = other RNA
cctgattgctcgctgcggcgagacccaattctcaggaatttgtg

SEQ ID NO: 828 - RNA, 44 nt, synthetic construct
  source 1..44: mol_type = other RNA
cctgattgctcgctgcggcgagacagcttctgttagccactgat

SEQ ID NO: 829 - RNA, 44 nt, synthetic construct
  source 1..44: mol_type = other RNA
cctgattgctcgctgcggcgagacgccactgattaaatatcttt

SEQ ID NO: 830 - RNA, 44 nt, synthetic construct
  source 1..44: mol_type = other RNA
cctgattgctcgctgcggcgagacctcagaaagacacaaattcc

SEQ ID NO: 831 - RNA, 44 nt, synthetic construct
  source 1..44: mol_type = other RNA
cctgattgctcgctgcggcgagacagaaatggcggcgttttcat

SEQ ID NO: 832 - RNA, 43 nt, synthetic construct
  source 1..43: mol_type = other RNA
gattgctccttacgaggagaccctgcaggcgatttgactttta

SEQ ID NO: 833 - RNA, 43 nt, synthetic construct
  source 1..43: mol_type = other RNA
gattgctccttacgaggagacacctgcaggcgatttgatttta

SEQ ID NO: 834 - RNA, 43 nt, synthetic construct
  source 1..43: mol_type = other RNA
gattgctccttacgaggagactacctgcaggcgatttgtttta

SEQ ID NO: 835 - RNA, 43 nt, synthetic construct
  source 1..43: mol_type = other RNA
gattgctccttacgaggagactagagatatagcgtatatttta

SEQ ID NO: 836 - RNA, 43 nt, synthetic construct
  source 1..43: mol_type = other RNA
gattgctccttacgaggagacggttatactgacaaagatttta

SEQ ID NO: 837 - RNA, 43 nt, synthetic construct
  source 1..43: mol_type = other RNA
gattgctccttacgaggagacgacagatctgttgagaatttta

SEQ ID NO: 838 - RNA, 43 nt, synthetic construct
  source 1..43: mol_type = other RNA
gattgctccttacgaggagacctcaacagatctgtcaatttta

SEQ ID NO: 839 - RNA, 43 nt, synthetic construct
  source 1..43: mol_type = other RNA
gattgctccttacgaggagacatccatatgcttttacctttta

SEQ ID NO: 840 - RNA, 43 nt, synthetic construct
  source 1..43: mol_type = other RNA
gattgctccttacgaggagacgttacttgaaactaaactttta

SEQ ID NO: 841 - RNA, 43 nt, synthetic construct
  source 1..43: mol_type = other RNA
gattgctccttacgaggagacgcagagtttagtttcaatttta

SEQ ID NO: 842 - RNA, 43 nt, synthetic construct
  source 1..43: mol_type = other RNA
gattgctccttacgaggagacgttatactgacaaagattttta

SEQ ID NO: 843 - RNA, 43 nt, synthetic construct
  source 1..43: mol_type = other RNA
gattgctccttacgaggagactttggttatactgacaatttta

SEQ ID NO: 844 - RNA, 43 nt, synthetic construct
  source 1..43: mol_type = other RNA
gattgctccttacgaggagaccctgcatttgcagagtttttta

SEQ ID NO: 845 - RNA, 43 nt, synthetic construct
  source 1..43: mol_type = other RNA
gattgctccttacgaggagaccttgaaactaaactctgtttta

SEQ ID NO: 846 - RNA, 43 nt, synthetic construct
  source 1..43: mol_type = other RNA
gattgctccttacgaggagacttcttgatccatatgcttttta

SEQ ID NO: 847 - RNA, 43 nt, synthetic construct
  source 1..43: mol_type = other RNA
gattgctccttacgaggagaccttgatccatatgcttttttta

SEQ ID NO: 848 - RNA, 43 nt, synthetic construct
  source 1..43: mol_type = other RNA
gattgctccttacgaggagactcttgatccatatgctttttta

SEQ ID NO: 849 - RNA, 43 nt, synthetic construct
  source 1..43: mol_type = other RNA
gattgctccttacgaggagactactgacaaagatatcatttta

SEQ ID NO: 850 - RNA, 43 nt, synthetic construct
  source 1..43: mol_type = other RNA
gattgctccttacgaggagacgtcagtataaccaaaaatttta

SEQ ID NO: 851 - RNA, 43 nt, synthetic construct
  source 1..43: mol_type = other RNA
gattgctccttacgaggagacctgcatttgcagagttttttta

SEQ ID NO: 852 - RNA, 43 nt, synthetic construct
  source 1..43: mol_type = other RNA
gattgctccttacgaggagactacataatccatctatttttta

SEQ ID NO: 853 - RNA, 43 nt, synthetic construct
  source 1..43: mol_type = other RNA
gattgctccttacgaggagacaaactaaactctgcaaatttta

SEQ ID NO: 854 - RNA, 43 nt, synthetic construct
  source 1..43: mol_type = other RNA
gattgctccttacgaggagacacagatctgttgagaaatttta

SEQ ID NO: 855 - RNA, 43 nt, synthetic construct
  source 1..43: mol_type = other RNA
gattgctccttacgaggagacgtttcaagtaacaaatatttta

SEQ ID NO: 856 - RNA, 43 nt, synthetic construct
  source 1..43: mol_type = other RNA
gattgctccttacgaggagactgtaaaacagattatagtttta

SEQ ID NO: 857 - RNA, 43 nt, synthetic construct
  source 1..43: mol_type = other RNA
gattgctccttacgaggagacttacttgaaactaaacttttta

SEQ ID NO: 858 - RNA, 43 nt, synthetic construct
  source 1..43: mol_type = other RNA
gattgctccttacgaggagacacataatccatctattttttta

SEQ ID NO: 859 - RNA, 43 nt, synthetic construct
  source 1..43: mol_type = other RNA
gattgctccttacgaggagaccataatccatctatttttttta

SEQ ID NO: 860 - RNA, 43 nt, synthetic construct
  source 1..43: mol_type = other RNA
gattgctccttacgaggagactcagtataaccaaaaaatttta

SEQ ID NO: 861 - RNA, 43 nt, synthetic construct
  source 1..43: mol_type = other RNA
gattgctccttacgaggagacttgatccatatgctttttttta

SEQ ID NO: 862 - RNA, 43 nt, synthetic construct
  source 1..43: mol_type = other RNA
gattgctccttacgaggagaccatcacccttcagaacctttta

SEQ ID NO: 863 - RNA, 43 nt, synthetic construct
  source 1..43: mol_type = other RNA
gattgctccttacgaggagacagcacatctggactctttttta

SEQ ID NO: 864 - RNA, 43 nt, synthetic construct
  source 1..43: mol_type = other RNA
gattgctccttacgaggagacaagagtccagatgtgcttttta

SEQ ID NO: 865 - RNA, 43 nt, synthetic construct
  source 1..43: mol_type = other RNA
gattgctccttacgaggagacatcttcagcacatctggtttta

SEQ ID NO: 866 - RNA, 43 nt, synthetic construct
  source 1..43: mol_type = other RNA
gattgctccttacgaggagacgactgttgttgtcatcatttta

SEQ ID NO: 867 - RNA, 43 nt, synthetic construct
  source 1..43: mol_type = other RNA
gattgctccttacgaggagacaagatcaggttctgaagtttta

SEQ ID NO: 868 - RNA, 43 nt, synthetic construct
  source 1..43: mol_type = other RNA
gattgctccttacgaggagacagaagttaaagagtccatttta

SEQ ID NO: 869 - RNA, 43 nt, synthetic construct
  source 1..43: mol_type = other RNA
gattgctccttacgaggagacaagaagttaaagagtcctttta

SEQ ID NO: 870 - RNA, 43 nt, synthetic construct
  source 1..43: mol_type = other RNA
gattgctccttacgaggagacccttaagataccatttgtttta

SEQ ID NO: 871 - RNA, 43 nt, synthetic construct
  source 1..43: mol_type = other RNA
gattgctccttacgaggagactcttcagcacatctggatttta

SEQ ID NO: 872 - RNA, 43 nt, synthetic construct
  source 1..43: mol_type = other RNA
gattgctccttacgaggagactgaagggtgatggaaattttta

SEQ ID NO: 873 - RNA, 43 nt, synthetic construct
  source 1..43: mol_type = other RNA
gattgctccttacgaggagacacttcttaaagatcaggtttta

SEQ ID NO: 874 - RNA, 43 nt, synthetic construct
  source 1..43: mol_type = other RNA
gattgctccttacgaggagactgactgttgttgtcatctttta

SEQ ID NO: 875 - RNA, 43 nt, synthetic construct
  source 1..43: mol_type = other RNA
gattgctccttacgaggagacactgttgttgtcatcattttta

SEQ ID NO: 876 - RNA, 43 nt, synthetic construct
  source 1..43: mol_type = other RNA
gattgctccttacgaggagactcgaaattgtatttatctttta

SEQ ID NO: 877 - RNA, 43 nt, synthetic construct
  source 1..43: mol_type = other RNA
gattgctccttacgaggagacctagtaatataatgatgtttta

SEQ ID NO: 878 - RNA, 43 nt, synthetic construct
  source 1..43: mol_type = other RNA
gattgctccttacgaggagacaggtaagtctttgattttttta

SEQ ID NO: 879 - RNA, 43 nt, synthetic construct
  source 1..43: mol_type = other RNA
gattgctccttacgaggagacttaaagatcaggttctgtttta

SEQ ID NO: 880 - RNA, 43 nt, synthetic construct
  source 1..43: mol_type = other RNA
gattgctccttacgaggagaccgaaattgtatttatcttttta

SEQ ID NO: 881 - RNA, 43 nt, synthetic construct
  source 1..43: mol_type = other RNA
gattgctccttacgaggagacaacttcttaaagatcagtttta

SEQ ID NO: 882 - RNA, 43 nt, synthetic construct
  source 1..43: mol_type = other RNA
gattgctccttacgaggagacgttttttcgaaattgtatttta

SEQ ID NO: 883 - RNA, 43 nt, synthetic construct
  source 1..43: mol_type = other RNA
gattgctccttacgaggagactatttatcttcagcacatttta

SEQ ID NO: 884 - RNA, 43 nt, synthetic construct
  source 1..43: mol_type = other RNA
gattgctccttacgaggagactagtaatataatgatgatttta

SEQ ID NO: 885 - RNA, 43 nt, synthetic construct
  source 1..43: mol_type = other RNA
gattgctccttacgaggagactcatcattatattactatttta

SEQ ID NO: 886 - RNA, 43 nt, synthetic construct
  source 1..43: mol_type = other RNA
gattgctccttacgaggagacagataccatttgtattttttta

SEQ ID NO: 887 - RNA, 43 nt, synthetic construct
  source 1..43: mol_type = other RNA
gattgctccttacgaggagacctagaaagaaaattatctttta

SEQ ID NO: 888 - RNA, 43 nt, synthetic construct
  source 1..43: mol_type = other RNA
gattgctccttacgaggagacgaaattgtatttatctttttta

SEQ ID NO: 889 - RNA, 43 nt, synthetic construct
  source 1..43: mol_type = other RNA
gattgctccttacgaggagactctttctagtaatataatttta

SEQ ID NO: 890 - RNA, 43 nt, synthetic construct
  source 1..43: mol_type = other RNA
gattgctccttacgaggagacttcgaaattgtatttattttta

SEQ ID NO: 891 - RNA, 43 nt, synthetic construct
  source 1..43: mol_type = other RNA
gattgctccttacgaggagactttcgaaattgtatttatttta

SEQ ID NO: 892 - RNA, 43 nt, synthetic construct
  source 1..43: mol_type = other RNA
gattgctccttacgaggagacttgtcatcattatattatttta

SEQ ID NO: 893 - RNA, 43 nt, synthetic construct
  source 1..43: mol_type = other RNA
gattgctccttacgaggagactttctagtaatataatgtttta

SEQ ID NO: 894 - RNA, 43 nt, synthetic construct
  source 1..43: mol_type = other RNA
gattgctccttacgaggagactattactagaaagaaaatttta

SEQ ID NO: 895 - RNA, 43 nt, synthetic construct
  source 1..43: mol_type = other RNA
gattgctccttacgaggagacgatttgttttttcgaaatttta

SEQ ID NO: 896 - RNA, 43 nt, synthetic construct
  source 1..43: mol_type = other RNA
gattgctccttacgaggagacttttttcgaaattgtattttta

SEQ ID NO: 897 - RNA, 43 nt, synthetic construct
  source 1..43: mol_type = other RNA
gattgctccttacgaggagaccttttgactgttgttgttttta

SEQ ID NO: 898 - RNA, 43 nt, synthetic construct
  source 1..43: mol_type = other RNA
gattgctccttacgaggagacatttgttttttcgaaattttta

SEQ ID NO: 899 - RNA, 43 nt, synthetic construct
  source 1..43: mol_type = other RNA
gattgctccttacgaggagacgccagtacaactgcatgtttta

SEQ ID NO: 900 - RNA, 43 nt, synthetic construct
  source 1..43: mol_type = other RNA
gattgctccttacgaggagacaacagtgtgctaccacatttta

SEQ ID NO: 901 - RNA, 43 nt, synthetic construct
  source 1..43: mol_type = other RNA
gattgctccttacgaggagacggtatcttacaggaacttttta

SEQ ID NO: 902 - RNA, 43 nt, synthetic construct
  source 1..43: mol_type = other RNA
gattgctccttacgaggagactggtatcttacaggaactttta

SEQ ID NO: 903 - RNA, 43 nt, synthetic construct
  source 1..43: mol_type = other RNA
gattgctccttacgaggagacgtatcttacaggaactctttta

SEQ ID NO: 904 - RNA, 43 nt, synthetic construct
  source 1..43: mol_type = other RNA
gattgctccttacgaggagacccagtacaactgcatgttttta

SEQ ID NO: 905 - RNA, 43 nt, synthetic construct
  source 1..43: mol_type = other RNA
gattgctccttacgaggagactttgccagtacaactgctttta

SEQ ID NO: 906 - RNA, 43 nt, synthetic construct
  source 1..43: mol_type = other RNA
gattgctccttacgaggagaccaggaactccaggatggtttta

SEQ ID NO: 907 - RNA, 43 nt, synthetic construct
  source 1..43: mol_type = other RNA
gattgctccttacgaggagacctttgccagtacaactgtttta

SEQ ID NO: 908 - RNA, 43 nt, synthetic construct
  source 1..43: mol_type = other RNA
gattgctccttacgaggagaccacatggagcttttgtatttta

SEQ ID NO: 909 - RNA, 43 nt, synthetic construct
  source 1..43: mol_type = other RNA
gattgctccttacgaggagacccctatgaaactgacattttta

SEQ ID NO: 910 - RNA, 43 nt, synthetic construct
  source 1..43: mol_type = other RNA
gattgctccttacgaggagaccctatgaaactgacatgtttta

SEQ ID NO: 911 - RNA, 43 nt, synthetic construct
  source 1..43: mol_type = other RNA
gattgctccttacgaggagacttggtatcttacaggaatttta

SEQ ID NO: 912 - RNA, 43 nt, synthetic construct
  source 1..43: mol_type = other RNA
gattgctccttacgaggagacacatggagcttttgtattttta

SEQ ID NO: 913 - RNA, 43 nt, synthetic construct
  source 1..43: mol_type = other RNA
gattgctccttacgaggagaccatagggaaattttcactttta

SEQ ID NO: 914 - RNA, 43 nt, synthetic construct
  source 1..43: mol_type = other RNA
gattgctccttacgaggagactcacatggagcttttgttttta

SEQ ID NO: 915 - RNA, 43 nt, synthetic construct
  source 1..43: mol_type = other RNA
gattgctccttacgaggagacgcctttttggtatcttatttta

SEQ ID NO: 916 - RNA, 43 nt, synthetic construct
  source 1..43: mol_type = other RNA
gattgctccttacgaggagaccctttttggtatcttactttta

SEQ ID NO: 917 - RNA, 43 nt, synthetic construct
  source 1..43: mol_type = other RNA
gattgctccttacgaggagacctttctttgccagtacatttta

SEQ ID NO: 918 - RNA, 43 nt, synthetic construct
  source 1..43: mol_type = other RNA
gattgctccttacgaggagacatagggaaattttcacatttta

SEQ ID NO: 919 - RNA, 43 nt, synthetic construct
  source 1..43: mol_type = other RNA
gattgctccttacgaggagactactggcaaagaaagaatttta

SEQ ID NO: 920 - RNA, 43 nt, synthetic construct
  source 1..43: mol_type = other RNA
gattgctccttacgaggagactttctttgccagtacaatttta

SEQ ID NO: 921 - RNA, 43 nt, synthetic construct
  source 1..43: mol_type = other RNA
gattgctccttacgaggagacgagaaaagattaaacagtttta

SEQ ID NO: 922 - RNA, 43 nt, synthetic construct
  source 1..43: mol_type = other RNA
gattgctccttacgaggagactgcctttttggtatctttttta

SEQ ID NO: 923 - RNA, 43 nt, synthetic construct
  source 1..43: mol_type = other RNA
gattgctccttacgaggagacgttttgcctttttggtatttta

SEQ ID NO: 924 - RNA, 43 nt, synthetic construct
  source 1..43: mol_type = other RNA
gattgctccttacgaggagacctcaaataaaaagacattttta

SEQ ID NO: 925 - RNA, 43 nt, synthetic construct
  source 1..43: mol_type = other RNA
gattgctccttacgaggagacagaaaagattaaacagttttta

SEQ ID NO: 926 - RNA, 43 nt, synthetic construct
  source 1..43: mol_type = other RNA
gattgctccttacgaggagacaatcttttctcaaataatttta

SEQ ID NO: 927 - RNA, 43 nt, synthetic construct
  source 1..43: mol_type = other RNA
gattgctccttacgaggagactatttgagaaaagattatttta

SEQ ID NO: 928 - RNA, 43 nt, synthetic construct
  source 1..43: mol_type = other RNA
gattgctccttacgaggagacgtatttctttctttgcctttta

SEQ ID NO: 929 - RNA, 43 nt, synthetic construct
  source 1..43: mol_type = other RNA
gattgctccttacgaggagacatttgagaaaagattaatttta

SEQ ID NO: 930 - RNA, 43 nt, synthetic construct
  source 1..43: mol_type = other RNA
gattgctccttacgaggagactcaaataaaaagacatgtttta

SEQ ID NO: 931 - RNA, 43 nt, synthetic construct
  source 1..43: mol_type = other RNA
gattgctccttacgaggagacatcttttctcaaataaatttta

SEQ ID NO: 932 - RNA, 43 nt, synthetic construct
  source 1..43: mol_type = other RNA
gattgctccttacgaggagacttatttgagaaaagatttttta

SEQ ID NO: 933 - RNA, 43 nt, synthetic construct
  source 1..43: mol_type = other RNA
gattgctccttacgaggagacttttgcctttttggtattttta

SEQ ID NO: 934 - RNA, 43 nt, synthetic construct
  source 1..43: mol_type = other RNA
gattgctccttacgaggagactatttctttctttgccatttta

SEQ ID NO: 935 - RNA, 43 nt, synthetic construct
  source 1..43: mol_type = other RNA
gattgctccttacgaggagacttgttttgcctttttggtttta

SEQ ID NO: 936 - RNA, 43 nt, synthetic construct
  source 1..43: mol_type = other RNA
gattgctccttacgaggagactctcaaataaaaagacatttta

SEQ ID NO: 937 - RNA, 43 nt, synthetic construct
  source 1..43: mol_type = other RNA
gattgctccttacgaggagactgttttgcctttttggttttta

SEQ ID NO: 938 - RNA, 43 nt, synthetic construct
  source 1..43: mol_type = other RNA
gattgctccttacgaggagactgtatttctttctttgctttta

SEQ ID NO: 939 - RNA, 43 nt, synthetic construct
  source 1..43: mol_type = other RNA
gattgctccttacgaggagacatttttgttttgccttttttta

SEQ ID NO: 940 - RNA, 43 nt, synthetic construct
  source 1..43: mol_type = other RNA
gattgctccttacgaggagacctattagatctgtcgcctttta

SEQ ID NO: 941 - RNA, 43 nt, synthetic construct
  source 1..43: mol_type = other RNA
gattgctccttacgaggagacgatctgtcgccctaccttttta

SEQ ID NO: 942 - RNA, 43 nt, synthetic construct
  source 1..43: mol_type = other RNA
gattgctccttacgaggagacaccctgcttataatctctttta

SEQ ID NO: 943 - RNA, 43 nt, synthetic construct
  source 1..43: mol_type = other RNA
gattgctccttacgaggagacaagaacctatcaaccattttta

SEQ ID NO: 944 - RNA, 43 nt, synthetic construct
  source 1..43: mol_type = other RNA
gattgctccttacgaggagacgtgcctttcaccctgcttttta

SEQ ID NO: 945 - RNA, 43 nt, synthetic construct
  source 1..43: mol_type = other RNA
gattgctccttacgaggagactaagcagggtgaaaggctttta

SEQ ID NO: 946 - RNA, 43 nt, synthetic construct
  source 1..43: mol_type = other RNA
gattgctccttacgaggagaccaccctgcttataatcttttta

SEQ ID NO: 947 - RNA, 43 nt, synthetic construct
  source 1..43: mol_type = other RNA
gattgctccttacgaggagaccatgagagattataagctttta

SEQ ID NO: 948 - RNA, 43 nt, synthetic construct
  source 1..43: mol_type = other RNA
gattgctccttacgaggagactagcagactttttaagctttta

SEQ ID NO: 949 - RNA, 43 nt, synthetic construct
  source 1..43: mol_type = other RNA
gattgctccttacgaggagacattaatggttgataggttttta

SEQ ID NO: 950 - RNA, 43 nt, synthetic construct
  source 1..43: mol_type = other RNA
gattgctccttacgaggagacatgttagtgcctttcactttta

SEQ ID NO: 951 - RNA, 43 nt, synthetic construct
  source 1..43: mol_type = other RNA
gattgctccttacgaggagacactgctgttgattaatgtttta

SEQ ID NO: 952 - RNA, 43 nt, synthetic construct
  source 1..43: mol_type = other RNA
gattgctccttacgaggagacctgctgttgattaatggtttta

SEQ ID NO: 953 - RNA, 43 nt, synthetic construct
  source 1..43: mol_type = other RNA
gattgctccttacgaggagactgtctgacagctgtttgtttta

SEQ ID NO: 954 - RNA, 43 nt, synthetic construct
  source 1..43: mol_type = other RNA
gattgctccttacgaggagacagcagactttttaagcttttta

SEQ ID NO: 955 - RNA, 43 nt, synthetic construct
  source 1..43: mol_type = other RNA
gattgctccttacgaggagacatcaacagcagtaaagatttta

SEQ ID NO: 956 - RNA, 43 nt, synthetic construct
  source 1..43: mol_type = other RNA
gattgctccttacgaggagaccattcctattagatctgtttta

SEQ ID NO: 957 - RNA, 43 nt, synthetic construct
  source 1..43: mol_type = other RNA
gattgctccttacgaggagacttctgtctgacagctgttttta

SEQ ID NO: 958 - RNA, 43 nt, synthetic construct
  source 1..43: mol_type = other RNA
gattgctccttacgaggagacatgagagattataagcatttta

SEQ ID NO: 959 - RNA, 43 nt, synthetic construct
  source 1..43: mol_type = other RNA
gattgctccttacgaggagactctgtctgacagctgtttttta

SEQ ID NO: 960 - RNA, 43 nt, synthetic construct
  source 1..43: mol_type = other RNA
gattgctccttacgaggagacgcagactttttaagctttttta

SEQ ID NO: 961 - RNA, 43 nt, synthetic construct
  source 1..43: mol_type = other RNA
gattgctccttacgaggagacatggttgataggttctttttta

SEQ ID NO: 962 - RNA, 43 nt, synthetic construct
  source 1..43: mol_type = other RNA
gattgctccttacgaggagacctgtctgacagctgttttttta

SEQ ID NO: 963 - RNA, 43 nt, synthetic construct
  source 1..43: mol_type = other RNA
gattgctccttacgaggagacaatgttagtgcctttcatttta

SEQ ID NO: 964 - RNA, 43 nt, synthetic construct
  source 1..43: mol_type = other RNA
gattgctccttacgaggagacattcctattagatctgttttta

SEQ ID NO: 965 - RNA, 43 nt, synthetic construct
  source 1..43: mol_type = other RNA
gattgctccttacgaggagactttctgtctgacagctgtttta

SEQ ID NO: 966 - RNA, 43 nt, synthetic construct
  source 1..43: mol_type = other RNA
gattgctccttacgaggagacgaagaatatttcatgagtttta

SEQ ID NO: 967 - RNA, 43 nt, synthetic construct
  source 1..43: mol_type = other RNA
gattgctccttacgaggagactttaatgttagtgcctttttta

SEQ ID NO: 968 - RNA, 43 nt, synthetic construct
  source 1..43: mol_type = other RNA
gattgctccttacgaggagacagaagaatatttcatgatttta

SEQ ID NO: 969 - RNA, 43 nt, synthetic construct
  source 1..43: mol_type = other RNA
gattgctccttacgaggagactcattcctattagatcttttta

SEQ ID NO: 970 - RNA, 43 nt, synthetic construct
  source 1..43: mol_type = other RNA
gattgctccttacgaggagactttactgctgttgattatttta

SEQ ID NO: 971 - RNA, 43 nt, synthetic construct
  source 1..43: mol_type = other RNA
gattgctccttacgaggagactaagctttctttagaagtttta

SEQ ID NO: 972 - RNA, 43 nt, synthetic construct
  source 1..43: mol_type = other RNA
gattgctccttacgaggagacagctttctttagaagaatttta

SEQ ID NO: 973 - RNA, 43 nt, synthetic construct
  source 1..43: mol_type = other RNA
gattgctccttacgaggagacctttactgctgttgatttttta

SEQ ID NO: 974 - RNA, 43 nt, synthetic construct
  source 1..43: mol_type = other RNA
gattgctccttacgaggagacttctaaagaaagcttaatttta

SEQ ID NO: 975 - RNA, 43 nt, synthetic construct
  source 1..43: mol_type = other RNA
gattgctccttacgaggagacataggttctttaatgtttttta

SEQ ID NO: 976 - RNA, 43 nt, synthetic construct
  source 1..43: mol_type = other RNA
gattgctccttacgaggagacaaaagtctgctaaaatgtttta

SEQ ID NO: 977 - RNA, 43 nt, synthetic construct
  source 1..43: mol_type = other RNA
gattgctccttacgaggagacaagctttctttagaagatttta

SEQ ID NO: 978 - RNA, 43 nt, synthetic construct
  source 1..43: mol_type = other RNA
gattgctccttacgaggagacttaagctttctttagaatttta

SEQ ID NO: 979 - RNA, 43 nt, synthetic construct
  source 1..43: mol_type = other RNA
gattgctccttacgaggagactaatctctcatgaaatatttta

SEQ ID NO: 980 - RNA, 43 nt, synthetic construct
  source 1..43: mol_type = other RNA
gattgctccttacgaggagacctttagaagaatatttctttta

SEQ ID NO: 981 - RNA, 43 nt, synthetic construct
  source 1..43: mol_type = other RNA
gattgctccttacgaggagactttagaagaatatttcatttta

SEQ ID NO: 982 - RNA, 43 nt, synthetic construct
  source 1..43: mol_type = other RNA
gattgctccttacgaggagacatcgaataagtaatgtgtttta

SEQ ID NO: 983 - RNA, 43 nt, synthetic construct
  source 1..43: mol_type = other RNA
gattgctccttacgaggagaccttattcgattaacacttttta

SEQ ID NO: 984 - RNA, 43 nt, synthetic construct
  source 1..43: mol_type = other RNA
gattgctccttacgaggagacttcgattaacactttgatttta

SEQ ID NO: 985 - RNA, 43 nt, synthetic construct
  source 1..43: mol_type = other RNA
gattgctccttacgaggagacgaagatctgagctctgatttta

SEQ ID NO: 986 - RNA, 43 nt, synthetic construct
  source 1..43: mol_type = other RNA
gattgctccttacgaggagactacccactaaagttaattttta

SEQ ID NO: 987 - RNA, 43 nt, synthetic construct
  source 1..43: mol_type = other RNA
gattgctccttacgaggagacaccaaatggattaagattttta

SEQ ID NO: 988 - RNA, 43 nt, synthetic construct
  source 1..43: mol_type = other RNA
gattgctccttacgaggagacaaagtgttaatcgaatatttta

SEQ ID NO: 989 - RNA, 43 nt, synthetic construct
  source 1..43: mol_type = other RNA
gattgctccttacgaggagacaaagaaattctacccactttta

SEQ ID NO: 990 - RNA, 43 nt, synthetic construct
  source 1..43: mol_type = other RNA
gattgctccttacgaggagacagtgggtagaatttctttttta

SEQ ID NO: 991 - RNA, 43 nt, synthetic construct
  source 1..43: mol_type = other RNA
gattgctccttacgaggagacgattaacactttgaagatttta

SEQ ID NO: 992 - RNA, 43 nt, synthetic construct
  source 1..43: mol_type = other RNA
gattgctccttacgaggagacatgaacatcttaatccatttta

SEQ ID NO: 993 - RNA, 43 nt, synthetic construct
  source 1..43: mol_type = other RNA
gattgctccttacgaggagacggtgaatatattattggtttta

SEQ ID NO: 994 - RNA, 43 nt, synthetic construct
  source 1..43: mol_type = other RNA
gattgctccttacgaggagacaacagaaaagcatacactttta

SEQ ID NO: 995 - RNA, 43 nt, synthetic construct
  source 1..43: mol_type = other RNA
gattgctccttacgaggagactctgttaaagaggaagttttta

SEQ ID NO: 996 - RNA, 43 nt, synthetic construct
  source 1..43: mol_type = other RNA
gattgctccttacgaggagactgttaaagaggaagttatttta

SEQ ID NO: 997 - RNA, 43 nt, synthetic construct
  source 1..43: mol_type = other RNA
gattgctccttacgaggagactcttcaaagtgttaatctttta

SEQ ID NO: 998 - RNA, 43 nt, synthetic construct
  source 1..43: mol_type = other RNA
gattgctccttacgaggagacctgttaaagaggaagtttttta

SEQ ID NO: 999 - RNA, 43 nt, synthetic construct
  source 1..43: mol_type = other RNA
gattgctccttacgaggagacgtgaatatattattggatttta

SEQ ID NO: 1000 - RNA, 43 nt, synthetic construct
  source 1..43: mol_type = other RNA
gattgctccttacgaggagacatccatttggtgaatattttta

SEQ ID NO: 1001 - RNA, 43 nt, synthetic construct
  source 1..43: mol_type = other RNA
gattgctccttacgaggagacgtgggtagaatttcttttttta

SEQ ID NO: 1002 - RNA, 43 nt, synthetic construct
  source 1..43: mol_type = other RNA
gattgctccttacgaggagacaagaggaagttagaagatttta

SEQ ID NO: 1003 - RNA, 43 nt, synthetic construct
  source 1..43: mol_type = other RNA
gattgctccttacgaggagactaacttcctctttaacatttta

SEQ ID NO: 1004 - RNA, 43 nt, synthetic construct
  source 1..43: mol_type = other RNA
gattgctccttacgaggagacaagataattcatgaacatttta

SEQ ID NO: 1005 - RNA, 43 nt, synthetic construct
  source 1..43: mol_type = other RNA
gattgctccttacgaggagacagatgttcatgaattattttta

SEQ ID NO: 1006 - RNA, 43 nt, synthetic construct
  source 1..43: mol_type = other RNA
gattgctccttacgaggagactaaattaactttagtggtttta

SEQ ID NO: 1007 - RNA, 43 nt, synthetic construct
  source 1..43: mol_type = other RNA
gattgctccttacgaggagacaaaagaaattctacccatttta

SEQ ID NO: 1008 - RNA, 43 nt, synthetic construct
  source 1..43: mol_type = other RNA
gattgctccttacgaggagacctctttaacagaaaagctttta

SEQ ID NO: 1009 - RNA, 43 nt, synthetic construct
  source 1..43: mol_type = other RNA
gattgctccttacgaggagacgaagtaaaatataatagtttta

SEQ ID NO: 1010 - RNA, 43 nt, synthetic construct
  source 1..43: mol_type = other RNA
gattgctccttacgaggagacacagaaaagcatacacatttta

SEQ ID NO: 1011 - RNA, 43 nt, synthetic construct
  source 1..43: mol_type = other RNA
gattgctccttacgaggagacatgaattatcttcaaagtttta

SEQ ID NO: 1012 - RNA, 43 nt, synthetic construct
  source 1..43: mol_type = other RNA
gattgctccttacgaggagacacactttgaagataatttttta

SEQ ID NO: 1013 - RNA, 43 nt, synthetic construct
  source 1..43: mol_type = other RNA
gattgctccttacgaggagactacttctaaattaactttttta

SEQ ID NO: 1014 - RNA, 43 nt, synthetic construct
  source 1..43: mol_type = other RNA
gattgctccttacgaggagaccttctaaattaactttatttta

SEQ ID NO: 1015 - RNA, 43 nt, synthetic construct
  source 1..43: mol_type = other RNA
gattgctccttacgaggagacacttctaaattaacttttttta

SEQ ID NO: 1016 - RNA, 43 nt, synthetic construct
  source 1..43: mol_type = other RNA
gattgctccttacgaggagacagaagtaaaatataatatttta

SEQ ID NO: 1017 - RNA, 43 nt, synthetic construct
  source 1..43: mol_type = other RNA
gattgctccttacgaggagacatttagaagtaaaatattttta

SEQ ID NO: 1018 - RNA, 43 nt, synthetic construct
  source 1..43: mol_type = other RNA
gattgctccttacgaggagacctttaaaagaaattctatttta

SEQ ID NO: 1019 - RNA, 43 nt, synthetic construct
  source 1..43: mol_type = other RNA
gattgctccttacgaggagacttggatttctattatattttta

SEQ ID NO: 1020 - RNA, 43 nt, synthetic construct
  source 1..43: mol_type = other RNA
gattgctccttacgaggagacctattatattttacttctttta

SEQ ID NO: 1021 - RNA, 43 nt, synthetic construct
  source 1..43: mol_type = other RNA
gattgctccttacgaggagacgatttctattatatttttttta

SEQ ID NO: 1022 - RNA, 43 nt, synthetic construct
  source 1..43: mol_type = other RNA
gattgctccttacgaggagactattttacttctaaatttttta

SEQ ID NO: 1023 - RNA, 43 nt, synthetic construct
  source 1..43: mol_type = other RNA
gattgctccttacgaggagactattatattttacttcttttta

SEQ ID NO: 1024 - RNA, 43 nt, synthetic construct
  source 1..43: mol_type = other RNA
gattgctccttacgaggagaccaggctccaatagtggttttta

SEQ ID NO: 1025 - RNA, 43 nt, synthetic construct
  source 1..43: mol_type = other RNA
gattgctccttacgaggagacgagcctgtaagtatacttttta

SEQ ID NO: 1026 - RNA, 43 nt, synthetic construct
  source 1..43: mol_type = other RNA
gattgctccttacgaggagacaacaaatagctagagcctttta

SEQ ID NO: 1027 - RNA, 43 nt, synthetic construct
  source 1..43: mol_type = other RNA
gattgctccttacgaggagacgtgttattgactgtgcatttta

SEQ ID NO: 1028 - RNA, 43 nt, synthetic construct
  source 1..43: mol_type = other RNA
gattgctccttacgaggagacgaacaaatagctagagctttta

SEQ ID NO: 1029 - RNA, 43 nt, synthetic construct
  source 1..43: mol_type = other RNA
gattgctccttacgaggagacggctctagctatttgtttttta

SEQ ID NO: 1030 - RNA, 43 nt, synthetic construct
  source 1..43: mol_type = other RNA
gattgctccttacgaggagacgcacagtcaataacacatttta

SEQ ID NO: 1031 - RNA, 43 nt, synthetic construct
  source 1..43: mol_type = other RNA
gattgctccttacgaggagactgcacagtcaataacactttta

SEQ ID NO: 1032 - RNA, 43 nt, synthetic construct
  source 1..43: mol_type = other RNA
gattgctccttacgaggagacgttcaaaagtgcaactatttta

SEQ ID NO: 1033 - RNA, 43 nt, synthetic construct
  source 1..43: mol_type = other RNA
gattgctccttacgaggagacgctctagctatttgttctttta

SEQ ID NO: 1034 - RNA, 43 nt, synthetic construct
  source 1..43: mol_type = other RNA
gattgctccttacgaggagacttgcacagtcaataacatttta

SEQ ID NO: 1035 - RNA, 43 nt, synthetic construct
  source 1..43: mol_type = other RNA
gattgctccttacgaggagactgaacaaatagctagagtttta

SEQ ID NO: 1036 - RNA, 43 nt, synthetic construct
  source 1..43: mol_type = other RNA
gattgctccttacgaggagactgttattgactgtgcaatttta

SEQ ID NO: 1037 - RNA, 43 nt, synthetic construct
  source 1..43: mol_type = other RNA
gattgctccttacgaggagaccacagtcaataacacaatttta

SEQ ID NO: 1038 - RNA, 43 nt, synthetic construct
  source 1..43: mol_type = other RNA
gattgctccttacgaggagactctctcacccagtcatctttta

SEQ ID NO: 1039 - RNA, 43 nt, synthetic construct
  source 1..43: mol_type = other RNA
gattgctccttacgaggagacttgactgtgcaaaaagttttta

SEQ ID NO: 1040 - RNA, 43 nt, synthetic construct
  source 1..43: mol_type = other RNA
gattgctccttacgaggagactctttggctctagctattttta

SEQ ID NO: 1041 - RNA, 43 nt, synthetic construct
  source 1..43: mol_type = other RNA
gattgctccttacgaggagactctctctcacccagtcatttta

SEQ ID NO: 1042 - RNA, 43 nt, synthetic construct
  source 1..43: mol_type = other RNA
gattgctccttacgaggagacactgtgcaaaaagtctttttta

SEQ ID NO: 1043 - RNA, 43 nt, synthetic construct
  source 1..43: mol_type = other RNA
gattgctccttacgaggagacttcaaaagtgcaactattttta

SEQ ID NO: 1044 - RNA, 43 nt, synthetic construct
  source 1..43: mol_type = other RNA
gattgctccttacgaggagacatagttgcacttttgaatttta

SEQ ID NO: 1045 - RNA, 43 nt, synthetic construct
  source 1..43: mol_type = other RNA
gattgctccttacgaggagaccaattctaaagatagagtttta

SEQ ID NO: 1046 - RNA, 43 nt, synthetic construct
  source 1..43: mol_type = other RNA
gattgctccttacgaggagacgtttcaattctaaagattttta

SEQ ID NO: 1047 - RNA, 43 nt, synthetic construct
  source 1..43: mol_type = other RNA
gattgctccttacgaggagaccacttttgaacaaatagtttta

SEQ ID NO: 1048 - RNA, 43 nt, synthetic construct
  source 1..43: mol_type = other RNA
gattgctccttacgaggagacaaaagtgcaactatgaatttta

SEQ ID NO: 1049 - RNA, 43 nt, synthetic construct
  source 1..43: mol_type = other RNA
gattgctccttacgaggagacctctctcacccagtcattttta

SEQ ID NO: 1050 - RNA, 43 nt, synthetic construct
  source 1..43: mol_type = other RNA
gattgctccttacgaggagacagaattgaaacaaattttttta

SEQ ID NO: 1051 - RNA, 43 nt, synthetic construct
  source 1..43: mol_type = other RNA
gattgctccttacgaggagacgaattgaaacaaatttttttta

SEQ ID NO: 1052 - RNA, 43 nt, synthetic construct
  source 1..43: mol_type = other RNA
gattgctccttacgaggagactctctatctttagaatttttta

SEQ ID NO: 1053 - RNA, 43 nt, synthetic construct
  source 1..43: mol_type = other RNA
gattgctccttacgaggagacaattctaaagatagagatttta

SEQ ID NO: 1054 - RNA, 43 nt, synthetic construct
  source 1..43: mol_type = other RNA
gattgctccttacgaggagactaaagatagagataaactttta

SEQ ID NO: 1055 - RNA, 43 nt, synthetic construct
  source 1..43: mol_type = other RNA
gattgctccttacgaggagacaaacaaattttctctcttttta

SEQ ID NO: 1056 - RNA, 43 nt, synthetic construct
  source 1..43: mol_type = other RNA
gattgctccttacgaggagactttcaattctaaagatatttta

SEQ ID NO: 1057 - RNA, 43 nt, synthetic construct
  source 1..43: mol_type = other RNA
gattgctccttacgaggagacgctcctactcagactgttttta

SEQ ID NO: 1058 - RNA, 43 nt, synthetic construct
  source 1..43: mol_type = other RNA
gattgctccttacgaggagacagctcctactcagactgtttta

SEQ ID NO: 1059 - RNA, 43 nt, synthetic construct
  source 1..43: mol_type = other RNA
gattgctccttacgaggagacggctcaaattgttactctttta

SEQ ID NO: 1060 - RNA, 43 nt, synthetic construct
  source 1..43: mol_type = other RNA
gattgctccttacgaggagactagctcctactcagacttttta

SEQ ID NO: 1061 - RNA, 43 nt, synthetic construct
  source 1..43: mol_type = other RNA
gattgctccttacgaggagacgagccaaactcttattctttta

SEQ ID NO: 1062 - RNA, 43 nt, synthetic construct
  source 1..43: mol_type = other RNA
gattgctccttacgaggagacgggtttttgcaaaaaggtttta

SEQ ID NO: 1063 - RNA, 43 nt, synthetic construct
  source 1..43: mol_type = other RNA
gattgctccttacgaggagacgctcaaattgttactcttttta

SEQ ID NO: 1064 - RNA, 43 nt, synthetic construct
  source 1..43: mol_type = other RNA
gattgctccttacgaggagacaaattggctctttagcttttta

SEQ ID NO: 1065 - RNA, 43 nt, synthetic construct
  source 1..43: mol_type = other RNA
gattgctccttacgaggagacgctctttagcttgtgtttttta

SEQ ID NO: 1066 - RNA, 43 nt, synthetic construct
  source 1..43: mol_type = other RNA
gattgctccttacgaggagacagccaaactcttattcatttta

SEQ ID NO: 1067 - RNA, 43 nt, synthetic construct
  source 1..43: mol_type = other RNA
gattgctccttacgaggagaccctttttgcaaaaaccctttta

SEQ ID NO: 1068 - RNA, 43 nt, synthetic construct
  source 1..43: mol_type = other RNA
gattgctccttacgaggagacaagagtaacaatttgagtttta

SEQ ID NO: 1069 - RNA, 43 nt, synthetic construct
  source 1..43: mol_type = other RNA
gattgctccttacgaggagacgaaacacaagctaaagatttta

SEQ ID NO: 1070 - RNA, 43 nt, synthetic construct
  source 1..43: mol_type = other RNA
gattgctccttacgaggagacgacttattgttattgaatttta

SEQ ID NO: 1071 - RNA, 43 nt, synthetic construct
  source 1..43: mol_type = other RNA
gattgctccttacgaggagacgcaaaaacccaaaatattttta

SEQ ID NO: 1072 - RNA, 43 nt, synthetic construct
  source 1..43: mol_type = other RNA
gattgctccttacgaggagacggtttttgcaaaaaggatttta

SEQ ID NO: 1073 - RNA, 43 nt, synthetic construct
  source 1..43: mol_type = other RNA
gattgctccttacgaggagactcctttttgcaaaaacctttta

SEQ ID NO: 1074 - RNA, 43 nt, synthetic construct
  source 1..43: mol_type = other RNA
gattgctccttacgaggagaccaataacaataagtcaatttta

SEQ ID NO: 1075 - RNA, 43 nt, synthetic construct
  source 1..43: mol_type = other RNA
gattgctccttacgaggagacattgaagagtaacaatttttta

SEQ ID NO: 1076 - RNA, 43 nt, synthetic construct
  source 1..43: mol_type = other RNA
gattgctccttacgaggagacttgaaattggctctttatttta

SEQ ID NO: 1077 - RNA, 43 nt, synthetic construct
  source 1..43: mol_type = other RNA
gattgctccttacgaggagactgcaaaaacccaaaatatttta

SEQ ID NO: 1078 - RNA, 43 nt, synthetic construct
  source 1..43: mol_type = other RNA
gattgctccttacgaggagacttattgaaattggctcttttta

SEQ ID NO: 1079 - RNA, 43 nt, synthetic construct
  source 1..43: mol_type = other RNA
gattgctccttacgaggagacagcttgtgtttctaatttttta

SEQ ID NO: 1080 - RNA, 43 nt, synthetic construct
  source 1..43: mol_type = other RNA
gattgctccttacgaggagacttgcaaaaacccaaaattttta

SEQ ID NO: 1081 - RNA, 43 nt, synthetic construct
  source 1..43: mol_type = other RNA
gattgctccttacgaggagacttgttattgaaattggctttta

SEQ ID NO: 1082 - RNA, 43 nt, synthetic construct
  source 1..43: mol_type = other RNA
gattgctccttacgaggagacgcttgtgtttctaattttttta

SEQ ID NO: 1083 - RNA, 43 nt, synthetic construct
  source 1..43: mol_type = other RNA
gattgctccttacgaggagacttcatgacattatatattttta

SEQ ID NO: 1084 - RNA, 43 nt, synthetic construct
  source 1..43: mol_type = other RNA
gattgctccttacgaggagacctcttcaattaaatttgtttta

SEQ ID NO: 1085 - RNA, 43 nt, synthetic construct
  source 1..43: mol_type = other RNA
gattgctccttacgaggagacttactcttcaattaaattttta

SEQ ID NO: 1086 - RNA, 43 nt, synthetic construct
  source 1..43: mol_type = other RNA
gattgctccttacgaggagaccaaaaacccaaaatatttttta

SEQ ID NO: 1087 - RNA, 43 nt, synthetic construct
  source 1..43: mol_type = other RNA
gattgctccttacgaggagacttcctttttgcaaaaactttta

SEQ ID NO: 1088 - RNA, 43 nt, synthetic construct
  source 1..43: mol_type = other RNA
gattgctccttacgaggagacctttttgcaaaaacccatttta

SEQ ID NO: 1089 - RNA, 43 nt, synthetic construct
  source 1..43: mol_type = other RNA
gattgctccttacgaggagacacttattgttattgaaatttta

SEQ ID NO: 1090 - RNA, 43 nt, synthetic construct
  source 1..43: mol_type = other RNA
gattgctccttacgaggagacaatttgacttattgttatttta

SEQ ID NO: 1091 - RNA, 43 nt, synthetic construct
  source 1..43: mol_type = other RNA
gattgctccttacgaggagacaattaaatttgacttattttta

SEQ ID NO: 1092 - RNA, 43 nt, synthetic construct
  source 1..43: mol_type = other RNA
gattgctccttacgaggagactttcctttttgcaaaaatttta

SEQ ID NO: 1093 - RNA, 43 nt, synthetic construct
  source 1..43: mol_type = other RNA
gattgctccttacgaggagacgcaaaaaggaaaaaagatttta

SEQ ID NO: 1094 - RNA, 43 nt, synthetic construct
  source 1..43: mol_type = other RNA
gattgctccttacgaggagactctttttcttctttttttttta

SEQ ID NO: 1095 - RNA, 43 nt, synthetic construct
  source 1..43: mol_type = other RNA
gattgctccttacgaggagaccaaaaaggaaaaaagaatttta

SEQ ID NO: 1096 - RNA, 43 nt, synthetic construct
  source 1..43: mol_type = other RNA
gattgctccttacgaggagacttttttcctttttgcaatttta

SEQ ID NO: 1097 - RNA, 43 nt, synthetic construct
  source 1..43: mol_type = other RNA
gattgctccttacgaggagacttcttttttcctttttgtttta

SEQ ID NO: 1098 - RNA, 43 nt, synthetic construct
  source 1..43: mol_type = other RNA
gattgctccttacgaggagactgtttctaatttttctttttta

SEQ ID NO: 1099 - RNA, 43 nt, synthetic construct
  source 1..43: mol_type = other RNA
gattgctccttacgaggagacctaatttttctttttcttttta

SEQ ID NO: 1100 - RNA, 43 nt, synthetic construct
  source 1..43: mol_type = other RNA
gattgctccttacgaggagactttttcttcttttttcctttta

SEQ ID NO: 1101 - RNA, 43 nt, synthetic construct
  source 1..43: mol_type = other RNA
gattgctccttacgaggagactcttcttttttcctttttttta

SEQ ID NO: 1102 - RNA, 43 nt, synthetic construct
  source 1..43: mol_type = other RNA
gattgctccttacgaggagactaatttttctttttctttttta

SEQ ID NO: 1103 - RNA, 43 nt, synthetic construct
  source 1..43: mol_type = other RNA
gattgctccttacgaggagacttcttcttttttccttttttta

SEQ ID NO: 1104 - RNA, 43 nt, synthetic construct
  source 1..43: mol_type = other RNA
gattgctccttacgaggagaccttcttttttccttttttttta

SEQ ID NO: 1105 - RNA, 43 nt, synthetic construct
  source 1..43: mol_type = other RNA
gattgctccttacgaggagacctttttcttcttttttctttta

SEQ ID NO: 1106 - RNA, 43 nt, synthetic construct
  source 1..43: mol_type = other RNA
gattgctccttacgaggagacttctttttcttcttttttttta

SEQ ID NO: 1107 - RNA, 43 nt, synthetic construct
  source 1..43: mol_type = other RNA
gattgctccttacgaggagaccccaccagttcttaggctttta

SEQ ID NO: 1108 - RNA, 43 nt, synthetic construct
  source 1..43: mol_type = other RNA
gattgctccttacgaggagaccctaagaactggtgggatttta

SEQ ID NO: 1109 - RNA, 43 nt, synthetic construct
  source 1..43: mol_type = other RNA
gattgctccttacgaggagacccaccagttcttaggcatttta

SEQ ID NO: 1110 - RNA, 43 nt, synthetic construct
  source 1..43: mol_type = other RNA
gattgctccttacgaggagacatgatcatctcgttgattttta

SEQ ID NO: 1111 - RNA, 43 nt, synthetic construct
  source 1..43: mol_type = other RNA
gattgctccttacgaggagactgcttgatgatcatctctttta

SEQ ID NO: 1112 - RNA, 43 nt, synthetic construct
  source 1..43: mol_type = other RNA
gattgctccttacgaggagaccaccaatcactttactctttta

SEQ ID NO: 1113 - RNA, 43 nt, synthetic construct
  source 1..43: mol_type = other RNA
gattgctccttacgaggagactccaccaatcactttactttta

SEQ ID NO: 1114 - RNA, 43 nt, synthetic construct
  source 1..43: mol_type = other RNA
gattgctccttacgaggagacactctcctagaccattttttta

SEQ ID NO: 1115 - RNA, 43 nt, synthetic construct
  source 1..43: mol_type = other RNA
gattgctccttacgaggagacccaccaatcactttacttttta

SEQ ID NO: 1116 - RNA, 43 nt, synthetic construct
  source 1..43: mol_type = other RNA
gattgctccttacgaggagacctctcctagaccatttctttta

SEQ ID NO: 1117 - RNA, 43 nt, synthetic construct
  source 1..43: mol_type = other RNA
gattgctccttacgaggagacgctgagagagaaacagttttta

SEQ ID NO: 1118 - RNA, 43 nt, synthetic construct
  source 1..43: mol_type = other RNA
gattgctccttacgaggagacctctcactattcagccttttta

SEQ ID NO: 1119 - RNA, 43 nt, synthetic construct
  source 1..43: mol_type = other RNA
gattgctccttacgaggagactcataccttctgcttgatttta

SEQ ID NO: 1120 - RNA, 43 nt, synthetic construct
  source 1..43: mol_type = other RNA
gattgctccttacgaggagacttaggcaactgtttctctttta

SEQ ID NO: 1121 - RNA, 43 nt, synthetic construct
  source 1..43: mol_type = other RNA
gattgctccttacgaggagacctcataccttctgcttgtttta

SEQ ID NO: 1122 - RNA, 43 nt, synthetic construct
  source 1..43: mol_type = other RNA
gattgctccttacgaggagacggcaactgtttctctcttttta

SEQ ID NO: 1123 - RNA, 43 nt, synthetic construct
  source 1..43: mol_type = other RNA
gattgctccttacgaggagacctctctcagcaaacacatttta

SEQ ID NO: 1124 - RNA, 43 nt, synthetic construct
  source 1..43: mol_type = other RNA
gattgctccttacgaggagacaaagaaaaacttctgcctttta

SEQ ID NO: 1125 - RNA, 43 nt, synthetic construct
  source 1..43: mol_type = other RNA
gattgctccttacgaggagactctctcagcaaacacattttta

SEQ ID NO: 1126 - RNA, 43 nt, synthetic construct
  source 1..43: mol_type = other RNA
gattgctccttacgaggagacaagaaaaacttctgccatttta

SEQ ID NO: 1127 - RNA, 43 nt, synthetic construct
  source 1..43: mol_type = other RNA
gattgctccttacgaggagacttctcataccttctgcttttta

SEQ ID NO: 1128 - RNA, 43 nt, synthetic construct
  source 1..43: mol_type = other RNA
gattgctccttacgaggagacctgagagagaaacagtttttta

SEQ ID NO: 1129 - RNA, 43 nt, synthetic construct
  source 1..43: mol_type = other RNA
gattgctccttacgaggagacgcagaagtttttctttatttta

SEQ ID NO: 1130 - RNA, 43 nt, synthetic construct
  source 1..43: mol_type = other RNA
gattgctccttacgaggagactaaagaaaaacttctgctttta

SEQ ID NO: 1131 - RNA, 43 nt, synthetic construct
  source 1..43: mol_type = other RNA
gattgctccttacgaggagacgtggaaaatcttcattttttta

SEQ ID NO: 1132 - RNA, 43 nt, synthetic construct
  source 1..43: mol_type = other RNA
gattgctccttacgaggagactttctcataccttctgctttta

SEQ ID NO: 1133 - RNA, 43 nt, synthetic construct
  source 1..43: mol_type = other RNA
gattgctccttacgaggagacaaatgaagattttccactttta

SEQ ID NO: 1134 - RNA, 43 nt, synthetic construct
  source 1..43: mol_type = other RNA
gattgctccttacgaggagactgccaacttttatcatttttta

SEQ ID NO: 1135 - RNA, 43 nt, synthetic construct
  source 1..43: mol_type = other RNA
gattgctccttacgaggagactctcataccttctgctttttta

SEQ ID NO: 1136 - RNA, 43 nt, synthetic construct
  source 1..43: mol_type = other RNA
gattgctccttacgaggagacaaaatgaagattttccatttta

SEQ ID NO: 1137 - RNA, 43 nt, synthetic construct
  source 1..43: mol_type = other RNA
gattgctccttacgaggagactcattttttctcatacctttta

SEQ ID NO: 1138 - RNA, 43 nt, synthetic construct
  source 1..43: mol_type = other RNA
gattgctccttacgaggagactatcattttttctcatatttta

SEQ ID NO: 1139 - RNA, 43 nt, synthetic construct
  source 1..43: mol_type = other RNA
gattgctccttacgaggagacatcattttttctcatactttta

SEQ ID NO: 1140 - RNA, 43 nt, synthetic construct
  source 1..43: mol_type = other RNA
gattgctccttacgaggagacctttaaaatgaagattttttta

SEQ ID NO: 1141 - RNA, 43 nt, synthetic construct
  source 1..43: mol_type = other RNA
gattgctccttacgaggagactctttaaaatgaagatttttta

SEQ ID NO: 1142 - RNA, 43 nt, synthetic construct
  source 1..43: mol_type = other RNA
gattgctccttacgaggagacttctttaaaatgaagattttta

SEQ ID NO: 1143 - RNA, 43 nt, synthetic construct
  source 1..43: mol_type = other RNA
gattgctccttacgaggagacattttaaagaaaaactttttta

SEQ ID NO: 1144 - RNA, 43 nt, synthetic construct
  source 1..43: mol_type = other RNA
gattgctccttacgaggagactttaaaatgaagatttttttta

SEQ ID NO: 1145 - RNA, 43 nt, synthetic construct
  source 1..43: mol_type = other RNA
gattgctccttacgaggagacatcatcctagccataactttta

SEQ ID NO: 1146 - RNA, 43 nt, synthetic construct
  source 1..43: mol_type = other RNA
gattgctccttacgaggagactgttatggctaggatgatttta

SEQ ID NO: 1147 - RNA, 43 nt, synthetic construct
  source 1..43: mol_type = other RNA
gattgctccttacgaggagacaatgtaacttccaaacgtttta

SEQ ID NO: 1148 - RNA, 43 nt, synthetic construct
  source 1..43: mol_type = other RNA
gattgctccttacgaggagacattgtgttatggctaggtttta

SEQ ID NO: 1149 - RNA, 43 nt, synthetic construct
  source 1..43: mol_type = other RNA
gattgctccttacgaggagacttcatcatcctagccattttta

SEQ ID NO: 1150 - RNA, 43 nt, synthetic construct
  source 1..43: mol_type = other RNA
gattgctccttacgaggagacatcccactgattctgaatttta

SEQ ID NO: 1151 - RNA, 43 nt, synthetic construct
  source 1..43: mol_type = other RNA
gattgctccttacgaggagacaaatgtctcctccagactttta

SEQ ID NO: 1152 - RNA, 43 nt, synthetic construct
  source 1..43: mol_type = other RNA
gattgctccttacgaggagacttcattgtgttatggcttttta

SEQ ID NO: 1153 - RNA, 43 nt, synthetic construct
  source 1..43: mol_type = other RNA
gattgctccttacgaggagacagaatcagtgggatgaatttta

SEQ ID NO: 1154 - RNA, 43 nt, synthetic construct
  source 1..43: mol_type = other RNA
gattgctccttacgaggagacaaaatgtctcctccagatttta

SEQ ID NO: 1155 - RNA, 43 nt, synthetic construct
  source 1..43: mol_type = other RNA
gattgctccttacgaggagaccaaacgttatctcacattttta

SEQ ID NO: 1156 - RNA, 43 nt, synthetic construct
  source 1..43: mol_type = other RNA
gattgctccttacgaggagacggaagttacatttaaaatttta

SEQ ID NO: 1157 - RNA, 43 nt, synthetic construct
  source 1..43: mol_type = other RNA
gattgctccttacgaggagactgaattctttcaactagtttta

SEQ ID NO: 1158 - RNA, 43 nt, synthetic construct
  source 1..43: mol_type = other RNA
gattgctccttacgaggagaccaactagaataaaaggatttta

SEQ ID NO: 1159 - RNA, 43 nt, synthetic construct
  source 1..43: mol_type = other RNA
gattgctccttacgaggagactctcacatttatgttgctttta

SEQ ID NO: 1160 - RNA, 43 nt, synthetic construct
  source 1..43: mol_type = other RNA
gattgctccttacgaggagacattctagttgaaagaattttta

SEQ ID NO: 1161 - RNA, 43 nt, synthetic construct
  source 1..43: mol_type = other RNA
gattgctccttacgaggagaccatttaaaatgtctccttttta

SEQ ID NO: 1162 - RNA, 43 nt, synthetic construct
  source 1..43: mol_type = other RNA
gattgctccttacgaggagactagttgaaagaattcagtttta

SEQ ID NO: 1163 - RNA, 43 nt, synthetic construct
  source 1..43: mol_type = other RNA
gattgctccttacgaggagacaaagaattcagaatcagtttta

SEQ ID NO: 1164 - RNA, 43 nt, synthetic construct
  source 1..43: mol_type = other RNA
gattgctccttacgaggagacaataagcaacataaatgtttta

SEQ ID NO: 1165 - RNA, 43 nt, synthetic construct
  source 1..43: mol_type = other RNA
gattgctccttacgaggagacaactagaataaaaggaatttta

SEQ ID NO: 1166 - RNA, 43 nt, synthetic construct
  source 1..43: mol_type = other RNA
gattgctccttacgaggagacccttttattctagttgatttta

SEQ ID NO: 1167 - RNA, 43 nt, synthetic construct
  source 1..43: mol_type = other RNA
gattgctccttacgaggagacttctagttgaaagaatttttta

SEQ ID NO: 1168 - RNA, 43 nt, synthetic construct
  source 1..43: mol_type = other RNA
gattgctccttacgaggagactattctagttgaaagaatttta

SEQ ID NO: 1169 - RNA, 43 nt, synthetic construct
  source 1..43: mol_type = other RNA
gattgctccttacgaggagacttaaataagcaacataatttta

SEQ ID NO: 1170 - RNA, 43 nt, synthetic construct
  source 1..43: mol_type = other RNA
gattgctccttacgaggagactaaataagcaacataaatttta

SEQ ID NO: 1171 - RNA, 43 nt, synthetic construct
  source 1..43: mol_type = other RNA
gattgctccttacgaggagactttaaataagcaacatatttta

SEQ ID NO: 1172 - RNA, 43 nt, synthetic construct
  source 1..43: mol_type = other RNA
gattgctccttacgaggagacaaaaattattcattgtgtttta

SEQ ID NO: 1173 - RNA, 43 nt, synthetic construct
  source 1..43: mol_type = other RNA
gattgctccttacgaggagacaaataagcaacataaattttta

SEQ ID NO: 1174 - RNA, 43 nt, synthetic construct
  source 1..43: mol_type = other RNA
gattgctccttacgaggagactccttttattctagttgtttta

SEQ ID NO: 1175 - RNA, 43 nt, synthetic construct
  source 1..43: mol_type = other RNA
gattgctccttacgaggagacgaagttacatttaaaattttta

SEQ ID NO: 1176 - RNA, 43 nt, synthetic construct
  source 1..43: mol_type = other RNA
gattgctccttacgaggagaccttttattctagttgaatttta

SEQ ID NO: 1177 - RNA, 43 nt, synthetic construct
  source 1..43: mol_type = other RNA
gattgctccttacgaggagacactactatatatttatttttta

SEQ ID NO: 1178 - RNA, 43 nt, synthetic construct
  source 1..43: mol_type = other RNA
gattgctccttacgaggagacaaaaaattattcattgttttta

SEQ ID NO: 1179 - RNA, 43 nt, synthetic construct
  source 1..43: mol_type = other RNA
gattgctccttacgaggagacttccttttattctagtttttta

SEQ ID NO: 1180 - RNA, 43 nt, synthetic construct
  source 1..43: mol_type = other RNA
gattgctccttacgaggagacctactatatatttattttttta

SEQ ID NO: 1181 - RNA, 43 nt, synthetic construct
  source 1..43: mol_type = other RNA
gattgctccttacgaggagactttttccttttattctatttta

SEQ ID NO: 1182 - RNA, 43 nt, synthetic construct
  source 1..43: mol_type = other RNA
gattgctccttacgaggagacatttttccttttattcttttta

SEQ ID NO: 1183 - RNA, 43 nt, synthetic construct
  source 1..43: mol_type = other RNA
gattgctccttacgaggagaccagctttaacgtgattttttta

SEQ ID NO: 1184 - RNA, 43 nt, synthetic construct
  source 1..43: mol_type = other RNA
gattgctccttacgaggagacgatactaaccttggttttttta

SEQ ID NO: 1185 - RNA, 43 nt, synthetic construct
  source 1..43: mol_type = other RNA
gattgctccttacgaggagacatttcagctttaacgtgtttta

SEQ ID NO: 1186 - RNA, 43 nt, synthetic construct
  source 1..43: mol_type = other RNA
gattgctccttacgaggagacatactaaccttggtttctttta

SEQ ID NO: 1187 - RNA, 43 nt, synthetic construct
  source 1..43: mol_type = other RNA
gattgctccttacgaggagacgtatcaaagataccttttttta

SEQ ID NO: 1188 - RNA, 43 nt, synthetic construct
  source 1..43: mol_type = other RNA
gattgctccttacgaggagacacagaaaatcacgttaatttta

SEQ ID NO: 1189 - RNA, 43 nt, synthetic construct
  source 1..43: mol_type = other RNA
gattgctccttacgaggagacttaacagaaaatcacgttttta

SEQ ID NO: 1190 - RNA, 43 nt, synthetic construct
  source 1..43: mol_type = other RNA
gattgctccttacgaggagactatcaaatgtaaccagttttta

SEQ ID NO: 1191 - RNA, 43 nt, synthetic construct
  source 1..43: mol_type = other RNA
gattgctccttacgaggagacaattacaatctatggtatttta

SEQ ID NO: 1192 - RNA, 43 nt, synthetic construct
  source 1..43: mol_type = other RNA
gattgctccttacgaggagacatcaaatgtaaccagtatttta

SEQ ID NO: 1193 - RNA, 43 nt, synthetic construct
  source 1..43: mol_type = other RNA
gattgctccttacgaggagacagctttaacgtgatttttttta

SEQ ID NO: 1194 - RNA, 43 nt, synthetic construct
  source 1..43: mol_type = other RNA
gattgctccttacgaggagacacgtgattttctgttaatttta

SEQ ID NO: 1195 - RNA, 43 nt, synthetic construct
  source 1..43: mol_type = other RNA
gattgctccttacgaggagacaacgtgattttctgttatttta

SEQ ID NO: 1196 - RNA, 43 nt, synthetic construct
  source 1..43: mol_type = other RNA
gattgctccttacgaggagactcaaatgtaaccagtattttta

SEQ ID NO: 1197 - RNA, 43 nt, synthetic construct
  source 1..43: mol_type = other RNA
gattgctccttacgaggagactaccatagattgtaatttttta

SEQ ID NO: 1198 - RNA, 43 nt, synthetic construct
  source 1..43: mol_type = other RNA
gattgctccttacgaggagaccaatctatggtataatttttta

SEQ ID NO: 1199 - RNA, 43 nt, synthetic construct
  source 1..43: mol_type = other RNA
gattgctccttacgaggagacaagctgaaatgaacagttttta

SEQ ID NO: 1200 - RNA, 43 nt, synthetic construct
  source 1..43: mol_type = other RNA
gattgctccttacgaggagacgataaaattataccatatttta

SEQ ID NO: 1201 - RNA, 43 nt, synthetic construct
  source 1..43: mol_type = other RNA
gattgctccttacgaggagacctgttaataactttacatttta

SEQ ID NO: 1202 - RNA, 43 nt, synthetic construct
  source 1..43: mol_type = other RNA
gattgctccttacgaggagactctgttaataactttactttta

SEQ ID NO: 1203 - RNA, 43 nt, synthetic construct
  source 1..43: mol_type = other RNA
gattgctccttacgaggagacaaataaaatactggttatttta

SEQ ID NO: 1204 - RNA, 43 nt, synthetic construct
  source 1..43: mol_type = other RNA
gattgctccttacgaggagacaatgatgtttaatgtaatttta

SEQ ID NO: 1205 - RNA, 43 nt, synthetic construct
  source 1..43: mol_type = other RNA
gattgctccttacgaggagacaaaaggtatctttgatatttta

SEQ ID NO: 1206 - RNA, 43 nt, synthetic construct
  source 1..43: mol_type = other RNA
gattgctccttacgaggagacacattaaacatcattaatttta

SEQ ID NO: 1207 - RNA, 43 nt, synthetic construct
  source 1..43: mol_type = other RNA
gattgctccttacgaggagacatgtaaagttattaacatttta

SEQ ID NO: 1208 - RNA, 43 nt, synthetic construct
  source 1..43: mol_type = other RNA
gattgctccttacgaggagacataaaattataccatagtttta

SEQ ID NO: 1209 - RNA, 43 nt, synthetic construct
  source 1..43: mol_type = other RNA
gattgctccttacgaggagactaaaaaggtatctttgatttta

SEQ ID NO: 1210 - RNA, 43 nt, synthetic construct
  source 1..43: mol_type = other RNA
gattgctccttacgaggagacaaaaaggtatctttgattttta

SEQ ID NO: 1211 - RNA, 43 nt, synthetic construct
  source 1..43: mol_type = other RNA
gattgctccttacgaggagacaatgtaaagttattaactttta

SEQ ID NO: 1212 - RNA, 43 nt, synthetic construct
  source 1..43: mol_type = other RNA
gattgctccttacgaggagacaacatcattaaattacatttta

SEQ ID NO: 1213 - RNA, 43 nt, synthetic construct
  source 1..43: mol_type = other RNA
gattgctccttacgaggagacatgatgtttaatgtaaatttta

SEQ ID NO: 1214 - RNA, 43 nt, synthetic construct
  source 1..43: mol_type = other RNA
gattgctccttacgaggagacaaaataaaatactggtttttta

SEQ ID NO: 1215 - RNA, 43 nt, synthetic construct
  source 1..43: mol_type = other RNA
gattgctccttacgaggagacttaaaataaaatactggtttta

SEQ ID NO: 1216 - RNA, 43 nt, synthetic construct
  source 1..43: mol_type = other RNA
gattgctccttacgaggagactaaaataaaatactggttttta

SEQ ID NO: 1217 - RNA, 43 nt, synthetic construct
  source 1..43: mol_type = other RNA
gattgctccttacgaggagacctgtgattttcttttggtttta

SEQ ID NO: 1218 - RNA, 43 nt, synthetic construct
  source 1..43: mol_type = other RNA
gattgctccttacgaggagactgttaataactttacattttta

SEQ ID NO: 1219 - RNA, 43 nt, synthetic construct
  source 1..43: mol_type = other RNA
gattgctccttacgaggagactattttaaaaaggtatctttta

SEQ ID NO: 1220 - RNA, 43 nt, synthetic construct
  source 1..43: mol_type = other RNA
gattgctccttacgaggagactaatttaatgatgtttatttta

SEQ ID NO: 1221 - RNA, 43 nt, synthetic construct
  source 1..43: mol_type = other RNA
gattgctccttacgaggagacgtttctgtgattttctttttta

SEQ ID NO: 1222 - RNA, 43 nt, synthetic construct
  source 1..43: mol_type = other RNA
gattgctccttacgaggagacattttaaaaaggtatcttttta

SEQ ID NO: 1223 - RNA, 43 nt, synthetic construct
  source 1..43: mol_type = other RNA
gattgctccttacgaggagacttttaaaaaggtatctttttta

SEQ ID NO: 1224 - RNA, 43 nt, synthetic construct
  source 1..43: mol_type = other RNA
gattgctccttacgaggagaccatttgataaaattatatttta

SEQ ID NO: 1225 - RNA, 43 nt, synthetic construct
  source 1..43: mol_type = other RNA
gattgctccttacgaggagactgtgattttcttttggatttta

SEQ ID NO: 1226 - RNA, 37 nt, synthetic construct
  source 1..37: mol_type = other RNA
attgctccttacgaggagaccatctactgtataggga

SEQ ID NO: 1227 - RNA, 37 nt, synthetic construct
  source 1..37: mol_type = other RNA
attgctccttacgaggagacgctctggcctgtcctaa

SEQ ID NO: 1228 - RNA, 37 nt, synthetic construct
  source 1..37: mol_type = other RNA
attgctccttacgaggagaccattcaactgttgcctc

SEQ ID NO: 1229 - RNA, 37 nt, synthetic construct
  source 1..37: mol_type = other RNA
attgctccttacgaggagactgaaggtgttcttgtac

SEQ ID NO: 1230 - RNA, 37 nt, synthetic construct
  source 1..37: mol_type = other RNA
attgctccttacgaggagaccttttggattgcatcta

SEQ ID NO: 1231 - RNA, 37 nt, synthetic construct
  source 1..37: mol_type = other RNA
attgctccttacgaggagaccttagcttccagccatt

SEQ ID NO: 1232 - RNA, 37 nt, synthetic construct
  source 1..37: mol_type = other RNA
attgctccttacgaggagacttttggattgcatctac

SEQ ID NO: 1233 - RNA, 37 nt, synthetic construct
  source 1..37: mol_type = other RNA
attgctccttacgaggagacggattgcatctactgta

SEQ ID NO: 1234 - RNA, 37 nt, synthetic construct
  source 1..37: mol_type = other RNA
attgctccttacgaggagacaacatttcattcaactg

SEQ ID NO: 1235 - RNA, 37 nt, synthetic construct
  source 1..37: mol_type = other RNA
attgctccttacgaggagacttgtacttcatcccact

SEQ ID NO: 1236 - RNA, 37 nt, synthetic construct
  source 1..37: mol_type = other RNA
attgctccttacgaggagactcttttggattgcatct

SEQ ID NO: 1237 - RNA, 37 nt, synthetic construct
  source 1..37: mol_type = other RNA
attgctccttacgaggagacacatttcattcaactgt

SEQ ID NO: 1238 - RNA, 37 nt, synthetic construct
  source 1..37: mol_type = other RNA
attgctccttacgaggagacttccttagcttccagcc

SEQ ID NO: 1239 - RNA, 37 nt, synthetic construct
  source 1..37: mol_type = other RNA
attgctccttacgaggagacgattgcatctactgtat

SEQ ID NO: 1240 - RNA, 37 nt, synthetic construct
  source 1..37: mol_type = other RNA
attgctccttacgaggagaccatgactcaagcttggc

SEQ ID NO: 1241 - RNA, 37 nt, synthetic construct
  source 1..37: mol_type = other RNA
attgctccttacgaggagacattcaactgttgcctcc

SEQ ID NO: 1242 - RNA, 37 nt, synthetic construct
  source 1..37: mol_type = other RNA
attgctccttacgaggagactgttgaatcctttaaca

SEQ ID NO: 1243 - RNA, 37 nt, synthetic construct
  source 1..37: mol_type = other RNA
attgctccttacgaggagactggattgcatctactgt

SEQ ID NO: 1244 - RNA, 37 nt, synthetic construct
  source 1..37: mol_type = other RNA
attgctccttacgaggagacgcttccagccattgtgt

SEQ ID NO: 1245 - RNA, 37 nt, synthetic construct
  source 1..37: mol_type = other RNA
attgctccttacgaggagaccagccattgtgttgaat

SEQ ID NO: 1246 - RNA, 37 nt, synthetic construct
  source 1..37: mol_type = other RNA
attgctccttacgaggagaccctccggttctgaaggt

SEQ ID NO: 1247 - RNA, 37 nt, synthetic construct
  source 1..37: mol_type = other RNA
attgctccttacgaggagactacttcatcccactgat

SEQ ID NO: 1248 - RNA, 37 nt, synthetic construct
  source 1..37: mol_type = other RNA
attgctccttacgaggagacaactgttgcctccggtt

SEQ ID NO: 1249 - RNA, 37 nt, synthetic construct
  source 1..37: mol_type = other RNA
attgctccttacgaggagacaaggattcaacacaatg

SEQ ID NO: 1250 - RNA, 37 nt, synthetic construct
  source 1..37: mol_type = other RNA
attgctccttacgaggagacaacacaatggctggaag

SEQ ID NO: 1251 - RNA, 37 nt, synthetic construct
  source 1..37: mol_type = other RNA
attgctccttacgaggagacggacaggccagagccaa

SEQ ID NO: 1252 - RNA, 37 nt, synthetic construct
  source 1..37: mol_type = other RNA
attgctccttacgaggagacagtcatggaaggagggt

SEQ ID NO: 1253 - RNA, 37 nt, synthetic construct
  source 1..37: mol_type = other RNA
attgctccttacgaggagacagaaccggaggcaacag

SEQ ID NO: 1254 - RNA, 37 nt, synthetic construct
  source 1..37: mol_type = other RNA
attgctccttacgaggagactataacttgatcaagca

SEQ ID NO: 1255 - RNA, 37 nt, synthetic construct
  source 1..37: mol_type = other RNA
attgctccttacgaggagacatcaagcagagaaagcc

SEQ ID NO: 1256 - RNA, 37 nt, synthetic construct
  source 1..37: mol_type = other RNA
attgctccttacgaggagactagtttggagatggcag

SEQ ID NO: 1257 - RNA, 37 nt, synthetic construct
  source 1..37: mol_type = other RNA
attgctccttacgaggagacgagatggcagtttcctt

SEQ ID NO: 1258 - RNA, 37 nt, synthetic construct
  source 1..37: mol_type = other RNA
attgctccttacgaggagacccttagtaaccacaggt

SEQ ID NO: 1259 - RNA, 37 nt, synthetic construct
  source 1..37: mol_type = other RNA
attgctccttacgaggagacatatcctcaaggtcacc

SEQ ID NO: 1260 - RNA, 37 nt, synthetic construct
  source 1..37: mol_type = other RNA
attgctccttacgaggagaccttagtaaccacaggtt

SEQ ID NO: 1261 - RNA, 37 nt, synthetic construct
  source 1..37: mol_type = other RNA
attgctccttacgaggagactgtccaagcccggttga

SEQ ID NO: 1262 - RNA, 37 nt, synthetic construct
  source 1..37: mol_type = other RNA
attgctccttacgaggagacctagtttggagatggca

SEQ ID NO: 1263 - RNA, 37 nt, synthetic construct
  source 1..37: mol_type = other RNA
attgctccttacgaggagacaaatctgccagagcagg

SEQ ID NO: 1264 - RNA, 37 nt, synthetic construct
  source 1..37: mol_type = other RNA
attgctccttacgaggagactaacttgatcaagcaga

SEQ ID NO: 1265 - RNA, 37 nt, synthetic construct
  source 1..37: mol_type = other RNA
attgctccttacgaggagacgtaaccacaggttgtgt

SEQ ID NO: 1266 - RNA, 37 nt, synthetic construct
  source 1..37: mol_type = other RNA
attgctccttacgaggagactgtcaccagagtaacag

SEQ ID NO: 1267 - RNA, 37 nt, synthetic construct
  source 1..37: mol_type = other RNA
attgctccttacgaggagacataacttgatcaagcag

SEQ ID NO: 1268 - RNA, 37 nt, synthetic construct
  source 1..37: mol_type = other RNA
attgctccttacgaggagacggagatggcagtttcct

SEQ ID NO: 1269 - RNA, 37 nt, synthetic construct
  source 1..37: mol_type = other RNA
attgctccttacgaggagacctctggtgacacaacct

SEQ ID NO: 1270 - RNA, 37 nt, synthetic construct
  source 1..37: mol_type = other RNA
attgctccttacgaggagacatgttggaggtacctgc

SEQ ID NO: 1271 - RNA, 37 nt, synthetic construct
  source 1..37: mol_type = other RNA
attgctccttacgaggagacctctgcttgatcaagtt

SEQ ID NO: 1272 - RNA, 37 nt, synthetic construct
  source 1..37: mol_type = other RNA
attgctccttacgaggagactctgcttgatcaagtta

SEQ ID NO: 1273 - RNA, 37 nt, synthetic construct
  source 1..37: mol_type = other RNA
attgctccttacgaggagaccttgatgttggaggtac

SEQ ID NO: 1274 - RNA, 37 nt, synthetic construct
  source 1..37: mol_type = other RNA
attgctccttacgaggagacaaccgggcttggacaga

SEQ ID NO: 1275 - RNA, 37 nt, synthetic construct
  source 1..37: mol_type = other RNA
attgctccttacgaggagacaggatatcaacgagatg

SEQ ID NO: 1276 - RNA, 37 nt, synthetic construct
  source 1..37: mol_type = other RNA
attgctccttacgaggagacgaggtacctgctctggc

SEQ ID NO: 1277 - RNA, 37 nt, synthetic construct
  source 1..37: mol_type = other RNA
attgctccttacgaggagaccaaccgggcttggacag

SEQ ID NO: 1278 - RNA, 37 nt, synthetic construct
  source 1..37: mol_type = other RNA
attgctccttacgaggagacctaaggaaactgccatc

SEQ ID NO: 1279 - RNA, 37 nt, synthetic construct
  source 1..37: mol_type = other RNA
attgctccttacgaggagactaaaatcacagagggtg

SEQ ID NO: 1280 - RNA, 37 nt, synthetic construct
  source 1..37: mol_type = other RNA
attgctccttacgaggagacgacagaacttaccgact

SEQ ID NO: 1281 - RNA, 37 nt, synthetic construct
  source 1..37: mol_type = other RNA
attgctccttacgaggagacccgactggctttctctg

SEQ ID NO: 1282 - RNA, 37 nt, synthetic construct
  source 1..37: mol_type = other RNA
attgctccttacgaggagacccgccttccactcagag

SEQ ID NO: 1283 - RNA, 37 nt, synthetic construct
  source 1..37: mol_type = other RNA
attgctccttacgaggagacccctcagctcttgaagt

SEQ ID NO: 1284 - RNA, 37 nt, synthetic construct
  source 1..37: mol_type = other RNA
attgctccttacgaggagacaagtaaacggtttaccg

SEQ ID NO: 1285 - RNA, 37 nt, synthetic construct
  source 1..37: mol_type = other RNA
attgctccttacgaggagacaccgccttccactcaga

SEQ ID NO: 1286 - RNA, 37 nt, synthetic construct
  source 1..37: mol_type = other RNA
attgctccttacgaggagacgccctcagctcttgaag

SEQ ID NO: 1287 - RNA, 37 nt, synthetic construct
  source 1..37: mol_type = other RNA
attgctccttacgaggagaccactcagagctcagatc

SEQ ID NO: 1288 - RNA, 37 nt, synthetic construct
  source 1..37: mol_type = other RNA
attgctccttacgaggagaccttcaagagctgagggc

SEQ ID NO: 1289 - RNA, 37 nt, synthetic construct
  source 1..37: mol_type = other RNA
attgctccttacgaggagacacttcaagagctgaggg

SEQ ID NO: 1290 - RNA, 37 nt, synthetic construct
  source 1..37: mol_type = other RNA
attgctccttacgaggagacaagagctgagggcaaag

SEQ ID NO: 1291 - RNA, 37 nt, synthetic construct
  source 1..37: mol_type = other RNA
attgctccttacgaggagactcctgtagaatactggc

SEQ ID NO: 1292 - RNA, 37 nt, synthetic construct
  source 1..37: mol_type = other RNA
attgctccttacgaggagacgaggattgctgaattat

SEQ ID NO: 1293 - RNA, 37 nt, synthetic construct
  source 1..37: mol_type = other RNA
attgctccttacgaggagacgccgctgcccaatgcca

SEQ ID NO: 1294 - RNA, 37 nt, synthetic construct
  source 1..37: mol_type = other RNA
attgctccttacgaggagacttccccagttgcattca

SEQ ID NO: 1295 - RNA, 37 nt, synthetic construct
  source 1..37: mol_type = other RNA
attgctccttacgaggagactgaggattgctgaatta

SEQ ID NO: 1296 - RNA, 37 nt, synthetic construct
  source 1..37: mol_type = other RNA
attgctccttacgaggagacccgctgcccaatgccat

SEQ ID NO: 1297 - RNA, 37 nt, synthetic construct
  source 1..37: mol_type = other RNA
attgctccttacgaggagacaggattgctgaattatt

SEQ ID NO: 1298 - RNA, 37 nt, synthetic construct
  source 1..37: mol_type = other RNA
attgctccttacgaggagacccaatttttcctgtaga

SEQ ID NO: 1299 - RNA, 37 nt, synthetic construct
  source 1..37: mol_type = other RNA
attgctccttacgaggagacttcctgtagaatactgg

SEQ ID NO: 1300 - RNA, 37 nt, synthetic construct
  source 1..37: mol_type = other RNA
attgctccttacgaggagacgcagacctcctgccacc

SEQ ID NO: 1301 - RNA, 37 nt, synthetic construct
  source 1..37: mol_type = other RNA
attgctccttacgaggagaccattcaatgttctgaca

SEQ ID NO: 1302 - RNA, 37 nt, synthetic construct
  source 1..37: mol_type = other RNA
attgctccttacgaggagactgacaacagtttgccgc

SEQ ID NO: 1303 - RNA, 37 nt, synthetic construct
  source 1..37: mol_type = other RNA
attgctccttacgaggagaccttccccagttgcattc

SEQ ID NO: 1304 - RNA, 37 nt, synthetic construct
  source 1..37: mol_type = other RNA
attgctccttacgaggagaccctgtagaatactggca

SEQ ID NO: 1305 - RNA, 37 nt, synthetic construct
  source 1..37: mol_type = other RNA
attgctccttacgaggagacctgaattatttcttccc

SEQ ID NO: 1306 - RNA, 37 nt, synthetic construct
  source 1..37: mol_type = other RNA
attgctccttacgaggagacctgtagaatactggcat

SEQ ID NO: 1307 - RNA, 37 nt, synthetic construct
  source 1..37: mol_type = other RNA
attgctccttacgaggagacttgaggattgctgaatt

SEQ ID NO: 1308 - RNA, 37 nt, synthetic construct
  source 1..37: mol_type = other RNA
attgctccttacgaggagacaatgttctgacaacagt

SEQ ID NO: 1309 - RNA, 37 nt, synthetic construct
  source 1..37: mol_type = other RNA
attgctccttacgaggagaccccagttgcattcaatg

SEQ ID NO: 1310 - RNA, 37 nt, synthetic construct
  source 1..37: mol_type = other RNA
attgctccttacgaggagaccagacctcctgccaccg

SEQ ID NO: 1311 - RNA, 37 nt, synthetic construct
  source 1..37: mol_type = other RNA
attgctccttacgaggagactttcttccccagttgca

SEQ ID NO: 1312 - RNA, 37 nt, synthetic construct
  source 1..37: mol_type = other RNA
attgctccttacgaggagacaggcttcccaatttttc

SEQ ID NO: 1313 - RNA, 37 nt, synthetic construct
  source 1..37: mol_type = other RNA
attgctccttacgaggagacggaagcctgaatctgcg

SEQ ID NO: 1314 - RNA, 37 nt, synthetic construct
  source 1..37: mol_type = other RNA
attgctccttacgaggagacagcaatcctcaaaaaca

SEQ ID NO: 1315 - RNA, 37 nt, synthetic construct
  source 1..37: mol_type = other RNA
attgctccttacgaggagactcagaacattgaatgca

SEQ ID NO: 1316 - RNA, 37 nt, synthetic construct
  source 1..37: mol_type = other RNA
attgctccttacgaggagacaatgcaactggggaaga

SEQ ID NO: 1317 - RNA, 37 nt, synthetic construct
  source 1..37: mol_type = other RNA
attgctccttacgaggagacggcagcggcaaactgtt

SEQ ID NO: 1318 - RNA, 37 nt, synthetic construct
  source 1..37: mol_type = other RNA
attgctccttacgaggagactacaggaaaaattggga

SEQ ID NO: 1319 - RNA, 37 nt, synthetic construct
  source 1..37: mol_type = other RNA
attgctccttacgaggagacgccactgattaaatatc

SEQ ID NO: 1320 - RNA, 37 nt, synthetic construct
  source 1..37: mol_type = other RNA
attgctccttacgaggagacgtgtctttctgagaaac

SEQ ID NO: 1321 - RNA, 37 nt, synthetic construct
  source 1..37: mol_type = other RNA
attgctccttacgaggagactcaacagatctgtcaaa

SEQ ID NO: 1322 - RNA, 37 nt, synthetic construct
  source 1..37: mol_type = other RNA
attgctccttacgaggagacgtatttagcatgttccc

SEQ ID NO: 1323 - RNA, 37 nt, synthetic construct
  source 1..37: mol_type = other RNA
attgctccttacgaggagactatttagcatgttccca

SEQ ID NO: 1324 - RNA, 37 nt, synthetic construct
  source 1..37: mol_type = other RNA
attgctccttacgaggagactgttagccactgattaa

SEQ ID NO: 1325 - RNA, 37 nt, synthetic construct
  source 1..37: mol_type = other RNA
attgctccttacgaggagactgagaaactgttcagct

SEQ ID NO: 1326 - RNA, 37 nt, synthetic construct
  source 1..37: mol_type = other RNA
attgctccttacgaggagacagcatgttcccaattct

SEQ ID NO: 1327 - RNA, 37 nt, synthetic construct
  source 1..37: mol_type = other RNA
attgctccttacgaggagactatcataatgaaaacgc

SEQ ID NO: 1328 - RNA, 37 nt, synthetic construct
  source 1..37: mol_type = other RNA
attgctccttacgaggagacctgagaaactgttcagc

SEQ ID NO: 1329 - RNA, 37 nt, synthetic construct
  source 1..37: mol_type = other RNA
attgctccttacgaggagacccaattctcaggaattt

SEQ ID NO: 1330 - RNA, 37 nt, synthetic construct
  source 1..37: mol_type = other RNA
attgctccttacgaggagactgtctttctgagaaact

SEQ ID NO: 1331 - RNA, 37 nt, synthetic construct
  source 1..37: mol_type = other RNA
attgctccttacgaggagactcaggaatttgtgtctt

SEQ ID NO: 1332 - RNA, 37 nt, synthetic construct
  source 1..37: mol_type = other RNA
attgctccttacgaggagacgcatgttcccaattctc

SEQ ID NO: 1333 - RNA, 37 nt, synthetic construct
  source 1..37: mol_type = other RNA
attgctccttacgaggagacagcttctgttagccact

SEQ ID NO: 1334 - RNA, 37 nt, synthetic construct
  source 1..37: mol_type = other RNA
attgctccttacgaggagactcagaaagacacaaatt

SEQ ID NO: 1335 - RNA, 37 nt, synthetic construct
  source 1..37: mol_type = other RNA
attgctccttacgaggagacaatcagtggctaacaga

SEQ ID NO: 1336 - RNA, 37 nt, synthetic construct
  source 1..37: mol_type = other RNA
attgctccttacgaggagacagaaatggcggcgtttt

SEQ ID NO: 1337 - RNA, 37 nt, synthetic construct
  source 1..37: mol_type = other RNA
attgctccttacgaggagacatcagtggctaacagaa

SEQ ID NO: 1338 - RNA, 37 nt, synthetic construct
  source 1..37: mol_type = other RNA
attgctccttacgaggagacggaacatgctaaataca

SEQ ID NO: 1339 - RNA, 37 nt, synthetic construct
  source 1..37: mol_type = other RNA
attgctccttacgaggagacctgagaattgggaacat

SEQ ID NO: 1340 - RNA, 37 nt, synthetic construct
  source 1..37: mol_type = other RNA
attgctccttacgaggagacctcagaaagacacaaat

SEQ ID NO: 1341 - RNA, 41 nt, synthetic construct
  source 1..41: mol_type = other RNA
atagattgctccttacgaggagacgtatcttacaggaactc

SEQ ID NO: 1342 - RNA, 41 nt, synthetic construct
  source 1..41: mol_type = other RNA
atagattgctccttacgaggagactggtatcttacaggaac

SEQ ID NO: 1343 - RNA, 37 nt, synthetic construct
  source 1..37: mol_type = other RNA
  modified_base 35: mod_base = OTHER; note = 2'-O-Methyl cytidine
  modified_base 36: mod_base = OTHER; note = 2'-O-Methyl adenosine
  modified_base 37: mod_base = OTHER; note = 2'-O-Methyl guanosine
atagattgctccttacgaggagactggtatcttacag

SEQ ID NO: 1344 - RNA, 103 nt, synthetic construct
  source 1..103: mol_type = other RNA
tggggcagttggttgcccttagcctgaggcatttattgcactcgggaagtaccatttctcagaaatggtacatccaacaaattgtatttatcttcagctttta

SEQ ID NO: 1345 - RNA, 103 nt, synthetic construct
  source 1..103: mol_type = other RNA
tggggcagttggttgcccttagcctgaggcatttattgcactcgggaagtaccatttctcagaaatggtacatccaacaaaaaacaaatcaaagactttttta

SEQ ID NO: 1346 - RNA, 103 nt, synthetic construct
  source 1..103: mol_type = other RNA
tggggcagttggttgcccttagcctgaggcatttattgcactcgggaagtaccatttctcagaaatggtacatccaaccctgcaggtaaaagcatatgtttta

SEQ ID NO: 1347 - RNA, 103 nt, synthetic construct
  source 1..103: mol_type = other RNA
tggggcagttggttgcccttagcctgaggcatttattgcactcgggaagtaccatttctcagaaatggtacatccaacccctacctcttttttctgtctttta

SEQ ID NO: 1348 - RNA, 103 nt, synthetic construct
  source 1..103: mol_type = other RNA
tggggcagttggttgcccttagcctgaggcatttattgcactcgggaagtaccatttctcagaaatggtacatccaacaataagtaatgtgtatgctttttta

SEQ ID NO: 1349 - RNA, 103 nt, synthetic construct
  source 1..103: mol_type = other RNA
tggggcagttggttgcccttagcctgaggcatttattgcactcgggaagtaccatttctcagaaatggtacatccaacattaacactttgaagataattttta

SEQ ID NO: 1350 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacgcctgggtgacagtgagacttttta

SEQ ID NO: 1351 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacttttaaaaaggtatctttgatttta

SEQ ID NO: 1352 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacacgtgattttctgttaataatttta

SEQ ID NO: 1353 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaaccattaaacatcattaaattatttta

SEQ ID NO: 1354 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacatactaaccttggtttctgttttta

SEQ ID NO: 1355 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacaaaaggtatctttgatactatttta

SEQ ID NO: 1356 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaactcaaatgtaaccagtatttttttta

SEQ ID NO: 1357 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacatgtaaagttattaacagaatttta

SEQ ID NO: 1358 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacttttcttagagacagagtcttttta

SEQ ID NO: 1359 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaactatatttattttcttagagatttta

SEQ ID NO: 1360 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacaaataaaatactggttacattttta

SEQ ID NO: 1361 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacatgatgtttaatgtaaagtttttta

SEQ ID NO: 1362 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacataaaattataccatagatttttta

SEQ ID NO: 1363 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacatggcaaatattagtttctgtttta

SEQ ID NO: 1364 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacaatgtaacttccaaacgttatttta

SEQ ID NO: 1365 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacaaaaattattcattgtgttatttta

SEQ ID NO: 1366 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaactgttgcttatttaaaaaatttttta

SEQ ID NO: 1367 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaaccttttttgatggcaaatatttttta

SEQ ID NO: 1368 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacacagaaactaatatttgccatttta

SEQ ID NO: 1369 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacaaatgtctcctccagactagtttta

SEQ ID NO: 1370 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacccatcaaaaaagcaaagaattttta

SEQ ID NO: 1371 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacaataagcaacataaatgtgatttta

SEQ ID NO: 1372 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaactttttccttttattctagtttttta

SEQ ID NO: 1373 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacttctagttgaaagaattcagtttta

SEQ ID NO: 1374 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacctactatatatttatttttctttta

SEQ ID NO: 1375 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacgaagttacatttaaaatgtctttta

SEQ ID NO: 1376 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaactatccttgattatacttaggtttta

SEQ ID NO: 1377 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaaccattaatttatatccttgattttta

SEQ ID NO: 1378 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacaagaaaaacttctgccaacttttta

SEQ ID NO: 1379 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaactcattttttctcataccttctttta

SEQ ID NO: 1380 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacttatttgcattaatttatattttta

SEQ ID NO: 1381 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacctgagagagaaacagttgcctttta

SEQ ID NO: 1382 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacctctcctagaccatttcccatttta

SEQ ID NO: 1383 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacaaatgaagattttccaccaatttta

SEQ ID NO: 1384 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacagccaaactcttattcatgatttta

SEQ ID NO: 1385 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacggtttttgcaaaaaggaaaatttta

SEQ ID NO: 1386 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacgcaatacatggtagaaaatgtttta

SEQ ID NO: 1387 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaactttagcaatacatggtagaatttta

SEQ ID NO: 1388 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacattgaagagtaacaatttgatttta

SEQ ID NO: 1389 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaaccaaaaaggaaaaaagaagaatttta

SEQ ID NO: 1390 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacgcttgtgtttctaatttttctttta

SEQ ID NO: 1391 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacgctcaaattgttactcttcatttta

SEQ ID NO: 1392 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacacttattgttattgaaattgtttta

SEQ ID NO: 1393 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacgctcctactcagactgttactttta

SEQ ID NO: 1394 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaaccaaaaacccaaaatattttatttta

SEQ ID NO: 1395 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaaccacagtcaataacacaaaggtttta

SEQ ID NO: 1396 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacaacaaatagctagagccaaatttta

SEQ ID NO: 1397 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacaatcagagtcaatttccaagtttta

SEQ ID NO: 1398 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaactctctatctttagaattgaatttta

SEQ ID NO: 1399 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacgaattgaaacaaattttctctttta

SEQ ID NO: 1400 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacgctctagctatttgttcaaatttta

SEQ ID NO: 1401 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaactttcaattctaaagatagagtttta

SEQ ID NO: 1402 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacttcaaaagtgcaactatgaatttta

SEQ ID NO: 1403 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaactgttattgactgtgcaaaaatttta

SEQ ID NO: 1404 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacgtgggtagaatttcttttaatttta

SEQ ID NO: 1405 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacgtgaatatattattggattttttta

SEQ ID NO: 1406 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacacagaaaagcatacacattatttta

SEQ ID NO: 1407 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaaccttctaaattaactttagtgtttta

SEQ ID NO: 1408 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacaagataattcatgaacatcttttta

SEQ ID NO: 1409 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacgccaaccaccctacaaatattttta

SEQ ID NO: 1410 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacaaggaattataattattttatttta

SEQ ID NO: 1411 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacgaagtaaaatataatagaaatttta

SEQ ID NO: 1412 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaactagggtggttggctaaaatatttta

SEQ ID NO: 1413 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacaaagaaattctacccactaatttta

SEQ ID NO: 1414 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacctgctgttgattaatggttgtttta

SEQ ID NO: 1415 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacatgttagtgcctttcacccttttta

SEQ ID NO: 1416 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacgtatatgaaaaaaagaaatatttta

SEQ ID NO: 1417 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacagctttctttagaagaatattttta

SEQ ID NO: 1418 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacgaagaatatttcatgagagatttta

SEQ ID NO: 1419 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacgcagactttttaagctttcttttta

SEQ ID NO: 1420 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaactttctttttttcatatactatttta

SEQ ID NO: 1421 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacagaaaagattaaacagtgtgtttta

SEQ ID NO: 1422 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaactttgagaaaagattaaacagtttta

SEQ ID NO: 1423 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacttttgcctttttggtatctttttta

SEQ ID NO: 1424 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaactatttctttctttgccagtatttta

SEQ ID NO: 1425 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacgtatcttacaggaactccagtttta

SEQ ID NO: 1426 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaaccctttttggtatcttacaggtttta

SEQ ID NO: 1427 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacccagtacaactgcatgtggttttta

SEQ ID NO: 1428 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacgatatgggcatgtcagtttctttta

SEQ ID NO: 1429 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacatcttttctcaaataaaaagtttta

SEQ ID NO: 1430 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacagaagttaaagagtccagattttta

SEQ ID NO: 1431 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacatttgttttttcgaaattgttttta

SEQ ID NO: 1432 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacactgttgttgtcatcattattttta

SEQ ID NO: 1433 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacttttttcgaaattgtatttatttta

SEQ ID NO: 1434 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacacttcttaaagatcaggttctttta

SEQ ID NO: 1435 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaactcttcagcacatctggactctttta

SEQ ID NO: 1436 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaaccagagtttagtttcaagtaatttta

SEQ ID NO: 1437 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacgtttcaagtaacaaataaaatttta

SEQ ID NO: 1438 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacgttatactgacaaagatatctttta

SEQ ID NO: 1439 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaactttgttacttgaaactaaactttta

SEQ ID NO: 1440 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacttacttgaaactaaactctgtttta

SEQ ID NO: 1441 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacacagatctgttgagaaatggtttta

SEQ ID NO: 1442 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaaccataatccatctatttttcttttta

SEQ ID NO: 1443 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaaccctgcaggcgatttgacagatttta

SEQ ID NO: 1444 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaacaaatgttgtgtgtacatgcttttta

SEQ ID NO: 1445 - RNA, 166 nt, synthetic construct
  source 1..166: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaactcagtataaccaaaaaatattttta

SEQ ID NO: 1446 - RNA, 160 nt, synthetic construct
  source 1..160: mol_type = other RNA
accgcttcaccaagtgctgtcccttagggattagcacttgagtgaaggtgggctgcttgcatcagcctaatgtcgagaagtgctttcttcggaaagtaaccctcgaaacaaattcatttgaaagaatgaaggaatgcaacacatttcattcaactgttgc

SEQ ID NO: 1447 - RNA, 160 nt, synthetic construct
  source 1..160: mol_type = other RNA
accgcttcaccaagtgctgtcccttagggattagcacttgagtgaaggtgggctgcttgcatcagcctaatgtcgagaagtgctttcttcggaaagtaaccctcgaaacaaattcatttgaaagaatgaaggaatgcaacgattgcatctactgtatagg

SEQ ID NO: 1448 - RNA, 160 nt, synthetic construct
  source 1..160: mol_type = other RNA
accgcttcaccaagtgctgtcccttagggattagcacttgagtgaaggtgggctgcttgcatcagcctaatgtcgagaagtgctttcttcggaaagtaaccctcgaaacaaattcatttgaaagaatgaaggaatgcaactaacttgatcaagcagagaa

SEQ ID NO: 1449 - RNA, 160 nt, synthetic construct
  source 1..160: mol_type = other RNA
accgcttcaccaagtgctgtcccttagggattagcacttgagtgaaggtgggctgcttgcatcagcctaatgtcgagaagtgctttcttcggaaagtaaccctcgaaacaaattcatttgaaagaatgaaggaatgcaacgagatggcagtttccttagt

SEQ ID NO: 1450 - RNA, 160 nt, synthetic construct
  source 1..160: mol_type = other RNA
accgcttcaccaagtgctgtcccttagggattagcacttgagtgaaggtgggctgcttgcatcagcctaatgtcgagaagtgctttcttcggaaagtaaccctcgaaacaaattcatttgaaagaatgaaggaatgcaacccctcagctcttgaagtaaa

SEQ ID NO: 1451 - RNA, 160 nt, synthetic construct
  source 1..160: mol_type = other RNA
accgcttcaccaagtgctgtcccttagggattagcacttgagtgaaggtgggctgcttgcatcagcctaatgtcgagaagtgctttcttcggaaagtaaccctcgaaacaaattcatttgaaagaatgaaggaatgcaacccgccttccactcagagctc

SEQ ID NO: 1452 - RNA, 160 nt, synthetic construct
  source 1..160: mol_type = other RNA
accgcttcaccaagtgctgtcccttagggattagcacttgagtgaaggtgggctgcttgcatcagcctaatgtcgagaagtgctttcttcggaaagtaaccctcgaaacaaattcatttgaaagaatgaaggaatgcaaccttcaagagctgagggcaaa

SEQ ID NO: 1453 - RNA, 160 nt, synthetic construct
  source 1..160: mol_type = other RNA
accgcttcaccaagtgctgtcccttagggattagcacttgagtgaaggtgggctgcttgcatcagcctaatgtcgagaagtgctttcttcggaaagtaaccctcgaaacaaattcatttgaaagaatgaaggaatgcaaccagacctcctgccaccgcag

SEQ ID NO: 1454 - RNA, 160 nt, synthetic construct
  source 1..160: mol_type = other RNA
accgcttcaccaagtgctgtcccttagggattagcacttgagtgaaggtgggctgcttgcatcagcctaatgtcgagaagtgctttcttcggaaagtaaccctcgaaacaaattcatttgaaagaatgaaggaatgcaacccgctgcccaatgccatcct

SEQ ID NO: 1455 - RNA, 160 nt, synthetic construct
  source 1..160: mol_type = other RNA
accgcttcaccaagtgctgtcccttagggattagcacttgagtgaaggtgggctgcttgcatcagcctaatgtcgagaagtgctttcttcggaaagtaaccctcgaaacaaattcatttgaaagaatgaaggaatgcaacaggattgctgaattatttct

SEQ ID NO: 1456 - RNA, 160 nt, synthetic construct
  source 1..160: mol_type = other RNA
accgcttcaccaagtgctgtcccttagggattagcacttgagtgaaggtgggctgcttgcatcagcctaatgtcgagaagtgctttcttcggaaagtaaccctcgaaacaaattcatttgaaagaatgaaggaatgcaacgcatgttcccaattctcagg

SEQ ID NO: 1457 - RNA, 160 nt, synthetic construct
  source 1..160: mol_type = other RNA
accgcttcaccaagtgctgtcccttagggattagcacttgagtgaaggtgggctgcttgcatcagcctaatgtcgagaagtgctttcttcggaaagtaaccctcgaaacaaattcatttgaaagaatgaaggaatgcaactatcataatgaaaacgccgc

SEQ ID NO: 1458 - RNA, 160 nt, synthetic construct
  source 1..160: mol_type = other RNA
accgcttcaccaagtgctgtcccttagggattagcacttgagtgaaggtgggctgcttgcatcagcctaatgtcgagaagtgctttcttcggaaagtaaccctcgaaacaaattcatttgaaagaatgaaggaatgcaactgtctttctgagaaactgtt

SEQ ID NO: 1459 - RNA, 160 nt, synthetic construct
  source 1..160: mol_type = other RNA
accgcttcaccaagtgctgtcccttagggattagcacttgagtgaaggtgggctgcttgcatcagcctaatgtcgagaagtgctttcttcggaaagtaaccctcgaaacaaattcatttgaaagaatgaaggaatgcaactatttagcatgttcccaatt

SEQ ID NO: 1460 - RNA, 160 nt, synthetic construct
  source 1..160: mol_type = other RNA
accgcttcaccaagtgctgtcccttagggattagcacttgagtgaaggtgggctgcttgcatcagcctaatgtcgagaagtgctttcttcggaaagtaaccctcgaaacaaattcatttgaaagaatgaaggaatgcaacatcagtggctaacagaagct

SEQ ID NO: 1461 - protein, 3685 aa, Homo sapiens
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQAVTTTQPSLTQTTVMETVTTVTTREQILVKHAQEELPPPPPQKKRQITVDSEIRKRLDVDITELHSWITRSEAVLQSPEFAIFRKEGNFSDLKEKVNAIEREKAEKFRKLQDASRSAQALVEQMVNEGVNADSIKQASEQLNSRWIEFCQLLSERLNWLEYQNNIIAFYNQLQQLEQMTTTAENWLKIQPTTPSEPTAIKSQLKICKDEVNRLSDLQPQIERLKIQSIALKEKGQGPMFLDADFVAFTNHFKQVFSDVQAREKELQTIFDTLPPMRYQETMSAIRTWVQQSETKLSIPQLSVTDYEIMEQRLGELQALQSSLQEQQSGLYYLSTTVKEMSKKAPSEISRKYQSEFEEIEGRWKKLSSQLVEHCQKLEEQMNKLRKIQNHIQTLKKWMAEVDVFLKEEWPALGDSEILKKQLKQCRLLVSDIQTIQPSLNSVNEGGQKIKNEAEPEFASRLETELKELNTQWDHMCQQVYARKEALKGGLEKTVSLQKDLSEMHEWMTQAEEEYLERDFEYKTPDELQKAVEEMKRAKEEAQQKEAKVKLLTESVNSVIAQAPPVAQEALKKELETLTTNYQWLCTRLNGKCKTLEEVWACWHELLSYLEKANKWLNEVEFKLKTTENIPGGAEEISEVLDSLENLMRHSEDNPNQIRILAQTLTDGGVMDELINEELETFNSRWRELHEEAVRRQKLLEQSIQSAQETEKSLHLIQESLTFIDKQLAAYIADKVDAAQMPQEAQKIQSDLTSHEISLEEMKKHNQGKEAAQRVLSQIDVAQKKLQDVSMKFRLFQKPANFEQRLQESKMILDEVKMHLPALETKSVEQEVVQSQLNHCVNLYKSLSEVKSEVEMVIKTGRQIVQKKQTENPKELDERVTALKLHYNELGAKVTERKQQLEKCLKLSRKMRKEMNVLTEWLAATDMELTKRSAVEGMPSNLDSEVAWGKATQKEIEKQKVHLKSITEVGEALKTVLGKKETLVEDKLSLLNSNWIAVTSRAEEWLNLLLEYQKHMETFDQNVDHITKWIIQADTLLDESEKKKPQQKEDVLKRLKAELNDIRPKVDSTRDQAANLMANRGDHCRKLVEPQISELNHRFAAISHRIKTGKASIPLKELEQFNSDIQKLLEPLEAEIQQGVNLKEEDFNKDMNEDNEGTVKELLQRGDNLQQRITDERKREEIKIKQQLLQTKHNALKDLRSQRRKKALEISHQWYQYKRQADDLLKCLDDIEKKLASLPEPRDERKIKEIDRELQKKKEELNAVRRQAEGLSEDGAAMAVEPTQIQLSKRWREIESKFAQFRRLNFAQIHTVREETMMVMTEDMPLEISYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQKNILSEFQRDLNEFVLWLEEADNIASIPLEPGKEQQLKEKLEQVKLLVEELPLRQGILKQLNETGGPVLVSAPISPEEQDKLENKLKQTNLQWIKVSRALPEKQGEIEAQIKDLGQLEKKLEDLEEQLNHLLLWLSPIRNQLEIYNQPNQEGPFDVKETEIAVQAKQPDVEEILSKGQHLYKEKPATQPVKRKLEDLSSEWKAVNRLLQELRAKQPDLAPGLTTIGASPTQTVTLVTQPVVTKETAISKLEMPSSLMLEVPALADFNRAWTELTDWLSLLDQVIKSQRVMVGDLEDINEMIIKQKATMQDLEQRRPQLEELITAAQNLKNKTSNQEARTIITDRIERIQNQWDEVQEHLQNRRQQLNEMLKDSTQWLEAKEEAEQVLGQARAKLESWKEGPYTVDAIQKKITETKQLAKDLRQWQTNVDVANDLALKLLRDYSADDTRKVHMITENINASWRSIHKRVSEREAALEETHRLLQQFPLDLEKFLAWLTEAETTANVLQDATRKERLLEDSKGVKELMKQWQDLQGEIEAHTDVYHNLDENSQKILRSLEGSDDAVLLQRRLDNMNFKWSELRKKSLNIRSHLEASSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPRDAELIAEAKLLRQHKGRLEARMQILEDHNKQLESQLHRLRQLLEQPQAEAKVNGTTVSSPSTSLQRSDSSQPMLLRVVGSQTSDSMGEEDLLSPPQDTSTGLEEVMEQLNNSFPSSRGRNTPGKPMREDTM

SEQ ID NO: 1462 - DNA, 13992 bp, Homo sapiens
  source 1..13992: mol_type = genomic DNA
atcagttactgtgttgactcactcagtgttgggatcactcactttccccctacaggactcagatctgggaggcaattaccttcggagaaaaacgaataggaaaaactgaagtgttactttttttaaagctgctgaagtttgttggtttctcattgtttttaagcctactggagcaataaagtttgaagaacttttaccaggttttttttatcgctgccttgatatacacttttcaaaatgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatctccagaatcagaaactgaaagagttgaatgactggctaacaaaaacagaagaaagaacaaggaaaatggaggaagagcctcttggacctgatcttgaagacctaaaacgccaagtacaacaacataaggtgcttcaagaagatctagaacaagaacaagtcagggtcaattctctcactcacatggtggtggtagttgatgaatctagtggagatcacgcaactgctgctttggaagaacaacttaaggtattgggagatcgatgggcaaacatctgtagatggacagaagaccgctgggttcttttacaagacatccttctcaaatggcaacgtcttactgaagaacagtgcctttttagtgcatggctttcagaaaaagaagatgcagtgaacaagattcacacaactggctttaaagatcaaaatgaaatgttatcaagtcttcaaaaactggccgttttaaaagcggatctagaaaagaaaaagcaatccatgggcaaactgtattcactcaaacaagatcttctttcaacactgaagaataagtcagtgacccagaagacggaagcatggctggataactttgcccggtgttgggataatttagtccaaaaacttgaaaagagtacagcacagatttcacaggctgtcaccaccactcagccatcactaacacagacaactgtaatggaaacagtaactacggtgaccacaagggaacagatcctggtaaagcatgctcaagaggaacttccaccaccacctccccaaaagaagaggcagattactgtggattctgaaattaggaaaaggttggatgttgatataactgaacttcacagctggattactcgctcagaagctgtgttgcagagtcctgaatttgcaatctttcggaaggaaggcaacttctcagacttaaaagaaaaagtcaatgccatagagcgagaaaaagctgagaagttcagaaaactgcaagatgccagcagatcagctcaggccctggtggaacagatggtgaatgagggtgttaatgcagatagcatcaaacaagcctcagaacaactgaacagccggtggatcgaattctgccagttgctaagtgagagacttaactggctggagtatcagaacaacatcatcgctttctataatcagctacaacaattggagcagatgacaactactgctgaaaactggttgaaaatccaacccaccaccccatcagagccaacagcaattaaaagtcagttaaaaatttgtaaggatgaagtcaaccggctatcagatcttcaacctcaaattgaacgattaaaaattcaaagcatagccctgaaagagaaaggacaaggacccatgttcctggatgcagactttgtggcctttacaaatcattttaagcaagtcttttctgatgtgcaggccagagagaaagagctacagacaatttttgacactttgccaccaatgcgctatcaggagaccatgagtgccatcaggacatgggtccagcagtcagaaaccaaactctccatacctcaacttagtgtcaccgactatgaaatcatggagcagagactcggggaattgcaggctttacaaagttctctgcaagagcaacaaagtggcctatactatctcagcaccactgtgaaagagatgtcgaagaaagcgccctctgaaattagccggaaatatcaatcagaatttgaagaaattgagggacgctggaagaagctctcctcccagctggttgagcattgtcaaaagctagaggagcaaatgaataaactccgaaaaattcagaatcacatacaaaccctgaagaaatggatggctgaagttgatgtttttctgaaggaggaatggcctgcccttggggattcagaaattctaaaaaagcagctgaaacagtgcagacttttagtcagtgatattcagacaattcagcccagtctaaacagtgtcaatgaaggtgggcagaagataaagaatgaagcagagccagagtttgcttcgagacttgagacagaactcaaagaacttaacactcagtgggatcacatgtgccaacaggtctatgccagaaaggaggccttgaagggaggtttggagaaaactgtaagcctccagaaagatctatcagagatgcacgaatggatgacacaagctgaagaagagtatcttgagagagattttgaatataaaactccagatgaattacagaaagcagttgaagagatgaagagagctaaagaagaggcccaacaaaaagaagcgaaagtgaaactccttactgagtctgtaaatagtgtcatagctcaagctccacctgtagcacaagaggccttaaaaaaggaacttgaaactctaaccaccaactaccagtggctctgcactaggctgaatgggaaatgcaagactttggaagaagtttgggcatgttggcatgagttattgtcatacttggagaaagcaaacaagtggctaaatgaagtagaatttaaacttaaaaccactgaaaacattcctggcggagctgaggaaatctctgaggtgctagattcacttgaaaatttgatgcgacattcagaggataacccaaatcagattcgcatattggcacagaccctaacagatggcggagtcatggatgagctaatcaatgaggaacttgagacatttaattctcgttggagggaactacatgaagaggctgtaaggaggcaaaagttgcttgaacagagcatccagtctgcccaggagactgaaaaatccttacacttaatccaggagtccctcacattcattgacaagcagttggcagcttatattgcagacaaggtggacgcagctcaaatgcctcaggaagcccagaaaatccaatctgatttgacaagtcatgagatcagtttagaagaaatgaagaaacataatcaggggaaggaggctgcccaaagagtcctgtctcagattgatgttgcacagaaaaaattacaagatgtctccatgaagtttcgattattccagaaaccagccaattttgagcagcgtctacaagaaagtaagatgattttagatgaagtgaagatgcacttgcctgcattggaaacaaagagtgtggaacaggaagtagtacagtcacagctaaatcattgtgtgaacttgtataaaagtctgagtgaagtgaagtctgaagtggaaatggtgataaagactggacgtcagattgtacagaaaaagcagacggaaaatcccaaagaacttgatgaaagagtaacagctttgaaattgcattataatgagctgggagcaaaggtaacagaaagaaagcaacagttggagaaatgcttgaaattgtcccgtaagatgcgaaaggaaatgaatgtcttgacagaatggctggcagctacagatatggaattgacaaagagatcagcagttgaaggaatgcctagtaatttggattctgaagttgcctggggaaaggctactcaaaaagagattgagaaacagaaggtgcacctgaagagtatcacagaggtaggagaggccttgaaaacagttttgggcaagaaggagacgttggtggaagataaactcagtcttctgaatagtaactggatagctgtcacctcccgagcagaagagtggttaaatcttttgttggaataccagaaacacatggaaacttttgaccagaatgtggaccacatcacaaagtggatcattcaggctgacacacttttggatgaatcagagaaaaagaaaccccagcaaaaagaagacgtgcttaagcgtttaaaggcagaactgaatgacatacgcccaaaggtggactctacacgtgaccaagcagcaaacttgatggcaaaccgcggtgaccactgcaggaaattagtagagccccaaatctcagagctcaaccatcgatttgcagccatttcacacagaattaagactggaaaggcctccattcctttgaaggaattggagcagtttaactcagatatacaaaaattgcttgaaccactggaggctgaaattcagcagggggtgaatctgaaagaggaagacttcaataaagatatgaatgaagacaatgagggtactgtaaaagaattgttgcaaagaggagacaacttacaacaaagaatcacagatgagagaaagcgagaggaaataaagataaaacagcagctgttacagacaaaacataatgctctcaaggatttgaggtctcaaagaagaaaaaaggctctagaaatttctcatcagtggtatcagtacaagaggcaggctgatgatctcctgaaatgcttggatgacattgaaaaaaaattagccagcctacctgagcccagagatgaaaggaaaataaaggaaattgatcgggaattgcagaagaagaaagaggagctgaatgcagtgcgtaggcaagctgagggcttgtctgaggatggggccgcaatggcagtggagccaactcagatccagctcagcaagcgctggcgggaaattgagagcaaatttgctcagtttcgaagactcaactttgcacaaattcacactgtccgtgaagaaacgatgatggtgatgactgaagacatgcctttggaaatttcttatgtgccttctacttatttgactgaaatcactcatgtctcacaagccctattagaagtggaacaacttctcaatgctcctgacctctgtgctaaggactttgaagatctctttaagcaagaggagtctctgaagaatataaaagatagtctacaacaaagctcaggtcggattgacattattcatagcaagaagacagcagcattgcaaagtgcaacgcctgtggaaagggtgaagctacaggaagctctctcccagcttgatttccaatgggaaaaagttaacaaaatgtacaaggaccgacaagggcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaaaagaatatcttgtcagaatttcaaagagatttaaatgaatttgttttatggttggaggaagcagataacattgctagtatcccacttgaacctggaaaagagcagcaactaaaagaaaagcttgagcaagtcaagttactggtggaagagttgcccctgcgccagggaattctcaaacaattaaatgaaactggaggacccgtgcttgtaagtgctcccataagcccagaagagcaagataaacttgaaaataagctcaagcagacaaatctccagtggataaaggtttccagagctttacctgagaaacaaggagaaattgaagctcaaataaaagaccttgggcagcttgaaaaaaagcttgaagaccttgaagagcagttaaatcatctgctgctgtggttatctcctattaggaatcagttggaaatttataaccaaccaaaccaagaaggaccatttgacgttaaggaaactgaaatagcagttcaagctaaacaaccggatgtggaagagattttgtctaaagggcagcatttgtacaaggaaaaaccagccactcagccagtgaagaggaagttagaagatctgagctctgagtggaaggcggtaaaccgtttacttcaagagctgagggcaaagcagcctgacctagctcctggactgaccactattggagcctctcctactcagactgttactctggtgacacaacctgtggttactaaggaaactgccatctccaaactagaaatgccatcttccttgatgttggaggtacctgctctggcagatttcaaccgggcttggacagaacttaccgactggctttctctgcttgatcaagttataaaatcacagagggtgatggtgggtgaccttgaggatatcaacgagatgatcatcaagcagaaggcaacaatgcaggatttggaacagaggcgtccccagttggaagaactcattaccgctgcccaaaatttgaaaaacaagaccagcaatcaagaggctagaacaatcattacggatcgaattgaaagaattcagaatcagtgggatgaagtacaagaacaccttcagaaccggaggcaacagttgaatgaaatgttaaaggattcaacacaatggctggaagctaaggaagaagctgagcaggtcttaggacaggccagagccaagcttgagtcatggaaggagggtccctatacagtagatgcaatccaaaagaaaatcacagaaaccaagcagttggccaaagacctccgccagtggcagacaaatgtagatgtggcaaatgacttggccctgaaacttctccgggattattctgcagatgataccagaaaagtccacatgataacagagaatatcaatgcctcttggagaagcattcataaaagggtgagtgagcgagaggctgctttggaagaaactcatagattactgcaacagttccccctggacctggaaaagtttcttgcctggcttacagaagctgaaacaactgccaatgtcctacaggatgctacccgtaaggaaaggctcctagaagactccaagggagtaaaagagctgatgaaacaatggcaagacctccaaggtgaaattgaagctcacacagatgtttatcacaacctggatgaaaacagccaaaaaatcctgagatccctggaaggttccgatgatgcagtcctgttacaaagacgtttggataacatgaacttcaagtggagtgaacttcggaaaaagtctctcaacattaggtcccatttggaagccagttctgaccagtggaagcgtctgcacctttctctgcaggaacttctggtgtggctacagctgaaagatgatgaattaagccggcaggcacctattggaggcgactttccagcagttcagaagcagaacgatgtacatagggccttcaagagggaattgaaaactaaagaacctgtaatcatgagtactcttgagactgtacgaatatttctgacagagcagcctttggaaggactagagaaactctaccaggagcccagagagctgcctcctgaggagagagcccagaatgtcactcggcttctacgaaagcaggctgaggaggtcaatactgagtgggaaaaattgaacctgcactccgctgactggcagagaaaaatagatgagacccttgaaagactccgggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactcccgttactctgatcaacttctggccagtagattctgcgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtccccactgccgtcccctcctgaaatgatgcccacctctccccagagtccccgggatgctgagctcattgctgaggccaagctactgcgtcaacacaaaggccgcctggaagccaggatgcaaatcctggaagaccacaataaacagctggagtcacagttacacaggctaaggcagctgctggagcaaccccaggcagaggccaaagtgaatggcacaacggtgtcctctccttctacctctctacagaggtccgacagcagtcagcctatgctgctccgagtggttggcagtcaaacttcggactccatgggtgaggaagatcttctcagtcctccccaggacacaagcacagggttagaggaggtgatggagcaactcaacaactccttccctagttcaagaggaagaaatacccctggaaagccaatgagagaggacacaatgtaggaagtcttttccacatggcagatgatttgggcagagcgatggagtccttagtatcagtcatgacagatgaagaaggagcagaataaatgttttacaactcctgattcccgcatggtttttataatattcatacaacaaagaggattagacagtaagagtttacaagaaataaatctatatttttgtgaagggtagtggtattatactgtagatttcagtagtttctaagtctgttattgttttgttaacaatggcaggttttacacgtctatgcaattgtacaaaaaagttataagaaaactacatgtaaaatcttgatagctaaataacttgccatttctttatatggaacgcattttgggttgtttaaaaatttataacagttataaagaaagattgtaaactaaagtgtgctttataaaaaaaagttgtttataaaaacccctaaaaacaaaacaaacacacacacacacacatacacacacacacacaaaactttgaggcagcgcattgttttgcatccttttggcgtgatatccatatgaaattcatggctttttctttttttgcatattaaagataagacttcctctaccaccacaccaaatgactactacacactgctcatttgagaactgtcagctgagtggggcaggcttgagttttcatttcatatatctatatgtctataagtatataaatactatagttatatagataaagagatacgaatttctatagactgactttttccattttttaaatgttcatgtcacatcctaatagaaagaaattacttctagtcagtcatccaggcttacctgcttggtctagaatggatttttcccggagccggaagccaggaggaaactacaccacactaaaacattgtctacagctccagatgtttctcattttaaacaactttccactgacaacgaaagtaaagtaaagtattggatttttttaaagggaacatgtgaatgaatacacaggacttattatatcagagtgagtaatcggttggttggttgattgattgattgattgatacattcagcttcctgctgctagcaatgccacgatttagatttaatgatgcttcagtggaaatcaatcagaaggtattctgaccttgtgaacatcagaaggtattttttaactcccaagcagtagcaggacgatgatagggctggagggctatggattcccagcccatccctgtgaaggagtaggccactctttaagtgaaggattggatgattgttcataatacataaagttctctgtaattacaactaaattattatgccctcttctcacagtcaaaaggaactgggtggtttggtttttgttgcttttttagatttattgtcccatgtgggatgagtttttaaatgccacaagacataatttaaaataaataaactttgggaaaaggtgtaaaacagtagccccatcacatttgtgatactgacaggtatcaacccagaagcccatgaactgtgtttccatcctttgcatttctctgcgagtagttccacacaggtttgtaagtaagtaagaaagaaggcaaattgattcaaatgttacaaaaaaacccttcttggtggattagacaggttaaatatataaacaaacaaacaaaaattgctcaaaaaagaggagaaaagctcaagaggaaaagctaaggactggtaggaaaaagctttactctttcatgccattttatttctttttgatttttaaatcattcattcaatagataccaccgtgtgacctataattttgcaaatctgttacctctgacatcaagtgtaattagcttttggagagtgggctgacatcaagtgtaattagcttttggagagtgggttttgtccattattaataattaattaattaacatcaaacacggcttctcatgctatttctacctcactttggttttggggtgttcctgataattgtgcacacctgagttcacagcttcaccacttgtccattgcgttattttctttttcctttataattctttctttttccttcataattttcaaaagaaaacccaaagctctaaggtaacaaattaccaaattacatgaagatttggtttttgtcttgcatttttttcctttatgtgacgctggaccttttctttacccaaggatttttaaaactcagatttaaaacaaggggttactttacatcctactaagaagtttaagtaagtaagtttcattctaaaatcagaggtaaatagagtgcataaataattttgttttaatctttttgtttttcttttagacacattagctctggagtgagtctgtcataatatttgaacaaaaattgagagctttattgctgcattttaagcataattaatttggacattatttcgtgttgtgttctttataaccaccaagtattaaactgtaaatcataatgtaactgaagcataaacatcacatggcatgttttgtcattgttttcaggtactgagttcttacttgagtatcataatatattgtgttttaacaccaacactgtaacatttacgaattatttttttaaacttcagttttactgcattttcacaacatatcagacttcaccaaatatatgccttactattgtattatagtactgctttactgtgtatctcaataaagcacgcagttatgttacaaaaaa

SEQ ID NO: 1463 - DNA, 148 bp, synthetic construct
  source 1..148: mol_type = other DNA
cttaagataccatttgtatttagcatgttcccaattctcaggaatttgtgtctttctgagaaactgttcagcttctgttagccactgattaaatatctttatatcataatgaaaacgccgccatttctcaacagatctgtcaaatcgc

SEQ ID NO: 1464 - DNA, 176 bp, synthetic construct
  source 1..176: mol_type = other DNA
gaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagag

SEQ ID NO: 1465 - DNA, 109 bp, synthetic construct
  source 1..109: mol_type = other DNA
aggaagttagaagatctgagctctgagtggaaggcggtaaaccgtttacttcaagagctgagggcaaagcagcctgacctagctcctggactgaccactattggagcct

SEQ ID NO: 1466 - DNA, 233 bp, synthetic construct
  source 1..233: mol_type = other DNA
ctcctactcagactgttactctggtgacacaacctgtggttactaaggaaactgccatctccaaactagaaatgccatcttccttgatgttggaggtacctgctctggcagatttcaaccgggcttggacagaacttaccgactggctttctctgcttgatcaagttataaaatcacagagggtgatggtgggtgaccttgaggatatcaacgagatgatcatcaagcagaag

SEQ ID NO: 1467 - DNA, 212 bp, synthetic construct
  source 1..212: mol_type = other DNA
ttgaaagaattcagaatcagtgggatgaagtacaagaacaccttcagaaccggaggcaacagttgaatgaaatgttaaaggattcaacacaatggctggaagctaaggaagaagctgagcaggtcttaggacaggccagagccaagcttgagtcatggaaggagggtccctatacagtagatgcaatccaaaagaaaatcacagaaaccaag

SEQ ID NO: 1468 - protein, 748 aa, synthetic construct
PKKKRKVGIHGVPAAMIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAVKRPAATKKAGQAKKKK

SEQ ID NO: 1469 - protein, 928 aa, synthetic construct
MDYKDHDGDYKDHDIDYKDDDDKMAPKKKRKVGIHGVPAAMIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAVKRPAATKKAGQAKKKKEFGSGEGRGSLLTCGDVEENPGPMAKPLSQEESTLIERATATINSIPISEDYSVASAALSSDGRIFTGVNVYHFTGGPCAELVVLGTAAAAAAGNLTCIVAIGNENRGILSPCGRCRQVLLDLHPGIKAIVKDSDGQPTAVGIRELLPSGYVWEG

SEQ ID NO: 1470 - DNA, 60 bp, synthetic construct
  source 1..60: mol_type = other DNA
tcgtcggcagcgtcagatgtgtataagagacagctctgcaaatgcaggaaactatcagag

SEQ ID NO: 1471 - DNA, 54 bp, synthetic construct
  source 1..54: mol_type = other DNA
gtctcgtgggctcggagatgtgtataagagacagtaaaccagctccgtccaggc

SEQ ID NO: 1472 - DNA, 59 bp, synthetic construct
  source 1..59: mol_type = other DNA
tcgtcggcagcgtcagatgtgtataagagacagctgtcttgtatcctttggatatgggc

SEQ ID NO: 1473 - DNA, 62 bp, synthetic construct
  source 1..62: mol_type = other DNA
gtctcgtgggctcggagatgtgtataagagacagctgctgttgattaatggttgataggttc

SEQ ID NO: 1474 - DNA, 63 bp, synthetic construct
  source 1..63: mol_type = other DNA
tcgtcggcagcgtcagatgtgtataagagacaggcaattgataaatatttgtagggtggttgg

SEQ ID NO: 1475 - DNA, 64 bp, synthetic construct
  source 1..64: mol_type = other DNA
gtctcgtgggctcggagatgtgtataagagacagagtcaatttccaaggaatgtactctaagac

SEQ ID NO: 1476 - DNA, 63 bp, synthetic construct
  source 1..63: mol_type = other DNA
tcgtcggcagcgtcagatgtgtataagagacagattgaaattggctctttagcttgtgtttct

SEQ ID NO: 1477 - DNA, 57 bp, synthetic construct
  source 1..57: mol_type = other DNA
gtctcgtgggctcggagatgtgtataagagacagcagttgcctaagaactggtggga

SEQ ID NO: 1478 - DNA, 61 bp, synthetic construct
  source 1..61: mol_type = other DNA
tcgtcggcagcgtcagatgtgtataagagacagttgttcatcatcctagccataacacaat

SEQ ID NO: 1479 - DNA, 62 bp, synthetic construct
  source 1..62: mol_type = other DNA
gtctcgtgggctcggagatgtgtataagagacaggtctactgttcatttcagctttaacgtg

SEQ ID NO: 1480 - protein, 5 aa, synthetic construct
GSGGS

SEQ ID NO: 1481 - protein, 6 aa, synthetic construct
GGSGGS

SEQ ID NO: 1482 - protein, 4 aa, synthetic construct
GGGS

SEQ ID NO: 1483 - protein, 5 aa, synthetic construct
GSGGS

SEQ ID NO: 1484 - protein, 6 aa, synthetic construct
GGSGGS

SEQ ID NO: 1485 - protein, 4 aa, synthetic construct
GGGS

SEQ ID NO: 1486 - protein, 4 aa, synthetic construct
GGSG

SEQ ID NO: 1487 - protein, 5 aa, synthetic construct
GGSGG

SEQ ID NO: 1488 - protein, 5 aa, synthetic construct
GSGSG

SEQ ID NO: 1489 - protein, 5 aa, synthetic construct
GSGGG

SEQ ID NO: 1490 - protein, 5 aa, synthetic construct
GGGSG

SEQ ID NO: 1491 - protein, 5 aa, synthetic construct
GSSSG

SEQ ID NO: 1492 - DNA, 20 bp, synthetic construct
  source 1..20: mol_type = other DNA
  misc_difference 1..20: note = This sequence may encompass 5-20 nucleotides
aaaaaaaaaaaaaaaaaaaa

SEQ ID NO: 1493 - DNA, 20 bp, synthetic construct
  source 1..20: mol_type = other DNA
  misc_difference 1..20: note = This sequence may encompass 5-20 nucleotides
tttttttttttttttttttt

SEQ ID NO: 1494 - DNA, 20 bp, synthetic construct
  source 1..20: mol_type = other DNA
  misc_difference 1..20: note = This sequence may encompass 5-20 nucleotides
cccccccccccccccccccc

SEQ ID NO: 1495 - DNA, 20 bp, synthetic construct
  source 1..20: mol_type = other DNA
  misc_difference 1..20: note = This sequence may encompass 5-20 nucleotides
gggggggggggggggggggg

SEQ ID NO: 1496 - DNA, 40 bp, synthetic construct
  source 1..40: mol_type = other DNA
ctgcccaatgccatcctggagttcctgtaagataccaaaa

SEQ ID NO: 1497 - DNA, 31 bp, synthetic construct
  source 1..31: mol_type = other DNA
ctgcccaatgccatcctgtaagataccaaaa

SEQ ID NO: 1498 - DNA, 16 bp, synthetic construct
  source 1..16: mol_type = other DNA
ctgcccaatgccaaaa

SEQ ID NO: 1499 - DNA, 40 bp, synthetic construct
  source 1..40: mol_type = other DNA
ctgcccaatgccatcctggaggtcctgtaagataccaaaa

SEQ ID NO: 1500 - DNA, 40 bp, synthetic construct
  source 1..40: mol_type = other DNA
ctgcccaatgccatcctggagttcctgtaagataacaaaa

SEQ ID NO: 1501 - DNA, 20 bp, synthetic construct
  source 1..20: mol_type = other DNA
ctgcccaatgccatccaaaa

SEQ ID NO: 1502 - DNA, 40 bp, synthetic construct
  source 1..40: mol_type = other DNA
ctgcccaatgccatcctggagttcctgtaagataccaaac

SEQ ID NO: 1503 - DNA, 30 bp, synthetic construct
  source 1..30: mol_type = other DNA
ctgcccaatgccatctgtaagataccaaaa

SEQ ID NO: 1504 - DNA, 39 bp, synthetic construct
  source 1..39: mol_type = other DNA
ctgcccaatgccatctggagttcctgtaagataccaaaa

SEQ ID NO: 1505 - DNA, 40 bp, synthetic construct
  source 1..40: mol_type = other DNA
ctgcccaatgccatcctggagtgcctgtaagataccaaaa

SEQ ID NO: 1506 - DNA, 39 bp, synthetic construct
  source 1..39: mol_type = other DNA
ctgcccaatgccatcctggagttctgtaagataccaaaa

SEQ ID NO: 1507 - DNA, 36 bp, synthetic construct
  source 1..36: mol_type = other DNA
ctgcccaatgccatcctggactgtaagataccaaaa

SEQ ID NO: 1508 - DNA, 40 bp, synthetic construct
  source 1..40: mol_type = other DNA
ctgcccaatgccatcctggagttactgtaagataccaaaa

SEQ ID NO: 1509 - DNA, 16 bp, synthetic construct
  source 1..16: mol_type = other DNA
ctgcccaataccaaaa

SEQ ID NO: 1510 - DNA, 38 bp, synthetic construct
  source 1..38: mol_type = other DNA
ctgcccaatgccatcctggagttcgtaagataccaaaa

SEQ ID NO: 1511 - DNA, 23 bp, synthetic construct
  source 1..23: mol_type = other DNA
ctgcccaatgtaagataccaaaa

SEQ ID NO: 1512 - DNA, 40 bp, synthetic construct
  source 1..40: mol_type = other DNA
ctgcccaatgccatcctggagttcctgtaagatacaaaaa

SEQ ID NO: 1513 - DNA, 37 bp, synthetic construct
  source 1..37: mol_type = other DNA
ctgcccaatgccatcctggagctgtaagataccaaaa

SEQ ID NO: 1514 - DNA, 40 bp, synthetic construct
  source 1..40: mol_type = other DNA
ctgcccactgccatcctggagttcctgtaagataccaaaa

SEQ ID NO: 1515 - DNA, 40 bp, synthetic construct
  source 1..40: mol_type = other DNA
ctgcccaatgccatcctggagttcctgtaagataccaaat

SEQ ID NO: 1516 - DNA, 40 bp, synthetic construct
  source 1..40: mol_type = other DNA
ctgcccaatgcaatcctggagttcctgtaagataccaaaa

SEQ ID NO: 1517 - DNA, 12 bp, synthetic construct
  source 1..12: mol_type = other DNA
aagataccaaaa

SEQ ID NO: 1518 - DNA, 38 bp, synthetic construct
  source 1..38: mol_type = other DNA
ctgcccaatgccatcggagttcctgtaagataccaaaa

SEQ ID NO: 1519 - DNA, 40 bp, synthetic construct
  source 1..40: mol_type = other DNA
cggcccaatgccatcctggagttcctgtaagataccaaaa

SEQ ID NO: 1520 - DNA, 40 bp, synthetic construct
  source 1..40: mol_type = other DNA
ctgcccaatgccatccttgagttcctgtaagataccaaaa

SEQ ID NO: 1521 - DNA, 19 bp, synthetic construct
  source 1..19: mol_type = other DNA
ctgcccaatgccatcaaaa

SEQ ID NO: 1522 - DNA, 40 bp, synthetic construct
  source 1..40: mol_type = other DNA
ctgcccaatgccatactggagttcctgtaagataccaaaa

SEQ ID NO: 1523 - DNA, 15 bp, synthetic construct
  source 1..15: mol_type = other DNA
ctgcccaatgccatc

SEQ ID NO: 1524 - DNA, 40 bp, synthetic construct
  source 1..40: mol_type = other DNA
ctgcccaatgccttcctggagttcctgtaagataccaaaa

SEQ ID NO: 1525 - DNA, 40 bp, synthetic construct
  source 1..40: mol_type = other DNA
ctgcccaatgccatcctggagttcctgtaagaaaccaaaa

SEQ ID NO: 1526 - DNA, 40 bp, synthetic construct
  source 1..40: mol_type = other DNA
ctgcccaatgccatcctggagatcctgtaagataccaaaa

SEQ ID NO: 1527 - DNA, 40 bp, synthetic construct
  source 1..40: mol_type = other DNA
ctgcccaatgccatcctggagttcctgtacgataccaaaa

SEQ ID NO: 1528 - DNA, 10 bp, synthetic construct
  source 1..10: mol_type = other DNA
ctgcccaatg

SEQ ID NO: 1529 - DNA, 35 bp, synthetic construct
  source 1..35: mol_type = other DNA
ctgcccaatgccatcctggctgtaagataccaaaa

SEQ ID NO: 1530 - DNA, 40 bp, synthetic construct
  source 1..40: mol_type = other DNA
ctgcccaatgccatcctggagttcctgtaagataccaata

SEQ ID NO: 1531 - DNA, 10 bp, synthetic construct
  source 1..10: mol_type = other DNA
ctgcccaaaa

SEQ ID NO: 1532 - DNA, 40 bp, synthetic construct
  source 1..40: mol_type = other DNA
ctgcccaatgccatcctggagttcccgtaagataccaaaa

SEQ ID NO: 1533 - DNA, 16 bp, synthetic construct
  source 1..16: mol_type = other DNA
ctgtaagataccaaaa

SEQ ID NO: 1534 - DNA, 40 bp, synthetic construct
  source 1..40: mol_type = other DNA
ctgcccaatgccatcctggagctcctgtaagataccaaaa

SEQ ID NO: 1535 - DNA, 40 bp, synthetic construct
  source 1..40: mol_type = other DNA
ctgccctatgccatcctggagttcctgtaagataccaaaa

SEQ ID NO: 1536 - DNA, 27 bp, synthetic construct
  source 1..27: mol_type = other DNA
ctgcccaatgcctgtaagataccaaaa

SEQ ID NO: 1537 - DNA, 28 bp, synthetic construct
  source 1..28: mol_type = other DNA
ctgcccaatgccatcctggagttcaaaa

SEQ ID NO: 1538 - DNA, 31 bp, synthetic construct
  source 1..31: mol_type = other DNA
ctgcccaatgccatcctggagtataccaaaa

SEQ ID NO: 1539 - DNA, 40 bp, synthetic construct
  source 1..40: mol_type = other DNA
ctgcccaatgccatcctggagttcctgtaagatcccaaaa

SEQ ID NO: 1540 - DNA, 40 bp, synthetic construct
  source 1..40: mol_type = other DNA
ctgcccaatgccctcctggagttcctgtaagataccaaaa

SEQ ID NO: 1541 - DNA, 40 bp, synthetic construct
  source 1..40: mol_type = other DNA
ctgcacaatgccatcctggagttcctgtaagataccaaaa

SEQ ID NO: 1542 - DNA, 40 bp, synthetic construct
  source 1..40: mol_type = other DNA
ctgcccaatgccatcatggagttcctgtaagataccaaaa

SEQ ID NO: 1543 - DNA, 40 bp, synthetic construct
  source 1..40: mol_type = other DNA
caggttgtgtcaccagagtaacagtctgagtaggagctaa

SEQ ID NO: 1544 - DNA, 25 bp, synthetic construct
  source 1..25: mol_type = other DNA
caggttgtgtctgagtaggagctaa

SEQ ID NO: 1545 - DNA, 40 bp, synthetic construct
  source 1..40: mol_type = other DNA
caggttgtgtccccagagtaacagtctgagtaggagctaa

SEQ ID NO: 1546 - DNA, 28 bp, synthetic construct
  source 1..28: mol_type = other DNA
caggttgtgtcaccagagtaggagctaa

SEQ ID NO: 1547 - DNA, 28 bp, synthetic construct
  source 1..28: mol_type = other DNA
caggttgtgtcacctgagtaggagctaa

SEQ ID NO: 1548 - DNA, 26 bp, synthetic construct
  source 1..26: mol_type = other DNA
caggttgtgtcaccagtaggagctaa

SEQ ID NO: 1549 - DNA, 15 bp, synthetic construct
  source 1..15: mol_type = other DNA
caggttgtgtcacca

SEQ ID NO: 1550 - DNA, 19 bp, synthetic construct
  source 1..19: mol_type = other DNA
caggttgtgtaggagctaa

SEQ ID NO: 1551 - DNA, 13 bp, synthetic construct
  source 1..13: mol_type = other DNA
gagtaggagctaa

SEQ ID NO: 1552 - DNA, 21 bp, synthetic construct
  source 1..21: mol_type = other DNA
caggttgtgagtaggagctaa

SEQ ID NO: 1553 - DNA, 34 bp, synthetic construct
  source 1..34: mol_type = other DNA
caggttgtgtcaccagagtctgagtaggagctaa

SEQ ID NO: 1554 - DNA, 40 bp, synthetic construct
  source 1..40: mol_type = other DNA
caggttgtgtcaccagagtaacaggctgagtaggagctaa

SEQ ID NO: 1555 - DNA, 13 bp, synthetic construct
  source 1..13: mol_type = other DNA
cagtaggagctaa

SEQ ID NO: 1556 - DNA, 12 bp, synthetic construct
  source 1..12: mol_type = other DNA
agtaggagctaa

SEQ ID NO: 1557 - DNA, 19 bp, synthetic construct
  source 1..19: mol_type = other DNA
caggttgagtaggagctaa

SEQ ID NO: 1558 - DNA, 17 bp, synthetic construct
  source 1..17: mol_type = other DNA
caggttgtaggagctaa

SEQ ID NO: 1559 - DNA, 14 bp, synthetic construct
  source 1..14: mol_type = other DNA
tgagtaggagctaa

SEQ ID NO: 1560 - DNA, 27 bp, synthetic construct
  source 1..27: mol_type = other DNA
caggttgtgtcactgagtaggagctaa

SEQ ID NO: 1561 - DNA, 40 bp, synthetic construct
  source 1..40: mol_type = other DNA
cagggtgtgtcaccagagtaacagtctgagtaggagctaa

SEQ ID NO: 1562 - DNA, 32 bp, synthetic construct
  source 1..32: mol_type = other DNA
caggttgtgtcaccagtctgagtaggagctaa

SEQ ID NO: 1563 - DNA, 14 bp, synthetic construct
  source 1..14: mol_type = other DNA
caggtaggagctaa

SEQ ID NO: 1564 - DNA, 40 bp, synthetic construct
  source 1..40: mol_type = other DNA
caggttgtgtcgccagagtaacagtctgagtaggagctaa

SEQ ID NO: 1565 - DNA, 40 bp, synthetic construct
  source 1..40: mol_type = other DNA
caggttgtgtctccagagtaacagtctgagtaggagctaa

SEQ ID NO: 1566 - DNA, 30 bp, synthetic construct
  source 1..30: mol_type = other DNA
caggttgtgtcaccactgagtaggagctaa

SEQ ID NO: 1567 - DNA, 16 bp, synthetic construct
  source 1..16: mol_type = other DNA
tctgagtaggagctaa

SEQ ID NO: 1568 - DNA, 40 bp, synthetic construct
  source 1..40: mol_type = other DNA
caggttgtgtcaccagagtaacagtctgagtatgagctaa

SEQ ID NO: 1569 - DNA, 33 bp, synthetic construct
  source 1..33: mol_type = other DNA
caggttgtgtcaccaagtctgagtaggagctaa

SEQ ID NO: 1570 - DNA, 40 bp, synthetic construct
  source 1..40: mol_type = other DNA
cgggttgtgtcaccagagtaacagtctgagtaggagctaa

SEQ ID NO: 1571 - DNA, 40 bp, synthetic construct
  source 1..40: mol_type = other DNA
caggttgtgtcaccagagtaacagtctgagtaggggctaa

SEQ ID NO: 1572 - DNA, 15 bp, synthetic construct
  source 1..15: mol_type = other DNA
ctgagtaggagctaa

SEQ ID NO: 1573 - DNA, 40 bp, synthetic construct
  source 1..40: mol_type = other DNA
caggttgtgtcaccatagtaacagtctgagtaggagctaa

SEQ ID NO: 1574 - DNA, 10 bp, synthetic construct
  source 1..10: mol_type = other DNA
taggagctaa

SEQ ID NO: 1575 - DNA, 40 bp, synthetic construct
  source 1..40: mol_type = other DNA
caggttgtgccaccagagtaacagtctgagtaggagctaa

SEQ ID NO: 1576 - DNA, 40 bp, synthetic construct
  source 1..40: mol_type = other DNA
caggttgtgtcaccagagtatcagtctgagtaggagctaa

SEQ ID NO: 1577 - DNA, 36 bp, synthetic construct
  source 1..36: mol_type = other DNA
caggttgtgtcaccagagtaactgagtaggagctaa

SEQ ID NO: 1578 - DNA, 20 bp, synthetic construct
  source 1..20: mol_type = other DNA
caggtctgagtaggagctaa

SEQ ID NO: 1579 - DNA, 40 bp, synthetic construct
  source 1..40: mol_type = other DNA
caggttgtgtcacaagagtaacagtctgagtaggagctaa

SEQ ID NO: 1580 - DNA, 10 bp, synthetic construct
  source 1..10: mol_type = other DNA
caggagctaa

SEQ ID NO: 1581 - DNA, 40 bp, synthetic construct
  source 1..40: mol_type = other DNA
caggttgtgtcaccagagtaacagtctgagtaggatctaa

SEQ ID NO: 1582 - DNA, 40 bp, synthetic construct
  source 1..40: mol_type = other DNA
caggttgtgtcaccagagtaacagtctgagtagtagctaa

SEQ ID NO: 1583 - DNA, 40 bp, synthetic construct
  source 1..40: mol_type = other DNA
caggttgtgtcaccagagtaacagtctgagtaggagataa

SEQ ID NO: 1584 - DNA, 40 bp, synthetic construct
  source 1..40: mol_type = other DNA
caggttgtgtcaccagagtaacagtctgaggaggagctaa

SEQ ID NO: 1585 - DNA, 20 bp, synthetic construct
  source 1..20: mol_type = other DNA
caggttgtgtcaccagagta

SEQ ID NO: 1586 - DNA, 22 bp, synthetic construct
  source 1..22: mol_type = other DNA
caggttgtgtcaccagagctaa

SEQ ID NO: 1587 - DNA, 40 bp, synthetic construct
  source 1..40: mol_type = other DNA
agccaaagagaatgggatccagtatacttacaggctccaa

SEQ ID NO: 1588 - DNA, 21 bp, synthetic construct
  source 1..21: mol_type = other DNA
agccaaagagaatggctccaa

SEQ ID NO: 1589 - DNA, 40 bp, synthetic construct
  source 1..40: mol_type = other DNA
agccaaagagaatgggagccagtatacttacaggctccaa

SEQ ID NO: 1590 - DNA, 14 bp, synthetic construct
  source 1..14: mol_type = other DNA
cttacaggctccaa

SEQ ID NO: 1591 - DNA, 40 bp, synthetic construct
  source 1..40: mol_type = other DNA
agccaaagagactgggatccagtatacttacaggctccaa

SEQ ID NO: 1592 - DNA, 21 bp, synthetic construct
  source 1..21: mol_type = other DNA
agccaaacttacaggctccaa

SEQ ID NO: 1593 - DNA, 37 bp, synthetic construct
  source 1..37: mol_type = other DNA
agccaaagagaatgggatccatacttacaggctccaa

SEQ ID NO: 1594 - DNA, 23 bp, synthetic construct
  source 1..23: mol_type = other DNA
agccaaagacttacaggctccaa

SEQ ID NO: 1595 - DNA, 35 bp, synthetic construct
  source 1..35: mol_type = other DNA
agccaaagagaatgggatccacttacaggctccaa

SEQ ID NO: 1596 - DNA, 12 bp, synthetic construct
  source 1..12: mol_type = other DNA
tacaggctccaa

SEQ ID NO: 1597 - DNA, 40 bp, synthetic construct
  source 1..40: mol_type = other DNA
agccaaagagattgggatccagtatacttacaggctccaa

SEQ ID NO: 1598 - DNA, 36 bp, synthetic construct
  source 1..36: mol_type = other DNA
agccaaagagaatgggatccaacttacaggctccaa

SEQ ID NO: 1599 - DNA, 37 bp, synthetic construct
  source 1..37: mol_type = other DNA
agccaaagagaatgggatccagacttacaggctccaa

SEQ ID NO: 1600 - DNA, 22 bp, synthetic construct
  source 1..22: mol_type = other DNA
agccaaagagaatgggatccaa

SEQ ID NO: 1601 - DNA, 20 bp, synthetic construct
  source 1..20: mol_type = other DNA
agccaacttacaggctccaa

SEQ ID NO: 1602 - DNA, 17 bp, synthetic construct
  source 1..17: mol_type = other DNA
agccaaacaggctccaa

SEQ ID NO: 1603 - DNA, 20 bp, synthetic construct
  source 1..20: mol_type = other DNA
agccaaagagaatgctccaa

SEQ ID NO: 1604 - DNA, 40 bp, synthetic construct
  source 1..40: mol_type = other DNA
agacaaagagaatgggatccagtatacttacaggctccaa

SEQ ID NO: 1605 - DNA, 38 bp, synthetic construct
  source 1..38: mol_type = other DNA
agccaaagagaatgggatccagtacttacaggctccaa

SEQ ID NO: 1606 - DNA, 22 bp, synthetic construct
  source 1..22: mol_type = other DNA
agccaaagagaatgggctccaa

SEQ ID NO: 1607 - DNA, 13 bp, synthetic construct
  source 1..13: mol_type = other DNA
agccaggctccaa

SEQ ID NO: 1608 - DNA, 28 bp, synthetic construct
  source 1..28: mol_type = other DNA
agccaaagagaatacttacaggctccaa

SEQ ID NO: 1609 - DNA, 15 bp, synthetic construct
  source 1..15: mol_type = other DNA
agccaaaggctccaa

SEQ ID NO: 1610 - DNA, 16 bp, synthetic construct
  source 1..16: mol_type = other DNA
agcttacaggctccaa

SEQ ID NO: 1611 - DNA, 26 bp, synthetic construct
  source 1..26: mol_type = other DNA
agccaaagagaacttacaggctccaa

SEQ ID NO: 1612 - DNA, 10 bp, synthetic construct
  source 1..10: mol_type = other DNA
caggctccaa

SEQ ID NO: 1613 - DNA, 40 bp, synthetic construct
  source 1..40: mol_type = other DNA
cgccaaagagaatgggatccagtatacttacaggctccaa

SEQ ID NO: 1614 - DNA, 29 bp, synthetic construct
  source 1..29: mol_type = other DNA
agccaaagagaatggcttacaggctccaa

SEQ ID NO: 1615 - DNA, 40 bp, synthetic construct
  source 1..40: mol_type = other DNA
agccaaagagaaggggatccagtatacttacaggctccaa

SEQ ID NO: 1616 - DNA, 15 bp, synthetic construct
  source 1..15: mol_type = other DNA
acttacaggctccaa

SEQ ID NO: 1617 - DNA, 40 bp, synthetic construct
  source 1..40: mol_type = other DNA
agccaacgagaatgggatccagtatacttacaggctccaa

SEQ ID NO: 1618 - DNA, 40 bp, synthetic construct
  source 1..40: mol_type = other DNA
agccaaagagaatgggatccagtatacttacaggcgccaa

SEQ ID NO: 1619 - DNA, 15 bp, synthetic construct
  source 1..15: mol_type = other DNA
agccaaagagaatgg

SEQ ID NO: 1620 - DNA, 11 bp, synthetic construct
  source 1..11: mol_type = other DNA
acaggctccaa

SEQ ID NO: 1621 - DNA, 17 bp, synthetic construct
  source 1..17: mol_type = other DNA
agccaaagaggctccaa

SEQ ID NO: 1622 - DNA, 40 bp, synthetic construct
  source 1..40: mol_type = other DNA
agccaatgagaatgggatccagtatacttacaggctccaa

SEQ ID NO: 1623 - DNA, 40 bp, synthetic construct
  source 1..40: mol_type = other DNA
agccaaagagtatgggatccagtatacttacaggctccaa

SEQ ID NO: 1624 - DNA, 30 bp, synthetic construct
  source 1..30: mol_type = other DNA
agccaaagagaatggacttacaggctccaa

SEQ ID NO: 1625 - DNA, 40 bp, synthetic construct
  source 1..40: mol_type = other DNA
agcaaaagagaatgggatccagtatacttacaggctccaa

SEQ ID NO: 1626 - DNA, 13 bp, synthetic construct
  source 1..13: mol_type = other DNA
agccaaagagaat

SEQ ID NO: 1627 - DNA, 31 bp, synthetic construct
  source 1..31: mol_type = other DNA
agccaaagagaatgggatccacaggctccaa

SEQ ID NO: 1628 - DNA, 13 bp, synthetic construct
  source 1..13: mol_type = other DNA
ttacaggctccaa

SEQ ID NO: 1629 - DNA, 40 bp, synthetic construct
  source 1..40: mol_type = other DNA
agccaaagagaatgggatccagtatacttacaggctccat

SEQ ID NO: 1630 - DNA, 25 bp, synthetic construct
  source 1..25: mol_type = other DNA
agccaaagatacttacaggctccaa

SEQ ID NO: 1631 - DNA, 31 bp, synthetic construct
  source 1..31: mol_type = other DNA
agccaaagagaatgggacttacaggctccaa

SEQ ID NO: 1632 - DNA, 140 bp, synthetic construct
  source 1..140: mol_type = other DNA
accgcttcaccaagtgctgtcccttagggattagcacttgagtgaaggtgggctgcttgcatcagcctaatgtcgagaagtgctttcttcggaaagtaaccctcgaaacaaattcatttgaaagaatgaaggaatgcaac

SEQ ID NO: 1633 - protein, 26 aa, synthetic construct
GSGGSPAGSPTSTEEGTSESATPGSG

SEQ ID NO: 1634 - RNA, 13 nt, synthetic construct
  source 1..13: mol_type = other RNA
tggtacatccaac

SEQ ID NO: 1635 - RNA, 61 nt, synthetic construct
  source 1..61: mol_type = other RNA
tggggcagttggttgcccttagcctgaggcatttattgcactcgggaagtaccatttctca

(19) United States
(12) Patent Application Publication
(10) Pub. No.: US 2024/0384249 A1
(43) Pub. Date: Nov. 21, 2024

(54) SYSTEMS AND USES THEREOF FOR THE TREATMENT OF DMD-ASSOCIATED DISEASES

(71) Applicant: Mammoth Biosciences, Inc., Brisbane, CA (US)

(72) Inventors: Lucas Benjamin HARRINGTON, San Francisco, CA (US); Wiputra Jaya HARTONO, San Francisco, CA (US); Pei-Qi LIU, Oakland, CA (US); Darren Bo Yee LO, Pacifica, CA (US); Renan B. SPER, San Francisco, CA (US)

(21) Appl. No.: 18/679,314

(22) Filed: May 30, 2024

Related U.S. Application Data
(63) Continuation of application No. PCT/US2022/081118, filed on Dec. 7, 2022.
(60) Provisional application No. 63/287,481, filed on Dec. 8, 2021.
(60) Provisional application No. 63/287,489, filed on Dec. 8, 2021.
(60) Provisional application No. 63/287,514, filed on Dec. 8, 2021.
(60) Provisional application No. 63/287,515, filed on Dec. 8, 2021.
(60) Provisional application No. 63/303,439, filed on Jan. 26, 2022.
(60) Provisional application No. 63/303,478, filed on Jan. 26, 2022.
(60) Provisional application No. 63/303,489, filed on Jan. 26, 2022.
(60) Provisional application No. 63/303,495, filed on Jan. 26, 2022.
(60) Provisional application No. 63/334,638, filed on Apr. 25, 2022.
(60) Provisional application No. 63/334,641, filed on Apr. 25, 2022.
(60) Provisional application No. 63/334,645, filed on Apr. 25, 2022.
(60) Provisional application No. 63/334,657, filed on Apr. 25, 2022.
(60) Provisional application No. 63/346,499, filed on May 27, 2022.
(60) Provisional application No. 63/346,527, filed on May 27, 2022.
(60) Provisional application No. 63/346,537, filed on May 27, 2022.
(60) Provisional application No. 63/346,550, filed on May 27, 2022.
(60) Provisional application No. 63/355,989, filed on Jun. 27, 2022.

(51) Int. Cl. C12N 9/22; A61K 31/7088; A61K 38/46; C12N 15/11; C12N 15/86

(52) U.S. Cl. CPC C12N 9/22; A61K 31/7088; A61K 38/465; C12N 15/11; C12N 15/86; C07K 2319/00; C12N 2310/20; C12N 2750/14143; C12N 2750/14171

(57) ABSTRACT

Provided herein are compositions, systems, and methods comprising effector proteins and uses thereof. These effector proteins may be characterized as CRISPR-associated (Cas) proteins. Various compositions, systems, and methods of the present disclosure may leverage the activities of these effector proteins for the modification, detection, and engineering of nucleic acids, including editing target nucleic acids, as well as, the treatment of diseases and disorders associated with the dystrophin gene (DMD).

### CROSS-REFERENCED APPLICATIONS

This application is a continuation of International Patent Application No. PCT/US2022/081118, filed Dec. 7, 2022, which claims the benefit of priority of U.S. Provisional Application No. 63/287,481, filed on Dec. 8, 2021, U.S. Provisional Application No. 63/287,489, filed on Dec. 8, 2021, U.S. Provisional Application No. 63/287,514, filed on Dec. 8, 2021, U.S. Provisional Application No. 63/287,515, filed on Dec. 8, 2021, U.S. Provisional Application No. 63/303,439, filed on Jan. 26, 2022, U.S. Provisional Application No. 63/303,495, filed on Jan. 26, 2022, U.S. Provisional Application No. 63/303,489, filed on Jan. 26, 2022, U.S. Provisional Application No. 63/303,478, filed on Jan. 26, 2022, U.S. Provisional Application No. 63/334,641, filed on Apr. 25, 2022, U.S. Provisional Application No. 63/334,638, filed on Apr. 25, 2022, U.S. Provisional Application No. 63/334,657, filed on Apr. 25, 2022, U.S. Provisional Application No. 63/334,645, filed on Apr. 25, 2022, U.S. Provisional Application No. 63/346,499, filed on May 27, 2022, U.S. Provisional Application No. 63/346,527, filed on May 27, 2022, U.S. Provisional Application No. 63/346,550, filed on May 27, 2022, U.S. Provisional Application No. 63/346,537, filed on May 27, 2022 and U.S. Provisional Application No. 63/355,989, filed on Jun. 27, 2022, the entire contents of each of which are incorporated herein by reference.

### INCORPORATION BY REFERENCE OF SEQUENCE LISTING

The instant application contains a Sequence Listing, which has been submitted via Patent Center. The Sequence Listing titled 203477-717301_US_SL.xml, which was created on May 23, 2024 and is 1,499,929 bytes in size, is hereby incorporated by reference in its entirety.

### FIELD

The present disclosure relates generally to compositions of effector proteins and guide nucleic acids, and methods and systems of using such compositions, including detecting and editing target nucleic acids, as well as, the treatment of diseases and disorders associated with the dystrophin gene (DMD).

### BACKGROUND

Clustered Regularly Interspaced Short Palindromic Repeats (CRISPR) and associated proteins (Cas proteins), sometimes referred to as a CRISPR/Cas system, were first identified in certain bacterial species and are now understood to form part of a prokaryotic acquired immune system. CRISPR/Cas systems provide immunity in bacteria and archaea against viruses and plasmids by targeting the nucleic acids of the viruses and plasmids in a sequence-specific manner. While CRISPR/Cas proteins are involved in the acquisition, targeting and cleavage of foreign DNA or RNA, the systems may also contain a CRISPR array, which includes direct repeats flanking short spacer sequences that, in part, guide Cas proteins to their targets. The discovery of CRISPR/Cas systems has revolutionized the field of genomic manipulation and engineering. Yet, the discovery suffers from several shortcomings that restricts its use for basic biomedical research and therapeutic applications. In particular, compositions and methods for selective targeting and precise nucleobase editing, and/or precise introduction of donor nucleic acids into genomic loci that can help to treat a genetic disease still need to be developed. While the programmable nature of these systems has promising implications in the field of genome engineering, there remains a need to explore alternative strategies and components to leverage the CRISPR-Cas system in ways that are efficient for in vitro detection and effective for in vivo genome engineering. Effector proteins, guide nucleic acids, compositions, systems and methods described herein satisfy this need and provides related advantages.

### SUMMARY

The present disclosure provides for compositions and systems comprising an effector protein, a guide nucleic acid, and uses thereof. Compositions, systems, and methods disclosed herein leverage nucleic acid modifying activities (e.g., cis cleavage activity) of these effector proteins and guide nucleic acids for the modification and detection of target nucleic acids of the DMD gene. Accordingly, in one aspect, provided herein is a composition comprising an effector protein and a guide nucleic acid for the treatment of a disease or disorder associated with the DMD gene.

### CERTAIN EMBODIMENTS

Provided herein in a composition that comprises: (a) an effector protein or a nucleic acid encoding the effector protein, and (b) a guide nucleic acid or a nucleic acid encoding the guide nucleic acid, wherein the guide nucleic acid comprises a spacer sequence that hybridizes to a target sequence in a target nucleic acid and is at least 90% identical to any one of the nucleotide sequences recited in TABLE 4. In some embodiments, the spacer sequence is at least 95% identical to any one of the nucleotide sequences in TABLE 4. In some embodiments, the spacer sequence comprises any one of the nucleotide sequences recited in TABLE 4. In some embodiments, the guide nucleic acid comprises a nucleotide sequence that is at least 90% identical to any one of the nucleotide sequences recited in TABLE 7. In some embodiments, the guide nucleic acid comprises a nucleotide sequence that is at least 95% identical to any one of the nucleotide sequences recited in TABLE 7. In some embodiments, the guide nucleic acid comprises any one of the nucleotide sequences recited in TABLE 7. In some embodiments, the guide nucleic acid comprises a nucleotide sequence that is at least 90% identical to any one of the nucleotide sequences recited in TABLE 8. In some embodiments, the guide nucleic acid is at least 95% identical to any one of the nucleotide sequences recited in TABLE 8. In some embodiments, the guide nucleic acid comprises any one of the sgRNA sequences recited in TABLE 8. In some embodiments, the guide nucleic acid comprises a nucleotide sequence that interacts with the effector protein and is at least 90% identical to any one of the nucleotide sequences recited in TABLE 5. TABLE 5.1 and TABLE 6. In some embodiments, the nucleotide sequence is at least 95% identical any one of the nucleotide sequences set forth in TABLE 5. TABLE 5.1 and TABLE 6. In some embodiments, the nucleotide sequence comprises any one of the nucleotide sequences set forth in TABLE 5. TABLE 5.1 and TABLE 6. In some embodiments, the effector protein comprises an amino acid sequence that is at least 80%, at least 85%, at least 90%, at least 95%, at least 98%, or 100% identical to any one of the amino acid sequences recited in TABLE 1.

In some embodiments. (a) the spacer sequence is at least 90%, at least 95%, or 100% identical to any one of the nucleotide sequences recited in SEQ ID NOS: 28-111 of TABLE 4, optionally wherein the spacer sequence is at least 90%, at least 95%, or 100% identical to any one of the nucleotide sequences recited in SEQ ID NOS: 28, 30, 31, 40, 42, 43, 54, 55, 58, 62, 64, 65, 68, 69, and 79; (b) the guide nucleic acid comprises a nucleotide sequence that interacts with the effector protein and is at least 90%, at least 95%, or 100% identical to the sequence recited in SEQ ID NO: 739 of TABLE 5; (c) the effector protein comprises an amino acid sequence that is at least 80%, at least 85%, at least 90%, at least 95%, at least 98%, or 100% identical to SEQ ID NO: 1 of TABLE 1; or (d) any combination of (a)-(c).

In some embodiments. (a) the spacer sequence is at least 90%, at least 95%, or 100% identical to any one of the nucleotide sequences recited in SEQ ID NOS: 112-621 of TABLE 4, optionally wherein the spacer sequence is at least 90%, at least 95%, or 100% identical to any one of the nucleotide sequences recited in SEQ ID NOS: 113, 114, 118, 119, 144, 145, 146, 150, 156, 158, 160, 162, 166, 170, 171, 175, 176, 178, 179, 182, 183, 236, 304, 305, 317, 337, 340, 398, 405, 412, 425, 453, 455, and 537; (b) the guide nucleic acid comprises a nucleotide sequence that interacts with the effector protein and is at least 90%, at least 95%, or 100% identical to any one of the nucleotide sequences recited in SEQ ID NOS: 740-742 of TABLE 5; (c) the effector protein comprises an amino acid sequence that is at least 80%, at least 85%, at least 90%, at least 95%, at least 98%, or 100% identical to SEQ ID NO: 2 of TABLE 1 or wherein the effector protein comprises an alteration set forth in TABLE 1.1, and other than the alteration set forth in TABLE 1.1, comprises an amino acid sequence that is at least 80%, at least 85%, at least 90%, at least 95%, at least 98%, or at least 99% identical to SEQ ID NO: 2 of TABLE 1, optionally wherein the alteration is an L26R alteration; or (d) any combination of (a)-(c).

In some embodiments. (a) the spacer sequence is at least 90%, at least 95%, or 100% identical to any one of the nucleotide sequences recited in SEQ ID NOS: 622-627 of TABLE 4, optionally wherein the spacer sequence is at least 90%, at least 95%, or 100% identical to any one of the nucleotide sequences recited in SEQ ID NOS: 622, 623 and 626; (b) the guide nucleic acid comprises a nucleotide sequence that interacts with the effector protein and is at least 90%, at least 95%, or 100% identical to any one of the nucleotide sequences recited in SEQ ID NO: 1634 of TABLE 5. SEQ ID NO: 1635 of TABLE 5.1, and SEQ ID NO: 743 of TABLE 6; (c) the effector protein comprises an amino acid sequence that is at least 80%, at least 85%, at least 90%, at least 95%, at least 98%, or 100% identical to SEQ ID NO: 3; or (d) any combination of (a)-(c).

In some embodiments. (a) the spacer sequence is at least 90%, at least 95%, or 100% identical to any one of the nucleotide sequences recited in SEQ ID NOS: 628-738 of TABLE 4, optionally wherein the spacer sequence is at least 90%, at least 95%, or 100% identical to any one of the nucleotide sequences recited in SEQ ID NOS: 634, 638, 646, 659, 660, 661, 685, 686, 687, 694, 703, 704, 710, 713, and 721; (b) the guide nucleic acid comprises a nucleotide sequence that interacts with the effector protein and is at least 90%, at least 95%, or 100% identical to any one of the nucleotide sequences recited in SEQ ID NO: 745 of TABLE 5. SEQ ID NO: 747 of TABLE 5.1, and SEQ ID NOS: 744-746 of TABLE 6; (c) the effector protein comprises an amino acid sequence that is at least 80%, at least 85%, at least 90%, at least 95%, at least 98%, or 100% identical to SEQ ID NO: 4; or (d) any combination of (a)-(c).

In some embodiments, the guide nucleic acid is a crRNA. In some embodiments, the guide nucleic acid is a sgRNA. In some embodiments, the guide nucleic acid comprises one or more phosphorothioate (PS) backbone modifications, 2′-fluoro (2′-F) sugar modifications, or 2′-O-Methyl (2′OMe) sugar modifications. In some embodiments, the guide nucleic acid comprises a spacer sequence and wherein the spacer sequence comprises at least 10 contiguous nucleotides that are reverse complementary to the target sequence. In some embodiments, the target nucleic acid is any one of the nucleic acids set forth in TABLE 9, 9.1, and 9.3. In some embodiments, the target sequence is adjacent to a protospacer adjacent motif (PAM). In some embodiments, the effector protein recognizes a PAM sequence recited in TABLE 3. In some embodiments, the effector protein is a nuclease that is capable of cleaving at least one strand of a target nucleic acid. In some embodiments, the effector protein is fused to a nuclear localization signal. In some embodiments, the effector protein comprises at least one mutation that reduces its nuclease activity, relative to an otherwise comparable effector protein without the mutation, as measured in a cleavage assay. In some embodiments, a fusion partner is fused to the effector protein, or the composition comprises a nucleic acid encoding a fusion partner fused to the effector protein. In some embodiments, the fusion partner is directly fused or linked to the N terminus or C terminus of the effector protein. In some embodiments, the fusion partner is selected from a reverse transcriptase, a deaminase, a transcriptional activator, a transcriptional repressor, or a functional domain thereof. In some embodiments, compositions provided herein comprise a donor nucleic acid. In some embodiments, the target nucleic acid is any one of the nucleic acids set forth in TABLE 9, 9.1, and 9.3, the disease or disorder is any one of the diseases or disorders set forth in TABLE 10, or both. In some embodiments, compositions provided herein comprise a reverse transcriptase and a template RNA. In some embodiments, the reverse transcriptase is covalently linked to the effector protein, the template RNA is covalently linked to the guide nucleic acid, or a combination thereof. In some embodiments, the template RNA is linked to the 5′ end of the guide nucleic acid.

Also provided herein is a nucleic acid expression vector that encodes a guide nucleic acid that comprises a spacer sequence that hybridizes to a target sequence in a target nucleic acid and is at least 90% identical to any one of the nucleotide sequences recited in TABLE 4. In some embodiments, at least one nucleic acid expression vector is a viral vector. In some embodiments, the viral vector is an adeno associated viral (AAV) vector. In some embodiments, the viral vector comprises a nucleotide sequence of a first promoter, wherein the first promoter drives transcription of a nucleotide sequence encoding the guide nucleic acid, and wherein the first promoter is selected from a group consisting of CMV, EF1a, SV40, PGK1, Ubc, human beta actin, CAG, TRE, UAS, Ac5, polyhedron, CaMKIIa, GAL1-10, TEF1, GDS, ADH1, CaMV35S, Ubi, H1, U6, CaMV35S, SV40, CMV, 7SK, and HSV TK. In some embodiments, the viral vector comprises a nucleic acid sequence encoding an effector protein, and wherein an amino acid sequence of the effector protein is at least 80% identical to any one of sequences recited in TABLE 1.

Also provided herein are pharmaceutical compositions, comprising any one of the compositions described herein, or any nucleic acid expression vector described herein; and a pharmaceutically acceptable excipient, carrier or diluent.

Also provided herein are systems comprising components comprising any one of the compositions described herein, one or more components any one of the compositions described herein, or any nucleic acid expression vector described herein. In some embodiments, systems provided here comprise one or more of: (a) at least one detection reagent for detecting a target nucleic acid, optionally wherein: (i) the at least one detection reagent is selected from a reporter nucleic acid, a detection moiety, an additional effector protein, or a combination thereof, optionally wherein the reporter nucleic acid comprises a fluorophore, a quencher, or a combination thereof; and/or (ii) wherein the detection reagent is operably linked to the effector protein or the guide nucleic acid, such that a detection event occurs upon contacting the system with a target nucleic acid; or (b) at least one amplification reagent for amplifying a target nucleic acid, optionally wherein the at least one amplification reagent is selected from the group consisting of a primer, an activator, a dNTP, an rNTP, and combinations thereof.

Also provided herein are methods of modifying a target nucleic acid within a human dystrophin gene, or associated with expression of a human dystrophin gene, the method comprising contacting the target nucleic acid with any one of the compositions described herein, any nucleic acid expression vector described herein, any one of the pharmaceutical compositions described herein, or any one of the systems described herein, thereby modifying the target nucleic acid. In some embodiments, the target nucleic acid comprises a mutation associated with a disease or disorder. In some embodiments, the method is performed in a cell. In some embodiments, the method is performed in vivo. In some embodiments, the target nucleic acid is any one of the nucleic acids set forth in TABLE 9, 9.1, and 9.3. In some embodiments, the target nucleic acid is the DMD gene.

Also provided herein is a cell contacted by any one of the compositions described herein, any nucleic acid expression vector described herein, any pharmaceutical composition described herein, any one of the systems described herein, or any method described herein. Also provided herein is a cell comprising any one of the compositions described herein, any nucleic acid expression vector described herein, any pharmaceutical composition described herein, or any one of the systems described herein. Also provided herein is a cell that comprises a target nucleic acid modified by any one of the compositions described herein, any nucleic acid expression vector described herein, any pharmaceutical composition described herein, any one of the systems described herein, or any method described herein. In some embodiments, the cell is a human cell, optionally wherein the cell is a muscle cell or a stem cell. In some embodiments, the cell is a: cardiac muscle cell, a cardiomyocyte, a myocyte, a smooth muscle cell, a skeletal muscle cell, or a visceral muscle cell. In some embodiments, the cell is a: muscle satellite cell, muscle stem cell, myoblast, muscle progenitor cell, induced pluripotent stem cell (iPSC) or a cell derived from an iPSC.

Also provided herein is a population of cells that comprises at least one cell of one of the cells described herein.

Also provided herein are methods of treating a disease associated with a mutation of a human dystrophin gene in a subject in need thereof, the method comprising administering to the subject: (a) any one of the compositions described herein; (b) any nucleic acid expression vector described herein: (c) any pharmaceutical composition described herein; or (d) any one of the systems described herein. In some embodiments, the disease or disorder is any one of the diseases or disorders set forth in TABLE 10. In some embodiments, the disease or disorder is Duchenne muscular dystrophy (DMD), becker muscular dystrophy (BMD), or x-linked dilated cardiomyopathy (CMD) Type 3B. In some embodiments, the disease is DMD.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 illustrates an exemplary schematic of AAV construct for gene editing according to one or more embodiments of the present disclosure. Included in FIG. 1 are the following abbreviations representing elements of the AAV construct: ITR=Inverted terminal repeat; gRNA=guide RNA; UTR=untranslated region; ssAAV=single-stranded AAV; scAAV=self-complementary AAV; and WPRE=Woodchuck Hepatitis Virus (WHV) posttranscriptional regulatory element.

FIG. 2 illustrates exemplary schematics of ssAAV constructs for gene editing according to one or more embodiments of the present disclosure. Included in FIG. 2 are the following abbreviations representing elements of the AAV construct: ITR=Inverted terminal repeat; gRNA=guide RNA; UTR=untranslated region; WPRE=Woodchuck Hepatitis Virus (WHV) posttranscriptional regulatory element; and hGH Poly A=human growth hormone polyadenylation signal.

FIG. 3 illustrates exemplary schematics of ssAAV constructs for gene editing according to one or more embodiments of the present disclosure. Included in FIG. 3 are the following abbreviations representing elements of the AAV construct: ITR=Inverted terminal repeat; gRNA=guide RNA; UTR=untranslated region; WPRE=Woodchuck Hepatitis Virus (WHV) posttranscriptional regulatory element; and hGH Poly A=human growth hormone polyadenylation signal.

FIG. 4 shows indel activity of the effector protein against each targeted exon of interest.

FIG. 5 is an exemplary raw sequencing data for a sample of data obtained from Example 10 with guides targeting the 5′ region of a targeted exon of interest. FIG. 5 discloses SEQ ID NOS: 1496 and 1496-1542, respectively, in order of appearance.

FIG. 6 is an exemplary raw sequencing data for a sample of data obtained from Example 10 with guides targeting a premature stop codon in a targeted exon of interest. FIG. 6 discloses SEQ ID NOS 1543 and 1543-1586, respectively, in order of appearance.

FIG. 7 is an exemplary raw sequencing data for a sample of data obtained from Example 10 with guides targeting the 3′ region of a targeted exon of interest. FIG. 7 discloses SEQ ID NOS 1587 and 1587-1631, respectively, in order of appearance.

FIG. 8A shows cardiomyocyte viability after plasmid delivery by lipofection.

FIG. 8B shows cardiomyocyte viability after mRNA delivery by lipofection.

FIG. 8C shows GFP integration of cardiomyocytes 48 hours after plasmid delivery by lipofection.

FIG. 8D shows GFP integration of cardiomyocytes 48 hours after mRNA delivery by lipofection.

FIG. 8E shows GFP luminescence intensity of cardiomyocytes 48 hours after plasmid delivery by lipofection.

FIG. 8F shows GFP luminescence intensity of cardiomyocytes 48 hours after mRNA delivery by lipofection.

FIG. 9A depicts DMD gene editing in cardiomyocytes.

FIG. 9B depicts DMD gene editing in cardiomyocytes.

FIG. 10A shows myoblast viability after plasmid delivery by lipofection.

FIG. 10B shows myoblast viability after mRNA delivery by lipofection.

FIG. 10C shows GFP integration in myoblasts 48 hours after plasmid delivery by lipofection.

FIG. 10D shows GFP integration in myoblasts 48 hours after mRNA delivery by lipofection.

FIG. 10E shows GFP luminescence intensity of myoblast 48 hours after plasmid delivery by lipofection.

FIG. 10F shows GFP luminescence intensity of myoblast 48 hours after mRNA delivery by lipofection.

FIG. 11 depicts DMD gene editing in myoblasts.

FIG. 12 shows CasPhi.12 mediated modification of human DMD in primary skeletal myoblasts.

### DETAILED DESCRIPTION OF THE INVENTION

It is to be understood that both the foregoing general description and the following detailed description are exemplary, and explanatory only, and are not restrictive of the disclosure.

The section headings used herein are for organizational purposes only and are not to be construed as limiting the subject matter described.

All documents, or portions of documents, cited in this application, including, but not limited to, patents, patent applications, articles, books, and treatises, are hereby expressly incorporated by reference in their entirety for any purpose.

### Definitions

The terms “% identical,” “% identity,” and “percent identity,” or grammatical equivalents thereof, refer to the extent to which two sequences (nucleotide or amino acid) have the same residue at the same positions in an alignment. For example, “an amino acid sequence is X % identical to SEQ ID NO: Y” can refer to % identity of the amino acid sequence to SEQ ID NO: Y and is elaborated as X % of residues in the amino acid sequence are identical to the residues of sequence disclosed in SEQ ID NO: Y. Generally, computer programs can be employed for such calculations. Illustrative programs that compare and align pairs of sequences, include ALIGN (Myers and Miller. Comput Appl Biosci. 1988 March; 4 (1): 11-7), FASTA (Pearson and Lipman, Proc Natl Acad Sci USA. 1988 April; 85 (8): 2444-8; Pearson, Methods Enzymol. 1990; 183:63-98) and gapped BLAST (Altschul et al., Nucleic Acids Res. 1997 Sep. 1; 25 (17); 3389-40), BLASTP, BLASTN, or GCG (Devereux et al., Nucleic Acids Res. 1984 Jan. 11; 12 (1 Pt 1): 387-95).

The terms “amplification” and “amplifying.” or grammatical equivalents thereof, as used herein, refers to a process by which a nucleic acid molecule is enzymatically copied to generate a plurality of nucleic acid molecules containing the same sequence as the original nucleic acid molecule or a distinguishable portion thereof.

The term “base editing enzyme.” as used herein, refers to a protein, polypeptide or fragment thereof that is capable of catalyzing the chemical modification of a nucleobase of a deoxyribonucleotide or a ribonucleotide. Such a base editing enzyme, for example, is capable of catalyzing a reaction that modifies a nucleobase that is present in a nucleic acid molecule, such as DNA or RNA (single stranded or double stranded). Non-limiting examples of the type of modification that a base editing enzyme is capable of catalyzing includes converting an existing nucleobase to a different nucleobase, such as converting a cytosine to a guanine or thymine or converting an adenine to a guanine, hydrolytic deamination of an adenine or adenosine, or methylation of cytosine (e.g., CpG, CpA, CpT or CpC). A base editing enzyme itself may or may not bind to the nucleic acid molecule containing the nucleobase.

The term “base editor,” as used herein, refers to a fusion protein comprising a base editing enzyme fused to an effector protein. The base editor is functional when the effector protein is coupled to a guide nucleic acid. The guide nucleic acid imparts sequence specific activity to the base editor. By way of non-limiting example, the effector protein may comprise a catalytically inactive effector protein. Also, by way of non-limiting example, the base editing enzyme may comprise deaminase activity. Additional base editors are described herein.

The term “catalytically inactive effector protein,” as used herein, refers to an effector protein that is modified relative to a naturally-occurring effector protein to have a reduced or eliminated catalytic activity relative to that of the naturally-occurring effector protein, but retains its ability to interact with a guide nucleic acid. The catalytic activity that is reduced or eliminated is often a nuclease activity. The naturally-occurring effector protein may be a wildtype protein. In some embodiments, the catalytically inactive effector protein is referred to as a catalytically inactive variant of an effector protein, e.g., a Cas effector protein.

The term “cis cleavage,” as used herein, refers to cleavage (hydrolysis of a phosphodiester bond) of a target nucleic acid by an effector protein complexed with a guide nucleic acid refers to cleavage of a target nucleic acid that is hybridized to a guide nucleic acid, wherein cleavage occurs within or directly adjacent to the region of the target nucleic acid that is hybridized to the guide nucleic acid.

The terms “complementary” and “complementarity.” as used herein, with reference to a nucleic acid molecule or nucleotide sequence, refer to the characteristic of a polynucleotide having nucleotides that base pair with their Watson-Crick counterparts (C with G; or A with T) in a reference nucleic acid. For example, when every nucleotide in a polynucleotide forms a base pair with a reference nucleic acid, that polynucleotide is said to be 100% complementary to the reference nucleic acid. In a double stranded DNA or RNA sequence, the upper (sense) strand sequence is in general, understood as going in the direction from its 5′- to 3′-end, and the complementary sequence is thus understood as the sequence of the lower (antisense) strand in the same direction as the upper strand. Following the same logic, the reverse sequence is understood as the sequence of the upper strand in the direction from its 3′- to its 5′-end, while the ‘reverse complement’ sequence or the ‘reverse complementary’ sequence is understood as the sequence of the lower strand in the direction of its 5′- to its 3′-end. Each nucleotide in a double stranded DNA or RNA molecule that is paired with its Watson-Crick counterpart called its complementary nucleotide.

The term “cleavage assay,” as used herein, refers to an assay designed to visualize, quantitate or identify cleavage of a nucleic acid. In some cases, the cleavage activity may be cis-cleavage activity. In some cases, the cleavage activity may be trans-cleavage activity.

The terms “cleave.” “cleaving.” and “cleavage,” as used herein, with reference to a nucleic acid molecule or nuclease activity of an effector protein, refer to the hydrolysis of a phosphodiester bond of a nucleic acid molecule that results in breakage of that bond. The result of this breakage can be a nick (hydrolysis of a single phosphodiester bond on one side of a double-stranded molecule), single strand break (hydrolysis of a single phosphodiester bond on a single-stranded molecule) or double strand break (hydrolysis of two phosphodiester bonds on both sides of a double-stranded molecule) depending upon whether the nucleic acid molecule is single-stranded (e.g., ssDNA or ssRNA) or double-stranded (e.g., dsDNA) and the type of nuclease activity being catalyzed by the effector protein.

The term “clustered regularly interspaced short palindromic repeats (CRISPR),” as used herein, refers to a segment of DNA found in the genomes of certain prokaryotic organisms, including some bacteria and archaea, that includes repeated short sequences of nucleotides interspersed at regular intervals between unique sequences of nucleotides derived from the DNA of a pathogen (e.g., virus) that had previously infected the organism and that functions to protect the organism against future infections by the same pathogen.

The terms “CRISPR RNA” or “crRNA,” as used herein, refer to a type of guide nucleic acid, wherein the nucleic acid is RNA comprising a first sequence, often referred to herein as a spacer sequence, that hybridizes to a target sequence of a target nucleic acid, and a second sequence that either a) hybridizes to a portion of a tracrRNA or b) is capable of being non-covalently bound by an effector protein. In some instances, the second sequence is referred to as a repeat sequence.

The term “detectable signal,” as used herein, refers to a signal that can be detected using optical, fluorescent, chemiluminescent, electrochemical and other detection methods known in the art.

The term “donor nucleic acid,” as used herein, refers to a nucleic acid that is incorporated into a target nucleic acid or target sequence.

The term “donor nucleotide,” as used herein, refers to a single nucleotide that is incorporated into a target nucleic acid. A nucleotide is typically inserted at a site of cleavage by an effector protein.

The term “effector protein,” as used herein, refers to a protein, polypeptide, or peptide that non-covalently binds to a guide nucleic acid to form a complex that contacts a target nucleic acid, wherein at least a portion of the guide nucleic acid hybridizes to a target sequence of the target nucleic acid. A complex between an effector protein and a guide nucleic acid can include multiple effector proteins or a single effector protein. In some instances, the effector protein modifies the target nucleic acid when the complex contacts the target nucleic acid. In some instances, the effector protein does not modify the target nucleic acid, but it is fused to a fusion partner protein that modifies the target nucleic acid when the complex contacts the target nucleic acid. A non-limiting example of an effector protein modifying a target nucleic acid is cleaving of a phosphodiester bond of the target nucleic acid. Additional examples of modifications an effector protein can make to target nucleic acids are described herein and throughout.

The term “functional domain,” as used herein, refers to a region of one or more amino acids in a protein that is required for an activity of the protein, or the full extent of that activity, as measured in an in vitro assay. Activities include, but are not limited to nucleic acid binding, nucleic acid modification, nucleic acid cleavage, protein binding. The absence of the functional domain, including mutations of the functional domain, would abolish or reduce activity.

The term “functional fragment,” as used herein, refers to a fragment of a protein that retains some function relative to the entire protein. Non-limiting examples of functions are nucleic acid binding, protein binding, nuclease activity, nickase activity, deaminase activity, demethylase activity, or acctylation activity.

The terms “fusion effector protein.” “fusion protein,” and “fusion polypeptide.” as used herein, refer to a protein comprising at least two heterologous polypeptides. Often a fusion effector protein comprises an effector protein and a fusion partner protein. In general, the fusion partner protein is not an effector protein. Examples of fusion partner proteins are provided herein.

The terms “fusion partner protein” or “fusion partner.” as used herein, refer to a protein, polypeptide or peptide that is fused to an effector protein. The fusion partner generally imparts some function to the fusion protein that is not provided by the effector protein. The fusion partner may provide a detectable signal. The fusion partner may modify a target nucleic acid, including changing a nucleobase of the target nucleic acid and making a chemical modification to one or more nucleotides of the target nucleic acid. The fusion partner may be capable of modulating the expression of a target nucleic acid. The fusion partner may inhibit, reduce, activate or increase expression of a target nucleic acid via additional proteins or nucleic acid modifications to the target sequence.

A “genetic disease”, as used herein, refers to a disease, disorder, condition, or syndrome caused by one or more mutations in the DNA of an organism. Mutations can be due to several different cellular mechanisms, including, but not limited to, an error in DNA replication, recombination, or repair, or due to environmental factors. Mutations may be encoded in the sequence of a target nucleic acid from the germline of an organism. A genetic disease comprises, in some embodiments, a single gene disorder, a chromosome disorder, or a multifactorial disorder. Said another way, a genetic disease comprises, in some embodiments, a single mutation, multiple mutations, or a chromosomal aberration.

The term “guide nucleic acid.” as used herein, refers to a nucleic acid comprising: a first nucleotide sequence that hybridizes to a target nucleic acid; and a second nucleotide sequence that is capable of being non-covalently bound by an effector protein. The first sequence may be referred to herein as a spacer sequence. The second sequence may be referred to herein as a repeat sequence. In some instances, the first sequence is located 5′ of the second nucleotide sequence. In some instances, the first sequence is located 3′ of the second nucleotide sequence.

The term “heterologous.” as used herein, means a nucleotide or polypeptide sequence that is not found in a native nucleic acid or protein, respectively. In some embodiments, fusion proteins comprise an effector protein and a fusion partner protein, wherein the fusion partner protein is heterologous to an effector protein. These fusion proteins may be referred to as a “heterologous protein.” A protein that is heterologous to the effector protein is a protein that is not covalently linked via an amide bond to the effector protein in nature. In some embodiments, a heterologous protein is not encoded by a species that encodes the effector protein. In some instances, the heterologous protein exhibits an activity (e.g., enzymatic activity) when it is fused to the effector protein. In some instances, the heterologous protein exhibits increased or reduced activity (e.g., enzymatic activity) when it is fused to the effector protein, relative to when it is not fused to the effector protein. In some instances, the heterologous protein exhibits an activity (e.g., enzymatic activity) that it does not exhibit when it is fused to the effector protein. A guide nucleic acid may comprise a first sequence and a second sequence, wherein the first sequence and the second sequence are not found covalently linked via a phosphodiester bond in nature. Thus, the first sequence is considered to be heterologous with the second sequence, and the guide nucleic acid may be referred to as a heterologous guide nucleic acid.

The term, “in vitro.” as used herein, is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed. The term “in vivo” is used to describe an event that takes place in a subject's body. The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “linked amino acids” as used herein, refers to at least two amino acids linked by an amide bond.

The term “linker,” as used herein, refers to a bond or molecule that links a first polypeptide to a second polypeptide or a first nucleic acid to a second nucleic acid. A “peptide linker” comprises at least two amino acids linked by an amide bond.

The term “modified target nucleic acid,” as used herein, refers to a target nucleic acid, wherein the target nucleic acid has undergone a modification, for example, after contact with an effector protein. In some cases, the modification is an alteration in the sequence of the target nucleic acid. In some cases, the modified target nucleic acid comprises an insertion, deletion, or replacement of one or more nucleotides compared to the unmodified target nucleic acid.

The term “mutation associated with a disease,” as used herein, refers to the co-occurrence of a mutation and the phenotype of a disease. The mutation may occur in a gene, wherein transcription or translation products from the gene occur at a significantly abnormal level or in an abnormal form in a cell or subject harboring the mutation as compared to a non-disease control subject not having the mutation.

The terms “non-naturally occurring” and “engineered,” as used herein, are used interchangeably and indicate the involvement of the hand of man. The terms, when referring to a nucleic acid, nucleotide, protein, polypeptide, peptide or amino acid, refer to a nucleic acid, nucleotide, protein, polypeptide, peptide or amino acid that is at least substantially free from at least one other feature with which it is naturally associated in nature and as found in nature, and/or contains a modification (e.g., chemical modification, nucleotide sequence, or amino acid sequence) that is not present in the naturally occurring nucleic acid, nucleotide, protein, polypeptide, peptide, or amino acid. The terms, when referring to a composition or system described herein, refer to a composition or system having at least one component that is not naturally associated with the other components of the composition or system. By way of a non-limiting example, a composition may include an effector protein and a guide nucleic acid that do not naturally occur together. Conversely, and as a non-limiting further clarifying example, an effector protein or guide nucleic acid that is “natural,” “naturally-occurring,” or “found in nature” includes an effector protein and a guide nucleic acid from a cell or organism that have not been genetically modified by the hand of man.

The term “nucleic acid expression vector,” as used herein, refers to a plasmid that can be used to express a nucleic acid of interest.

The term “nuclear localization signal,” as used herein, refers to an entity (e.g., peptide) that facilitates localization of a nucleic acid, protein, or small molecule to the nucleus, when present in a cell that contains a nuclear compartment.

The term “nuclease activity.” as used herein, refers to the enzymatic activity of an enzyme which allows the enzyme to cleave the phosphodiester bonds between the nucleotide subunits of nucleic acids; the term “endonuclease activity” refers to the enzymatic activity of an enzyme which allows the enzyme to cleave the phosphodiester bond within a polynucleotide chain. An enzyme with nuclease activity may be referred to as a “nuclease.”

The term “pharmaceutically acceptable excipient, carrier or diluent.” as used herein, refers to any substance formulated alongside the active ingredient of a pharmaceutical composition that allows the active ingredient to retain biological activity and is non-reactive with the subject's immune system. Such a substance can be included for the purpose of long-term stabilization, bulking up solid formulations that contain potent active ingredients in small amounts, or to confer a therapeutic enhancement on the active ingredient in the final dosage form, such as facilitating absorption, reducing viscosity, or enhancing solubility. The selection of appropriate substance can depend upon the route of administration and the dosage form, as well as the active ingredient and other factors. Compositions having such substances can be formulated by well-known conventional methods (see, e.g., Remington's Pharmaceutical Sciences, 18th edition, A. Gennaro, ed., Mack Publishing Co., Easton. Pa., 1990; and Remington. The Science and Practice of Pharmacy 21st Ed. Mack Publishing, 2005).

The term “protospacer adjacent motif (PAM).” as used herein, refers to a nucleotide sequence found in a target nucleic acid that directs an effector protein to modify the target nucleic acid at a specific location. A PAM sequence may be required for a complex having an effector protein and a guide nucleic acid to hybridize to and modify the target nucleic acid. However, a given effector protein may not require a PAM sequence being present in a target nucleic acid for the effector protein to modify the target nucleic acid.

The term “recombinant.” as used herein, as applied to proteins, polypeptides, peptides and nucleic acids, refers to proteins, polypeptides, peptides and nucleic acids that are products of various combinations of cloning, restriction, and/or ligation steps resulting in a construct having a structural coding or non-coding sequence distinguishable from endogenous nucleic acids found in natural systems. Generally. DNA sequences encoding the structural coding sequence can be assembled from cDNA fragments and short oligonucleotide linkers, or from a series of synthetic oligonucleotides, to provide a synthetic nucleic acid which is capable of being expressed from a recombinant transcriptional unit contained in a cell or in a cell-free transcription and translation system. Such sequences can be provided in the form of an open reading frame uninterrupted by internal non translated sequences, or introns, which are typically present in eukaryotic genes. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions and may act to modulate production of a desired product by various mechanisms. Thus, for example, the term “recombinant polynucleotide” or “recombinant nucleic acid” refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a redundant codon encoding the same or a conservative amino acid, while typically introducing or removing a sequence recognition site. Alternatively, it is performed to join together nucleic acid segments of desired functions to generate a desired combination of functions. Similarly, the term “recombinant polypeptide” or “recombinant protein” refers to one which is not naturally occurring, e.g., is made by the artificial combination of two otherwise separated segments of amino sequences through human intervention. Thus, for example, a polypeptide that includes a heterologous amino acid sequence is a recombinant polypeptide.

In some embodiments, the term “region” as used herein may be used to describe a portion of or all of a corresponding sequence, for example, a spacer region is understood to comprise a portion of or all of a spacer sequence.

The terms “reporter” and “reporter nucleic acid” are used interchangeably herein to refer to a non-target nucleic acid molecule that can provide a detectable signal upon cleavage by an effector protein. Examples of detectable signals and detectable moieties that generate detectable signals are provided herein.

The term “sample.” as used herein, generally refers to something comprising a target nucleic acid. In some instances, the sample is a biological sample, such as a biological fluid or tissue sample. In some instances, the sample is an environmental sample. The sample may be a biological sample or environmental sample that is modified or manipulated. By way of non-limiting example, samples may be modified or manipulated with purification techniques, heat, nucleic acid amplification, salts and buffers.

The term “subject.” as used herein, refers to a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some instances, the subject is not necessarily diagnosed or suspected of being at high risk for the disease.

A “syndrome”, as used herein, refers to a group of symptoms which, taken together, characterize a condition.

The term “target nucleic acid,” as used herein, refers to a nucleic acid that is selected as the nucleic acid for modification, binding, hybridization or any other activity of or interaction with a nucleic acid, protein, polypeptide, or peptide described herein. A target nucleic acid may comprise RNA, DNA, or a combination thereof. A target nucleic acid may be single-stranded (e.g., single-stranded RNA or single-stranded DNA) or double-stranded (e.g., double-stranded DNA).

The term “target sequence,” as used herein, when used in reference to a target nucleic acid, refers to a sequence of nucleotides found within a target nucleic acid. Such a sequence of nucleotides can, for example, hybridize to an equal length portion of a guide nucleic acid. Hybridization of the guide nucleic acid to the target sequence may bring an effector protein into contact with the target nucleic acid.

The term “target nucleic acid sequence” in some contexts refers to a “target sequence” and/or a “target nucleic acid.”

The term “trans cleavage,” is used herein, in reference to cleavage (hydrolysis of a phosphodiester bond) of one or more nucleic acids by an effector protein that is complexed with a guide nucleic acid and a target nucleic acid. The one or more nucleic acids may include the target nucleic acid as well as non-target nucleic acids.

The term “trans-activating RNA (tracrRNA),” as used herein, refers to a nucleic acid that comprises a first sequence that is capable of being non-covalently bound by an effector protein. TracrRNAs may comprise a second sequence that hybridizes to a portion of a crRNA, which may be referred to as a repeat hybridization sequence. In some embodiments, tracrRNA sequences are covalently linked to a crRNA.

The term “transcriptional activator,” as used herein, refers to a polypeptide or a fragment thereof that can activate or increase transcription of a target nucleic acid molecule.

The term “transcriptional repressor,” as used herein, refers to a polypeptide or a fragment thereof that is capable of arresting, preventing, or reducing transcription of a target nucleic acid.

The terms “treatment” or “treating,” as used herein, are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying, or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The term “viral vector,” as used herein, refers to a nucleic acid to be delivered into a host cell via a recombinantly produced virus or viral particle. The nucleic acid may be single-stranded or double stranded, linear or circular, segmented or non-segmented. The nucleic acid may comprise DNA, RNA, or a combination thereof. Non-limiting examples of viruses or viral particles that can deliver a viral vector include retroviruses (e.g., lentiviruses and γ-retroviruses), adenoviruses, arenaviruses, alphaviruses, adeno-associated viruses (AAVs), baculoviruses, vaccinia viruses, herpes simplex viruses and poxviruses. A viral vector delivered by such viruses or viral particles may be referred to by the type of virus to deliver the viral vector (e.g., an AAV viral vector is a viral vector that is to be delivered by an adeno-associated virus). A viral vector referred to by the type of virus to be delivered by the viral vector can contain viral elements (e.g., nucleotide sequences) necessary for packaging of the viral vector into the virus or viral particle, replicating the virus, or other desired viral activities. A virus containing a viral vector may be replication competent, replication deficient or replication defective.

### INTRODUCTION

Disclosed herein are compositions, systems and methods comprising at least one of:
a) a polypeptide or a nucleic acid encoding the polypeptide; and
b) a guide nucleic acid or a nucleic acid encoding the guide nucleic acid.

Polypeptides described herein may bind and, optionally, cleave nucleic acids in a sequence-specific manner. Polypeptides described herein may also cleave the target nucleic acid within a target sequence or at a position adjacent to the target sequence. In some embodiments, a polypeptide is activated when it binds a certain sequence of a nucleic acid described herein, allowing the polypeptide to cleave a region of a target nucleic acid that is near, but not adjacent to the target sequence. A polypeptide may be an effector protein, such as a CRISPR-associated (Cas) protein, which may bind a guide nucleic acid that imparts activity or sequence selectivity to the polypeptide. An effector protein may also be referred to as a programmable nuclease because the nuclease activity of the protein may be directed to different target nucleic acids by way of revising the guide nucleic acid that the protein binds.

Programmable nucleases are proteins that bind and cleave nucleic acids in a sequence-specific manner. A programmable nuclease may bind a target region of a nucleic acid and cleave the nucleic acid within the target region or at a position adjacent to the target region. In some instances, a programmable nuclease is activated when it binds a target region of a nucleic acid to cleave regions of the nucleic acid that are near, but not adjacent to the target region. A programmable nuclease, such as a CRISPR-associated (Cas) protein, may be coupled to a guide nucleic acid that imparts activity or sequence selectivity to the programmable nuclease.

In some embodiments, compositions, systems, and methods comprising guide nucleic acids comprise a first sequence, at least a portion of which interacts with a polypeptide. In some embodiments, the first sequence comprises a sequence that is similar or identical to an intermediary nucleic acid sequence, a repeat sequence, or a combination thereof. In some embodiments, the guide nucleic acid does not comprise an intermediary nucleic acid. In some embodiments, compositions, systems, and methods guide nucleic acids comprise a second sequence that is at least partially complementary to a target nucleic acid, and which may be referred to as a spacer sequence.

In general, guide nucleic acids comprise a CRISPR RNA (crRNA) that is at least partially complementary to a target nucleic acid. In some cases, a composition, systems and methods comprising effector proteins and guide nucleic acids further comprise a trans-activating crRNA (tracrRNA), at least a portion of which interacts with the programmable nuclease. In some cases, a tracrRNA is provided separately from the guide nucleic acid. The tracrRNA may hybridize to a portion of the guide nucleic acid that does not hybridize to the target nucleic acid.

Programmable nucleases may cleave nucleic acids, including single stranded RNA (ssRNA), double stranded DNA (dsDNA), and single-stranded DNA (ssDNA). Programmable nucleases may provide cis cleavage activity, trans cleavage activity, nickase activity, or a combination thereof. Cis cleavage activity is cleavage of a target nucleic acid that is hybridized to a guide RNA (crRNA or sgRNA), wherein cleavage occurs within or directly adjacent to the region of the target nucleic acid that is hybridized to guide RNA. Trans cleavage activity (also referred to as transcollateral cleavage) is cleavage of ssDNA or ssRNA that is near, but not hybridized to the guide RNA. Trans cleavage activity is triggered by the hybridization of guide RNA to the target nucleic acid. Nickase activity is the selective cleavage of one strand of a dsDNA molecule. Programmable CRISPR-associated (Cas) nucleases, through their ability to cleave DNA at a precise target location in the genome of a wide variety of cells and organisms, allow for precise and efficient editing of DNA sequences of interest. SSBs and DSBs are an effective way to disrupt a gene of interest, generate DNA or RNA modifications, and to treat genetic disease through gene correction.

Disclosed herein are non-naturally occurring compositions, methods and systems comprising at least one of an engineered effector protein and an engineered guide nucleic acid (which may simply be referred to herein as an effector protein and a guide nucleic acid, respectively), or a use thereof. In general, an effector protein and a guide nucleic acid refer to an effector protein and a guide nucleic acid, respectively, that are not found in nature. In some embodiments, compositions, systems and methods comprise an engineered protein or a use thereof. In some embodiments, compositions, systems and methods comprise an isolated polypeptide or a use thereof. In some embodiments, systems, methods and compositions herein comprise at least one non-naturally occurring component. For example, compositions, methods and systems may comprise a guide nucleic acid, wherein the sequence of the guide nucleic acid is different or modified from that of a naturally-occurring guide nucleic acid.

In some embodiments, compositions, methods and systems comprise at least two components that do not naturally occur together. For example, compositions, methods and systems may comprise a guide nucleic acid comprising a repeat region and a spacer region which do not naturally occur together. Similarly, disclosed compositions, systems and methods may comprise a guide nucleic acid comprising a second region, at least a portion of which, interacts with a polypeptide, and a first region that is at least partially complementary to a target nucleic acid, wherein the first region and second region do not naturally occur together. Also, by way of example, compositions, methods and systems may comprise a guide nucleic acid and an effector protein that do not naturally occur together. Likewise, by way of non-limiting example, disclosed compositions, systems, and methods may comprise a ribonucleotide-protein (RNP) complex comprising an effector protein and a guide nucleic acid that do not occur together in nature. Conversely, and for clarity, an effector protein or guide nucleic acid that is “natural.” “naturally-occurring.” or “found in nature” includes effector proteins and guide nucleic acids from cells or organisms that have not been genetically modified by a human or machine.

In some embodiments, the guide nucleic acid comprises a non-natural nucleobase sequence. In some embodiments, the non-natural sequence is a nucleobase sequence that is not found in nature. The non-natural sequence may comprise a portion of a naturally-occurring sequence, wherein the portion of the naturally-occurring sequence is not present in nature absent the remainder of the naturally-occurring sequence. In some embodiments, the guide nucleic acid comprises two naturally-occurring sequences arranged in an order or proximity that is not observed in nature. In some embodiments, compositions, methods and systems comprise a ribonucleotide complex comprising an effector protein and a guide nucleic acid that do not occur together in nature. In some embodiments, when describing a ribonucleotide complex (RNP) reference is made to a complex of one or more nucleic acids and one or more polypeptides described herein. While the term utilizes “ribonucleotides” it is understood that the one or more nucleic acid may comprise deoxyribonucleotides (DNA), ribonucleotides (RNA), a combination thereof (e.g., RNA with a thymine base), biochemically or chemically modified nucleobases (e.g., one or more engineered modifications described herein), or combinations thereof.

Engineered guide nucleic acids may comprise a first sequence and a second sequence that do not occur naturally together. For example, a guide nucleic acid may comprise a sequence of a naturally-occurring repeat region and a spacer region that is complementary to a naturally-occurring eukaryotic sequence. The guide nucleic acid may comprise a sequence of a repeat region that occurs naturally in an organism and a spacer region that does not occur naturally in that organism. A guide nucleic acid may comprise a first sequence that occurs in a first organism and a second sequence that occurs in a second organism, wherein the first organism and the second organism are different. The guide nucleic acid may comprise a third sequence disposed at a 3′ or 5′ end of the guide nucleic acid, or between the first and second sequences of the guide nucleic acid. For example, a guide nucleic acid may comprise a crRNA and tracrRNA sequence coupled by a linker sequence. In some embodiments, the guide nucleic acid comprises two heterologous sequences arranged in an order or proximity that is not observed in nature. Therefore, compositions, methods and systems described herein are not naturally occurring.

In some embodiments, compositions, methods and systems described herein comprise an effector protein that is similar to a naturally occurring effector protein. The effector protein may lack a portion of the naturally occurring effector protein. The effector protein may comprise a mutation relative to the naturally-occurring effector protein, wherein the mutation is not found in nature. The effector protein may also comprise at least one additional amino acid relative to the naturally-occurring effector protein. In some embodiments, the effector protein may comprise a heterologous polypeptide. For example, the effector protein may comprise an addition of a nuclear localization signal relative to the natural occurring effector protein. In some embodiments compositions, methods and systems described herein may comprise one or more nuclear localization signals (NLS). In some embodiments, compositions, methods and systems described herein may comprise a NLS sequence that is adjacent to the N terminal of the effector protein sequence or that is adjacent to the C terminal of the effector protein sequence, or both. In certain embodiments, the nucleotide sequence encoding the effector protein is codon optimized (e.g., for expression in a eukaryotic cell) relative to the naturally occurring sequence.

### I. Polypeptide Systems

### Effector Proteins

Provided herein, in certain embodiments, are compositions, methods and systems that comprise one or more effector proteins or a use thereof.

An effector protein provided herein interacts with a guide nucleic acid to form a complex. In some embodiments, the complex interacts with a target nucleic acid. An effector protein may be brought into proximity of a target nucleic acid in the presence of a guide nucleic acid when the guide nucleic acid includes a nucleotide sequence that is complementary with a target sequence in the target nucleic acid. In some embodiments, an interaction between the complex and a target nucleic acid comprises one or more of: recognition of a protospacer adjacent motif (PAM) sequence within the target nucleic acid by the effector protein, hybridization of the guide nucleic acid to the target nucleic acid, modification of the target nucleic acid by the effector protein, or combinations thereof. The ability of an effector protein to modify a target nucleic acid may be dependent upon the effector protein being bound to a guide nucleic acid and the guide nucleic acid being hybridized to a target nucleic acid. An effector protein may also recognize a protospacer adjacent motif (PAM) sequence present in the target nucleic acid. In some embodiments, recognition of a PAM sequence within a target nucleic acid may direct the modification activity of an effector protein. In some embodiments, recognition of a PAM sequence adjacent to a target sequence in a target nucleic acid may direct the modification activity of an effector protein.

Modification activity of an effector protein or an engineered protein described herein may be cleavage activity, binding activity, insertion activity, substitution activity, and the like. Modification activity of an effector protein may result in: cleavage of at least one strand of a target nucleic acid, deletion of one or more nucleotides of a target nucleic acid, insertion of one or more nucleotides into a target nucleic acid, substitution of one or more nucleotides of a target nucleic acid with an alternative nucleotide, more than one of the foregoing, or any combination thereof. In some embodiments, an ability of an effector protein to edit a target nucleic acid may depend upon the effector protein being complexed with a guide nucleic acid, the guide nucleic acid being hybridized to a target sequence of the target nucleic acid, the distance between the target sequence and a PAM sequence, or combinations thereof. A target nucleic acid comprises a target strand and a non-target strand. Accordingly, in some embodiments, the effector protein may edit a target strand and/or a non-target strand of a target nucleic acid.

The modification of the target nucleic acid generated by an effector protein may, as a non-limiting example, result in modulation of the expression of the nucleic acid (e.g., increasing or decreasing expression of the nucleic acid) or modulation of the activity of a translation product of the target nucleic acid (e.g., inactivation of a protein binding to an RNA molecule or hybridization). In some embodiments, provided herein are methods of editing a target nucleic acid using an effector protein of the present disclosure, or compositions or systems thereof. Also provided herein are methods of modulating expression of a target nucleic acid using an effector protein of the present disclosure, or compositions or systems thereof. Further provided herein are methods of modulating the activity of a translation product of a target nucleic acid using an effector protein of the present disclosure, or compositions or systems thereof.

An effector protein may modify a nucleic acid by cis cleavage or trans cleavage. In some embodiments, effector proteins disclosed herein may provide cleavage activity, such as cis cleavage activity, trans cleavage activity, nickase activity, nuclease activity, or a combination thereof. In general, effector proteins described herein edit a target nucleic acid by cis cleavage activity on the target nucleic acid. Effector proteins disclosed herein may cleave nucleic acids, including single stranded RNA (ssRNA), double stranded DNA (dsDNA), and single-stranded DNA (ssDNA).

An effector protein may be a CRISPR-associated (“Cas”) protein. An effector protein may function as a single protein, including a single protein that is capable of binding to a guide nucleic acid and modifying a target nucleic acid. Alternatively, an effector protein may function as part of a multiprotein complex, including, for example, a complex having two or more effector proteins, including two or more of the same effector proteins (e.g., dimer or multimer). An effector protein, when functioning in a multiprotein complex, may have only one functional activity (e.g., binding to a guide nucleic acid), while other effector proteins present in the multiprotein complex are capable of the other functional activity (e.g., modifying a target nucleic acid). In some embodiments, an effector protein, when functioning in a multiprotein complex, may have differing and/or complementary functional activity to other effector proteins in the multiprotein complex. Multimeric complexes, and functions thereof, are described in further detail below.

In some instances, the effector proteins function as an endonuclease that catalyzes cleavage within a target nucleic acid. In some instances, the effector proteins are capable of catalyzing non-sequence-specific cleavage of a single stranded nucleic acid. In some instances, the effector proteins (e.g., the effector proteins having the sequence of TABLE 1) are activated to perform trans cleavage activity after binding of a guide nucleic acid with a target nucleic acid. This trans cleavage activity may also be referred to as “collateral” or “transcollateral” cleavage. Trans cleavage activity may be non-specific cleavage of nearby single-stranded nucleic acid by the activated effector protein, such as trans cleavage of detector nucleic acids with a detection moiety.

Effector proteins disclosed herein may function as an endonuclease that catalyzes cleavage at a specific position (e.g., at a specific nucleotide within a nucleic acid sequence) in a target nucleic acid. The target nucleic acid may be single stranded RNA (ssRNA), double stranded DNA (dsDNA) or single-stranded DNA (ssDNA). In some instances, the target nucleic acid is single-stranded DNA. In some instances, the target nucleic acid is single-stranded RNA. The effector proteins may provide cis cleavage activity, trans cleavage activity, nickase activity, or a combination thereof, cis cleavage activity is cleavage of a target nucleic acid that is hybridized to a guide RNA (e.g., a dual guide nucleic acid system or a sgRNA), wherein cleavage occurs within or directly adjacent to the region of the target nucleic acid that is hybridized to guide RNA. Trans cleavage activity (also referred to as transcollateral cleavage) is cleavage of ssDNA or ssRNA that is near, but not hybridized to the guide RNA, trans cleavage may occur near, but not within or directly adjacent to, the region of the target nucleic acid that is hybridized to the guide nucleic acid. Trans cleavage activity may be triggered by the hybridization of the guide nucleic acid to the target nucleic acid. Nickase activity is a selective cleavage of one strand of a dsDNA.

An effector protein may be a modified effector protein having increased modification activity and/or increased substrate binding activity (e.g., substrate selectivity, specificity, and/or affinity). An effector protein may be a modified effector protein having reduced modification activity (e.g., a catalytically defective effector protein) or no modification activity (e.g., a catalytically inactive effector protein). Accordingly, an effector protein as used herein encompasses a modified or programmable nuclease that does not have nuclease activity.

TABLE 1 provides an illustrative amino acid sequence of effector proteins that are useful in the compositions, systems and methods described herein.

In some instances, an effector protein provided herein is at least 65%, at least 70%, at least 75%, at least 80%, at least 85%, at least 90%, at least 95%, at least 97%, or at least 98%, at least 99%, or 100% identical to any one of the sequences set forth in TABLE 1. In some embodiments, an effector protein provided herein is at least 65% identical to any one of the sequences set forth in TABLE 1. In some embodiments, an effector protein provided herein is at least 70% identical to any one of the sequences set forth in TABLE 1. In some embodiments, an effector protein provided herein is at least 75% identical to any one of the sequences set forth in TABLE 1. In some embodiments, an effector protein provided herein is at least 80% identical to any one of the sequences set forth in TABLE 1. In some embodiments, an effector protein provided herein is at least 85% identical to any one of the sequences set forth in TABLE 1. In some embodiments, an effector protein provided herein is at least 90% identical to any one of the sequences set forth in TABLE 1. In some embodiments, an effector protein provided herein is at least 95% identical to any one of the sequences set forth in TABLE 1. In some embodiments, an effector protein provided herein is at least 97% identical to any one of the sequences set forth in TABLE 1. In some embodiments, an effector protein provided herein is at least 98% identical to any one of the sequences set forth in TABLE 1. In some embodiments, an effector protein provided herein is at least 99% identical to any one of the sequences set forth in TABLE 1. In some embodiments, an effector protein provided herein is 100% identical to any one of the sequences set forth in TABLE 1.

In certain instances, compositions, systems and methods described herein comprise an effector protein, or a nucleic acid encoding an effector protein, wherein the effector protein comprises an amino acid sequence that is at least 65%, at least 70%, at least 75%, at least 80%, at least 85%, at least 90%, at least 95%, at least 98%, at least 99%, or 100% identical to any one of the sequences set forth in TABLE 1. In some embodiments, an effector protein provided herein comprises an amino acid sequence that is at least 65% identical to any one of the sequences set forth in TABLE 1. In some embodiments, an effector protein provided herein comprises an amino acid sequence that is at least 70% identical to any one of the sequences set forth in TABLE 1. In some embodiments, an effector protein provided herein comprises an amino acid sequence that is at least 75% identical to any one of the sequences set forth in TABLE 1. In some embodiments, an effector protein provided herein comprises an amino acid sequence that is at least 80% identical to any one of the sequences set forth in TABLE 1. In some embodiments, an effector protein provided herein comprises an amino acid sequence that is at least 85% identical to any one of the sequences set forth in TABLE 1. In some embodiments, an effector protein provided herein comprises an amino acid sequence that is at least 90% identical to any one of the sequences set forth in TABLE 1. In some embodiments, an effector protein provided herein comprises an amino acid sequence that is at least 95% identical to any one of the sequences set forth in TABLE 1. In some embodiments, an effector protein provided herein comprises an amino acid sequence that is at least 97% identical to any one of the sequences set forth in TABLE 1. In some embodiments, an effector protein provided herein comprises an amino acid sequence that is at least 98% identical to any one of the sequences set forth in TABLE 1. In some embodiments, an effector protein provided herein comprises an amino acid sequence that is at least 99% identical to any one of the sequences set forth in TABLE 1. In some embodiments, an effector protein provided herein comprises an amino acid sequence that is 100% identical to any one of the sequences set forth in TABLE 1.

In some instances, the amino acid sequence of the effector protein is at least 65%, at least 70%, at least 75%, at least 80%, at least 85%, at least 90%, at least 95%, at least 97%, or at least 98%, at least 99%, or 100% identical to any one of the sequences set forth in TABLE 1. In some embodiments, the amino acid sequence of an effector protein provided herein is at least 65% identical to any one of the sequences set forth in TABLE 1. In some embodiments, the amino acid sequence of an effector protein provided herein is at least 70% identical to any one of the sequences set forth in TABLE 1. In some embodiments, the amino acid sequence of an effector protein provided herein is at least 75% identical to any one of the sequences set forth in TABLE 1. In some embodiments, the amino acid sequence of an effector protein provided herein is at least 80% identical to any one of the sequences set forth in TABLE 1. In some embodiments, the amino acid sequence of an effector protein provided herein is at least 85% identical to any one of the sequences set forth in TABLE 1. In some embodiments, the amino acid sequence of an effector protein provided herein is at least 90% identical to any one of the sequences set forth in TABLE 1. In some embodiments, the amino acid sequence of an effector protein provided herein is at least 95% identical to any one of the sequences set forth in TABLE 1. In some embodiments, the amino acid sequence of an effector protein provided herein is at least 97% identical to any one of the sequences set forth in TABLE 1. In some embodiments, the amino acid sequence of an effector protein provided herein is at least 98% identical to any one of the sequences set forth in TABLE 1. In some embodiments, the amino acid sequence of an effector protein provided herein is at least 99% identical to any one of the sequences set forth in TABLE 1. In some embodiments, the amino acid sequence of an effector protein provided herein is 100% identical to any one of the sequences set forth in TABLE 1.

In some embodiments, compositions, systems, and methods described herein comprise an effector protein, or a nucleic acid encoding the effector protein, wherein the effector protein comprises an amino acid sequence that is at least 80%, at least 85%, at least 90%, at least 95%, at least 97%, at least 98%, at least 99%, or 100% similar to any one of the sequences as set forth in TABLE 1. In some embodiments, an effector protein provided herein comprises an amino acid sequence that is at least 80% similar to any one of the sequences as set forth in TABLE 1. In some embodiments, an effector protein provided herein comprises an amino acid sequence that is at least 85% similar to any one of the sequences as set forth in TABLE 1. In some embodiments, an effector protein provided herein comprises an amino acid sequence that is at least 90% identical to any one of the sequences as set forth in TABLE 1. In some embodiments, an effector protein provided herein comprises an amino acid sequence that is at least 95% similar to any one of the sequences as set forth in TABLE 1. In some embodiments, an effector protein provided herein comprises an amino acid sequence that is at least 97% similar to any one of the sequences as set forth in TABLE 1. In some embodiments, an effector protein provided herein comprises an amino acid sequence that is at least 98% identical to any one of the sequences as set forth in TABLE 1. In some embodiments, an effector protein provided herein comprises an amino acid sequence that is at least 99% similar to any one of the sequences as set forth in TABLE 1. In some embodiments, an effector protein provided herein comprises an amino acid sequence that is 100% similar to any one of the sequences as set forth in TABLE 1.

In some embodiments, when describing percent similarity, in the context of an amino acid sequence, reference is made to a value that is calculated by dividing a similarity score by the length of the alignment. The similarity of two amino acid sequences can be calculated by using a BLOSUM62 similarity matrix (Henikoff and Henikoff. Proc. Natl. Acad. Sci. USA., 89:10915-10919 (1992)) that is transformed so that any value ≥1 is replaced with +1 and any value ≤0 is replaced with 0. For example, an Ile (I) to Leu (L) substitution is scored at +2.0 by the BLOSUM62 similarity matrix, which in the transformed matrix is scored at +1. This transformation allows the calculation of percent similarity, rather than a similarity score. Alternately, when comparing two full protein sequences, the proteins can be aligned using pairwise MUSCLE alignment. Then, the % similarity can be scored at each residue and divided by the length of the alignment. For determining % similarity over a protein domain or motif, a multilevel consensus sequence (or PROSITE motif sequence) can be used to identify how strongly each domain or motif is conserved. In calculating the similarity of a domain or motif, the second and third levels of the multilevel sequence are treated as equivalent to the top level. Additionally, if a substitution could be treated as conservative with any of the amino acids in that position of the multilevel consensus sequence, +1 point is assigned. For example, given the multilevel consensus sequence: RLG and YCK, the test sequence QIQ would receive three points. This is because in the transformed BLOSUM62 matrix, each combination is scored as: Q-R: +1; Q-Y: +0; I-L: +1; I-C: +0; Q-G: +0; Q-K: +1 For each position, the highest score is used when calculating similarity. The % similarity can also be calculated using commercially available programs, such as the Gencious Prime software given the parameters matrix=BLOSUM62 and threshold ≥1.

In some embodiments, compositions, systems and methods described herein comprise an effector protein, or a nucleic acid encoding the effector protein, wherein the amino acid sequence of the effector protein comprises at least about 200 contiguous amino acids or more of any one of the sequences recited in TABLE 1. In some embodiments, the amino acid sequence of an effector protein provided herein comprises at least about 200, at least about 220, at least about 240, at least about 260, at least about 280, at least about 300, at least about 320, at least about 340, at least about 360, at least about 380, at least about 400 contiguous amino acids, at least about 420 contiguous amino acids, at least about 440 contiguous amino acids, at least about 460 contiguous amino acids, at least about 480 contiguous amino acids, at least about 500 contiguous amino acids, at least about 520 contiguous amino acids, at least about 540 contiguous amino acids, at least about 560 contiguous amino acids, at least about 580 contiguous amino acids, at least about 600 contiguous amino acids, at least about 620 contiguous amino acids, at least about 640 contiguous amino acids, at least about 660 contiguous amino acids, at least about 680 contiguous amino acids, at least about 700 contiguous amino acids, or more of any one of the sequences of TABLE 1.

In certain instances, compositions, systems and methods described herein comprise an effector protein and an engineered guide nucleic acid, wherein the amino acid sequence of the effector protein comprises at least about 200, at least about 220, at least about 240, at least about 260, at least about 280, at least about 300, at least about 320, at least about 340, at least about 360, at least about 380, or at least about 400 contiguous amino acids or more of any one of the sequences as set forth in TABLE 1. In certain instances, compositions, systems and methods described herein comprise an effector protein and an engineered guide nucleic acid, wherein the amino acid sequence of the effector protein comprises at least about 200 contiguous amino acids or more of any one of the sequences as set forth in TABLE 1. In certain instances, compositions, systems and methods described herein comprise an effector protein and an engineered guide nucleic acid, wherein the amino acid sequence of the effector protein comprises at least about 300 contiguous amino acids or more of any one of the sequences as set forth in TABLE 1. In certain instances, compositions, systems and methods described herein comprise an effector protein and an engineered guide nucleic acid, wherein the amino acid sequence of the effector protein comprises at least about 400 contiguous amino acids or more of any one of the sequences as set forth in TABLE 1.

In some embodiments, compositions, systems and methods described herein comprise an effector protein or a nucleic acid encoding the effector protein, wherein the effector protein comprises a portion of any one of the sequences recited in TABLE 1. In some embodiments, the effector protein comprises a portion of any one of the sequences recited in TABLE 1, wherein the portion does not comprise at least the first 10 amino acids, at least the first 20 amino acids, at least the first 40 amino acids, at least the first 60 amino acids, at least the first 80 amino acids, at least the first 100 amino acids, at least the first 120 amino acids, at least the first 140 amino acids, at least the first 160 amino acids, at least the first 180 amino acids, or at least the first 200 amino acids of any one of the sequences recited in TABLE 1. In some embodiments, the effector protein comprises a portion of any one of the sequences recited in TABLE 1, wherein the portion does not comprise the last 10 amino acids, the last 20 amino acids, the last 40 amino acids, the last 60 amino acids, the last 80 amino acids, the last 100 amino acids, the last 120 amino acids, the last 140 amino acids, the last 160 amino acids, the last 180 amino acids, or the last 200 amino acids of any one of the sequences recited in TABLE 1.

In some cases, the effector proteins comprise a RuvC domain. In some instances, the RuvC domain may be defined by a single, contiguous sequence, or a set of RuvC subdomains that are not contiguous with respect to the primary amino acid sequence of the protein. An effector protein of the present disclosure may include multiple RuvC subdomains, which may combine to generate a RuvC domain with substrate binding or catalytic activity. For example, an effector protein may include three RuvC subdomains (RuvC-I. RuvC-II, and RuvC-III) that are not contiguous with respect to the primary amino acid sequence of the effector protein, but form a RuvC domain once the protein is produced and folds. In many cases, effector proteins comprise a recognition domain with a binding affinity for a guide nucleic acid or for a guide nucleic acid-target nucleic acid heteroduplex. An effector protein may comprise a zinc finger domain.

An effector protein may be small, which may be beneficial for nucleic acid detection or editing (for example, the effector protein may be less likely to adsorb to a surface or another biological species due to its small size). The smaller nature of these effector proteins may allow for them to be more easily packaged and delivered with higher efficiency in the context of genome editing and more readily incorporated as a reagent in an assay. In some instances, the length of the effector protein is at least 400 linked amino acid residues. In some instances, the length of the effector protein is less than 500 linked amino acid residues. In some instances, the length of the effector protein is about 400 to about 500 linked amino acid residues. In some instances, the length of the effector protein is about 450 to about 550, about 400 to about 420, about 420 to about 440, about 440 to about 460, about 460 to about 480, about 480 to about 500, about 500 to about 520, about 520 to about 540, about 540 to about 560, about 560 to about 580, about 580 to about 600, about 600 to about 620, about 620 to about 640, about 640 to about 660, about 660 to about 680, about 680 to about 700 linked amino acids.

In some embodiments, compositions, systems, and methods described herein comprise an effector protein, or a nucleic acid encoding the effector protein, wherein the effector protein comprises one or more amino acid alterations relative to any one of the sequences recited in TABLE 1. In some embodiments, the effector protein comprising one or more amino acid alterations is a variant of an effector protein described herein. It is understood that any reference to an effector protein herein also refers to an effector protein variant as described herein. In some embodiments, the one or more amino acid alterations comprises conservative substitutions, non-conservative substitutions, conservative deletions, non-conservative deletions, or combinations thereof. In some embodiments, an effector protein or a nucleic acid encoding the effector protein comprises 1 amino acid alteration, 2 amino acid alterations, 3 amino acid alterations, 4 amino acid alterations, 5 amino acid alterations, 6 amino acid alterations, 7 amino acid alterations, 8 amino acid alterations, 9 amino acid alterations, 10 amino acid alterations or more relative to any one of the sequences recited in TABLE 1.

In some embodiments, when describing a conservative substitution reference is made to the replacement of one amino acid for another such that the replacement takes place within a family of amino acids that are related in their side chains. Conversely, the term “non-conservative substitution” as used herein refers to the replacement of one amino acid residue for another that does not have a related side chain. Genetically encoded amino acids can be divided into four families having related side chains: (1) acidic (negatively charged): Asp (D). Glu (E); (2) basic (positively charged): Lys (K). Arg (R), His (H); (3) non-polar (hydrophobic): Cys (C), Ala (A), Val (V), Leu (L), Ile (I), Pro (P), Phc (F), Mct (M), Trp (W), Gly (G), Tyr (Y), with non-polar also being subdivided into: (i) strongly hydrophobic: Ala (A), Val (V), Leu (L), Ile (I), Met (M), Phe (F); and (ii) moderately hydrophobic: Gly (G), Pro (P), Cys (C), Tyr (Y), Trp (W); and (4) uncharged polar: Asn (N), Gln (Q), Ser(S), Thr (T), Amino acids may be related by aliphatic side chains: Gly (G), Ala (A), Val (V), Leu (L), Ile (I), Ser(S), Thr (T), with Ser(S) and Thr (T) optionally being grouped separately as aliphatic-hydroxyl; Amino acids may be related by aromatic side chains: Phe (F). Tyr (Y). Trp (W). Amino acids may be related by amide side chains: Asn (N), Gln (Q), Amino acids may be related by sulfur-containing side chains: Cys (C) and Met (M).

In some instances, other than one or more alterations set forth in TABLE 1.1, an effector protein provided herein is at least 65%, at least 70%, at least 75%, at least 80%, at least 85%, at least 90%, at least 95%, at least 97%, or at least 98%, at least 99%, or 100% identical to any one of the sequences set forth in TABLE 1. In some embodiments, other than one or more alterations set forth in TABLE 1.1, an effector protein provided herein is at least 65% identical to any one of the sequences set forth in TABLE 1. In some embodiments, other than one or more alterations set forth in TABLE 1.1, an effector protein provided herein is at least 70% identical to any one of the sequences set forth in TABLE 1. In some embodiments, other than one or more alterations set forth in TABLE 1.1, an effector protein provided herein is at least 75% identical to any one of the sequences set forth in TABLE 1. In some embodiments, other than one or more alterations set forth in TABLE 1.1, an effector protein provided herein is at least 80% identical to any one of the sequences set forth in TABLE 1. In some embodiments, other than one or more alterations set forth in TABLE 1.1, an effector protein provided herein is at least 85% identical to any one of the sequences set forth in TABLE 1. In some embodiments, other than one or more alterations set forth in TABLE 1.1, an effector protein provided herein is at least 90% identical to any one of the sequences set forth in TABLE 1. In some embodiments, other than one or more alterations set forth in TABLE 1.1, an effector protein provided herein is at least 95% identical to any one of the sequences set forth in TABLE 1. In some embodiments, other than one or more alterations set forth in TABLE 1.1, an effector protein provided herein is at least 97% identical to any one of the sequences set forth in TABLE 1. In some embodiments, other than one or more alterations set forth in TABLE 1.1, an effector protein provided herein is at least 98% identical to any one of the sequences set forth in TABLE 1. In some embodiments, other than one or more alterations set forth in TABLE 1.1, an effector protein provided herein is at least 99% identical to any one of the sequences set forth in TABLE 1. In some embodiments, other than one or more alterations set forth in TABLE 1.1, an effector protein provided herein is 100% identical to any one of the sequences set forth in TABLE 1.

In certain instances, compositions, systems and methods described herein comprise an effector protein, or a nucleic acid encoding an effector protein, wherein the effector protein comprises an amino acid sequence that is at least 65%, at least 70%, at least 75%, at least 80%, at least 85%, at least 90%, at least 95%, at least 98%, at least 99%, identical to any one of the sequences set forth in TABLE 1 and comprises one or more alterations set forth in TABLE 1.1. In some embodiments, an effector protein provided herein comprises an amino acid sequence that is at least 65% identical to any one of the sequences set forth in TABLE 1, and comprises one or more alterations set forth in TABLE 1.1. In some embodiments, an effector protein provided herein comprises an amino acid sequence that is at least 70% identical to any one of the sequences set forth in TABLE 1, and comprises one or more alterations set forth in TABLE 1.1. In some embodiments, an effector protein provided herein comprises an amino acid sequence that is at least 75% identical to any one of the sequences set forth in TABLE 1, and comprises one or more alterations set forth in TABLE 1.1. In some embodiments, an effector protein provided herein comprises an amino acid sequence that is at least 80% identical to any one of the sequences set forth in TABLE 1, and comprises one or more alterations set forth in TABLE 1.1. In some embodiments, an effector protein provided herein comprises an amino acid sequence that is at least 85% identical to any one of the sequences set forth in TABLE 1, and comprises one or more alterations set forth in TABLE 1.1. In some embodiments, an effector protein provided herein comprises an amino acid sequence that is at least 90% identical to any one of the sequences set forth in TABLE 1, and comprises one or more alterations set forth in TABLE 1.1. In some embodiments, an effector protein provided herein comprises an amino acid sequence that is at least 95% identical to any one of the sequences set forth in TABLE 1, and comprises one or more alterations set forth in TABLE 1.1. In some embodiments, an effector protein provided herein comprises an amino acid sequence that is at least 97% identical to any one of the sequences set forth in TABLE 1, and comprises one or more alterations set forth in TABLE 1.1. In some embodiments, an effector protein provided herein comprises an amino acid sequence that is at least 98% identical to any one of the sequences set forth in TABLE 1, and comprises one or more alterations set forth in TABLE 1.1. In some embodiments, an effector protein provided herein comprises an amino acid sequence that is at least 99% identical to any one of the sequences set forth in TABLE 1, and comprises one or more alterations set forth in TABLE 1.1.

In some instances, other than one or more alterations set forth in TABLE 1.1, the amino acid sequence of the effector protein is at least 65%, at least 70%, at least 75%, at least 80%, at least 85%, at least 90%, at least 95%, at least 97%, or at least 98%, at least 99%, or 100% identical to any one of the sequences set forth in TABLE 1. In some embodiments, other than one or more alterations set forth in TABLE 1.1, the amino acid sequence of an effector protein provided herein is at least 65% identical to any one of the sequences set forth in TABLE 1. In some embodiments, other than one or more alterations set forth in TABLE 1.1, the amino acid sequence of an effector protein provided herein is at least 70% identical to any one of the sequences set forth in TABLE 1. In some embodiments, other than one or more alterations set forth in TABLE 1.1, the amino acid sequence of an effector protein provided herein is at least 75% identical to any one of the sequences set forth in TABLE 1. In some embodiments, other than one or more alterations set forth in TABLE 1.1, the amino acid sequence of an effector protein provided herein is at least 80% identical to any one of the sequences set forth in TABLE 1. In some embodiments, other than one or more alterations set forth in TABLE 1.1, the amino acid sequence of an effector protein provided herein is at least 85% identical to any one of the sequences set forth in TABLE 1. In some embodiments, other than one or more alterations set forth in TABLE 1.1, the amino acid sequence of an effector protein provided herein is at least 90% identical to any one of the sequences set forth in TABLE 1. In some embodiments, other than one or more alterations set forth in TABLE 1.1, the amino acid sequence of an effector protein provided herein is at least 95% identical to any one of the sequences set forth in TABLE 1. In some embodiments, other than one or more alterations set forth in TABLE 1.1, the amino acid sequence of an effector protein provided herein is at least 97% identical to any one of the sequences set forth in TABLE 1. In some embodiments, other than one or more alterations set forth in TABLE 1.1, the amino acid sequence of an effector protein provided herein is at least 98% identical to any one of the sequences set forth in TABLE 1. In some embodiments, other than one or more alterations set forth in TABLE 1.1, the amino acid sequence of an effector protein provided herein is at least 99% identical to any one of the sequences set forth in TABLE 1.

In some embodiments, the one or more amino acid alterations may result in a change in activity of the effector protein relative to a naturally-occurring counterpart. For example, and as described in further detail below, the one or more amino acid alteration increases or decreases catalytic activity of the effector protein relative to a naturally-occurring counterpart. In some embodiments, the one or more amino acid alterations results in a catalytically inactive effector protein variant.

### Engineered Proteins

In some embodiments, effector proteins described herein have been modified (also referred to as an engineered protein). In some embodiments, a modification of the effector proteins may include addition of one or more amino acids, deletion of one or more amino acids, substitution of one or more amino acids, or combinations thereof. In some instances, effector proteins disclosed herein are engineered proteins. Unless otherwise indicated, reference to effector proteins throughout the present disclosure include engineered proteins thereof. Engineered proteins are not identical to a naturally-occurring protein.

In some embodiments, effector proteins described herein can be modified with the addition of one or more heterologous peptides or heterologous polypeptides (referred to collectively herein as a heterologous polypeptide). In some embodiments, an effector protein modified with the addition of one or more heterologous peptides or heterologous polypeptides may be referred to herein as a fusion protein. Such fusion proteins are described herein and throughout.

In some embodiments, a heterologous peptide or heterologous polypeptide comprises a subcellular localization signal. In some embodiments, a subcellular localization signal can be a nuclear localization signal (NLS). In some embodiments, the NLS facilitates localization of a nucleic acid, protein, or small molecule to the nucleus, when present in a cell that contains a nuclear compartment. TABLE 2 lists exemplary NLS sequences. In some embodiments, the subcellular localization signal is a nuclear export signal (NES), a sequence to keep an effector protein retained in the cytoplasm, a mitochondrial localization signal for targeting to the mitochondria, a chloroplast localization signal for targeting to a chloroplast, an ER retention signal, and the like. In some embodiments, an effector protein described herein is not modified with a subcellular localization signal so that the polypeptide is not targeted to the nucleus, which can be advantageous depending on the circumstance (e.g., when the target nucleic acid is an RNA that is present in the cytosol).

In some embodiments, a heterologous peptide or heterologous polypeptide comprises a chloroplast transit peptide (CTP), also referred to as a chloroplast localization signal or a plastid transit peptide, which targets the effector protein to a chloroplast. Chromosomal transgenes from bacterial sources may require a sequence encoding a CTP sequence fused to a sequence encoding an expressed protein (e.g., the effector protein) if the expressed protein is to be compartmentalized in the plant plastid (e.g., chloroplast). The CTP may be removed in a processing step during translocation into the plastid. Accordingly, localization of an effector protein to a chloroplast is often accomplished by means of operably linking a polynucleotide sequence encoding a CTP sequence to the 5′ region of a polynucleotide encoding the exogenous protein.

In some embodiments, the heterologous polypeptide is an endosomal escape peptide (EEP). An EEP is an agent that quickly disrupts the endosome in order to minimize the amount of time that a delivered molecule, such an effector protein, spends in the endosome-like environment, and to avoid getting trapped in the endosomal vesicles and degraded in the lysosomal compartment. An exemplary EEP is set forth in TABLE 2.

In some embodiments, the heterologous polypeptide is a cell penetrating peptide (CPP), also known as a Protein Transduction Domain (PTD). A CPP or PTD is a polypeptide, polynucleotide, carbohydrate, or organic or inorganic compound that facilitates traversing a lipid bilayer, micelle, cell membrane, organelle membrane, or vesicle membrane.

Further suitable heterologous polypeptides include, but are not limited to, proteins (or fragments/domains thereof) that are boundary elements (e.g., CTCF), proteins and fragments thereof that provide periphery recruitment (e.g., Lamin A. Lamin B, etc.), and protein docking elements (e.g., FKBP/FRB. Pill/Abyl, etc.).

In some embodiments, a heterologous peptide or heterologous polypeptide comprises a protein tag. In some embodiments, the protein tag is referred to as purification tag or a fluorescent protein. The protein tag may be detectable for use in detection of the effector protein and/or purification of the effector protein. Accordingly, in some embodiments, compositions, systems and methods comprise a protein tag or use thereof. Any suitable protein tag may be used depending on the purpose of its use. Non-limiting examples of protein tags include a fluorescent protein, a histidine tag, e.g., a 6×His tag; a hemagglutinin (HA) tag; a FLAG tag; a Myc tag; and maltose binding protein (MBP). In some embodiments, the protein tag is a portion of MBP that can be detected and/or purified. Non-limiting examples of fluorescent proteins include green fluorescent protein (GFP), yellow fluorescent protein (YFP), red fluorescent protein (RFP), cyan fluorescent protein (CFP), mCherry, and tdTomato.

A heterologous polypeptide may be located at or near the amino terminus (N-terminus) of the effector protein disclosed herein. A heterologous polypeptide may be located at or near the carboxy terminus (C-terminus) of the effector proteins disclosed herein. In some embodiments, a heterologous polypeptide is located internally in an effector protein described herein (i.e., is not at the N- or C-terminus of an effector protein described herein) at a suitable insertion site.

In some embodiments, an effector protein described herein comprises 1, 2, 3, 4, 5, 6, 7, 8, 9, 10, or more heterologous polypeptides at or near the N-terminus, 1, 2, 3, 4, 5, 6, 7, 8, 9, 10, or more heterologous polypeptides at or near the C-terminus, or a combination of these (e.g., one or more heterologous polypeptides at the amino-terminus and one or more heterologous polypeptides at the carboxy terminus). When more than one heterologous polypeptide is present, each may be selected independently of the others, such that a single heterologous polypeptide may be present in more than one copy and/or in combination with one or more other heterologous polypeptides present in one or more copies. In some embodiments, a heterologous polypeptide is considered near the N- or C-terminus when the nearest amino acid of the heterologous polypeptide is within about 1, 2, 3, 4, 5, 10, 15, 20, 25, 30, 40, 50, or more amino acids along the polypeptide chain from the N- or C-terminus.

In some embodiments, a heterologous polypeptide described herein comprises a heterologous polypeptide sequence recited in TABLE 2. In some embodiments, effector proteins described herein comprise an amino acid sequence that is at least about 65%, at least about 70%, at least about 75%, at least about 80%, at least about 85%, at least about 90%, at least about 95%, at least about 97%, at least about 98%, at least about 99%, or about 100% identical to any one of the sequences recited in TABLE 1 and further comprises one or more of the sequences set forth in TABLE 2. In some embodiments, a heterologous peptide described herein may be a fusion partner as described en supra. TABLE 2.1 sets forth exemplary sequences of effector proteins described herein as modified with one or more heterologous proteins.

In some embodiments, effector proteins described herein are encoded by a codon optimized nucleic acid. In some embodiments, a nucleic acid sequence encoding an effector protein described herein, is codon optimized. In some embodiments, effector proteins described herein may be codon optimized for expression in a specific cell, for example, a bacterial cell, a plant cell, a eukaryotic cell, an animal cell, a mammalian cell, or a human cell. In some embodiments, the effector protein is codon optimized for a human cell.

In some embodiments, when describing a codon optimized nucleic acid, reference is made to a mutation of a nucleotide sequence encoding a polypeptide, such as a nucleotide sequence encoding an effector protein, to mimic the codon preferences of the intended host organism or cell while encoding the same polypeptide. Thus, the codons can be changed, but the encoded polypeptide remains unchanged. For example, if the intended target cell was a human cell, a human codon-optimized nucleotide sequence encoding an effector protein could be used. As another non-limiting example, if the intended host cell were a mouse cell, then a mouse codon-optimized nucleotide sequence encoding an effector protein could be generated. As another non-limiting example, if the intended host cell were a eukaryotic cell, then a eukaryote codon-optimized nucleotide sequence encoding an effector protein could be generated. As another non-limiting example, if the intended host cell were a prokaryotic cell, then a prokaryote codon-optimized nucleotide sequence encoding an effector protein could be generated. Codon usage tables are readily available, for example, at the “Codon Usage Database” available at www.kazusa.or.jp/codon

In some embodiments, effector proteins may comprise one or more modifications that may provide altered activity as compared to a naturally-occurring counterpart (e.g., a naturally-occurring nuclease or nickase activity which may be a naturally-occurring effector protein). In some embodiments, activity (e.g., nickase, nuclease, binding, activity) of effector proteins described herein can be measured relative to a naturally-occurring effector protein or compositions containing the same in a cleavage assay.

For example, effector proteins may comprise one or more modifications that may provide increased activity as compared to a naturally-occurring counterpart. As another example, effector proteins may provide increased catalytic activity (e.g., nickase, nuclease, binding activity) as compared to a naturally-occurring counterpart. Effector proteins may provide enhanced nucleic acid binding activity (e.g., enhanced binding of a guide nucleic acid, and/or target nucleic acid) as compared to a naturally-occurring counterpart. An effector protein may have a 1%, 5%, 10%, 20%, 30%, 40%, 50%, 60%, 70%, 80%, 90%, 100%, 120%, 140%, 160%, 180%, 200%, or more, increase of the activity of a naturally-occurring counterpart.

Engineered proteins may provide enhanced nuclease or nickase activity as compared to a naturally occurring nuclease or nickase. By way of non-limiting example, some engineered proteins exhibit optimal activity at lower salinity and viscosity than the protoplasm of their bacterial cell of origin. Also, by way of non-limiting example, bacteria often comprise protoplasmic salt concentrations greater than 250 mM and room temperature intracellular viscosities above 2 centipoise, whereas engineered proteins exhibit optimal activity (e.g., cis-cleavage activity) at salt concentrations below 150 mM and viscosities below 1.5 centipoise. The present disclosure leverages these dependencies by providing engineered proteins in solutions optimized for their activity and stability.

Alternatively, effector proteins may comprise one or more modifications that reduce the activity of the effector proteins relative to a naturally occurring nuclease, or nickase. An effector protein may have a 100%, 90%, 80%, 70%, 60%, 50%, 40%, 30%, 20%, 10%, 5%, 1%, or less, decrease of the activity of a naturally occurring counterpart. Decreased activity may be decreased catalytic activity (e.g., nickase, nuclease, binding activity) as compared to a naturally-occurring counterpart.

An engineered protein may comprise a modified form of a wild type counterpart protein (e.g., an effector protein). The modified form of the wild type counterpart may comprise an amino acid change (e.g., deletion, insertion, or substitution) that reduces the nucleic acid-cleaving activity of the effector protein relative to the wild type counterpart. For example, a nuclease domain (e.g., RuvC domain) of an effector protein may be deleted or mutated relative to a wild type counterpart effector protein so that it is no longer functional or comprises reduced nuclease activity. The modified form of the effector protein may have less than 90%, less than 80%, less than 70%, less than 60%, less than 50%, less than 40%, less than 30%, less than 20%, less than 10%, less than 5%, or less than 1% of the nucleic acid-cleaving activity of the wild-type counterpart.

Engineered proteins may have no substantial nucleic acid-cleaving activity. An effector protein that has decreased catalytic activity may be referred to as catalytically or enzymatically inactive, catalytically or enzymatically dead, as a dead protein or a dCas protein. Engineered proteins may be enzymatically inactive or “dead,” that is it may bind to a nucleic acid but not cleave it. An enzymatically inactive protein may comprise an enzymatically inactive domain (e.g, inactive nuclease domain). For example, a nuclease domain (e.g., RuvC domain) of an effector protein may be deleted or mutated relative to a wildtype counterpart so that it is no longer functional or comprises reduced nuclease activity.

Enzymatically inactive may refer to an activity less than 1%, less than 2%, less than 3%, less than 4%, less than 5%, less than 6%, less than 7%, less than 8%, less than 9%, or less than 10% activity compared to the wild-type counterpart. A dead protein may associate with a guide nucleic acid to activate or repress transcription of a target nucleic acid. In some embodiments, a catalytically inactive effector protein is fused to a fusion partner protein that confers an alternative activity to an effector protein activity. In some instances, the enzymatically inactive protein is fused with a protein comprising recombinase activity. Other such fusion proteins are described herein and throughout. Furthermore, nuclease-dead effector proteins are described further herein.

### Fusion Proteins

In some instances, an effector protein is a fusion protein, wherein the fusion protein comprises an effector protein and a fusion partner protein. In some instances, the effector protein comprises an amino acid sequence that is at least 65%, at least 70%, at least 75%, at least 80%, at least 85%, at least 90%, at least 95%, or 100% identical to any one of the sequences as set forth in TABLE 1. In some instances, the amino acid of the effector protein is at least 65%, at least 70%, at least 75%, at least 80%, at least 85%, at least 90%, at least 95%, or 100% identical to any one of the sequences as set forth in TABLE 1.

A fusion partner protein is also simply referred to herein as a fusion partner. In some embodiments, the fusion partner comprises a polypeptide or peptide that is fused or linked to the effector protein. In some embodiments, when describing fused sequences, reference is made to at least two sequences that are connected together, such as by a linker, or by conjugation (e.g., chemical conjugation or enzymatic conjugation). In some embodiments, the fusion partner is fused to the N-terminus of the effector protein. In some embodiments, the fusion partner protein is fused to the C-terminus of the effector protein. In some embodiments, the fusion protein is a heterologous peptide or polypeptide as described herein. In some embodiments, the fusion partner is not an effector protein as described herein. In some embodiments, the fusion partner comprises a second effector protein or a multimeric form thereof. Accordingly, in some embodiments, the fusion protein comprises more than one effector protein. In such embodiments, the fusion protein can comprise at least two effector proteins that are same. In some embodiments, the fusion protein comprises at least two effector proteins that are different. In some embodiments, the multimeric form is a homomeric form. In some embodiments, the multimeric form is a heteromeric form. Unless otherwise indicated, reference to effector proteins throughout the present disclosure include fusion proteins thereof.

In some embodiments, a fusion partner imparts some function or activity to a fusion protein that is not provided by an effector protein. Such activities may include but are not limited to nuclease activity, methyltransferase activity, demethylase activity. DNA repair activity. DNA damage activity, deamination activity, dismutase activity, alkylation activity, depurination activity, oxidation activity, dimer forming activity (e.g., pyrimidine dimer forming activity), integrase activity, transposase activity, recombinase activity, polymerase activity, ligase activity, helicase activity, photolyase activity, glycosylase activity, acetyltransferase activity, deacetylase activity, kinase activity, phosphatase activity, ubiquitin ligase activity, deubiquitinating activity, adenylation activity, deadenylation activity. SUMOylating activity, deSUMOylating activity, ribosylation activity, deribosylation activity, myristoylation activity or demyristoylation activity, modification of a polypeptide associated with target nucleic acid (e.g., a histone), and/or signaling activity.

In some embodiments, a fusion partner may provide signaling activity. In some embodiments, a fusion partner may inhibit or promote the formation of multimeric complex of an effector protein. In an additional example, the fusion partner may directly or indirectly edit a target nucleic acid. Edits can be of a nucleobase, nucleotide, or nucleotide sequence of a target nucleic acid. In some embodiments, the fusion partner may interact with additional proteins, or functional fragments thereof, to make modifications to a target nucleic acid. In other embodiments, the fusion partner may modify proteins associated with a target nucleic acid. In some embodiments, a fusion partner may modulate transcription (e.g., inhibits transcription, increases transcription) of a target nucleic acid. In yet another example, a fusion partner may directly or indirectly inhibit, reduce, activate or increase expression of a target nucleic acid.

### Multimeric Complex Formation Modification Activity

In some instances, the fusion partner promotes the formation of a multimeric complex of the effector protein. In some instances, the fusion partner inhibits the formation of a multimeric complex of the effector protein. By way of a non-limiting example, the fusion protein may comprise an effector protein and a fusion partner comprising a Calcineurin A tag, wherein the fusion protein dimerizes in the presence of Tacrolimus (FK506). Also, by way of non-limiting example, the fusion protein may comprise an effector protein and a SpyTag configured to dimerize or associate with another effector protein in a multimeric complex. Multimeric complex formation is further described herein.

### Nucleic Acid Modification Activity

In some instances, fusion partners provide enzymatic activity that modifies a target nucleic acid. Such enzymatic activities include, but are not limited to, nuclease activity, methyltransferase activity, demethylase activity. DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity, and glycosylase activity.

In some instances, fusion partners have enzymatic activity that modifies the target nucleic acid. The target nucleic acid may comprise or consist of a ssRNA, dsRNA, ssDNA, or a dsDNA. Examples of enzymatic activity that modifies the target nucleic acid include, but are not limited to: nuclease activity such as that provided by a restriction enzyme (e.g., FokI nuclease); methyltransferase activity such as that provided by a methyltransferase (e.g., HhaI DNA m5c-methyltransferase (M.HhaI), DNA methyltransferase 1 (DNMT1), DNA methyltransferase 3a (DNMT3a), DNA methyltransferase 3b (DNMT3b), METI, DRM3 (plants), ZMET2, CMT1, CMT2 (plants)); demethylase activity such as that provided by a demethylase (e.g., Ten-Eleven Translocation (TET) dioxygenase 1 (TET1CD), TET1, DME, DML1, DML2, ROS1); DNA repair activity; DNA damage (e.g., oxygenation) activity; deamination activity such as that provided by a deaminase (e.g., a cytosine deaminase enzyme such as rat APOBEC1); dismutase activity; alkylation activity; depurination activity; oxidation activity; pyrimidine dimer forming activity; integrase activity such as that provided by an integrase and/or resolvase (e.g., Gin invertase such as the hyperactive mutant of the Gin invertase, GinH106Y; human immunodeficiency virus type 1 integrase (IN); Tn3 resolvase); transposase activity, recombinase activity such as that provided by a recombinase (e.g., catalytic domain of Gin recombinase); as well as polymerase activity, ligase activity, helicase activity, photolyase activity, and glycosylase activity.

In some embodiments, fusion partners target a ssRNA, dsRNA, ssDNA, or a dsDNA. In some embodiments, fusion partners target ssRNA. Non-limiting examples of fusion partners for targeting ssRNA include, but are not limited to, splicing factors (e.g., RS domains); protein translation components (e.g., translation initiation, elongation, and/or release factors; e.g., cIF4G); RNA methylases; RNA editing enzymes (e.g., RNA deaminases, e.g., adenosine deaminase acting on RNA (ADAR), including A to I and/or C to U editing enzymes); helicases; and RNA-binding proteins.

It is understood that a fusion partner may include an entire protein, or in some embodiments, may include a fragment of the protein (e.g., a functional domain). In some embodiments, the functional domain binds or interacts with a nucleic acid, such as ssRNA, including intramolecular and/or intermolecular secondary structures thereof (e.g., hairpins, stem-loops, etc.). The functional domain may interact transiently or irreversibly, directly, or indirectly. In some embodiments, a functional domain comprises a region of one or more amino acids in a protein that is required for an activity of the protein, or the full extent of that activity, as measured in an in vitro assay. Activities include but are not limited to nucleic acid binding, nucleic acid editing, nucleic acid mutating, nucleic acid modifying, nucleic acid cleaving, protein binding or combinations thereof. The absence of the functional domain, including mutations of the functional domain, would abolish or reduce activity.

Accordingly, fusion partners may comprise a protein or domain thereof selected from: endonucleases (e.g., RNase III, the CRR22 DYW domain, Dicer, and PIN (PilT N-terminus); SMG5 and SMG6; domains responsible for stimulating RNA cleavage (e.g., CPSF. CstF. CFIm and CFIIm); exonucleases such as XRN-1 or Exonuclease T; deadenylases such as HNT3; protein domains responsible for nonsense mediated RNA decay (e.g., UPF1, UPF2, UPF3, UPF3b, RNP S1, Y14, DEK, REF2, and SRm160); protein domains responsible for stabilizing RNA (e.g., PABP); proteins and protein domains responsible for polyadenylation of RNA (e.g., PAP1, GLD-2, and Star-PAP); proteins and protein domains responsible for polyuridinylation of RNA (e.g., CI DI and terminal uridylate transferase); and other suitable domains that affect nucleic acid modifications.

In some embodiments, an effector protein is a fusion protein, wherein the effector protein is fused to a chromatin-modifying enzyme. In some embodiments, the fusion protein chemically modifies a target nucleic acid, for example by methylating, demethylating, or acetylating the target nucleic acid in a sequence specific or non-specific manner.

### Base Editors

In some embodiments, fusion partners edit a nucleobase of a target nucleic acid. Fusion proteins comprising such a fusion partner and an effector protein may be referred to as base editors. Such a fusion partner may be referred to as a base editing enzyme. In some embodiments, a base editor comprises a base editing enzyme variant that differs from a naturally occurring base editing enzyme, but it is understood that any reference to a base editing enzyme herein also refers to a base editing enzyme variant. In some embodiments, a base editor may be a fusion protein comprising a base editing enzyme fused or linked to an effector protein. In some embodiments, the amino terminus of the fusion partner protein is linked to the carboxy terminus of the effector protein by the linker. In some embodiments, the carboxy terminus of the fusion partner protein is linked to the amino terminus of the effector protein by the linker. The base editor may be functional when the effector protein is coupled to a guide nucleic acid. The base editor may be functional when the effector protein is coupled to a target nucleic acid. The guide nucleic acid imparts sequence specific activity to the base editor. By way of non-limiting example, the effector protein may comprise a catalytically inactive effector protein (e.g., a catalytically inactive variant of an effector protein described herein). Also, by way of non-limiting example, the base editing enzyme may comprise deaminase activity. In general, a base editor comprises a deaminase that when fused with a protein changes a nucleobase to a different nucleobase, e.g., cytosine to thymine or guanine to adenine. In some instances, the base editor comprises a deaminase. Additional base editors are described herein.

In some embodiments, base editors are capable of catalyzing editing (e.g., a chemical modification) of a nucleobase of a nucleic acid molecule, such as DNA or RNA (single stranded or double stranded). In some embodiments, a base editing enzyme, and therefore a base editor, is capable of converting an existing nucleobase to a different nucleobase, such as: an adenine (A) to guanine (G); cytosine (C) to thymine (T); cytosine (C) to guanine (G); uracil (U) to cytosine (C); guanine (G) to adenine (A); hydrolytic deamination of an adenine or adenosine, or methylation of cytosine (e.g., CpG. CpA, CpT or CpC). In some embodiments, base editors edit a nucleobase on a ssDNA. In some embodiments, base editors edit a nucleobase on both strands of dsDNA. In some embodiments, base editors edit a nucleobase of an RNA.

In some embodiments, a base editing enzyme itself may or may not bind to the nucleic acid molecule containing the nucleobase. In some embodiments, upon binding to its target locus in the target nucleic acid (e.g., a DNA molecule), base pairing between the guide nucleic acid and target strand leads to displacement of a small segment of ssDNA in an “R-loop”. In some embodiments. DNA bases within the R-loop are edited by the base editor having the deaminase enzyme activity. In some embodiments, base editors for improved efficiency in eukaryotic cells comprise a catalytically inactive effector protein that may generate a nick in the non-edited strand, inducing repair of the non-edited strand using the edited strand as a template.

In some embodiments, a base editing enzyme comprises a deaminase enzyme. Exemplary deaminases are described in US20210198330, WO2021041945. WO2021050571A1, and WO2020123887, all of which are incorporated herein by reference in their entirety. Exemplary deaminase domains are described WO 2018027078 and WO2017070632, and each are hereby incorporated in its entirety by reference. Also, additional exemplary deaminase domains are described in Komor et al., Nature, 533, 420-424 (2016); Gaudelli et al., Nature, 551, 464-471 (2017); Komor et al., Science Advances, 3: caao4774 (2017), and Rees et al., Nat Rev Genet. 2018 December; 19 (12): 770-788. doi: 10.1038/s41576-018-0059-1, which are hereby incorporated by reference in their entirety. In some embodiments, the deaminase functions as a monomer. In some embodiments, the deaminase functions as heterodimer with an additional protein. In some embodiments, base editors comprise a DNA glycosylase inhibitor (e.g., an uracil glycosylase inhibitor (UGI) or uracil N-glycosylase (UNG)). In some embodiments, the fusion partner is a deaminasc, e.g., ADAR1/2. ADAR-2. AID, or any functional variant thereof.

In some embodiments, a base editor is a cytosine base editor (CBE). In some embodiments, the CBE may convert a cytosine to a thymine. In some embodiments, a cytosine base editing enzyme may accept ssDNA as a substrate but may not be capable of cleaving dsDNA, as fused to a catalytically inactive effector protein. In some embodiments, when bound to its cognate DNA, the catalytically inactive effector protein of the CBE may perform local denaturation of the DNA duplex to generate an R-loop in which the DNA strand not paired with a guide nucleic acid exists as a disordered single-stranded bubble. In some embodiments, the catalytically inactive effector protein generated ssDNA R-loop may enable the CBE to perform efficient and localized cytosine deamination in vitro. In some embodiments, deamination activity is exhibited in a window of about 4 to about 10 base pairs. In some embodiments, fusion to the catalytically inactive effector protein presents a target site to the cytosine base editing enzyme in high effective molarity, which may enable the CBE to deaminate cytosines located in a variety of different sequence motifs, with differing efficacies. In some embodiments, the CBE is capable of mediating RNA-programmed deamination of target cytosines in vitro or in vivo. In some embodiments, the cytosine base editing enzyme is a cytidine deaminase. In some embodiments, the cytosine base editing enzyme is a cytosine base editing enzyme described by Koblan et al. (2018) Nature Biotechnology 36:848-846; Komor et al. (2016) Nature 533:420-424; Koblan et al. (2021) “Efficient C•G-to-G•C base editors developed using CRISPRi screens, target-library analysis, and machine learning.” Nature Biotechnology; Kurt et al. (2021) Nature Biotechnology 39:41-46; Zhao et al. (2021) Nature Biotechnology 39:35-40; and Chen et al. (2021) Nature Communications 12:1384, all incorporated herein by reference.

In some embodiments. CBEs comprise a uracil glycosylase inhibitor (UGI) or uracil N-glycosylase (UNG). In some embodiments, base excision repair (BER) of U•G in DNA is initiated by a UNG, which recognizes a U•G mismatch and cleaves the glyosidic bond between a uracil and a deoxyribose backbone of DNA. In some embodiments. BER results in the reversion of the U•G intermediate created by the first CBE back to a C•G base pair. In some embodiments, the UNG may be inhibited by fusion of a UGI. In some embodiments, the CBE comprises a UGI. In some embodiments.

a C-terminus of the CBE comprises the UGI. In some embodiments, the UGI is a small protein from bacteriophage PBS. In some embodiments, the UGI is a DNA mimic that potently inhibits both human and bacterial UNG. In some embodiments, the UGI inhibitor is any protein or polypeptide that inhibits UNG. In some embodiments, the CBE may mediate efficient base editing in bacterial cells and moderately efficient editing in mammalian cells, enabling conversion of a C•G base pair to a T·A base pair through a U•G intermediate. In some embodiments, the CBE is modified to increase base editing efficiency while editing more than one strand of DNA.

In some embodiments, a CBE nicks a non-edited DNA strand. In some embodiments, the non-edited DNA strand nicked by the CBE biases cellular repair of a U•G mismatch to favor a U•A outcome, elevating base editing efficiency. In some embodiments, a APOBEC1-nickase-UGI fusion efficiently edits in mammalian cells, while minimizing frequency of non-target indels. In some embodiments, base editors do not comprise a functional fragment of the base editing enzyme. In some embodiments, base editors do not comprise a function fragment of a UGI, where such a fragment may be capable of excising a uracil residue from DNA by cleaving an N-glycosidic bond.

In some embodiments, the fusion protein further comprises a non-protein uracil-DNA glycosylase inhibitor (npUGI). In some embodiments, the npUGI is selected from a group of small molecule inhibitors of uracil-DNA glycosylase (UDG), or a nucleic acid inhibitor of UDG. In some embodiments, the npUGI is a small molecule derived from uracil. Examples of small molecule non-protein uracil-DNA glycosylase inhibitors, fusion proteins, and Cas-CRISPR systems comprising base editing activity are described in WO2021087246, which is incorporated by reference in its entirety.

In some embodiments, a cytosine base editing enzyme, and therefore a cytosine base editor, is a cytidine deaminase. In some embodiments, the cytidine deaminase base editor is generated by ancestral sequence reconstruction as described in WO2019226953, which is hereby incorporated by reference in its entirety. Non-limiting exemplary cytidine deaminases suitable for use with effector proteins described herein include: APOBEC1, APOBEC2, APOBEC3C, APOBEC3D, APOBEC3F, APOBEC3G, APOBEC3H, APOBEC4, APOBEC3A, BE1 (APOBEC1-XTEN-dCas9), BE2 (APOBEC1-XTEN-dCas9-UGI), BE3 (APOBEC1-XTEN-dCas9 (A840H)-UGI), BE3-Gam, saBE3, saBE4-Gam, BE4, BE4-Gam, saBE4, and saBE4-Gam as described in WO2021163587, WO2021087246, WO2021062227, and WO2020123887, which are incorporated herein by reference in their entirety.

In some embodiments, a base editor is a cytosine to guanine base editor (CGBE). A CGBE may convert a cytosine to a guanine.

In some embodiments, a base editor is an adenine base editor (ABE). An ABE may convert an adenine to a guanine. In some embodiments, an ABE converts an A•T base pair to a G•C base pair. In some embodiments, the ABE converts a target A•T base pair to G•C in vivo or in vitro. In some embodiments. ABEs provided herein reverse spontaneous cytosine deamination, which has been linked to pathogenic point mutations. In some embodiments. ABEs provided herein enable correction of pathogenic SNPs (˜47% of disease-associated point mutations). In some embodiments, the adenine comprises exocyclic amine that has been deaminated (e.g., resulting in altering its basc pairing preferences). In some embodiments, deamination of adenosine yields inosine. In some embodiments, inosine exhibits the base-pairing preference of guanine in the context of a polymerase active site, although inosine in the third position of a tRNA anticodon is capable of pairing with A. U, or C in mRNA during translation. Non-limiting exemplary adenine base editing enzymes suitable for use with effector proteins described herein include: ABE8c, ABE8.20m, APOBEC3A. Anc APOBEC (a.k.a. AncBE4 Max), and BtAPOBEC2. Non-limiting exemplary ABEs suitable for use herein include: ABE7. ABE8.1m, ABE8.2m, ABE8.3m, ABE8.4m, ABE8.5m, ABE8.6m, ABE8.7m, ABE8.8m, ABE8.9m. ABE8.10m, ABE8.11m. ABE8.12m, ABE8.13m, ABE8.14m, ABE8.15m, ABE8.16m, ABE8.17m. ABE8.18m, ABE8.19m, ABE8.20m, ABE8.21m. ABE8.22m, ABE8.23m, ABE8.24m, ABE8.1d, ABE8.2d, ABE8.3d, ABE8.4d, ABE8.5d, ABE8.6d, ABE8.7d, ABE8.8d, ABE8.9d, ABE8.10d, ABE8.11d, ABE8.12d, ABE8.13d, ABE8.14d, ABE8.15d, ABE8.16d, ABE8.17d, ABE8.18d, ABE8.19d, ABE8.20d, ABE8.21d, ABE8.22d, ABE8.23d, and ABE8.24d. In some embodiments, the adenine base editing enzyme is an adenine base editing enzyme described in Chu et al., (2021) The CRISPR Journal 4:2:169-177, incorporated herein by reference. In some embodiments, the adenine deaminase is an adenine deaminase described by Koblan et al. (2018) Nature Biotechnology 36:848-846, incorporated herein by reference. In some embodiments, the adenine base editing enzyme is an adenine base editing enzyme described by Tran et al. (2020) Nature Communications 11:4871.

In some embodiments, an adenine base editing enzyme of an ABE is an adenosine deaminasc. Non-limiting exemplary adenosine base editors suitable for use herein include ABE9. In some embodiments, the ABE comprises an engineered adenosine deaminase enzyme capable of acting on ssDNA. The engineered adenosine deaminase enzyme may be an adenosine deaminase variant that differs from a naturally occurring deaminase. Relative to the naturally occurring deaminase, the adenosine deaminase variant may comprise one or more amino acid alteration, including a V82S alteration, a T166R alteration, a Y147T alteration, a Y147R alteration, a Q154S alteration, a Y123H alteration, a Q154R alteration, or a combination thereof.

In some embodiments, a base editor comprises a deaminase dimer. In some embodiments, the base editor further comprising a base editing enzyme and an adenine deaminase (e.g., TadA). In some embodiments, the adenosine deaminase is a TadA monomer (e.g., Tad*7.10, TadA*8 or TadA*9). In some embodiments, the adenosine deaminase is a TadA*8 variant (e.g., any one of TadA*8.1. TadA*8.2, TadA*8.3, TadA*8.4, TadA*8.5, TadA*8.6, TadA*8.7, TadA*8.8, TadA*8.9, TadA*8.10. TadA*8.11, TadA*8.12, TadA*8.13, TadA*8.14, TadA*8.15, TadA*8.16, TadA*8.17, TadA*8.18. TadA*8.19, TadA*8.20, TadA*8.21, TadA*8.22, TadA*8.23, or TadA*8.24 as described in WO2021163587 and WO2021050571, which are each hereby incorporated by reference in its entirety). In some embodiments, the base editor comprises a base editing enzyme fused to TadA by a linker (e.g., wherein the base editing enzyme is fused to TadA at N-terminus or C-terminus by a linker).

In some embodiments, a base editing enzyme is a deaminase dimer comprising an ABE. In some embodiments, the deaminase dimer comprises an adenosine deaminase. In some embodiments, the deaminase dimer comprises TadA fused to a suitable adenine base editing enzyme including an: ABE8c, ABE8.20m, APOBEC3A, Anc APOBEC (a.k.a. AncBE4 Max), BtAPOBEC2, and variants thereof. In some embodiments, the adenine base editing enzyme is fused to amino-terminus or the carboxy-terminus of TadA.

In some embodiments, RNA base editors comprise an adenosine deaminase. In some embodiments, ADAR proteins bind to RNAs and alter their sequence by changing an adenosine into an inosine. In some embodiments, RNA base editors comprise an effector protein that is activated by or binds RNA.

In some embodiments, base editors are used to treat a subject having or a subject suspected of having a disease related to a gene of interest. In some embodiments, base editors are useful for treating a disease or a disorder caused by a point mutation in a gene of interest. In some embodiments, compositions, systems, and methods described herein comprise a base editor and a guide nucleic acid, wherein the guide nucleic acid directs the base editor to a sequence in a target gene.

### Reverse Transcriptase (RT) Editing

In some embodiments, systems and methods comprise components or uses of an RT editing system to modify a target nucleic acid. RT editing may also be referred to as prime editing or precise nucleobase editing. In some embodiments, an RT editing system comprises an effector protein that is linked to a fusion partner that comprises an RT editing enzyme. In some embodiments, an RT editing enzyme comprises a polymerase. In some embodiments, an RT editing enzyme comprises a reverse transcriptase. A non-limiting example of a reverse transcriptase is an M-MLV RT enzyme and variants thereof having polymerase activity. In some embodiments, the M-MLV RT enzyme comprises at least one mutation selected from D200N, L603W, T330P, T306K, and W313F relative to wildtype M-MLV RT enzyme.

In some instances, systems and methods comprise an RT editing enzyme, wherein the RT editing enzyme is not fused or linked to the effector protein. In some instances, the RT editing enzyme comprises a recruiting moiety that recruits the RT editing enzyme to the target nucleic acid. By way of non-limiting example, the RT editing enzyme may comprise a peptide that binds an aptamer, wherein the aptamer is located on a guide RNA, template RNA, or combination thereof. Also, by way of non-limiting example, the RT editing enzyme may be linked to a protein that binds to (or is bound by) the effector protein or a protein linked/fused to the effector protein.

In some embodiments, an RT editing enzyme may require an RT editing guide RNA (pegRNA) to catalyze editing. Such a pegRNA may be capable of identifying a target nucleotide or target sequence in a target nucleic acid to be edited and encoding a new genetic information that replaces the target nucleotide or target sequence in the target nucleic acid. An RT editing enzyme may require a pegRNA and a guide RNA, such as a single guide RNA, to catalyze the editing. In some embodiments, the RT editing system comprises a template RNA comprising a primer binding sequence that hybridizes to a primer sequence of the dsDNA molecule that is formed when target nucleic acid is cleaved, and a template sequence that is complementary to at least a portion of the target sequence of the dsDNA molecule except for at least one nucleotide. In some embodiments, the template RNA is covalently linked to a guide RNA. In some instances, the guide RNA is a single guide RNA. In some embodiments, the template RNA is not covalently linked to a guide RNA. In some embodiments, at least a portion of the template RNA hybridizes to the target nucleic acid. In some embodiments, the target nucleic acid is a dsDNA molecule. In some embodiments, at least a portion of the template RNA hybridizes to a first strand of the target nucleic acid and at least a portion of the single guide RNA hybridizes to a second strand of the target nucleic acid. In some embodiments, the pegRNA comprises: a guide RNA comprising a second region that is bound by the effector protein, and a first region comprising a spacer sequence that is complementary to a target sequence of the dsDNA molecule; and a template RNA comprising a primer binding sequence that hybridizes to a primer sequence of the dsDNA molecule that is formed when target nucleic acid is cleaved, and a template sequence that is complementary to at least a portion of the target sequence of the dsDNA molecule with the exception of at least one nucleotide. In some embodiments, the at least one nucleotide is incorporated into the target nucleic acid by activity of the RT editing enzyme, thereby modifying the target nucleic acid. In some embodiments, the spacer sequence is complementary to the target sequence on a target strand of the dsDNA molecule. In some embodiments, the spacer sequence is complementary to the target sequence on a non-target strand of the dsDNA molecule. In some embodiments, the primer binding sequence hybridizes to a primer sequence on the non-target strand of the dsDNA molecule. In some embodiments, the primer binding sequence hybridizes to a primer sequence on the target strand of the dsDNA molecule. In some embodiments, the target strand is cleaved. In some embodiments, the non-target strand is cleaved.

### Protein Modification Activity

In some instances, a fusion partner provides enzymatic activity that modifies a protein (e.g., a histone) associated with a target nucleic acid. Such enzymatic activities include, but are not limited to, methyltransferase activity, demethylase activity, acetyltransferase activity, deacetylase activity, kinase activity, phosphatase activity, ubiquitin ligase activity, deubiquitinating activity, adenylation activity, deadenylation activity. SUMOylating activity, deSUMOylating activity, ribosylation activity, de-ribosy lation activity, myristovlation activity, and demyristovlation activity.

In some instances, the fusion partner has enzymatic activity that modifies a protein associated with a target nucleic acid. The protein may be a histone, an RNA binding protein, or a DNA binding protein. Examples of such protein modification activities include methyltransferase activity such as that provided by a histone methyltransferase (HMT) (e.g., suppressor of variegation 3-9 homolog 1 (SUV39H1, also known as KMTIA), cuchromatic histone lysine methyltransferase 2 (G9A, also known as KMTIC and EHMT2), SUV39H2, ESET/SETDB1, SETIA, SETIB, MLLI to 5, ASHI, SYMD2, NSD1, DOTIL, Pr-SET7/8, SUV4-20H1, EZH2, RIZ1); demethylase activity such as that provided by a histone demethylase (e.g., Lysine Demethylase 1A (KDMIA also known as LSD1), JHDM2a/b, JMJD2A/JHDM3A, JMJD2B, JMJD2C/GASCI, JMJD2D, JARIDIA/RBP2, JARIDIB/PLU-1, JARIDIC/SMCX, JARIDID/SMCY, UTX, JMJD3); acetyltransferase activity such as that provided by a histone acetylase transferase (e.g., catalytic core/fragment of the human acetyltransferase p300, GCN5, PCAF, CBP, TAF1, TIP60/PLIP, MOZ/MYST3, MORF/MYST4, HBO1/MYST2, HMOF/MYST1, SRC1, ACTR, P160, CLOCK); deacetylase activity such as that provided by a histone deacetylasc (e.g., HDAC1, HDAC2, HDAC3, HDAC8, HDAC4, HDAC5, HDAC7, HDAC9, SIRT1, SIRT2, HDAC11); kinase activity, phosphatase activity, ubiquitin ligase activity, deubiquitinating activity, adenylation activity, deadenylation activity. SUMOylating activity, deSUMOylating activity, ribosylation activity, deribosylation activity, myristoylation activity, and demyristoylation activity.

### CRISPRa Fusions and CRISPRi Fusions

In some instances, fusion partners include, but are not limited to, a protein that directly and/or indirectly provides for increased or decreased transcription and/or translation of a target nucleic acid (e.g., a transcription activator or a fragment thereof, a protein or fragment thereof that recruits a transcription activator, a small molecule/drug-responsive transcription and/or translation regulator, a translation-regulating protein, etc.). In some instances, fusion partners that increase or decrease transcription include a transcription activator domain or a transcription repressor domain, respectively.

In some instances, the fusion partner is a protein (or a domain from a protein) that increases transcription, also referred to as a transcription activator. In some embodiments, fusion proteins comprising the described fusion partners and an effector protein may be referred to as CRISPRa fusions. Transcriptional activators may promote transcription via recruitment of transcription activator proteins, modification of target DNA such as demethylation, recruitment of a DNA modifier, modulation of histones associated with target DNA, recruitment of a histone modifier such as those that modify acetylation and/or methylation of histones, or a combination thereof. In some instances, the fusion partner is a reverse transcriptase. In some embodiments, fusion partners increase expression of the target nucleic acid relative to its expression in the absence of the fusion effector protein. Relative expression, including transcription and RNA levels, may be assessed, quantified, and compared, e.g., by RT-qPCR. In some embodiments, fusion partners comprise a transcriptional activator.

Non-limiting examples of fusion partners that promote or increase transcription include, but are not limited to: transcriptional activators such as VP16. VP64. VP48. VP160, p65 subdomain (e.g., from NFkB), and activation domain of EDLL and/or TAL activation domain (e.g., for activity in plants); histone lysine methyltransferases such as SETIA, SETIB, MLLI to 5, ASHI, SYMD2, NSD1; histone lysine demethylases such as JHDM2a/b, UTX, JMJD3; histone acetyltransferases such as GCN5, PCAF, CBP, p300, TAF1, TIP60/PLIP, MOZ/MYST3, MORF/MYST4, SRC1, ACTR, P160, CLOCK; and DNA demethylases such as Ten-Eleven Translocation (TET) dioxygenase 1 (TET1CD), TET1, DME, DML1, DML2, and ROS1; and functional domains thereof. Other non-limiting examples of suitable fusion partners include: proteins and protein domains responsible for stimulating translation (e.g., Staufen); proteins and protein domains responsible for (e.g., capable of) modulating translation (e.g., translation factors such as initiation factors, elongation factors, release factors, etc., e.g., cIF4G); proteins and protein domains responsible for stimulation of RNA splicing (e.g., Serine/Arginine-rich (SR) domains); and proteins and protein domains responsible for stimulating transcription (e.g., CDK7 and HIV Tat).

In some embodiments, fusions partners inhibit or reduce expression of a target nucleic acid. In some embodiments, fusion proteins comprising described fusion partners and an effector protein may be referred to as CRISPRi fusions. In some embodiments, fusion partners reduce expression of the target nucleic acid relative to its expression in the absence of the fusion effector protein. Relative expression, including transcription and RNA levels, may be assessed, quantified, and compared, e.g., by RT-qPCR. In some embodiments, fusion partners may comprise a transcriptional repressor. In some embodiments, the transcriptional repressors may inhibit transcription by: recruitment of other transcription factor proteins; modification of target DNA such as methylation; recruitment of a DNA modifier; modulation of histones associated with target DNA; recruitment of a histone modifier such as those that modify acetylation and/or methylation of histones; or a combination thereof.

In some instances, the fusion partner modulates transcription (e.g., inhibits transcription, increases transcription) of a target nucleic acid. In some instances, the fusion partner is a protein (or a domain from a protein) that inhibits transcription, also referred to as a transcriptional repressor. Transcriptional repressors may inhibit transcription via recruitment of transcription inhibitor proteins, modification of target DNA such as methylation, recruitment of a DNA modifier, modulation of histones associated with target DNA, recruitment of a histone modifier such as those that modify acetylation and/or methylation of histones, or a combination thereof.

Non-limiting examples of fusion partners that decrease or inhibit transcription include, but are not limited to: transcriptional repressors such as the Krüppel associated box (KRAB or SKD); KOX1 repression domain; the Mad mSIN3 interaction domain (SID); the ERF repressor domain (ERD), the SRDX repression domain (e.g., for repression in plants); histone lysine methyltransferases such as Pr-SET7/8, SUV4-20H1, RIZ1, and the like; histone lysine demethylases such as JMJD2A/JHDM3A, JMJD2B, JMJD2C/GASCI, JMJD2D, JARIDIA/RBP2, JARIDIB/PLU-1, JARIDIC/SMCX, JARIDID/SMCY; histone lysine deacetylases such as HDAC1, HDAC2, HDAC3, HDAC8, HDAC4, HDAC5, HDAC7, HDAC9, SIRT1, SIRT2, HDAC11; DNA methylases such as HhaI DNA m5c-methyltransferase (M.HhaI), DNA methyltransferase 1 (DNMT1), DNA methyltransferase 3a (DNMT3a), DNA methyltransferase 3b (DNMT3b), METI, DRM3 (plants), ZMET2, CMT1, CMT2 (plants); and periphery recruitment elements such as Lamin A, and Lamin B; and functional domains thereof. Other non-limiting examples of suitable fusion partners include: proteins and protein domains responsible for repressing translation (e.g., Ago2 and Ago4); proteins and protein domains responsible for repression of RNA splicing (e.g., PTB, Sam68, and hnRNP A1); proteins and protein domains responsible for reducing the efficiency of transcription (e.g., FUS (TLS)).

In some instances, fusion proteins are targeted by a guide nucleic acid (guide RNA) to a specific location in the target nucleic acid and exert locus-specific regulation such as blocking RNA polymerase binding to a promoter (which selectively inhibits transcription activator function), and/or modifying the local chromatin status (e.g., when a fusion sequence is used that modifies the target nucleic acid or modifies a protein associated with the target nucleic acid). In some instances, the modifications are transient (e.g., transcription repression or activation). In some instances, the modifications are inheritable. For instance, epigenetic modifications made to a target nucleic acid, or to proteins associated with the target nucleic acid, e.g., nucleosomal histones, in a cell, are observed in cells produced by proliferation of the cell.

In some instances, the fusion partner comprises an RNA splicing factor. The RNA splicing factor may be used (in whole or as fragments thereof) for modular organization, with separate sequence-specific RNA binding modules and splicing effector domains. Non-limiting examples of RNA splicing factors include members of the Serine/Arginine-rich (SR) protein family contain N-terminal RNA recognition motifs (RRMs) that bind to exonic splicing enhancers (ESEs) in pre-mRNAs and C-terminal RS domains that promote exon inclusion. As another example, the hnRNP protein hnRNP A1 binds to cxonic splicing silencers (ESSs) through its RRM domains and inhibits exon inclusion through a C-terminal Glycine-rich domain. Some splicing factors may regulate alternative use of splice site (ss) by binding to regulatory sequences between the two alternative sites. For example ASF/SF2 may recognize ESEs and promote the use of intron proximal sites, whereas hnRNP A1 may bind to ESSs and shift splicing towards the use of intron distal sites. One application for such factors is to generate ESFs that modulate alternative splicing of endogenous genes, particularly disease associated genes. For example. Bcl-x pre-mRNA produces two splicing isoforms with two alternative 5′ splice sites to encode proteins of opposite functions. The long splicing isoform Bcl-xL is a potent apoptosis inhibitor expressed in long-lived postmitotic cells and is up-regulated in many cancer cells, protecting cells against apoptotic signals. The short isoform Bcl-xS is a pro-apoptotic isoform and expressed at high levels in cells with a high turnover rate (e.g., developing lymphocytes). The ratio of the two Bel-x splicing isoforms is regulated by multiple co-elements that are located in either the core exon region or the exon extension region (i.e., between the two alternative 5′ splice sites). For more examples, see WO2010075303, which is hereby incorporated by reference in its entirety.

### Recombinases

In some embodiments, fusion partners comprise a recombinase. In some embodiments, effector proteins described herein are fused with the recombinase. In some embodiments, the effector proteins have reduced nuclease activity or no nuclease activity. In some embodiments, the recombinase is a site-specific recombinase.

In some embodiments, a catalytically inactive effector protein is fused with a recombinase, wherein the recombinase can be a site-specific recombinase. Such polypeptides can be used for site-directed transgene insertion. In some embodiments, when describing a transgene, reference is made to a nucleotide sequence that is inserted into a cell for expression of said nucleotide sequence in the cell. A transgene is meant to include (1) a nucleotide sequence that is not naturally found in the cell (e.g., a heterologous nucleotide sequence); (2) a nucleotide sequence that is a mutant form of a nucleotide sequence naturally found in the cell into which it has been introduced; (3) a nucleotide sequence that serves to add additional copies of the same (e.g., exogenous or homologous) or a similar nucleotide sequence naturally occurring in the cell into which it has been introduced; or (4) a silent naturally occurring or homologous nucleotide sequence whose expression is induced in the cell into which it has been introduced. A donor nucleic acid can comprise a transgene. The cell in which transgene expression occurs can be a target cell, such as a host cell

Non-limiting examples of site-specific recombinases include a tyrosine recombinase (e.g., Cre, Flp or lambda integrase), a serine recombinase (e.g., gamma-delta resolvase, Tn3 resolvase, Sin resolvase, Gin invertase, Hin invertase, Tn5044 resolvase, IS607 transposase and integrase), or mutants or variants thereof. In some embodiments, the recombinase is a serine recombinase. Non-limiting examples of serine recombinases include gamma-delta resolvase, Tn3 resolvase, Sin resolvase, Gin invertase, Hin invertase, Tn5044 resolvase, IS607 transposase, and IS607 integrase. In some embodiments, the site-specific recombinase is an integrase. Non-limiting examples of integrases include: Bxb1, wBeta, BL3, phiR4, A118, TG1, MR11, phi370, SPBc, TP901-1, phiRV, FC1, K38, phiBT1, and phiC31. Further discussion and examples of suitable recombinase fusion partners are described in U.S. Pat. No. 10,975,392, which is incorporated herein by reference in its entirety. In some embodiments, the fusion protein comprises a linker that links the recombinase to the Cas-CRISPR domain of the effector protein. In some embodiments, the linker is The-Ser.

### Additional Fusion Partners

In some instances, the fusion partner is a chloroplast transit peptide (CTP), also referred to as a plastid transit peptide. In some instances, this targets the fusion protein to a chloroplast. Chromosomal transgenes from bacterial sources must have a sequence encoding a CTP sequence fused to a sequence encoding an expressed protein if the expressed protein is to be compartmentalized in the plant plastid (e.g. chloroplast). The CTP is removed in a processing step during translocation into the plastid. Accordingly, localization of an exogenous protein to a chloroplast is often accomplished by means of operably linking a polynucleotide sequence encoding a CTP sequence to the 5′ region of a polynucleotide encoding the exogenous protein. In some instances, the CTP is located at the N-terminus of the fusion protein. Processing efficiency may, however, be affected by the amino acid sequence of the CTP and nearby sequences at the amino terminus (NH2 terminus) of the peptide.

In some instances, the fusion partner is an endosomal escape peptide. Exemplary endosomal escape peptides are set forth in TABLE 2.

Further suitable fusion partners include, but are not limited to, proteins (or fragments/domains thereof) that are boundary elements (e.g., CTCF), proteins and fragments thereof that provide periphery recruitment (e.g., Lamin A. Lamin B, etc.), protein docking elements (e.g., FKBP/FRB, Pill/Abyl, etc.).

### Linkers for Peptides

In some embodiments, a linker comprises a bond or molecule that links a first polypeptide to a second polypeptide. Accordingly, in some embodiments, effector proteins and fusion partners of a fusion effector protein are connected by a linker. The linker may comprise or consist of a covalent bond. The linker may comprise or consist of a chemical group. In some embodiments, the linker comprises an amino acid. In some embodiments, a peptide linker comprises at least two amino acids linked by an amide bond. In general, the linker connects a terminus of the effector protein to a terminus of the fusion partner. In some embodiments, carboxy terminus of the effector protein is linked to the amino terminus of the fusion partner. In some embodiments, carboxy terminus of the fusion partner is linked to the amino terminus of the effector protein. In some embodiments, the effector protein and the fusion partner are directly linked by a covalent bond.

In some embodiments, linkers comprise one or more amino acids. In some embodiments, linker is a protein. In some instances, a terminus of the effector protein is linked to a terminus of the fusion partner through an amide bond. In some embodiments, a terminus of the effector protein is linked to a terminus of the fusion partner through a peptide bond. In some embodiments, linkers comprise an amino acid. In some embodiments, linkers comprise a peptide. In some instances, an effector protein is coupled to a fusion partner via a linker protein. The linker protein may have any of a variety of amino acid sequences. A linker protein may comprise a region of rigidity (e.g., beta sheet, alpha helix), a region of flexibility, or any combination thereof. In some instances, the linker comprises small amino acids, such as glycine and alanine, that impart high degrees of flexibility. The ordinarily skilled artisan will recognize that design of a peptide conjugated to any desired element may include linkers that are all or partially flexible, such that the linker may include a flexible linker as well as one or more portions that confer less flexible structure. Suitable linkers include proteins of 4 linked amino acids to 40 linked amino acids in length, or between 4 linked amino acids and 25 linked amino acids in length. In some embodiments, linked amino acids described herein comprise at least two amino acids linked by an amide bond. In some embodiments, the linker is from 1 to 100 amino acids in length. In some embodiments, the linker is more 100 amino acids in length. In some embodiments, the linker is from 10 to 27 amino acids in length.

These linkers may be produced by using synthetic, linker-encoding oligonucleotides to couple the proteins, or may be encoded by a nucleic acid sequence encoding a fusion protein (e.g., an effector protein coupled to a fusion partner). Examples of linker proteins include glycine polymers (G) n, glycine-serine polymers (including, for example (GS) n. GSGGSn (SEQ ID NO: 1480), GGSGGSn (SEQ ID NO: 1481), and GGGSn (SEQ ID NO: 1482), where n is an integer of at least one), glycine-alanine polymers, and alanine-serine polymers. Exemplary linkers may comprise amino acid sequences including, but not limited to GS, GSGGS (SEQ ID NO: 1483), GGSGGS (SEQ ID NO: 1484), GGGS (SEQ ID NO: 1485), GGSG (SEQ ID NO: 1486), GGSGG (SEQ ID NO: 1487), GSGSG (SEQ ID NO: 1488), GSGGG (SEQ ID NO: 1489), GGGSG (SEQ ID NO: 1490), and GSSSG (SEQ ID NO: 1491).

In some embodiments, the linker comprises one or more repeats a tri-peptide GGS. In some embodiments, the linker is an XTEN linker. In some embodiments, the XTEN linker is an XTEN80 linker. In some embodiments, the XTEN linker is an XTEN20 linker. In some embodiments, the XTEN20 linker has an amino acid sequence of GSGGSPAGSPTSTEEGTSESATPGSG (SEQ ID NO: 1633).

In some embodiments, linkers do not comprise an amino acid. In some embodiments, linkers do not comprise a peptide. In some embodiments, linkers comprise a nucleotide, a polynucleotide, a polymer, or a lipid. In some embodiments, linker may be a polyethylene glycol (PEG), polypropylene glycol (PPG), co-poly(ethylene/propylene) glycol, polyoxyethylene (POE), polyurethane, polyphosphazene, polysaccharides, dextran, polyvinyl alcohol, polyvinylpyrrolidones, polyvinyl ethyl ether, polyacrylamide, polyacrylate, polycyanoacrylates, lipid polymers, chitins, hyaluronic acid, heparin, or an alkyl linker.

TABLE 2.1 sets forth exemplary sequences of effector proteins described herein as modified with one or more linkers.

### Nuclease-Dead Effector Proteins

In some instances, the effector protein can comprise an enzymatically inactive and/or “dead” (abbreviated by “d”) effector protein in combination (e.g., fusion) with a polypeptide comprising recombinase activity. Although an effector protein normally has nuclease activity, in some instances, an effector protein does not have nuclease activity. In some instances, an effector protein comprising at least 60%, at least 65%, at least 70%, at least 75%, at least 80%, at least 85%, at least 90%, at least 92%, at least 95%, at least 97%, at least 98%, at least 99%, or 100% sequence identity with the sequence of TABLE 1 is a nuclease-dead effector protein. In some instances, the effector protein comprising at least 60%, at least 65%, at least 70%, at least 75%, at least 80%, at least 85%, at least 90%, at least 92%, at least 95%, at least 97%, at least 98%, at least 99%, or 100% sequence identity with the sequence of TABLE 1 is modified or engineered to be a nuclease-dead effector protein.

The effector protein can comprise a modified form of a wild type counterpart. The modified form of the wild type counterpart can comprise an amino acid change (e.g., deletion, insertion, or substitution) that reduces the nucleic acid-cleaving activity of the effector protein. For example, a nuclease domain (e.g., HEPN domain) of an effector polypeptide can be deleted or mutated so that it is no longer functional or comprises reduced nuclease activity. The modified form of the effector protein can have less than 90%, less than 80%, less than 70%, less than 60%, less than 50%, less than 40%, less than 30%, less than 20%, less than 10%, less than 5%, or less than 1% of the nucleic acid-cleaving activity of the wild-type counterpart. The modified form of an effector protein can have no substantial nucleic acid-cleaving activity. When an effector protein is a modified form that has no substantial nucleic acid-cleaving activity, it can be referred to as enzymatically inactive and/or dead. A dead effector polypeptide can bind to a target sequence but may not cleave the target nucleic acid. A dead effector polypeptide can associate with a guide nucleic acid to activate or repress transcription of a target nucleic acid.

### Multimeric Complexes

Compositions, systems, and methods of the present disclosure may comprise a multimeric complex or uses thereof, wherein the multimeric complex comprises multiple effector proteins that non-covalently interact with one another. A multimeric complex may comprise enhanced activity relative to the activity of any one of its effector proteins alone. For example, a multimeric complex comprising two effector proteins may comprise greater nucleic acid binding affinity, cis-cleavage activity, and/or transcollateral cleavage activity than that of either of the effector proteins provided in monomeric form. A multimeric complex may have an affinity for a target region of a target nucleic acid and is capable of catalytic activity (e.g., cleaving, nicking or modifying the nucleic acid) at or near the target region. Multimeric complexes may be activated when complexed with a guide nucleic acid. Multimeric complexes may be activated when complexed with a guide nucleic acid and a target nucleic acid. In some instances, the multimeric complex cleaves the target nucleic acid. In some instances, the multimeric complex nicks the target nucleic acid.

Various aspects of the present disclosure include compositions and methods comprising multiple effector proteins, and uses thereof, respectively. An effector protein comprising at least 70% sequence identity to any one of the sequences of TABLE 1 may be provided with a second effector protein. Two effector proteins may target different nucleic acid sequences. Two effector proteins may target different types of nucleic acids (e.g., a first effector protein may target double- and single-stranded nucleic acids, and a second effector protein may only target single-stranded nucleic acids).

In some instances, multimeric complexes comprise at least one effector protein, or a fusion protein thereof, comprising an amino acid sequence with at least 65%, at least 70%, at least 75%, at least 80%, at least 85%, at least 90%, at least 95% or 100% identity to any one of the sequences of TABLE 1. In some instances, multimeric complexes comprise at least one effector protein or a fusion protein thereof, wherein the amino acid sequence of the effector protein is at least 65%, at least 70%, at least 75%, at least 80%, at least 85%, at least 90%, at least 95% or 100% identical to any one of the sequences of TABLE 1.

In some instances, the multimeric complex is a dimer comprising two effector proteins of identical amino acid sequences. In some instances, the multimeric complex comprises a first effector protein and a second effector protein, wherein the amino acid sequence of the first effector protein is at least 90%, at least 92%, at least 94%, at least 96%, at least 98% identical, or at least 99% identical to the amino acid sequence of the second effector protein.

In some instances, the multimeric complex is a heterodimeric complex comprising at least two effector proteins of different amino acid sequences. In some instances, the multimeric complex is a heterodimeric complex comprising a first effector protein and a second effector protein, wherein the amino acid sequence of the first effector protein is less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, or less than 10% identical to the amino acid sequence of the second effector protein.

In some instances, a multimeric complex comprises at least two effector proteins. In some instances, a multimeric complex comprises more than two effector proteins. In some instances, a multimeric complex comprises two, three or four effector proteins. In some instances, at least one effector protein of the multimeric complex comprises an amino acid sequence with at least 65%, at least 70%, at least 75%, at least 80%, at least 85%, at least 90%, at least 95% or 100% identity to any one of the sequences of TABLE 1. In some instances, each effector protein of the multimeric complex comprises an amino acid sequence with at least 65%, at least 70%, at least 75%, at least 80%, at least 85%, at least 90%, at least 95% or 100% identity to any one of the sequences of TABLE 1.

### Synthesis, Isolation and Assaying

Effector proteins of the present disclosure may be synthesized, using any suitable method. In some embodiments, the effector proteins may be produced in vitro or by eukaryotic cells or by prokaryotic cells. In some embodiments, the effector proteins may be further processed by unfolding (e.g, heat denaturation, dithiothreitol reduction, etc.) and may be further refolded, using any suitable method.

Any suitable method of generating and assaying the effector proteins described herein may be used. Such methods include, but are not limited to, site-directed mutagenesis, random mutagenesis, combinatorial libraries, and other mutagenesis methods described herein (scc, e.g., Sambrook et al., Molecular Cloning: A Laboratory Manual. Third Ed., Cold Spring Harbor Laboratory. New York (2001); Ausubel et al., Current Protocols in Molecular Biology. John Wiley and Sons. Baltimore, MD (1999); Gillman et al., Directed Evolution Library Creation; Methods and Protocols (Methods in Molecular Biology) Springer, 2nd ed (2014)). One non-limiting example of a method for preparing an effector protein is to express recombinant nucleic acids encoding the effector protein in a suitable microbial organism, such as a bacterial cell, a yeast cell, or other suitable cell, using methods well known in the art. Exemplary methods are also described in the Examples provided herein.

In some embodiments, an effector protein provided herein is an isolated effector protein. In some embodiments, the effector proteins may be isolated and purified for use in compositions, systems, and/or methods described herein. In some embodiments, methods described here may include the step of isolating effector proteins described herein. Any suitable method to provide isolated effector proteins described herein may be used in the present disclosure, for example, recombinant expression systems, precipitation, gel filtration, ion-exchange, reverse-phase and affinity chromatography, and the like. Other well-known methods are described in Deutscher et al., Guide to Protein Purification; Methods in Enzymology. Vol. 182. (Academic Press. (1990)). Alternatively, the isolated polypeptides of the present disclosure can be obtained using well-known recombinant methods (scc, e.g., Sambrook et al., Molecular Cloning: A Laboratory Manual. Third Ed., Cold Spring Harbor Laboratory. New York (2001); and Ausubel et al., Current Protocols in Molecular Biology. John Wiley and Sons. Baltimore. MD (1999)). The methods and conditions for biochemical purification of a polypeptide described herein can be chosen by those skilled in the art, and purification monitored, for example, by a functional assay.

In some embodiments, compositions, systems, and methods described herein may further comprise a purification tag that can be attached to an effector protein, or a nucleic acid encoding the purification tag that can be attached to a nucleic acid encoding the effector protein as described herein. In some embodiments, the purification tag may be an amino acid sequence which can attach or bind with high affinity to a separation substrate and assist in isolating the protein of interest from its environment, which may be its biological source, such as a cell lysate. Attachment of the purification tag may be at the N or C terminus of the effector protein. Furthermore, an amino acid sequence recognized by a protease or a nucleic acid encoding for an amino acid sequence recognized by a protease, such as TEV protease or the HRV3C protease may be inserted between the purification tag and the effector protein, such that biochemical cleavage of the sequence with the protease after initial purification liberates the purification tag. Purification and/or isolation may be performed through high performance liquid chromatography (HPLC), exclusion chromatography, gel electrophoresis, affinity chromatography, or other purification technique. Non-limiting examples of purification tags are as described herein.

In some embodiments, effector proteins described herein are isolated from cell lysate. In some embodiments, the compositions described herein may comprise 20% or more by weight, 75% or more by weight, 95% or more by weight, or 99.5% or more by weight of an effector protein, related to the method of preparation of compositions described herein and its purification thereof, wherein percentages may be upon total protein content in relation to contaminants. Thus, in some embodiments, the effector protein is at least 80% pure, at least 85% pure, at least 90% pure, at least 95% pure, at least 98% pure, or at least 99% pure (e.g., free of contaminants, non-engineered proteins or other macromolecules, etc.).

### PAMs

Effector proteins of the present disclosure may cleave or nick a target nucleic acid within or near a protospacer adjacent motif (PAM) sequence of the target nucleic acid. In some embodiments, the target nucleic acid is a double stranded nucleic acid comprising a target strand and a non-target strand. In some embodiments, cleavage occurs within 1, 2, 3, 4, 5, 6, 7, 8, 9, 10, 15, 20, 25, 30, 35, 40, 45, or 50 nucleotides of a 5′ or 3′ terminus of a PAM sequence. In some embodiments, effector proteins described herein recognize a PAM sequence. In some embodiments, recognizing a PAM sequence comprises interacting with a sequence adjacent to the PAM. In some embodiments, a target nucleic acid comprises a target sequence that is adjacent to a PAM sequence. In some embodiments, the effector protein does not require a PAM to bind and/or cleave a target nucleic acid.

In some embodiments, a target nucleic acid is a single stranded target nucleic acid comprising a target sequence. Accordingly, in some embodiments, the single stranded target nucleic acid comprises a PAM sequence described herein that is adjacent (e.g., within 1, 2, 3, 4, 5, 6, 7, 8, 9, 10, 15, 20, 25, 30, 35, 40, 45, or 50 nucleotides) or directly adjacent to the target sequence. In some embodiments, an RNP cleaves the single stranded target nucleic acid.

In some embodiments, a target nucleic acid is a double stranded nucleic acid comprising a target strand and a non-target strand, wherein the target strand comprises a target sequence. In some embodiments, the PAM sequence is located on the target strand. In some embodiments, the PAM sequence is located on the non-target strand. In some embodiments, the PAM sequence described herein is adjacent (e.g., within 1, 2, 3, 4, 5, 6, 7, 8, 9, 10, 15, 20, 25, 30, 35, 40, 45, or 50 nucleotides) to the target sequence on the target strand or the non-target strand. In some embodiments, such a PAM described herein is directly adjacent to the target sequence on the target strand or the non-target strand. In some embodiments, an RNP cleaves the target strand or the non-target strand. In some embodiments, the RNP cleaves both, the target strand and the non-target strand. In some embodiments, an RNP recognizes the PAM sequence, and hybridizes to a target sequence of the target nucleic acid. In some embodiments, the RNP cleaves the target nucleic acid, wherein the RNP has recognized the PAM sequence and is hybridized to the target sequence. A target nucleic acid may comprise a PAM sequence adjacent to a sequence that is complementary to a guide nucleic acid spacer region. In some embodiments, a target nucleic acid described herein comprises any one of the PAM sequences set forth in TABLE 3.

In some embodiments, an effector protein described herein, or a multimeric complex thereof, recognizes a PAM on a target nucleic acid. In some embodiments, multiple effector proteins of the multimeric complex recognize a PAM on a target nucleic acid. In some embodiments, at least two of the multiple effector proteins recognize the same PAM sequence. In some embodiments, at least two of the multiple effector proteins recognize different PAM sequences. In some embodiments, only one effector protein of the multimeric complex recognizes a PAM on a target nucleic acid. In some instances, the effector protein recognizes a PAM sequence as shown in TABLE 3. In some instances, the effector protein recognizes a PAM sequence comprising any of the following nucleotide sequences as set forth in TABLE 3. In some instances, a composition comprising an effector protein recognizes a PAM sequence comprising any of the following nucleotide sequences as set forth in TABLE 3.

Effector proteins of the present disclosure, dimers thereof, and multimeric complexes thereof may cleave or nick a target nucleic acid within or near a protospacer adjacent motif (PAM) sequence of the target nucleic acid. In some instances, cleavage occurs within 1, 2, 3, 4, 5, 6, 7, 8, 9 or 10 nucleotides of a 5′ or 3′ terminus of a PAM sequence.

In some embodiments, a PAM sequence provided herein comprises any one of the nucleotide sequences recited in TABLE 3. PAMs used in compositions, systems, and methods herein are further described throughout the application.

### II. Nucleic Acid Systems

### Guide Nucleic Acids

The compositions, systems, and methods of the present disclosure may comprise a guide nucleic acid or a use thereof. Unless otherwise indicated, compositions, systems and methods comprising guide nucleic acids or uses thereof, as described herein and throughout, include DNA molecules, such as expression vectors, that encode a guide nucleic acid.

In some embodiments, when describing a nucleic acid, reference is made to a polymer of nucleotides. In some embodiments, a nucleic acid may comprise ribonucleotides, deoxyribonucleotides, combinations thereof, and modified versions of the same. In some embodiments, a nucleic acid may be single-stranded or double-stranded, unless specified. Non-limiting examples of nucleic acids are double stranded DNA (dsDNA), single stranded (ssDNA), messenger RNA, genomic DNA, cDNA, DNA-RNA hybrids, and a polymer comprising purine and pyrimidine bases or other natural, chemically or biochemically modified, non-natural, or derivatized nucleotide bases. In some embodiments, nucleic acids as described herein may comprise one or more mutations, one or more engineered modifications, or both.

In some embodiments, when describing nucleotide(s) and/or nucleoside(s), in the context of a nucleic acid molecule having multiple residues, reference is made to the sugar and base of the residue contained in the nucleic acid molecule. Similarly, a skilled artisan could understand that linked nucleotides and/or linked nucleosides, as used in the context of a nucleic acid having multiple linked residues, are interchangeable and describe linked sugars and bases of residues contained in a nucleic acid molecule. In some embodiments, when describing nucleobase(s) or linked nucleobase, as used in the context of a nucleic acid molecule, it can be understood as describing the base of the residue contained in the nucleic acid molecule, for example, the base of a nucleotide, nucleosides, or linked nucleotides or linked nucleosides. A person of ordinary skill in the art when referring to nucleotides, nucleosides, and/or nucleobases would also understand the differences between RNA and DNA (generally the exchange of uridine for thymidine or vice versa) and the presence of nucleoside analogs, such as modified uridines, do not contribute to differences in identity or complementarity among polynucleotides as long as the relevant nucleotides (such as thymidine, uridine, or modified uridine) have the same complement (e.g., adenosine for all of thymidine, uridine, or modified uridine; another example is cytosine and 5-methylcytosine, both of which have guanosine or modified guanosine as a complement). Thus, for example, the sequence 5′-AXG where X is any modified uridine, such as pseudouridine. NI-methyl pseudouridine, or 5-methoxyuridine, is considered 100% identical to AUG in that both are perfectly complementary to the same sequence (5′-CAU).

In general, a guide nucleic acid is a nucleic acid molecule that binds to an effector protein, thereby forming a ribonucleoprotein complex (RNP). Guide nucleic acids, when complexed with an effector protein, may bring the effector protein into proximity of a target nucleic acid. Sufficient conditions for hybridization of a guide nucleic acid to a target nucleic acid and/or for binding of a guide nucleic acid to an effector protein include in vivo physiological conditions of a desired cell type or in vitro conditions sufficient for assaying catalytic activity of a protein, polypeptide or peptide described herein, such as the nuclease activity of an effector protein.

Guide nucleic acids, and any components thereof (e.g., spacer sequence, repeat sequence, linker nucleotide sequence, handle sequence, intermediary sequence etc.) may comprise DNA, RNA, or a combination thereof (e.g., RNA with a thymine base). Guide nucleic acids may include a chemically modified nucleobase or phosphate backbone. Guide nucleic acids may be referred to herein as a guide RNA (gRNA). However, a guide RNA is not limited to ribonucleotides, but may comprise deoxyribonucleotides and other chemically modified nucleotides. A guide nucleic acid may comprise a naturally occurring guide nucleic acid. A guide nucleic acid may comprise a non-naturally occurring guide nucleic acid, including a guide nucleic acid that is designed to contain a chemical or biochemical modification. A guide nucleic acid of the present disclosure comprises one or more of the following: a) a single nucleic acid molecule; b) a DNA base; c) an RNA base; d) a modified base; c) a modified sugar; f) a modified backbone; and the like. Modifications are described herein and throughout the present disclosure (e.g., in the section entitled “Engineered Modifications”).

Guide nucleic acids are often referred to as “guide RNA.” However, a guide nucleic acid may comprise deoxyribonucleotides. The term “guide RNA.” as well as crRNA, includes guide nucleic acids comprising DNA bases and RNA bases. The guide RNA may be chemically synthesized or recombinantly produced. Guide nucleic acids and portions thereof may be found in or identified from a CRISPR array present in the genome of a host organism or cell. The sequence of the guide nucleic acid, or a portion thereof, may be different from the sequence of a naturally occurring nucleic acid.

In some embodiments, the guide nucleic acid comprises a nucleotide sequence that is at least 80%, at least 85%, at least 90%, at least 95%, at least 98%, at least 99%, or 100% complementary to the target sequence. In some embodiments, the guide nucleic acid comprises at least 10 contiguous nucleotides that are complementary to the target sequence in the target nucleic acid. In some embodiments, guide nucleic acid comprises a spacer sequence that is at least 80%, at least 85%, at least 90%, at least 95%, at least 98%, at least 99%, or 100% complementary to the target sequence.

The guide nucleic acid may comprise a first region complementary to a target nucleic acid (FR1) and a second region that is not complementary to the target nucleic acid (FR2). In some embodiments. FR1 is located 5′ to FR2 (FR1-FR2). In some embodiments. FR2 is located 5′ to FR1 (FR2-FR1). In some embodiments, the FR2 comprises one or more repeat sequences, handle sequence, or intermediary sequence. In some embodiments, an effector protein binds to at least a portion of the FR2. In some embodiments, the FR1 comprises a spacer sequence, wherein the spacer sequence can interact in a sequence-specific manner with (e.g., has complementarity with, or can hybridize to a target sequence in) a target nucleic acid.

In some embodiments, the first region, the second region, or both may be about 8 nucleic acids, about 10 nucleic acids, about 12 nucleic acids, about 14 nucleic acids, about 16 nucleic acids, about 18 nucleic acids, about 20 nucleic acids, about 22 nucleic acids, about 24 nucleic acids, about 26 nucleic acids, about 28 nucleic acids, about 30 nucleic acids, about 32 nucleic acids, about 34 nucleic acids, about 36 nucleic acids, about 38 nucleic acids, about 40 nucleic acids, about 42 nucleic acids, about 44 nucleic acids, about 46 nucleic acids, about 48 nucleic acids, or about 50 nucleic acids long.

In some embodiments, the first region, the second region, or both may be from about 8 to about 12, from about 8 to about 16, from about 8 to about 20, from about 8 to about 24, from about 8 to about 28, from about 8 to about 30, from about 8 to about 32, from about 8 to about 34, from about 8 to about 36, from about 8 to about 38, from about 8 to about 40, from about 8 to about 42, from about 8 to about 44, from about 8 to about 48, or from about 8 to about 50 nucleic acids long.

In some embodiments, the first region, the second region, or both may comprise a GC content of about 1%, about 5%, about 10%, about 15%, about 20%, about 25%, about 30%, about 40%, about 50%, about 60%, about 70%, about 80%, about 90%, about 95%, or about 99%. In some embodiments, the first region, the second region, or both may comprise a GC content of from about 1% to about 95%, from about 5% to about 90%, from about 10% to about 80%, from about 15% to about 70%, from about 20% to about 60%, from about 25% to about 50%, or from about 30% to about 40%.

In some embodiments, the first region, the second region, or both may have a melting temperature of about 38° C., about 40° C., about 42° C., about 44° C., about 46° C., about 48° C., about 50° C., about 52° C., about 54° C., about 56° C., about 58° C., about 60° C., about 62° C., about 64° C., about 66° C., about 68° C., about 70° C., about 72° C., about 74° C., about 76° C., about 78° C., about 80° C., about 82° C., about 84° C., about 86° C., about 88° C., about 90° C., or about 92° C. In some embodiments, the first region, the second region, or both may have a melting temperature of from about 35° C., to about 40° C., from about 35° C., to about 45° C., from about 35° C., to about 50° C., from about 35° C., to about 55° C., from about 35° C., to about 60° C., from about 35° C., to about 65° C., from about 35° C., to about 70° C., from about 35° C., to about 75° C., from about 35° C., to about 80° C., or from about 35° C., to about 85° C.

In some embodiments, the compositions, systems, devices, kits, and methods of the present disclosure further comprise an additional nucleic acid, wherein a portion of the additional nucleic acid at least partially hybridizes to the first region of the guide nucleic acid. In some embodiments, the additional nucleic acid is at least partially hybridized to the 5′ end of the second region of the guide nucleic acid. In some embodiments, an unhybridized portion of the additional nucleic acid, at least partially, interacts with an effector protein or polypeptide. In some embodiments, the compositions, systems, devices, kits, and methods of the present disclosure comprise a dual nucleic acid system comprising the guide nucleic acid and the additional nucleic acid as described herein.

The guide nucleic acid may also form complexes as described through herein. For example, a guide nucleic acid may hybridize to another nucleic acid, such as target nucleic acid, or a portion thereof. In another example, a guide nucleic acid may complex with an effector protein. In such embodiments, a guide nucleic acid-effector protein complex may be described herein as an RNP. In some embodiments, when in a complex, at least a portion of the complex may bind, recognize, and/or hybridize to a target nucleic acid. For example, when a guide nucleic acid and an effector protein are complexed to form an RNP, at least a portion of the guide nucleic acid hybridizes to a target sequence in a target nucleic acid. Those skilled in the art in reading the below specific examples of guide nucleic acids as used in RNPs described herein, will understand that in some embodiments, a RNP may hybridize to one or more target sequences in a target nucleic acid, thereby allowing the RNP to modify and/or recognize a target nucleic acid or sequence contained therein (e.g., PAM) or to modify and/or recognize non-target sequences depending on the guide nucleic acid, and in some embodiments, the effector protein, used. In some embodiments, effector proteins are targeted by a guide nucleic acid (e.g., a guide RNA) to a specific location in the target nucleic acid where they exert locus-specific regulation. Non-limiting examples of locus-specific regulation include blocking RNA polymerase binding to a promoter (which selectively inhibits transcription activator function), and/or modifying local chromatin (e.g., modifying the target nucleic acid or modifying a protein associated with the target nucleic acid). The guide RNA may bind to a target nucleic acid (e.g., a single strand of a target nucleic acid) or a portion thereof, an amplicon thereof, or a portion thereof. By way of non-limiting example, a guide nucleic acid may bind to a target nucleic acid, such as DNA or RNA, from a gene associated with a genetic disorder, or an amplicon thereof, as described herein. In some embodiments, a guide nucleic acid may hybridize, at least partially, to a target nucleic acid wherein the target nucleic acid comprises one or more mutations, for example as described herein. In some embodiments, a guide nucleic acid may hybridize to a target nucleic acid comprising a mutation, wherein the guide nucleic acid hybridizes upstream or downstream of said mutation. In some embodiments, a guide nucleic acid of an RNP complex may hybridize to a target nucleic acid comprising a mutation, whereby the target nucleic acid is modified and wherein upon modification of the target nucleic acid, the wild-type target nucleic acid is restored. In some instances, the mutation may be a non-wild-type reading frame wherein modification of the target nucleic acid restores the wild-type reading frame. Modification of target nucleic acids by RNP complexes are described throughout herein.

In some embodiments, a guide nucleic acid may comprise or form intramolecular secondary structure (e.g., hairpins, stem-loops, etc.). In some embodiments, a guide nucleic acid comprises a stem-loop structure comprising a stem region and a loop region. In some embodiments, the stem region is 4 to 8 linked nucleotides in length. In some embodiments, the stem region is 5 to 6 linked nucleotides in length. In some embodiments, the stem region is 4 to 5 linked nucleotides in length. In some embodiments, when describing sequence length or linked units, reference is made to a nucleic acid (polynucleotide) or polypeptide, may be expressed as “kilobases” (kb) or “base pairs (bp).”. Thus, a length of 1 kb refers to a length of 1000 linked nucleosides, and a length of 500 bp refers to a length of 500 linked nucleosides. Similarly, a protein having a length of 500 linked amino acids may also be simply described as having a length of 500 amino acids

In some embodiments, the guide nucleic acid comprises a pseudoknot (e.g., a secondary structure comprising a stem, at least partially, hybridized to a second stem or half-stem secondary structure). An effector protein may recognize a guide nucleic acid comprising multiple stem regions. In some embodiments, the nucleotide sequences of the multiple stem regions are identical to one another. In some embodiments, the nucleotide sequences of at least one of the multiple stem regions is not identical to those of the others. In some embodiments, the guide nucleic acid comprises at least 2, at least 3, at least 4, or at least 5 stem regions.

In some embodiments, the compositions, systems, and methods of the present disclosure may comprise an additional guide nucleic acid or a use thereof. In some embodiments, the compositions, systems, and methods of the present disclosure comprise two or more guide nucleic acids (e.g., 2, 3, 4, 5, 6, 7, 9, 10 or more guide nucleic acids), and/or uses thereof. An additional guide nucleic acid or multiple additional guide nucleic acids can target an effector protein to a different location in the target nucleic acid by binding to a different portion of the target nucleic acid from the first guide nucleic acid. For example, a guide nucleic acid can bind a portion of the target nucleic acid that is upstream of a premature stop codon of a targeted gene that is formed as a result of an out-of-frame genetic mutation in a cell or subject as described herein (e.g., the dystrophin gene), wherein the additional guide nucleic acid can bind to a portion of the target nucleic acid that is located either upstream or downstream of where the first guide RNA has targeted. In such embodiments, the dual-guided compositions, systems, and methods described herein can modify the target nucleic acid in two locations. In some embodiments, the dual-guided compositions, systems, and methods described herein can cleave the target nucleic acid in the two locations targeted by the guide RNAs. In certain embodiments, upon removal of the sequence between the guide nucleic acids, the wild-type reading frame is restored resulting in at least a partially functional protein. In certain embodiments, upon removal of the sequence between the target sequences of the guide nucleic acids, any desired genomic sequences such as an entire exon or a region of sequences involved in mRNA splicing or multiple exons or certain specific sequences can be deleted. In some embodiments, a donor nucleic acid is inserted in replacement of the deleted sequence. The modification of the target nucleic acid at two different loci is referred to herein as “dual-cutting”. Accordingly, in some embodiments, dual-guide nucleic acid compositions, systems, and methods can comprise two effector proteins, individually corresponding a guide RNA or a single effector protein with two different guide RNA to achieve dual-cutting.

In some embodiments, compositions, systems and methods described herein comprise the use of a guide nucleic acid and an additional guide nucleic acid wherein the guide nucleic acid hybridizes to a first loci of the target nucleic acid, and wherein the additional guide nucleic acid hybridizes to a second loci of the target nucleic acid, and wherein the target nucleic acid comprises a mutation. In some embodiments, a guide nucleic acid may hybridize to a first loci of a target nucleic acid comprising a mutation that is upstream or downstream of a mutation associated with disease or disorder, and the additional guide nucleic acid may hybridize to a second loci of the target nucleic acid that is upstream or downstream of a mutation associated a disease or disorder. In some embodiments, a guide nucleic acid may hybridize to a first loci of a target nucleic acid comprising a mutation that is upstream or downstream of a mutation associated with disease or disorder, and the additional guide nucleic acid may hybridize to a second loci of the target nucleic acid that is upstream or downstream of a mutation associated a disease or disorder such that guide nucleic acid and the additional guide nucleic acid hybridizes to either side of said mutation. In some embodiments, modification of such a target nucleic acid results in the modification of the nucleic acid (such as a nucleotide sequence) comprising the mutation, and may result in, for example, deletion of the nucleic acid comprising the mutation and/or located between the guide nucleic acid and the additional guide nucleic acid, substitution or insertion of the same.

In some embodiments where two locations (loci) of a target nucleic acid are targeted, the first loci and the second loci of the target nucleic acid may be located at least 1, at least 10, at least 20, at least 30, at least 40, at least 50, at least 60, at least 70, at least 80, at least 90 or at least 100 nucleotides apart. In some embodiments, the first loci and the second loci of the target nucleic acid may be located between 100 and 200, 200 and 300, 300 and 400, 400 and 500, 500 and 600, 600 and 700, 700 and 800, 800 and 900 or 900 and 1000 nucleotides apart. In some embodiments, the first loci and/or the second loci of the target nucleic acid are located in an intron of a gene. In some embodiments, the first loci and/or the second loci of the target nucleic acid are located in an exon of a gene. In some embodiments, the first loci and/or the second loci of the target nucleic acid span an exon-intron junction of a gene. In some embodiments, the first portion and/or the second portion of the target nucleic acid are located on either side of an exon and cutting at both sites results in deletion of the exon. In some embodiments, composition, systems, and methods comprise a donor nucleic acid that may be inserted in replacement of a deleted or cleaved sequence of the target nucleic acid. In some embodiments, compositions, systems, and methods comprising multiple guide nucleic acids or uses thereof comprise multiple effector proteins, wherein the effector proteins may be identical, non-identical, or combinations thereof.

In some embodiments, the guide nucleic acid comprises 10, 11, 12, 13, 14, 15, 16, 17, 18, 19, 20, 21, 22, 23, 24, 25, 26, 27, 28, 29, or 30 linked nucleosides. In general, a guide nucleic acid comprises at least linked nucleosides. In some embodiments, a guide nucleic acid comprises at least 25 linked nucleosides. A guide nucleic acid may comprise 10 to 50 linked nucleosides. In some embodiments, the guide nucleic acid comprises or consists essentially of about 12 to about 80 linked nucleosides, about 12 to about 50, about 12 to about 45, about 12 to about 40, about 12 to about 35, about 12 to about 30, about 12 to about 25, from about 12 to about 20, about 12 to about 19, about 19 to about 20, about 19 to about 25, about 19 to about 30, about 19 to about 35, about 19 to about 40, about 19 to about 45, about 19 to about 50, about 19 to about 60, about 20 to about 25, about 20 to about 30, about 20 to about 35, about 20 to about 40, about 20 to about 45, about 20 to about 50, or about 20 to about 60 linked nucleosides. In some embodiments, the guide nucleic acid has about 10 to about 60, about 20 to about 50, or about 30 to about 40 linked nucleosides.

In some embodiments, a guide nucleic acid comprises about: 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 linked nucleotides. In general, a guide nucleic acid comprises at least: 10, 15, 20, 25, 30, 35, 40, 45, 50, 55, 60 linked nucleotides. In some embodiments, the guide nucleic acid has about 10 to about 60, about 20 to about 50, or about 30 to about 40 linked nucleotides.

In some embodiments, a guide nucleic acid comprises at least 10, 11, 12, 13, 14, 15, 16, 17, 18, 19, 20, 21, 22, 23, 24, 25, 26, 27, 28, 29, or 30 contiguous nucleotides that are complementary to a eukaryotic sequence. Such a eukaryotic sequence is a nucleotide sequence that is present in a host eukaryotic cell. Such a nucleotide sequence is distinguished from nucleotide sequences present in other host cells, such as prokaryotic cells, or viruses. Said sequences present in a eukaryotic cell can be located in a gene, an exon, an intron, a non-coding (e.g., promoter or enhancer) region, a selectable marker, tag, signal, and the like. In some embodiments, a target sequence is a eukaryotic sequence.

In some embodiments, a length of a guide nucleic acid is about 30 to about 120 linked nucleotides. In some embodiments, the length of a guide nucleic acid is about 40 to about 100, about 40 to about 90, about 40 to about 80, about 40 to about 70, about 40 to about 60, about 40 to about 50, about 50 to about 90, about 50 to about 80, about 50 to about 70, or about 50 to about 60 linked nucleotides. In some embodiments, the length of a guide nucleic acid is about 40, about 45, about 50, about 55, about 60, about 65, about 70 or about 75 linked nucleotides. In some embodiments, the length of a guide nucleic acid is greater than about 15, about 20, about 25, about 30, about 35, about 40, about 45, about 50, about 55, about 60, about 65, about 70 or about 75 linked nucleotides. In some embodiments, the length of a guide nucleic acid is not greater than about 40, about 45, about 50, about 55, about 60, about 65, about 70, about 75, about 80, about 85, about 90, about 95, about 100, about 105, about 110, about 115, about 120, or about 125 linked nucleotides.

In some embodiments, guide nucleic acids comprise additional elements that contribute additional functionality (e.g., stability, heat resistance, etc.) to the guide nucleic acid. Such elements may be one or more nucleotide alterations, nucleotide sequences, intermolecular secondary structures, or intramolecular secondary structures (e.g., one or more hair pin regions, one or more bulges, etc.).

In some embodiments, guide nucleic acids comprise one or more linkers connecting different nucleotide sequences as described herein. A linker may comprise 1, 2, 3, 4, 5, 6, 7, 8, 9, 10, or more nucleotides. A linker may be any suitable linker, examples of which are described herein.

In some embodiments, the guide nucleic acid comprises a nucleotide sequence as described herein (e.g., TABLE 4, TABLE 5, TABLE 5.1, TABLE 6, TABLE 7, or TABLE 8). Such nucleotide sequences described herein (e.g., TABLE 4, TABLE 5, TABLE 5.1, TABLE 6, TABLE 7, or TABLE 8) may be described as a nucleotide sequence of either DNA or RNA, however, no matter the form the sequence is described, it is readily understood that such nucleotide sequences can be revised to be RNA or DNA, as needed, for describing a sequence within a guide nucleic acid itself or the sequence that encodes a guide nucleic acid, such as a nucleotide sequence described herein for a vector. Similarly, disclosure of the nucleotide sequences described herein (e.g., TABLE 4, TABLE 5, TABLE 5.1, TABLE 6, TABLE 7, or TABLE 8) also discloses the complementary nucleotide sequence, the reverse nucleotide sequence, and the reverse complement nucleotide sequence, any one of which can be a nucleotide sequence for use in a guide nucleic acid as described herein.

In some embodiments, the guide nucleic acid comprises a sequence that is at least 70%, at least 80%, at least 85%, at least 90%, at least 95%, or 100% identical to any one of the sequences set forth in TABLE 4, TABLE 5, TABLE 5.1, TABLE 6, TABLE 7, or TABLE 8, or any combination thereof.

In some embodiments, the guide nucleic acid comprises a spacer sequence and/or a repeat sequence. In some embodiments, the guide nucleic acid comprises a spacer sequence that is at least 70%, at least 75%, at least 80%, at least 85%, at least 90%, at least 95%, or 100% identical to any one of the sequences of TABLE 4 and a repeat sequence that is at least 70%, at least 75%, at least 80%, at least 85%, at least 90%, at least 95%, or 100% identical to any one of the sequences of TABLE 5.

In some embodiments, the guide nucleic acid comprises a spacer sequence and/or a handle sequence. In some embodiments, the guide nucleic acid comprises a spacer sequence that is at least 70%, at least 75%, at least 80%, at least 85%, at least 90%, at least 95%, or 100% identical to any one of the sequences of TABLE 4 and a handle sequence that is at least 70%, at least 75%, at least 80%, at least 85%, at least 90%, at least 95%, or 100% identical to any one of the sequences of TABLE 6.

In some embodiments, the guide nucleic acid comprises a crRNA sequence. In some embodiments, the guide nucleic acid comprises a crRNA sequence that is at least 70%, at least 75%, at least 80%, at least 85%, at least 90%, at least 95%, or 100% identical to any one of the sequences of TABLE 7.

In some embodiments, the guide nucleic acid comprises a sgRNA sequence. In some embodiments, the guide nucleic acid comprises a sgRNA sequence that is at least 70%, at least 75%, at least 80%, at least 85%, at least 90%, at least 95%, or 100% identical to any one of the sequences of TABLE 8.

### Spacer Sequence

In some embodiments, guide nucleic acids described herein may comprise one or more spacer sequences. The spacer region may comprise complementarity with (e.g., hybridize to) a target sequence of a target nucleic acid. In some embodiments, a spacer sequence is capable of hybridizing to a target sequence of a target nucleic acid. In some embodiments, a spacer sequence comprises a nucleotide sequence that is, at least partially, hybridizable to an equal length of a sequence (e.g., a target sequence) of a target nucleic acid.

Exemplary hybridization conditions are described herein. In some embodiments, when describing a sequence that can hybridize or is hybridizable, reference is made to a nucleotide sequence that is able to noncovalently interact, i.e, form Watson-Crick base pairs and/or G/U base pairs, or anncal, to another nucleotide sequence in a sequence-specific, antiparallel, manner (i.e., a nucleotide sequence specifically interacts to a complementary nucleotide sequence) under the appropriate in vitro and/or in vivo conditions of temperature and solution ionic strength. Standard Watson-Crick base-pairing includes: adenine (A) pairing with thymidine (T), adenine (A) pairing with uracil (U), and guanine (G) pairing with cytosine (C) for both DNA and RNA. In addition, for hybridization between two RNA molecules (e.g., dsRNA), and for hybridization of a DNA molecule with an RNA molecule (e.g., when a DNA target nucleic acid base pairs with a guide RNA, etc.): guanine (G) can also base pair with uracil (U). For example G/U base-pairing is at least partially responsible for the degeneracy (i.e., redundancy) of the genetic code in the context of tRNA anti-codon base-pairing with codons in mRNA. Thus, a guanine (G) can be considered complementary to both an uracil (U) and to an adenine (A). Accordingly, when a G/U base-pair can be made at a given nucleotide position, the position is not considered to be non-complementary, but is instead considered to be complementary. While hybridization typically occurs between two nucleotide sequences that are complementary, mismatches between bases are possible. It is understood that two nucleotide sequences need not be 100% complementary to be specifically hybridizable, hybridizable, partially hybridizable, or for hybridization to occur. Moreover, a nucleotide sequence may hybridize over one or more segments such that intervening or adjacent segments are not involved in the hybridization event (e.g., a bulge, a loop structure or hairpin structure, etc.). The conditions appropriate for hybridization between two nucleotide sequences depend on the length of the sequence and the degree of complementarity, variables which are well known in the art. For hybridizations between nucleic acids with short stretches of complementarity (e.g, complementarity over 35 or less, 30 or less, 25 or less, 22 or less, 20 or less, or 18 or less nucleotides) the position of mismatches may become important (see Sambrook et al., supra, 11.7-11.8). Typically, the length for a hybridizable nucleic acid is 8 nucleotides or more (e.g., 10 nucleotides or more, 12 nucleotides or more, 15 nucleotides or more, 20 nucleotides or more, 22 nucleotides or more, 25 nucleotides or more, or 30 nucleotides or more). Any suitable in vitro assay may be utilized to assess whether two sequences “hybridize”. One such assay is a melting point analysis where the greater the degree of complementarity between two nucleotide sequences, the greater the value of the melting temperature (Tm) for hybrids of nucleic acids having those sequences. The conditions of temperature and ionic strength determine the “stringency” of the hybridization. Temperature, wash solution salt concentration, and other conditions may be adjusted as necessary according to factors such as length of the region of complementation and the degree of complementation. Hybridization and washing conditions are well known and exemplified in Sambrook. J., Fritsch. E. F, and Maniatis. T. Molecular Cloning: A Laboratory Manual. Second Edition. Cold Spring Harbor Laboratory Press. Cold Spring Harbor (1989), particularly Chapter 11 and Table 11.1 therein; and Sambrook, J, and Russell. W., Molecular Cloning: A Laboratory Manual. Third Edition. Cold Spring Harbor Laboratory Press. Cold Spring Harbor (2001).

In some embodiments, the spacer sequence may function to direct an RNP complex comprising the guide nucleic acid to the target nucleic acid for detection and/or modification. The spacer sequence may function to direct a RNP to the target nucleic acid for detection and/or modification. A spacer sequence may be complementary to a target sequence that is adjacent to a PAM that is recognizable by an effector protein described herein.

In some embodiments, a spacer sequence comprises at least 5 to about 50 contiguous nucleotides that are complementary to a target sequence in a target nucleic acid. In some embodiments, a spacer sequence comprises at least 5 to about 50 linked nucleotides. In some embodiments, a spacer sequence comprises at least 5 to about 50, at least 5 to about 25, at least about 10 to at least about 25, or at least about 15 to about 25 linked nucleotides. In some embodiments, the spacer region is 15-28 linked nucleosides in length. In some embodiments, the spacer region is 15-26, 15-24, 15-22, 15-20, 15-18, 16-28, 16-26, 16-24, 16-22, 16-20, 16-18, 17-26, 17-24, 17-22, 17-20, 17-18, 18-26, 18-24, or 18-22 linked nucleosides in length. In some embodiments, the spacer region is 18-24 linked nucleosides in length. In some embodiments, the spacer region is at least 15 linked nucleosides in length. In some embodiments, the spacer region is at least 16, 18, 20, or 22 linked nucleosides in length. In some embodiments, the spacer region comprises at least 10, 11, 12, 13, 14, 15, 16, 17, 18, 19, 20, 21, 22, 23, 24, 25, 26, 27, 28, 29, or 30 nucleotides. In some embodiments, the spacer region is at least 17 linked nucleosides in length. In some embodiments, the spacer region is at least 18 linked nucleosides in length. In some embodiments, the spacer region is at least 20 linked nucleosides in length.

TABLE 4 provides illustrative spacer sequences for use with the compositions and methods of the disclosure. In some embodiments, the spacer sequence comprises at least 70%, at least 80%, at least 90%, at least 92%, at least 95%, at least 97%, or at least 99%, or 100% sequence identity to a sequence as set forth in TABLE 4. In some embodiments, systems and methods comprise a guide nucleic acid, wherein the guide nucleic acid comprises at least 12, at least 13, at least 14, at least 15, at least 16, at least 17, or at least 18 contiguous nucleobases of a sequence provided in TABLE 4. In some embodiments, systems and methods comprise a guide nucleic acid, wherein the guide nucleic acid comprises at least 12, at least 13, at least 14, at least 15, at least 16, at least 17, or at least 18 contiguous nucleobases of a sequence provided in TABLE 4 with the exception of not more than 1 or 2 nucleotides. In some embodiments, the guide nucleic acid comprises at least 12, at least 13, at least 14, at least 15, at least 16, at least 17, or at least 18 of the 5′ most contiguous nucleobases of a sequence provided in TABLE 4. In some embodiments, the guide nucleic acid comprises at least 12, at least 13, at least 14, at least 15, at least 16, at least 17, or at least 18 of the 3′ most contiguous nucleobases of a sequence provided in TABLE 4. In some embodiments, the guide nucleic acid comprises a sufficient number of contiguous nucleobases of TABLE 4 to convey hybridization to the target nucleic acid under physiological conditions. In some embodiments, the spacer sequence comprises one or more nucleobase alterations at one or more positions in any one of the sequences of TABLE 4. Alternative nucleobases can be any one or more of A. C. G. T or U, or a deletion, or an insertion.

In some embodiments, the spacer region is at least 80%, at least 85%, at least 90%, at least 95% or 100% complementary to a target sequence of the target nucleic acid. In some embodiments, the spacer region is 100% complementary to the target sequence of the target nucleic acid. In some embodiments, the spacer region comprises at least 15 contiguous nucleobases that are complementary to the target nucleic acid.

In some embodiments, spacer sequence is capable of hybridizing to an equal length portion of a target nucleic acid (e.g., a target sequence). In some embodiments, a target nucleic acid, such as DNA or RNA, may be a gene associated with a genetic disorder, or an amplicon thereof, as described herein. In some embodiments, a target nucleic acid is a gene selected from TABLE 9. In some embodiments, a spacer sequence comprises a sequence that is at least 65%, at least 70%, at least 75%, at least 80%, at least 85%, at least 90%, at least 95% or 100% complementary to a target sequence of a target nucleic acid selected from TABLE 9. In some embodiments, a target nucleic acid is a nucleic acid associated with a disease or syndrome set forth in TABLE 10. In some embodiments, a spacer sequence comprises a sequence that is at least 65%, at least 70%, at least 75%, at least 80%, at least 85%, at least 90%, at least 95% or 100% complementary to a target sequence of a target nucleic acid associated with a disease or syndrome set forth in TABLE 10. In some embodiments, the spacer sequence comprises at least 10, 11, 12, 13, 14, 15, 16, 17, 18, 19, 20, 21, 22, 23, 24, 25, 26, 27, 28, 29, or 30 contiguous nucleotides that are capable of hybridizing to the target sequence. In some embodiments, the spacer sequence comprises at least 10, 11, 12, 13, 14, 15, 16, 17, 18, 19, 20, 21, 22, 23, 24, 25, 26, 27, 28, 29, or 30 contiguous nucleotides that are complementary to the target sequence.

It is understood that the sequence of a spacer region need not be 100% complementary to that of a target sequence of a target nucleic acid to hybridize or hybridize specifically to the target sequence. The guide nucleic acid may comprise at least one uracil between nucleic acid residues 5 to 20 of the spacer region that is not complementary to the corresponding nucleoside of the target sequence. The guide nucleic acid may comprise at least one uracil between nucleic acid residues 5 to 9, 10 to 14, or 15 to 20 of the spacer region that is not complementary to the corresponding nucleoside of the target sequence. In some embodiments, the region of the target nucleic acid that is complementary to the spacer region comprises an epigenetic modification or a post-transcriptional modification. In some embodiments, the epigenetic modification comprises an acetylation, methylation, or thiol modification.

In some embodiments, a spacer sequence is adjacent to a repeat sequence. In some embodiments, a spacer sequence follows a repeat sequence in a 5′ to 3′ direction. In some embodiments, a spacer sequence precedes a repeat sequence in a 5′ to 3′ direction. In some embodiments, the spacer sequence(s) and the repeat sequence(s) of the guide nucleic acid are present within the same molecule. In some embodiments, the spacer(s) and repeat sequence(s) are linked directly to one another. In some embodiments, a linker is present between the spacer(s) and repeat sequences. Linkers may be any suitable linker. In some embodiments, the spacer sequence(s) and the repeat sequence(s) of the guide nucleic acid are present in separate molecules, which are joined to one another by base pairing interactions.

### Repeat Sequence

Guide nucleic acids described herein may comprise one or more repeat sequences. In some embodiments, a repeat sequence comprises a nucleotide sequence that is not complementary to a target sequence of a target nucleic acid. In some embodiments, a repeat sequence comprises a nucleotide sequence that may interact with an effector protein. In some embodiments, the repeat region may also be referred to as a “protein-binding segment.” Typically, the repeat region is adjacent to the spacer region. For example, a guide RNA that interacts with an effector protein comprises a repeat region that is 5′ of the spacer region. In some embodiments, a repeat sequence is connected to another sequence of a guide nucleic acid, such as an intermediary sequence, that is capable of non-covalently interacting with an effector protein. In some embodiments, a repeat sequence includes a nucleotide sequence that is capable of forming a guide nucleic acid-effector protein complex (e.g., a RNP complex).

In some embodiments, the repeat sequence is between 10 and 50, 12 and 48, 14 and 46, 16 and 44, and 18 and 42 nucleotides in length.

In some embodiments, a repeat sequence is adjacent to a spacer sequence. In some embodiments, a repeat sequence is followed by a spacer sequence in the 5′ to 3′ direction. In some embodiments, a repeat sequence is preceded by a spacer sequence in the 5′ to 3′ direction. In some embodiments, a repeat sequence is adjacent to an intermediary sequence. In some embodiments, a repeat sequence is 3′ to an intermediary sequence. In some embodiments, an intermediary sequence is followed by a repeat sequence, which is followed by a spacer sequence in the 5′ to 3′ direction. In some embodiments, a repeat sequence is linked to a spacer sequence and/or an intermediary sequence. In some embodiments, a guide nucleic acid comprises a repeat sequence linked to a spacer sequence and/or to an intermediary sequence, which may be a direct link or by any suitable linker, examples of which are described herein.

In some embodiments, guide nucleic acids comprise more than one repeat sequence (e.g., two or more, three or more, or four or more repeat sequences). In some embodiments, a guide nucleic acid comprises more than one repeat sequence separated by another sequence of the guide nucleic acid. For example, in some embodiments, a guide nucleic acid comprises two repeat sequences, wherein the first repeat sequence is followed by a spacer sequence, and the spacer sequence is followed by a second repeat sequence in the 5′ to 3′ direction. In some embodiments, the more than one repeat sequences are identical. In some embodiments, the more than one repeat sequences are not identical.

In some embodiments, the repeat sequence comprises two sequences that are complementary to each other and hybridize to form a double stranded RNA duplex (dsRNA duplex). In some embodiments, the two sequences are not directly linked and hybridize to form a stem loop structure. In some embodiments, the dsRNA duplex comprises 5, 10, 15, 20 or 25 base pairs (bp). In some embodiments, not all nucleotides of the dsRNA duplex are paired, and therefore the duplex forming sequence may include a bulge. In some embodiments, the repeat sequence comprises a hairpin or stem-loop structure, optionally at the 5′ portion of the repeat sequence. In some embodiments, a strand of the stem portion comprises a sequence and the other strand of the stem portion comprises a sequence that is, at least partially, complementary. In some embodiments, such sequences may have 65% to 100% complementarity (e.g., 65%, 70%, 75%, 80%, 85%, 90%, 95%, or 100% complementarity). In some embodiments, a guide nucleic acid comprises nucleotide sequence that when involved in hybridization events may hybridize over one or more segments such that intervening or adjacent segments are not involved in the hybridization event (e.g., a bulge, a loop structure or hairpin structure, etc.).

TABLE 5 provides illustrative repeat sequences for use with the compositions and methods of the disclosure. In some embodiments, the repeat sequence comprises at least 70%, at least 80%, at least 90%, at least 92%, at least 95%, at least 97%, or at least 99%, or 100% sequence identity to any one of the sequences as set forth in TABLE 5, or a reverse complement thereof. In some embodiments, the spacer sequence comprises one or more nucleobase alterations at one or more positions in any one of the sequences of TABLE 5. Alternative nucleobases can be any one or more of A, C, G. T or U, or a deletion, or an insertion.

In some instances, compositions, systems and methods described herein comprise a sequence with at least 65%, at least 70%, at least 80%, at least 90%, at least 92%, at least 95%, at least 97%, or at least 99%, or 100% sequence identity to any one of the sequences as set forth in TABLE 5. In some instances, compositions, systems and methods described herein comprise a guide nucleic acid comprising a sequence with at least 65%, at least 70%, at least 80%, at least 90%, at least 92%, at least 95%, at least 97%, or at least 99%, or 100% sequence identity to any one of the sequences as set forth in TABLE 5. In some instances, compositions, systems and methods described herein comprise a single guide nucleic acid comprising a sequence with at least 65%, at least 70%, at least 80%, at least 90%, at least 92%, at least 95%, at least 97%, or at least 99%, or 100% sequence identity to any one of the sequences as set forth in TABLE 5.

### Linker for Nucleic Acids

In some embodiments, a guide nucleic acid for use with compositions, systems, and methods described herein comprises one or more linkers, or a nucleic acid encoding one or more linkers. In some embodiments, the guide nucleic acid comprises at least one, at least two, at least three, at least four, at least five, at least six, at least seven, at least eight, at least nine, or at least ten linkers. In some embodiments, the guide nucleic acid comprises one, two, three, four, five, six, seven, eight, nine, or ten linkers. In some embodiments, the guide nucleic acid comprises more than one linker. In some embodiments, at least two of the more than one linker are the same. In some embodiments, at least two of the more than one linker are not same.

In some embodiments, a linker comprises one to ten, one to seven, one to five, one to three, two to ten, two to eight, two to six, two to four, three to ten, three to seven, three to five, four to ten, four to eight, four to six, five to ten, five to seven, six to ten, six to eight, seven to ten, or eight to ten linked nucleotides. In some embodiments, the linker comprises one, two, three, four, five, six, seven, eight, nine, or ten linked nucleotides. In some embodiments, a linker comprises a nucleotide sequence of 5′-GAAA-3′.

In some embodiments, a guide nucleic acid comprises one or more linkers connecting one or more repeat sequences. In some embodiments, the guide nucleic acid comprises one or more linkers connecting one or more repeat sequences and one or more spacer sequences. In some embodiments, the guide nucleic acid comprises at least two repeat sequences connected by a linker.

### Intermediary Sequence

Guide nucleic acids described herein may comprise one or more intermediary sequences. In general, an intermediary sequence used in the present disclosure is not transactivated or transactivating. In some embodiments, when describing an intermediary sequence in a context of a single nucleic acid system, reference is made to a nucleotide sequence in a handle sequence, wherein the nucleotide sequence is capable of, at least partially, being non-covalently bound to an effector protein to form a complex (e.g., an RNP complex). An intermediary sequence is not a transactivating nucleic acid in systems, methods, and compositions described herein.

An intermediary sequence may also be referred to as an intermediary RNA, although it may comprise deoxyribonucleotides instead of or in addition to ribonucleotides, and/or modified bases. In general, the intermediary sequence non-covalently binds to an effector protein. In some embodiments, the intermediary sequence forms a secondary structure, for example in a cell, and an effector protein binds the secondary structure.

In some embodiments, a length of the intermediary RNA sequence is at least 30, 50, 70, 90, 110, 130, 150, 170, 190, or 210 linked nucleotides. In some embodiments, a length of the intermediary RNA sequence is not greater than 30, 50, 70, 90, 110, 130, 150, 170, 190, or 210 linked nucleotides. In some embodiments, the length of the intermediary RNA sequence is about 30 to about 210, about 60 to about 210, about 90 to about 210, about 120 to about 210, about 150 to about 210, about 180 to about 210, about 30 to about 180, about 60 to about 180, about 90 to about 180, about 120 to about 180, or about 150 to about 180 linked nucleotides.

An intermediary sequence may also comprise or form a secondary structure (e.g., one or more hairpin loops) that facilitates the binding of an effector protein to a guide nucleic acid and/or modification activity of an effector protein on a target nucleic acid (e.g., a hairpin region). An intermediary sequence may comprise from 5′ to 3′, a 5′ region, a hairpin region, and a 3′ region. In some embodiments, the 5′ region may hybridize to the 3′ region. In some embodiments, the 5′ region of the intermediary sequence does not hybridize to the 3′ region.

In some embodiments, the hairpin region may comprise a first sequence, a second sequence that is reverse complementary to the first sequence, and a stem-loop linking the first sequence and the second sequence. In some embodiments, an intermediary sequence comprises a stem-loop structure comprising a stem region and a loop region. In some embodiments, the stem region is 4 to 8 linked nucleotides in length. In some embodiments, the stem region is 5 to 6 linked nucleotides in length. In some embodiments, the stem region is 4 to 5 linked nucleotides in length. In some embodiments, an intermediary sequence comprises a pseudoknot (e.g., a secondary structure comprising a stem at least partially hybridized to a second stem or half-stem secondary structure). An effector protein may interact with an intermediary sequence comprising a single stem region or multiple stem regions. In some embodiments, the nucleotide sequences of the multiple stem regions are identical to one another. In some embodiments, the nucleotide sequences of at least one of the multiple stem regions is not identical to those of the others. In some embodiments, an intermediary sequence comprises 1, 2, 3, 4, 5 or more stem regions.

In some embodiments, an intermediary sequence comprises a nucleotide sequence that is at least 65%, at least 70%, at least 75%, at least 80%, at least 85%, at least 90%, at least 95%, at least 97%, or at least 98%, at least 99%, or 100% identical to any one of the intermediary sequences in TABLE 5.1. In some embodiments, an intermediary sequence comprises at least 5, at least 6, at least 7, at least 8, at least 9, at least 10, at least 20, at least 30, at least 40, at least 50, at least 60, at least 70, at least 80, at least 90, at least 100, at least 110, at least 120, at least 130, or at least 140 contiguous nucleotides of any one of the intermediary sequences recited in TABLE 5.1.

### Handle Sequence

Guide nucleic acids described herein may comprise one or more handle sequences. In some embodiments, when describing a handle sequence reference is made to a sequence of nucleotides in a single guide RNA (sgRNA), that is: 1) capable of being non-covalently bound by an effector protein and 2) connects the portion of the sgRNA capable of being non-covalently bound by an effector protein to a nucleotide sequence that is hybridizable to a target nucleic acid.

In general, the handle sequence comprises an intermediary sequence, that is capable of being non-covalently bound by an effector protein. In some instances, the handle sequence further comprises a repeat sequence. In such instances, the intermediary sequence or a combination of the intermediary RNA and the repeat sequence is capable of being non-covalently bound by an effector protein. In some embodiments, the intermediary sequence is at the 3′-end of the handle sequence. In some embodiments, the intermediary sequence is at the 5′-end of the handle sequence. Additionally, or alternatively, in some embodiments, the handle sequence further comprises one or more of linkers and repeat sequences. In such instances, at least a portion of an intermediary sequence, or both of at least a portion of the intermediary sequence and at least a portion of repeat sequence, non-covalently interacts with an effector protein. In some embodiments, an intermediary sequence and repeat sequence are directly linked (e.g., covalently linked, such as through a phosphodiester bond). In some embodiments, the intermediary sequence and repeat sequence are linked by a suitable linker, examples of which are provided herein. In some embodiments, the linker comprises a sequence of 5′-GAAA-3′. In some embodiments, the intermediary sequence is 5′ to the repeat sequence. In some embodiments, the intermediary sequence is 5′ to the linker. In some embodiments, the intermediary sequence is 3′ to the repeat sequence. In some embodiments, the intermediary sequence is 3′ to the linker. In some embodiments, the repeat sequence is 3′ to the linker. In some embodiments, the repeat sequence is 5′ to the linker. In general, a single guide nucleic acid, also referred to as a single guide RNA (sgRNA), comprises a handle sequence comprising an intermediary sequence, and optionally one or more of a repeat sequence and a linker.

A handle sequence may comprise or form a secondary structure (e.g., one or more hairpin loops) that facilitates the binding of an effector protein to a guide nucleic acid and/or modification activity of an effector protein on a target nucleic acid (e.g., a hairpin region). In some embodiments, handle sequences comprise a stem-loop structure comprising a stem region and a loop region. In some embodiments, the stem region is 4 to 8 linked nucleotides in length. In some embodiments, the stem region is 5 to 6 linked nucleotides in length. In some embodiments, the stem region is 4 to 5 linked nucleotides in length. In some embodiments, the handle sequence comprises a pseudoknot (e.g., a secondary structure comprising a stem at least partially hybridized to a second stem or half-stem secondary structure). An effector protein may recognize a handle sequence comprising multiple stem regions. In some embodiments, the nucleotide sequences of the multiple stem regions are identical to one another. In some embodiments, the nucleotide sequences of at least one of the multiple stem regions is not identical to those of the others. In some embodiments, the handle sequence comprises at least 2, at least 3, at least 4, or at least 5 stem regions.

In some embodiments, a length of the handle sequence is at least 30, 50, 70, 90, 110, 130, 150, 170, 190, or 210 linked nucleotides. In some embodiments, a length of the handle sequence is not greater than 30, 50, 70, 90, 110, 130, 150, 170, 190, or 210 linked nucleotides. In some embodiments, the length of the handle sequence is about 30 to about 210, about 60 to about 210, about 90 to about 210, about 120 to about 210, about 150 to about 210, about 180 to about 210, about 30 to about 180, about 60 to about 180, about 90 to about 180, about 120 to about 180, or about 150 to about 180 linked nucleotides.

In some embodiments, a handle sequence comprises a nucleotide sequence that is at least 65%, at least 70%, at least 75%, at least 80%, at least 85%, at least 90%, at least 95%, at least 97%, or at least 98%, at least 99%, or 100% identical to any one of the handle sequences in TABLE 6. In some embodiments, a handle sequence comprises at least 5, at least 6, at least 7, at least 8, at least 9, at least 10, at least 20, at least 30, at least 40, at least 50, at least 60, at least 70, at least 80, at least 90, at least 100, at least 110, at least 120, at least 130, or at least 140 contiguous nucleotides of any one of the handle sequences recited in TABLE 6.

### A Single Nucleic Acid System

In some embodiments, compositions, systems and methods described herein comprise a single nucleic acid system comprising a guide nucleic acid or a nucleotide sequence encoding the guide nucleic acid, and one or more effector proteins or a nucleotide sequence encoding the one or more effector proteins. In some embodiments, when describing a single nucleic acid system, reference is made to a system that uses a guide nucleic acid complexed with one or more polypeptides described herein, wherein the complex is capable of interacting with a target nucleic acid in a sequence specific manner, and wherein the guide nucleic acid is capable of non-covalently interacting with the one or more polypeptides described herein, and wherein the guide nucleic acid is capable of hybridizing with a target sequence of the target nucleic acid. A single nucleic acid system lacks a duplex of a guide nucleic acid as hybridized to a second nucleic acid, wherein in such a duplex the second nucleic acid, and not the guide nucleic acid, is capable of interacting with the effector protein

In some embodiments, a second region of the guide nucleic acid non-covalently interacts with the one or more polypeptides described herein. In some embodiments, a first region of the guide nucleic acid hybridizes with a target sequence of the target nucleic acid. In the single nucleic acid system having a complex of the guide nucleic acid and the effector protein, the effector protein is not transactivated by the guide nucleic acid. In other words, activity of effector protein does not require binding to a second non-target nucleic acid molecule. An exemplary guide nucleic acid for a single nucleic acid system is a crRNA or a sgRNA.

crRNA

In some embodiments, a guide nucleic acid comprises a crRNA. In some embodiments, the guide nucleic acid is the crRNA. In some embodiments, a crRNA comprises a first region and a second region, wherein the second region of the crRNA comprises a repeat sequence, and the first region of the crRNA comprises a spacer sequence. In general, a crRNA comprises a spacer region comprising the spacer sequence that hybridizes to a target sequence of a target nucleic acid, and a repeat region comprising the repeat sequence that interacts with the effector protein. In some embodiments, the repeat sequence and the spacer sequences are directly connected to each other (e.g., covalent bond (phosphodiester bond)). In some embodiments, the repeat sequence and the spacer sequence are connected by a linker. In some embodiments, the guide RNA does not comprise a tracrRNA. In such embodiments, the guide nucleic acid is not transactivated or transactivating. In some embodiments, the crRNA of the guide nucleic acid comprises a repeat region and a spacer region, wherein the repeat region binds to the effector protein and the spacer region hybridizes to a target sequence of the target nucleic acid. The repeat sequence of the crRNA may interact with an effector protein, allowing for the guide nucleic acid and the effector protein to form an RNP complex.

A crRNA may be the product of processing of a longer precursor CRISPR RNA (pre-crRNA) transcribed from the CRISPR array by cleavage of the pre-crRNA within each direct repeat sequence to afford shorter, mature crRNAs. A crRNA may be generated by a variety of mechanisms, including the use of dedicated endonucleases (e.g., Cas6 or Cas5d in Type I and III systems), coupling of a host endonuclease (e.g., RNase III) with tracrRNA (Type II systems), or a ribonuclease activity endogenous to the effector protein itself (e.g., Cpf1, from Type V systems). A crRNA may also be specifically generated outside of processing of a pre-crRNA and individually contacted to an effector protein in vivo or in vitro.

In some embodiments, a crRNA is useful as a single nucleic acid system for compositions, methods, and systems described herein or as part of a single nucleic acid system for compositions, methods, and systems described herein. In some embodiments, a crRNA is useful as part of a single nucleic acid system for compositions, methods, and systems described herein. In such embodiments, a single nucleic acid system comprises a guide nucleic acid comprising a crRNA wherein, a repeat sequence of a crRNA is capable of connecting a crRNA to an effector protein. In some embodiments, a single nucleic acid system comprises a guide nucleic acid comprising a crRNA linked to another nucleotide sequence that is capable of being non-covalently bond by an effector protein. In such embodiments, a repeat sequence of a crRNA can be linked to an intermediary RNA. In some embodiments, a single nucleic acid system comprises a guide nucleic acid comprising a crRNA and an intermediary RNA.

A crRNA may include deoxyribonucleosides, ribonucleosides, chemically modified nucleosides, or any combination thereof. In some embodiments, a crRNA comprises about: 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 linked nucleotides. In some embodiments, a crRNA comprises at least: 10, 15, 20, 25, 30, 35, 40, 45, 50, 55, 60 linked nucleotides. In some embodiments, the length of the crRNA is about 20 to about 120 linked nucleotides. In some embodiments, the length of a crRNA is about 20 to about 100, about 30 to about 100, about 40 to about 100, about 40 to about 90, about 40 to about 80, about 40 to about 70, about 40 to about 60, about 40 to about 50, about 50 to about 90, about 50 to about 80, about 50 to about 70, or about 50 to about 60 linked nucleotides. In some embodiments, the length of a crRNA is about 20, about 25, about 30, about 35, about 40, about 45, about 50, about 55, about 60, about 65, about 70 or about 75 linked nucleotides.

In some instances, compositions disclosed herein comprises a crRNA comprising a spacer sequence that is at least 70%, at least 75%, at least 80%, at least 85%, at least 90%, at least 95%, or 100% identical to any one of the sequences as set forth in TABLE 4 and comprising a repeat sequence that is at least 70%, at least 75%, at least 80%, at least 85%, at least 90%, at least 95%, or 100% identical to any one of the sequences of TABLE 5.

In some instances, compositions disclosed herein comprises an effector protein comprising an amino acid sequence that is at least 70%, at least 75%, at least 80%, at least 85%, at least 90%, at least 95%, at least 99%, or 100% identical to any one of the sequences as set forth in TABLE 1; a crRNA comprising a spacer sequence that is at least 70%, at least 75%, at least 80%, at least 85%, at least 90%, at least 95%, or 100% identical to any one of the sequences as set forth in TABLE 4 and comprising a repeat sequence that is at least 70%, at least 75%, at least 80%, at least 85%, at least 90%, at least 95%, or 100% identical to any one of the sequences of TABLE 5.

TABLE 7 also provides exemplary compositions comprising an effector protein described herein and crRNAs. Each row in TABLE 7 represents an exemplary composition comprising an effector protein corresponding to an amino acid sequence as set forth in TABLE 1, recognizing a PAM sequence as set forth in TABLE 7 and a guide nucleic acid, wherein the guide nucleic acid is a crRNA. In some instances, the gRNA comprises a nucleotide sequence of any one of the crRNA sequences of TABLE 7. In some instances, the nucleotide sequence of the guide nucleic acid is at least 65%, at least 70%, at least 75%, at least 80%, at least 85%, at least 90%, at least 95%, at least 97%, or at least 98%, at least 99%, or 100% identical to any one of the crRNA sequences of TABLE 7.

In some embodiments, a crRNA comprises a nucleotide sequence that is at least 65%, at least 70%, at least 75%, at least 80%, at least 85%, at least 90%, at least 95%, at least 97%, or at least 98%, at least 99%, or 100% identical to any one of the crRNA sequences in TABLE 7. In some embodiments, a crRNA sequence comprises a repeat sequence that is at least 65%, at least 70%, at least 75%, at least 80%, at least 85%, at least 90%, at least 95%, at least 97%, or at least 98%, at least 99%, or 100% identical to any one of the sequences set forth in TABLE 5, and a spacer sequence that is at least 65%, at least 70%, at least 75%, at least 80%, at least 85%, at least 90%, at least 95%, at least 97%, or at least 98%, at least 99%, or 100% identical to any one of the sequences set forth in TABLE 4. In some embodiments, a crRNA comprises at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 contiguous nucleotides of any one of the crRNA sequences recited in TABLE 7. In some embodiments, a crRNA sequence comprises at least 5, at least 6, at least 7, at least 8, at least 9, or at least 10 contiguous nucleotides of any one of the repeat sequences recited in TABLE 5, and at least 5, at least 6, at least 7, at least 8, at least 9, or at least 10 contiguous nucleotides of any one of the spacer sequences recited in TABLE 4.

sgRNA

In some embodiments, a guide nucleic acid comprises a sgRNA. In some embodiments, when describing a single guide nucleic acid, a single guide RNA, or an sgRNA, in the context of a single nucleic acid system, reference is made to a guide nucleic acid, wherein the guide nucleic acid is a single polynucleotide chain having all the required sequence for a functional complex with an effector protein (e.g., being bound by an effector protein, including in some instances activating the effector protein, and hybridizing to a target nucleic acid, without the need for a second nucleic acid molecule). For example, an sgRNA can have two or more linked guide nucleic acid components (e.g., an intermediary sequence, a repeat sequence, a spacer sequence and optionally a linker, or a handle sequence and a spacer sequence)

In some embodiments, a guide nucleic acid is a sgRNA. In some embodiments, a sgRNA comprises a first region and a second region, wherein the second region comprises a handle sequence and the first region comprises a spacer sequence. In some embodiments, the handle sequence and the spacer sequences are directly connected to each other (e.g., covalent bond (phosphodiester bond)). In some embodiments, the handle sequence and the spacer sequence are connected by a linker.

In some embodiments, a sgRNA comprises one or more of one or more of a handle sequence, an intermediary sequence, a crRNA, a repeat sequence, a spacer sequence, a linker, or combinations thereof. For example, a sgRNA comprises a handle sequence and a spacer sequence: an intermediary sequence and an crRNA; an intermediary sequence, a repeat sequence and a spacer sequence; and the like.

In some embodiments, a sgRNA comprises an intermediary sequence and an crRNA. In some embodiments, an intermediary sequence is 5′ to a crRNA in an sgRNA. In some embodiments, a sgRNA comprises a linked intermediary sequence and crRNA. In some embodiments, an intermediary sequence and a crRNA are linked in an sgRNA directly (e.g., covalently linked, such as through a phosphodiester bond) In some embodiments, an intermediary sequence and a crRNA are linked in an sgRNA by any suitable linker, examples of which are provided herein.

In some embodiments, a sgRNA comprises a handle sequence and a spacer sequence. In some embodiments, a handle sequence is 5′ to a spacer sequence in an sgRNA. In some embodiments, a sgRNA comprises a linked handle sequence and spacer sequence. In some embodiments, a handle sequence and a spacer sequence are linked in an sgRNA directly (e.g., covalently linked, such as through a phosphodiester bond) In some embodiments, a handle sequence and a spacer sequence are linked in an sgRNA by any suitable linker, examples of which are provided herein.

In some embodiments, a sgRNA comprises an intermediary sequence, a repeat sequence, and a spacer sequence. In some embodiments, an intermediary sequence is 5′ to a repeat sequence in an sgRNA. In some embodiments, a sgRNA comprises a linked intermediary sequence and repeat sequence. In some embodiments, an intermediary sequence and a repeat sequence are linked in an sgRNA directly (e.g., covalently linked, such as through a phosphodiester bond) In some embodiments, an intermediary sequence and a repeat sequence are linked in an sgRNA by any suitable linker, examples of which are provided herein. In some embodiments, a repeat sequence is 5′ to a spacer sequence in an sgRNA. In some embodiments, a sgRNA comprises a linked repeat sequence and spacer sequence. In some embodiments, a repeat sequence and a spacer sequence are linked in an sgRNA directly (e.g, covalently linked, such as through a phosphodiester bond) In some embodiments, a repeat sequence and a spacer sequence are linked in an sgRNA by any suitable linker, examples of which are provided herein.

In some embodiments, a sgRNA sequence comprises a nucleotide sequence that is at least 65%, at least 70%, at least 75%, at least 80%, at least 85%, at least 90%, at least 95%, at least 97%, or at least 98%, at least 99%, or 100% identical to any one of the sequences in TABLE 4. TABLE 5, TABLE 5.1, TABLE 6, TABLE 7, and TABLE 8, or a combination thereof. In some embodiments, a sgRNA sequence comprises a nucleotide sequence that is at least 65%, at least 70%, at least 75%, at least 80%, at least 85%, at least 90%, at least 95%, at least 97%, or at least 98%, at least 99%, or 100% identical to any one of the sequences in TABLE 8. In some embodiments, a sgRNA sequence comprises a handle sequence that is at least 65%, at least 70%, at least 75%, at least 80%, at least 85%, at least 90%, at least 95%, at least 97%, or at least 98%, at least 99%, or 100% identical to any one of the sequences in TABLE 6, and a spacer sequence that is at least 65%, at least 70%, at least 75%, at least 80%, at least 85%, at least 90%, at least 95%, at least 97%, or at least 98%, at least 99%, or 100% identical to any one of the sequences in TABLE 5. In some embodiments, a sgRNA comprises at least 5, at least 6, at least 7, at least 8, at least 9, or at least 10 contiguous nucleotides of any one of the sgRNA sequences recited in TABLE 8. In some embodiments, a sgRNA sequence comprises a handle sequence comprising at least 5, at least 6, at least 7, at least 8, at least 9, or at least 10 contiguous nucleotides of any one of the sequences set forth in TABLE 6, and a spacer sequence comprising at least 5, at least 6, at least 7, at least 8, at least 9, or at least 10 contiguous nucleotides of any one of the sequences set forth in TABLE 5.

### A Dual Nucleic Acid System

In some embodiments, compositions, systems and methods described herein comprise a dual nucleic acid system comprising a crRNA or a nucleotide sequence encoding the crRNA, a tracrRNA or a nucleotide sequence encoding the tracrRNA, and one or more effector protein or a nucleotide sequence encoding the one or more effector protein, wherein the crRNA and the tracrRNA are separate, unlinked molecules, wherein a repeat hybridization region of the tracrRNA is capable of hybridizing with an equal length portion of the crRNA to form a tracrRNA-crRNA duplex, wherein the equal length portion of the crRNA does not include a spacer sequence of the crRNA, and wherein the spacer sequence is capable of hybridizing to a target sequence of the target nucleic acid. In the dual nucleic acid system having a complex of the guide nucleic acid, tracrRNA, and the effector protein, the effector protein is transactivated by the tracrRNA. In some embodiments, when describing transactivation (and grammatical equivalents thereof) in the context of a dual nucleic acid system refers to an outcome of the system, wherein a polypeptide is enabled to have a binding and/or nuclease activity on a target nucleic acid, by a tracrRNA or a tracrRNA-crRNA duplex. In other words, activity of effector protein requires binding to a tracrRNA molecule. In some embodiments, the dual nucleic acid system comprises a guide nucleic acid and a tracrRNA, wherein the tracrRNA is an additional nucleic acid capable of at least partially hybridizing to the first region of the guide nucleic acid. In some embodiments, the tracrRNA or additional nucleic acid is capable of at least partially hybridizing to the 5′ end of the second region of the guide nucleic acid.

In some embodiments, a repeat hybridization sequence is at the 3′ end of a tracrRNA. In some embodiments, a repeat hybridization sequence may have a length of about 1, about 2, about 3, about 4, about 5, about 6, about 7, about 8, about 9, about 10, about 12, about 14, about 16, about 18, or about 20 linked nucleotides. In some embodiments, the length of the repeat hybridization sequence is 1 to 20 linked nucleotides.

A tracrRNA and/or tracrRNA-crRNA duplex may form a secondary structure that facilitates the binding of an effector protein to a tracrRNA or a tracrRNA-crRNA. In some embodiments, the secondary structure modifies activity of the effector protein on a target nucleic acid. In some embodiments, the secondary structure comprises a stem-loop structure comprising a stem region and a loop region. In some embodiments, the stem region is 4 to 8 linked nucleotides in length. In some embodiments, the stem region is 5 to 6 linked nucleotides in length. In some embodiments, the stem region is 4 to 5 linked nucleotides in length. In some embodiments, the secondary structure comprises a pseudoknot (e.g., a secondary structure comprising a stem at least partially hybridized to a second stem or half-stem secondary structure). An effector protein may recognize a secondary structure comprising multiple stem regions. In some embodiments, nucleotide sequences of the multiple stem regions are identical to one another. In some embodiments, the nucleotide sequences of at least one of the multiple stem regions is not identical to those of the others. In some embodiments, the secondary structure comprises at least two, at least three, at least four, or at least five stem regions. In some embodiments, the secondary structure comprises one or more loops. In some embodiments, the secondary structure comprises at least one, at least two, at least three, at least four, or at least five loops.

### III. Engineered Modifications

Polypeptides (e.g., effector proteins) and nucleic acids (e.g., engineered guide nucleic acids) can be further modified as described herein. In some embodiments, when describing an engineered modification reference is made to a structural change of one or more nucleic acid residues of a nucleotide sequence or one or more amino acid residue of an amino acid sequence, such as chemical modification of one or more nucleobases; or a chemical change to the phosphate backbone, a nucleotide, a nucleobase, or a nucleoside. Such modifications can be made to an effector protein amino acid sequence or guide nucleic acid nucleotide sequence, or any sequence disclosed herein (e.g., a nucleic acid encoding an effector protein or a nucleic acid that encodes a guide nucleic acid). Methods of modifying a nucleic acid or amino acid sequence are known. One of ordinary skill in the art will appreciate that the engineered modification(s) may be located at any position(s) of a nucleic acid such that the function of the nucleic acid, protein, composition or system is not substantially decreased. Nucleic acids provided herein can be prepared according to any available technique including, but not limited to chemical synthesis, enzymatic synthesis, which is generally termed in vitro-transcription, cloning, enzymatic, or chemical cleavage, etc. In some instances, the nucleic acids provided herein are not uniformly modified along the entire length of the molecule. Different nucleotide modifications and/or backbone structures can exist at various positions within the nucleic acid

Examples are modifications that do not alter the primary sequence of the polypeptides or nucleic acids, such as chemical derivatization of polypeptides (e.g., acylation, acetylation, carboxylation, amidation, etc.), or modifications that do alter the primary sequence of the polypeptide or nucleic acid. Also included are polypeptides that have a modified glycosylation pattern (e.g., those made by: modifying the glycosylation patterns of a polypeptide during its synthesis and processing or in further processing steps; by exposing the polypeptide to enzymes which affect glycosylation, such as mammalian glycosylating or deglycosylating enzymes). Also embraced are polypeptides that have phosphorylated amino acid residues (e.g., phosphotyrosine, phosphoserine, or phosphothreonine).

Modifications disclosed herein can also include modification of described polypeptides and/or guide nucleic acids through any suitable method, such as molecular biological techniques and/or synthetic chemistry, to improve their resistance to proteolytic degradation, to change the target sequence specificity, to optimize solubility properties, to alter protein activity (e.g., transcription modulatory activity, enzymatic activity, etc.) or to render them more suitable for their intended purpose (e.g., in vivo administration, in vitro methods, or ex vivo applications). Analogs of such polypeptides include those containing residues other than naturally occurring L-amino acids, e.g. D-amino acids or non-naturally occurring synthetic amino acids. D-amino acids may be substituted for some or all of the amino acid residues. Modifications can also include modifications with non-naturally occurring unnatural amino acids. The particular sequence and the manner of preparation will be determined by convenience, economics, purity required, and the like.

Modifications can further include the introduction of various groups to polypeptides and/or guide nucleic acids described herein. For example, groups can be introduced during synthesis or during expression of a polypeptide (e.g., an effector protein), which allow for linking to other molecules or to a surface. Thus, e.g., cysteines may be used to make thioethers, histidines for linking to a metal ion complex, carboxyl groups for forming amides or esters, amino groups for forming amides, and the like.

Modifications can further include changing of nucleic acids described herein (e.g., engineered guide nucleic acids) to provide the nucleic acid with a new or enhanced feature, such as improved stability. Such modifications of a nucleic acid include a base editing, a base modification, a backbone modification, a sugar modification, or combinations thereof. In some embodiments, the modifications can be of one or more nucleotides, nucleosides, or nucleobases in a nucleic acid.

In some embodiments, nucleic acids (e.g., nucleic acids encoding effector proteins, engineered guide nucleic acids, or nucleic acids encoding engineered guide nucleic acids) described herein comprise one or more modifications comprising: 2′O-methyl modified nucleotides (e.g., 2′-O-Methyl (2′OMe) sugar modifications); 2′ fluoro modified nucleotides (e.g., 2′-fluoro (2′-F) sugar modifications); locked nucleic acid (LNA) modified nucleotides; peptide nucleic acid (PNA) modified nucleotides; nucleotides with phosphorothioate linkages; a 5′ cap (e.g., a 7-methylguanylate cap (m7G)), phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkyl phosphoramidates, phosphorodiamidates, thionophosphor amidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′. 5′ to 5′ or 2′ to 2′ linkage; phosphorothioate and/or heteroatom internucleoside linkages, such as —CH₂—NH—O—CH₂—, —CH₂—N(CH₃)—O—CH₂— (known as a methylene (methylimino) or MMI backbone), —CH₂—O—N(CH₃)—CH₂—, —CH₂—N(CH₃)—N(CH₃)—CH₂— and —O—N(CH₃)—CH—CH₂-(wherein the native phosphodiester internucleotide linkage is represented as —O—P(═O) (OH)—O—CH₂—); morpholino linkages (formed in part from the sugar portion of a nucleoside); morpholino backbones; phosphorodiamidate or other non-phosphodiester internucleoside linkages; siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; other backbone modifications having mixed N, O, S and CH₂ component parts; and combinations thereof.

### IV. Vectors and Multiplexed Expression Vectors

Compositions, systems, and methods described herein comprise a vector or a use thereof. A vector can comprise a nucleic acid of interest. In some embodiments, the nucleic acid of interest comprises one or more components of a composition or system described herein. In some embodiments, the nucleic acid of interest comprises a nucleotide sequence that encodes one or more components of the composition or system described herein. In some embodiments, one or more components comprises a polypeptide(s), guide nucleic acid(s), target nucleic acid(s), and donor nucleic acid(s). In some embodiments, the component comprises a nucleic acid encoding an effector protein, a donor nucleic acid, and a guide nucleic acid or a nucleic acid encoding the guide nucleic acid. In some embodiments, a vector may be part of a vector system. The vector system may comprise a library of vectors each encoding one or more component of a composition or system described herein. In some embodiments, two or more component described herein (e.g., an effector protein and a guide nucleic acid, a donor nucleic acid, and/or a target nucleic acid) are encoded or provided by the same vector. In some embodiments, components described herein (e.g., an effector protein, a guide nucleic acid, a donor nucleic acid, and/or a target nucleic acid) are encoded or provided by the same vector. In some embodiments, components described herein (e.g., an effector protein, a guide nucleic acid, a donor nucleic acid, and/or a target nucleic acid) are each encoded or provided by different vectors of the system.

In some embodiments, a vector comprises a nucleotide sequence encoding one or more effector proteins as described herein. In some embodiments, the one or more effector proteins comprise at least two effector proteins. In some embodiments, the at least two effector protein are the same. In some embodiments, the at least two effector proteins are different from each other. In some embodiments, the nucleotide sequence is operably linked to a promoter that is operable in a target cell, such as a eukaryotic cell. In some embodiments, the vector comprises the nucleotide sequence encoding 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more effector proteins.

In some embodiments, a vector may encode one or more of any system components, including but not limited to effector proteins, guide nucleic acids, donor nucleic acids, and target nucleic acids as described herein. In some embodiments, a system component encoding sequence is operably linked to a promoter that is operable in a target cell, such as a eukaryotic cell. In some embodiments, a vector may encode 1, 2, 3, 4 or more of any system components. For example, a vector may encode two or more guide nucleic acids, wherein each guide nucleic acid comprises a different sequence. A vector may encode an effector protein and a guide nucleic acid. A vector may encode an effector protein, a guide nucleic acid, and a donor nucleic acid.

In some embodiments, a vector comprises one or more guide nucleic acids, or a nucleotide sequence encoding the one or more guide nucleic acids as described herein. In some embodiments, the one or more guide nucleic acids comprise at least two guide nucleic acids. In some embodiments, the at least two guide nucleic acids are the same. In some embodiments, the at least two guide nucleic acids are different from each other. In some embodiments, the guide nucleic acid or the nucleotide sequence encoding the guide nucleic acid is operably linked to a promoter that is operable in a target cell, such as a eukaryotic cell. In some embodiments, the vector comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more guide nucleic acids. In some embodiments, the vector comprises a nucleotide sequence encoding 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more guide nucleic acids.

In some embodiments, a vector comprises one or more donor nucleic acids as described herein. In some embodiments, the one or more donor nucleic acids comprise at least two donor nucleic acids. In some embodiments, the at least two donor nucleic acids are the same. In some embodiments, the at least two donor nucleic acids are different from each other. In some embodiments, the vector comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more donor nucleic acids.

In some embodiments, a vector may comprise or encode one or more regulatory elements. Regulatory elements may refer to transcriptional and translational control sequences, such as promoters, enhancers, polyadenylation signals, terminators, protein degradation signals, and the like, that provide for and/or regulate transcription of a non-coding sequence or a coding sequence and/or regulate translation of an encoded polypeptide. In some embodiments, a vector may comprise or encode for one or more additional elements, such as, for example, replication origins, antibiotic resistance (or a nucleic acid encoding the same), a tag (or a nucleic acid encoding the same), selectable markers, and the like. In some embodiments, a vector comprises or encodes for one or more elements, such as, for example, ribosome binding sites, and RNA splice sites.

Vectors described herein can encode a promoter—a regulatory region on a nucleic acid, such as a DNA sequence, capable of initiating transcription of a downstream (3′ direction) coding or non-coding sequence. A promoter can be linked at its 3′ terminus to a nucleic acid, the expression or transcription of which is desired, and extends upstream (5′ direction) to include bases or elements necessary to initiate transcription or induce expression, which could be measured at a detectable level. A promoter can comprise a nucleotide sequence, referred to herein as a “promoter sequence”. The promoter sequence can include a transcription initiation site, and one or more protein binding domains responsible for the binding of transcription machinery, such as RNA polymerase. When eukaryotic promoters are used, such promoters can contain “TATA” boxes and “CAT” boxes. Various promoters, including inducible promoters, may be used to drive expression, i.e., transcriptional activation, of the nucleic acid of interest. Accordingly, in some embodiments, the nucleic acid of interest can be operably linked to a promoter.

Promotors may be any suitable type of promoter envisioned for the compositions, systems, and methods described herein. Examples include constitutively active promoters (e.g., CMV promoter), inducible promoters (e.g., heat shock promoter, tetracycline-regulated promoter, steroid-regulated promoter, metal-regulated promoter, estrogen receptor-regulated promoter, etc.), spatially restricted and/or temporally restricted promoters (e.g., a tissue specific promoter, a cell type specific promoter, etc.), etc. Suitable promoters include, but are not limited to: SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6), an enhanced U6 promoter, and a human H1 promoter (H1). By transcriptional activation, it is intended that transcription will be increased above basal levels in the target cell by 2 fold, 5 fold, 10 fold, 50 fold, by 100 fold, 500 fold, or by 1000 fold, or more. In addition, vectors used for providing a nucleic acid that, when transcribed, produces a guide nucleic acid and/or a nucleic acid that encodes an effector protein to a cell may include nucleic acid sequences that encode for selectable markers in the target cells, so as to identify cells that have taken up the guide nucleic acid and/or the effector protein.

In general, vectors provided herein comprise at least one promotor or a combination of promoters driving expression or transcription of one or more genome editing tools described herein. In some embodiments, the vector comprises a nucleotide sequence of a promoter. In some embodiments, the vector comprises two promoters. In some embodiments, the vector comprises three promoters. In some embodiments, a length of the promoter is less than about 500, less than about 400, less than about 300, or less than about 200 linked nucleotides. In some embodiments, a length of the promoter is at least 100, at least 200, at least 300, at least 400, or at least 500 linked nucleotides. Non-limiting examples of promoters include CMV, 7SK, EF1a, RPBSA, hPGK, EFS, SV40, PGK1, Ubc, human beta actin, TRE, UAS, Ac5, Polyhedrin, CaMKIIa, GAL1-10, H1, TEF1, GDS, ADH1, CaMV35S, HSV TK, Ubi, U6, MNDU3, MSCV, MND, Ck8c, SPC5-12, Desmin and CAG.

In some embodiments, the promoter is a constitutive promoter. In some embodiments, the promoter is an inducible promoter. In some embodiments, the inducible promoter only drives expression of its corresponding coding sequence (e.g., polypeptide or guide nucleic acid) when a signal is present, e.g., a hormonc, a small molecule, a peptide. Non-limiting examples of inducible promoters are the T7 RNA polymerase promoter, the T3 RNA polymerase promoter, the Isopropyl-beta-D-thiogalactopyranoside (IPTG)-regulated promoter, a lactose induced promoter, a heat shock promoter, a tetracycline-regulated promoter (tetracycline-inducible or tetracycline-repressible), a steroid regulated promoter, a metal-regulated promoter, and an estrogen receptor-regulated promoter. In some embodiments, the promoter is an activation-inducible promoter, such as a CD69 promoter, as described further in Kulemzin et al., (2019), BMC Med Genomics, 12:44. In some embodiments, the promoter for expressing effector protein is a muscle-specific promoter. In some embodiments, the muscle-specific promoter comprises Ck8e, SPC5-12, or Desmin promoter sequence. In some embodiments, the promoter for expressing effector protein is a ubiquitous promoter. In some embodiments, the ubiquitous promoter comprises MND or CAG promoter sequence.

In some embodiments, the promoters are prokaryotic promoters (e.g., drive expression of a gene in a prokaryotic cell). In some embodiments, the promoters are eukaryotic promoters, (e.g., drive expression of a gene in a eukaryotic cell). In some embodiments, the promoter is EF1a. In some embodiments, the promoter is ubiquitin. In some embodiments, vectors are bicistronic or polycistronic vector (e.g., having or involving two or more loci responsible for generating a protein) having an internal ribosome entry site (IRES) is for translation initiation in a cap-independent manner.

In some embodiments, a vector described herein is a nucleic acid expression vector. In some embodiments, a vector described herein is a recombinant expression vector. In some embodiments, a vector described herein is a messenger RNA.

In some embodiments, a vector described herein is a delivery vector. In some embodiments, the delivery vector is a eukaryotic vector, a prokaryotic vector (e.g., a bacterial vector) a viral vector, or any combination thereof. In some embodiments, the delivery vehicle is a non-viral vector. In some embodiments, the delivery vector is a plasmid. In some embodiments, the plasmid comprises DNA. In some embodiments, the plasmid comprises RNA. In some embodiments, the plasmid comprises circular double-stranded DNA. In some embodiments, the plasmid is linear. In some embodiments, the plasmid comprises one or more coding sequences of interest and one or more regulatory elements. In some embodiments, the plasmid comprises a bacterial backbone containing an origin of replication and an antibiotic resistance gene or other selectable marker for plasmid amplification in bacteria. In some embodiments, the plasmid is a minicircle plasmid. In some embodiments, the plasmid contains one or more genes that provide a selective marker to induce a target cell to retain the plasmid. In some examples, the plasmids are engineered through synthetic or other suitable means known in the art. For example, in some embodiments, the genetic elements are assembled by restriction digest of the desired genetic sequence from a donor plasmid or organism to produce ends of the DNA which is then be readily ligated to another genetic sequence.

In some embodiments, vectors comprise an enhancer. Enhancers are nucleotide sequences that have the effect of enhancing promoter activity. In some embodiments, enhancers augment transcription regardless of the orientation of their sequence. In some embodiments, enhancers activate transcription from a distance of several kilo basepairs. Furthermore, enhancers are located optionally upstream or downstream of a gene region to be transcribed, and/or located within the gene, to activate the transcription. Exemplary enhancers include, but are not limited to, WPRE; CMV enhancers; the R—U5′ segment in LTR of HTLV-I.

### Administration of a Non-Viral Vector

In some embodiments, an administration of a non-viral vector comprises contacting a cell, such as a host cell, with the non-viral vector. In some embodiments, a physical method or a chemical method is employed for delivering the vector into the cell. Exemplary physical methods include electroporation, gene gun, sonoporation, magnetofection, or hydrodynamic delivery. Exemplary chemical methods include delivery of the recombinant polynucleotide by liposomes such as, cationic lipids or neutral lipids; lipofection; dendrimers; lipid nanoparticle (LNP); or cell-penetrating peptides.

In some embodiments, a vector is administered as part of a method of nucleic acid detection, editing, and/or treatment as described herein. In some embodiments, a vector is administered in a single vehicle, such as a single expression vector. In some embodiments, at least two of the three components, a nucleic acid encoding one or more effector proteins, one or more donor nucleic acids, and one or more guide nucleic acids or a nucleic acid encoding the one or more guide nucleic acid, are provided in the single expression vector. In some embodiments, components, such as a guide nucleic acid and an effector protein, are encoded by the same vector. In some embodiments, an effector protein (or a nucleic acid encoding same) and/or an engineered guide nucleic acid (or a nucleic acid that, when transcribed, produces same) are not co-administered with donor nucleic acid in a single vehicle. In some embodiments, an effector protein (or a nucleic acid encoding same), an engineered guide nucleic acid (or a nucleic acid that, when transcribed, produces same), and/or donor nucleic acid are administered in one or more or two or more vehicles, such as one or more, or two or more expression vectors.

In some embodiments, a vector system is administered as part of a method of nucleic acid detection, editing, and/or treatment as described herein, wherein at least two vectors are co-administered. In some embodiments, the at least two vectors comprise different components. In some embodiments, the at least two vectors comprise the same component having different sequences. In some embodiments, at least one of the three components, a nucleic acid encoding one or more effector proteins, one or more donor nucleic acids, and one or more guide nucleic acids or a nucleic acid encoding the one or more guide nucleic acids, or a variant thereof is provided in a different vector. In some embodiments, the nucleic acid encoding the effector protein, and a guide nucleic acid or a nucleic acid encoding the guide nucleic acid are provided in different vectors. In some embodiments, the donor nucleic acid is encoded by a different vector than the vector encoding the effector protein and the guide nucleic acid.

### Lipid Particles and Non-Viral Vectors

In some embodiments, compositions and systems provided herein comprise a lipid particle. In some embodiments, a lipid particle is a lipid nanoparticle (LNP). In some embodiments, a lipid or a lipid nanoparticle can encapsulate an expression vector as described herein. LNPs are a non-viral delivery system for delivery of the composition and/or system components described herein. LNPs are particularly effective for delivery of nucleic acids. Beneficial properties of LNP include case of manufacture, low cytotoxicity and immunogenicity, high efficiency of nucleic acid encapsulation and cell transfection, multi-dosing capabilities and flexibility of design (Kulkarni et al., (2018) Nucleic Acid Therapeutics, 28 (3): 146-157). In some embodiments, compositions and methods comprise a lipid, polymer, nanoparticle, or a combination thereof, or use thereof, to introduce one or more effector proteins, one or more guide nucleic acids, one or more donor nucleic acids, or any combinations thereof to a cell. Non-limiting examples of lipids and polymers are cationic polymers, cationic lipids, ionizable lipids, or bio-responsive polymers. In some embodiments, the ionizable lipids exploits chemical-physical properties of the endosomal environment (e.g., pH) offering improved delivery of nucleic acids. In some embodiments, the ionizable lipids are neutral at physiological pH. In some embodiments, the ionizable lipids are protonated under acidic pH. In some embodiments, the bio-responsive polymer exploits chemical-physical properties of the endosomal environment (e.g., pH) to preferentially release the genetic material in the intracellular space.

In some embodiments, a LNP comprises an outer shell and an inner core. In some embodiments, the outer shell comprises lipids. In some embodiments, the lipids comprise modified lipids. In some embodiments, the modified lipids comprise pegylated lipids. In some embodiments, the lipids comprise one or more of cationic lipids, anionic lipids, ionizable lipids, and non-ionic lipids. In some embodiments, the LNP comprises one or more of N1,N3,N5-tris(3-(didodecylamino) propyl) benzene-1,3,5-tricarboxamide (TT3), 2-diolcoyl-sn-glycero-3-phosphoethanolamine (DOPE), 1-palmitoyl-2-olcoylsn-glycero-3-phosphoethanolamine (POPE), 1,2-distearoyl-sn-glycero-3-phosphocholine (DSPC), cholesterol (Chol), 1,2-dimyristoyl-sn-glycerol, and methoxypolyethylene glycol (DMG-PEChooo), derivatives, analogs, or variants thereof. In some embodiments, the LNP has a negative net overall charge prior to complexation with one or more of a guide nucleic acid, a nucleic acid encoding the one or more guide nucleic acid, a nucleic acid encoding the effector protein, and/or a donor nucleic acid. In some embodiments, the inner core is a hydrophobic core. In some embodiments, the one or more of a guide nucleic acid, the one or more nucleic acid encoding the one or more guide nucleic acid, one or more nucleic acid encoding one or more effector protein, and/or the one or more donor nucleic acid forms a complex with one or more of the cationic lipids and the ionizable lipids. In some embodiments, the nucleic acid encoding the effector protein or the nucleic acid encoding the guide nucleic acid is self-replicating.

In some embodiments, a LNP comprises one or more of cationic lipids, ionizable lipids, and modified versions thereof. In some embodiments, the ionizable lipid comprises TT3 or a derivative thereof. Accordingly, in some embodiments, the LNP comprises one or more of TT3 and pegylated TT3. The publication WO2016187531 is hereby incorporated by reference in its entirety, which describes representative LNP formulations in TABLE 2 and TABLE 3, and representative methods of delivering LNP formulations in Example 7.

In some embodiments, a LNP comprises a lipid composition targeting to a specific organ. In some embodiments, the lipid composition comprises lipids having a specific alkyl chain length that controls accumulation of the LNP in the specific organ (e.g., liver or spleen). In some embodiments, the lipid composition comprises a biomimetic lipid that controls accumulation of the LNP in the specific organ (e.g., brain). In some embodiments, the lipid composition comprises lipid derivatives (e.g., cholesterol derivatives) that controls accumulation of the LNP in a specific cell (e.g., liver endothelial cells. Kupffer cells, hepatocytes).

### Viral Vectors and Delivery Thereof

In some embodiments, a vector described herein comprises a viral vector. In some embodiments, the viral vector comprises a nucleic acid to be delivered into a host cell by a recombinantly produced virus or viral particle. The nucleic acid may be single-stranded or double stranded, linear or circular, segmented or non-segmented. The nucleic acid may comprise DNA, RNA, or a combination thereof. An expression vector can be a viral vector. In some embodiments, the expression vector is an adeno-associated viral vector.

There are a variety of viral vectors that are associated with various types of viruses, including but not limited to retroviruses (e.g., lentiviruses and γ-retroviruses), adenoviruses, arenaviruses, alphaviruses, adeno-associated viruses (AAVs), baculoviruses, vaccinia viruses, herpes simplex viruses and poxviruses. In some embodiments, the vector is an adeno-associated viral (AAV) vector. In some embodiments, the viral vector is a recombinant viral vector. In some embodiments, the vector is a retroviral vector. In some embodiments, the retroviral vector is a lentiviral vector. In some embodiments, the retroviral vector comprises gamma-retroviral vector. A viral vector provided herein can be derived from or based on any such virus. For example, in some embodiments, the gamma-retroviral vector is derived from a Moloney Murine Leukemia Virus (MoMLV, MMLV, MuLV, or MLV) or a Murine Stem cell Virus (MSCV) genome. In some embodiments, the lentiviral vector is derived from the human immunodeficiency virus (HIV) genome. In some embodiments, the viral vector is a chimeric viral vector. In some embodiments, the chimeric viral vector comprises viral portions from two or more viruses. In some embodiments, the viral vector corresponds to a virus of a specific serotype.

In some embodiments, a viral vector is an adeno-associated viral vector (AAV vector). In some embodiments, a viral particle that delivers a viral vector described herein is an AAV. In some embodiments, the AAV comprises any AAV known in the art. In some embodiments, the viral vector corresponds to a virus of a specific AAV serotype. In some embodiments, the AAV serotype is selected from an AAV1 serotype, an AAV2 serotype, AAV3 serotype, an AAV4 serotype, AAV5 serotype, an AAV6 serotype. AAV7 serotype, an AAV8 serotype, an AAV9 serotype, an AAV10 serotype, an AAV11 serotype, an AAV12 serotype, an AAV-rh10 serotype, and any combination, derivative, or variant thereof. In some embodiments, the AAV vector is a recombinant vector, a hybrid AAV vector, a chimeric AAV vector, a self-complementary AAV (scAAV) vector, a single-stranded AAV, or any combination thereof, scAAV genomes are generally known in the art and contain both DNA strands which can anneal together to form double-stranded DNA.

In some embodiments, an AAV vector described herein is a chimeric AAV vector. In some embodiments, the chimeric AAV vector comprises an exogenous amino acid or an amino acid substitution, or capsid proteins from two or more serotypes. In some examples, a chimeric AAV vector may be genetically engineered to increase transduction efficiency, selectivity, or a combination thereof.

Often the viral vectors provided herein are an adeno-associated viral vector (AAV vector). Generally, an AAV vector has two inverted terminal repeats (ITRs). According, in some embodiments, the viral vector provided herein comprises two inverted terminal repeats of AAV. The DNA sequence in between the ITRs of an AAV vector provided herein may be referred to herein as the sequence encoding the genome editing tools. These genome editing tools can include, but are not limited to, an effector protein, effector protein modifications (e.g., nuclear localization signal (NLS), enhancer, intron, polyA tail), guide nucleic acid(s), a nucleic acid encoding an effector protein, a nucleic acid encoding a guide nucleic acid, respective promoter(s), and a donor nucleic acid, more than one of the foregoing, or combinations thereof.

In general, viral vectors provided herein comprise at least one promotor or a combination of promoters driving expression or transcription of one or more genome editing tools described herein. In some embodiments, the viral vector comprises two promoters. In some embodiments, the viral vector comprises three promoters. In some embodiments, the length of the promoter is less than about 500, less than about 400, or less than about 300 linked nucleotides. In some embodiments, the length of the promoter is at least 100 linked nucleotides. Non-limiting examples of promoters include CMV, 7SK, EF1a, RPBSA, hPGK, EFS, SV40, PGK1, Ubc, human beta actin promoter, CAG, TRE, UAS, Ac5, Polyhedrin, CaMKIIa, GAL1, H1, TEF1, GDS, ADH1, CaMV35S, Ubi, U6, MNDU3, and MSCV. In some embodiments, the promoter is an inducible promoter that only drives expression of its corresponding gene when a signal is present, e.g., a hormone, a small molecule, a peptide.

In some embodiments, the coding region of the AAV vector forms an intramolecular double-stranded DNA template thereby generating an AAV vector that is a self-complementary AAV (scAAV) vector. In general, the sequence encoding the genome editing tools of an scAAV vector has a length of about 2 kb to about 3 kb. The scAAV vector can comprise nucleotide sequences encoding an effector protein, providing guide nucleic acids described herein, and a donor nucleic acid described herein. In some embodiments, the AAV vector provided herein is a self-inactivating AAV vector.

In some embodiments, an AAV vector provided herein comprises a modification, such as an insertion, deletion, chemical alteration, or synthetic modification, relative to a wild-type AAV vector.

In some embodiments, the viral particle that delivers the viral vector described herein is an AAV, AAVs are characterized by their serotype. Non-limiting examples of AAV serotypes are AAV1, AAV2, AAV3, AAV4, AAV5, AAV6, AAV7, AAV8, AAV9, AAV10, AAV11, AAV12, scAAV, AAV-rh10, chimeric or hybrid AAV, or any combination, derivative, or variant thereof.

### Producing AAV Delivery Vectors

In some embodiments, methods of producing AAV delivery vectors herein comprise packaging a nucleic acid encoding an effector protein and a guide nucleic acid, or a combination thereof, into an AAV vector. In some embodiments, methods of producing the delivery vector comprises. (a) contacting a cell with at least one nucleic acid encoding: (i) a guide nucleic acid; (ii) a Replication (Rep) gene; and (iii) a Capsid (Cap) gene that encodes an AAV capsid protein; (b) expressing the AAV capsid protein in the cell; (c) assembling an AAV particle; and (d) packaging an effector encoding nucleic acid into the AAV particle, thereby generating an AAV delivery vector. In some embodiments, promoters, stuffer sequences, and any combination thereof may be packaged in the AAV vector. In some examples, the AAV vector may package 1, 2, 3, 4, or 5 guide nucleic acids or copies thereof. In some embodiments, the AAV vector comprises inverted terminal repeats, e.g., a 5′ inverted terminal repeat and a 3′ inverted terminal repeat. In some embodiments, the AAV vector comprises a mutated inverted terminal repeat that lacks a terminal resolution site.

In some embodiments, a hybrid AAV vector is produced by transcapsidation, e.g., packaging an inverted terminal repeat (ITR) from a first serotype into a capsid of a second serotype, wherein the first and second serotypes may be not the same. In some examples, the Rep gene and ITR from a first AAV serotype (e.g., AAV2) may be used in a capsid from a second AAV serotype (e.g., AAV9), wherein the first and second AAV serotypes may be not the same. As a non-limiting example, a hybrid AAV serotype comprising the AAV2 ITRs and AAV9 capsid protein may be indicated AAV2/9. In some examples, the hybrid AAV delivery vector comprises an AAV2/1, AAV2/2, AAV2/4, AAV2/5, AAV2/8, or AAV2/9 vector.

### Producing AAV Particles

The AAV particles described herein can be referred to as recombinant AAV (rAAV). Often, rAAV particles are generated by transfecting AAV producing cells with an AAV-containing plasmid carrying the sequence encoding the genome editing tools, a plasmid that carries viral encoding regions, i.e., Rep and Cap gene regions; and a plasmid that provides the helper genes such as EIA, EIB, E2A, E40RF6 and VA. In some embodiments, the AAV producing cells are mammalian cells. In some embodiments, host cells for rAAV viral particle production are mammalian cells. In some embodiments, a mammalian cell for rAAV viral particle production is a COS cell, a HEK293T cell, a HeLa cell, a KB cell, a derivative thereof, or a combination thereof. In some embodiments, rAAV virus particles can be produced in the mammalian cell culture system by providing the rAAV plasmid to the mammalian cell. In some embodiments, producing rAAV virus particles in a mammalian cell can comprise transfecting vectors that express the rep protein, the capsid protein, and the gene-of-interest expression construct flanked by the ITR sequence on the 5′ and 3′ ends. Methods of such processes are provided in, for example. Naso et al., BioDrugs, 2017 August; 31 (4): 317-334 and Benskey et al., (2019), Methods Mol Biol, 1937:3-26, each of which is incorporated by reference in their entireties.

In some embodiments, rAAV is produced in a non-mammalian cell. In some embodiments, rAAV is produced in an insect cell. In some embodiments, an insect cell for producing rAAV viral particles comprises a Sf9 cell. In some embodiments, production of rAAV virus particles in insect cells can comprise baculovirus. In some embodiments, production of rAAV virus particles in insect cells can comprise infecting the insect cells with three recombinant baculoviruses, one carrying the cap gene, one carrying the rep gene, and one carrying the gene-of-interest expression construct enclosed by an ITR on both the 5′ and 3′ end. In some embodiments, rAAV virus particles are produced by the One Bac system. In some embodiments, rAAV virus particles can be produced by the Two Bac system. In some embodiments, in the Two Bac system, the rep gene and the cap gene of the AAV is integrated into one baculovirus virus genome, and the ITR sequence and the gene-of-interest expression construct is integrated into another baculovirus virus genome. In some embodiments, in the One Bac system, an insect cell line that expresses both the rep protein and the capsid protein is established and infected with a baculovirus virus integrated with the ITR sequence and the gene-of-interest expression construct. Details of such processes are provided in, for example. Smith et, al., (1983), Mol. Cell. Biol., 3 (12): 2156-65; Urabe et al., (2002), Hum. Gene. Ther., 1; 13 (16): 1935-43; and Benskey et al., (2019), Methods Mol Biol., 1937:3-26, each of which is incorporated by reference in its entirety.

### V. Target Nucleic Acids

Disclosed herein are compositions, systems and methods for detecting and/or editing a target nucleic acid. In some instances, the target nucleic acid is a single stranded nucleic acid. Alternatively, or in combination, the target nucleic acid is a double stranded nucleic acid and is prepared into single stranded nucleic acids before or upon contacting the reagents. In some instances, the target nucleic acid is a double stranded nucleic acid. In some instances, the double stranded nucleic acid is DNA. The target nucleic acid may be a RNA. The target nucleic acids include but are not limited to mRNA, rRNA, tRNA, non-coding RNA, long non-coding RNA, ssRNA (single stranded RNA), and microRNA (miRNA). In some instances, the target nucleic acid is complementary DNA (cDNA) synthesized from a single-stranded RNA template in a reaction catalyzed by a reverse transcriptase. In some cases, the target nucleic acid is single-stranded RNA (ssRNA) or mRNA. In some cases, the target nucleic acid is from a virus, a parasite, or a bacterium described herein.

In some cases, an effector protein or a multimeric complex thereof recognizes a PAM on a target nucleic acid. In some cases, multiple effector proteins of the multimeric complex recognize a PAM on a target nucleic acid. In some cases, only one effector protein of the multimeric complex recognizes a PAM on a target nucleic acid. In some cases, the PAM is 3′ to the spacer region of the crRNA. In some cases, the PAM is directly 3′ to the spacer region of the crRNA. In some cases, the PAM sequence comprises a sequence listed in TABLE 3.

An effector protein of the present disclosure, a dimer thereof, or a multimeric complex thereof may cleave or nick a target nucleic acid within or near a protospacer adjacent motif (PAM) sequence of the target nucleic acid. In some instances, cleavage occurs within 1, 2, 3, 4, 5, 6, 7, 8, 9 or 10 nucleosides of a 5′ or 3′ terminus of a PAM sequence. A target nucleic acid may comprise a PAM sequence adjacent to a sequence that is complementary to a guide nucleic acid spacer region. In some cases, the PAM sequence is read 5′ to 3′ as set forth in TABLE 3.

In some instances, the effector protein comprises an amino acid sequence that is at least 70%, at least 75%, at least 80%, at least 85%, at least 90%, at least 95%, at least 98%, at least 99%, or 100% identical to any one of the sequences of TABLE 1, and the target nucleic acid comprises a PAM sequence of any one of the sequences as set forth in TABLE 3.

In some instances, the target nucleic acid as described in the methods herein does not initially comprise a PAM sequence. However, any target nucleic acid of interest may be generated using the methods described herein to comprise a PAM sequence, and thus be a PAM target nucleic acid. A PAM target nucleic acid, as used herein, refers to a target nucleic acid that has been amplified to insert a PAM sequence that is recognized by an effector system described herein.

In some cases, the target nucleic acid comprises 5 to 100, 5 to 90, 5 to 80, 5 to 70, 5 to 60, 5 to 50, 5 to 40, 5 to 30, 5 to 25, 5 to 20, 5 to 15, or 5 to 10 linked nucleosides. In some cases, the target nucleic acid comprises 10 to 90, 20 to 80, 30 to 70, or 40 to 60 linked nucleosides. In some cases, the target nucleic acid comprises 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 60, 70, 80, 90, or 100 linked nucleosides. In some instances, the target nucleic acid comprises at least 10, at least 20, at least 30, at least 40, at least 50, at least 60, at least 70, at least 80, at least 90, or at least 100 linked nucleosides. In some embodiments, the target sequence in the target nucleic acid comprises at least 10 contiguous nucleotides that are complementary to the guide nucleic acid or engineered guide nucleic acid.

In some embodiments, compositions, systems, and methods described herein comprise a target nucleic acid may be responsible for a disease, contain a mutation (e.g., single strand polymorphism, point mutation, insertion, or deletion), be contained in an amplicon, or be uniquely identifiable from the surrounding nucleic acids (e.g., contain a unique sequence of nucleotides). In some embodiments, the target nucleic acid has undergone a modification (e.g., an editing) after contacting with an RNP. In some embodiments, the editing is a change in the sequence of the target nucleic acid. In some embodiments, the change comprises an insertion, deletion, or substitution of one or more nucleotides compared to the target nucleic acid that has not undergone any modification.

Duchenne Muscular Dystrophy (DMD) is a severe X-linked recessive neuromuscular disorder effecting approximately 1 in 4,000 live male births. It is caused by mutations in the dystrophin gene (Chromosome X; 31,117,228-33,344,609 (Genome Reference Consortium-GRCh38/hg38)). With a genomic region of over 2.2 megabases in length, dystrophin is the second largest human gene. The dystrophin gene contains 79 exons that are processed into an 11,000 base pair mRNA that is translated into a 427 kDa protein. Functionally, dystrophin acts as a linker between the actin filaments and the extracellular matrix within muscle fibers. The N-terminus of dystrophin is an actin binding domain, while the C-terminus interacts with a transmembrane scaffold that anchors the muscle fiber to the extracellular matrix. Upon muscle contraction, dystrophin provides structural support that allows the muscle tissue to withstand mechanical force. DMD is caused by a wide variety of mutations within the dystrophin gene that result in premature stop codons and therefore a truncated dystrophin protein. Truncated dystrophin proteins do not contain the C-terminus, and therefore cannot provide the structural support necessary to withstand the stress of muscle contraction. As a result, the muscle fibers pull themselves apart, which leads to muscle wasting.

Patients are generally diagnosed by the age of 4, and wheelchair bound by the age of 10. Most patients do not live past the age of 25 due to cardiac and/or respiratory failure. Existing treatments are palliative at best. The most common treatment for DMD is steroids, which are used to slow the loss of muscle strength. However, because most DMD patients start receiving steroids early in life, the treatment delays puberty and further contributes to the patient's diminished quality of life. Thus, there remains a need for compositions, systems and methods for treating disorders associated with the dystrophin gene, such as DMD.

In some embodiments, the target nucleic acid comprises a portion or a specific region of a nucleic acid from a genomic locus, any DNA amplicon of, a reverse transcribed mRNA, or a cDNA from the gene of TABLE 9.

The terms “dystrophin” and “DMD,” as used herein, refers to the dystrophin from any vertebrate source, including mammals such as primates (e.g., humans), dogs, and rodents (e.g., mice and rats), unless otherwise indicated. Dystrophin is a protein which forms a component of the dystrophin-glycoprotein complex (DGC), which bridges the inner cytoskeleton and the extracellular matrix. The gene encoding human dystrophin, referred to as DMD, contains 79 exons and spans 2.4 Mb, and is located on chromosome X, at cytogenetic location Xp21.2-p21.1. An exemplary amino acid sequence of dystrophin. UniProtKB protein P11532 (DMD_HUMAN), is in TABLE 9.1 as SEQ ID NO: 1461.

An exemplary encoding nucleic acid sequence of human dystrophin can be found at NCBI Reference Sequence No. NM_004006.3 and is provided TABLE 9.1.

The genomic locations of dystrophin, isoform-1, exons can be found at Ensembl No. ENST00000357033.9 Human (GRCh38.p13) and is provided, at least in part, in TABLE 9.2 as SEQ ID NO: 1462.

In some embodiments, at least partial sequences of certain exemplary genomic exons can be found in TABLE 9.3 as SEQ ID NOS: 1463-1467.

In some embodiments, the target sequence is within the human dystrophin gene. In some embodiments, the target sequence is within an exon of the human dystrophin gene. In some embodiments, then target sequence covers the junction of two exons. In some embodiments, the target sequence is located within about 1 to about 300 nucleotides, about 10 to about 250, about 20 to about 200, about 30 to about 150, about 40 to about 100, or about 50 nucleotides of the 5′ untranslated region (UTR). In some embodiments, the target sequence is located within about 1 to about 300 nucleotides, about 10 to about 250, about 20 to about 200, about 30 to about 150, about 40 to about 100, or about 50 nucleotides of the 3′ UTR.

In some embodiments, the target sequence is at least partially within a targeted exon within the human dystrophin gene. As used herein the term “targeted exon” can mean any portion within, contiguous with, or adjacent to a specified exon of interest can be targeted by the compositions, systems, and methods described herein. In some embodiments, one or more of exons 1 to exon 79, exon 15 to exon 60, exon 20 to exon 55, exon 40 to exon 55, or exon 44 to exon 53 are targeted. In some embodiments, one or more of exon 44, exon 45, exon 50, exon 51, exon 53, or any combination thereof, of the human dystrophin gene are targeted. Accordingly, in some embodiments: exon 44 is targeted; exon 45 is targeted; exon 50 is targeted; exon 51 is targeted; exon 53 is targeted; or any combination thereof.

In some embodiments, the start of an exon is referred to interchangeably herein as the 5′ end of an exon. In certain embodiments, the 5′ region of an exon comprises a sequence about 1 to about 300 nucleotides adjacent to the 5′ end of an exon when moving upstream in the 5′ direction, or a sequence about 1 to about 300 nucleotides adjacent to the 5′ end of an exon when moving downstream in the 3′ direction, or both.

In some embodiments, the end of an exon is referred to interchangeably herein as the 3′ end of an exon. In certain embodiments, the 3′ region of an exon comprises a sequence about 1 to about 300 nucleotides adjacent to the 3′ end of an exon when moving upstream in the 5′ direction, or a sequence about 1 to about 300 nucleotides adjacent to the 3′ end of an exon when moving downstream in the 3′ direction, or both.

Nucleic acids, such as DNA and pre-mRNA, can contain at least one intron and at least one exon, wherein as read in the 5′ to the 3′ direction of a nucleic acid strand, the 3′ end of an intron can be adjacent to the 5′ end of an exon, and wherein said intron and exon correspond for transcription purposes. If a nucleic acid strand contains more than one intron and exon, the 5′ end of the second intron is adjacent to the 3′ end of the first exon, and 5′ end of the second exon is adjacent to the 3′ end of the second intron. The junction between an intron and an exon can be referred to herein as a splice junction, wherein a 5′ splice site (SS) can refer to the +1/+2 position at the 5′ end of intron and a 3 SS can refer to the last two positions at the 3′ end of an intron. Alternatively, a 5′ SS can refer to the 5′ end of an exon and a 3′SS can refer to the 3′ end of an exon. In certain embodiments, nucleic acids can contain one or more elements that act as a signal during transcription, splicing, and/or translation. In certain embodiments, signaling elements include a 5′SS, a 3′SS, a premature stop codon, U1 and/or U2 binding sequences, and cis acting elements such as branch site (BS), polypyridine tract (PYT), exonic and intronic splicing enhancers (ESEs and ISEs) or silencers (ESSs and ISSs). In some embodiments, nucleic acids may also comprise an untranslated region (UTR), such as a 5′ UTR or a 3′ UTR. In some embodiments, the start of an exon or intron is referred to interchangeably herein as the 5′ end of an exon or intron, respectively. Likewise, in some embodiments, the end of an exon or intron is referred to interchangeably herein as the 3′ end of an exon or intron, respectively.

In some embodiments, at least a portion of at least one target sequence is within about 1, about 5 or more, about 10 or more, about 15 or more, about 20 or more, about 25 or more, about 30 or more, about 35 or more, about 40 or more, about 45 or more, about 50 or more, about 55 or more, about 60 or more, about 65 or more, about 70 or more, about 75 or more, about 80 or more, about 85 or more, about 90 or more, about 95 or more, about 100 or more, about 105 or more, about 110 or more, about 115 or more, about 120 or more, about 125 or more, about 130 or more, about 135 or more, about 140 or more, about 145 or more, or about 150 to about 300 nucleotides adjacent to: the 5′ end of an exon; the 3′ end of an exon; the 5′ end of an intron; the 3′ end of an intron; one or more signaling element comprising a 5′SS, a 3′SS, a premature stop codon, U1 binding sequence, U2 binding sequence, a BS, a PYT, ESE, an ISE, an ESS, an ISS; a 5′ UTR; a 3′ UTR; more than one of the foregoing, or any combination thereof.

In some embodiments, a target sequence that a guide nucleic acid binds is at least partially within a targeted exon within the human dystrophin gene, and wherein at least a portion of the target nucleic acid is within a sequence about 1 to about 300 nucleotides adjacent to: the start of a targeted exon, the end of a targeted exon, or both. In some embodiments, at least a portion of the target sequence that a guide nucleic acid binds can comprise a sequence about 1 to about 300 nucleotides, about 10 to about 250, about 20 to about 200, about 30 to about 150, about 40 to about 100, or about 50 nucleotides adjacent to: the start of a targeted exon, the end of a targeted exon, or both.

In some embodiments, at least a portion of the target nucleic acid that a guide nucleic acid binds is within a sequence about 5 or more, about 10 or more, about 15 or more, about 20 or more, about 25 or more, about 30 or more, about 35 or more, about 40 or more, about 45 or more, about 50 or more, about 55 or more, about 60 or more, about 65 or more, about 70 or more, about 75 or more, about 80 or more, about 85 or more, about 90 or more, about 95 or more, about 100 or more, about 105 or more, about 110 or more, about 115 or more, about 120 or more, about 125 or more, about 130 or more, about 135 or more, about 140 or more, about 145 or more, or about 150 or more nucleotides adjacent to: the start of a targeted exon, the end of a targeted exon, or both.

In some embodiments, a target sequence that a guide nucleic acid binds is at least partially within a targeted exon within the human dystrophin gene, and wherein at least a portion of the target nucleic acid is within a sequence about 1 to about 300 nucleotides adjacent to: the start of a targeted exon, the end of a targeted exon, or both. In some embodiments, at least a portion of the target sequence that a guide nucleic acid binds can comprise a sequence about 1 to about 300 nucleotides, about 10 to about 250, about 20 to about 200, about 30 to about 150, about 40 to about 100, or about 50 nucleotides adjacent to: one or more signaling element comprising a 5′SS, a 3′SS, a premature stop codon, U1 binding sequence, U2 binding sequence, a BS, a PYT, ESE, an ISE, an ESS, an ISS, more than one of the foregoing, or any combination thereof.

In certain embodiments, at least a portion of the target nucleic acid that a guide nucleic acid binds is within a sequence about 5 or more, about 10 or more, about 15 or more, about 20 or more, about 25 or more, about 30 or more, about 35 or more, about 40 or more, about 45 or more, about 50 or more, about 55 or more, about 60 or more, about 65 or more, about 70 or more, about 75 or more, about 80 or more, about 85 or more, about 90 or more, about 95 or more, about 100 or more, about 105 or more, about 110 or more, about 115 or more, about 120 or more, about 125 or more, about 130 or more, about 135 or more, about 140 or more, about 145 or more, or about 150 or more nucleotides adjacent to: one or more signaling element comprising a 5′SS, a 3′SS, a premature stop codon, U1 binding sequence. U2 binding sequence, a BS, a PYT, ESE, an ISE, an ESS, an ISS, more than one of the foregoing, or any combination thereof.

In some embodiments, the target nucleic acid comprises a target locus. In some embodiments, the target nucleic acid comprises more than one target loci. In some embodiments, the target nucleic acid comprises two target loci. Accordingly, in some embodiments, the target nucleic acid can comprise one or more target sequences.

In some embodiments, compositions, systems, and methods described herein comprise an edited target nucleic acid which can describe a target nucleic acid wherein the target nucleic acid has undergone a change, for example, after contact with an effector protein. In some embodiments, the editing is an alteration in the sequence of the target nucleic acid. In some embodiments, the edited target nucleic acid comprises an insertion, deletion, or replacement of one or more nucleotides compared to the unedited target nucleic acid. In some embodiments, the editing is a mutation.

### Mutations

In some embodiments, target nucleic acids comprise a mutation. In some embodiments, a sequence comprising a mutation may be modified to a wildtype sequence with a composition, system or method described herein. In some embodiments, a sequence comprising a mutation may be detected with a composition, system or method described herein. The mutation may be a mutation of 1, 2, 3, 4, 5, 6, 7, 8, 9, 10, 11, 12, 13, 14, 15, 16, 17, 18, 19, 20 or more nucleotides. In some embodiments, a mutation may result in the insertion of at least one amino acid in a protein encoded by the target nucleic acid. In some embodiments, a mutation may result in the deletion of at least one amino acid in a protein encoded by the target nucleic acid. In some embodiments, a mutation may result in the substitution of at least one amino acid in a protein encoded by the target nucleic acid. In some embodiments, a mutation that results in the deletion, insertion, or substitution of one or more amino acids of a protein encoded by the target nucleic acid may result in misfolding of a protein encoded by the target nucleic acid. In some embodiments, a mutation may result in a premature stop codon, thereby resulting in a truncation of the encoded protein.

In some embodiments, a mutation comprises a point mutation or single nucleotide polymorphism (SNP), a chromosomal mutation, a copy number mutation or variation, an insertion-deletion (indel), a frameshift mutation or any combination thereof. A point mutation optionally comprises a substitution, insertion, or deletion. In some embodiments, an indel mutation is an insertion or deletion of one or more nucleotides. In some embodiments, a frameshift mutation occurs when the number of nucleotides in the insertion/deletion is not divisible by three, and it occurs in a protein coding region. In some embodiments, a mutation comprises a chromosomal mutation. A chromosomal mutation can comprise an inversion, a deletion, a duplication, or a translocation. In some embodiments, a mutation comprises a copy number variation. A copy number variation can comprise a gene amplification or an expanding trinucleotide repeat. In some embodiments, guide nucleic acids described herein hybridize to a region of the target nucleic acid comprising the mutation. The mutation may be located in a non-coding region or a coding region of a gene. The mutation may be located in a non-coding region or a coding region of a gene, wherein the gene is a target nucleic acid. A mutation may be in an open reading frame of a target nucleic acid. In some embodiments, guide nucleic acids described herein hybridize to a portion of the target nucleic acid comprising or adjacent to the mutation.

In some embodiments, target nucleic acids comprise a mutation, wherein the mutation is a SNP. The single nucleotide mutation or SNP may be associated with a phenotype of the sample or a phenotype of the organism from which the sample was taken. The SNP, in some embodiments, is associated with altered phenotype from wild type phenotype. The SNP may be a synonymous substitution or a nonsynonymous substitution. The nonsynonymous substitution may be a missense substitution, or a nonsense point mutation. The synonymous substitution may be a silent substitution. The mutation may be a deletion of one or more nucleotides. Often, the single nucleotide mutation. SNP, or deletion is associated with a disease such as a genetic disorder. The mutation, such as a single nucleotide mutation, a SNP, or a deletion, may be encoded in the sequence of a target nucleic acid from the germline of an organism or may be encoded in a target nucleic acid from a diseased cell.

In some embodiments, target nucleic acids comprise a mutation, wherein the mutation is a deletion, insertion, and/or substitution of 1, 2, 3, 4, 5, 6, 7, 8, 9, 10, 11, 12, 13, 14, 15, 16, 17, 18, 19, 20 or more nucleotides. The mutation may be a deletion, insertion, and/or substitution of about 5, about 10, about 15, about 20, about 25, about 30, about 35, about 40, about 45, about 50, about 55, about 60, about 65, about 70, about 75, about 80, about 85, about 90, about 95, about 100, about 200, about 300, about 400, about 500, about 600, about 700, about 800, about 900, or about 1000 nucleotides. The mutation may be a deletion, insertion, and/or substitution of 1 to 5, 5 to 10, 10 to 15, 15 to 20, 20 to 25, 25 to 30, 30 to 35, 35 to 40, 40 to 45, 45 to 50, 50 to 55, 55 to 60, 60 to 65, 65 to 70, 70 to 75, 75 to 80, 80 to 85, 85 to 90, 90 to 95, 95 to 100, 100 to 200, 200 to 300, 300 to 400, 400 to 500, 500 to 600, 600 to 700, 700 to 800, 800 to 900, 900 to 1000, 1 to 50, 1 to 100, 25 to 50, 25 to 100, 50 to 100, 100 to 500, 100 to 1000, or 500 to 1000 nucleotides.

In some cases, the mutation is selected from the mutations listed in TABLE 9.4. In some embodiments, the mutation may be encoded in the sequence of a target nucleic acid from the germline of an organism or may be encoded in a target nucleic acid from a diseased cell. In some embodiments, the target nucleic acid comprises a mutation associated with a disease. In some examples, a mutation associated with a disease refers to a mutation whose presence in a subject indicates that the subject is susceptible to, or suffers from, a disease, disorder, condition, syndrome, or pathological state. In some examples, a mutation associated with a disease, disorder, condition, syndrome or pathological state refers to a mutation which causes, contributes to the development of, or indicates the existence of the disease, disorder, condition, syndrome or pathological state. A mutation associated with a disease may also refer to any mutation which generates transcription or translation products at an abnormal level, or in an abnormal form, in cells affected by a disease relative to a control without the disease. In some embodiments, a mutation associated with a disease, comprises the co-occurrence of a mutation and the phenotype of a disease. The mutation may occur in a gene, wherein transcription or translation products from the gene occur at a significantly abnormal level or in an abnormal form in a cell or subject harboring the mutation as compared to a non-disease control subject not having the mutation.

In some embodiments, a target nucleic acid described herein comprises a mutation associated with a disease, wherein the target nucleic acid is any one of the target nucleic acids set forth in TABLE 9. In some embodiments, the disease, disorder, condition, syndrome or pathological state comprises any one of the diseases, disorders, or pathological states as set forth in TABLE 10.

The mutation may cause a disease. The disease may comprise, at least in part, an inherited disorder, a neurological disorder, or both. The disease may comprise, at least in part, an inherited disorder. The disease may comprise, at least in part, a neurological disorder. In some embodiments, the neurological disorder to a neuromuscular disorder. In some embodiments, the neuromuscular disorder comprises: muscular dystrophy; duchenne muscular dystrophy (DMD); muscular dystrophy, pseudohypertrophic progressive, duchenne type; severe dystrophinopathy, duchenne type; muscular dystrophy duchenne type; becker muscular dystrophy (BMD); muscular dystrophy, pseudohypertrophic progressive, becker type; benign congenital myopathy; benign pseudohypertrophic muscular dystrophy; becker dystrophinopathy; muscular dystrophy pseudohypertrophic progressive, becker type; muscular dystrophy becker type; cardiomyopathy; x-linked dilated cardiomyopathy, type 3B (CMD3B); or dystrophinopathics.

### Detection and Identification of Target Nucleic Acid

In some instances, the target nucleic acid is in a cell. In general, the cell is a human cell. In some instances, the human cell is a: muscle cell, cardiac cell, visceral cell, cardiac muscle cell, smooth muscle cell, cardiomyocyte, nodal cardiac muscle cell, smooth muscle cell, visceral muscle cell, skeletal muscle cell, myocyte, red (or slow) skeletal muscle cell, white (fast) skeletal muscle cell, intermediate skeletal muscle, muscle satellite cell, muscle stem cell, myoblast, muscle progenitor cell, induced pluripotent stem cell (iPS), or a cell derived from an iPS cell, modified to have its gene edited and differentiated into myoblasts, muscle progenitor cells, muscle satellite cells, muscle stem cells, skeletal muscle cells, cardiac muscle cells or smooth muscle cells.

In some embodiments, an effector protein-guide nucleic acid complex may comprise high selectivity for a target sequence. In some embodiments, an RNP comprise a selectivity of at least 200:1, 100:1, 50:1, 20:1, 10:1, or 5:1 for a target nucleic acid over a single nucleotide variant of the target nucleic acid. In some embodiments, an RNP may comprise a selectivity of at least 5:1 for a target nucleic acid over a single nucleotide variant of the target nucleic acid.

By leveraging such effector protein selectivity, some methods described herein may detect a target nucleic acid present in the sample in various concentrations or amounts as a target nucleic acid population. In some embodiments, the method detects at least 2 target nucleic acid populations. In some embodiments, the method detects at least 3, 4, 5, 6, 7, 8, 9, 10, 20, 30, 40, or 50 target nucleic acid populations. In some embodiments, the method detects 3 to 50, 5 to 40, or 10 to 25 target nucleic acid populations. In some embodiments, the method detects at least 2 individual target nucleic acids. In some embodiments, the method detects at least 3, 5, 10, 20, 30, 40, 50, 100, 200, 300, 400, 500, 600, 700, 800, 900, 1000, 2000, 3000, 4000, 5000, 6000, 7000, 8000, 9000, or 10000 individual target nucleic acids. In some embodiments, the method detects 1 to 10,000, 100 to 8000, 400 to 6000, 500 to 5000, 1000 to 4000, or 2000 to 3000 individual target nucleic acids. In some embodiments, the method detects target nucleic acid present at least at one copy per 10 non-target nucleic acids, 10² non-target nucleic acids, 10³ non-target nucleic acids, 10⁴ non-target nucleic acids, 10⁵ non-target nucleic acids, 10⁶ non-target nucleic acids, 10⁷ non-target nucleic acids, 10⁸ non-target nucleic acids, 10⁹ non-target nucleic acids, or 10¹⁰ non-target nucleic acids.

In some embodiments, compositions described herein exhibit indiscriminate transcleavage of a nucleic acid (e.g., a ssDNA and ssRNA), enabling their use for detection of a nucleic acid (e.g., DNA and RNA) in samples. In some embodiments, target nucleic acids are generated from many nucleic acid templates (e.g., RNA) in order to achieve cleavage of a reporter (e.g., a FQ reporter) in a device (e.g., a DETECTR platform). Certain effector proteins may be activated by a nucleic acid (e.g., a ssDNA and ssRNA), upon which they may exhibit trans cleavage of the nucleic acid (e.g., ssDNA and ssRNA) and may, thereby, be used to cleave the reporter molecules (e.g., ssDNA and ssRNA FQ reporter molecules) in a device (e.g., a DETECTR system). These effector proteins may target nucleic acids present in the sample or nucleic acids generated and/or amplified from any number of nucleic acid templates (e.g., RNA). Described herein are reagents comprising a single stranded reporter nucleic acid comprising a detection moiety, wherein the reporter nucleic acid (e.g., a ssDNA-FQ reporter described herein) is capable of being cleaved by the effector protein, upon generation (e.g., cDNA) and amplification of nucleic acids from a nucleic acid template (e.g., ssRNA) using the methods disclosed herein, thereby generating a first detectable signal. While DNA is used as an exemplary reporter in the foregoing, any suitable reporter may be used.

In some embodiments, a target nucleic acid is an amplified nucleic acid of interest. In some embodiments, the nucleic acid of interest is any nucleic acid disclosed herein or from any sample as disclosed herein. In some embodiments, the nucleic acid of interest is DNA. In some embodiments, the nucleic acid of interest is an RNA. In some embodiments, the nucleic acid of interest is an RNA that is reverse transcribed before amplification. In some embodiments, the target nucleic acid is an amplicon of a target nucleic acid (DNA or RNA) generated via amplification (with or without reverse transcription). In some embodiments, the target nucleic acid is an amplicon of a target nucleic acid (DNA or RNA) generated via amplification that is reverse transcribed before amplification.

In some embodiments, target nucleic acids may activate an effector protein to initiate sequence-independent cleavage of a nucleic acid-based reporter (e.g., a reporter comprising an RNA sequence, or a reporter comprising DNA and RNA). For example, an effector protein of the present disclosure is activated by a target nucleic acid to cleave reporters having an RNA (also referred to herein as an “RNA reporter”). Alternatively, an effector protein of the present disclosure is activated by a target nucleic acid to cleave reporters having an RNA. Alternatively, an effector protein of the present disclosure is activated by a target RNA to cleave reporters having an RNA (also referred to herein as a “RNA reporter”). The RNA reporter may comprise a single-stranded RNA labelled with a detection moiety or may be any RNA reporter as disclosed herein.

Further description of editing or detecting a target nucleic acid in the foregoing genes can be found in more detail in Kim et al., “Enhancement of target specificity of CRISPR-Cas12a by using a chimeric DNA-RNA guide”, Nucleic Acids Res. 2020 Sep. 4; 48 (15): 8601-8616; Wang et al., “Specificity profiling of CRISPR system reveals greatly enhanced off-target gene editing”, Scientific Reports volume 10, Article number: 2269 (2020); Tuladhar et al., “CRISPR-Cas9-based mutagenesis frequently provokes on-target mRNA misregulation”, Nature Communications volume 10, Article number: 4056 (2019); Dong et al., “Genome-Wide Off-Target Analysis in CRISPR-Cas9 Modified Mice and Their Offspring”, G3, Volume 9, Issue 11, 1 Nov. 2019, Pages 3645-3651; Winter et al., “Genome-wide CRISPR screen reveals novel host factors required for Staphylococcus aureus α-hemolysin-mediated toxicity”, Scientific Reports volume 6, Article number: 24242 (2016); and Ma et al., “A CRISPR-Based Screen Identifies Genes Essential for West-Nile-Virus-Induced Cell Death”, Cell Rep, 2015 Jul. 28; 12 (4): 673-83, which are hereby incorporated by reference in their entirety.

### Certain Samples

Various sample types comprising a target nucleic acid of interest are consistent with the present disclosure. These samples may comprise a target nucleic acid for detection. In some instances, the detection of the target nucleic indicates an ailment, such as a disease, or genetic disorder, or genetic information, such as for phenotyping, genotyping, or determining ancestry and are compatible with the reagents and support mediums as described herein. Generally, a sample from an individual or an animal or an environmental sample may be obtained to test for presence of a disease, genetic disorder, or any mutation of interest.

In some embodiments, a sample comprises a target nucleic acid from 0.05% to 20% of total nucleic acids in the sample. In some embodiments, the target nucleic acid is 0.1% to 10% of the total nucleic acids in the sample. In some embodiments, the target nucleic acid is 0.1% to 5% of the total nucleic acids in the sample. In some embodiments, the target nucleic acid is 0.1% to 1% of the total nucleic acids in the sample. In some embodiments, the target nucleic acid is in any amount less than 100% of the total nucleic acids in the sample. In some embodiments, the target nucleic acid is 100% of the total nucleic acids in the sample. In some embodiments, the sample comprises a portion of the target nucleic acid and at least one nucleic acid comprising less than 100% sequence identity to the portion of the target nucleic acid but no less than 50% sequence identity to the portion of the target nucleic acid. For example, the portion of the target nucleic acid comprises a mutation as compared to at least one nucleic acid comprising less than 100% sequence identity to the portion of the target nucleic acid but no less than 50% sequence identity to the portion of the target nucleic acid. In some embodiments, the portion of the target nucleic acid comprises a single nucleotide mutation as compared to at least one nucleic acid comprising less than 100% sequence identity to the portion of the target nucleic acid but no less than 50% sequence identity to the portion of the target nucleic acid.

In some embodiments, a sample comprises target nucleic acid populations at different concentrations or amounts. In some embodiments, the sample has at least 2 target nucleic acid populations. In some embodiments, the sample has at least 3, 4, 5, 6, 7, 8, 9, 10, 20, 30, 40, or 50 target nucleic acid populations. In some embodiments, the sample has 3 to 50, 5 to 40, or 10 to 25 target nucleic acid populations.

In some embodiments, a sample has at least 2 individual target nucleic acids. In some embodiments, the sample has at least 3, 5, 10, 20, 30, 40, 50, 100, 200, 300, 400, 500, 600, 700, 800, 900, 1000, 2000, 3000, 4000, 5000, 6000, 7000, 8000, 9000, or 10000 individual target nucleic acids. In some embodiments, the sample comprises 1 to 10,000, 100 to 8000, 400 to 6000, 500 to 5000, 1000 to 4000, or 2000 to 3000 individual target nucleic acids.

In some embodiments, a sample comprises one copy of target nucleic acid per 10 non-target nucleic acids, 10² non-target nucleic acids, 10³ non-target nucleic acids, 10⁴ non-target nucleic acids, 10⁵ non-target nucleic acids, 10⁶ non-target nucleic acids, 10⁷ non-target nucleic acids, 10⁸ non-target nucleic acids, 10⁹ non-target nucleic acids, or 10¹⁰ non-target nucleic acids.

In some embodiments, samples comprise a target nucleic acid at a concentration of less than 1 nM, less than 2 nM, less than 3 nM, less than 4 nM, less than 5 nM, less than 6 nM, less than 7 nM, less than 8 nM, less than 9 nM, less than 10 nM, less than 20 nM, less than 30 nM, less than 40 nM, less than 50 nM, less than 60 nM, less than 70 nM, less than 80 nM, less than 90 nM, less than 100 nM, less than 200 nM, less than 300 nM, less than 400 nM, less than 500 nM, less than 600 nM, less than 700 nM, less than 800 nM, less than 900 nM, less than 1 μM, less than 2 μM, less than 3 μM, less than 4 μM, less than 5 μM, less than 6 μM, less than 7 μM, less than 8 μM, less than 9 μM, less than 10 μM, less than 100 μM, or less than 1 mM. In some embodiments, the sample comprises a target nucleic acid at a concentration of 1 nM to 2 nM, 2 nM to 3 nM, 3 nM to 4 nM, 4 nM to 5 nM, 5 nM to 6 nM, 6 nM to 7 nM, 7 nM to 8 nM, 8 nM to 9 nM, 9 nM to 10 nM, 10 nM to 20 nM, 20 nM to 30 nM, 30 nM to 40 nM, 40 nM to 50 nM, 50 nM to 60 nM, 60 nM to 70 nM, 70 nM to 80 nM, 80 nM to 90 nM, 90 nM to 100 nM, 100 nM to 200 nM, 200 nM to 300 nM, 300 nM to 400 nM, 400 nM to 500 nM, 500 nM to 600 nM, 600 nM to 700 nM, 700 nM to 800 nM, 800 nM to 900 nM, 900 nM to 1 μM, 1 μM to 2 μM, 2 μM to 3 μM, 3 μM to 4 μM, 4 μM to 5 μM, 5 μM to 6 μM, 6 μM to 7 μM, 7 μM to 8 μM, 8 μM to 9 μM, 9 μM to 10 μM, 10 μM to 100 μM, 100 μM to 1 mM, 1 nM to 10 nM, 1 nM to 100 nM, 1 nM to 1 μM, 1 nM to 10 μM, 1 nM to 100 μM, 1 nM to 1 mM, 10 nM to 100 nM, 10 nM to 1 μM, 10 nM to 10 μM, 10 nM to 100 μM, 10 nM to 1 mM, 100 nM to 1 μM, 100 nM to 10 μM, 100 nM to 100 μM, 100 nM to 1 mM, 1 μM to 10 μM, 1 μM to 100 μM, 1 μM to 1 mM, 10 μM to 100 μM, 10 μM to 1 mM, or 100 μM to 1 mM. In some embodiments, the sample comprises a target nucleic acid at a concentration of 20 nM to 200 μM, 50 nM to 100 μM, 200 nM to 50 μM, 500 nM to 20 μM, or 2 μM to 10 μM. In some embodiments, the target nucleic acid is not present in the sample.

In some embodiments, samples comprise fewer than 10 copies, fewer than 100 copies, fewer than 1000 copies, fewer than 10,000 copies, fewer than 100,000 copies, or fewer than 1,000,000 copies of a target nucleic acid. In some embodiments, the sample comprises 10 copies to 100 copies, 100 copies to 1000 copies, 1000 copies to 10,000 copies, 10,000 copies to 100,000 copies, 100,000 copies to 1,000,000 copies, 10 copies to 1000 copies, 10 copies to 10,000 copies, 10 copies to 100,000 copies, 10 copies to 1,000,000 copies, 100 copies to 10,000 copies, 100 copies to 100,000 copies, 100 copies to 1,000,000 copies, 1,000 copies to 100,000 copies, or 1,000 copies to 1,000,000 copies of a target nucleic acid. In some embodiments, the sample comprises 10 copies to 500,000 copies, 200 copies to 200,000 copies, 500 copies to 100,000 copies, 1000 copies to 50.000 copies, 2000 copies to 20.000 copies, 3000 copies to 10,000 copies, or 4000 copies to 8000 copies. In some embodiments, the target nucleic acid is not present in the sample.

In some instances, the sample is a biological sample, an environmental sample, or a combination thereof. Non-limiting examples of biological samples are blood, serum, plasma, saliva, urine, mucosal sample, peritoneal sample, cerebrospinal fluid, gastric secretions, nasal secretions, sputum, pharyngeal exudates, urethral or vaginal secretions, an exudate, an effusion, and a tissue sample (e.g., a biopsy sample). A tissue sample from a subject may be dissociated or liquified prior to application to detection system of the present disclosure. Non-limiting examples of environmental samples are soil, air, or water. In some instances, an environmental sample is taken as a swab from a surface of interest or taken directly from the surface of interest.

In some instances, the sample is a raw (unprocessed, unedited, unmodified) sample. Raw samples may be applied to a system for detecting or modifying a target nucleic acid, such as those described herein. In some instances, the sample is diluted with a buffer or a fluid or concentrated prior to its application to the system or be applied neat to the detection system. Sometimes, the sample contains no more 20 μl of buffer or fluid. The sample, in some cases, is contained in no more than 1, 5, 10, 15, 20, 25, 30, 35 40, 45, 50, 55, 60, 65, 70, 75, 80, 90, 100, 200, 300, 400, 500 μl, or any of value 1 μl to 500 μl, preferably 10 μL to 200 μL, or more preferably 50 μL to 100 μL of buffer or fluid. Sometimes, the sample is contained in more than 500 μl. In some embodiments, the systems, devices, kits, and methods disclosed herein are compatible with the buffers or fluid disclosed herein.

In some instances, the sample is taken from a human. The sample may comprise one or more cells. The sample may be a tissue sample, e.g., a biopsy sample. In some instances, the cell is a muscle cell. The sample comprises nucleic acids from a cell lysate from a muscle cell, the sample comprises nucleic acids from a cell lysate from a cardiac muscle cell, smooth or visceral muscle cell, or a skeletal muscle cell. In some cases, the sample comprises nucleic acids expressed from a cell.

In some instances, samples are used for diagnosing a disease. In some instances, the disease is a genetic disorder. The sample used for genetic testing may comprise at least one target nucleic acid that may bind to a guide nucleic acid of the reagents described herein. The target nucleic acid, in some cases, comprises a portion of a gene comprising a mutation associated with a genetic disease or a gene whose expression is associated with a genetic disease. Sometimes, the target nucleic acid encodes a disease biomarker, such as a gene mutation. In some embodiments, the target nucleic acid is a portion of a nucleic acid from a genomic locus, any DNA amplicon of, a reverse transcribed mRNA, or a cDNA from a locus of at least one of the genes set forth in TABLE 9. Any region of the aforementioned gene loci may be probed for a mutation or deletion using the compositions and methods disclosed herein. For example, in the DMD gene locus, the compositions and methods for detection disclosed herein may be used to detect a single nucleotide polymorphism or a deletion. In some embodiments, the gene is DMD. In some embodiments, the contacting occurs in vitro. In some embodiments, the contacting occurs in vivo. In some embodiments, the contacting occurs ex vivo. In some embodiments, the target nucleic acid comprises a portion of a nucleic acid from a genomic locus, any DNA amplicon of, a reverse transcribed mRNA, or a cDNA from a locus of DMD.

In some embodiments, the genetic disorder is Duchenne muscular dystrophy. Becker Muscular Disorder, or type 3B dilated cardiomyopathy. The target nucleic acid, in some embodiments, is from a gene with a mutation associated with a genetic disorder, from a gene whose overexpression is associated with a genetic disorder, from a gene associated with abnormal cellular growth resulting in a genetic disorder, or from a gene associated with abnormal cellular metabolism resulting in a genetic disorder. In some embodiments, the target nucleic acid is a nucleic acid from a genomic locus, a transcribed mRNA, or a reverse transcribed mRNA, a DNA amplicon of or a cDNA from a locus of at least one of a gene set forth in TABLE 9. In some embodiments, the target nucleic acid is encoded by a gene described in TABLE 9. In some embodiments, the target nucleic acid is encoded by a gene described in TABLE 9 comprising a mutation. In some embodiments, the target nucleic acid is encoded by a gene described in TABLE 9 comprising a mutation described in TABLE 9.4.

The sample used for phenotyping testing may comprise at least one target nucleic acid that may bind to a guide nucleic acid of the reagents described herein. The target nucleic acid, in some cases, is a nucleic acid encoding a sequence associated with a phenotypic trait. The sample used for genotyping testing may comprise at least one target nucleic acid that may bind to a guide nucleic acid of the reagents described herein. The target nucleic acid, in some cases, is a nucleic acid encoding a sequence associated with a genotype of interest. The sample used for ancestral testing may comprise at least one target nucleic acid that may bind to a guide nucleic acid of the reagents described herein. The target nucleic acid, in some cases, is a nucleic acid encoding a sequence associated with a geographic region of origin or ethnic group.

The sample may be used for identifying a disease status. For example, a sample is any sample described herein, and is obtained from a subject for use in identifying a disease status of a subject. The disease may be a cancer or genetic disorder. Sometimes, a method comprises obtaining a serum sample from a subject; and identifying a disease status of the subject. Often, the disease status is prostate disease status, but the status of any disease may be assessed.

Any of the above disclosed samples are consistent with the methods, compositions, reagents, enzymes, and systems disclosed herein.

### VI. Compositions

Disclosed herein are compositions comprising one or more effector proteins described herein or nucleic acids encoding the one or more effector proteins, one or more guide nucleic acids described herein or nucleic acids encoding the one or more guide nucleic acids described herein, or combinations thereof. In some embodiments, one or more of a repeat sequence, a handle sequence, and intermediary sequence of the one or more guide nucleic acids are capable of interacting with the one or more of the effector proteins. In some embodiments, spacer sequences of the one or more guide nucleic acids hybridizes with a target sequence of a target nucleic acid. In some embodiments, the compositions comprise one or more donor nucleic acids described herein. In some embodiments, the compositions are capable of editing a target nucleic acid in a cell or a subject. In some embodiments, the compositions are capable of editing a target nucleic acid or the expression thereof in a cell, in a tissue, in an organ, in vitro, in vivo, or ex vivo. In some embodiments, the compositions are capable of editing a target nucleic acid in a sample comprising the target nucleic.

In some embodiments, compositions described herein comprise plasmids described herein, viral vectors described herein, non-viral vectors described herein, or combinations thereof. In some embodiments, compositions described herein comprise the viral vectors. In some embodiments, compositions described herein comprise an AAV. In some embodiments, compositions described herein comprise liposomes (e.g., cationic lipids or neutral lipids), dendrimers, lipid nanoparticle (LNP), or cell-penetrating peptides. In some embodiments, compositions described herein comprise an LNP.

### Pharmaceutical Compositions and Modes of Administration

Disclosed herein, in some aspects, are pharmaceutical compositions for modifying a target nucleic acid in a cell or a subject, comprising any one of the effector proteins, engineered effector proteins, fusion effector proteins, or guide nucleic acids as described herein and any combination thereof. Also disclosed herein, in some aspects, are pharmaceutical compositions comprising a nucleic acid encoding any one of the effector proteins, engineered effector proteins, fusion effector proteins, or guide nucleic acids as described herein and any combination thereof. In some embodiments, pharmaceutical compositions comprise a plurality of guide nucleic acids. Pharmaceutical compositions may be used to modify a target nucleic acid or the expression thereof in a cell in vitro, in vivo or ex vivo.

In some embodiments, pharmaceutical compositions comprise one or more nucleic acids encoding an effector protein, fusion effector protein, fusion partner, a guide nucleic acid, or a combination thereof; and a pharmaceutically acceptable carrier or diluent. The effector protein, fusion effector protein, fusion partner protein, or combination thereof may be any one of those described herein. The one or more nucleic acids may comprise a plasmid. The one or more nucleic acids may comprise a nucleic acid expression vector. The one or more nucleic acids may comprise a viral vector. In some embodiments, the viral vector is a lentiviral vector. In some embodiments, the vector is an adeno-associated viral (AAV) vector. In some embodiments, compositions, including pharmaceutical compositions, comprise a viral vector encoding a fusion effector protein and a guide nucleic acid, wherein at least a portion of the guide nucleic acid binds to the effector protein of the fusion effector protein.

In some embodiments, pharmaceutical compositions comprise a virus comprising a viral vector encoding a fusion effector protein, an effector protein, a fusion partner, a guide nucleic acid, or a combination thereof; and a pharmaceutically acceptable carrier or diluent. The virus may be a lentivirus. The virus may be an adenovirus. The virus may be a non-replicating virus. The virus may be an adeno-associated virus (AAV). The viral vector may be a retroviral vector. Retroviral vectors may include gamma-retroviral vectors such as vectors derived from the Moloney Murine Leukemia Virus (MoMLV, MMLV, MuLV, or MLV) or the Murine Stem cell Virus (MSCV) genome. Retroviral vectors may include lentiviral vectors such as those derived from the human immunodeficiency virus (HIV) genome. In some embodiments, the viral vector is a chimeric viral vector, comprising viral portions from two or more viruses. In some embodiments, the viral vector is a recombinant viral vector.

In some embodiments, the viral vector is an AAV. The AAV may be any AAV known in the art. In some embodiments, the viral vector corresponds to a virus of a specific serotype. In some examples, the serotype is selected from an AAV1 serotype, an AAV2 serotype, AAV3 serotype, an AAV4 serotype, AAV5 serotype, an AAV6 serotype, AAV7 serotype, an AAV8 serotype, an AAV9 serotype, an AAV10 serotype, an AAV11 serotype, and an AAV12 serotype. In some embodiments the AAV vector is a recombinant vector, a hybrid AAV vector, a chimeric AAV vector, a self-complementary AAV (scAAV) vector, a single-stranded AAV (ssAAV) or any combination thereof, scAAV genomes are generally known in the art and contain both DNA strands which can anncal together to form double-stranded DNA.

In some embodiments, methods of producing delivery vectors herein comprise packaging a nucleic acid encoding an effector protein and a guide nucleic acid, or a combination thereof, into an AAV vector. In some embodiments, methods of producing the delivery vector comprises. (a) contacting a cell with at least one nucleic acid encoding: (i) a guide nucleic acid; (ii) a Replication (Rep) gene; and (iii) a Capsid (Cap) gene that encodes an AAV capsid protein; (b) expressing the AAV capsid protein in the cell; (c) assembling an AAV particle; and (d) packaging a Cas effector encoding nucleic acid into the AAV particle, thereby generating an AAV delivery vector. In some embodiments, promoters, stuffer sequences, and any combination thereof may be packaged in the AAV vector. In some embodiments.

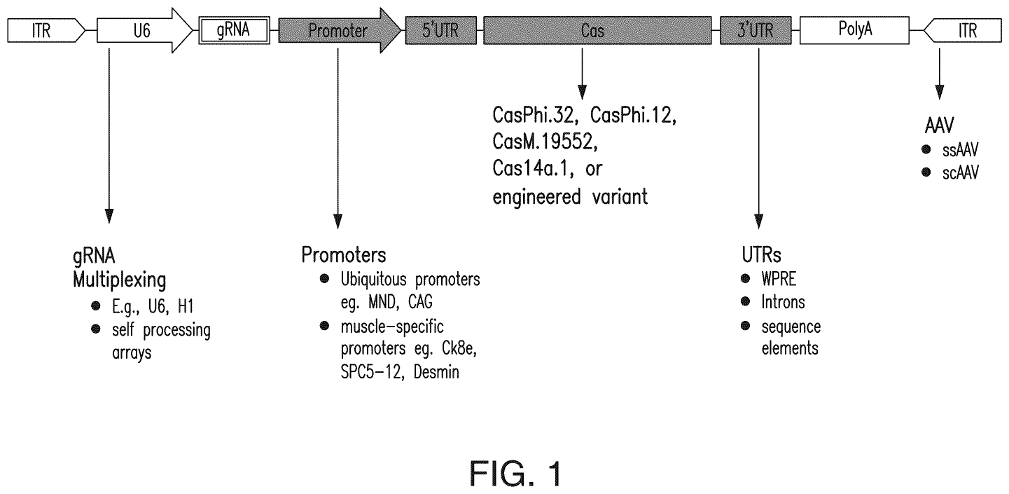

the AAV vector comprises a sequence encoding a guide nucleic acid. In some embodiments, the guide nucleic acid comprises a crRNA. In some embodiments, the guide nucleic acid is a crRNA. In some examples, the AAV vector can package 1, 2, 3, 4, or 5 nucleotide sequences encoding guide nucleic acids or copies thereof. In some examples, the AAV vector packages 1 or 2 nucleotide sequences encoding guide nucleic acids or copies thereof. In some embodiments, the AAV vector packages a nucleotide sequences encoding a first guide nucleic acid and nucleotide sequences encoding a second guide nucleic acid, wherein the first guide nucleic acid and the second guide nucleic acid are the same. In some embodiments, the AAV vector packages a first guide nucleic acid and a second guide nucleic acid, wherein the first guide nucleic acid and the second guide nucleic acid are different. In some embodiments, the AAV vector comprises inverted terminal repeats, e.g., a 5′ inverted terminal repeat and a 3′ inverted terminal repeat. In some embodiments, the inverted terminal repeat comprises inverted terminal repeats from AAV. In some embodiments, the inverted terminal repeat comprises inverted terminal repeats from ssAAV or scAAV. In some embodiments, the AAV vector comprises a mutated inverted terminal repeat that lacks a terminal resolution site. FIG. 1 illustrates an exemplary schematic of AAV construct.

In some embodiments, a hybrid AAV vector is produced by transcapsidation, e.g., packaging an inverted terminal repeat (ITR) from a first serotype into a capsid of a second serotype, wherein the first and second serotypes may be not the same. In some examples, the Rep gene and ITR from a first AAV serotype (e.g., AAV2) may be used in a capsid from a second AAV serotype (e.g., AAV9), wherein the first and second AAV serotypes may be not the same. As a non-limiting example, a hybrid AAV serotype comprising the AAV2 ITRs and AAV9 capsid protein may be indicated AAV2/9. In some examples, the hybrid AAV delivery vector comprises an AAV2/1, AAV2/2, AAV2/4, AAV2/5, AAV2/8, or AAV2/9 vector.

In some embodiments, the AAV vector may be a chimeric AAV vector. In some embodiments, the chimeric AAV vector comprises an exogenous amino acid or an amino acid substitution, or capsid proteins from two or more serotypes. In some examples, a chimeric AAV vector may be genetically engineered to increase transduction efficiency, selectivity, or a combination thereof.

In some examples, the delivery vector may be a eukaryotic vector, a prokaryotic vector (e.g., a bacterial vector) a viral vector, or any combination thereof. In some embodiments, the delivery vehicle may be a non-viral vector. In some embodiments, the delivery vehicle may be a plasmid. In some embodiments, the plasmid comprises DNA. In some embodiments, the plasmid comprises RNA. In some examples, the plasmid comprises circular double-stranded DNA. In some examples, the plasmid may be linear. In some examples, the plasmid comprises one or more genes of interest and one or more regulatory elements. In some examples, the plasmid comprises a bacterial backbone containing an origin of replication and an antibiotic resistance gene or other selectable marker for plasmid amplification in bacteria. In some examples, the plasmid may be a minicircle plasmid. In some examples, the plasmid contains one or more genes that provide a selective marker to induce a target cell to retain the plasmid. In some examples, the plasmid may be formulated for delivery through injection by a needle carrying syringe. In some examples, the plasmid may be formulated for delivery via electroporation. In some examples, the plasmids may be engineered through synthetic or other suitable means known in the art. For example, in some cases, the genetic elements may be assembled by restriction digest of the desired genetic sequence from a donor plasmid or organism to produce ends of the DNA which may then be readily ligated to another genetic sequence.

In some embodiments, the vector is a non-viral vector, and a physical method or a chemical method is employed for delivery into the somatic cell. Exemplary physical methods include electroporation, gene gun, sonoporation, magnetofection, or hydrodynamic delivery. Exemplary chemical methods include delivery of the recombinant polynucleotide via liposomes such as, cationic lipids or neutral lipids; dendrimers; nanoparticles; or cell-penetrating peptides.

In some embodiments, a fusion effector protein as described herein is inserted into a vector. In some embodiments, the vector comprises one or more promoters, enhancers, ribosome binding sites. RNA splice sites, polyadenylation sites, a replication origin, and/or transcriptional terminator sequences.

In some embodiments, the AAV vector comprises a self-processing array system for guide nucleic acid. Such a self-processing array system refers to a system for multiplexing, stringing together multiple guide nucleic acids under the control of a single promoter. In general, plasmids and vectors described herein comprise at least one promoter. In some embodiments, the promoters are constitutive promoters. In other embodiments, the promoters are inducible promoters. In additional embodiments, the promoters are prokaryotic promoters (e.g., drive expression of a gene in a prokaryotic cell). In some embodiments, the promoters are eukaryotic promoters. (e.g., drive expression of a gene in a eukaryotic cell). Exemplary promoters include, but are not limited to, CMV, EF1a, SV40, PGK1, Ubc, human beta actin, CAG, TRE, UAS, Ac5, polyhedron, CaMKIIa, GAL1-10, TEF1, GDS, ADH1, CaMV35S, Ubi, H1, U6, CaMV35S, SV40, CMV, 7SK, and HSV TK promoter. In some embodiments, the promoter is CMV. In some embodiments, the promoter is EF1a. In some embodiments, the promoter is U6. In some embodiments, the promote is H1. In some embodiments, the promoter is 7SK. In some embodiments, the promoter is ubiquitin. In some embodiments, vectors are bicistronic or polycistronic vector (e.g., having or involving two or more loci responsible for generating a protein) having an internal ribosome entry site (IRES) is for translation initiation in a cap-independent manner.

In some embodiments, the AAV vector comprises a promoter for expressing effector proteins. In some embodiments, the promoter for expressing effector protein is a site-specific promoter. In some embodiments, the promoter for expressing effector protein is a muscle-specific promoter. In some embodiments, the muscle-specific promoter comprises Ck8e. SPC5-12, or Desmin promoter sequence. In some embodiments, the promoter for expressing effector protein is a ubiquitous promoter. In some embodiments, the ubiquitous promoter comprises MND or CAG promoter sequence.

In some embodiments, the stuffer sequence comprises 5′ untranslated region, 3′ untranslated region or combination thereof. In some embodiments, the 3′-untranslated region comprises an intron. In some embodiments, the 3′-untranslated region comprises one or more sequence elements, such as an intron sequence or an enhancer sequence. In some embodiments, the 3′-untranslated region comprises an enhancer. In some embodiments, vectors comprise an enhancer. Enhancers are nucleotide sequences that have the effect of enhancing promoter activity. In some embodiments, enhancers augment transcription regardless of the orientation of their sequence. In some embodiments, enhancers activate transcription from a distance of several kilo basepairs. Furthermore, enhancers are located optionally upstream or downstream of a gene region to be transcribed, and/or located within the gene, to activate the transcription. Exemplary enhancers include, but are not limited to, WPRE; CMV enhancers; the R—U5′ segment in LTR of HTLV-I (Mol. Cell. Biol., Vol. 8 (1), p. 466-472, 1988); SV40 enhancer; the intron sequence between exons 2 and 3 of rabbit β-globin (Proc. Natl. Acad. Sci. USA., Vol. 78 (3), p. 1527-31, 1981); and the genome region of human growth hormone (J Immunol., Vol. 155 (3), p. 1286-95, 1995). In some embodiments, the enhancer is WPRE.

In some embodiments, the AAV vector comprises one or more polyadenylation (poly A) signal sequences. In some embodiments, the polyadenlyation signal sequence comprises hGH poly A signal sequence.

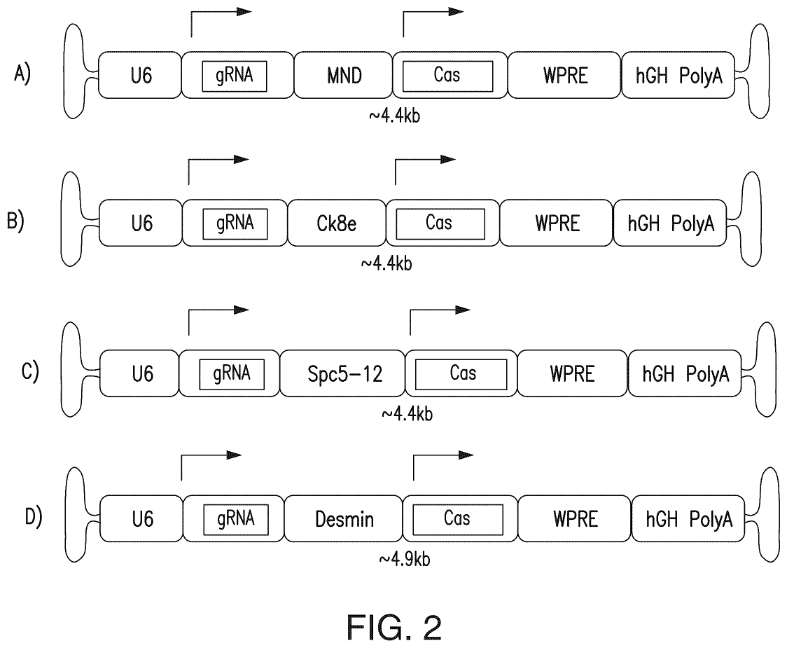

FIG. 2 illustrates exemplary AAV constructs having a nucleic acid encoding one guide nucleic acid. In some embodiments, the guide nucleic acid comprises a guide RNA (e.g., crRNA). Single cutting can be assessed by delivery of a nucleic acid encoding one guide nucleic acid. Accordingly, in some embodiments, any one of AAV construct illustrated in FIG. 2 can be used to modify (e.g., introduce a single cut within or near) a target nucleic acid.

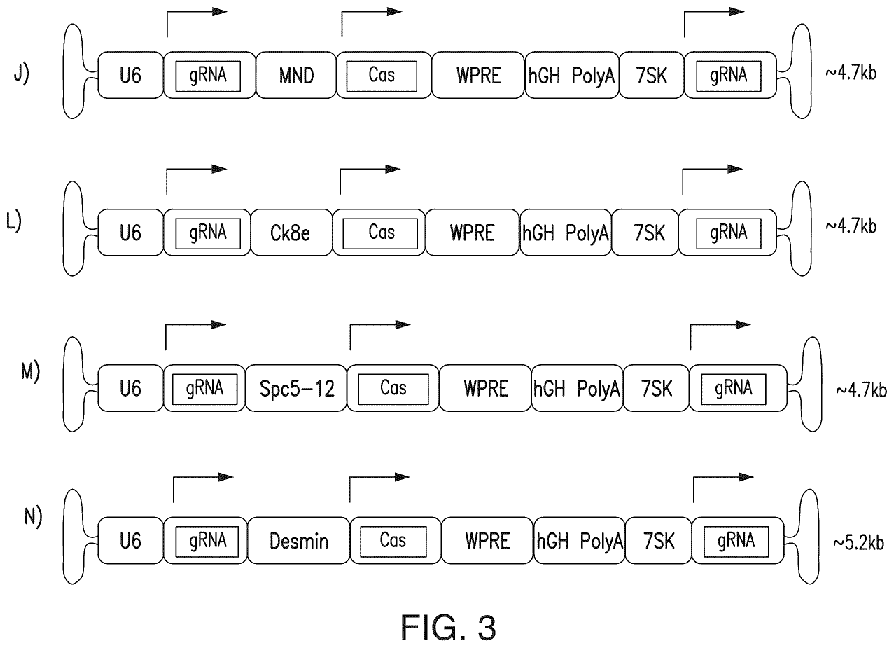

FIG. 3 illustrates exemplary AAV constructs having two nucleic acids encoding two guide nucleic acids. In some embodiments, a first nucleic acid encoding a first guide nucleic acid comprises a first guide RNA and a second nucleic acid encoding a second guide nucleic acid comprises a second guide RNA. In some embodiments, the first nucleic acid and the second nucleic acid are same. Accordingly, in some embodiments, exemplary AAV constructs of FIG. 3 can be used to modify (e.g., introduce a single cut) at a higher rate than the construct having a single copy of a nucleic acid encoding one guide nucleic acid. Alternatively, in some embodiments, the first nucleic acid and the second nucleic acid are different. Dual cutting can be assessed by delivery of two different nucleic acids each encoding guide nucleic acid. Accordingly, in some embodiments, AAV constructs illustrated in FIG. 3 can be used for dual cutting within or near about a target nucleic acid.

Pharmaceutical compositions described herein may comprise a salt. In some embodiments, the salt is a sodium salt. In some embodiments, the salt is a potassium salt. In some embodiments, the salt is a magnesium salt. In some embodiments, the salt is NaCl. In some embodiments, the salt is KNO3. In some embodiments, the salt is Mg2+SO42-.

Non-limiting examples of pharmaceutically acceptable carriers and diluents suitable for the pharmaceutical compositions disclosed herein include buffers (e.g., neutral buffered saline, phosphate buffered saline); carbohydrates (e.g., glucose, mannose, sucrose, dextran, mannitol); polypeptides or amino acids (e.g., glycine); antioxidants; chelating agents (e.g., EDTA, glutathione); adjuvants (e.g., aluminum hydroxide); surfactants (Polysorbate 80. Polysorbate 20, or Pluronic F68); glycerol; sorbitol; mannitol; polyethyleneglycol; and preservatives.

In some embodiments, pharmaceutical compositions are in the form of a solution (e.g., a liquid). The solution may be formulated for injection, e.g., intravenous or subcutaneous injection. In some embodiments, the pH of the solution is about 7, about 7.1, about 7.2, about 7.3, about 7.4, about 7.5, about 7.6, about 7.7, about 7.8, about 7.9, about 8, about 8.1, about 8.2, about 8.3, about 8.4, about 8.5, about 8.6, about 8.7, about 8.8, about 8.9, or about 9. In some embodiments, the pH is 7 to 7.5, 7.5 to 8, 8 to 8.5, 8.5 to 9, or 7 to 8.5. In some cases, the pH of the solution is less than 7. In some cases, the pH is greater than 7.

In some embodiments, pharmaceutical compositions comprise an: effector protein, fusion effector protein, fusion partner, a guide nucleic acid, or a combination thereof; and a pharmaceutically acceptable carrier or diluent. In some embodiments, pharmaceutical compositions comprise one or more nucleic acids encoding an: effector protein, fusion effector protein, fusion partner, a guide nucleic acid, or a combination thereof; and a pharmaceutically acceptable carrier or diluent. In some embodiments, guide nucleic acid can be a plurality of guide nucleic acids. In some embodiments, the effector protein comprises a sequence that is at least 65%, at least 70%, at least 75%, at least 80%, at least 85%, at least 90%, at least 95%, at least 97%, or at least 98%, at least 99%, or 100% identical to any one of the sequences of TABLE 1. In some instances, the guide nucleic acid comprises a nucleobase sequence of any one of the gRNA sequences of TABLE 6. In some instances, the nucleobase sequence of the gRNA is at least 65%, at least 70%, at least 75%, at least 80%, at least 85%, at least 90%, at least 95%, at least 97%, or at least 98%, at least 99%, or 100% identical to any one of the gRNA sequences of TABLE 6.

In combination with a pharmaceutically acceptable carrier or diluent, each row in TABLE 6 can represent an exemplary pharmaceutical composition comprising an effector protein as set forth in TABLE 1 recognizing a PAM sequence as set forth in TABLE 6 and a guide nucleic acid wherein the guide nucleic acid is a gRNA. In some instances, the guide nucleic acid comprises a nucleobase sequence of any one of the gRNA sequences of TABLE 6. In some instances, the nucleobase sequence of the gRNA is at least 65%, at least 70%, at least 75%, at least 80%, at least 85%, at least 90%, at least 95%, at least 97%, or at least 98%, at least 99%, or 100% identical to any one of the gRNA sequences of TABLE 6.

### VII. Systems

Disclosed herein, in some aspects, are systems for detecting, modifying, or editing a target nucleic acid, comprising the effector proteins described herein, or a multimeric complex thereof. In some embodiments, systems comprise one or more components having one or more effector proteins described herein. In some embodiments, systems comprise one or more components having one or more guide nucleic acids described herein. In some embodiments, systems comprise one or more components having a guide nucleic acid and an additional nucleic acid as described herein. Systems may be used to detect, modify, or edit a target nucleic acid. Systems may be used to modify the activity or expression of a target nucleic acid. In some instances, systems comprise an effector protein described herein, a reagent, a support medium, or a combination thereof. In some instances, systems comprise an effector protein described herein, a guide nucleic acid described herein, a reagent, support medium, or a combination thereof. In some embodiments, systems comprise one or more of: an effector protein, a guide nucleic acid, an additional nucleic acid, a reagent, a support medium, or a combination thereof. In some embodiments, systems comprise compositions, a solution, a buffer, a reagent, a support medium, or a combination thereof. In some instances, the effector protein comprises an effector protein, or a fusion protein thereof, described herein.

In some embodiments, effector proteins comprise an amino acid sequence that is at least 70%, at least 75%, at least 80%, at least 85%, at least 90%, at least 95%, or 100% similar to any one of the sequences of TABLE 1. In some embodiments, the amino acid sequence of the effector protein is at least 70%, at least 75%, at least 80%, at least 85%, at least 90%, at least 95%, or 100% similar to any one of the sequences of TABLE 1. In some embodiments, effector protein comprises an amino acid sequence that is at least 70%, at least 75%, at least 80%, at least 85%, at least 90%, at least 95%, or 100% identical to any one of the sequences of TABLE 1. In some embodiments, the amino acid sequence of the effector protein is at least 70%, at least 75%, at least 80%, at least 85%, at least 90%, at least 95%, or 100% identical to any one of the sequences of TABLE 1.

In some instances, the guide nucleic acid comprises a spacer sequence that is at least 70%, at least 75%, at least 80%, at least 85%, at least 90%, at least 92%, at least 95%, or 100% identical to any one of the sequences set forth in TABLE 4 and a repeat sequence that is at least 70%, at least 75%, at least 80%, at least 85%, at least 90%, at least 92%, at least 95%, or 100% identical to the sequence set forth in TABLE 5. In some instances, the nucleotide sequence of the guide nucleic acid is at least 70%, at least 75%, at least 80%, at least 85%, at least 90%, at least 92%, at least 95%, at least 97%, at least 98%, at least 99%, or 100% identical to any one of the gRNA sequences set forth in TABLE 6.

In some embodiments, systems, compositions, methods, kits, devices, and solutions comprise 0.01 μL, 0.02 μL, 0.03 μL, 0.04 μL, 0.05 μL, 0.06 μL, 0.07 μL, 0.08 μL, 0.09 μL, 0.1 μL, 0.2 μL, 0.3 μL, 0.4 μL, 0.5 μL, 0.6 μL, 0.7 μL, 0.8 μL, 0.9 μL, 1 μL, 2 μL, 3 μL, 4 μL, 5 μL, 6 μL, 7 μL, 8 μL, 9 μL, 10 μL, 20 μL, 30 μL, 40 μL, 50 μL, 60 μL, 70 μL, 80 μL, 90 μL, 100 μL, 150 μL, 200 μL, 250 μL, 300 μL, 350 μL, 400 μL, 450 μL, 500 μL, or more, effector proteins, or nucleic acids encoding the effector proteins, as described herein. In some embodiments, systems, compositions, methods, kits, devices, and solutions comprise 1 nM, 2 nM, 3 nM, 4 nM, 5 nM, 6 nM, 7 nM, 8 nM, 9 nM, 10 nM, 20 nM, 30 nM, 40 nM, 50 nM, 60 nM, 70 nM, 80 nM, 90 nM, 100 nM, 150 nM, 200 nM, 250 nM, 300 nM, 350 nM, 400 nM, 450 nM, 500 nM, or more, effector proteins, or nucleic acids encoding the effector proteins, as described herein. In some embodiments, systems, compositions, methods, kits, devices, and solutions comprise 1 μM, 2 μM, 3 μM, 4 μM, 5 μM, 6 μM, 7 μM, 8 μM, 9 μM, 10 μM, 20 μM, 30 μM, 40 μM, 50 μM, 60 μM, 70 μM, 80 μM, 90 μM, 100 μM, 150 μM, 200 μM, 250 μM, 300 μM, 350 μM, 400 μM, 450 μM, 500 μM, or more, effector proteins, or nucleic acids encoding the effector proteins, as described herein. In some embodiments, systems, compositions, methods, kits, devices, and solutions comprise 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, 150 mM, 200 mM, 250 mM, 300 mM, 350 mM, 400 mM, 450 mM, 500 mM, or more, effector proteins, or nucleic acids encoding the effector proteins, as described herein.

In some embodiments, systems, compositions, methods, kits, devices, and solutions comprise 0.01 μL, 0.02 μL, 0.03 μL, 0.04 μL, 0.05 μL, 0.06 μL, 0.07 μL, 0.08 μL, 0.09 μL, 0.1 μL, 0.2 μL, 0.3 μL, 0.4 μL, 0.5 μL, 0.6 μL, 0.7 μL, 0.8 μL, 0.9 μL, 1 μL, 2 μL, 3 μL, 4 μL, 5 μL, 6 μL, 7 μL, 8 μL, 9 μL, 10 μL, 20 μL, 30 μL, 40 μL, 50 μL, 60 μL, 70 μL, 80 μL, 90 μL, 100 μL, 150 μL, 200 μL, 250 μL, 300 μL, 350 μL, 400 μL, 450 μL, 500 μL, or more, guide nucleic acids, or nucleic acids encoding the guide nucleic acids, as described herein. In some embodiments, systems, compositions, methods, kits, devices, and solutions comprise 1 nM, 2 nM, 3 nM, 4 nM, 5 nM, 6 nM, 7 nM, 8 nM, 9 nM, 10 nM, 20 nM, 30 nM, 40 nM, 50 nM, 60 nM, 70 nM, 80 nM, 90 nM, 100 nM, 150 nM, 200 nM, 250 nM, 300 nM, 350 nM, 400 nM, 450 nM, 500 nM, or more, guide nucleic acids, or nucleic acids encoding the guide nucleic acids, as described herein. In some embodiments, systems, compositions, methods, kits, devices, and solutions comprise 1 μM, 2 μM, 3 μM, 4 μM, 5 μM, 6 μM, 7 μM, 8 μM, 9 μM, 10 μM, 20 μM, 30 μM, 40 μM, 50 μM, 60 μM, 70 μM, 80 μM, 90 μM, 100 μM, 150 μM, 200 μM, 250 μM, 300 μM, 350 μM, 400 μM, 450 μM, 500 μM, or more, guide nucleic acids, or nucleic acids encoding the guide nucleic acids, as described herein. In some embodiments, systems, compositions, methods, kits, devices, and solutions comprise 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, 150 mM, 200 mM, 250 mM, 300 mM, 350 mM, 400 mM, 450 mM, 500 mM, or more, guide nucleic acids, or nucleic acids encoding the guide nucleic acids, as described herein.

Systems may be used for detecting the presence or the absence of a target nucleic acid as described herein, for example as set forth in TABLE 9. Systems may be used for detecting the presence or the absence of a mutation of a target nucleic acid as described herein, for example as set forth in TABLE 9.4. Systems may be used for detecting the presence or the absence of a target nucleic acid associated with or causative of a disease or disorder, such as a genetic disorder. Systems may be used for detecting the presence or the absence of a target nucleic acid associated with or causative of a disease or disorder as described herein, for example as set forth in TABLE 10. In some embodiments, systems are useful for phenotyping, genotyping, or determining ancestry. Unless specified otherwise, systems include kits and may be referred to as kits. Unless specified otherwise, systems include devices and may also be referred to as devices. Systems described herein may be provided in the form of a companion diagnostic assay or device, a point-of-care assay or device, or an over-the-counter diagnostic assay/device.

Reagents and effector proteins of various systems may be provided in a reagent chamber or on a support medium. Alternatively, the reagent and/or effector protein may be contacted with the reagent chamber or the support medium by the individual using the system. An exemplary reagent chamber is a test well or container. The opening of the reagent chamber may be large enough to accommodate the support medium. Optionally, the system comprises a buffer and a dropper. The buffer may be provided in a dropper bottle for case of dispensing. The dropper may be disposable and transfer a fixed volume. The dropper may be used to place a sample into the reagent chamber or on the support medium.

### System Solutions

In general, systems and system components comprise a solution in which the activity of an effector protein occurs. Often, the solution comprises or consists essentially of a buffer. The solution or buffer may comprise a buffering agent, a salt, a crowding agent, a detergent, a reducing agent, a competitor, or a combination thereof. Often the buffer is the primary component or the basis for the solution in which the activity occurs. Thus, concentrations for components of buffers described herein (e.g., buffering agents, salts, crowding agents, detergents, reducing agents, and competitors) are the same or essentially the same as the concentration of these components in the solution in which the activity occurs. In some embodiments, a buffer is required for cell lysis activity or viral lysis activity.

In some embodiments, systems comprise a buffer, wherein the buffer comprise at least one buffering agent. Exemplary buffering agents include HEPES, TRIS, MES, ADA, PIPES, ACES, MOPSO, BIS-TRIS propane, BES, MOPS, TES, DISO, Trizma, TRICINE, GLY-GLY, HEPPS, BICINE, TAPS, A MPD, A MPSO, CHES, CAPSO, AMP, CAPS, IB1, TCEP, EGTA, Tween20, KCl, KOH, MgCl2, glycerol, or any combination thereof. In some instances, a buffer may comprise Tris-HCl pH 8.8, VLB, EGTA, CH₃COOH, TCEP, IsoAmpR, (NH₄)₂SO₄, KCl, MgSO₄, Tween20, KOAc, MgOAc, BSA, phosphate, citrate, acetate, imidazole, or any combination thereof. In some embodiments, the concentration of the buffering agent in the buffer is 1 mM to 200 mM. A buffer compatible with an effector protein may comprise a buffering agent at a concentration of 10 mM to 30 mM. A buffer compatible with an effector protein may comprise a buffering agent at a concentration of about 20 mM. A buffering agent may provide a pH for the buffer or the solution in which the activity of the effector protein occurs. The pH may be 3 to 4, 3.5 to 4.5, 4 to 5, 4.5 to 5.5, 5 to 6, 5.5 to 6.5, 6 to 7, 6.5 to 7.5, 7 to 8, 7.5 to 8.5, 8 to 9, 8.5 to 9.5, 9 to 10, or 9.5 to 10.5.

In some embodiments, systems comprise a solution, wherein the solution comprises at least one salt. In some embodiments, the at least one salt is selected from magnesium salt, a zinc salt, a potassium salt, a calcium salt, and a sodium. In some embodiments, the salt is a combination of two or more salts. For example, in some embodiments, the salt is a combination of two or more salts selected from a magnesium salt, a zinc salt, a potassium salt, a calcium salt and a sodium salt. In some embodiments, the at least one salt is selected from potassium acetate, magnesium acetate, sodium chloride, potassium chloride, magnesium chloride, calcium chloride, and any combination thereof.

In some embodiments, the concentration of the one or more salt in the solution is about 0.001 mM to about 500 mM. In some embodiments, the concentration of the salt is about 0.001 mM to about 400 mM. In some embodiments, the concentration of the salt is about 0.001 mM to about 300 mM. In some embodiments, the concentration of the salt is about 0.001 mM to about 200 mM. In some embodiments, the concentration of the salt is about 0.001 mM to about 100 mM. In some embodiments, the concentration of the salt is about 0.001 mM to about 10 mM. In some embodiments, the concentration of the salt is about 0.01 mM to about 500 mM. In some embodiments, the concentration of the salt is about 0.01 mM to about 400 mM. In some embodiments, the concentration of the salt is about 0.01 mM to about 300 mM. In some embodiments, the concentration of the salt is about 0.01 mM to about 200 mM. In some embodiments, the concentration of the salt is about 0.01 mM to about 100 mM. In some embodiments, the concentration of the salt is about 0.01 mM to about 10 mM. In some embodiments, the concentration of the salt is about 0.1 mM to about 500 mM. In some embodiments, the concentration of the salt is about 0.1 mM to about 400 mM. In some embodiments, the concentration of the salt is about 0.1 mM to about 300 mM. In some embodiments, the concentration of the salt is about 0.1 mM to about 200 mM. In some embodiments, the concentration of the salt is about 0.1 mM to about 100 mM. In some embodiments, the concentration of the salt is about 0.1 mM to about 10 mM. In some embodiments, the concentration of the salt is about 1 mM to about 500 mM. In some embodiments, the concentration of the salt is about 1 mM to about 400 mM. In some embodiments, the concentration of the salt is about 1 mM to about 300 mM. In some embodiments, the concentration of the salt is about 1 mM to about 200 mM. In some embodiments, the concentration of the salt is about 1 mM to about 100 mM. In some embodiments, the concentration of the salt is about 1 mM to about 10 mM. In some embodiments, the concentration of the salt is about 10 mM to about 500 mM. In some embodiments, the concentration of the salt is about 10 mM to about 400 mM. In some embodiments, the concentration of the salt is about 10 mM to about 300 mM. In some embodiments, the concentration of the salt is about 10 mM to about 200 mM. In some embodiments, the concentration of the salt is about 10 mM to about 100 mM. In some embodiments, the concentration of the salt is about 100 mM to about 500 mM. In some embodiments, the concentration of the salt is about 100 mM to about 400 mM. In some embodiments, the concentration of the salt is about 100 mM to about 300 mM. In some embodiments, the concentration of the salt is about 100 mM to about 200 mM. In some embodiments, the concentration of the at least one salt in the solution is 5 mM to 100 mM, 5 mM to 10 mM, 1 mM to 60 mM, or 1 mM to 10 mM. In some embodiments, the concentration of the at least one salt is about 105 mM. In some embodiments, the concentration of the at least one salt is about 55 mM. In some embodiments, the concentration of the at least one salt is about 7 mM.

In some embodiments, the solution comprises potassium acetate and magnesium acetate. In some embodiments, the solution comprises sodium chloride and magnesium chloride. In some embodiments, the solution comprises potassium chloride and magnesium chloride. In some embodiments, the salt is a magnesium salt and the concentration of magnesium in the solution is at least 5 mM, 7 mM, at least 9 mM, at least 11 mM, at least 13 mM, or at least 15 mM. In some embodiments, the concentration of magnesium is less than 20 mM, less than 18 mM, or less than 16 mM.

In some embodiments, systems comprise a solution, wherein the solution comprises at least one crowding agent. A crowding agent may reduce the volume of solvent available for other molecules in the solution, thereby increasing the effective concentrations of said molecules. Exemplary crowding agents include glycerol and bovine serum albumin. In some embodiments, the crowding agent is glycerol. In some embodiments, the concentration of the crowding agent in the solution is 0.01% (v/v) to 10% (v/v). In some embodiments, the concentration of the crowding agent in the solution is 0.5% (v/v) to 10% (v/v).

In some embodiments, systems comprise a solution, wherein the solution comprises at least one detergent. Exemplary detergents include Tween. Triton-X, and IGEPAL. A solution may comprise Tween. Triton-X, or any combination thereof. A solution may comprise Triton-X. A solution may comprise IGEPAL CA-630. In some embodiments, the concentration of the detergent in the solution is 2% (v/v) or less. In some embodiments, the concentration of the detergent in the solution is 1% (v/v) or less. In some embodiments, the concentration of the detergent in the solution is 0.00001% (v/v) to 0.01% (v/v). In some embodiments, the concentration of the detergent in the solution is about 0.01% (v/v).

In some embodiments, systems comprise a solution, wherein the solution comprises at least one reducing agent. Exemplary reducing agents comprise dithiothreitol (DTT). β-mercaptoethanol (BME), or tris(2-carboxyethyl) phosphine (TCEP). In some embodiments, the reducing agent is DTT. In some embodiments, the concentration of the reducing agent in the solution is 0.01 mM to 100 mM. In some embodiments, the concentration of the reducing agent in the solution is 0.1 mM to 10 mM. In some embodiments, the concentration of the reducing agent in the solution is 0.5 mM to 2 mM. In some embodiments, the concentration of the reducing agent in the solution is 0.01 mM to 100 mM. In some embodiments, the concentration of the reducing agent in the solution is 0.1 mM to 10 mM. In some embodiments, the concentration of the reducing agent in the solution is about 1 mM.

In some embodiments, systems comprise a solution, wherein the solution comprises a competitor. In general, competitors compete with the target nucleic acid or the reporter nucleic acid for cleavage by the effector protein or a dimer thereof. Exemplary competitors include heparin, and imidazole, and salmon sperm DNA. In some embodiments, the concentration of the competitor in the solution is 1 μg/mL to 100 μg/mL. In some embodiments, the concentration of the competitor in the solution is 40 μg/mL to 60 μg/mL.

In some embodiments, systems comprise a solution, wherein the solution comprises a co-factor. In some embodiments, the co-factor allows an effector protein or a multimeric complex thereof to perform a function, including pre-crRNA processing and/or target nucleic acid cleavage. The suitability of a cofactor for an effector protein or a multimeric complex thereof may be assessed, such as by methods based on those described by Sundaresan et al. (Cell Rep. 2017 Dec. 26; 21 (13): 3728-3739). In some embodiments, an effector or a multimeric complex thereof forms a complex with a co-factor. In some embodiments, the co-factor is a divalent metal ion. In some embodiments, the divalent metal ion is selected from Mg²⁺, Mn²⁺, Zn²⁺. Ca²⁺, Cu²⁺. In some embodiments, the divalent metal ion is Mg²⁺. In some embodiments, the co-factor is Mg²⁺.

In some embodiments, systems, and compositions for use with systems comprise a catalytic reagent for signal improvement or enhancement. In some embodiments, the catalytic reagent enhances signal generation via hydrolysis of inorganic pyrophosphates. In some embodiments, catalytic reagents enhance signal generation via enhancement of DNA replication. In some embodiments, catalytic reagents enhance signal amplification via revival of ions (e.g., Mg²⁺) in a buffer, thereby enhancing the function of an effector protein. In some embodiments, the catalytic reagent for signal improvement may be an enzyme. In some embodiments, the catalytic reagent for signal improvement are particularly useful in amplification and/or detection reactions as described herein. Other exemplary reagents useful for amplification and/or detection reactions (i.e., amplification and detection reagents, respectively) are described throughout herein.

Any of the systems, methods, or compositions described herein may comprise a catalytic reagent or the use thereof. In some embodiments, compositions comprise about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, or 10 enzyme unit (U) of a catalytic reagent per 10 μL of solution. In some embodiments, a catalytic reagent is present in a composition at a concentration of 0.125 Units, 0.5 Units, 0.25 Units, 1.0 Units, 2.0 Units, 2.5 Units, or 4 Units per discrete reaction volume. In some embodiments, a catalytic reagent is provided in a system separately from a buffer provided in the system. In some embodiments, systems comprise a buffer, wherein a catalytic reagent is provided in the buffer.

In some embodiments, a catalytic reagent improves the signal to noise ratio of an effector protein-based detection reaction. In some embodiments, a catalytic reagent improves overall signal (e.g., fluorescence of a cleaved reporter). A catalytic reagent may improve signal by a factor, wherein the signal is indicative of the presence of a target nucleic acid. In some embodiments, the factor may be at least about 1.1, at least about 2, at least about 3, at least about 4, at least about 5, at least about 6, at least about 7, at least about 8, at least about 9, or at least about 10.

Also provided herein are reagents for: detection reactions, nuclease purification, cell lysis, in vitro transcription reactions, amplification reactions, reverse transcription reactions, and the like. In some embodiments, systems, compositions, and/or solutions described herein comprise one or more of: detection reagents, nuclease purification reagents, cell lysis reagents, in vitro transcription reagents, amplification reagents, reverse transcription reagents, and combinations thereof. In some embodiments, any such reagents suitable with the compositions, methods, systems, devices, and/or kits described herein may be used to achieve one or more of the foregoing described reactions. Reagents provided herein may be used with any other solution components described herein, including buffers, amino acids or derivatives thereof, chaotrpes, chelators, cyclodextrins, inhibitors, ionic liquids, linkers, metals, non-detergent sulfobetaines, organic acids, osmolytes, peptides, polyamides, polymers, polyols, polyols and salts, salts, or combinations thereof.

### Detection Reagents/Components and Reporters

In some embodiments, systems disclosed herein comprise detection reagents to facilitate detection of nucleic acids as described herein. Non-limiting examples of detection reagents include a reporter nucleic acid, a detection moiety, and additional polypeptides. In some embodiments, the detection reagent is operably linked to an effector protein described herein such that a detection event occurs upon contacting the detection reagent and effector protein with a target nucleic acid. In some embodiments, when describing a detection event, such as in reference to a microfluidic device, reference is generally made to a moment in which compositions within the detection region of a microfluidic device exhibit binding of a programmable nuclease to a guide nucleic acid, binding of a guide nucleic acid to a target nucleic acid or target amplicon, and/or access to and cleavage of a reporter by an activated programmable nuclease, in accordance to the assay(s) being performed. A detection event may produce a detectable product or a detectable signal. In some embodiments, when describing a detectable product, reference is made to a unit produced after the cleavage of a reporter that is capable of being discovered, identified, perceived or noticed. A detectable product can comprise a detectable label and/or moiety that emits a detectable signal. A detectable product may include other components that are not capable of being readily discovered, identified, perceived or noticed at the same time as the detectable signal. For example, a detectable product may comprise remnants of the reporter. Accordingly, in some instances, the detectable product comprises RNA and/or DNA.

Upon the occurrence of the detection event, a signal (e.g., a detectable signal or detectable product) can be generated thereby indicating detection of the target nucleic acid. Any suitable detection reagent may be used, including: a nucleic acid (which may be referred to herein as a detection or reporter nucleic acid), a detection moiety, an additional polypeptide, or a combination thereof. Other detection reagents include buffers, reverse transcriptase mix, a catalytic reagent, a stain, and the like. Any reagents suitable with the detection reactions, events, and signals described herein are useful as detection reagents for the systems, compositions, methods, kits, devices, and solutions provided herein. In some embodiments, detection reagents are capable of detecting a nucleic acid in a sample.

In some embodiments, systems, compositions, methods, kits, devices, and solutions comprise 0.01 μL, 0.02 μL, 0.03 μL, 0.04 μL, 0.05 μL, 0.06 μL, 0.07 μL, 0.08 μL, 0.09 μL, 0.1 μL, 0.2 μL, 0.3 μL, 0.4 μL, 0.5 μL, 0.6 μL, 0.7 μL, 0.8 μL, 0.9 μL, 1 μL, 2 μL, 3 μL, 4 μL, 5 μL, 6 μL, 7 L, 8 μL, 9 μL, 10 μL, 20 μL, 30 μL, 40 μL, 50 μL, 60 μL, 70 μL, 80 μL, 90 μL, 100 μL, 150 μL, 200 μL, 250 μL, 300 μL, 350 μL, 400 μL, 450 μL, 500 μL, or more of each detection reagent as described herein. In some embodiments, systems, compositions, methods, kits, devices, and solutions comprise 1 nM, 2 nM, 3 nM, 4 nM, 5 nM, 6 nM, 7 nM, 8 nM, 9 nM, 10 nM, 20 nM, 30 nM, 40 nM, 50 nM, 60 nM, 70 nM, 80 nM, 90 nM, 100 nM, 150 nM, 200 nM, 250 nM, 300 nM, 350 nM, 400 nM, 450 nM, 500 nM, or more of each detection reagent as described herein. In some embodiments, systems, compositions, methods, kits, devices, and solutions comprise 1 μM, 2 μM, 3 μM, 4 μM, 5 μM, 6 μM, 7 μM, 8 μM, 9 μM, 10 μM, 20 μM, 30 μM, 40 μM, 50 μM, 60 μM, 70 μM, 80 μM, 90 μM, 100 μM, 150 μM, 200 μM, 250 μM, 300 μM, 350 μM, 400 μM, 450 μM, 500 μM, or more of each detection reagent as described herein. In some embodiments, systems, compositions, methods, kits, devices, and solutions comprise 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, 150 mM, 200 mM, 250 mM, 300 mM, 350 mM, 400 mM, 450 mM, 500 mM, or more of each detection reagent as described herein.

In some embodiments, detection reagents are capable of detecting a nucleic acid in a sample. Nucleic acid amplification of the target nucleic acid may improve at least one of sensitivity, specificity, or accuracy of the assay in detecting the target nucleic acid. Accordingly, in some embodiments, nucleic acid detection involves PCR or isothermal nucleic acid amplification, providing improved sensitive, specific, or rapid detection. The reagents or components for nucleic acid detection may comprise recombinases, primers, polypeptides, buffers, and signal reagents suitable for a detection reaction.

In some embodiments, systems described herein comprise a PCR tube, a PCR well or a PCR plate. In some embodiments, the wells of the PCR plate may be pre-aliquoted with the reagent for detecting a nucleic acid, as well as a guide nucleic acid, an effector protein, a multimeric complex, an amplification reagent, or any combination thereof. In some embodiments, the pre-aliquoted guide nucleic acid targeting a target sequence, and an effector protein capable of being activated when complexed with the guide nucleic acid and the target sequence. A user may thus add a sample of interest to a well of the pre-aliquoted PCR plate.

In some embodiments, nucleic acid detection is performed in a nucleic acid detection region on a support medium, or sample interface. In some embodiments, when describing a detection region, such as in reference to a microfluidic device, reference is generally made to a structural component which may comprise detection reagents that are immobilized, dried, or otherwise deposited thereto, including guide nucleic acids and/or reporters. A detection region may comprise one or more dried and/or immobilized amplification reagents including primers, polymerases, reverse transcriptase, and/or dNTPs. In some instances, a detection region may comprise a single detection array, one or more lateral flow strips, a detection tray, a capture antibody, or combinations thereof. Accordingly, in some instances, a detection region may comprise a plurality of microwells, detection chambers or channels, in fluid communication with amplification region(s). By way of a non-limiting example, a detection region may comprise three parallel detection chambers, each coupled to a single amplification region. One of ordinary skill in the art will recognize that the relative numbers of and relationships between amplification region(s) and detection region(s) may be varied depending on the assay(s) being performed. Also by way of a non-limiting example, compositions within the detection region of a microfluidic device may be agitated (e.g., via a spring-loaded valve piston) to facilitate binding of a effector protein to a guide nucleic acid, binding of a guide nucleic acid to a target nucleic acid or target amplicon, and/or access to and cleavage of a reporter by an activated effector protein.

Alternatively, or in combination, the nucleic acid detection is performed in a reagent chamber, and the resulting sample is applied to the support medium, sample interface, or surface within a reagent chamber.

In some embodiments, the reporter nucleic acid is capable of being cleaved by the activated nuclease, thereby generating a detectable signal. A user may thus add a sample of interest to a well of the pre-aliquoted PCR plate and measure for the detectable signal with a fluorescent light reader or a visible light reader.

In some embodiments, detection reaction of nucleic acid as described herein is performed for no greater than 1, 2, 3, 4, 5, 6, 7, 8, 9, 10, 11, 12, 13, 14, 15, 16, 17, 18, 19, 20, 25, 30, 40, 50, or 60 minutes, or any value 1 to 60 minutes. In some embodiments, the detection reaction is performed for 1 to 60, 5 to 55, 10 to 50, 15 to 45, 20 to 40, or 25 to 35 minutes. In some embodiments, the detection reaction is performed at a temperature of around 20-45° C. In some embodiments, the detection reaction is performed at a temperature no greater than 20° C., 25° C., 30° C., 35° C., 37° C., 40° C., 45° C., or any value 20° C., to 45° C. In some embodiments, the detection reaction is performed at a temperature of at least 20° C., 25° C., 30° C., 35° C., 37° C., 40° C., or 45° C., or any value 20° C., to 45° C. In some embodiments, the detection reaction is performed at a temperature of 20° C., to 45° C., 25° C., to 40° C., 30° C., to 40° C., or 35° C., to 40° C.

In some embodiments, the reagents or components for detecting a nucleic acid are, for example, consistent for use with various devices disclosed herein for detection of a target nucleic acid within the sample, wherein a device may comprise multiple pumps, valves, reservoirs, and chambers for sample preparation, amplification of a target nucleic acid within the sample, mixing with an effector protein, and detection of a detectable signal arising from cleavage of detector nucleic acids by an effector protein within a system itself. These reagents are compatible with the samples, devices, methods of detection, and support mediums as described herein for detection of an ailment, such as a disease, or genetic disorder, or genetic information, such as for phenotyping, genotyping, or determining ancestry. The reagents described herein for detecting a disease, or genetic disorder comprise a guide nucleic acid targeting the target nucleic acid segment indicative of a disease, or genetic disorder.

In some embodiments, systems disclosed herein comprise a reporter. By way of non-limiting and illustrative example, a reporter may comprise a single stranded nucleic acid and a detection moiety (e.g., a labeled single stranded RNA reporter), wherein the nucleic acid is capable of being cleaved by an effector protein (e.g., a CRISPR/Cas protein as disclosed herein) or a multimeric complex thereof, releasing the detection moiety, and generating a detectable signal or detectable product. As used herein. “reporter” is used interchangeably with “reporter nucleic acid” or “reporter molecule”. The effector proteins disclosed herein, activated upon hybridization of a guide nucleic acid to a target nucleic acid, may cleave the reporter. Cleaving the “reporter” may be referred to herein as cleaving the “reporter nucleic acid.” the “reporter molecule.” or the “nucleic acid of the reporter.” Cleavage of a reporter may produce different types of signals (e.g., a detectable signal). In some cases, cleavage of the reporter can produce a calorimetric signal, a potentiometric signal, an amperometric signal, an optical signal, or a piezo-electric signal. Various devices and/or sensors can be used to detect these different types of signals, which indicate whether a target nucleic acid, is present in the sample. The sensors usable to detect such signals can include, for example, optical sensors (e.g., imaging devices for detecting fluorescence or optical signals with various wavelengths and frequencies), electric potential sensors, surface plasmon resonance (SPR) sensors, interferometric sensors, or any other type of sensor suitable for detecting calorimetric signals, potentiometric signals, amperometric signals, optical signals, or piezo-electric signals.

Reporters may comprise RNA. Reporters may comprise DNA. Reporters may be double-stranded. Reporters may be single-stranded. In some embodiments, reporters comprise a protein capable of generating a signal. In some embodiments, a reporter may comprise a protein capable of generating a detectable signal or signal. In some embodiments, a reporter may be operably linked to the protein capable of generating a signal. A signal may be a calorimetric, potentiometric, amperometric, optical (e.g., fluorescent, colorimetric, etc.), or piezo-electric signal. In some embodiments, the reporter comprises a detection moiety. In some embodiments, the reporter comprises a detection moiety. In some embodiments, the reporter is configured to release a detection moiety or generate a signal indicative of a presence or absence of the target nucleic acid. For example, the signal can indicate a presence of the target nucleic acid in the sample, and an absence of the signal can indicate an absence of the target nucleic acid in the sample. Suitable detectable labels and/or moieties that may provide a signal include, but are not limited to, an enzyme, a radioisotope, a member of a specific binding pair; a fluorophore; a fluorescent protein; a quantum dot; and the like.

In some embodiments, the reporter comprises a detection moiety and a quenching moiety. In some embodiments, the reporter comprises a cleavage site, wherein the detection moiety is located at a first site on the reporter and the quenching moiety is located at a second site on the reporter, wherein the first site and the second site are separated by the cleavage site. Sometimes the quenching moiety is a fluorescence quenching moiety. In some embodiments, the quenching moiety is 5′ to the cleavage site and the detection moiety is 3′ to the cleavage site. In some embodiments, the detection moiety is 5′ to the cleavage site and the quenching moiety is 3′ to the cleavage site. Sometimes the quenching moiety is at the 5′ terminus of the nucleic acid of a reporter. Sometimes the detection moiety is at the 3″ terminus of the nucleic acid of a reporter. In some embodiments, the detection moiety is at the 5′ terminus of the nucleic acid of a reporter. In some embodiments, the quenching moiety is at the 3″ terminus of the nucleic acid of a reporter.

Suitable fluorescent proteins include, but are not limited to, green fluorescent protein (GFP) or variants thereof, blue fluorescent variant of GFP (BFP), cyan fluorescent variant of GFP (CFP), yellow fluorescent variant of GFP (YFP), enhanced GFP (EGFP), enhanced CFP (ECFP), enhanced YFP (EYFP), GFPS65T, Emerald, Topaz (TYFP), Venus, Citrine, mCitrine, GFPuv, destabilised EGFP (dEGFP), destabilised ECFP (dECFP), destabilised EYFP (dEYFP), mCFPm, Cerulean, T-Sapphire, CyPet, YPet, mKO, HcRed, t-HcRed, DsRed, DsRed2, DsRed-monomer, J-Red, dimer2, t-dimer2 (12), mRFP1, pocilloporin, Renilla GFP, Monster GFP, paGFP, Kaede protein and kindling protein, Phycobiliproteins and Phycobiliprotein conjugates including B-Phycoerythrin, R-Phycoerythrin and Allophycocyanin. Suitable enzymes include, but are not limited to, horseradish peroxidase (HRP), alkaline phosphatase (AP), beta-galactosidase (GAL), glucose-6-phosphate dehydrogenase, beta-N-acetylglucosaminidase. β-glucuronidase, invertase. Xanthine Oxidase, firefly luciferase, and glucose oxidase (GO).

In some embodiments, the detection moiety comprises an invertase. The substrate of the invertase may be sucrose. A DNS reagent may be included in the system to produce a colorimetric change when the invertase converts sucrose to glucose. In some embodiments, the reporter nucleic acid and invertase are conjugated using a heterobifunctional linker via sulfo-SMCC chemistry.

Suitable fluorophores may provide a detectable fluorescence signal in the same range as 6-Fluorescein (Integrated DNA Technologies). IRDye 700 (Integrated DNA Technologies). TYE 665 (Integrated DNA Technologies). Alex Fluor 594 (Integrated DNA Technologies), or ATTO™ 633 (NHS Ester) (Integrated DNA Technologies). Non-limiting examples of fluorophores are fluorescein amidite, 6-Fluorescein, IRDye 700. TYE 665. Alex Fluor 594, or ATTO™ 633 (NHS Ester). The fluorophore may be an infrared fluorophore. The fluorophore may emit fluorescence in the range of 500 nm and 720 nm. In some embodiments, the fluorophore emits fluorescence at a wavelength of 700 nm or higher. In other cases, the fluorophore emits fluorescence at about 665 nm. In some embodiments, the fluorophore emits fluorescence in the range of 500 nm to 520 nm, 500 nm to 540 nm, 500 nm to 590 nm, 590 nm to 600 nm, 600 nm to 610 nm, 610 nm to 620 nm, 620 nm to 630 nm, 630 nm to 640 nm, 640 nm to 650 nm, 650 nm to 660 nm, 660 nm to 670 nm, 670 nm to 680 nm, 690 nm to 690 nm, 690 nm to 700 nm, 700 nm to 710 nm, 710 nm to 720 nm, or 720 nm to 730 nm. In some embodiments, the fluorophore emits fluorescence in the range 450 nm to 750 nm, 500 nm to 650 nm, or 550 to 650 nm.

Systems may comprise a quenching moiety. A quenching moiety may be chosen based on its ability to quench the detection moiety. A quenching moiety may be a non-fluorescent fluorescence quencher. A quenching moiety may quench a detection moiety that emits fluorescence in the range of 500 nm and 720 nm. A quenching moiety may quench a detection moiety that emits fluorescence in the range of 500 nm and 720 nm. In some embodiments, the quenching moiety quenches a detection moiety that emits fluorescence at a wavelength of 700 nm or higher. In other cases, the quenching moiety quenches a detection moiety that emits fluorescence at about 660 nm or about 670 nm. In some embodiments, the quenching moiety quenches a detection moiety that emits fluorescence in the range of 500 to 520, 500 to 540, 500 to 590, 590 to 600, 600 to 610, 610 to 620, 620 to 630, 630 to 640, 640 to 650, 650 to 660, 660 to 670, 670 to 680, 690 to 690, 690 to 700, 700 to 710, 710 to 720, or 720 to 730 nm. In some embodiments, the quenching moiety quenches a detection moiety that emits fluorescence in the range 450 nm to 750 nm, 500 nm to 650 nm, or 550 to 650 nm. A quenching moiety may quench fluorescein amidite, 6-Fluorescein. IRDye 700. TYE 665. Alex Fluor 594, or ATTO™ 633 (NHS Ester). A quenching moiety may be lowa Black RQ. Iowa Black FQ or IRDye QC-1 Quencher. A quenching moiety may quench fluorescein amidite, 6-Fluorescein (Integrated DNA Technologies). IRDye 700 (Integrated DNA Technologies). TYE 665 (Integrated DNA Technologies). Alex Fluor 594 (Integrated DNA Technologies), or ATTO™ 633 (NHS Ester) (Integrated DNA Technologies). A quenching moiety may be lowa Black RQ (Integrated DNA Technologies), Iowa Black FQ (Integrated DNA Technologies) or IRDye QC-1 Quencher (LiCor). Any of the quenching moieties described herein may be from any commercially available source, may be an alternative with a similar function, a generic, or a non-tradename of the quenching moieties listed.

The generation of the detectable product or detectable signal from the release of the detection moiety may indicate that cleavage by the effector protein has occurred and that the sample contains the target nucleic acid. In some embodiments, the detection moiety comprises a fluorescent dye. Sometimes the detection moiety comprises a fluorescence resonance energy transfer (FRET) pair. In some embodiments, the detection moiety comprises an infrared (IR) dye. In some embodiments, the detection moiety comprises an ultraviolet (UV) dye. Alternatively, or in combination, the detection moiety comprises a protein. Sometimes the detection moiety comprises a biotin. Sometimes the detection moiety comprises an antigen. Sometimes the detection moiety comprises at least one of avidin or streptavidin. In some embodiments, the detection moiety comprises a polysaccharide, a polymer, or a nanoparticle. In some embodiments, the detection moiety comprises a gold nanoparticle or a latex nanoparticle.

A detection moiety may be any moiety capable of generating a detectable product or detectable signal upon cleavage of the reporter by the effector protein. The detectable product may be a detectable unit generated from the detectable moiety and capable of emitting a detectable signal as described herein. In some embodiments, the detectable product further comprises a detectable label, a fluorophore, a reporter, or a combination thereof. In some embodiments, the detectable product comprises RNA, DNA, or both. In some embodiments, the detectable product is configured to generate a signal indicative of the presence or absence of the target nucleic acid in, for instance, a cell or a sample.

A detection moiety may be any moiety capable of generating a calorimetric, potentiometric, amperometric, optical (e.g., fluorescent, colorimetric, etc.), or piezo-electric signal. A nucleic acid of a reporter, sometimes, is protein-nucleic acid that is capable of generating a calorimetric, potentiometric, amperometric, optical (e.g., fluorescent, colorimetric, etc.), or piezo-electric signal upon cleavage of the nucleic acid. Often a calorimetric signal is heat produced after cleavage of the nucleic acids of a reporter. Sometimes, a calorimetric signal is heat absorbed after cleavage of the nucleic acids of a reporter. A potentiometric signal, for example, is electrical potential produced after cleavage of the nucleic acids of a reporter. An amperometric signal may be movement of electrons produced after the cleavage of nucleic acid of a reporter. Often, the signal is an optical signal, such as a colorimetric signal or a fluorescence signal. An optical signal is, for example, a light output produced after the cleavage of the nucleic acids of a reporter. Sometimes, an optical signal is a change in light absorbance between before and after the cleavage of nucleic acids of a reporter. Often, a piezo-electric signal is a change in mass between before and after the cleavage of the nucleic acid of a reporter.

The detectable signal may be a colorimetric signal or a signal visible by eye. In some embodiments, the detectable signal may be fluorescent, electrical, chemical, electrochemical, or magnetic. In some embodiments, the first detection signal may be generated by binding or interaction of the detection moiety to the capture molecule in the detection region, where the first detection signal indicates that the sample contained the target nucleic acid. Sometimes systems are capable of detecting more than one type of target nucleic acid, wherein the system comprises more than one type of guide nucleic acid and more than one type of reporter nucleic acid. In some embodiments, the detectable signal may be generated directly by the cleavage event. Alternatively, or in combination, the detectable signal may be generated indirectly by the signal event. Sometimes the detectable signal is not a fluorescent signal. In some embodiments, the detectable signal may be a colorimetric or color-based signal. In some embodiments, the detected target nucleic acid may be identified based on its spatial location on the detection region of the support medium. In some embodiments, the second detectable signal may be generated in a spatially distinct location than the first generated signal.

In some embodiments, the reporter nucleic acid is a single-stranded nucleic acid sequence comprising ribonucleotides. The nucleic acid of a reporter may be a single-stranded nucleic acid sequence comprising at least one ribonucleotide. In some embodiments, the nucleic acid of a reporter is a single-stranded nucleic acid comprising at least one ribonucleotide residue at an internal position that functions as a cleavage site. In some embodiments, the nucleic acid of a reporter comprises at least 2, at least 3, at least 4, at least 5, at least 6, at least 7, at least 8, at least 9, or at least 10 ribonucleotide residues at an internal position. In some embodiments, the nucleic acid of a reporter comprises from 2 to 10, from 3 to 9, from 4 to 8, or from 5 to 7 ribonucleotide residues at an internal position. Sometimes the ribonucleotide residues are continuous. Alternatively, the ribonucleotide residues are interspersed in between non-ribonucleotide residues. In some embodiments, the nucleic acid of a reporter has only ribonucleotide residues. In some embodiments, the nucleic acid of a reporter has only deoxyribonucleotide residues. In some embodiments, the nucleic acid comprises nucleotides resistant to cleavage by the effector protein described herein. In some embodiments, the nucleic acid of a reporter comprises synthetic nucleotides. In some embodiments, the nucleic acid of a reporter comprises at least one ribonucleotide residue and at least one non-ribonucleotide residue.

In some embodiments, the nucleic acid of a reporter comprises at least one uracil ribonucleotide. In some embodiments, the nucleic acid of a reporter comprises at least two uracil ribonucleotides. Sometimes the nucleic acid of a reporter has only uracil ribonucleotides. In some embodiments, the nucleic acid of a reporter comprises at least one adenine ribonucleotide. In some embodiments, the nucleic acid of a reporter comprises at least two adenine ribonucleotides. In some embodiments, the nucleic acid of a reporter has only adenine ribonucleotides. In some embodiments, the nucleic acid of a reporter comprises at least one cytosine ribonucleotide. In some embodiments, the nucleic acid of a reporter comprises at least two cytosine ribonucleotides. In some embodiments, the nucleic acid of a reporter comprises at least one guanine ribonucleotide. In some embodiments, the nucleic acid of a reporter comprises at least two guanine ribonucleotides. In some embodiments, a nucleic acid of a reporter comprises a single unmodified ribonucleotide. In some embodiments, a nucleic acid of a reporter comprises only unmodified deoxyribonucleotides.

In some embodiments, the nucleic acid of a reporter is 5 to 20, 5 to 15, 5 to 10, 7 to 20, 7 to 15, or 7 to 10 nucleotides in length. In some embodiments, the nucleic acid of a reporter is 3 to 20, 4 to 10, 5 to 10, or 5 to 8 nucleotides in length. In some embodiments, the nucleic acid of a reporter is 5 to 12 nucleotides in length. In some embodiments, the reporter nucleic acid is at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 nucleotides in length. In some embodiments, the reporter nucleic acid is 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length.

In some embodiments, systems comprise a plurality of reporters. The plurality of reporters may comprise a plurality of signals. In some embodiments, systems comprise at least 2, at least 3, at least 4, at least 5, at least 6, at least 7, at least 8, at least 9, at least 10, at least 11, at least 12, at least 13, at least 14, at least 15, at least 20, at least 30, at least 40, or at least 50 reporters. In some embodiments, there are 2 to 50, 3 to 40, 4 to 30, 5 to 20, or 6 to 10 different reporters.

In some embodiments, systems comprise an effector protein and a reporter nucleic acid configured to undergo trans cleavage by the effector protein, trans cleavage of the reporter may generate a signal from the reporter or alter a signal from the reporter. In some embodiments, the signal is an optical signal, such as a fluorescence signal or absorbance band, trans cleavage of the reporter may alter the wavelength, intensity, or polarization of the optical signal. For example, the reporter may comprise a fluorophore and a quencher, such that trans cleavage of the reporter separates the fluorophore and the quencher thereby increasing a fluorescence signal from the fluorophore. Herein, detection of reporter cleavage to determine the presence of a target nucleic acid may be referred to as DETECTR′. In some embodiments described herein is a method of assaying for a target nucleic acid in a sample comprising contacting the target nucleic acid with an effector protein, a non-naturally occurring guide nucleic acid that hybridizes to a segment of the target nucleic acid, and a reporter nucleic acid, and assaying for a change in a signal, wherein the change in the signal is produced by cleavage of the reporter nucleic acid.

In the presence of a large amount of non-target nucleic acids, an activity of an effector protein (e.g., an effector protein as disclosed herein) may be inhibited. This is because the activated effector proteins collaterally cleave any nucleic acids. If total nucleic acids are present in large amounts, they may outcompete reporters for the effector proteins. In some embodiments, systems comprise an excess of reporter(s), such that when the system is operated and a solution of the system comprising the reporter is combined with a sample comprising a target nucleic acid, the concentration of the reporter in the combined solution-sample is greater than the concentration of the target nucleic acid. In some embodiments, the sample comprises amplified target nucleic acid. In some embodiments, the sample comprises an unamplified target nucleic acid. In some embodiments, the concentration of the reporter is greater than the concentration of target nucleic acids and non-target nucleic acids. The non-target nucleic acids may be from the original sample, either lysed or unlysed. The non-target nucleic acids may comprise byproducts of amplification. In some embodiments, systems comprise a reporter wherein the concentration of the reporter in a solution 1.5 fold, at least 2 fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14 fold, at least 15 fold, at least 16 fold, at least 17 fold, at least 18 fold, at least 19 fold, at least 20 fold, at least 30 fold, at least 40 fold, at least 50 fold, at least 60 fold, at least 70 fold, at least 80 fold, at least 90 fold, at least 100 fold excess of total nucleic acids. In some embodiments, systems comprise a reporter wherein the concentration of the reporter in a solution 1.5 fold to 100 fold, 2 fold to 10 fold, 10 fold to 20 fold, 20 fold to 30 fold, 30 fold to 40 fold, 40 fold to 50 fold, 50 fold to 60 fold, 60 fold to 70 fold, 70 fold to 80 fold, 80 fold to 90 fold, 90 fold to 100 fold, 1.5 fold to 10 fold, 1.5 fold to 20 fold, 10 fold to 40 fold, 20 fold to 60 fold, or 10 fold to 80 fold excess of total nucleic acids.

### Amplification Reagents/Components

In some embodiments, systems described herein comprise a reagent or component for amplifying a nucleic acid. Non-limiting examples of reagents for amplifying a nucleic acid include polymerases, primers, and nucleotides. In some embodiments, systems comprise reagents for nucleic acid amplification of a target nucleic acid in a sample. Nucleic acid amplification of the target nucleic acid may improve at least one of sensitivity, specificity, or accuracy of the assay in detecting the target nucleic acid. In some embodiments, nucleic acid amplification is isothermal nucleic acid amplification, providing for the use of the system or system in remote regions or low resource settings without specialized equipment for amplification. In some embodiments, amplification of the target nucleic acid increases the concentration of the target nucleic acid in the sample relative to the concentration of nucleic acids that do not correspond to the target nucleic acid.

The reagents for nucleic acid amplification may comprise a recombinase, an oligonucleotide primer, a single-stranded DNA binding (SSB) protein, a polymerase, or a combination thereof that is suitable for an amplification reaction. In some embodiments, the reagents for nucleic acid amplification may comprise a recombinase, a primer, an oligonucleotide primer, an activator, a deoxynucleoside triphosphate (dNTP), a ribonucleoside tri-phosphate (rNTP), a single-stranded DNA binding (SSB) protein. Rnase inhibitor, water, a polymerase, reverse transcriptase mix, or a combination thereof that is suitable for an amplification reaction. Non-limiting examples of amplification reactions are transcription mediated amplification (TMA), helicase dependent amplification (HDA), or circular helicase dependent amplification (cHDA), strand displacement amplification (SDA), recombinase polymerase amplification (RPA), loop mediated amplification (LAMP), exponential amplification reaction (EXPAR), rolling circle amplification (RCA), ligase chain reaction (LCR), simple method amplifying RNA targets (SMART), single primer isothermal amplification (SPIA), multiple displacement amplification (MDA), nucleic acid sequence based amplification (NASBA), hinge-initiated primer-dependent amplification of nucleic acids (HIP), nicking enzyme amplification reaction (NEAR), and improved multiple displacement amplification (IMDA).

Such amplification reactions may also be used in combination with reverse transcription (RT) of an RNA of interest. Accordingly, also provided herein are reagents for both the reverse transcription and amplification of nucleic acids. In some embodiments, systems, compositions, methods, kits, devices, and solutions comprise 0.01 μL, 0.02 μL, 0.03 μL, 0.04 μL, 0.05 μL, 0.06 μL, 0.07 μL, 0.08 μL, 0.09 μL, 0.1 μL, 0.2 μL, 0.3 μL, 0.4 μL, 0.5 μL, 0.6 μL, 0.7 μL, 0.8 μL, 0.9 μL, 1 μL, 2 μL, 3 μL, 4 μL, 5 μL, 6 μL, 7 μL, 8 μL, 9 μL, 10 μL, 20 μL, 30 μL, 40 μL, 50 μL, 60 μL, 70 μL, 80 μL, 90 μL, 100 μL, 150 μL, 200 μL, 250 μL, 300 μL, 350 μL, 400 μL, 450 μL, 500 μL, or more of each amplification described herein. In some embodiments, systems, compositions, methods, kits, devices, and solutions comprise 1 nM, 2 nM, 3 nM, 4 nM, 5 nM, 6 nM, 7 nM, 8 nM, 9 nM, 10 nM, 20 nM, 30 nM, 40 nM, 50 nM, 60 nM, 70 nM, 80 nM, 90 nM, 100 nM, 150 nM, 200 nM, 250 nM, 300 nM, 350 nM, 400 nM, 450 nM, 500 nM, or more of each amplification reagent as described herein. In some embodiments, systems, compositions, methods, kits, devices, and solutions comprise 1 μM, 2 μM, 3 HM, 4 μM, 5 μM, 6 μM, 7 μM, 8 μM, 9 μM, 10 μM, 20 μM, 30 μM, 40 μM, 50 μM, 60 μM, 70 μM, 80 μM, 90 μM, 100 μM, 150 μM, 200 μM, 250 μM, 300 μM, 350 μM, 400 μM, 450 μM, 500 μM, or more of each amplification reagent as described herein. In some embodiments, systems, compositions, methods, kits, devices, and solutions comprise 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, 150 mM, 200 mM, 250 mM, 300 mM, 350 mM, 400 mM, 450 mM, 500 mM, or more of each amplification reagent as described herein.

In some embodiments, systems comprise a PCR tube, a PCR well or a PCR plate. The wells of the PCR plate may be pre-aliquoted with the reagent for amplifying a nucleic acid, as well as a guide nucleic acid, an effector protein, a multimeric complex, or any combination thereof. The wells of the PCR plate may be pre-aliquoted with a guide nucleic acid targeting a target sequence, an effector protein capable of being activated when complexed with the guide nucleic acid and the target sequence, and at least one population of a single stranded reporter nucleic acid comprising a detection moiety. In some embodiments, the wells of the PCR plate may be pre-aliquoted with a guide nucleic acid targeting a target sequence, an effector protein capable of being activated when complexed with the guide nucleic acid and the target sequence, an effector protein capable of being activated when complexed with the guide nucleic acid and the target sequence, and at least one population of a single stranded reporter nucleic acid comprising a detection moiety. A user may thus add the biological sample of interest to a well of the pre-aliquoted PCR plate and measure for the detectable signal with a fluorescent light reader or a visible light reader.

In some embodiments, systems comprise a PCR plate; a guide nucleic acid targeting a target sequence: an effector protein capable of being activated when complexed with the guide nucleic acid and the target sequence; and a single stranded reporter nucleic acid comprising a detection moiety, wherein the reporter nucleic acid is capable of being cleaved by the activated nuclease, thereby generating a detectable signal.

In some embodiments, systems comprise a support medium; a guide nucleic acid targeting a target sequence; and an effector protein capable of being activated when complexed with the guide nucleic acid and the target sequence. In some embodiments, nucleic acid amplification is performed in a nucleic acid amplification region on the support medium. Alternatively, or in combination, the nucleic acid amplification is performed in a reagent chamber, and the resulting sample is applied to the support medium.

In some embodiments, a system for modifying a target nucleic acid comprises a PCR plate; a guide nucleic acid targeting a target sequence; and an effector protein capable of being activated when complexed with the guide nucleic acid and the target sequence. The wells of the PCR plate may be pre-aliquoted with the guide nucleic acid targeting a target sequence, and an effector protein capable of being activated when complexed with the guide nucleic acid and the target sequence. A user may thus add the biological sample of interest to a well of the pre-aliquoted PCR plate.

Often, the nucleic acid amplification is performed for no greater than 1, 2, 3, 4, 5, 6, 7, 8, 9, 10, 11, 12, 13, 14, 15, 16, 17, 18, 19, 20, 25, 30, 40, 50, or 60 minutes, or any value 1 to 60 minutes. Sometimes, the nucleic acid amplification is performed for 1 to 60, 5 to 55, 10 to 50, 15 to 45, 20 to 40, or 25 to 35 minutes. Sometimes, the nucleic acid amplification reaction is performed at a temperature of around 20-45° C. In some embodiments, the nucleic acid amplification reaction is performed at a temperature no greater than 20° C., 25° C., 30° C., 35° C., 37° C., 40° C., 45° C., or any value 20° C., to 45° C. In some embodiments, the nucleic acid amplification reaction is performed at a temperature of at least 20° C., 25° C., 30° C., 35° C., 37° C., 40° C., or 45° C., or any value 20° C., to 45° C. In some embodiments, the nucleic acid amplification reaction is performed at a temperature of 20° C., to 45° C., 25° C., to 40° C., 30° C., to 40° C., or 35° C., to 40° C.

Often, systems comprise primers for amplifying a target nucleic acid to produce an amplification product comprising the target nucleic acid and a PAM. For instance, at least one of the primers may comprise the PAM that is incorporated into the amplification product during amplification. The compositions for amplification of target nucleic acids and methods of use thereof, as described herein, are compatible with any of the methods disclosed herein including methods of assaying for at least one base difference (e.g., assaying for a SNP or a base mutation) in a target nucleic acid, methods of assaying for a target nucleic acid that lacks a PAM by amplifying the target nucleic acid to introduce a PAM, and compositions used in introducing a PAM via amplification into the target nucleic acid.

### Additional System Components

In some embodiments, systems include a package, carrier, or container that is compartmentalized to receive one or more containers such as vials, tubes, and the like, each of the container(s) comprising one of the separate elements to be used in a method described herein. Suitable containers include, for example, test wells, bottles, syringes, vials, and test tubes. In one embodiment, the containers are formed from a variety of materials such as glass, plastic, or polymers. The system or systems described herein contain packaging materials. Examples of packaging materials include, but are not limited to, pouches, blister packs, bottles, tubes, bags, containers, bottles, and any packaging material suitable for intended mode of usc.

A system may include labels listing contents and/or instructions for use, or package inserts with instructions for use. A set of instructions will also typically be included. In one embodiment, a label is on or associated with the container. In some embodiments, a label is on a container when letters, numbers or other characters forming the label are attached, molded, or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein. After packaging the formed product and wrapping or boxing to maintain a sterile barrier, the product may be terminally sterilized by heat sterilization, gas sterilization, gamma irradiation, or by electron beam sterilization. Alternatively, the product may be prepared and packaged by aseptic processing.

In some embodiments, systems comprise a solid support. An RNP or effector protein may be attached to a solid support. The solid support may be an electrode or a bead. The bead may be a magnetic bead. Upon cleavage, the RNP is liberated from the solid support and interacts with other mixtures. For example, upon cleavage of the nucleic acid of the RNP, the effector protein of the RNP flows through a chamber into a mixture comprising a substrate. When the effector protein meets the substrate, a reaction occurs, such as a colorimetric reaction, which is then detected. As another example, the protein is an enzyme substrate, and upon cleavage of the nucleic acid of the enzyme substrate-nucleic acid, the enzyme flows through a chamber into a mixture comprising the enzyme. When the enzyme substrate meets the enzyme, a reaction occurs, such as a calorimetric reaction, which is then detected.

### Certain System Conditions

In some embodiments, systems and methods are employed under certain conditions that enhance an activity of the effector protein relative to alternative conditions, as measured by a detectable signal released from cleavage of a reporter in the presence of the target nucleic acid. The detectable signal may be generated at about the rate of trans cleavage of a reporter nucleic acid. In some embodiments, the reporter nucleic acid is a homopolymeric reporter nucleic acid comprising 5 to 20 consecutive adenines (SEQ ID NO: 1492), 5 to 20 consecutive thymines (SEQ ID NO: 1493), 5 to 20 consecutive cytosines (SEQ ID NO: 1494), or 5 to 20 consecutive guanines (SEQ ID NO: 1495). In some embodiments, the reporter is an RNA-FQ reporter.

In some embodiments, effector proteins disclosed herein recognize, bind, or are activated by, different target nucleic acids having different sequences, but are active toward the same reporter nucleic acid, allowing for facile multiplexing in a single assay having a single ssRNA-FQ reporter.

In some embodiments, systems are employed under certain conditions that enhance trans cleavage activity of an effector protein. In some embodiments, under certain conditions, transcolatteral cleavage occurs at a rate of at least 0.005 mmol/min, at least 0.01 mmol/min, at least 0.05 mmol/min, at least 0.1 mmol/min, at least 0.2 mmol/min, at least 0.5 mmol/min, or at least 1 mmol/min. In some embodiments, systems and methods are employed under certain conditions that enhance cis-cleavage activity of the effector protein.

Certain conditions that may enhance the activity of an effector protein include a certain salt presence or salt concentration of the solution in which the activity occurs. For example, cis-cleavage activity of an effector protein may be inhibited or halted by a high salt concentration. The salt may be a sodium salt, a potassium salt, a calcium salt, a zince salt, a lithium salt, an ammonium salt, or a magnesium salt. In some embodiments, the salt is NaCl. In some embodiments, the salt is KNO3. In some embodiments, the salt is magnesium acetate. In some embodiments, the salt is magnesium chloride. In some embodiments, the salt is potassium acetate. In some embodiments, the salt is potassium nitrate. In some embodiments, the salt is zinc chloride. In embodiments, the salt is sodium chloride. In some embodiments, the salt is potassium chloride. In some embodiments, the salt is lithium acetate. In some embodiments, the salt is ammonium sulfate.

In some embodiments, the salt concentration is less than 150 mM, less than 125 mM, less than 100 mM, less than 75 mM, less than 50 mM, or less than 25 mM. In some embodiments, the salt concentration is more than 1 mM, but less than 150 mM, less than 125 mM, less than 100 mM, less than 75 mM, less than 50 mM, or less than 25 mM. In some embodiments, the salt concentration is more than 10 mM, but less than 150 mM, less than 125 mM, less than 100 mM, less than 75 mM, less than 50 mM, or less than 25 mM. In some embodiments, the salt is potassium acetate or sodium chloride and the concentration of salt in the solution is about 200 mM. In some embodiments, the salt is potassium acetate or, sodium chloride, lithium acetate, or ammonium sulfate and the concentration of salt in the solution is about 100 mM to about 200 mM.

Certain conditions that may enhance the activity of an effector protein include the pH of a solution in which the activity. For example, increasing pH may enhance trans cleavage activity. For example, the rate of trans cleavage activity may increase with increase in pH up to pH 9. In some embodiments, the pH is about 7, about 7.1, about 7.2, about 7.3, about 7.4, about 7.5, about 7.6, about 7.7, about 7.8, about 7.9, about 8, about 8.1, about 8.2, about 8.3, about 8.4, about 8.5, about 8.6, about 8.7, about 8.8, about 8.9, or about 9. In some embodiments, the pH is 7 to 7.5, 7.5 to 8, 8 to 8.5, 8.5 to 9, or 7 to 8.5. In some embodiments, the pH is less than 7. In some embodiments, the pH is greater than 7.

Certain conditions that may enhance the activity of an effector protein includes the temperature at which the activity is performed. In some embodiments, the temperature is about 25° C., to about 50° C. In some embodiments, the temperature is about 20° C., to about 40° C., about 30° C., to about 50° C., or about 40° C., to about 60° C. In some embodiments, the temperature is about 25° C., about 30° C., about 35° C., about 40° C., about 45° C., about 50° C., about 55° C., or about 60° C.

Certain conditions that may enhance the activity of an effector protein include the viscosity of a solution the effector protein is housed in. Compositions and systems described herein may comprise an engineered effector protein in a solution comprising a room temperature viscosity of less than about 15 centipoise, less than about 12 centipoise, less than about 10 centipoise, less than about 8 centipoise, less than about 6 centipoise, less than about 5 centipoise, less than about 4 centipoise, less than about 3 centipoise, less than about 2 centipoise, or less than about 1.5 centipoise.

Certain conditions that may enhance the activity of an effector protein include the ionic strength of the solution the effector protein is housed in. Compositions and systems may comprise an engineered effector protein in a solution comprising an ionic strength of less than about 500 mM, less than about 400 mM, less than about 300 mM, less than about 250 mM, less than about 200 mM, less than about 150 mM, less than about 100 mM, less than about 80 mM, less than about 60 mM, or less than about 50 mM. Compositions and systems may comprise an engineered effector protein and an assay excipient, which may stabilize a reagent or product, prevent aggregation or precipitation, or enhance or stabilize a detectable signal (e.g., a fluorescent signal). Examples of assay excipients include, but are not limited to, saccharides and saccharide derivatives (e.g., sodium carboxymethyl cellulose and cellulose acetate), detergents, glycols, polyols, esters, buffering agents, alginic acid, and organic solvents (e.g., DMSO).

### VIII. Methods of Detecting a Target Nucleic Acid

Provided herein are methods of detecting target nucleic acids. Methods may comprise detecting target nucleic acids with compositions or systems described herein. Methods may comprise detecting a target nucleic acid in a sample, e.g., a cell lysate, a biological fluid, or environmental sample. Methods may comprise detecting a target nucleic acid in a cell. In some embodiments, methods of detecting a target nucleic acid in a sample or cell comprises contacting the sample or cell with an effector protein or a multimeric complex thereof, a guide nucleic acid, wherein at least a portion of the guide nucleic acid is complementary to at least a portion of the target nucleic acid, and a reporter nucleic acid that is cleaved in the presence of the effector protein, the guide nucleic acid, and the target nucleic acid, and detecting a signal produced by cleavage of the reporter nucleic acid, thereby detecting the target nucleic acid in the sample. In some embodiments, methods result in trans cleavage of the reporter nucleic acid. In some embodiments, methods result in cis cleavage of the reporter nucleic acid. In some embodiments, methods of detecting a target nucleic acid include a reporter nucleic acid comprising a detectable moiety that produces a detectable signal in the presence of the target nucleic acid, the effector protein, and the guide nucleic acid.

In some embodiments, the methods of detecting a target nucleic acid comprising: a) contacting the target nucleic acid with a composition comprising an effector protein as described herein, a guide nucleic acid as described herein, and a reporter nucleic acid that is cleaved in the presence of the effector protein, the guide nucleic acid, and the target nucleic acid; and b) detecting a signal produced by cleavage of the reporter nucleic acid, thereby detecting the target nucleic acid in the sample. In some embodiments, the methods result in trans cleavage of the reporter nucleic acid. In some embodiments, the methods result in cis cleavage of the reporter nucleic acid. In some embodiments, the reporter nucleic acid is a single stranded nucleic acid. In some embodiments, the reporter comprises a detection moiety. In some embodiments, the reporter nucleic acid is capable of being cleaved by the effector protein. In some embodiments, a cleaved reporter nucleic acid generates a detectable product or a first detectable signal. In some embodiments, the first detectable signal is a change in color. In some embodiments, the change is color is measured indicating presence of the target nucleic acid. In some embodiments, the first detectable signal is measured on a support medium.

In some embodiments, methods of detecting comprise contacting a target nucleic acid, a cell comprising the target nucleic acid, or a sample comprising a target nucleic acid with an effector protein that comprises an amino acid sequence that is at least is at least 70%, at least 75%, at least 80%, at least 85%, at least 90%, at least 92%, at least 95%, at least 97%, at least 98%, at least 99%, or 100% identical to any one of the sequences of TABLE 1. In some embodiments, the amino acid sequence of the effector protein is at least 70%, at least 75%, at least 80%, at least 85%, at least 90%, at least 92%, at least 95%, at least 97%, at least 98%, at least 99%, or 100% identical to any one of the sequences of TABLE 1. In some embodiments, the effector protein comprises an amino acid sequence that is at least 90% identical to a sequence selected from any one of the sequences set forth in TABLE 1.

Methods may comprise contacting the sample to a complex comprising a guide nucleic acid comprising a segment that is reverse complementary to a segment of the target nucleic acid and a effector protein that exhibits sequence independent cleavage upon forming a complex comprising the segment of the guide nucleic acid binding to the segment of the target nucleic acid; and assaying for a signal indicating cleavage of at least some protein-nucleic acids of a population of protein-nucleic acids, wherein the signal indicates a presence of the target nucleic acid in the sample and wherein absence of the signal indicates an absence of the target nucleic acid in the sample.

Methods may comprise contacting the sample comprising the target nucleic acid with a guide nucleic acid targeting a target nucleic acid segment, an effector protein capable of being activated when complexed with the guide nucleic acid and the target nucleic acid segment, a single stranded nucleic acid of a reporter comprising a detection moiety, wherein the nucleic acid of a reporter is capable of being cleaved by the activated effector protein, thereby generating a first detectable signal, cleaving the single stranded nucleic acid of a reporter using the effector protein that cleaves as measured by a change in color, and measuring the first detectable signal on the support medium.

Methods may comprise contacting the sample or cell with an effector protein or a multimeric complex thereof and a guide nucleic acid in the presence of salts (e.g., compositions comprising salts). In some embodiments, the method may comprise a solution, wherein the solution comprises one or more salt. Accordingly, in some embodiments, the salt may be one or more salt selected from a magnesium salt, a zinc salt, a potassium salt, a calcium salt, and a sodium salt. In some embodiments, the salt is a combination of two or more salts. For example, in some embodiments, the salt is a combination of two or more salts selected from a magnesium salt, a zinc salt, a potassium salt, a calcium salt and a sodium salt. In some embodiments, the salt is magnesium acetate. In some embodiments, the salt is magnesium chloride. In some embodiments, the salt is potassium acetate. In some embodiments, the salt is potassium nitrate. In some embodiments, the salt is zinc chloride. In embodiments, the salt is sodium chloride. In some embodiments, the salt is potassium chloride. In some embodiments, the concentration of the one or more salt in the solution is about 0.001 mM to about 500 mM. In some embodiments, the concentration of the salt is about 0.001 mM to about 400 mM. In some embodiments, the concentration of the salt is about 0.001 mM to about 300 mM. In some embodiments, the concentration of the salt is about 0.001 mM to about 200 mM. In some embodiments, the concentration of the salt is about 0.001 mM to about 100 mM. In some embodiments, the concentration of the salt is about 0.001 mM to about 10 mM. In some embodiments, the concentration of the salt is about 0.01 mM to about 500 mM. In some embodiments, the concentration of the salt is about 0.01 mM to about 400 mM. In some embodiments, the concentration of the salt is about 0.01 mM to about 300 mM. In some embodiments, the concentration of the salt is about 0.01 mM to about 200 mM. In some embodiments, the concentration of the salt is about 0.01 mM to about 100 mM. In some embodiments, the concentration of the salt is about 0.01 mM to about 10 mM. In some embodiments, the concentration of the salt is about 0.1 mM to about 500 mM. In some embodiments, the concentration of the salt is about 0.1 mM to about 400 mM. In some embodiments, the concentration of the salt is about 0.1 mM to about 300 mM. In some embodiments, the concentration of the salt is about 0.1 mM to about 200 mM. In some embodiments, the concentration of the salt is about 0.1 mM to about 100 mM. In some embodiments, the concentration of the salt is about 0.1 mM to about 10 mM. In some embodiments, the concentration of the salt is about 1 mM to about 500 mM. In some embodiments, the concentration of the salt is about 1 mM to about 400 mM. In some embodiments, the concentration of the salt is about 1 mM to about 300 mM. In some embodiments, the concentration of the salt is about 1 mM to about 200 mM. In some embodiments, the concentration of the salt is about 1 mM to about 100 mM. In some embodiments, the concentration of the salt is about 1 mM to about 10 mM. In some embodiments, the concentration of the salt is about 10 mM to about 500 mM. In some embodiments, the concentration of the salt is about 10 mM to about 400 mM. In some embodiments, the concentration of the salt is about 10 mM to about 300 mM. In some embodiments, the concentration of the salt is about 10 mM to about 200 mM. In some embodiments, the concentration of the salt is about 10 mM to about 100 mM. In some embodiments, the concentration of the salt is about 100 mM to about 500 mM. In some embodiments, the concentration of the salt is about 100 mM to about 400 mM. In some embodiments, the concentration of the salt is about 100 mM to about 300 mM. In some embodiments, the concentration of the salt is about 100 mM to about 200 mM. In some embodiments, the salt is potassium acetate and the concentration of salt in the solution is about 100 mM. In some embodiments, the salt is potassium acetate or sodium chloride and the concentration of salt in the solution is about 200 mM. In some embodiments, the salt is potassium acetate or sodium chloride and the salt of potassium in the solution is about 100 mM to about 200 mM.

In some embodiments, methods of detecting a target nucleic acid by a cleavage assay. In some embodiments, the target nucleic acid is a single-stranded target nucleic acid. In some embodiments, the cleavage assay comprises: a) contacting the target nucleic acid with a composition comprising an effector protein as described; and b) cleaving the target nucleic acid. In some embodiments, the cleavage assay comprises an assay designed to visualize, quantitate or identify cleavage of a nucleic acid. In some embodiments, the method is an in vitro trans-cleavage assay. In some embodiments, a cleavage activity is a trans-cleavage activity. In some embodiments, the method is an in vitro cis-cleavage assay. In some embodiments, a cleavage activity is a cis-cleavage activity. In some embodiments, the cleavage assay follows a procedure comprising: (i) providing a composition comprising an equimolar amounts of an effector protein as described herein, and a guide nucleic acid described herein, under conditions to form an RNP complex; (ii) adding a plasmid comprising a target nucleic acid, wherein the target nucleic acid is a linear dsDNA, wherein the target nucleic acid comprises a target sequence and a PAM (iii) incubating the mixture under conditions to enable cleavage of the plasmid; (iv) quenching the reaction with EDTA and a protease; and (v) analyzing the reaction products (e.g., viewing the cleaved and uncleaved linear dsDNA with gel electrophoresis).

In some embodiments, there is a threshold of detection for methods of detecting target nucleic acids. In some embodiments, methods are not capable of detecting target nucleic acids that are present in a sample or solution at a concentration less than or equal to 10 nM. For example, when a threshold of detection is 10 nM, then a signal can be detected when a target nucleic acid is present in the sample at a concentration of 10 nM or more. In some embodiments, the threshold of detection is less than or equal to 5 nM, 1 nM, 0.5 nM, 0.1 nM, 0.05 nM, 0.01 nM, 0.005 nM, 0.001 nM, 0.0005 nM, 0.0001 nM. 0.00005 nM, 0.00001 nM, 10 pM, 1 pM, 500 fM, 250 fM, 100 fM, 50 fM, 10 fM, 5 fM, 1 fM, 500 attomole (aM), 100 aM, 50 aM, 10 aM, or 1 aM. In some embodiments, the threshold of detection is in a range of from 1 aM to 1 nM, 1 aM to 500 pM, 1 aM to 200 pM, 1 aM to 100 pM, 1 aM to 10 pM, 1 aM to 1 pM, 1 aM to 500 fM, 1 aM to 100 fM, 1 aM to 1 fM, 1 aM to 500 aM, 1 aM to 100 aM, 1 aM to 50 aM, 1 aM to 10 aM, 10 aM to 1 nM, 10 aM to 500 pM, 10 aM to 200 pM, 10 aM to 100 pM, 10 aM to 10 pM, 10 aM to 1 pM, 10 aM to 500 fM, 10 aM to 100 fM, 10 aM to 1 fM, 10 aM to 500 aM, 10 aM to 100 aM, 10 aM to 50 aM, 100 aM to 1 nM, 100 aM to 500 pM, 100 pM to 200 pM, 100 aM to 100 pM, 100 aM to 10 pM, 100 aM to 1 pM, 100 aM to 500 fM, 100 aM to 100 fM, 100 aM to 1 fM, 100 aM to 500 aM, 500 aM to 1 nM, 500 aM to 500 pM, 500 aM to 200 pM, 500 aM to 100 pM, 500 aM to 10 pM, 500 aM to 1 pM, 500 aM to 500 fM, 500 aM to 100 fM, 500 aM to 1 fM, 1 fM to 1 nM, 1 fM to 500 pM, 1 fM to 200 pM, 1 fM to 100 pM, 1 fM to 10 pM, 1 fM to 1 pM, 10 fM to 1 nM, 10 fM to 500 pM, 10 fM to 200 pM, 10 fM to 100 pM, 10 fM to 10 pM, 10 fM to 1 pM, 500 fM to 1 nM, 500 fM to 500 pM, 500 fM to 200 pM, 500 fM to 100 pM, 500 fM to 10 pM, 500 fM to 1 pM, 800 fM to 1 nM, 800 fM to 500 pM, 800 fM to 200 pM, 800 fM to 100 pM, 800 fM to 10 pM, 800 fM to 1 pM, 1 pM to 1 nM, 1 pM to 500 pM, 1 pM to 200 pM, 1 pM to 100 pM, or 1 pM to 10 pM, 1 n some embodiments, the threshold of detection in a range of from 800 fM to 100 μM, 1 pM to 10 μM, 10 fM to 500 fM, 10 fM to 50 fM, 50 fM to 100 fM, 100 fM to 250 fM, or 250 fM to 500 fM, 1 n some embodiments, the threshold of detection is in a range of from 2 aM to 100 μM, from 20 aM to 50 μM, from 50 aM to 20 μM, from 200 aM to 5 μM, or from 500 aM to 2 μM.

In some embodiments, the target nucleic acid is present in a cleavage reaction at a concentration of about 10 nM, about 20 nM, about 30 nM, about 40 nM, about 50 nM, about 60 nM, about 70 nM, about 80 nM, about 90 nM, about 100 nM, about 200 nM, about 300 nM, about 400 nM, about 500 nM, about 600 nM, about 700 nM, about 800 nM, about 900 nM, about 1 μM, about 10 μM, or about 100 μM. In some embodiments, the target nucleic acid is present in a cleavage reaction at a concentration of from 10 nM to 20 nM, from 20 nM to 30 nM, from 30 nM to 40 nM, from 40 nM to 50 nM, from 50 nM to 60 nM, from 60 nM to 70 nM, from 70 nM to 80 nM, from 80 nM to 90 nM, from 90 nM to 100 nM, from 100 nM to 200 nM, from 200 nM to 300 nM, from 300 nM to 400 nM, from 400 nM to 500 nM, from 500 nM to 600 nM, from 600 nM to 700 nM, from 700 nM to 800 nM, from 800 nM to 900 nM, from 900 nM to 1 μM, from 1 μM to 10 μM, from 10 μM to 100 μM, from 10 nM to 100 nM, from 10 nM to 1 μM, from 10 nM to 10 μM, from 10 nM to 100 μM, from 100 nM to 1 μM, from 100 nM to 10 μM, from 100 nM to 100 μM, or from 1 μM to 100 μM. In some embodiments, the target nucleic acid is present in a cleavage reaction at a concentration of from 20 nM to 50 μM, from 50 nM to 20 μM, or from 200 nM to 5 M.

In some embodiments, methods detect a target nucleic acid in less than 60 minutes. In some embodiments, methods detect a target nucleic acid in less than about 120 minutes, less than about 110 minutes, less than about 100 minutes, less than about 90 minutes, less than about 80 minutes, less than about 70 minutes, less than about 60 minutes, less than about 55 minutes, less than about 50 minutes, less than about 45 minutes, less than about 40 minutes, less than about 35 minutes, less than about 30 minutes, less than about 25 minutes, less than about 20 minutes, less than about 15 minutes, less than about 10 minutes, less than about 5 minutes, less than about 4 minutes, less than about 3 minutes, less than about 2 minutes, or less than about 1 minute.

In some embodiments, methods require at least about 120 minutes, at least about 110 minutes, at least about 100 minutes, at least about 90 minutes, at least about 80 minutes, at least about 70 minutes, at least about 60 minutes, at least about 55 minutes, at least about 50 minutes, at least about 45 minutes, at least about 40 minutes, at least about 35 minutes, at least about 30 minutes, at least about 25 minutes, at least about 20 minutes, at least about 15 minutes, at least about 10 minutes, or at least about 5 minutes to detect a target nucleic acid. In some embodiments, the sample is contacted with the reagents for from 5 minutes to 120 minutes, from 5 minutes to 100 minutes, from 10 minutes to 90 minutes, from 15 minutes to 45 minutes, or from 20 minutes to 35 minutes.

In some embodiments, methods of detecting are performed in less than 10 hours, less than 9 hours, less than 8 hours, less than 7 hours, less than 6 hours, less than 5 hours, less than 4 hours, less than 3 hours, less than 2 hours, less than 1 hour, less than 50 minutes, less than 45 minutes, less than 40 minutes, less than 35 minutes, less than 30 minutes, less than 25 minutes, less than 20 minutes, less than 15 minutes, less than 10 minutes, less than 9 minutes, less than 8 minutes, less than 7 minutes, less than 6 minutes, or less than 5 minutes. In some embodiments, methods of detecting are performed in about 5 minutes to about 10 hours, about 10 minutes to about 8 hours, about 15 minutes to about 6 hours, about 20 minutes to about 5 hours, about 30 minutes to about 2 hours, or about 45 minutes to about 1 hour.

Methods may comprise detecting a detectable signal within 5 minutes of contacting the sample and/or the target nucleic acid with the guide nucleic acid and/or the effector protein. In some embodiments, detecting occurs within 1, 2, 3, 4, 5, 6, 7, 8, 9, 10, 11, 12, 13, 14, 15, 20, 25, 30, 35, 40, 45, 50, 55, 60, 70, 80, 90, 100, 110, or 120 minutes of contacting the target nucleic acid. In some embodiments, detecting occurs within 1 to 120, 5 to 100, 10 to 90, 15 to 80, 20 to 60, or 30 to 45 minutes of contacting the target nucleic acid.

In some embodiments, methods of detecting as disclosed herein are compatible with methods for diagnosis of a disease or disorder.

### Amplification of a Target Nucleic Acid

Methods may comprise contacting the sample or cell with an effector protein or a multimeric complex thereof and a guide nucleic acid at a temperature of at least about 25° C., at least about 30° C., at least about 35° C., at least about 37° C., at least about 40° C., at least about 50° C., at least about 60° C., at least about 65° C., at least about 70° C., or at least about 75° C. In some embodiments, the temperature is not greater than 80° C. In some embodiments, the temperature is about 25° C., about 30° C., about 35° C., about 37° C., about 40° C., about 45° C., about 50° C., about 55° C., about 60° C., about 65° C., about 70° C., about 75° C., about 80° C., about 85° C., or about 90° C. In some embodiments, the temperature is about 25° C., to about 45° C., about 35° C., to about 55° C., about 37° C., to about 60° C., or about 55° C., to about 65° C. In some embodiments, the temperature is about 37° C., to about 45° C., about 37° C., to about 50° C., about 37° C., to about 55° C., about 37° C., to about 60° C., or about 37° C., to about 65° C.

Methods of detecting may comprise amplifying a target nucleic acid for detection using any of the compositions or systems described herein. Amplifying may comprise changing the temperature of the amplification reaction, also known as thermal amplification (e.g., PCR). Amplifying may be performed at essentially one temperature, also known as isothermal amplification. Amplifying may improve at least one of sensitivity, specificity, or accuracy of the detection of the target nucleic acid.

Amplifying may comprise subjecting a target nucleic acid to an amplification reaction selected from transcription mediated amplification (TMA), helicase dependent amplification (HDA), or circular helicase dependent amplification (cHDA), strand displacement amplification (SDA), recombinase polymerase amplification (RPA), loop mediated amplification (LAMP), exponential amplification reaction (EXPAR), rolling circle amplification (RCA), ligase chain reaction (LCR), simple method amplifying RNA targets (SMART), single primer isothermal amplification (SPIA), multiple displacement amplification (MDA), nucleic acid sequence based amplification (NASBA), hinge-initiated primer-dependent amplification of nucleic acids (HIP), nicking enzyme amplification reaction (NEAR), and improved multiple displacement amplification (IMDA). In some embodiments, amplifying may comprise subjecting a target nucleic acid to any one of the amplification methods described herein.

In some embodiments, amplification of the target nucleic acid comprises modifying the sequence of the target nucleic acid. For example, amplification may be used to insert a PAM sequence into a target nucleic acid that lacks a PAM sequence. In some embodiments, amplification may be used to increase the homogeneity of a target nucleic acid in a sample. For example, amplification may be used to remove a nucleic acid variation that is not of interest in the target nucleic acid.

Amplifying may take 1, 2, 3, 4, 5, 6, 7, 8, 9, 10, 11, 12, 13, 14, 15, 16, 17, 18, 19, 20, 25, 30, 40, 50, or 60 minutes. Amplifying may be performed at a temperature of around 20-45° C. Amplifying may be performed at a temperature of less than about 20° C., less than about 25° C., less than about 30° C. less than about 35° C., less than about 37° C., less than about 40° C., or less than about 45° C. The nucleic acid amplification reaction may be at a temperature of at least about 20° C., at least about 25° C., at least about 30° C., at least about 35° C., at least about 37° C., at least about 40° C., or at least about 45° C.

### Detection of a Target Nucleic Acid

Described herein are various methods of sample amplification and detection in a single reaction volume. Any of the devices described herein may be configured to perform amplification and detection in a same well, chamber, channel, or volume in the device. In some embodiments, methods include simultaneous amplification and detection in the same volume and/or in the same reaction. In some embodiments, methods include sequential amplification and detection in the same volume. In some embodiments, amplification and detection may occur in a single reaction, where reverse transcription, amplification, in vitro transcription, or any combination thereof, and detection are carried out in a single volume. Any suitable method of reverse transcription, amplification, in vitro transcription, and detection can be used in such a reaction, such as methods of reverse transcription, amplification, in vitro transcription, and detection described herein.

In some embodiments, a DETECTR reaction may be used to detect the presence of a specific target gene in the same. The DETECTR reaction may produce a detectable signal, as described elsewhere herein, in the presence of a target nucleic acid sequence comprising a target gene. The DETECTR reaction may not produce a signal in the absence of the target nucleic acid or in the presence of a nucleic acid sequence that does not comprise the specific mutation or comprises a different mutation. In some embodiments the mutation is a SNP. In some embodiments, a DETECTR reaction may comprise a guide RNA reverse complementary to a portion of a target nucleic acid sequence comprising a specific SNP. The guide RNA and the target nucleic acid comprising the specific SNP may bind to and activate a effector protein, thereby producing a detectable signal as described elsewhere herein. The guide RNA and a nucleic acid sequence that does not comprise the specific SNP may not bind to or activate the effector protein and may not produce a detectable signal. In some embodiments, a target nucleic acid sequence that may or may not comprise a specific SNP may be amplified using any amplification method disclosed herein. In some embodiments, the amplification reaction may be combined with a reverse transcription reaction, a DETECTR reaction, or both. In some embodiments, the target nucleic acid sequence can comprise a SNP. In some embodiments, the target nucleic acid sequence can comprise a sequence indicative of a human disease.

A DETECTR reaction, as described elsewhere herein, may produce a detectable signal specifically in the presence of a target nucleic acid sequence comprising a target gene. In some embodiments, the target nucleic acid sequence can comprise a sequence indicative of a human disease. In some embodiments, the detectable signal produced in the DETECTR reaction may be higher in the presence of a target nucleic acid comprising target nucleic acid than in the presence of a nucleic acid that does not comprise the target nucleic acid. In some embodiments, the DETECTR reaction may produce a detectable signal that is at least 1-fold, at least 2-fold, at least 3-fold, at least 4-fold, at least 5-fold, at least 10-fold, at least 20-fold, at least 30-fold, at least 40-fold, at least 50-fold, at least 100-fold, at least 200-fold, at least 300-fold, at last 400-fold, at least 500-fold, at least 1000-fold, at least 2000-fold, at least 3000-fold, at least 4000-fold, at least 5000-fold, at least 6000-fold, at least 7000-fold, at least 8000-fold, at least 9000-fold, at least 10000-fold, at least 50000-fold, at least 100000-fold, at least 500000-fold, or at least 1000000-fold greater in the presence of a target nucleic acid comprising a target nucleic acid than in the presence of a nucleic acid that does not comprise the target nucleic acid. In some embodiments, the DETECTR reaction may produce a detectable signal that is from 1-fold to 2-fold, from 2-fold to 3-fold, from 3-fold to 4-fold, from 4-fold to 5-fold, from 5-fold to 10-fold, from 10-fold to 20-fold, from 20-fold to 30-fold, from 30-fold to 40-fold, from 40-fold to 50-fold, from 50-fold to 100-fold, from 100-fold to 500-fold, from 500-fold to 1000-fold, from 1000-fold to 10,000-fold, from 10,000-fold to 100,000-fold, or from 100,000-fold to 1,000,000-fold greater in the presence of a target nucleic acid comprising a specific mutation or SNP than in the presence of a nucleic acid that does not comprise the specific mutation or SNP. In some embodiments, the target nucleic acid sequence can comprise a SNP. In some embodiments, the target nucleic acid sequence can comprise a sequence indicative of a human disease.

A DETECTR reaction may be used to detect the presence of a target nucleic acid associated with a disease or a condition in a nucleic acid sample. The DETECTR reaction may reach signal saturation within about 30 seconds, about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 75 minutes, about 80 minutes, or about 85 minutes and be used to detect the presence of a target gene associated with an increased likelihood of developing a disease or a condition in a nucleic acid sample. The DETECTR reaction may be used to detect the presence of a target gene associated with a phenotype in a nucleic acid sample. For example, a DETECTR reaction may be used to detect target nucleic acid, such as a gene or exon, or a mutation of a target nucleic acid, such as a SNP, as set forth in any one of TABLES 9, 9.1, 9.2, 9.3, and 9.4. In another example, a DETECTR reaction may be used to detect target nucleic acid or a mutation of a target nucleic acid associated with any one of the diseases or disorders recited in TABLE 10.

IX. Methods and Formulations for Introducing System Components and Compositions into a Target Cell

Disclosed herein, in some aspects, are systems and methods for introducing systems and components of such systems into a target cell. Such systems may comprise one or more components having any one of the effector proteins (or a nucleic acid comprising a nucleotide sequence encoding same) described herein. In some embodiments, such systems comprise one or more components having a guide nucleic acid (or a nucleic acid comprising a nucleotide sequence encoding same) described herein. In some embodiments, systems comprise one or more components having a guide nucleic acid and an additional nucleic acid. Systems and components thereof may be used to introduce effector proteins, guide nucleic acids, or combinations thereof into a target cell. Such methods may be used to modify or edit a target nucleic acid. In some embodiments, systems comprise an effector protein described herein, one or more guide nucleic acids, and a reagent for facilitating the introduction of the effector protein and the one or more guide nucleic acids. In some embodiments, system components for the methods comprise a solution, a buffer, a reagent for facilitating the introduction of the effector protein and the one or more guide nucleic acids, or combinations thereof. A guide nucleic acid (or a nucleic acid comprising a nucleotide sequence encoding same) and/or an effector protein (or a nucleic acid comprising a nucleotide sequence encoding same) described herein may be introduced into a host cell by any of a variety of well-known methods. As a non-limiting example, a guide nucleic acid and/or effector protein may be combined with a lipid. As another non-limiting example, a guide nucleic acid and/or effector protein may be combined with a particle or formulated into a particle.

### Methods for Introducing System Components and Compositions to a Host

Described herein are methods of introducing various components described herein to a host. A host may be any suitable host, such as a host cell. When described herein, a host cell may be an in vivo or in vitro eukaryotic cell, a prokaryotic cell (e.g., bacterial or archacal cell), or a cell from a multicellular organism (e.g., a cell line) cultured as a unicellular entity, which eukaryotic or prokaryotic cells may be, or have been, used as recipients for methods of introduction described herein, and include the progeny of the original cell which has been transformed by the methods of introduction described herein. It is understood that the progeny of a single cell may not necessarily be completely identical in morphology or in genomic or total DNA complement as the original parent, due to natural, accidental, or deliberate mutation. A host cell may be a recombinant host cell or a genetically modified host cell, if a heterologous nucleic acid, e.g., an expression vector, has been introduced into the cell.

Methods of introducing a nucleic acid and/or protein into a host cell are known in the art, and any convenient method may be used to introduce a subject nucleic acid (e.g., an expression construct/vector) into a target cell (e.g., a human cell, and the like). Suitable methods include, e.g., viral infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery (see, e.g., Panyam et al. Adv Drug Deliv Rev. 2012 Sep. 13, pii: S0169-409X (12) 00283-9, doi: 10.1016/j.addr.2012.09.023), and the like. In some embodiments, the nucleic acid and/or protein are introduced into a disease cell comprised in a pharmaceutical composition comprising the guide nucleic acid and/or effector protein and a pharmaceutically acceptable excipient, carrier or diluent.

In some embodiments, molecules of interest, such as nucleic acids of interest, are introduced to a host. In some embodiments, polypeptides, such as an effector protein are introduced to a host. In some embodiments, vectors, such as lipid particles and/or viral vectors may be introduced to a host. Introduction may be for contact with a host or for assimilation into the host, for example, introduction into a host cell.

In some embodiments, described herein are methods of introducing one or more nucleic acids, such as a nucleic acid encoding an effector protein, a nucleic acid that, when transcribed, produces an engineered guide nucleic acid, and/or a donor nucleic acid, or combinations thereof, into a host cell. Any suitable method may be used to introduce a nucleic acid into a cell. Suitable methods include, for example, viral infection, transfection, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct microinjection, nanoparticle-mediated nucleic acid delivery, and the like. Further methods are described throughout.

Introducing one or more nucleic acids into a host cell may occur in any culture media and under any culture conditions that promote the survival of the cells. Introducing one or more nucleic acids into a host cell may be carried out in vivo or ex vivo. Introducing one or more nucleic acids into a host cell may be carried out in vitro.

In some embodiments, an effector protein may be provided as RNA. The RNA may be provided by direct chemical synthesis or may be transcribed in vitro from a DNA (e.g., encoding the effector protein). Once synthesized, the RNA may be introduced into a cell by way of any suitable technique for introducing nucleic acids into cells (e.g., microinjection, electroporation, transfection, etc.). In some embodiments, introduction of one or more nucleic acid may be through the use of a vector and/or a vector system, accordingly, in some embodiments, compositions and system described herein comprise a vector and/or a vector system.

Vectors may be introduced directly to a host. In some embodiments, host cells may be contacted with one or more vectors as described herein, and in some embodiments, said vectors are taken up by the cells. Methods for contacting cells with vectors include but are not limited to electroporation, calcium chloride transfection, microinjection, lipofection, micro-injection, contact with the cell or particle that comprises a molecule of interest, or a package of cells or particles that comprise molecules of interest.

Components described herein may also be introduced directly to a host. For example, an engineered guide nucleic acid may be introduced to a host, specifically introduced into a host cell. Methods of introducing nucleic acids, such as RNA into cells include, but are not limited to direct injection, transfection, or any other method used for the introduction of nucleic acids.

Polypeptides (e.g., effector proteins) described herein may also be introduced directly to a host. In some embodiments, polypeptides described herein may be modified to promote introduction to a host. For example, polypeptides described herein may be modified to increase the solubility of the polypeptide. Such a polypeptide may optionally be fused to a polypeptide domain that increases solubility. The domain may be linked to the polypeptide through a defined protease cleavage site, such as TEV sequence which is cleaved by TEV protease. The linker may also include one or more flexible sequences, e.g, from 1 to 10 glycine residues. In some embodiments, the cleavage of the polypeptide is performed in a buffer that maintains solubility of the product, e.g, in the presence of from 0.5 to 2 M urea, in the presence of polypeptides and/or polynucleotides that increase solubility, and the like. Domains of interest include endosomolytic domains, e.g, influenza HA domain; and other polypeptides that aid in production, e.g. IF2 domain. GST domain. GRPE domain, and the like. In another example, the polypeptide may be modified to improve stability. For example, the polypeptides may be PEGylated, where the polyethyleneoxy group provides for enhanced lifetime in the blood stream. Polypeptides may also be modified to promote uptake by a host, such as a host cell. For example, a polypeptide described herein may be fused to a polypeptide permeant domain to promote uptake by a host cell. Any suitable permeant domains may be used in the non-integrating polypeptides of the present disclosure, including peptides, peptidomimetics, and non-peptide carriers. Examples include penetratin, a permeant peptide may be derived from the third alpha helix of Drosophila melanogaster transcription factor Antennapaedia; the HIV-1 tat basic region amino acid sequence, e.g., amino acids 49-57 of a naturally-occurring tat protein; and poly-arginine motifs, for example, the region of amino acids 34-56 of HIV-1 rev protein, nonaarginine, octa-arginine, and the like. The site at which the fusion is made may be selected in order to optimize the biological activity, secretion or binding characteristics of the polypeptide. The optimal site may be determined by suitable methods.

### Formulations for Introducing System Components and Compositions to a Host

Described herein are formulations of introducing compositions or components of a system described herein to a host. In some embodiments, such formulations, systems and compositions described herein comprise an effector protein and a carrier (e.g., excipient, diluent, vehicle, or filling agent). In some aspects of the present disclosure, the effector protein is provided in a pharmaceutical composition comprising the effector protein and any pharmaceutically acceptable excipient, carrier, or diluent.

X. Methods of Nucleic Acid Modification and/or Editing

Provided herein are methods, compositions, and systems of editing and/or modifying target nucleic acids. In general, editing refers to modifying the nucleobase sequence of a target nucleic acid. Modifying may refer to generally changing the physical composition of a target nucleic acid. However, compositions and systems disclosed herein may also be capable of making epigenetic modifications of target nucleic acids. In some embodiments, epigenetic modifications of target nucleic acids, do not change the nucleotide sequence of the target nucleic acids per se. Effector proteins, multimeric complexes thereof and systems described herein may be used for editing or modifying a target nucleic acid. Editing a target nucleic acid may comprise one or more of cleaving the target nucleic acid, deleting one or more nucleotides of the target nucleic acid, inserting one or more nucleotides into the target nucleic acid, substituting one or more nucleotides of the target nucleic acid, mutating one or more nucleotides of the target nucleic acid, modifying (e.g., methylating, demethylating, deaminating, or oxidizing), or otherwise changing of one or more nucleotides of the target nucleic acid.

Compositions, methods, and systems described herein may modify a coding portion of a gene, a non-coding portion of a gene, or a combination thereof. Modifying at least one gene using the compositions, methods or systems described herein may reduce or increase expression of one or more genes. In some embodiments, the compositions, methods or systems reduce expression of one or more genes by at least 10%, at least 20%, at least 30%, at least 40%, at least 50%, at least 60%, at least 70%, at least 80%, at least 90%, or at least 95%. In some embodiments, the compositions, methods or systems remove all expression of a gene, also referred to as genetic knock out. In some embodiments, the compositions, methods or systems increase expression of one or more genes by at least 10%, at least 20%, at least 30%, at least 40%, at least 50%, at least 60%, at least 70%, at least 80%, at least 90%, or at least 100%.

In some embodiments, the compositions, methods or systems comprise a nucleic acid expression vector, or use thereof, to introduce an effector protein, guide nucleic acid, donor template or any combination thereof to a cell. In some embodiments, the nucleic acid expression vector is a viral vector. Viral vectors include, but are not limited to, retroviruses, adenoviruses, adeno-associated viruses, and herpes simplex viruses. In some embodiments, the viral vector is a replication-defective viral vector, comprising an insertion of a therapeutic gene inserted in genes essential to the lytic cycle, preventing the virus from replicating and exerting cytotoxic effects. In some embodiments, the viral vector is an adeno associated viral (AAV) vector. In some embodiments, the nucleic acid expression vector is a non-viral vector. In some embodiments, compositions and methods comprise a lipid, polymer, nanoparticle, or a combination thereof, or use thereof, to introduce an effector protein (e.g., Cas protein), guide nucleic acid, donor template or any combination thereof to a cell. Non-limiting examples of lipids and polymers are cationic polymers, cationic lipids, or bio-responsive polymers. In some embodiments, the bio-responsive polymer exploits chemical-physical properties of the endosomal environment (e.g., pH) to preferentially release the genetic material in the intracellular space.

Methods of editing may comprise contacting a target nucleic acid with an effector protein described herein and a guide nucleic acid, wherein the effector protein comprises an amino acid sequence that is at least 70%, at least 75%, at least 80%, at least 85%, at least 90%, at least 95%, at least 97%, or at least 98%, at least 99%, or 100% identical to the sequence set forth in TABLE 1. In some embodiments, the effector protein comprises an amino acid sequence that is at least 70%, at least 75%, at least 80%, at least 85%, at least 90%, at least 95%, at least 97%, or at least 98%, or at least 99% similar to the sequence set forth in TABLE 1. In some instances, the guide nucleic acid comprises a spacer sequence that is at least 70%, at least 75%, at least 80%, at least 85%, at least 90%, at least 92%, at least 95%, or 100% identical to any one of the sequences set forth in TABLE 4 and a repeat sequence that is at least 70%, at least 75%, at least 80%, at least 85%, at least 90%, at least 92%, at least 95%, or 100% identical to the sequence set forth in TABLE 5. In some instances, the nucleobase sequence of the guide nucleic acid is at least 70%, at least 75%, at least 80%, at least 85%, at least 90%, at least 92%, at least 95%, at least 97%, at least 98%, at least 99%, or 100% identical to any one of the gRNA sequences set forth in TABLE 6.

Methods of modifying may comprise contacting a target nucleic acid with one or more components, compositions or systems described herein. In some embodiments, a method of modifying comprises contacting a target nucleic acid with at least one of: a) one or more effector proteins, or one or more nucleic acids encoding one or more effector proteins; or b) one or more guide nucleic acids, or one or more nucleic acids encoding one or more guide nucleic acids. In some embodiments, a method of modifying comprises contacting a target nucleic acid with a system described herein wherein the system comprises components comprising at least one of: a) one or more effector proteins, or one or more nucleic acids encoding one or more effector proteins; or b) one or more guide nucleic acids, or one or more nucleic acids encoding one or more guide nucleic acids. In some embodiments, a method of modifying comprises contacting a target nucleic acid with a composition described herein comprising at least one of: a) one or more effector proteins, or one or more nucleic acids encoding one or more effector proteins; or b) one or more guide nucleic acids, or one or more nucleic acids encoding one or more guide nucleic acids; in a composition. In some embodiments, a method of modifying as described herein produces a modified target nucleic acid.

Editing may introduce a mutation (e.g., point mutations, deletions) in a target nucleic acid relative to a corresponding wildtype nucleobase sequence. Editing may remove or correct a disease-causing mutation in a nucleic acid sequence to produce a corresponding wildtype nucleotide sequence. Editing may remove/correct point mutations, deletions, null mutations, or tissue-specific mutations in a target nucleic acid. Editing may be used to generate gene knock-out, gene knock-in, gene editing, gene tagging, or a combination thereof. Methods of the disclosure may be targeted to any locus in a genome of a cell.

Editing, and generally modifying, may comprise single stranded cleavage, double stranded cleavage, donor nucleic acid insertion, epigenetic modification (e.g., methylation, demethylation, acetylation, or deacetylation), or a combination thereof. In some instances, cleavage (single-stranded or double-stranded) is site-specific, meaning cleavage occurs at a specific site in the target nucleic acid, often within the region of the target nucleic acid that hybridizes with the guide nucleic acid spacer region. In some cases, the effector proteins introduce a single-stranded break in a target nucleic acid to produce a cleaved nucleic acid. In some cases, the effector protein is capable of introducing a break in a single stranded RNA (ssRNA). The effector protein may be coupled to a guide nucleic acid that targets a particular region of interest in the ssRNA. In some instances, the target nucleic acid, and the resulting cleaved nucleic acid is contacted with a nucleic acid for homologous recombination (e.g., homology directed repair (HDR)) or non-homologous end joining (NHEJ). In some cases, a double-stranded break in the target nucleic acid may be repaired (e.g., by NHEJ or HDR) without insertion of a donor template, such that the repair results in an indel in the target nucleic acid at or near the site of the double-stranded break. In some embodiments, an indel, sometimes referred to as an insertion-deletion or indel mutation, is a type of genetic mutation that results from the insertion and/or deletion of nucleotides in a target nucleic acid. An indel can vary in length (e.g., 1 to 1,000 nucleotides in length) and be detected using methods well known in the art, including sequencing. If the number of nucleotides in the insertion/deletion is not divisible by three, and it occurs in a protein coding region, it is also a frameshift mutation. Indel percentage is the percentage of sequencing reads that show at least one nucleotide has been mutation that results from the insertion and/or deletion of nucleotides regardless of the size of insertion or deletion, or number of nucleotides mutated. For example, if there is at least one nucleotide deletion detected in a given target nucleic acid, it counts towards the percent indel value. As another example, if one copy of the target nucleic acid has one nucleotide deleted, and another copy of the target nucleic acid has 10 nucleotides deleted, they are counted the same. This number reflects the percentage of target nucleic acids that are edited by a given effector protein.

In some embodiments, methods of modifying described herein cleave a target nucleic acid at one or more locations to generate a cleaved target nucleic acid. In some embodiments, the cleaved target nucleic acid undergoes recombination (e.g., NHEJ or HDR). In some embodiments, cleavage in the target nucleic acid may be repaired (e.g., by NHEJ or HDR) without insertion of a donor nucleic acid, such that the repair results in an indel in the target nucleic acid at or near the site of the cleavage site. In some embodiments, cleavage in the target nucleic acid may be repaired (e.g., by NHEJ or HDR) with insertion of a donor nucleic acid, such that the repair results in an indel in the target nucleic acid at or near the site of the cleavage site.

In some instances, wherein the compositions, systems, and methods of the present disclosure comprise an additional guide nucleic acid or a use thereof, the dual-guided compositions, systems, and methods described herein can modify the target nucleic acid in two locations. In some cases, dual-guided editing can comprise cleavage of the target nucleic acid in the two locations targeted by the guide RNAs. In some embodiments, modification of the target nucleic acid between the hybridized guide nucleic acids may result in deletion (or removal) of the nucleic acid between the hybridized guide nucleic acids, insertion of a nucleic acid, such as a donor nucleic acid, between the hybridized guide nucleic acids, substitution of a nucleic acid between the hybridized guide nucleic acids, and the like. In some embodiments, upon modification of the sequence between the guide nucleic acids, the wild-type reading frame is restored. In certain embodiments, upon removal of the sequence between the guide nucleic acids, the wild-type reading frame is restored. A wild-type reading frame can be a reading frame that produces at least a partially, or fully, functional protein. A non-wild-type reading frame can be a reading frame that produces a non-functional or partially non-functional protein.

Accordingly, in some embodiments, compositions, systems, and methods described herein can edit 1 to 1,000 nucleotides or any integer in between, in a target nucleic acid. In certain embodiments. 1 to 1,000, 2 to 900, 3 to 800, 4 to 700, 5 to 600, 6 to 500, 7 to 400, 8 to 300, 9 to 200, or 10 to 100 nucleotides, or any integer in between, can be edited by the compositions, systems, and methods described herein. In some embodiments, 1, 2, 3, 4, 5, 6, 7, 8, 9, 10, or more nucleotides can be edited by the compositions, systems, and methods described herein. In some embodiments, 10, 20, 30, 40, 50, 60, 70, 80 90, 100 or more nucleotides, or any integer in between, can be edited by the compositions, systems, and methods described herein. In some embodiments, 100, 200, 300, 400, 500, 600, 700, 800, 900 or more nucleotides, or any integer in between, can be edited by the compositions, systems, and methods described herein.

In some instances, the effector protein is fused to a chromatin-modifying enzyme. In some cases, the fusion protein chemically modifies the target nucleic acid, for example by methylating, demethylating, or acetylating the target nucleic acid in a sequence specific or non-specific manner.

Methods may comprise use of two or more effector proteins. An illustrative method for introducing a break in a target nucleic acid comprises contacting the target nucleic acid with: (a) a first engineered guide nucleic acid comprising a region that binds to a first effector protein, wherein the effector protein comprises at least 75% sequence identity to any one of the sequences of TABLE 1; and (b) a second engineered guide nucleic acid comprising a region that binds to a second effector protein, wherein the effector protein comprises at least 75% sequence identity to any one of the sequences of TABLE 1, wherein the first engineered guide nucleic acid comprises an additional region that binds to the target nucleic acid and wherein the second engineered guide nucleic acid comprises an additional region that binds to the target nucleic acid. In some instances, the nucleobase sequence of the guide nucleic acid is at least 70%, at least 75%, at least 80%, at least 85%, at least 90%, at least 92%, at least 95%, at least 97%, at least 98%, at least 99%, or 100% identical to any one of any one of the gRNA sequences of TABLE 6. In some instances, the guide nucleic acid comprises a crRNA sequence comprising a spacer sequence that is at least 70%, at least 75%, at least 80%, at least 85%, at least 90%, at least 92%, at least 95%, or 100% identical to any one of the sequences of TABLE 4 and a repeat sequence that is at least 70%, at least 75%, at least 80%, at least 85%, at least 90%, at least 92%, at least 95%, or 100% identical to the sequence of TABLE 5. In some embodiments, the first and second effector protein are identical. In some embodiments, the first and second effector protein are not identical.

In some instances, editing a target nucleic acid comprises genome editing. Genome editing may comprise modifying a genome, chromosome, plasmid, or other genetic material of a cell or organism. In some instances, the genome, chromosome, plasmid, or other genetic material of the cell or organism is modified in vivo. In some instances, the genome, chromosome, plasmid, or other genetic material of the cell or organism is modified in a cell. In some instances, the genome, chromosome, plasmid, or other genetic material of the cell or organism is modified in vitro. For example, a plasmid may be modified in vitro using a composition described herein and introduced into a cell or organism. In some instances, modifying a target nucleic acid may comprise deleting a sequence from a target nucleic acid. For example, a mutated sequence or a sequence associated with a disease may be removed from a target nucleic acid. In some instances, modifying a target nucleic acid may comprise replacing a sequence in a target nucleic acid with a second sequence. For example, a mutated sequence or a sequence associated with a disease may be replaced with a second sequence lacking the mutation or that is not associated with the disease. In some embodiments, editing a target nucleic acid may comprise deleting or replacing a sequence comprising markers associated with a disease or disorder. In some instances, modifying a target nucleic acid may comprise introducing a sequence into a target nucleic acid. For example, a beneficial sequence or a sequence that may reduce or eliminate a disease may be inserted into the target nucleic acid.

In some instances, methods comprise inserting a donor nucleic acid into a cleaved target nucleic acid. The donor nucleic acid may be inserted at a specified (e.g., effector protein targeted) point within the target nucleic acid. In some embodiments, the cleaved target nucleic acid is cleaved at a single location. In such embodiments, the methods comprise contacting a target nucleic acid with an effector protein described herein, thereby introducing a single-stranded break in the target nucleic acid; and contacting the target nucleic acid with a donor nucleic acid for homologous recombination, optionally by HDR or NHEJ, thereby introducing a new sequence into the target nucleic acid (e.g., at a cleavage site). In some embodiments, the cleaved target nucleic acid is cleaved at two locations. In some instances, methods comprise contacting a target nucleic acid with an effector protein comprising an amino acid sequence that is at least 70%, at least 75%, at least 80%, at least 85%, at least 90%, at least 95%, at least 97%, at least 98%, at least 99%, or 100% identical to any one of the sequences of TABLE 1, thereby introducing a single-stranded break in the target nucleic acid; contacting the target nucleic acid with a second effector protein, optionally comprising an amino acid sequence that is at least 70%, at least 75%, at least 80%, at least 85%, at least 90%, at least 95%, at least 97%, at least 98%, or at least 99% identical to any one of the sequences of TABLE 1, to generate a second cleavage site in the target nucleic acid, ligating the regions flanking the first and second cleavage site, optionally through NHEJ or single-strand annealing, thereby resulting in the excision of a portion of the target nucleic acid between the first and second cleavage sites from the target nucleic acid; and contacting the target nucleic acid with a donor nucleic acid for homologous recombination, optionally via HDR or NHEJ, thereby introducing a new sequence into the target nucleic acid (e.g., at a cleavage site or in between two cleavage sites).

In some cases, methods comprise editing a target nucleic acid with two or more effector proteins. Editing a target nucleic acid may comprise introducing a two or more single-stranded breaks in a target nucleic acid. In some instances, a break may be introduced by contacting a target nucleic acid with an effector protein and a guide nucleic acid. The guide nucleic acid may bind to the effector protein and hybridize to a region of the target nucleic acid, thereby recruiting the effector protein to the region of the target nucleic acid. Binding of the effector protein to the guide nucleic acid and the region of the target nucleic acid may activate the effector protein, and the effector protein may introduce a break (e.g., a single stranded break) in the region of the target nucleic acid. In some instances, modifying a target nucleic acid may comprise introducing a first break in a first region of the target nucleic acid and a second break in a second region of the target nucleic acid. For example, modifying a target nucleic acid may comprise contacting a target nucleic acid with a first guide nucleic acid that binds to a first effector protein and hybridizes to a first region of the target nucleic acid and a second guide nucleic acid that binds to a second programmable nickase and hybridizes to a second region of the target nucleic acid. The first effector protein may introduce a first break in a first strand at the first region of the target nucleic acid, and the second effector protein may introduce a second break in a second strand at the second region of the target nucleic acid. In some instances, a segment of the target nucleic acid between the first break and the second break may be removed, thereby modifying the target nucleic acid. In some instances, a segment of the target nucleic acid between the first break and the second break may be replaced (e.g., with donor nucleic acid), thereby modifying the target nucleic acid. In some instances, the effector protein comprises an amino acid sequence that is at least 70%, at least 75%, at least 80%, at least 85%, at least 90%, at least 95%, at least 97%, at least 98%, at least 99%, or 100% identical to any one of the sequences of TABLE 1. In some instances, the nucleobase sequence of the guide nucleic acid is at least 70%, at least 75%, at least 80%, at least 85%, at least 90%, at least 92%, at least 95%, at least 97%, at least 98%, at least 99%, or 100% identical to any one of the gRNA sequences of TABLE 6. In some instances, the guide nucleic acid comprises a crRNA sequence comprising a spacer sequence that is at least 70%, at least 75%, at least 80%, at least 85%, at least 90%, at least 95%, or 100% identical to any one of the sequences of TABLE 4 and a repeat sequence that is at least 70%, at least 75%, at least 80%, at least 85%, at least 90%, at least 92%, at least 95%, or 100% identical to the sequence of TABLE 5.

In some cases, editing is achieved by fusing an effector protein to a heterologous sequence. The heterologous sequence may be a suitable fusion partner, e.g., a protein that provides recombinase activity by acting on the target nucleic acid. In some instances, the fusion protein comprises an effector protein fused to a heterologous sequence by a linker. The heterologous sequence or fusion partner may be a base editing domain. The base editing domain may be an ADAR1/2 or any functional variant thereof. The heterologous sequence or fusion partner may be fused to the C-terminus. N-terminus, or an internal portion (e.g., a portion other than the N- or C-terminus) of the effector protein. The heterologous sequence or fusion partner may be fused to the effector protein by a linker. A linker may be a peptide linker or a non-peptide linker. In some instances, the linker is an XTEN linker. In some instances, the linker comprises one or more repeats a tri-peptide GGS. In some instances, the linker is from 1 to 100 amino acids in length. In some instances, the linker is more 100 amino acids in length. In some instances, the linker is from 10 to 27 amino acids in length. A non-peptide linker may be a polyethylene glycol (PEG), polypropylene glycol (PPG), co-poly(ethylene/propylene) glycol, polyoxyethylene (POE), polyurethane, polyphosphazene, polysaccharides, dextran, polyvinyl alcohol, polyvinylpyrrolidones, polyvinyl ethyl ether, polyacrylamide, polyacrylate, polycyanoacrylates, lipid polymers, chitins, hyaluronic acid, heparin, or an alkyl linker.

Methods, systems and compositions described herein can edit or modify a target nucleic acid wherein such editing or modification can effect one or more indels. In some embodiments, where compositions, systems, and/or methods described herein effect one or more indels, then in certain embodiments, the impact on the transcription and/or translation of the target nucleic acid can be predicted depending on: 1) the amount of indels generated; and 2) the location of the indel on the target nucleic acid. For example, as described herein, in certain embodiments, if the amount of indels is not divisible by three, and the indels occur within or along a protein coding region, then the modification or mutation can be a frameshift mutation.

In certain embodiments, if the amount of indels is divisible by three, then a frameshift mutation may not be effected, but a splicing disruption mutation and/or sequence skip mutation may be effected, such as an exon skip mutation. In certain embodiments, if the amount of indels is not evenly divisible by three, then a frameshift mutation may be effected.

Methods, systems and compositions described herein can edit or modify a target nucleic acid wherein such editing or modification can be measured by indel activity. Indel activity measures the amount of change in a target nucleic acid (e.g., nucleotide deletion(s) and/or insertion(s)) compared to a target nucleic acid that has not been contacted by a polypeptide described in compositions, systems, and methods described herein. For example, indel activity can be detected by next generation sequencing of one or more target loci of a target nucleic acid where indel percentage is calculated as the fraction of sequencing reads containing insertions or deletions relative to an unedited reference sequence. In certain instances, methods, systems, and compositions comprising an effector protein and guide nucleic acid described herein can exhibit about 0.0001% to about 65% or more indel activity upon contact to a target nucleic acid compared to a target nucleic acid non-contacted with compositions, systems, or by methods described herein. For example, methods, systems, and compositions comprising an effector protein and guide nucleic acid described herein can exhibit about 0.0001%, about 0.001%, about 0.01%, about 0.1%, about 1%, about 5%, about 10%, about 15%, about 20%, about 25%, about 30%, about 35%, about 40%, about 45%, about 50%, about 55%, about 60%, about 65% or more indel activity.

In some embodiments, editing or modifications of a target nucleic acid as described herein effects one or more mutations comprising splicing disruption mutations, frameshift mutations (e.g., 1+ or 2+ frameshift mutation), sequence deletion, sequence skipping, sequence reframing, sequence knock-in, or any combination thereof.

A splicing disruption can be a modification that disrupts the splicing of a target nucleic acid or splicing of a sequence that is transcribed from a target nucleic acid relative to a target nucleic acid without the splicing disruption.

A frameshift mutation can be a modification that alters the reading frame of a target nucleic acid relative to a target nucleic acid without the frameshift mutation. In certain embodiments, a frameshift mutation can be a +2 frameshift mutation wherein a reading frame is modified by 2 bases. In certain embodiments, a frameshift mutation can be a +1 frameshift mutation wherein a reading frame is modified by 1 base. In certain embodiments, a frameshift mutation is a modification that alters the number of bases in a target nucleic acid so that it is not divisible by three. In some embodiments, a frameshift mutation can be a modification that is not a splicing disruption.

A sequence as described in reference to a sequence deletion, sequence skipping, sequence reframing, and sequence knock-in can be a DNA sequence, a RNA sequence, a modified DNA or RNA sequence, a mutated sequence, a wild-type sequence, a coding sequence, a non-coding sequence, an exonic sequence (exon), an intronic sequence (intron), or any combination thereof. Such a sequence can be a sequence that is associated with a disease as described herein, such as DMD.

In certain embodiments, sequence deletion is a modification where one or more sequences in a target nucleic acid are deleted relative to a target nucleic acid without the sequence deletion. In certain embodiments, a sequence deletion can be a splicing disruption or a frameshift mutation. In certain embodiments, a sequence deletion can be a splicing disruption.

In certain embodiments, sequence skipping is a modification where one or more sequences in a target nucleic acid are skipped upon transcription or translation of the target nucleic acid relative to a target nucleic acid without the sequence skipping. In certain embodiments, sequence skipping can result in or effect a splicing disruption or a frameshift mutation. In certain embodiments, sequence skipping can result in or effect a splicing disruption.

In certain embodiments, sequence reframing is a modification where one or more bases in a target are modified so that the reading frame of the sequence is reframed relative to a target nucleic acid without the sequence reframing. In certain embodiments, sequence reframing can result in or effect a splicing disruption or a frameshift mutation. In certain embodiments, sequence reframing can result in or effect a frameshift mutation.

In certain embodiments, sequence knock-in is a modification where one or more sequences is inserted into a target nucleic acid relative to a target nucleic acid without the sequence knock-in. In certain embodiments, sequence knock-in can result in or effect a splicing disruption or a frameshift mutation. In certain embodiments, sequence knock-in can result in or effect a splicing disruption.

In certain embodiments, editing or modification of a target nucleic acid can be locus specific, wherein compositions, systems, and methods described herein can edit or modify a target nucleic acid at one or more specific loci to effect one or more specific mutations comprising splicing disruption mutations, frameshift mutations, sequence deletion, sequence skipping, sequence reframing, sequence knock-in, or any combination thereof. For example, editing or modification of a specific locus can effect any one of a splicing disruption, frameshift (e.g., 1+ or 2+ frameshift), sequence deletion, sequence skipping, sequence reframing, sequence knock-in, or any combination thereof. In certain embodiments, editing or modification of a target nucleic acid can be locus specific, modification specific, or both. In certain embodiments, editing or modification of a target nucleic acid can be locus specific, modification specific, or both, wherein compositions, systems, and methods described herein comprise an effector protein described herein and a guide nucleic acid described herein.

Methods of editing a target nucleic acid or modulating the expression of a target nucleic acid may be performed in vivo. Methods of editing a target nucleic acid or modulating the expression of a target nucleic acid may be performed in vitro. For example, a plasmid may be modified in vitro using a composition described herein and introduced into a cell or organism. Methods of editing a target nucleic acid or modulating the expression of a target nucleic acid may be performed ex vivo. For example, methods may comprise obtaining a cell from a subject, modifying a target nucleic acid in the cell with methods described herein, and returning the cell to the subject.

In some embodiments, methods of modifying described herein comprise contacting a target nucleic acid with one or more components, compositions or systems described herein. In some embodiments, the one or more components, compositions or systems described herein comprise at least one of: a) one or more effector proteins, or one or more nucleic acids encoding one or more effector proteins; and b) one or more guide nucleic acids, or one or more nucleic acids encoding one or more guide nucleic acids. In some embodiments, the one or more effector proteins introduce a single-stranded break or a double-stranded break in the target nucleic acid. In some embodiments, methods of modifying described herein produce a modified target nucleic acid comprising an engineered nucleic acid sequence that expresses polypeptide having new activity as compared to an unmodified target nucleic acid, or alters expression of an endogenous polypeptide as compared to an unmodified target nucleic acid.

In some embodiments, methods of modifying described herein comprise using one or more guide nucleic acids or uses thereof, wherein the methods modify a target nucleic acid at a single location. In some embodiments, the methods comprise contacting an RNP comprising an effector protein and a guide nucleic acid to the target nucleic acid. In some embodiments, the methods introduce a mutation (e.g., point mutations, deletions) in the target nucleic acid relative to a corresponding wildtype nucleotide sequence. In some embodiments, the methods remove or correct a disease-causing mutation in a nucleic acid sequence to produce a corresponding wildtype nucleotide sequence. In some embodiments, the methods remove/correct point mutations, deletions, null mutations, or tissue-specific mutations in a target nucleic acid. In some embodiments, the methods introduce a single stranded cleavage, a nick, a deletion of one or two nucleotides, an insertion of one or two nucleotides, a substitution of one or two nucleotides, an epigenetic modification (e.g., methylation, demethylation, acetylation, or deacetylation), or a combination thereof to the target nucleic acid. In some embodiments, the methods comprise using an effector protein and two guide nucleic acids, wherein two RNPs cleave the target nucleic acid at the same location, wherein a first RNP comprises the effector protein and a first guide nucleic acid, and wherein a second RNP comprises the effector protein and a second guide nucleic acid. In some embodiments, methods comprising using two effector protein and two guide nucleic acids, wherein both RNPs cleave the target nucleic acid at the same location, wherein a first RNP comprises a first effector protein and a first target nucleic acid, and wherein a second RNP comprises a second effector protein and a second target nucleic acid.

In some embodiments, methods of modifying described herein comprise using one or more guide nucleic acids or uses thereof, wherein the methods modify a target nucleic acid at two different locations. In some embodiments, the methods introduce two cleavage sites in the target nucleic acid, wherein a first cleavage site and a second cleavage site comprise one or more nucleotides therebetween. In some embodiments, the methods cause deletion of the one or more nucleotides. In some embodiments, the deletion restores a wild-type reading frame. In some embodiments, the wild-type reading frame produces at least a partially functional protein. In some embodiments, the deletion causes a non-wild-type reading frame. In some embodiments, a non-wild-type reading frame produces a partially functional protein or non-functional protein. In some embodiments, the at least partially functional protein has at least 50%, at least 60%, at least 70%, at least 80%, at least 90%, at least 100%, at least 110%, at least 120%, at least 130%, at least 140%, at least 150%, at least 180%, at least 200%, at least 300%, at least 400% activity compared to a corresponding wildtype protein. In some embodiments, the methods comprise using an effector protein and two guide nucleic acids, wherein two RNPs cleave the target nucleic acid at different locations, wherein a first RNP comprises the effector protein and a first guide nucleic acid, and wherein a second RNP comprises the effector protein and a second guide nucleic acid. In some embodiments, methods comprising using two effector protein and two guide nucleic acids, wherein both RNPs cleave the target nucleic acid at the same location, wherein a first RNP comprises a first effector protein and a first target nucleic acid, and wherein a second RNP comprises a second effector protein and a second target nucleic acid.

In some embodiments, methods of editing described herein comprise inserting a donor nucleic acid into a cleaved target nucleic acid. In some embodiments, the cleaved target nucleic acid formed by introducing a single-stranded break into a target nucleic acid. The donor nucleic acid may be inserted at a specified (e.g., effector protein targeted) point within the target nucleic acid. In some embodiments, the cleaved target nucleic acid is cleaved at a single location. In such embodiments, the methods comprise contacting a target nucleic acid with an effector protein described herein, thereby introducing a single-stranded break in the target nucleic acid; and contacting the target nucleic acid with a donor nucleic acid for homologous recombination, optionally by HDR or NHEJ, thereby introducing a new sequence into the target nucleic acid (e.g., at a cleavage site). In some embodiments, the cleaved target nucleic acid is cleaved at two locations. In such embodiments, the methods comprise contacting a target nucleic acid with an effector protein described herein, thereby introducing a single-stranded break in the target nucleic acid; contacting the target nucleic acid with a second effector protein described herein, to generate a second cleavage site in the target nucleic acid, ligating the regions flanking the first and second cleavage site, optionally through NHEJ or single-strand annealing, thereby resulting in the excision of a portion of the target nucleic acid between the first and second cleavage sites from the target nucleic acid; and contacting the target nucleic acid with a donor nucleic acid for homologous recombination, optionally by HDR or NHEJ, thereby introducing a new sequence into the target nucleic acid (e.g., in between two cleavage sites).

### Donor Nucleic Acids

In some embodiments, a donor nucleic acid comprises a nucleic acid that is incorporated into a target nucleic acid or genome. In some embodiments, a donor nucleic acid comprises a nucleotide or a sequence that is derived from a plant, bacteria, fungi, virus, or an animal. In some embodiments, the animal is a non-human animal, such as, by way of non-limiting example, a mouse, rat, hamster, rabbit, pig, bovine, deer, sheep, goat, chicken, cat, dog, ferret, a bird, non-human primate (e.g., marmoset, rhesus monkey). In some embodiments, the non-human animal is a domesticated mammal or an agricultural mammal. In some embodiments, the animal is a human. In some embodiments, the sequence comprises a human wild-type (WT) gene or a portion thereof. In some embodiments, the human WT gene or the portion thereof comprises a nucleotide sequence that is at least 70%, at least 80%, at least 90%, at least 92%, at least 95%, at least 97%, at least 99%, or 100% identical to an equal length portion of the WT sequence of any one of the sequences recited in any one of TABLE 9, 9.1, 9.2, and 9.3. In some embodiments, the donor nucleic acid is incorporated into an insertion site of a target nucleic acid.

In some embodiments, the donor nucleic acid comprises single-stranded DNA or linear double-stranded DNA. In some embodiments, the donor nucleic acid comprises a nucleotide sequence encoding a functional polypeptide and/or wherein the donor nucleic acid comprises a wildtype sequence. In some embodiments, the donor nucleic acid comprises a protein coding sequence, a gene, a gene fragment, an exon, an intron, an exon fragment, an intron fragment, a gene regulatory fragment, a gene regulatory region fragment, coding sequences thereof, or combinations thereof. In some embodiments, the donor nucleic acid comprises a naturally occurring sequence. In some embodiments, the naturally occurring sequence does not contain a mutation. In some embodiments, the naturally occurring sequence does not contain a mutation as described herein.

In some embodiments, the donor nucleic acid comprises a gene fragment, an exon fragment, an intron fragment, a gene regulatory region fragment, or combinations thereof. In some embodiments, the fragment is at least 10, at least 20, at least 30, at least 40, at least 50, at least 60, at least 70, or at least 80 contiguous nucleotides.

In reference to a viral vector, the term donor nucleic acid refers to a sequence of nucleotides that will be or has been introduced into a cell following transfection of the viral vector. The donor nucleic acid may be introduced into the cell by any mechanism of the transfecting viral vector, including, but not limited to, integration into the genome of the cell or introduction of an episomal plasmid or viral genome.

As another example, when used in reference to the activity of an effector protein, the term donor nucleic acid refers to a sequence of nucleotides that will be or has been inserted at the site of cleavage by the effector protein (cleaving (hydrolysis of a phosphodiester bond) of a nucleic acid resulting in a nick or double strand break-nuclease activity).

As yet another example, when used in reference to homologous recombination, the term donor nucleic acid refers to a sequence of DNA that serves as a template in the process of homologous recombination, which may carry the modification that is to be or has been introduced into the target nucleic acid. By using this donor nucleic acid as a template, the genetic information, including the modification, is copied into the target nucleic acid by way of homologous recombination.

Donor nucleic acids of any suitable size may be integrated into a target nucleic acid or genome. In some instances, the donor polynucleotide integrated into a genome is less than 3, about 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 16, 17, 18, 19, 20, 25, 30, 35, 40, 45, 50, 100, 150, 200, 250, 300, 350, 400, 450, 500 kilobases in length. In some instances, donor nucleic acids are more than 500 kilobases (kb) in length.

The donor nucleic acid may comprise a sequence that is derived from a plant, bacteria, virus or an animal. The animal may be human. The animal may be a non-human animal, such as, by way of non-limiting example, a mouse, rat, hamster, rabbit, pig, bovine, deer, sheep, goat, chicken, cat, dog, ferret, a bird, non-human primate (e.g., marmoset, rhesus monkey). The non-human animal may be a domesticated mammal or an agricultural mammal.

### Genetically Modified Cells and Organisms

Methods of editing described herein may be employed to generate a genetically modified cell. The cell may be a eukaryotic cell (e.g., a mammalian cell) or a prokaryotic cell (e.g., an archacal cell). The cell may be derived from a multicellular organism and cultured as a unicellular entity. The cell may comprise a heritable genetic modification, such that progeny cells derived therefrom comprise the heritable genetic mutation. The cell may be progeny of a genetically modified cell comprising a genetic modification of the genetically modified parent cell. A genetically modified cell may comprise a deletion, insertion, mutation, or non-native sequence relative to a wild-type version of the cell or the organism from which the cell was derived.

In some embodiments, upon modification of a target nucleic acid by compositions, systems, and methods described herein, the target nucleic acid can comprise an exon deletion, exon skipping, exon reframing, exon knock-in, or any combination thereof. In certain embodiments, cells and organism described herein can comprise a modified target nucleic acid comprising a splicing disruption, frameshift (e.g., 1+ or 2+ frameshift), sequence deletion, sequence skipping, sequence reframing, sequence knock-in, or any combination thereof, relative to a target nucleic acid that is not modified by the compositions, systems, or methods described herein.

Methods may comprise contacting a cell with a nucleic acid (e.g., a plasmid or mRNA) comprising a nucleobase sequence encoding an effector protein, wherein the effector protein comprises an amino acid sequence that is at least 70%, at least 75%, at least 80%, at least 85%, at least 90%, at least 95%, at least 97%, at least 98%, or at least 99% identical to any one of the sequences of TABLE 1.

Methods may comprise contacting cells with a nucleic acid (e.g., a plasmid or mRNA) comprising a nucleobase sequence encoding a guide nucleic acid, a tracrRNA, a crRNA, or any combination thereof. In some instances, the nucleobase sequence of the guide nucleic acid is at least 70%, at least 75%, at least 80%, at least 85%, at least 90%, at least 92%, at least 95%, at least 97%, at least 98%, at least 99%, or 100% identical to of any one of the gRNA sequences of TABLE 6. In some instances, the guide nucleic acid comprises a crRNA sequence comprising a spacer sequence that is at least 70%, at least 75%, at least 80%, at least 85%, at least 90%, at least 92%, at least 95%, at least 97%, at least 98%, at least 99%, or 100% identical to any one of the sequences of TABLE 4 and a repeat sequence that is at least 70%, at least 75%, at least 80%, at least 85%, at least 90%, at least 92%, at least 95%, at least 97%, at least 98%, at least 99%, or 100% identical to the sequence of TABLE 5. Contacting may comprise electroporation, acoustic poration, optoporation, viral vector-based delivery, iTOP, nanoparticle delivery (e.g., lipid or gold nanoparticle delivery), cell-penetrating peptide (CPP) delivery, DNA nanostructure delivery, or any combination thereof.

Methods may comprise contacting a cell with an effector protein or a multimeric complex thereof, wherein the effector protein comprises an amino acid sequence that is at least 70%, at least 75%, at least 80%, at least 85%, at least 90%, at least 95%, at least 97%, at least 98%, or at least 99% identical to any one of the sequences of TABLE 1.

Methods of the disclosure may be performed in a subject. Compositions of the disclosure may be administered to a subject. A subject may be a human. A subject may be a mammal (e.g., rat, mouse, cow, dog, pig, sheep, horse). A subject may be a vertebrate or an invertebrate. A subject may be a laboratory animal. A subject may be a patient. A subject may be at risk of developing, suffering from, or displaying symptoms a disease or disorder as set forth in TABLE 10. A subject may be at risk of developing Duchenne muscular dystrophy. A subject may be suffering from Duchenne muscular dystrophy. A subject may display symptoms of Duchenne muscular dystrophy. The subject may have a mutation associated with the DMD) gene. The subject may display symptoms associated with a mutation of the DMI) gene. In some embodiments, a mutation comprises a point mutation or single nucleotide polymorphism (SNP), a chromosomal mutation, a copy number mutation, or any combination thereof. A point mutation optionally comprises a substitution, insertion, or deletion. In some embodiments, a mutation comprises a chromosomal mutation. A chromosomal mutation can comprise an inversion, a deletion, a duplication, or a translocation. In some embodiments, a mutation comprises a copy number variation. A copy number variation can comprise a gene amplification or an expanding trinucleotide repeat. In some embodiments, mutations may be as set forth in TABLE 9.4.

Symptoms of muscular dystrophy, including DMD, may vary from mild to severe and may depend on what part of the body is affected, the causative mutation, and the age and overall health of the affected person, can include, e.g., fatigue, learning difficulties, intellectual disability, muscle weakness (e.g., in the legs, pelvis, arms, neck, diaphragm, heart, or other areas of the body), difficulty with motor skills (e.g., running, hopping, or jumping), frequent falls, trouble getting up from a lying position or climbing stairs, progressive difficulty walking, breathing difficulties, heart disease, abnormal heart muscle (e.g., cardiomyopathy), congestive heart failure, irregular heart rhythm (e.g., arrhythmias), deformities of the chest or back (scoliosis), enlarged muscles of the calves, buttocks, or shoulders, pseudohypertrophy, muscle deformities, respiratory disorders (e.g., pneumonia or poor swallowing). Symptoms can be measured for example, by utilizing: electromyography (EMG), genetic tests, muscle biopsy, serum Creatine Kinase (CK) levels, muscular strength tests (e.g., manual muscle testing), or range-of-motion (ROM) tests such as the six minute walk test.

Methods of the disclosure may be performed in a cell. A cell may be in vitro. A cell may be in vivo. A cell may be ex vivo. A cell may be an isolated cell. A cell may be a cell inside of an organism. A cell may be an organism. A cell may be a cell in a cell culture. A cell may be one of a collection of cells. A cell may be a mammalian cell or derived from a mammalian cell. A cell may be a rodent cell or derived from a rodent cell. A cell may be a human cell or derived from a human cell. A cell may be a eukaryotic cell or derived from a eukaryotic cell. A cell may be a pluripotent stem cell. A cell may be a plant cell or derived from a plant cell. A cell may be an animal cell or derived from an animal cell. A cell may be an invertebrate cell or derived from an invertebrate cell. A cell may be a vertebrate cell or derived from a vertebrate cell.

A cell may be from a specific organ or tissue. In some embodiments, the cell is a hepatocyte. In some embodiments, the tissue is a subject's blood, bone marrow, or cord blood. In some embodiments, the tissue is a heterologous donor blood, cord blood, or bone marrow. In some embodiments, the tissue is an allogenic blood, cord blood, or bone marrow.

The tissue may be muscle. The muscle may be skeletal muscle. In certain instances, skeletal muscles include the following: abductor digiti minimi (foot), abductor digiti minimi (hand), abductor hallucis, abductor pollicis brevis, abductor pollicis longus, adductor brevis, adductor hallucis, adductor longus, adductor magnus, adductor pollicis, anconeus, articularis cubiti, articularis genu, aryepiglotticus, auricularis, biceps brachii, biceps femoris, brachialis, brachioradialis, buccinator, bulbospongiosus, constrictor of pharynx-inferior, constrictor of pharynx-middle, constrictor of pharynx-superior, coracobrachialis, corrugator supercilii, cremaster, cricothyroid, dartos, decp transverse perinei, deltoid, depressor anguli oris, depressor labii inferioris, diaphragm, digastric, digastric (anterior view), erector spinae-spinalis, erector spinae-iliocostalis, erector spinae-longissimus, extensor carpi radialis brevis, extensor carpi radialis longus, extensor carpi ulnaris, extensor digiti minimi (hand), extensor digitorum (hand), extensor digitorum brevis (foot), extensor digitorum longus (foot), extensor hallucis brevis, extensor hallucis longus, extensor indicis, extensor pollicis brevis, extensor pollicis longus, external oblique abdominis, flexor carpi radialis, flexor carpi ulnaris, flexor digiti minimi brevis (foot), flexor digiti minimi brevis (hand), flexor digitorum brevis, flexor digitorum longus (foot), flexor digitorum profundus, flexor digitorum superficialis, flexor hallucis brevis, flexor hallucis longus, flexor pollicis brevis, flexor pollicis longus, frontalis, gastrocnemius, gemellus inferior, gemellus superior, genioglossus, geniohyoid, gluteus maximus, gluteus medius, gluteus minimus, gracilis, hyoglossus, iliacus, inferior oblique, inferior rectus, infraspinatus, intercostals external, intercostals innermost, intercostals internal, internal oblique abdominis, interossei-dorsal of hand, interossei-dorsal of foot, interossei-palmar of hand, interossei plantar of foot, interspinales, intertransversarii, intrinsic muscles of tongue, ishiocavernosus, lateral cricoarytenoid, lateral pterygoid, lateral rectus, latissimus dorsi, levator anguli oris, levator ani-coccygeus, levator ani-iliococcygeus, levator ani-pubococcygeus, levator ani-puborectalis, levator ani-pubovaginalis, levator labii superioris, levator labii superioris, alacque nasi, levator palpebrac superioris, levator scapulae, levator veli palatini, levatores costarum, longus capitis, longus colli, lumbricals of foot, lumbricals of hand, masseter, medial pterygoid, medial rectus, mentalis, m, uvulac, mylohyoid, nasalis, oblique arytenoid, obliquus capitis inferior, obliquus capitis superior, obturator externus, obturator internus (A), obturator internus (B), omohyoid, opponens digiti minimi (hand), opponens pollicis, orbicularis oculi, orbicularis oris, palatoglossus, palatopharyngeus, palmaris brevis, palmaris longus, pectineus, pectoralis major, pectoralis minor, peroneus brevis, peroneus longus, peroneus tertius, piriformis (A), piriformis (B), plantaris, platysma, popliteus, posterior cricoarytenoid, procerus, pronator quadratus, pronator teres, psoas major, psoas minor, pyramidalis, quadratus femoris, quadratus lumborum, quadratus plantac, rectus abdominis, rectus capitus anterior, rectus capitus lateralis, rectus capitus posterior major, rectus capitus posterior minor, rectus femoris, rhomboid major, rhomboid minor, risorius, salpingopharyngeus, sartorius, scalenus anterior, scalenus medius, scalenus minimus, scalenus posterior, semimembranosus, semitendinosus, serratus anterior, serratus posterior inferior, serratus posterior superior, soleus, sphincter ani, sphincter urethrac, splenius capitis, splenius cervicis, stapedius, sternocleidomastoideornohyoid, sternothyroid, styloglossus, stylohyoid, stylohyoid (anterior view), stylopharyngeus, subclavius, subcostalis, subscapularis, superficial transverse perinei, superior oblique, superior rectus, supinator, supraspinatus, temporalis, temporoparietalis, tensor fasciae lata, tensor tympani, tensor veli palatini, teres major, teres minor, thyro-arytenoid & vocalis, thyro-epiglotticus, thyrohyoid, tibialis anterior, tibialis posterior, transverse arytenoid, transversospinalis-multifidus, transversospinalis-rotatores, transversospinalis-semispinalis, transversus abdominis, transversus thoracis, trapezius, triceps, vastus intermedius, vastus lateralis, vastus medialis, zygomaticus major, or zygomaticus minor. In some instances, the cell is a myocyte. In some instances, the cell is a muscle cell. In some instances, the muscle cell is a skeletal muscle cell. In some instances, the skeletal muscle cell is a red (slow) skeletal muscle cell, a white (fast) skeletal muscle cell or an intermediate skeletal muscle cell.

The tissue may be the subject's blood, bone marrow, or cord blood. The tissue may be heterologous donor blood, cord blood, or bone marrow. The tissue may be allogenic blood, cord blood, or bone marrow. In some instances, the cell is a: a stem cell, muscle satellite cell, muscle stem cell, myoblast, muscle progenitor cell, a pluripotent stem cell or a cell derived from a pluripotent stem cell.

Methods of editing described herein may comprise contacting cells with compositions or systems described herein. In some embodiments, the contacting comprises electroporation, acoustic poration, optoporation, viral vector-based delivery, iTOP, nanoparticle delivery (e.g., lipid or gold nanoparticle delivery), cell-penetrating peptide (CPP) delivery. DNA nanostructure delivery, or any combination thereof.

Methods of editing described herein may be performed in a subject. In some embodiments, the methods comprise administering compositions described herein to the subject. In some embodiments, the subject is a human. In some embodiments, the subject is a mammal (e.g., rat, mouse, cow, dog, pig, sheep, horse). In some embodiments, the subject is a vertebrate or an invertebrate. In some embodiments, the subject is a laboratory animal. In some embodiments, the subject is a patient. In some embodiments, the subject is at risk of developing, suffering from, or displaying symptoms of a disease, for example set forth in TABLE 10. In some embodiments, the subject may have a mutation associated with a gene described herein. In some embodiments, the subject may display symptoms associated with a mutation of a gene described herein, for example set forth in TABLE 9.

### XI. Method of Treating a Disorder

Described herein are methods for treating a disease in a subject by modifying, such as editing, a target nucleic acid associated with a gene or expression of a gene related to the disease. In some embodiments, the disease or disorder comprises one or more of the diseases or disorder set forth in TABLE 10.

In some embodiments, the method for treating a disease comprises modifying at least one gene associated with the disease or modifying expression of the at least one gene such that the disease is treated. In some embodiments, the disease is any one of the diseases or disorders set forth in TABLE 10 and the gene is the gene set forth in TABLE 9. In some embodiments, the disease is Duchenne Muscular Dystrophy and the gene is DMI).

Modifying at least one gene using the compositions and methods described herein can, in some embodiments, induce a reduction or increase in expression of the one or more genes. In some embodiments, the at least one modified gene results in a reduction in expression, also referred to as gene silencing. In some embodiments, the gene silencing reduces expression of one or more genes by at least 10%, at least 20%, at least 30%, at least 40%, at least 50%, at least 60%, at least 70%, at least 80%, at least 90%, or at least 95%. In some embodiments, gene silencing is accomplished by transcriptional silencing, post-transcriptional silencing, or meiotic silencing. In some embodiments, transcriptional silencing is by genomic imprinting, paramutation, transposon silencing, position effect, or RNA-directed DNA methylation. In some embodiments, post-transcriptional silencing is by RNA interference. RNA silencing, or nonsense mediated decay. In some embodiments, meiotic silencing is by transfection or meiotic silencing of unpaired DNA. In some embodiments, the at least one modified gene results in removing all expression, also referred to as the gene being knocked out (KO).

In some embodiments, a gene is modified by repairing or editing a mutation as described herein. In some cases, an effector protein (e.g., Cas protein) is used to effect the modification. Cas proteins may be fused to transcription activators or transcriptional repressors or deaminases or other nucleic acid modifying proteins. In some cases, effector proteins (e.g., Cas proteins) need not be fused to a partner protein to accomplish the required protein (expression) modification.

In some embodiments, methods for treating a disease in a subject comprises administration of a composition(s) or component(s) of a system described herein. In some embodiments, the composition(s) or component(s) of the system comprises use of a recombinant nucleic acid (DNA or RNA), administered for the purpose to edit a nucleic acid. In some embodiments, the composition or component of the system comprises use of a vector to introduce a functional gene or transgene. In some embodiments, treatment of a disease comprises administration of a gene therapy. “Gene therapy”, as used herein, comprises use of a recombinant nucleic acid (DNA or RNA), administered for the purpose to adjust, repair, replace, add, or remove a gene sequence. In some embodiments, a gene therapy comprises use of a vector to introduce a functional gene or transgene. In some embodiments, vectors comprise nonviral vectors, including cationic polymers, cationic lipids, or bio-responsive polymers. In some embodiments, the bio-responsive polymer exploits chemical-physical properties of the endosomal environment (e.g., pH) to preferentially release the genetic material in the intracellular space. In some embodiments, vectors comprise viral vectors, including retroviruses, adenoviruses, adeno-associated viruses, and herpes simplex viruses. In some embodiments, the vector comprises a replication-defective viral vector, comprising an insertion of a therapeutic gene inserted in genes essential to the lytic cycle, preventing the virus from replicating and exerting cytotoxic effects. Any suitable method of gene therapy may be applicable to the compositions and systems described herein. Methods of gene therapy are described in more detail in Ingusci et al., “Gene Therapy Tools for Brain Diseases”, Front. Pharmacol. 10:724 (2019) which is hereby incorporated by reference in its entirety.

In some embodiments, a genome targeted for treatment comprises a wild-type DMD gene or a mutated DMD gene. In some embodiments, the genome comprises a mutated DMD target gene.

In some embodiments, treating, preventing, or inhibiting disease or disorder in a subject may comprise contacting a target nucleic acid associated with a particular ailment with a composition described herein. In some aspects, the methods of treating, preventing, or inhibiting a disease or disorder may involve removing, modifying, replacing, transposing, or affecting the regulation of a genomic sequence of a patient in need thereof. In some embodiments, the methods of treating, preventing, or inhibiting a disease or disorder may involve modulating gene expression.

Described herein are compositions and methods for treating a disease in a subject by editing a target nucleic acid associated with a gene or expression of a gene related to the disease. In some embodiments, methods comprise administering a composition or cell described herein to a subject. By way of non-limiting example, the disease may be a cancer, an ophthalmological disorder, a neurological disorder, a neurodegenerative disease, a blood disorder, or a metabolic disorder, or a combination thereof. The disease may be an inherited disorder, also referred to as a genetic disorder. The disease may be the result of an infection or associated with an infection. Also, by way of non-limiting example, the compositions are pharmaceutical compositions described herein.

The compositions and methods described herein may be used to treat, prevent, or inhibit a disease or syndrome in a subject. In some embodiments, the disease is a liver disease, a lung disease, an eye disease, or a muscle disease. Exemplary diseases and syndromes include but are not limited to the diseases and syndromes listed in TABLE 10.

### Sequences and Tables

TABLE 1 provides illustrative amino acid sequences of effector proteins that are useful in the compositions, systems and methods described herein.

TABLE 1
EXEMPLARY AMINO ACID SEQUENCE(S) OF EFFECTOR PROTEIN(S)

| SEQ | | |
|---|---|---|
| ID | | |
| NO: | ID | Sequence |
| 1 | CasPhi.32 | VPDKKETPLVALCKKSFPGLRFKKHDSRQAGRILKSKGEGAAVAFLEGKGGTTQPNFKPPVKCNIVAMSRPLEE |
| | | WPIYKASVVIQKYVYAQSYEEFKATDPGKSEAGLRAWLKATRVDTDGYFNVQGLNLIFQNARATYEGVLKKVE |
| | | NRNSKKVAKIEQRNEHRAERGLPLLTLDEPETALDETGHLRHRPGINCSVFGYQHMKLKPYVPGSIPGVTGYSRD |
| | | PSTPIAACGVDRLEIPEGQPGYVPPWDRENLSVKKHRRKRASWARSRGGAIDDNMLLAVVRVADDWALLDLRG |
| | | LLRNTQYRKLLDRSVPVTIESLLNLVINDPTLSVVKKPGKPVRYTATLIYKQGVVPVVKAKVVKGSYVSKMLDD |
| | | TTETFSLVGVDLGVNNLIAANALRIRPGKCVERLQAFTLPEQTVEDFFRFRKAYDKHQENLRLAAVRSLTAEQQA |
| | | EVLALDTFGPEQAKMQVCGHLGLSVDEVPWDKVNSRSSILSDLAKERGVDDTLYMFPFFKGKGKKRKTEIRKR |
| | | WDVNWAQHFRPQLTSETRKALNEAKWEAERNSSKYHQLSIRKKELSRHCVNYVIRTAEKRAQCGKVIVAVEDL |
| | | HHSFRRGGKGSRKSGWGGFFAAKQEGRWLMDALFGAFCDLAVHRGYRVIKVDPYNTSRTCPECGHCDKANRD |
| | | RVNREAFICVCCGYRGNADIDVAAYNIAMVAITGVSLRKAARASVASTPLESLAAE |
| 2 | CasPhi.12 | MIKPTVSQFLTPGFKLIRNHSRTAGLKLKNEGEEACKKFVRENEIPKDECPNFQGGPAIANIIAKSREFTEWEIYQS |
| | | SLAIQEVIFTLPKDKLPEPILKEEWRAQWLSEHGLDTVPYKEAAGLNLIIKNAVNTYKGVQVKVDNKNKNNLAKIN |
| | | RKNEIAKLNGEQEISFEEIKAFDDKGYLLQKPSPNKSIYCYQSVSPKPFITSKYHNVNLPEEYIGYYRKSNEPIVSP |
| | | YQFDRLRIPIGEPGYVPKWQYTFLSKKENKRRKLSKRIKNVSPILGIICIKKDWCVFDMRGLLRTNHWKKYHKPTDS |
| | | INDLFDYFTGDPVIDTKANVVRFRYKMENGIVNYKPVREKKGKELLENICDQNGSCKLATVDVGQNNPVAIGLFE |
| | | LKKVNGELTKTLISRHPTPIDFCNKITAYRERYDKLESSIKLDAIKQLTSEQKIEVDNYNNNFTPQNTKQIVCSKLN |
| | | INPNDLPWDKMISGTHFISEKAQVSNKSEIYFTSTDKGKTKDVMKSDYKWFQDYKPKLSKEVRDALSDIEWRLRR |
| | | ESLEFNKLSKSREQDARQLANWISSMCDVIGIENLVKKNNFFGGSGKREPGWDNFYKPKKENRWWINAIHKALT |
| | | ELSQNKGKRVILLPAMRTSITCPKCKYCDSKNRNGEKFNCLKCGIELNADIDVATENLATVAITAQSMPKPTCERS |
| | | GDAKKPVRARKAKAPEFHDKLAPSYTVVLREAV |
| 3 | CasM. | MPTITRKIELTLLTEGLSEEQRKEQWGLLYHINDNLYKAANNISSKLYLDDHVSSMVRMKHAEYLSLLKELARAE |
| | 19952 | KQKTPDADAIAELRKKVAAAEKEMTDQEHAICKYATEMSTQSLSYRFATELETNIFAKILDCLKQGVFATFNSDA |
| | | RDVKRGERAIRNYKKGMPIPFAWDKSLRIEKDNKDFYLRWYNGLRFLFNFGKDRSNNRLIVERCLKMDADYDG |
| | | EYKLCNSSIQIAKREGKTKLFLLLVVKIPQEHVELNKKVVVGVDLGINVPAYVATNITEERKAIGDREHFLNSRMA |
| | | FQRRYKSLQRLRGTAGGKGRAKKLEPLERLRKAEHNWVHTQNHLFSREVVDFAVKSHAATIHMEDLSGFGKDN |
| | | DGNADERKEFVLRNWSYYELQNMIAYKAAKYGIKVEKIHPAYTSKTCSWCGQLGFREGVTFICENPECKQCGEK |
| | | VHADYNAARNIANSKDIIKKNE |
| 4 | Cas14a.1 | MAKNTITKTLKLRIVRPYNSAEVEKIVADEKNNREKIALEKNKDKVKEACSKHLKVAAYCTTQVERNACLFCKA |
| | | RKLDDKFYQKLRGQFPDAVFWQEISEIFRQLQKQAAEIYNQSLIELYYEIFIKGKGIANASSVEHYLSDVCYTRAA |
| | | ELFKNAAIASGLRSKIKSNFRLKELKNMKSGLPTTKSDNFPIPLVKQKGGQYTGFEISNHNSDFIIKIPFGRWQVKK |
| | | EIDKYRPWEKFDFEQVQKSPKPISLLLSTQRRKRNKGWSKDEGTEAEIKKVMNGDYQTSYIEVKRGSKIGEKSAW |
| | | MLNLSIDVPKIDKGVDPSIIGGIDVGVKSPLVCAINNAFSRYSISDNDLFHFNKKMFARRRILLKKNRHKRAGHGA |
| | | KNKLKPITILTEKSERFRKKLIERWACEIADFFIKNKVGTVQMENLESMKRKEDSYFNIRLRGFWPYAEMQNKIEF |
| | | KLKQYGIEIRKVAPNNTSKTCSKCGHLNNYFNFEYRKKNKFPHFKCEKCNFKENADYNAALNISNPKLKSTKEEP |

TABLE 1.1 provides exemplary amino acid alterations relative to SEQ ID NO: 2 useful in compositions, systems, and methods described herein.

TABLE 1.1
EXEMPLARY AMINO ACID ALTERATIONS
RELATIVE TO SEQ ID NO: 2

| Effector Protein SEQ ID | |
|---|---|
| NO: | Amino Acid Alterations |
| 2 | L26R, L26H, L26K |

TABLE 2 provides illustrative sequences of exemplary heterologous polypeptide modifications of effector protein(s) that are useful in the compositions, systems and methods described herein.

TABLE 2
SEQUENCES OF EXEMPLARY HETEROLOGOUS POLYPEPTIDE MODIFICATIONS OF EFFECTOR
PROTEIN(S)

| SEQ ID NO: | Description | Sequence |
|---|---|---|
| 5 | NLS | KR(K/R)R |
| 6 | NLS | (P/R)XXKR(^{∧}D/E)(K/R) |
| 7 | NLS | KRX(W/F/Y)XXAF |
| 8 | NLS | (R/P)XXKR(K/R)(^{∧}D/E) |
| 9 | NLS | LGKR(K/R)(W/F/Y) |
| 10 | NLS | KRX₁₀K(K/R)(K/R) |
| 11 | EEP | GLFXALLXLLXSLWXLLLXA |
| 12 | NLS | K(K/R)RK |
| 13 | NLS | KRX₁₁K(K/R)(K/R) |
| 14 | NLS | KRX₁₂K(K/R)(K/R) |
| 15 | NLS | KRX₁₀K(K/R)X(K/R) |
| 16 | NLS | KRX₁₁K(K/R)X(K/R) |
| 17 | NLS | KRX₁₂K(K/R)X(K/R) |
| 18 | NLS | APKKKRKVGIHGVPAA |
| 19 | EEP | GLFX*ALLX*LLX*SLWX*LLLX*A |
| 20 | EEP | GLFHALLHLLHSLWHLLLHA |
| 21 | NLS | PKKKRKVGIHGVPAA |
| 22 | Nucleoplasmin NLS | KRPAATKKAGQAKKKK |
| 23 | FLAG tag | MDYKDHDGDYKDHDIDYKDDDDK |
| 24 | NLS | MAPKKKRKV |
| 25 | Linker | GIHGVPAA |
| 26 | Linker + T2A | EFGSGEGRGSLLTCGDVEENPGP |
| | Sequence | |
| 27 | Blastin Resistance | MAKPLSQEESTLIERATATINSIPISEDYSVASAALSSDGRIFTGVNVYHFTGG |
| | | PCAELVVLGTAAAAAAGNLTCIVAIGNENRGILSPCGRCRQVLLDLHPGIKAIV |
| | | KDSDGQPTAVGIRELLPSGYVWEG |

*wherein X is any naturally occurring amino acid; histidine, or arginine.D/E is any naturally occurring amino acid except Asp or Glu; and wherein X* is any one of lysine, histidine, or arginine.

TABLE 2.1 provides illustrative sequences of exemplary effector protein(s) modified with heterologous polypeptides that are useful in the compositions, systems and methods described herein.

TABLE 2.1
SEQUENCES OF EFFECTOR PROTEIN(S) WITH EXEMPLARY HETEROLOGOUS POLYPEPTIDE
MODIFICATIONS

| SEQ ID | Modification | |
|---|---|---|
| NO: | Description | Sequence |
| 1468 | NLS, SEQ | PKKKRKVGIHGVPAAMIKPTVSQFLTPGFKLIRNHSRTAGLKLKNEGEEACKKFVRENEIPKDECPNF |
| | ID NO: 2, | QGGPAIANIIAKSREFTEWEIYQSSLAIQEVIFTLPKDKLPEPILKEEWRAQWLSEHGLDTVPYKEAAG |
| | NLS | LNLIIKNAVNTYKGVQVKVDNKNKNNLAKINRKNEIAKLNGEQEISFEEIKAFDDKGYLLQKPSPNKS |
| | | IYCYQSVSPKPFITSKYHNVNLPEEYIGYYRKSNEPIVSPYQFDRLRIPIGEPGYVPKWQYTFLSKKEN |
| | | KRRKLSKRIKNVSPILGIICIKKDWCVFDMRGLLRTNHWKKYHKPTDSINDLFDYFTGDPVIDTKANV |
| | | VRFRYKMENGIVNYKPVREKKGKELLENICDQNGSCKLATVDVGQNNPVAIGLFELKKVNGELTKT |
| | | LISRHPTPIDFCNKITAYRERYDKLESSIKLDAIKQLTSEQKIEVDNYNNNFTPQNTKQIVCSKLNINPN |
| | | DLPWDKMISGTHFISEKAQVSNKSEIYFTSTDKGKTKDVMKSDYKWFQDYKPKLSKEVRDALSDIEW |
| | | RLRRESLEFNKLSKSREQDARQLANWISSMCDVIGIENLVKKNNFFGGSGKREPGWDNFYKPKKENR |
| | | WWINAIHKALTELSQNKGKRVILLPAMRTSITCPKCKYCDSKNRNGEKFNCLKCGIELNADIDVATE |
| | | NLATVAITAQSMPKPTCERSGDAKKPVRARKAKAPEFHDKLAPSYTVVLREAVKRPAATKKAGQAKKK |
| | | K |
| 1469 | FLAG TAG, | MDYKDHDGDYKDHDIDYKDDDDKMAPKKKRKVGIHGVPAAMIKPTVSQFLTPGFKLIRNHSRTAGLKLK |
| | NLS, Linker, | NEGEEACKKFVRENEIPKDECPNFQGGPAIANIIAKSREFTEWEIYQSSLAIQEVIFTLPKDKLPEPILKE |
| | SEQ ID NO: | EWRAQWLSEHGLDTVPYKEAAGLNLIIKNAVNTYKGVQVKVDNKNKNNLAKINRKNEIAKLNGEQEISFEEI |
| | 2, NLS, | KAFDDKGYLLQKPSPNKSIYCYQSVSPKPFITSKYHNVNLPEEYIGYYRKSNEPIVSPYQFDRLRIPIGEPG |
| | Linker + T2A | YVPKWQYTFLSKKENKRRKLSKRIKNVSPILGIICIKKDWCVFDMRGLLRTNHWKKYHKPTDSINDLFDYFT |
| | sequence, | GDPVIDTKANVVRFRYKMENGIVNYKPVREKKGKELLENICDQNGSCKLATVDVGQNNPVAIGLFELKKVNG |
| | Blasticidin | ELTKTLISRHPTPIDFCNKITAYRERYDKLESSIKLDAIKQLTSEQKIEVDNYNNNFTPQNTKQIVCSKLNI |
| | reistance | NPNDLPWDKMISGTHFISEKAQVSNKSEIYFTSTDKGKTKDVMKSDYKWFQDYKPKLSKEVRDALSDIEWRL |
| | | RRESLEFNKLSKSREQDARQLANWISSMCDVIGIENLVKKNNFFGGSGKREPGWDNFYKPKKENRWWINA |
| | | IHKALTELSQNKGKRVILLPAMRTSITCPKCKYCDSKNRNGEKFNCLKCGIELNADIDVATENLATVAITAQ |
| | | SMPKPTCERSGDAKKPVRARKAKAPEFHDKLAPSYTVVLREAVKRPAATKKAGQAKKKKEFGSGEGRG |
| | | SLLTCGDVEENPGPMAKPLSQEESTLIERATATINSIPISEDYSVASAALSSDGRIFTGVNVYHFTGGPCAE |
| | | LVVLGTAAAAAAGNLTCIVAIGNENRGILSPCGRCRQVLLDLHPGIKAIVKDSDGQPTAVGIRELLPSGYVW |
| | | EG |

TABLE 3 provides illustrative PAM sequences that are useful in the compositions, systems and methods described herein.

TABLE 3
EXEMPLARY PAM SEQUENCES

| Effector Protein | PAM Sequence* |
|---|---|
| SEQ ID NO: | (5′→3′) |
| 1 | GTTA |
| 1 | GTTT |
| 1 | GTTC |
| 1 | GTTG |
| 2 | TTTA |
| 2 | TTTT |
| 2 | CTTT |
| 2 | ATTA |
| 2 | ATTT |
| 2 | CTTG |
| 2 | TTTG |
| 2 | GTTT |
| 2 | GTTA |
| 2 | TTTC |
| 2 | CTTC |
| 2 | CTTA |
| 2 | GTTC |
| 2 | ATTG |
| 2 | GTTG |
| 2 | ATTC |
| 2 | TTTA |
| 2 | NTTN |
| 3 | TTCG |
| 3 | ATCG |
| 3 | GTCG |
| 4 | TTTA |
| 4 | TTTG |

TABLE 4 provides illustrative spacer sequences for use in guide nucleic acids that are useful in the compositions, systems and methods described herein.

TABLE 4
EXEMPLARY SPACER SEQUENCES FOR USE IN GUIDE NUCLEIC ACIDS

| | Effector | | Spacer |
|---|---|---|---|
| Composition | Protein | Spacer Sequence (5′→3′), | Sequence |
| Name | SEQ ID NO: | shown as RNA | SEQ ID NO: |
| T01_CasPhi32 | 1 | UACUGACAAAGAUAUCACUC | 28 |
| T02_CasPhi32 | 1 | CCUGCAUUUGCAGAGUUUAG | 29 |
| T03_CasPhi32 | 1 | CUUGAAACUAAACUCUGCAA | 30 |
| T04_CasPhi32 | 1 | UACAUAAUCCAUCUAUUUUU | 31 |
| T05_CasPhi32 | 1 | UGAAGGGUGAUGGAAAUUAC | 32 |
| T06_CasPhi32 | 1 | AAGAGUCCAGAUGUGCUGAA | 33 |
| T07_CasPhi32 | 1 | UCAUCAUUAUAUUACUAGAA | 34 |
| T08_CasPhi32 | 1 | UUUCGAAAUUGUAUUUAUCU | 35 |
| T09_CasPhi32 | 1 | UUGUCAUCAUUAUAUUACUA | 36 |
| T10_CasPhi32 | 1 | CAUAGGGAAAUUUUCACAUG | 37 |
| T11_CasPhi32 | 1 | CUGUAAGAUACCAAAAAGGC | 38 |
| T12_CasPhi32 | 1 | UGCCUUUUUGGUAUCUUACA | 39 |
| T13_CasPhi32 | 1 | UACUGGCAAAGAAAGAAAUA | 40 |
| T14_CasPhi32 | 1 | AAUCUUUUCUCAAAUAAAAA | 41 |
| T15_CasPhi32 | 1 | AUUAAUGGUUGAUAGGUUCU | 42 |
| T16_CasPhi32 | 1 | GUGCCUUUCACCCUGCUUAU | 43 |
| T17_CasPhi32 | 1 | UCAUUCCUAUUAGAUCUGUC | 44 |
| T18_CasPhi32 | 1 | AUAGGUUCUUUAAUGUUAGU | 45 |
| T19_CasPhi32 | 1 | UUUAAUGUUAGUGCCUUUCA | 46 |
| T20_CasPhi32 | 1 | AUCGAAUAAGUAAUGUGUAU | 47 |
| T21_CasPhi32 | 1 | GAAGAUCUGAGCUCUGAGUG | 48 |
| T22_CasPhi32 | 1 | AAGAGGAAGUUAGAAGAUCU | 49 |
| T23_CasPhi32 | 1 | AUGAAUUAUCUUCAAAGUGU | 50 |
| T24_CasPhi32 | 1 | AUUUAGAAGUAAAAUAUAAU | 51 |
| T25_CasPhi32 | 1 | AAAAGUGCAACUAUGAAGUG | 52 |
| T26_CasPhi32 | 1 | AUCUCUAUCUUUAGAAUUGA | 53 |
| T27_CasPhi32 | 1 | CAAUUCUAAAGAUAGAGAUA | 54 |
| T28_CasPhi32 | 1 | CACUUUUGAACAAAUAGCUA | 55 |
| T29_CasPhi32 | 1 | UUGACUGUGCAAAAAGUCUU | 56 |
| T30_CasPhi32 | 1 | GGCUCAAAUUGUUACUCUUC | 57 |
| T31_CasPhi32 | 1 | UUGAAAUUGGCUCUUUAGCU | 58 |
| T32_CasPhi32 | 1 | CUCUUCAAUUAAAUUUGACU | 59 |
| T33_CasPhi32 | 1 | UUGCAAAAAGGAAAAAAGAA | 60 |
| T34_CasPhi32 | 1 | CUAAUUUUUCUUUUUCUUCU | 61 |
| T35_CasPhi32 | 1 | GCUGAGAGAGAAACAGUUGC | 62 |
| T36_CasPhi32 | 1 | CUCUCUCAGCAAACACAUUA | 63 |
| T37_CasPhi32 | 1 | CCUAAGAACUGGUGGGAAAU | 64 |
| T38_CasPhi32 | 1 | UUAGGCAACUGUUUCUCUCU | 65 |
| T39_CasPhi32 | 1 | GCAGAAGUUUUUCUUUAAAA | 66 |
| T40_CasPhi32 | 1 | UUCUUUAAAAUGAAGAUUUU | 67 |
| T41_CasPhi32 | 1 | AUCAUCCUAGCCAUAACACA | 68 |
| T42_CasPhi32 | 1 | UUCAUCAUCCUAGCCAUAAC | 69 |
| T43_CasPhi32 | 1 | AAAGAAUUCAGAAUCAGUGG | 70 |
| T44_CasPhi32 | 1 | GGAAGUUACAUUUAAAAUGU | 71 |
| T45_CasPhi32 | 1 | CAUUUAAAAUGUCUCCUCCA | 72 |
| T46_CasPhi32 | 1 | UCUCACAUUUAUGUUGCUUA | 73 |
| T47_CasPhi32 | 1 | CUUAUUUAAAAAAUUAUUCA | 74 |
| T48_CasPhi32 | 1 | UUAACAGAAAAUCACGUUAA | 75 |
| T49_CasPhi32 | 1 | AAGCUGAAAUGAACAGUAGA | 76 |
| T50_CasPhi32 | 1 | AUUUCAGCUUUAACGUGAUU | 77 |
| T51_CasPhi32 | 1 | AUAACUUUACAUUAAACAUC | 78 |
| T52_CasPhi32 | 1 | GUAUCAAAGAUACCUUUUUA | 79 |
| T53_CasPhi32 | 1 | CAUUUGAUAAAAUUAUACCA | 80 |
| T54_CasPhi32 | 1 | AAUGUAAAGUUAUUAACAGA | 81 |
| T55_CasPhi32 | 1 | CUGUGAUUUUCUUUUGGAUU | 82 |
| T56_CasPhi32 | 1 | AAUCCUUUAACAUUUCAUUC | 83 |
| T57_CasPhi32 | 1 | UGAAGGUGUUCUUGUACUUC | 84 |
| T58_CasPhi32 | 1 | UUGUACUUCAUCCCACUGAU | 85 |
| T59_CasPhi32 | 1 | CCUCCGGUUCUGAAGGUGUU | 86 |
| T60_CasPhi32 | 1 | AAGGAUUCAACACAAUGGCU | 87 |
| T61_CasPhi32 | 1 | AAUGAAAUGUUAAAGGAUUC | 88 |
| T62_CasPhi32 | 1 | UGUCACCAGAGUAACAGUCU | 89 |
| T63_CasPhi32 | 1 | UGUCCAAGCCCGGUUGAAAU | 90 |
| T64_CasPhi32 | 1 | GGAGAUGGCAGUUUCCUUAG | 91 |
| T65_CasPhi32 | 1 | CCUUAGUAACCACAGGUUGU | 92 |
| T66_CasPhi32 | 1 | AAAUCUGCCAGAGCAGGUAC | 93 |
| T67_CasPhi32 | 1 | AUAUCCUCAAGGUCACCCAC | 94 |
| T68_CasPhi32 | 1 | UAAAAUCACAGAGGGUGAUG | 95 |
| T69_CasPhi32 | 1 | GAGGUACCUGCUCUGGCAGA | 96 |
| T70_CasPhi32 | 1 | CUCUGGUGACACAACCUGUG | 97 |
| T71_CasPhi32 | 1 | CUAAGGAAACUGCCAUCUCC | 98 |
| T72_CasPhi32 | 1 | ACCGCCUUCCACUCAGAGCU | 99 |
| T73_CasPhi32 | 1 | ACUUCAAGAGCUGAGGGCAA | 100 |
| T74_CasPhi32 | 1 | UUGAGGAUUGCUGAAUUAUU | 101 |
| T75_CasPhi32 | 1 | GCAGACCUCCUGCCACCGCA | 102 |
| T76_CasPhi32 | 1 | CAUUCAAUGUUCUGACAACA | 103 |
| T77_CasPhi32 | 1 | UGACAACAGUUUGCCGCUGC | 104 |
| T78_CasPhi32 | 1 | GCCGCUGCCCAAUGCCAUCC | 105 |
| T79_CasPhi32 | 1 | UCAGAACAUUGAAUGCAACU | 106 |
| T80_CasPhi32 | 1 | CCAAUUCUCAGGAAUUUGUG | 107 |
| T81_CasPhi32 | 1 | AGCUUCUGUUAGCCACUGAU | 108 |
| T82_CasPhi32 | 1 | GCCACUGAUUAAAUAUCUUU | 109 |
| T83_CasPhi32 | 1 | CUCAGAAAGACACAAAUUCC | 110 |
| T84_CasPhi32 | 1 | AGAAAUGGCGGCGUUUUCAU | 111 |
| T001_CasPhi.12 | 2 | CCUGCAGGCGAUUUGAC | 112 |
| T002_CasPhi.12 | 2 | ACCUGCAGGCGAUUUGA | 113 |
| T003_CasPhi.12 | 2 | UACCUGCAGGCGAUUUG | 114 |
| T004_CasPhi.12 | 2 | UAGAGAUAUAGCGUAUA | 115 |
| T005_CasPhi.12 | 2 | GGUUAUACUGACAAAGA | 116 |
| T006_CasPhi.12 | 2 | GACAGAUCUGUUGAGAA | 117 |
| T007_CasPhi.12 | 2 | CUCAACAGAUCUGUCAA | 118 |
| T008_CasPhi.12 | 2 | AUCCAUAUGCUUUUACC | 119 |
| T009_CasPhi.12 | 2 | GUUACUUGAAACUAAAC | 120 |
| T010_CasPhi.12 | 2 | GCAGAGUUUAGUUUCAA | 121 |
| T011_CasPhi.12 | 2 | GUUAUACUGACAAAGAU | 122 |
| T012_CasPhi.12 | 2 | UUUGGUUAUACUGACAA | 123 |
| T013_CasPhi.12 | 2 | CCUGCAUUUGCAGAGUU | 124 |
| T014_CasPhi.12 | 2 | CUUGAAACUAAACUCUG | 125 |
| T015_CasPhi.12 | 2 | UUCUUGAUCCAUAUGCU | 126 |
| T016_CasPhi.12 | 2 | CUUGAUCCAUAUGCUUU | 127 |
| T017_CasPhi.12 | 2 | UCUUGAUCCAUAUGCUU | 128 |
| T018_CasPhi.12 | 2 | UACUGACAAAGAUAUCA | 129 |
| T019_CasPhi.12 | 2 | GUCAGUAUAACCAAAAA | 130 |
| T020_CasPhi.12 | 2 | CUGCAUUUGCAGAGUUU | 131 |
| T021_CasPhi.12 | 2 | UACAUAAUCCAUCUAUU | 132 |
| T022_CasPhi.12 | 2 | AAACUAAACUCUGCAAA | 133 |
| T023_CasPhi.12 | 2 | ACAGAUCUGUUGAGAAA | 134 |
| T024_CasPhi.12 | 2 | GUUUCAAGUAACAAAUA | 135 |
| T025_CasPhi.12 | 2 | UGUAAAACAGAUUAUAG | 136 |
| T026_CasPhi.12 | 2 | UUACUUGAAACUAAACU | 137 |
| T027_CasPhi.12 | 2 | ACAUAAUCCAUCUAUUU | 138 |
| T028_CasPhi.12 | 2 | CAUAAUCCAUCUAUUUU | 139 |
| T029_CasPhi.12 | 2 | UCAGUAUAACCAAAAAA | 140 |
| T030_CasPhi.12 | 2 | UUGAUCCAUAUGCUUUU | 141 |
| T031_CasPhi.12 | 2 | CAUCACCCUUCAGAACC | 142 |
| T032_CasPhi.12 | 2 | AGCACAUCUGGACUCUU | 143 |
| T033_CasPhi.12 | 2 | AAGAGUCCAGAUGUGCU | 144 |
| T034_CasPhi.12 | 2 | AUCUUCAGCACAUCUGG | 145 |
| T035_CasPhi.12 | 2 | GACUGUUGUUGUCAUCA | 146 |
| T036_CasPhi.12 | 2 | AAGAUCAGGUUCUGAAG | 147 |
| T037_CasPhi.12 | 2 | AGAAGUUAAAGAGUCCA | 148 |
| T038_CasPhi.12 | 2 | AAGAAGUUAAAGAGUCC | 149 |
| T039_CasPhi.12 | 2 | CCUUAAGAUACCAUUUG | 150 |
| T040_CasPhi.12 | 2 | UCUUCAGCACAUCUGGA | 151 |
| T041_CasPhi.12 | 2 | UGAAGGGUGAUGGAAAU | 152 |
| T042_CasPhi.12 | 2 | ACUUCUUAAAGAUCAGG | 153 |
| T043_CasPhi.12 | 2 | UGACUGUUGUUGUCAUC | 154 |
| T044_CasPhi.12 | 2 | ACUGUUGUUGUCAUCAU | 155 |
| T045_CasPhi.12 | 2 | UCGAAAUUGUAUUUAUC | 156 |
| T046_CasPhi.12 | 2 | CUAGUAAUAUAAUGAUG | 157 |
| T047_CasPhi.12 | 2 | AGGUAAGUCUUUGAUUU | 158 |
| T048_CasPhi.12 | 2 | UUAAAGAUCAGGUUCUG | 159 |
| T049_CasPhi.12 | 2 | CGAAAUUGUAUUUAUCU | 160 |
| T050_CasPhi.12 | 2 | AACUUCUUAAAGAUCAG | 161 |
| T051_CasPhi.12 | 2 | GUUUUUUCGAAAUUGUA | 162 |
| T052_CasPhi.12 | 2 | UAUUUAUCUUCAGCACA | 163 |
| T053_CasPhi.12 | 2 | UAGUAAUAUAAUGAUGA | 164 |
| T054_CasPhi.12 | 2 | UCAUCAUUAUAUUACUA | 165 |
| T055_CasPhi.12 | 2 | AGAUACCAUUUGUAUUU | 166 |
| T056_CasPhi.12 | 2 | CUAGAAAGAAAAUUAUC | 167 |
| T057_CasPhi.12 | 2 | GAAAUUGUAUUUAUCUU | 168 |
| T058_CasPhi.12 | 2 | UCUUUCUAGUAAUAUAA | 169 |
| T059_CasPhi.12 | 2 | UUCGAAAUUGUAUUUAU | 170 |
| T060_CasPhi.12 | 2 | UUUCGAAAUUGUAUUUA | 171 |
| T061_CasPhi.12 | 2 | UUGUCAUCAUUAUAUUA | 172 |
| T062_CasPhi.12 | 2 | UUUCUAGUAAUAUAAUG | 173 |
| T063_CasPhi.12 | 2 | UAUUACUAGAAAGAAAA | 174 |
| T064_CasPhi.12 | 2 | GAUUUGUUUUUUCGAAA | 175 |
| T065_CasPhi.12 | 2 | UUUUUUCGAAAUUGUAU | 176 |
| T066_CasPhi.12 | 2 | CUUUUGACUGUUGUUGU | 177 |
| T067_CasPhi.12 | 2 | AUUUGUUUUUUCGAAAU | 178 |
| T068_CasPhi.12 | 2 | GCCAGUACAACUGCAUG | 179 |
| T069_CasPhi.12 | 2 | AACAGUGUGCUACCACA | 180 |
| T070_CasPhi.12 | 2 | GGUAUCUUACAGGAACU | 181 |
| T071_CasPhi.12 | 2, | UGGUAUCUUACAGGAAC | 182 |
| | CasPhi.12 | | |
| | L26R Variant | | |
| T072_CasPhi.12 | 2, | GUAUCUUACAGGAACUC | 183 |
| | CasPhi.12 | | |
| | L26R Variant | | |
| T073_CasPhi.12 | 2 | CCAGUACAACUGCAUGU | 184 |
| T074_CasPhi.12 | 2 | UUUGCCAGUACAACUGC | 185 |
| T075_CasPhi.12 | 2 | CAGGAACUCCAGGAUGG | 186 |
| T076_CasPhi.12 | 2 | CUUUGCCAGUACAACUG | 187 |
| T077_CasPhi.12 | 2 | CACAUGGAGCUUUUGUA | 188 |
| T078_CasPhi.12 | 2 | CCCUAUGAAACUGACAU | 189 |
| T079_CasPhi.12 | 2 | CCUAUGAAACUGACAUG | 190 |
| T080_CasPhi.12 | 2 | UUGGUAUCUUACAGGAA | 191 |
| T081_CasPhi.12 | 2 | ACAUGGAGCUUUUGUAU | 192 |
| T082_CasPhi.12 | 2 | CAUAGGGAAAUUUUCAC | 193 |
| T083_CasPhi.12 | 2 | UCACAUGGAGCUUUUGU | 194 |
| T084_CasPhi.12 | 2 | GCCUUUUUGGUAUCUUA | 195 |
| T085_CasPhi.12 | 2 | CCUUUUUGGUAUCUUAC | 196 |
| T086_CasPhi.12 | 2 | CUUUCUUUGCCAGUACA | 197 |
| T087_CasPhi.12 | 2 | AUAGGGAAAUUUUCACA | 198 |
| T088_CasPhi.12 | 2 | UACUGGCAAAGAAAGAA | 199 |
| T089_CasPhi.12 | 2 | UUUCUUUGCCAGUACAA | 200 |
| T090_CasPhi.12 | 2 | GAGAAAAGAUUAAACAG | 201 |
| T091_CasPhi.12 | 2 | UGCCUUUUUGGUAUCUU | 202 |
| T092_CasPhi.12 | 2 | GUUUUGCCUUUUUGGUA | 203 |
| T093_CasPhi.12 | 2 | CUCAAAUAAAAAGACAU | 204 |
| T094_CasPhi.12 | 2 | AGAAAAGAUUAAACAGU | 205 |
| T095_CasPhi.12 | 2 | AAUCUUUUCUCAAAUAA | 206 |
| T096_CasPhi.12 | 2 | UAUUUGAGAAAAGAUUA | 207 |
| T097_CasPhi.12 | 2 | GUAUUUCUUUCUUUGCC | 208 |
| T098_CasPhi.12 | 2 | AUUUGAGAAAAGAUUAA | 209 |
| T099_CasPhi.12 | 2 | UCAAAUAAAAAGACAUG | 210 |
| T100_CasPhi.12 | 2 | AUCUUUUCUCAAAUAAA | 211 |
| T101_CasPhi.12 | 2 | UUAUUUGAGAAAAGAUU | 212 |
| T102_CasPhi.12 | 2 | UUUUGCCUUUUUGGUAU | 213 |
| T103_CasPhi.12 | 2 | UAUUUCUUUCUUUGCCA | 214 |
| T104_CasPhi.12 | 2 | UUGUUUUGCCUUUUUGG | 215 |
| T105_CasPhi.12 | 2 | UCUCAAAUAAAAAGACA | 216 |
| T106_CasPhi.12 | 2 | UGUUUUGCCUUUUUGGU | 217 |
| T107_CasPhi.12 | 2 | UGUAUUUCUUUCUUUGC | 218 |
| T108_CasPhi.12 | 2 | AUUUUUGUUUUGCCUUU | 219 |
| T109_CasPhi.12 | 2 | CUAUUAGAUCUGUCGCC | 220 |
| T110_CasPhi.12 | 2 | GAUCUGUCGCCCUACCU | 221 |
| T111_CasPhi.12 | 2 | ACCCUGCUUAUAAUCUC | 222 |
| T112_CasPhi.12 | 2 | AAGAACCUAUCAACCAU | 223 |
| T113_CasPhi.12 | 2 | GUGCCUUUCACCCUGCU | 224 |
| T114_CasPhi.12 | 2 | UAAGCAGGGUGAAAGGC | 225 |
| T115_CasPhi.12 | 2 | CACCCUGCUUAUAAUCU | 226 |
| T116_CasPhi.12 | 2 | CAUGAGAGAUUAUAAGC | 227 |
| T117_CasPhi.12 | 2 | UAGCAGACUUUUUAAGC | 228 |
| T118_CasPhi.12 | 2 | AUUAAUGGUUGAUAGGU | 229 |
| T119_CasPhi.12 | 2 | AUGUUAGUGCCUUUCAC | 230 |
| T120_CasPhi.12 | 2 | ACUGCUGUUGAUUAAUG | 231 |
| T121_CasPhi.12 | 2 | CUGCUGUUGAUUAAUGG | 232 |
| T122_CasPhi.12 | 2 | UGUCUGACAGCUGUUUG | 233 |
| T123_CasPhi.12 | 2 | AGCAGACUUUUUAAGCU | 234 |
| T124_CasPhi.12 | 2 | AUCAACAGCAGUAAAGA | 235 |
| T125_CasPhi.12 | 2 | CAUUCCUAUUAGAUCUG | 236 |
| T126_CasPhi.12 | 2 | UUCUGUCUGACAGCUGU | 237 |
| T127_CasPhi.12 | 2 | AUGAGAGAUUAUAAGCA | 238 |
| T128_CasPhi.12 | 2 | UCUGUCUGACAGCUGUU | 239 |
| T129_CasPhi.12 | 2 | GCAGACUUUUUAAGCUU | 240 |
| T130_CasPhi.12 | 2 | AUGGUUGAUAGGUUCUU | 241 |
| T131_CasPhi.12 | 2 | CUGUCUGACAGCUGUUU | 242 |
| T132_CasPhi.12 | 2 | AAUGUUAGUGCCUUUCA | 243 |
| T133_CasPhi.12 | 2 | AUUCCUAUUAGAUCUGU | 244 |
| T134_CasPhi.12 | 2 | UUUCUGUCUGACAGCUG | 245 |
| T135_CasPhi.12 | 2 | GAAGAAUAUUUCAUGAG | 246 |
| T136_CasPhi.12 | 2 | UUUAAUGUUAGUGCCUU | 247 |
| T137_CasPhi.12 | 2 | AGAAGAAUAUUUCAUGA | 248 |
| T138_CasPhi.12 | 2 | UCAUUCCUAUUAGAUCU | 249 |
| T139_CasPhi.12 | 2 | UUUACUGCUGUUGAUUA | 250 |
| T140_CasPhi.12 | 2 | UAAGCUUUCUUUAGAAG | 251 |
| T141_CasPhi.12 | 2 | AGCUUUCUUUAGAAGAA | 252 |
| T142_CasPhi.12 | 2 | CUUUACUGCUGUUGAUU | 253 |
| T143_CasPhi.12 | 2 | UUCUAAAGAAAGCUUAA | 254 |
| T144_CasPhi.12 | 2 | AUAGGUUCUUUAAUGUU | 255 |
| T145_CasPhi.12 | 2 | AAAAGUCUGCUAAAAUG | 256 |
| T146_CasPhi.12 | 2 | AAGCUUUCUUUAGAAGA | 257 |
| T147_CasPhi.12 | 2 | UUAAGCUUUCUUUAGAA | 258 |
| T148_CasPhi.12 | 2 | UAAUCUCUCAUGAAAUA | 259 |
| T149_CasPhi.12 | 2 | CUUUAGAAGAAUAUUUC | 260 |
| T150_CasPhi.12 | 2 | UUUAGAAGAAUAUUUCA | 261 |
| T151_CasPhi.12 | 2 | AUCGAAUAAGUAAUGUG | 262 |
| T152_CasPhi.12 | 2 | CUUAUUCGAUUAACACU | 263 |
| T153_CasPhi.12 | 2 | UUCGAUUAACACUUUGA | 264 |
| T154_CasPhi.12 | 2 | GAAGAUCUGAGCUCUGA | 265 |
| T155_CasPhi.12 | 2 | UACCCACUAAAGUUAAU | 266 |
| T156_CasPhi.12 | 2 | ACCAAAUGGAUUAAGAU | 267 |
| T157_CasPhi.12 | 2 | AAAGUGUUAAUCGAAUA | 268 |
| T158_CasPhi.12 | 2 | AAAGAAAUUCUACCCAC | 269 |
| T159_CasPhi.12 | 2 | AGUGGGUAGAAUUUCUU | 270 |
| T160_CasPhi.12 | 2 | GAUUAACACUUUGAAGA | 271 |
| T161_CasPhi.12 | 2 | AUGAACAUCUUAAUCCA | 272 |
| T162_CasPhi.12 | 2 | GGUGAAUAUAUUAUUGG | 273 |
| T163_CasPhi.12 | 2 | AACAGAAAAGCAUACAC | 274 |
| T164_CasPhi.12 | 2 | UCUGUUAAAGAGGAAGU | 275 |
| T165_CasPhi.12 | 2 | UGUUAAAGAGGAAGUUA | 276 |
| T166_CasPhi.12 | 2 | UCUUCAAAGUGUUAAUC | 277 |
| T167_CasPhi.12 | 2 | CUGUUAAAGAGGAAGUU | 278 |
| T168_CasPhi.12 | 2 | GUGAAUAUAUUAUUGGA | 279 |
| T169_CasPhi.12 | 2 | AUCCAUUUGGUGAAUAU | 280 |
| T170_CasPhi.12 | 2 | GUGGGUAGAAUUUCUUU | 281 |
| T171_CasPhi.12 | 2 | AAGAGGAAGUUAGAAGA | 282 |
| T172_CasPhi.12 | 2 | UAACUUCCUCUUUAACA | 283 |
| T173_CasPhi.12 | 2 | AAGAUAAUUCAUGAACA | 284 |
| T174_CasPhi.12 | 2 | AGAUGUUCAUGAAUUAU | 285 |
| T175_CasPhi.12 | 2 | UAAAUUAACUUUAGUGG | 286 |
| T176_CasPhi.12 | 2 | AAAAGAAAUUCUACCCA | 287 |
| T177_CasPhi.12 | 2 | CUCUUUAACAGAAAAGC | 288 |
| T178_CasPhi.12 | 2 | GAAGUAAAAUAUAAUAG | 289 |
| T179_CasPhi.12 | 2 | ACAGAAAAGCAUACACA | 290 |
| T180_CasPhi.12 | 2 | AUGAAUUAUCUUCAAAG | 291 |
| T181_CasPhi.12 | 2 | ACACUUUGAAGAUAAUU | 292 |
| T182_CasPhi.12 | 2 | UACUUCUAAAUUAACUU | 293 |
| T183_CasPhi.12 | 2 | CUUCUAAAUUAACUUUA | 294 |
| T184_CasPhi.12 | 2 | ACUUCUAAAUUAACUUU | 295 |
| T185_CasPhi.12 | 2 | AGAAGUAAAAUAUAAUA | 296 |
| T186_CasPhi.12 | 2 | AUUUAGAAGUAAAAUAU | 297 |
| T187_CasPhi.12 | 2 | CUUUAAAAGAAAUUCUA | 298 |
| T188_CasPhi.12 | 2 | UUGGAUUUCUAUUAUAU | 299 |
| T189_CasPhi.12 | 2 | CUAUUAUAUUUUACUUC | 300 |
| T190_CasPhi.12 | 2 | GAUUUCUAUUAUAUUUU | 301 |
| T191_CasPhi.12 | 2 | UAUUUUACUUCUAAAUU | 302 |
| T192_CasPhi.12 | 2 | UAUUAUAUUUUACUUCU | 303 |
| T193_CasPhi.12 | 2 | CAGGCUCCAAUAGUGGU | 304 |
| T194_CasPhi.12 | 2 | GAGCCUGUAAGUAUACU | 305 |
| T195_CasPhi.12 | 2 | AACAAAUAGCUAGAGCC | 306 |
| T196_CasPhi.12 | 2 | GUGUUAUUGACUGUGCA | 307 |
| T197_CasPhi.12 | 2 | GAACAAAUAGCUAGAGC | 308 |
| T198_CasPhi.12 | 2 | GGCUCUAGCUAUUUGUU | 309 |
| T199_CasPhi.12 | 2 | GCACAGUCAAUAACACA | 310 |
| T200_CasPhi.12 | 2 | UGCACAGUCAAUAACAC | 311 |
| T201_CasPhi.12 | 2 | GUUCAAAAGUGCAACUA | 312 |
| T202_CasPhi.12 | 2 | GCUCUAGCUAUUUGUUC | 313 |
| T203_CasPhi.12 | 2 | UUGCACAGUCAAUAACA | 314 |
| T204_CasPhi.12 | 2 | UGAACAAAUAGCUAGAG | 315 |
| T205_CasPhi.12 | 2 | UGUUAUUGACUGUGCAA | 316 |
| T206_CasPhi.12 | 2 | CACAGUCAAUAACACAA | 317 |
| T207_CasPhi.12 | 2 | UCUCUCACCCAGUCAUC | 318 |
| T208_CasPhi.12 | 2 | UUGACUGUGCAAAAAGU | 319 |
| T209_CasPhi.12 | 2 | UCUUUGGCUCUAGCUAU | 320 |
| T210_CasPhi.12 | 2 | UCUCUCUCACCCAGUCA | 321 |
| T211_CasPhi.12 | 2 | ACUGUGCAAAAAGUCUU | 322 |
| T212_CasPhi.12 | 2 | UUCAAAAGUGCAACUAU | 323 |
| T213_CasPhi.12 | 2 | AUAGUUGCACUUUUGAA | 324 |
| T214_CasPhi.12 | 2 | CAAUUCUAAAGAUAGAG | 325 |
| T215_CasPhi.12 | 2 | GUUUCAAUUCUAAAGAU | 326 |
| T216_CasPhi.12 | 2 | CACUUUUGAACAAAUAG | 327 |
| T217_CasPhi.12 | 2 | AAAAGUGCAACUAUGAA | 328 |
| T218_CasPhi.12 | 2 | CUCUCUCACCCAGUCAU | 329 |
| T219_CasPhi.12 | 2 | AGAAUUGAAACAAAUUU | 330 |
| T220_CasPhi.12 | 2 | GAAUUGAAACAAAUUUU | 331 |
| T221_CasPhi.12 | 2 | UCUCUAUCUUUAGAAUU | 332 |
| T222_CasPhi.12 | 2 | AAUUCUAAAGAUAGAGA | 333 |
| T223_CasPhi.12 | 2 | UAAAGAUAGAGAUAAAC | 334 |
| T224_CasPhi.12 | 2 | AAACAAAUUUUCUCUCU | 335 |
| T225_CasPhi.12 | 2 | UUUCAAUUCUAAAGAUA | 336 |
| T226_CasPhi.12 | 2, 5 | GCUCCUACUCAGACUGU | 337 |
| T227_CasPhi.12 | 2 | AGCUCCUACUCAGACUG | 338 |
| T228_CasPhi.12 | 2 | GGCUCAAAUUGUUACUC | 339 |
| T229_CasPhi.12 | 2 | UAGCUCCUACUCAGACU | 340 |
| T230_CasPhi.12 | 2 | GAGCCAAACUCUUAUUC | 341 |
| T231_CasPhi.12 | 2 | GGGUUUUUGCAAAAAGG | 342 |
| T232_CasPhi.12 | 2 | GCUCAAAUUGUUACUCU | 343 |
| T233_CasPhi.12 | 2 | AAAUUGGCUCUUUAGCU | 344 |
| T234_CasPhi.12 | 2 | GCUCUUUAGCUUGUGUU | 345 |
| T235_CasPhi.12 | 2 | AGCCAAACUCUUAUUCA | 346 |
| T236_CasPhi.12 | 2 | CCUUUUUGCAAAAACCC | 347 |
| T237_CasPhi.12 | 2 | AAGAGUAACAAUUUGAG | 348 |
| T238_CasPhi.12 | 2 | GAAACACAAGCUAAAGA | 349 |
| T239_CasPhi.12 | 2 | GACUUAUUGUUAUUGAA | 350 |
| T240_CasPhi.12 | 2 | GCAAAAACCCAAAAUAU | 351 |
| T241_CasPhi.12 | 2 | GGUUUUUGCAAAAAGGA | 352 |
| T242_CasPhi.12 | 2 | UCCUUUUUGCAAAAACC | 353 |
| T243_CasPhi.12 | 2 | CAAUAACAAUAAGUCAA | 354 |
| T244_CasPhi.12 | 2 | AUUGAAGAGUAACAAUU | 355 |
| T245_CasPhi.12 | 2 | UUGAAAUUGGCUCUUUA | 356 |
| T246_CasPhi.12 | 2 | UGCAAAAACCCAAAAUA | 357 |
| T247_CasPhi.12 | 2 | UUAUUGAAAUUGGCUCU | 358 |
| T248_CasPhi.12 | 2 | AGCUUGUGUUUCUAAUU | 359 |
| T249_CasPhi.12 | 2 | UUGCAAAAACCCAAAAU | 360 |
| T250_CasPhi.12 | 2 | UUGUUAUUGAAAUUGGC | 361 |
| T251_CasPhi.12 | 2 | GCUUGUGUUUCUAAUUU | 362 |
| T252_CasPhi.12 | 2 | UUCAUGACAUUAUAUAU | 363 |
| T253_CasPhi.12 | 2 | CUCUUCAAUUAAAUUUG | 364 |
| T254_CasPhi.12 | 2 | UUACUCUUCAAUUAAAU | 365 |
| T255_CasPhi.12 | 2 | CAAAAACCCAAAAUAUU | 366 |
| T256_CasPhi.12 | 2 | UUCCUUUUUGCAAAAAC | 367 |
| T257_CasPhi.12 | 2 | CUUUUUGCAAAAACCCA | 368 |
| T258_CasPhi.12 | 2 | ACUUAUUGUUAUUGAAA | 369 |
| T259_CasPhi.12 | 2 | AAUUUGACUUAUUGUUA | 370 |
| T260_CasPhi.12 | 2 | AAUUAAAUUUGACUUAU | 371 |
| T261_CasPhi.12 | 2 | UUUCCUUUUUGCAAAAA | 372 |
| T262_CasPhi.12 | 2 | GCAAAAAGGAAAAAAGA | 373 |
| T263_CasPhi.12 | 2 | UCUUUUUCUUCUUUUUU | 374 |
| T264_CasPhi.12 | 2 | CAAAAAGGAAAAAAGAA | 375 |
| T265_CasPhi.12 | 2 | UUUUUUCCUUUUUGCAA | 376 |
| T266_CasPhi.12 | 2 | UUCUUUUUUCCUUUUUG | 377 |
| T267_CasPhi.12 | 2 | UGUUUCUAAUUUUUCUU | 378 |
| T268_CasPhi.12 | 2 | CUAAUUUUUCUUUUUCU | 379 |
| T269_CasPhi.12 | 2 | UUUUUCUUCUUUUUUCC | 380 |
| T270_CasPhi.12 | 2 | UCUUCUUUUUUCCUUUU | 381 |
| T271_CasPhi.12 | 2 | UAAUUUUUCUUUUUCUU | 382 |
| T272_CasPhi.12 | 2 | UUCUUCUUUUUUCCUUU | 383 |
| T273_CasPhi.12 | 2 | CUUCUUUUUUCCUUUUU | 384 |
| T274_CasPhi.12 | 2 | CUUUUUCUUCUUUUUUC | 385 |
| T275_CasPhi.12 | 2 | UUCUUUUUCUUCUUUUU | 386 |
| T276_CasPhi.12 | 2 | CCCACCAGUUCUUAGGC | 387 |
| T277_CasPhi.12 | 2 | CCUAAGAACUGGUGGGA | 388 |
| T278_CasPhi.12 | 2 | CCACCAGUUCUUAGGCA | 389 |
| T279_CasPhi.12 | 2 | AUGAUCAUCUCGUUGAU | 390 |
| T280_CasPhi.12 | 2 | UGCUUGAUGAUCAUCUC | 391 |
| T281_CasPhi.12 | 2 | CACCAAUCACUUUACUC | 392 |
| T282_CasPhi.12 | 2 | UCCACCAAUCACUUUAC | 393 |
| T283_CasPhi.12 | 2 | ACUCUCCUAGACCAUUU | 394 |
| T284_CasPhi.12 | 2 | CCACCAAUCACUUUACU | 395 |
| T285_CasPhi.12 | 2 | CUCUCCUAGACCAUUUC | 396 |
| T286_CasPhi.12 | 2 | GCUGAGAGAGAAACAGU | 397 |
| T287_CasPhi.12 | 2 | CUCUCACUAUUCAGCCU | 398 |
| T288_CasPhi.12 | 2 | UCAUACCUUCUGCUUGA | 399 |
| T289_CasPhi.12 | 2 | UUAGGCAACUGUUUCUC | 400 |
| T290_CasPhi.12 | 2 | CUCAUACCUUCUGCUUG | 401 |
| T291_CasPhi.12 | 2 | GGCAACUGUUUCUCUCU | 402 |
| T292_CasPhi.12 | 2 | CUCUCUCAGCAAACACA | 403 |
| T293_CasPhi.12 | 2 | AAAGAAAAACUUCUGCC | 404 |
| T294_CasPhi.12 | 2 | UCUCUCAGCAAACACAU | 405 |
| T295_CasPhi.12 | 2 | AAGAAAAACUUCUGCCA | 406 |
| T296_CasPhi.12 | 2 | UUCUCAUACCUUCUGCU | 407 |
| T297_CasPhi.12 | 2 | CUGAGAGAGAAACAGUU | 408 |
| T298_CasPhi.12 | 2 | GCAGAAGUUUUUCUUUA | 409 |
| T299_CasPhi.12 | 2 | UAAAGAAAAACUUCUGC | 410 |
| T300_CasPhi.12 | 2 | GUGGAAAAUCUUCAUUU | 411 |
| T301_CasPhi.12 | 2 | UUUCUCAUACCUUCUGC | 412 |
| T302_CasPhi.12 | 2 | AAAUGAAGAUUUUCCAC | 413 |
| T303_CasPhi.12 | 2 | UGCCAACUUUUAUCAUU | 414 |
| T304_CasPhi.12 | 2 | UCUCAUACCUUCUGCUU | 415 |
| T305_CasPhi.12 | 2 | AAAAUGAAGAUUUUCCA | 416 |
| T306_CasPhi.12 | 2 | UCAUUUUUUCUCAUACC | 417 |
| T307_CasPhi.12 | 2 | UAUCAUUUUUUCUCAUA | 418 |
| T308_CasPhi.12 | 2 | AUCAUUUUUUCUCAUAC | 419 |
| T309_CasPhi.12 | 2 | CUUUAAAAUGAAGAUUU | 420 |
| T310_CasPhi.12 | 2 | UCUUUAAAAUGAAGAUU | 421 |
| T311_CasPhi.12 | 2 | UUCUUUAAAAUGAAGAU | 422 |
| T312_CasPhi.12 | 2 | AUUUUAAAGAAAAACUU | 423 |
| T313_CasPhi.12 | 2 | UUUAAAAUGAAGAUUUU | 424 |
| T314_CasPhi.12 | 2 | AUCAUCCUAGCCAUAAC | 425 |
| T315_CasPhi.12 | 2 | UGUUAUGGCUAGGAUGA | 426 |
| T316_CasPhi.12 | 2 | AAUGUAACUUCCAAACG | 427 |
| T317_CasPhi.12 | 2 | AUUGUGUUAUGGCUAGG | 428 |
| T318_CasPhi.12 | 2 | UUCAUCAUCCUAGCCAU | 429 |
| T319_CasPhi.12 | 2 | AUCCCACUGAUUCUGAA | 430 |
| T320_CasPhi.12 | 2 | AAAUGUCUCCUCCAGAC | 431 |
| T321_CasPhi.12 | 2 | UUCAUUGUGUUAUGGCU | 432 |
| T322_CasPhi.12 | 2 | AGAAUCAGUGGGAUGAA | 433 |
| T323_CasPhi.12 | 2 | AAAAUGUCUCCUCCAGA | 434 |
| T324_CasPhi.12 | 2 | CAAACGUUAUCUCACAU | 435 |
| T325_CasPhi.12 | 2 | GGAAGUUACAUUUAAAA | 436 |
| T326_CasPhi.12 | 2 | UGAAUUCUUUCAACUAG | 437 |
| T327_CasPhi.12 | 2 | CAACUAGAAUAAAAGGA | 438 |
| T328_CasPhi.12 | 2 | UCUCACAUUUAUGUUGC | 439 |
| T329_CasPhi.12 | 2 | AUUCUAGUUGAAAGAAU | 440 |
| T330_CasPhi.12 | 2 | CAUUUAAAAUGUCUCCU | 441 |
| T331_CasPhi.12 | 2 | UAGUUGAAAGAAUUCAG | 442 |
| T332_CasPhi.12 | 2 | AAAGAAUUCAGAAUCAG | 443 |
| T333_CasPhi.12 | 2 | AAUAAGCAACAUAAAUG | 444 |
| T334_CasPhi.12 | 2 | AACUAGAAUAAAAGGAA | 445 |
| T335_CasPhi.12 | 2 | CCUUUUAUUCUAGUUGA | 446 |
| T336_CasPhi.12 | 2 | UUCUAGUUGAAAGAAUU | 447 |
| T337_CasPhi.12 | 2 | UAUUCUAGUUGAAAGAA | 448 |
| T338_CasPhi.12 | 2 | UUAAAUAAGCAACAUAA | 449 |
| T339_CasPhi.12 | 2 | UAAAUAAGCAACAUAAA | 450 |
| T340_CasPhi.12 | 2 | UUUAAAUAAGCAACAUA | 451 |
| T341_CasPhi.12 | 2 | AAAAAUUAUUCAUUGUG | 452 |
| T342_CasPhi.12 | 2 | AAAUAAGCAACAUAAAU | 453 |
| T343_CasPhi.12 | 2 | UCCUUUUAUUCUAGUUG | 454 |
| T344_CasPhi.12 | 2 | GAAGUUACAUUUAAAAU | 455 |
| T345_CasPhi.12 | 2 | CUUUUAUUCUAGUUGAA | 456 |
| T346_CasPhi.12 | 2 | ACUACUAUAUAUUUAUU | 457 |
| T347_CasPhi.12 | 2 | AAAAAAUUAUUCAUUGU | 458 |
| T348_CasPhi.12 | 2 | UUCCUUUUAUUCUAGUU | 459 |
| T349_CasPhi.12 | 2 | CUACUAUAUAUUUAUUU | 460 |
| T350_CasPhi.12 | 2 | UUUUUCCUUUUAUUCUA | 461 |
| T351_CasPhi.12 | 2 | AUUUUUCCUUUUAUUCU | 462 |
| T352_CasPhi.12 | 2 | CAGCUUUAACGUGAUUU | 463 |
| T353_CasPhi.12 | 2 | GAUACUAACCUUGGUUU | 464 |
| T354_CasPhi.12 | 2 | AUUUCAGCUUUAACGUG | 465 |
| T355_CasPhi.12 | 2 | AUACUAACCUUGGUUUC | 466 |
| T356_CasPhi.12 | 2 | GUAUCAAAGAUACCUUU | 467 |
| T357_CasPhi.12 | 2 | ACAGAAAAUCACGUUAA | 468 |
| T358_CasPhi.12 | 2 | UUAACAGAAAAUCACGU | 469 |
| T359_CasPhi.12 | 2 | UAUCAAAUGUAACCAGU | 470 |
| T360_CasPhi.12 | 2 | AAUUACAAUCUAUGGUA | 471 |
| T361_CasPhi.12 | 2 | AUCAAAUGUAACCAGUA | 472 |
| T362_CasPhi.12 | 2 | AGCUUUAACGUGAUUUU | 473 |
| T363_CasPhi.12 | 2 | ACGUGAUUUUCUGUUAA | 474 |
| T364_CasPhi.12 | 2 | AACGUGAUUUUCUGUUA | 475 |
| T365_CasPhi.12 | 2 | UCAAAUGUAACCAGUAU | 476 |
| T366_CasPhi.12 | 2 | UACCAUAGAUUGUAAUU | 477 |
| T367_CasPhi.12 | 2 | CAAUCUAUGGUAUAAUU | 478 |
| T368_CasPhi.12 | 2 | AAGCUGAAAUGAACAGU | 479 |
| T369_CasPhi.12 | 2 | GAUAAAAUUAUACCAUA | 480 |
| T370_CasPhi.12 | 2 | CUGUUAAUAACUUUACA | 481 |
| T371_CasPhi.12 | 2 | UCUGUUAAUAACUUUAC | 482 |
| T372_CasPhi.12 | 2 | AAAUAAAAUACUGGUUA | 483 |
| T373_CasPhi.12 | 2 | AAUGAUGUUUAAUGUAA | 484 |
| T374_CasPhi.12 | 2 | AAAAGGUAUCUUUGAUA | 485 |
| T375_CasPhi.12 | 2 | ACAUUAAACAUCAUUAA | 486 |
| T376_CasPhi.12 | 2 | AUGUAAAGUUAUUAACA | 487 |
| T377_CasPhi.12 | 2 | AUAAAAUUAUACCAUAG | 488 |
| T378_CasPhi.12 | 2 | UAAAAAGGUAUCUUUGA | 489 |
| T379_CasPhi.12 | 2 | AAAAAGGUAUCUUUGAU | 490 |
| T380_CasPhi.12 | 2 | AAUGUAAAGUUAUUAAC | 491 |
| T381_CasPhi.12 | 2 | AACAUCAUUAAAUUACA | 492 |
| T382_CasPhi.12 | 2 | AUGAUGUUUAAUGUAAA | 493 |
| T383_CasPhi.12 | 2 | AAAAUAAAAUACUGGUU | 494 |
| T384_CasPhi.12 | 2 | UUAAAAUAAAAUACUGG | 495 |
| T385_CasPhi.12 | 2 | UAAAAUAAAAUACUGGU | 496 |
| T386_CasPhi.12 | 2 | CUGUGAUUUUCUUUUGG | 497 |
| T387_CasPhi.12 | 2 | UGUUAAUAACUUUACAU | 498 |
| T388_CasPhi.12 | 2 | UAUUUUAAAAAGGUAUC | 499 |
| T389_CasPhi.12 | 2 | UAAUUUAAUGAUGUUUA | 500 |
| T390_CasPhi.12 | 2 | GUUUCUGUGAUUUUCUU | 501 |
| T391_CasPhi.12 | 2 | AUUUUAAAAAGGUAUCU | 502 |
| T392_CasPhi.12 | 2 | UUUUAAAAAGGUAUCUU | 503 |
| T393_CasPhi.12 | 2 | CAUUUGAUAAAAUUAUA | 504 |
| T394_CasPhi.12 | 2 | UGUGAUUUUCUUUUGGA | 505 |
| T395_CasPhi.12 | 2 | CAUCUACUGUAUAGGGA | 506 |
| T396_CasPhi.12 | 2 | GCUCUGGCCUGUCCUAA | 507 |
| T397_CasPhi.12 | 2 | CAUUCAACUGUUGCCUC | 508 |
| T398_CasPhi.12 | 2 | UGAAGGUGUUCUUGUAC | 509 |
| T399_CasPhi.12 | 2 | CUUUUGGAUUGCAUCUA | 510 |
| T400_CasPhi.12 | 2 | CUUAGCUUCCAGCCAUU | 511 |
| T401_CasPhi.12 | 2 | UUUUGGAUUGCAUCUAC | 512 |
| T402_CasPhi.12 | 2 | GGAUUGCAUCUACUGUA | 513 |
| T403_CasPhi.12 | 2 | AACAUUUCAUUCAACUG | 514 |
| T404_CasPhi.12 | 2 | UUGUACUUCAUCCCACU | 515 |
| T405_CasPhi.12 | 2 | UCUUUUGGAUUGCAUCU | 516 |
| T406_CasPhi.12 | 2 | ACAUUUCAUUCAACUGU | 517 |
| T407_CasPhi.12 | 2 | UUCCUUAGCUUCCAGCC | 518 |
| T408_CasPhi.12 | 2 | GAUUGCAUCUACUGUAU | 519 |
| T409_CasPhi.12 | 2 | CAUGACUCAAGCUUGGC | 520 |
| T410_CasPhi.12 | 2 | AUUCAACUGUUGCCUCC | 521 |
| T411_CasPhi.12 | 2 | UGUUGAAUCCUUUAACA | 522 |
| T412_CasPhi.12 | 2 | UGGAUUGCAUCUACUGU | 523 |
| T413_CasPhi.12 | 2 | GCUUCCAGCCAUUGUGU | 524 |
| T414_CasPhi.12 | 2 | CAGCCAUUGUGUUGAAU | 525 |
| T415_CasPhi.12 | 2 | CCUCCGGUUCUGAAGGU | 526 |
| T416_CasPhi.12 | 2 | UACUUCAUCCCACUGAU | 527 |
| T417_CasPhi.12 | 2 | AACUGUUGCCUCCGGUU | 528 |
| T418_CasPhi.12 | 2 | AAGGAUUCAACACAAUG | 529 |
| T419_CasPhi.12 | 2 | AACACAAUGGCUGGAAG | 530 |
| T420_CasPhi.12 | 2 | GGACAGGCCAGAGCCAA | 531 |
| T421_CasPhi.12 | 2 | AGUCAUGGAAGGAGGGU | 532 |
| T422_CasPhi.12 | 2 | AGAACCGGAGGCAACAG | 533 |
| T423_CasPhi.12 | 2 | UAUAACUUGAUCAAGCA | 534 |
| T424_CasPhi.12 | 2 | AUCAAGCAGAGAAAGCC | 535 |
| T425_CasPhi.12 | 2 | UAGUUUGGAGAUGGCAG | 536 |
| T426_CasPhi.12 | 2, 5 | GAGAUGGCAGUUUCCUU | 537 |
| T427_CasPhi.12 | 2 | CCUUAGUAACCACAGGU | 538 |
| T428_CasPhi.12 | 2 | AUAUCCUCAAGGUCACC | 539 |
| T429_CasPhi.12 | 2 | CUUAGUAACCACAGGUU | 540 |
| T430_CasPhi.12 | 2 | UGUCCAAGCCCGGUUGA | 541 |
| T431_CasPhi.12 | 2 | CUAGUUUGGAGAUGGCA | 542 |
| T432_CasPhi.12 | 2 | AAAUCUGCCAGAGCAGG | 543 |
| T433_CasPhi.12 | 2 | UAACUUGAUCAAGCAGA | 544 |
| T434_CasPhi.12 | 2 | GUAACCACAGGUUGUGU | 545 |
| T435_CasPhi.12 | 2 | UGUCACCAGAGUAACAG | 546 |
| T436_CasPhi.12 | 2 | AUAACUUGAUCAAGCAG | 547 |
| T437_CasPhi.12 | 2 | GGAGAUGGCAGUUUCCU | 548 |
| T438_CasPhi.12 | 2 | CUCUGGUGACACAACCU | 549 |
| T439_CasPhi.12 | 2 | AUGUUGGAGGUACCUGC | 550 |
| T440_CasPhi.12 | 2 | CUCUGCUUGAUCAAGUU | 551 |
| T441_CasPhi.12 | 2 | UCUGCUUGAUCAAGUUA | 552 |
| T442_CasPhi.12 | 2 | CUUGAUGUUGGAGGUAC | 553 |
| T443_CasPhi.12 | 2 | AACCGGGCUUGGACAGA | 554 |
| T444_CasPhi.12 | 2 | AGGAUAUCAACGAGAUG | 555 |
| T445_CasPhi.12 | 2 | GAGGUACCUGCUCUGGC | 556 |
| T446_CasPhi.12 | 2 | CAACCGGGCUUGGACAG | 557 |
| T447_CasPhi.12 | 2 | CUAAGGAAACUGCCAUC | 558 |
| T448_CasPhi.12 | 2 | UAAAAUCACAGAGGGUG | 559 |
| T449_CasPhi.12 | 2 | GACAGAACUUACCGACU | 560 |
| T450_CasPhi.12 | 2 | CCGACUGGCUUUCUCUG | 561 |
| T451_CasPhi.12 | 2 | CCGCCUUCCACUCAGAG | 562 |
| T452_CasPhi.12 | 2 | CCCUCAGCUCUUGAAGU | 563 |
| T453_CasPhi.12 | 2 | AAGUAAACGGUUUACCG | 564 |
| T454_CasPhi.12 | 2 | ACCGCCUUCCACUCAGA | 565 |
| T455_CasPhi.12 | 2 | GCCCUCAGCUCUUGAAG | 566 |
| T456_CasPhi.12 | 2 | CACUCAGAGCUCAGAUC | 567 |
| T457_CasPhi.12 | 2 | CUUCAAGAGCUGAGGGC | 568 |
| T458_CasPhi.12 | 2 | ACUUCAAGAGCUGAGGG | 569 |
| T459_CasPhi.12 | 2 | AAGAGCUGAGGGCAAAG | 570 |
| T460_CasPhi.12 | 2 | UCCUGUAGAAUACUGGC | 571 |
| T461_CasPhi.12 | 2 | GAGGAUUGCUGAAUUAU | 572 |
| T462_CasPhi.12 | 2 | GCCGCUGCCCAAUGCCA | 573 |
| T463_CasPhi.12 | 2 | UUCCCCAGUUGCAUUCA | 574 |
| T464_CasPhi.12 | 2 | UGAGGAUUGCUGAAUUA | 575 |
| T465_CasPhi.12 | 2 | CCGCUGCCCAAUGCCAU | 576 |
| T466_CasPhi.12 | 2 | AGGAUUGCUGAAUUAUU | 577 |
| T467_CasPhi.12 | 2 | CCAAUUUUUCCUGUAGA | 578 |
| T468_CasPhi.12 | 2 | UUCCUGUAGAAUACUGG | 579 |
| T469_CasPhi.12 | 2 | GCAGACCUCCUGCCACC | 580 |
| T470_CasPhi.12 | 2 | CAUUCAAUGUUCUGACA | 581 |
| T471_CasPhi.12 | 2 | UGACAACAGUUUGCCGC | 582 |
| T472_CasPhi.12 | 2 | CUUCCCCAGUUGCAUUC | 583 |
| T473_CasPhi.12 | 2 | CCUGUAGAAUACUGGCA | 584 |
| T474_CasPhi.12 | 2 | CUGAAUUAUUUCUUCCC | 585 |
| T475_CasPhi.12 | 2 | CUGUAGAAUACUGGCAU | 586 |
| T476_CasPhi.12 | 2 | UUGAGGAUUGCUGAAUU | 587 |
| T477_CasPhi.12 | 2 | AAUGUUCUGACAACAGU | 588 |
| T478_CasPhi.12 | 2 | CCCAGUUGCAUUCAAUG | 589 |
| T479_CasPhi.12 | 2 | CAGACCUCCUGCCACCG | 590 |
| T480_CasPhi.12 | 2 | UUUCUUCCCCAGUUGCA | 591 |
| T481_CasPhi.12 | 2 | AGGCUUCCCAAUUUUUC | 592 |
| T482_CasPhi.12 | 2 | GGAAGCCUGAAUCUGCG | 593 |
| T483_CasPhi.12 | 2 | AGCAAUCCUCAAAAACA | 594 |
| T484_CasPhi.12 | 2 | UCAGAACAUUGAAUGCA | 595 |
| T485_CasPhi.12 | 2 | AAUGCAACUGGGGAAGA | 596 |
| T486_CasPhi.12 | 2 | GGCAGCGGCAAACUGUU | 597 |
| T487_CasPhi.12 | 2 | UACAGGAAAAAUUGGGA | 598 |
| T488_CasPhi.12 | 2 | GCCACUGAUUAAAUAUC | 599 |
| T489_CasPhi.12 | 2 | GUGUCUUUCUGAGAAAC | 600 |
| T490_CasPhi.12 | 2 | UCAACAGAUCUGUCAAA | 601 |
| T491_CasPhi.12 | 2 | GUAUUUAGCAUGUUCCC | 602 |
| T492_CasPhi.12 | 2 | UAUUUAGCAUGUUCCCA | 603 |
| T493_CasPhi.12 | 2 | UGUUAGCCACUGAUUAA | 604 |
| T494_CasPhi.12 | 2 | UGAGAAACUGUUCAGCU | 605 |
| T495_CasPhi.12 | 2 | AGCAUGUUCCCAAUUCU | 606 |
| T496_CasPhi.12 | 2 | UAUCAUAAUGAAAACGC | 607 |
| T497_CasPhi.12 | 2 | CUGAGAAACUGUUCAGC | 608 |
| T498_CasPhi.12 | 2 | CCAAUUCUCAGGAAUUU | 609 |
| T499_CasPhi.12 | 2 | UGUCUUUCUGAGAAACU | 610 |
| T500_CasPhi.12 | 2 | UCAGGAAUUUGUGUCUU | 611 |
| T501_CasPhi.12 | 2 | GCAUGUUCCCAAUUCUC | 612 |
| T502_CasPhi.12 | 2 | AGCUUCUGUUAGCCACU | 613 |
| T503_CasPhi.12 | 2 | UCAGAAAGACACAAAUU | 614 |
| T504_CasPhi.12 | 2 | AAUCAGUGGCUAACAGA | 615 |
| T505_CasPhi.12 | 2 | AGAAAUGGCGGCGUUUU | 616 |
| T506_CasPhi.12 | 2 | AUCAGUGGCUAACAGAA | 617 |
| T507_CasPhi.12 | 2 | GGAACAUGCUAAAUACA | 618 |
| T508_CasPhi.12 | 2 | CUGAGAAUUGGGAACAU | 619 |
| T509_CasPhi.12 | 2 | CUCAGAAAGACACAAAU | 620 |
| | CasPhi.12 | UGGUAUCUUAmCmAmG | 621 |
| | (SEQ ID NO: 2) | | |
| | L26R Variant | | |
| T01_CasM.19952 | 3 | AAAUUGUAUUUAUCUUCAGC | 622 |
| T02_CasM.19952 | 3 | AAAAAACAAAUCAAAGACUU | 623 |
| T03_CasM.19952 | 3 | CCUGCAGGUAAAAGCAUAUG | 624 |
| T04_CasM.19952 | 3 | CCCUACCUCUUUUUUCUGUC | 625 |
| T05_CasM.19952 | 3 | AAUAAGUAAUGUGUAUGCUU | 626 |
| T06_CasM.19952 | 3 | AUUAACACUUUGAAGAUAAU | 627 |
| T01_Cas14a.1 | 4 | GCCUGGGUGACAGUGAGACU | 628 |
| T02_Cas14a.1 | 4 | UUUUAAAAAGGUAUCUUUGA | 629 |
| T03_Cas14a.1 | 4 | ACGUGAUUUUCUGUUAAUAA | 630 |
| T04_Cas14a.1 | 4 | CAUUAAACAUCAUUAAAUUA | 631 |
| T05_Cas14a.1 | 4 | AUACUAACCUUGGUUUCUGU | 632 |
| T06_Cas14a.1 | 4 | AAAAGGUAUCUUUGAUACUA | 633 |
| T07_Cas14a.1 | 4 | UCAAAUGUAACCAGUAUUUU | 634 |
| T08_Cas14a.1 | 4 | AUGUAAAGUUAUUAACAGAA | 635 |
| T09_Cas14a.1 | 4 | UUUUCUUAGAGACAGAGUCU | 636 |
| T10_Cas14a.1 | 4 | UAUAUUUAUUUUCUUAGAGA | 637 |
| T11_Cas14a.1 | 4 | AAAUAAAAUACUGGUUACAU | 638 |
| T12_Cas14a.1 | 4 | AUGAUGUUUAAUGUAAAGUU | 639 |
| T13_Cas14a.1 | 4 | AUAAAAUUAUACCAUAGAUU | 640 |
| T14_Cas14a.1 | 4 | AUGGCAAAUAUUAGUUUCUG | 641 |
| T15_Cas14a.1 | 4 | AAUGUAACUUCCAAACGUUA | 642 |
| T16_Cas14a.1 | 4 | AAAAAUUAUUCAUUGUGUUA | 643 |
| T17_Cas14a.1 | 4 | UGUUGCUUAUUUAAAAAAUU | 644 |
| T18_Cas14a.1 | 4 | CUUUUUUGAUGGCAAAUAUU | 645 |
| T19_Cas14a.1 | 4 | ACAGAAACUAAUAUUUGCCA | 646 |
| T20_Cas14a.1 | 4 | AAAUGUCUCCUCCAGACUAG | 647 |
| T21_Cas14a.1 | 4 | CCAUCAAAAAAGCAAAGAAU | 648 |
| T22_Cas14a.1 | 4 | AAUAAGCAACAUAAAUGUGA | 649 |
| T23_Cas14a.1 | 4 | UUUUUCCUUUUAUUCUAGUU | 650 |
| T24_Cas14a.1 | 4 | UUCUAGUUGAAAGAAUUCAG | 651 |
| T25_Cas14a.1 | 4 | CUACUAUAUAUUUAUUUUUC | 652 |
| T26_Cas14a.1 | 4 | GAAGUUACAUUUAAAAUGUC | 653 |
| T27_Cas14a.1 | 4 | UAUCCUUGAUUAUACUUAGG | 654 |
| T28_Cas14a.1 | 4 | CAUUAAUUUAUAUCCUUGAU | 655 |
| T29_Cas14a.1 | 4 | AAGAAAAACUUCUGCCAACU | 656 |
| T30_Cas14a.1 | 4 | UCAUUUUUUCUCAUACCUUC | 657 |
| T31_Cas14a.1 | 4 | UUAUUUGCAUUAAUUUAUAU | 658 |
| T32_Cas14a.1 | 4 | CUGAGAGAGAAACAGUUGCC | 659 |
| T33_Cas14a.1 | 4 | CUCUCCUAGACCAUUUCCCA | 660 |
| T34_Cas14a.1 | 4 | AAAUGAAGAUUUUCCACCAA | 661 |
| T35_Cas14a.1 | 4 | AGCCAAACUCUUAUUCAUGA | 662 |
| T36_Cas14a.1 | 4 | GGUUUUUGCAAAAAGGAAAA | 663 |
| T37_Cas14a.1 | 4 | GCAAUACAUGGUAGAAAAUG | 664 |
| T38_Cas14a.1 | 4 | UUUAGCAAUACAUGGUAGAA | 665 |
| T39_Cas14a.1 | 4 | AUUGAAGAGUAACAAUUUGA | 666 |
| T40_Cas14a.1 | 4 | CAAAAAGGAAAAAAGAAGAA | 667 |
| T41_Cas14a.1 | 4 | GCUUGUGUUUCUAAUUUUUC | 668 |
| T42_Cas14a.1 | 4 | GCUCAAAUUGUUACUCUUCA | 669 |
| T43_Cas14a.1 | 4 | ACUUAUUGUUAUUGAAAUUG | 670 |
| T44_Cas14a.1 | 4 | GCUCCUACUCAGACUGUUAC | 671 |
| T45_Cas14a.1 | 4 | CAAAAACCCAAAAUAUUUUA | 672 |
| T46_Cas14a.1 | 4 | CACAGUCAAUAACACAAAGG | 673 |
| T47_Cas14a.1 | 4 | AACAAAUAGCUAGAGCCAAA | 674 |
| T48_Cas14a.1 | 4 | AAUCAGAGUCAAUUUCCAAG | 675 |
| T49_Cas14a.1 | 4 | UCUCUAUCUUUAGAAUUGAA | 676 |
| T50_Cas14a.1 | 4 | GAAUUGAAACAAAUUUUCUC | 677 |
| T51_Cas14a.1 | 4 | GCUCUAGCUAUUUGUUCAAA | 678 |
| T52_Cas14a.1 | 4 | UUUCAAUUCUAAAGAUAGAG | 679 |
| T53_Cas14a.1 | 4 | UUCAAAAGUGCAACUAUGAA | 680 |
| T54_Cas14a.1 | 4 | UGUUAUUGACUGUGCAAAAA | 681 |
| T55_Cas14a.1 | 4 | GUGGGUAGAAUUUCUUUUAA | 682 |
| T56_Cas14a.1 | 4 | GUGAAUAUAUUAUUGGAUUU | 683 |
| T57_Cas14a.1 | 4 | ACAGAAAAGCAUACACAUUA | 684 |
| T58_Cas14a.1 | 4 | CUUCUAAAUUAACUUUAGUG | 685 |
| T59_Cas14a.1 | 4 | AAGAUAAUUCAUGAACAUCU | 686 |
| T60_Cas14a.1 | 4 | GCCAACCACCCUACAAAUAU | 687 |
| T61_Cas14a.1 | 4 | AAGGAAUUAUAAUUAUUUUA | 688 |
| T62_Cas14a.1 | 4 | GAAGUAAAAUAUAAUAGAAA | 689 |
| T63_Cas14a.1 | 4 | UAGGGUGGUUGGCUAAAAUA | 690 |
| T64_Cas14a.1 | 4 | AAAGAAAUUCUACCCACUAA | 691 |
| T65_Cas14a.1 | 4 | CUGCUGUUGAUUAAUGGUUG | 692 |
| T66_Cas14a.1 | 4 | AUGUUAGUGCCUUUCACCCU | 693 |
| T67_Cas14a.1 | 4 | GUAUAUGAAAAAAAGAAAUA | 694 |
| T68_Cas14a.1 | 4 | AGCUUUCUUUAGAAGAAUAU | 695 |
| T69_Cas14a.1 | 4 | GAAGAAUAUUUCAUGAGAGA | 696 |
| T70_Cas14a.1 | 4 | GCAGACUUUUUAAGCUUUCU | 697 |
| T71_Cas14a.1 | 4 | UUUCUUUUUUUCAUAUACUA | 698 |
| T72_Cas14a.1 | 4 | AGAAAAGAUUAAACAGUGUG | 699 |
| T73_Cas14a.1 | 4 | UUUGAGAAAAGAUUAAACAG | 700 |
| T74_Cas14a.1 | 4 | UUUUGCCUUUUUGGUAUCUU | 701 |
| T75_Cas14a.1 | 4 | UAUUUCUUUCUUUGCCAGUA | 702 |
| T76_Cas14a.1 | 4 | GUAUCUUACAGGAACUCCAG | 703 |
| T77_Cas14a.1 | 4 | CCUUUUUGGUAUCUUACAGG | 704 |
| T78_Cas14a.1 | 4 | CCAGUACAACUGCAUGUGGU | 705 |
| T79_Cas14a.1 | 4 | GAUAUGGGCAUGUCAGUUUC | 706 |
| T80_Cas14a.1 | 4 | AUCUUUUCUCAAAUAAAAAG | 707 |
| T81_Cas14a.1 | 4 | AGAAGUUAAAGAGUCCAGAU | 708 |
| T82_Cas14a.1 | 4 | AUUUGUUUUUUCGAAAUUGU | 709 |
| T83_Cas14a.1 | 4 | ACUGUUGUUGUCAUCAUUAU | 710 |
| T84_Cas14a.1 | 4 | UUUUUUCGAAAUUGUAUUUA | 711 |
| T85_Cas14a.1 | 4 | ACUUCUUAAAGAUCAGGUUC | 712 |
| T86_Cas14a.1 | 4 | UCUUCAGCACAUCUGGACUC | 713 |
| T87_Cas14a.1 | 4 | CAGAGUUUAGUUUCAAGUAA | 714 |
| T88_Cas14a.1 | 4 | GUUUCAAGUAACAAAUAAAA | 715 |
| T89_Cas14a.1 | 4 | GUUAUACUGACAAAGAUAUC | 716 |
| T90_Cas14a.1 | 4 | UUUGUUACUUGAAACUAAAC | 717 |
| T91_Cas14a.1 | 4 | UUACUUGAAACUAAACUCUG | 718 |
| T92_Cas14a.1 | 4 | ACAGAUCUGUUGAGAAAUGG | 719 |
| T93_Cas14a.1 | 4 | CAUAAUCCAUCUAUUUUUCU | 720 |
| T94_Cas14a.1 | 4 | CCUGCAGGCGAUUUGACAGA | 721 |
| T95_Cas14a.1 | 4 | AAAUGUUGUGUGUACAUGCU | 722 |
| T96_Cas14a.1 | 4 | UCAGUAUAACCAAAAAAUAU | 723 |
| T97_Cas14a.1 | 4 | ACAUUUCAUUCAACUGUUGC | 724 |
| T98_Cas14a.1 | 4 | GAUUGCAUCUACUGUAUAGG | 725 |
| T99_Cas14a.1 | 4 | UAACUUGAUCAAGCAGAGAA | 726 |
| T100_Cas14a.1 | 4 | GAGAUGGCAGUUUCCUUAGU | 727 |
| T101_Cas14a.1 | 4 | CCCUCAGCUCUUGAAGUAAA | 728 |
| T102_Cas14a.1 | 4 | CCGCCUUCCACUCAGAGCUC | 729 |
| T103_Cas14a.1 | 4 | CUUCAAGAGCUGAGGGCAAA | 730 |
| T104_Cas14a.1 | 4 | CAGACCUCCUGCCACCGCAG | 731 |
| T105_Cas14a.1 | 4 | CCGCUGCCCAAUGCCAUCCU | 732 |
| T106_Cas14a.1 | 4 | AGGAUUGCUGAAUUAUUUCU | 733 |
| T107_Cas14a.1 | 4 | GCAUGUUCCCAAUUCUCAGG | 734 |
| T108_Cas14a.1 | 4 | UAUCAUAAUGAAAACGCCGC | 735 |
| T109_Cas14a.1 | 4 | UGUCUUUCUGAGAAACUGUU | 736 |
| T110_Cas14a.1 | 4 | UAUUUAGCAUGUUCCCAAUU | 737 |
| T111_Cas14a.1 | 4 | AUCAGUGGCUAACAGAAGCU | 738 |

TABLE 5 provides illustrative repeat sequences for use in guide nucleic acids that are useful in the compositions, systems and methods described herein.

TABLE 5
EXEMPLARY REPEAT SEQUENCES FOR USE IN GUIDE NUCLEIC ACIDS

| Effector Protein | Repeat Sequence (5′→3′), | Repeat Sequence |
|---|---|---|
| SEQ ID NO: | shown as RNA | SEQ ID NO: |
| 1 | CCUGAUUGCUCGCUGCGGCGAGAC | 739 |
| 2 | AUUGCUCCUUACGAGGAGAC | 740 |
| 2 | gAUUGCUCCUUACGAGGAGAC | 741 |
| 2, CasPhi.12 | AUAGAUUGCUCCUUACGAGGAGAC | 742 |
| (SEQ ID NO: 2) | | |
| L26R Variant | | |
| 3 | UGGUACAUCCAAC | 1634 |
| 4 | GAAUGAAGGAAUGCAAC | 745 |

TABLE 5.1 provides illustrative intermediary sequences for use in guide nucleic acids that are useful in the compositions, systems and methods described herein.

TABLE 5.1
EXEMPLARY INTERMEDIARY SEQUENCES FOR USE IN GUIDE NUCLEIC ACIDS

| Effector Protein | Intermediary Sequence (5′→3′), | Intermediary Sequence |
|---|---|---|
| SEQ ID NO: | shown as RNA | SEQ ID NO: |
| 3 | UGGGGCAGUUGGUUGCCCUUAGCCUGAGGCAUUUAUUGC | 1635 |
| | ACUCGGGAAGUACCAUUUCUCA | |
| 4 | ACCGCUUCACCAAgUGCUGUCCCUUAGGGAUUAGcACUU | 747 |
| | GAGUGAAGGUGGGCUGCUUGCAUCAGCCUAAUGUCGAGA | |
| | AGUGCUUUCUUCGGAAAGUAACCCUCGAAACAAAUUCAU | |
| | UU | |

TABLE 6 provides illustrative handle sequences for use in guide nucleic acids that are useful in the compositions, systems and methods described herein.

TABLE 6
EXEMPLARY HANDLE SEQUENCES FOR USE IN GUIDE NUCLEIC ACIDS

| Effector Protein | | Handle Sequence |
|---|---|---|
| SEQ ID NO: | Handle Sequence (5′→3′), shown as RNA | SEQ ID NO: |
| 3 | UGGGGCAGUUGGUUGCCCUUAGCCUGAGGCAUUUAUUGCACUCGGGA | 743 |
| | AGUACCAUUUCUCAGAAAUGGUACAUCCAAC | |
| 4 | ACCGCUUCACCAAGUGCUGUCCCUUAGGGGAUUAGCACUUGAGUGAA | 744 |
| | GGUGGGCUGCUUGCAUCAGCCUAAUGUCGAGAAGUGCUUUCUUCGGA | |
| | AAGUAACCCUCGAAACAAAUUCAUUUGAAAGAAUGAAGGAAUGCAAC | |
| 4 | ACCGCUUCACCAAgUGCUGUCCCUUAGGGAUUAGcACUUGAGUGAAG | 746 |
| | GUGGGCUGCUUGCAUCAGCCUAAUGUCGAGAAGUGCUUUCUUCGGAA | |
| | AGUAACCCUCGAAACAAAUUCAUUUGAAAGAAUGAAGGAAUGCAAC | |

TABLE 7 provides illustrative crRNA sequences that are useful in the compositions, systems and methods described herein, wherein.

TABLE 7
EXEMPLARY crRNA SEQUENCES FOR USE IN SINGLE GUIDE SYSTEMS

| Effector | | | | |
|---|---|---|---|---|
| Protein | | | | crRNA |
| SEQ ID | Composition | | | Sequence |
| NO: | Name | PAM | crRNA Sequence (5′→3′), shown as RNA | SEQ ID NO: |
| 1 | T01_CasPhi32 | GTTA | CCUGAUUGCUCGCUGCGGCGAGACUACUGACAAAGAUAUCACUCUUUUA | 748 |
| 1 | T02_CasPhi32 | GTTT | CCUGAUUGCUCGCUGCGGCGAGACCCUGCAUUUGCAGAGUUUAGUUUUA | 749 |
| 1 | T03_CasPhi32 | GTTA | CCUGAUUGCUCGCUGCGGCGAGACCUUGAAACUAAACUCUGCAAUUUUA | 750 |
| 1 | T04_CasPhi32 | GTTT | CCUGAUUGCUCGCUGCGGCGAGACUACAUAAUCCAUCUAUUUUUUUUUA | 751 |
| 1 | T05_CasPhi32 | GTTC | CCUGAUUGCUCGCUGCGGCGAGACUGAAGGGUGAUGGAAAUUACUUUUA | 752 |
| 1 | T06_CasPhi32 | GTTA | CCUGAUUGCUCGCUGCGGCGAGACAAGAGUCCAGAUGUGCUGAAUUUUA | 753 |
| 1 | T07_CasPhi32 | GTTG | CCUGAUUGCUCGCUGCGGCGAGACUCAUCAUUAUAUUACUAGAAUUUUA | 754 |
| 1 | T08_CasPhi32 | GTTT | CCUGAUUGCUCGCUGCGGCGAGACUUUCGAAAUUGUAUUUAUCUUUUUA | 755 |
| 1 | T09_CasPhi32 | GTTG | CCUGAUUGCUCGCUGCGGCGAGACUUGUCAUCAUUAUAUUACUAUUUUA | 756 |
| 1 | T10_CasPhi32 | GTTT | CCUGAUUGCUCGCUGCGGCGAGACCAUAGGGAAAUUUUCACAUGUUUUA | 757 |
| 1 | T11_CasPhi32 | GTTC | CCUGAUUGCUCGCUGCGGCGAGACCUGUAAGAUACCAAAAAGGCUUUUA | 758 |
| 1 | T12_CasPhi32 | GTTT | CCUGAUUGCUCGCUGCGGCGAGACUGCCUUUUUGGUAUCUUACAUUUUA | 759 |
| 1 | T13_CasPhi32 | GTTG | CCUGAUUGCUCGCUGCGGCGAGACUACUGGCAAAGAAAGAAAUAUUUUA | 760 |
| 1 | T14_CasPhi32 | GTTT | CCUGAUUGCUCGCUGCGGCGAGACAAUCUUUUCUCAAAUAAAAAUUUUA | 761 |
| 1 | T15_CasPhi32 | GTTG | CCUGAUUGCUCGCUGCGGCGAGACAUUAAUGGUUGAUAGGUUCUUUUUA | 762 |
| 1 | T16_CasPhi32 | GTTA | CCUGAUUGCUCGCUGCGGCGAGACGUGCCUUUCACCCUGCUUAUUUUUA | 763 |
| 1 | T17_CasPhi32 | GTTT | CCUGAUUGCUCGCUGCGGCGAGACUCAUUCCUAUUAGAUCUGUCUUUUA | 764 |
| 1 | T18_CasPhi32 | GTTG | CCUGAUUGCUCGCUGCGGCGAGACAUAGGUUCUUUAAUGUUAGUUUUUA | 765 |
| 1 | T19_CasPhi32 | GTTC | CCUGAUUGCUCGCUGCGGCGAGACUUUAAUGUUAGUGCCUUUCAUUUUA | 766 |
| 1 | T20_CasPhi32 | GTTA | CCUGAUUGCUCGCUGCGGCGAGACAUCGAAUAAGUAAUGUGUAUUUUUA | 767 |
| 1 | T21_CasPhi32 | GTTA | CCUGAUUGCUCGCUGCGGCGAGACGAAGAUCUGAGCUCUGAGUGUUUUA | 768 |
| 1 | T22_CasPhi32 | GTTA | CCUGAUUGCUCGCUGCGGCGAGACAAGAGGAAGUUAGAAGAUCUUUUUA | 769 |
| 1 | T23_CasPhi32 | GTTC | CCUGAUUGCUCGCUGCGGCGAGACAUGAAUUAUCUUCAAAGUGUUUUUA | 770 |
| 1 | T24_CasPhi32 | GTTA | CCUGAUUGCUCGCUGCGGCGAGACAUUUAGAAGUAAAAUAUAAUUUUUA | 771 |
| 1 | T25_CasPhi32 | GTTC | CCUGAUUGCUCGCUGCGGCGAGACAAAAGUGCAACUAUGAAGUGUUUUA | 772 |
| 1 | T26_CasPhi32 | GTTT | CCUGAUUGCUCGCUGCGGCGAGACAUCUCUAUCUUUAGAAUUGAUUUUA | 773 |
| 1 | T27_CasPhi32 | GTTT | CCUGAUUGCUCGCUGCGGCGAGACCAAUUCUAAAGAUAGAGAUAUUUUA | 774 |
| 1 | T28_CasPhi32 | GTTG | CCUGAUUGCUCGCUGCGGCGAGACCACUUUUGAACAAAUAGCUAUUUUA | 775 |
| 1 | T29_CasPhi32 | GTTA | CCUGAUUGCUCGCUGCGGCGAGACUUGACUGUGCAAAAAGUCUUUUUUA | 776 |
| 1 | T30_CasPhi32 | GTTT | CCUGAUUGCUCGCUGCGGCGAGACGGCUCAAAUUGUUACUCUUCUUUUA | 777 |
| 1 | T31_CasPhi32 | GTTA | CCUGAUUGCUCGCUGCGGCGAGACUUGAAAUUGGCUCUUUAGCUUUUUA | 778 |
| 1 | T32_CasPhi32 | GTTA | CCUGAUUGCUCGCUGCGGCGAGACCUCUUCAAUUAAAUUUGACUUUUUA | 779 |
| 1 | T33_CasPhi32 | GTTT | CCUGAUUGCUCGCUGCGGCGAGACUUGCAAAAAGGAAAAAAGAAUUUUA | 780 |
| 1 | T34_CasPhi32 | GTTT | CCUGAUUGCUCGCUGCGGCGAGACCUAAUUUUUCUUUUUCUUCUUUUUA | 781 |
| 1 | T35_CasPhi32 | GTTT | CCUGAUUGCUCGCUGCGGCGAGACGCUGAGAGAGAAACAGUUGCUUUUA | 782 |
| 1 | T36_CasPhi32 | GTTT | CCUGAUUGCUCGCUGCGGCGAGACCUCUCUCAGCAAACACAUUAUUUUA | 783 |
| 1 | T37_CasPhi32 | GTTG | CCUGAUUGCUCGCUGCGGCGAGACCCUAAGAACUGGUGGGAAAUUUUUA | 784 |
| 1 | T38_CasPhi32 | GTTC | CCUGAUUGCUCGCUGCGGCGAGACUUAGGCAACUGUUUCUCUCUUUUUA | 785 |
| 1 | T39_CasPhi32 | GTTG | CCUGAUUGCUCGCUGCGGCGAGACGCAGAAGUUUUUCUUUAAAAUUUUA | 786 |
| 1 | T40_CasPhi32 | GTTT | CCUGAUUGCUCGCUGCGGCGAGACUUCUUUAAAAUGAAGAUUUUUUUUA | 787 |
| 1 | T41_CasPhi32 | GTTC | CCUGAUUGCUCGCUGCGGCGAGACAUCAUCCUAGCCAUAACACAUUUUA | 788 |
| 1 | T42_CasPhi32 | GTTG | CCUGAUUGCUCGCUGCGGCGAGACUUCAUCAUCCUAGCCAUAACUUUUA | 789 |
| 1 | T43_CasPhi32 | GTTG | CCUGAUUGCUCGCUGCGGCGAGACAAAGAAUUCAGAAUCAGUGGUUUUA | 790 |
| 1 | T44_CasPhi32 | GTTT | CCUGAUUGCUCGCUGCGGCGAGACGGAAGUUACAUUUAAAAUGUUUUUA | 791 |
| 1 | T45_CasPhi32 | GTTA | CCUGAUUGCUCGCUGCGGCGAGACCAUUUAAAAUGUCUCCUCCAUUUUA | 792 |
| 1 | T46_CasPhi32 | GTTA | CCUGAUUGCUCGCUGCGGCGAGACUCUCACAUUUAUGUUGCUUAUUUUA | 793 |
| 1 | T47_CasPhi32 | GTTG | CCUGAUUGCUCGCUGCGGCGAGACCUUAUUUAAAAAAUUAUUCAUUUUA | 794 |
| 1 | T48_CasPhi32 | GTTA | CCUGAUUGCUCGCUGCGGCGAGACUUAACAGAAAAUCACGUUAAUUUUA | 795 |
| 1 | T49_CasPhi32 | GTTA | CCUGAUUGCUCGCUGCGGCGAGACAAGCUGAAAUGAACAGUAGAUUUUA | 796 |
| 1 | T50_CasPhi32 | GTTC | CCUGAUUGCUCGCUGCGGCGAGACAUUUCAGCUUUAACGUGAUUUUUUA | 797 |
| 1 | T51_CasPhi32 | GTTA | CCUGAUUGCUCGCUGCGGCGAGACAUAACUUUACAUUAAACAUCUUUUA | 798 |
| 1 | T52_CasPhi32 | GTTA | CCUGAUUGCUCGCUGCGGCGAGACGUAUCAAAGAUACCUUUUUAUUUUA | 799 |
| 1 | T53_CasPhi32 | GTTA | CCUGAUUGCUCGCUGCGGCGAGACCAUUUGAUAAAAUUAUACCAUUUUA | 800 |
| 1 | T54_CasPhi32 | GTTT | CCUGAUUGCUCGCUGCGGCGAGACAAUGUAAAGUUAUUAACAGAUUUUA | 801 |
| 1 | T55_CasPhi32 | GTTT | CCUGAUUGCUCGCUGCGGCGAGACCUGUGAUUUUCUUUUGGAUUUUUUA | 802 |
| 1 | T56_CasPhi32 | GTTN | CCUGAUUGCUCGCUGCGGCGAGACAAUCCUUUAACAUUUCAUUC | 803 |
| 1 | T57_CasPhi32 | GTTN | CCUGAUUGCUCGCUGCGGCGAGACUGAAGGUGUUCUUGUACUUC | 804 |
| 1 | T58_CasPhi32 | GTTN | CCUGAUUGCUCGCUGCGGCGAGACUUGUACUUCAUCCCACUGAU | 805 |
| 1 | T59_CasPhi32 | GTTN | CCUGAUUGCUCGCUGCGGCGAGACCCUCCGGUUCUGAAGGUGUU | 806 |
| 1 | T60_CasPhi32 | GTTN | CCUGAUUGCUCGCUGCGGCGAGACAAGGAUUCAACACAAUGGCU | 807 |
| 1 | T61_CasPhi32 | GTTN | CCUGAUUGCUCGCUGCGGCGAGACAAUGAAAUGUUAAAGGAUUC | 808 |
| 1 | T62_CasPhi32 | GTTN | CCUGAUUGCUCGCUGCGGCGAGACUGUCACCAGAGUAACAGUCU | 809 |
| 1 | T63_CasPhi32 | GTTN | CCUGAUUGCUCGCUGCGGCGAGACUGUCCAAGCCCGGUUGAAAU | 810 |
| 1 | T64_CasPhi32 | GTTN | CCUGAUUGCUCGCUGCGGCGAGACGGAGAUGGCAGUUUCCUUAG | 811 |
| 1 | T65_CasPhi32 | GTTN | CCUGAUUGCUCGCUGCGGCGAGACCCUUAGUAACCACAGGUUGU | 812 |
| 1 | T66_CasPhi32 | GTTN | CCUGAUUGCUCGCUGCGGCGAGACAAAUCUGCCAGAGCAGGUAC | 813 |
| 1 | T67_CasPhi32 | GTTN | CCUGAUUGCUCGCUGCGGCGAGACAUAUCCUCAAGGUCACCCAC | 814 |
| 1 | T68_CasPhi32 | GTTN | CCUGAUUGCUCGCUGCGGCGAGACUAAAAUCACAGAGGGUGAUG | 815 |
| 1 | T69_CasPhi32 | GTTN | CCUGAUUGCUCGCUGCGGCGAGACGAGGUACCUGCUCUGGCAGA | 816 |
| 1 | T70_CasPhi32 | GTTN | CCUGAUUGCUCGCUGCGGCGAGACCUCUGGUGACACAACCUGUG | 817 |
| 1 | T71_CasPhi32 | GTTN | CCUGAUUGCUCGCUGCGGCGAGACCUAAGGAAACUGCCAUCUCC | 818 |
| 1 | T72_CasPhi32 | GTTN | CCUGAUUGCUCGCUGCGGCGAGACACCGCCUUCCACUCAGAGCU | 819 |
| 1 | T73_CasPhi32 | GTTN | CCUGAUUGCUCGCUGCGGCGAGACACUUCAAGAGCUGAGGGCAA | 820 |
| 1 | T74_CasPhi32 | GTTN | CCUGAUUGCUCGCUGCGGCGAGACUUGAGGAUUGCUGAAUUAUU | 821 |
| 1 | T75_CasPhi32 | GTTN | CCUGAUUGCUCGCUGCGGCGAGACGCAGACCUCCUGCCACCGCA | 822 |
| 1 | T76_CasPhi32 | GTTN | CCUGAUUGCUCGCUGCGGCGAGACCAUUCAAUGUUCUGACAACA | 823 |
| 1 | T77_CasPhi32 | GTTN | CCUGAUUGCUCGCUGCGGCGAGACUGACAACAGUUUGCCGCUGC | 824 |
| 1 | T78_CasPhi32 | GTTN | CCUGAUUGCUCGCUGCGGCGAGACGCCGCUGCCCAAUGCCAUCC | 825 |
| 1 | T79_CasPhi32 | GTTN | CCUGAUUGCUCGCUGCGGCGAGACUCAGAACAUUGAAUGCAACU | 826 |
| 1 | T80_CasPhi32 | GTTN | CCUGAUUGCUCGCUGCGGCGAGACCCAAUUCUCAGGAAUUUGUG | 827 |
| 1 | T81_CasPhi32 | GTTN | CCUGAUUGCUCGCUGCGGCGAGACAGCUUCUGUUAGCCACUGAU | 828 |
| 1 | T82_CasPhi32 | GTTN | CCUGAUUGCUCGCUGCGGCGAGACGCCACUGAUUAAAUAUCUUU | 829 |
| 1 | T83_CasPhi32 | GTTN | CCUGAUUGCUCGCUGCGGCGAGACCUCAGAAAGACACAAAUUCC | 830 |
| 1 | T84_CasPhi32 | GTTN | CCUGAUUGCUCGCUGCGGCGAGACAGAAAUGGCGGCGUUUUCAU | 831 |
| 2 | T001_CasPhi.12 | TTTA | GAUUGCUCCUUACGAGGAGACCCUGCAGGCGAUUUGACUUUUA | 832 |
| 2 | T002_CasPhi.12 | TTTT | GAUUGCUCCUUACGAGGAGACACCUGCAGGCGAUUUGAUUUUA | 833 |
| 2 | T003_CasPhi.12 | CTTT | GAUUGCUCCUUACGAGGAGACUACCUGCAGGCGAUUUGUUUUA | 834 |
| 2 | T004_CasPhi.12 | ATTA | GAUUGCUCCUUACGAGGAGACUAGAGAUAUAGCGUAUAUUUUA | 835 |
| 2 | T005_CasPhi.12 | TTTT | GAUUGCUCCUUACGAGGAGACGGUUAUACUGACAAAGAUUUUA | 836 |
| 2 | T006_CasPhi.12 | ATTT | GAUUGCUCCUUACGAGGAGACGACAGAUCUGUUGAGAAUUUUA | 837 |
| 2 | T007_CasPhi.12 | ATTT | GAUUGCUCCUUACGAGGAGACCUCAACAGAUCUGUCAAUUUUA | 838 |
| 2 | T008_CasPhi.12 | CTTG | GAUUGCUCCUUACGAGGAGACAUCCAUAUGCUUUUACCUUUUA | 839 |
| 2 | T009_CasPhi.12 | ATTT | GAUUGCUCCUUACGAGGAGACGUUACUUGAAACUAAACUUUUA | 840 |
| 2 | T010_CasPhi.12 | ATTT | GAUUGCUCCUUACGAGGAGACGCAGAGUUUAGUUUCAAUUUUA | 841 |
| 2 | T011_CasPhi.12 | TTTG | GAUUGCUCCUUACGAGGAGACGUUAUACUGACAAAGAUUUUUA | 842 |
| 2 | T012_CasPhi.12 | ATTT | GAUUGCUCCUUACGAGGAGACUUUGGUUAUACUGACAAUUUUA | 843 |
| 2 | T013_CasPhi.12 | GTTT | GAUUGCUCCUUACGAGGAGACCCUGCAUUUGCAGAGUUUUUUA | 844 |
| 2 | T014_CasPhi.12 | GTTA | GAUUGCUCCUUACGAGGAGACCUUGAAACUAAACUCUGUUUUA | 845 |
| 2 | T015_CasPhi.12 | ATTT | GAUUGCUCCUUACGAGGAGACUUCUUGAUCCAUAUGCUUUUUA | 846 |
| 2 | T016_CasPhi.12 | TTTT | GAUUGCUCCUUACGAGGAGACCUUGAUCCAUAUGCUUUUUUUA | 847 |
| 2 | T017_CasPhi.12 | TTTT | GAUUGCUCCUUACGAGGAGACUCUUGAUCCAUAUGCUUUUUUA | 848 |
| 2 | T018_CasPhi.12 | GTTA | GAUUGCUCCUUACGAGGAGACUACUGACAAAGAUAUCAUUUUA | 849 |
| 2 | T019_CasPhi.12 | CTTT | GAUUGCUCCUUACGAGGAGACGUCAGUAUAACCAAAAAUUUUA | 850 |
| 2 | T020_CasPhi.12 | TTTC | GAUUGCUCCUUACGAGGAGACCUGCAUUUGCAGAGUUUUUUUA | 851 |
| 2 | T021_CasPhi.12 | GTTT | GAUUGCUCCUUACGAGGAGACUACAUAAUCCAUCUAUUUUUUA | 852 |
| 2 | T022_CasPhi.12 | CTTG | GAUUGCUCCUUACGAGGAGACAAACUAAACUCUGCAAAUUUUA | 853 |
| 2 | T023_CasPhi.12 | TTTG | GAUUGCUCCUUACGAGGAGACACAGAUCUGUUGAGAAAUUUUA | 854 |
| 2 | T024_CasPhi.12 | TTTA | GAUUGCUCCUUACGAGGAGACGUUUCAAGUAACAAAUAUUUUA | 855 |
| 2 | T025_CasPhi.12 | ATTA | GAUUGCUCCUUACGAGGAGACUGUAAAACAGAUUAUAGUUUUA | 856 |
| 2 | T026_CasPhi.12 | TTTG | GAUUGCUCCUUACGAGGAGACUUACUUGAAACUAAACUUUUUA | 857 |
| 2 | T027_CasPhi.12 | TTTT | GAUUGCUCCUUACGAGGAGACACAUAAUCCAUCUAUUUUUUUA | 858 |
| 2 | T028_CasPhi.12 | TTTA | GAUUGCUCCUUACGAGGAGACCAUAAUCCAUCUAUUUUUUUUA | 859 |
| 2 | T029_CasPhi.12 | TTTG | GAUUGCUCCUUACGAGGAGACUCAGUAUAACCAAAAAAUUUUA | 860 |
| 2 | T030_CasPhi.12 | TTTC | GAUUGCUCCUUACGAGGAGACUUGAUCCAUAUGCUUUUUUUUA | 861 |
| 2 | T031_CasPhi.12 | TTTC | GAUUGCUCCUUACGAGGAGACCAUCACCCUUCAGAACCUUUUA | 862 |
| 2 | T032_CasPhi.12 | CTTC | GAUUGCUCCUUACGAGGAGACAGCACAUCUGGACUCUUUUUUA | 863 |
| 2 | T033_CasPhi.12 | GTTA | GAUUGCUCCUUACGAGGAGACAAGAGUCCAGAUGUGCUUUUUA | 864 |
| 2 | T034_CasPhi.12 | ATTT | GAUUGCUCCUUACGAGGAGACAUCUUCAGCACAUCUGGUUUUA | 865 |
| 2 | T035_CasPhi.12 | TTTT | GAUUGCUCCUUACGAGGAGACGACUGUUGUUGUCAUCAUUUUA | 866 |
| 2 | T036_CasPhi.12 | CTTA | GAUUGCUCCUUACGAGGAGACAAGAUCAGGUUCUGAAGUUUUA | 867 |
| 2 | T037_CasPhi.12 | TTTA | GAUUGCUCCUUACGAGGAGACAGAAGUUAAAGAGUCCAUUUUA | 868 |
| 2 | T038_CasPhi.12 | CTTT | GAUUGCUCCUUACGAGGAGACAAGAAGUUAAAGAGUCCUUUUA | 869 |
| 2 | T039_CasPhi.12 | CTTA | GAUUGCUCCUUACGAGGAGACCCUUAAGAUACCAUUUGUUUUA | 870 |
| 2 | T040_CasPhi.12 | TTTA | GAUUGCUCCUUACGAGGAGACUCUUCAGCACAUCUGGAUUUUA | 871 |
| 2 | T041_CasPhi.12 | GTTC | GAUUGCUCCUUACGAGGAGACUGAAGGGUGAUGGAAAUUUUUA | 872 |
| 2 | T042_CasPhi.12 | TTTA | GAUUGCUCCUUACGAGGAGACACUUCUUAAAGAUCAGGUUUUA | 873 |
| 2 | T043_CasPhi.12 | CTTT | GAUUGCUCCUUACGAGGAGACUGACUGUUGUUGUCAUCUUUUA | 874 |
| 2 | T044_CasPhi.12 | TTTG | GAUUGCUCCUUACGAGGAGACACUGUUGUUGUCAUCAUUUUUA | 875 |
| 2 | T045_CasPhi.12 | TTTT | GAUUGCUCCUUACGAGGAGACUCGAAAUUGUAUUUAUCUUUUA | 876 |
| 2 | T046_CasPhi.12 | CTTT | GAUUGCUCCUUACGAGGAGACCUAGUAAUAUAAUGAUGUUUUA | 877 |
| 2 | T047_CasPhi.12 | CTTA | GAUUGCUCCUUACGAGGAGACAGGUAAGUCUUUGAUUUUUUUA | 878 |
| 2 | T048_CasPhi.12 | CTTC | GAUUGCUCCUUACGAGGAGACUUAAAGAUCAGGUUCUGUUUUA | 879 |
| 2 | T049_CasPhi.12 | TTTT | GAUUGCUCCUUACGAGGAGACCGAAAUUGUAUUUAUCUUUUUA | 880 |
| 2 | T050_CasPhi.12 | CTTT | GAUUGCUCCUUACGAGGAGACAACUUCUUAAAGAUCAGUUUUA | 881 |
| 2 | T051_CasPhi.12 | ATTT | GAUUGCUCCUUACGAGGAGACGUUUUUUCGAAAUUGUAUUUUA | 882 |
| 2 | T052_CasPhi.12 | ATTG | GAUUGCUCCUUACGAGGAGACUAUUUAUCUUCAGCACAUUUUA | 883 |
| 2 | T053_CasPhi.12 | TTTC | GAUUGCUCCUUACGAGGAGACUAGUAAUAUAAUGAUGAUUUUA | 884 |
| 2 | T054_CasPhi.12 | GTTG | GAUUGCUCCUUACGAGGAGACUCAUCAUUAUAUUACUAUUUUA | 885 |
| 2 | T055_CasPhi.12 | CTTA | GAUUGCUCCUUACGAGGAGACAGAUACCAUUUGUAUUUUUUUA | 886 |
| 2 | T056_CasPhi.12 | ATTA | GAUUGCUCCUUACGAGGAGACCUAGAAAGAAAAUUAUCUUUUA | 887 |
| 2 | T057_CasPhi.12 | TTTC | GAUUGCUCCUUACGAGGAGACGAAAUUGUAUUUAUCUUUUUUA | 888 |
| 2 | T058_CasPhi.12 | ATTT | GAUUGCUCCUUACGAGGAGACUCUUUCUAGUAAUAUAAUUUUA | 889 |
| 2 | T059_CasPhi.12 | TTTT | GAUUGCUCCUUACGAGGAGACUUCGAAAUUGUAUUUAUUUUUA | 890 |
| 2 | T060_CasPhi.12 | GTTT | GAUUGCUCCUUACGAGGAGACUUUCGAAAUUGUAUUUAUUUUA | 891 |
| 2 | T061_CasPhi.12 | GTTG | GAUUGCUCCUUACGAGGAGACUUGUCAUCAUUAUAUUAUUUUA | 892 |
| 2 | T062_CasPhi.12 | TTTC | GAUUGCUCCUUACGAGGAGACUUUCUAGUAAUAUAAUGUUUUA | 893 |
| 2 | T063_CasPhi.12 | ATTA | GAUUGCUCCUUACGAGGAGACUAUUACUAGAAAGAAAAUUUUA | 894 |
| 2 | T064_CasPhi.12 | CTTT | GAUUGCUCCUUACGAGGAGACGAUUUGUUUUUUCGAAAUUUUA | 895 |
| 2 | T065_CasPhi.12 | TTTG | GAUUGCUCCUUACGAGGAGACUUUUUUCGAAAUUGUAUUUUUA | 896 |
| 2 | T066_CasPhi.12 | ATTA | GAUUGCUCCUUACGAGGAGACCUUUUGACUGUUGUUGUUUUUA | 897 |
| 2 | T067_CasPhi.12 | TTTG | GAUUGCUCCUUACGAGGAGACAUUUGUUUUUUCGAAAUUUUUA | 898 |
| 2 | T068_CasPhi.12 | CTTT | GAUUGCUCCUUACGAGGAGACGCCAGUACAACUGCAUGUUUUA | 899 |
| 2 | T069_CasPhi.12 | ATTA | GAUUGCUCCUUACGAGGAGACAACAGUGUGCUACCACAUUUUA | 900 |
| 2 | T070_CasPhi.12 | TTTT | GAUUGCUCCUUACGAGGAGACGGUAUCUUACAGGAACUUUUUA | 901 |
| 2 | T071_CasPhi.12 | TTTT | GAUUGCUCCUUACGAGGAGACUGGUAUCUUACAGGAACUUUUA | 902 |
| 2 | T072_CasPhi.12 | TTTG | GAUUGCUCCUUACGAGGAGACGUAUCUUACAGGAACUCUUUUA | 903 |
| 2 | T073_CasPhi.12 | TTTG | GAUUGCUCCUUACGAGGAGACCCAGUACAACUGCAUGUUUUUA | 904 |
| 2 | T074_CasPhi.12 | TTTC | GAUUGCUCCUUACGAGGAGACUUUGCCAGUACAACUGCUUUUA | 905 |
| 2 | T075_CasPhi.12 | CTTA | GAUUGCUCCUUACGAGGAGACCAGGAACUCCAGGAUGGUUUUA | 906 |
| 2 | T076_CasPhi.12 | CTTT | GAUUGCUCCUUACGAGGAGACCUUUGCCAGUACAACUGUUUUA | 907 |
| 2 | T077_CasPhi.12 | TTTT | GAUUGCUCCUUACGAGGAGACCACAUGGAGCUUUUGUAUUUUA | 908 |
| 2 | T078_CasPhi.12 | ATTT | GAUUGCUCCUUACGAGGAGACCCCUAUGAAACUGACAUUUUUA | 909 |
| 2 | T079_CasPhi.12 | TTTC | GAUUGCUCCUUACGAGGAGACCCUAUGAAACUGACAUGUUUUA | 910 |
| 2 | T080_CasPhi.12 | CTTT | GAUUGCUCCUUACGAGGAGACUUGGUAUCUUACAGGAAUUUUA | 911 |
| 2 | T081_CasPhi.12 | TTTC | GAUUGCUCCUUACGAGGAGACACAUGGAGCUUUUGUAUUUUUA | 912 |
| 2 | T082_CasPhi.12 | GTTT | GAUUGCUCCUUACGAGGAGACCAUAGGGAAAUUUUCACUUUUA | 913 |
| 2 | T083_CasPhi.12 | ATTT | GAUUGCUCCUUACGAGGAGACUCACAUGGAGCUUUUGUUUUUA | 914 |
| 2 | T084_CasPhi.12 | TTTT | GAUUGCUCCUUACGAGGAGACGCCUUUUUGGUAUCUUAUUUUA | 915 |
| 2 | T085_CasPhi.12 | TTTG | GAUUGCUCCUUACGAGGAGACCCUUUUUGGUAUCUUACUUUUA | 916 |
| 2 | T086_CasPhi.12 | ATTT | GAUUGCUCCUUACGAGGAGACCUUUCUUUGCCAGUACAUUUUA | 917 |
| 2 | T087_CasPhi.12 | TTTC | GAUUGCUCCUUACGAGGAGACAUAGGGAAAUUUUCACAUUUUA | 918 |
| 2 | T088_CasPhi.12 | GTTG | GAUUGCUCCUUACGAGGAGACUACUGGCAAAGAAAGAAUUUUA | 919 |
| 2 | T089_CasPhi.12 | TTTC | GAUUGCUCCUUACGAGGAGACUUUCUUUGCCAGUACAAUUUUA | 920 |
| 2 | T090_CasPhi.12 | ATTT | GAUUGCUCCUUACGAGGAGACGAGAAAAGAUUAAACAGUUUUA | 921 |
| 2 | T091_CasPhi.12 | GTTT | GAUUGCUCCUUACGAGGAGACUGCCUUUUUGGUAUCUUUUUUA | 922 |
| 2 | T092_CasPhi.12 | TTTT | GAUUGCUCCUUACGAGGAGACGUUUUGCCUUUUUGGUAUUUUA | 923 |
| 2 | T093_CasPhi.12 | TTTT | GAUUGCUCCUUACGAGGAGACCUCAAAUAAAAAGACAUUUUUA | 924 |
| 2 | T094_CasPhi.12 | TTTG | GAUUGCUCCUUACGAGGAGACAGAAAAGAUUAAACAGUUUUUA | 925 |
| 2 | T095_CasPhi.12 | GTTT | GAUUGCUCCUUACGAGGAGACAAUCUUUUCUCAAAUAAUUUUA | 926 |
| 2 | T096_CasPhi.12 | TTTT | GAUUGCUCCUUACGAGGAGACUAUUUGAGAAAAGAUUAUUUUA | 927 |
| 2 | T097_CasPhi.12 | TTTT | GAUUGCUCCUUACGAGGAGACGUAUUUCUUUCUUUGCCUUUUA | 928 |
| 2 | T098_CasPhi.12 | TTTT | GAUUGCUCCUUACGAGGAGACAUUUGAGAAAAGAUUAAUUUUA | 929 |
| 2 | T099_CasPhi.12 | TTTC | GAUUGCUCCUUACGAGGAGACUCAAAUAAAAAGACAUGUUUUA | 930 |
| 2 | T100_CasPhi.12 | TTTA | GAUUGCUCCUUACGAGGAGACAUCUUUUCUCAAAUAAAUUUUA | 931 |
| 2 | T101_CasPhi.12 | CTTT | GAUUGCUCCUUACGAGGAGACUUAUUUGAGAAAAGAUUUUUUA | 932 |
| 2 | T102_CasPhi.12 | TTTG | GAUUGCUCCUUACGAGGAGACUUUUGCCUUUUUGGUAUUUUUA | 933 |
| 2 | T103_CasPhi.12 | TTTG | GAUUGCUCCUUACGAGGAGACUAUUUCUUUCUUUGCCAUUUUA | 934 |
| 2 | T104_CasPhi.12 | ATTT | GAUUGCUCCUUACGAGGAGACUUGUUUUGCCUUUUUGGUUUUA | 935 |
| 2 | T105_CasPhi.12 | CTTT | GAUUGCUCCUUACGAGGAGACUCUCAAAUAAAAAGACAUUUUA | 936 |
| 2 | T106_CasPhi.12 | TTTT | GAUUGCUCCUUACGAGGAGACUGUUUUGCCUUUUUGGUUUUUA | 937 |
| 2 | T107_CasPhi.12 | CTTT | GAUUGCUCCUUACGAGGAGACUGUAUUUCUUUCUUUGCUUUUA | 938 |
| 2 | T108_CasPhi.12 | CTTC | GAUUGCUCCUUACGAGGAGACAUUUUUGUUUUGCCUUUUUUUA | 939 |
| 2 | T109_CasPhi.12 | ATTC | GAUUGCUCCUUACGAGGAGACCUAUUAGAUCUGUCGCCUUUUA | 940 |
| 2 | T110_CasPhi.12 | ATTA | GAUUGCUCCUUACGAGGAGACGAUCUGUCGCCCUACCUUUUUA | 941 |
| 2 | T111_CasPhi.12 | TTTC | GAUUGCUCCUUACGAGGAGACACCCUGCUUAUAAUCUCUUUUA | 942 |
| 2 | T112_CasPhi.12 | ATTA | GAUUGCUCCUUACGAGGAGACAAGAACCUAUCAACCAUUUUUA | 943 |
| 2 | T113_CasPhi.12 | GTTA | GAUUGCUCCUUACGAGGAGACGUGCCUUUCACCCUGCUUUUUA | 944 |
| 2 | T114_CasPhi.12 | ATTA | GAUUGCUCCUUACGAGGAGACUAAGCAGGGUGAAAGGCUUUUA | 945 |
| 2 | T115_CasPhi.12 | CTTT | GAUUGCUCCUUACGAGGAGACCACCCUGCUUAUAAUCUUUUUA | 946 |
| 2 | T116_CasPhi.12 | ATTT | GAUUGCUCCUUACGAGGAGACCAUGAGAGAUUAUAAGCUUUUA | 947 |
| 2 | T117_CasPhi.12 | ATTT | GAUUGCUCCUUACGAGGAGACUAGCAGACUUUUUAAGCUUUUA | 948 |
| 2 | T118_CasPhi.12 | GTTG | GAUUGCUCCUUACGAGGAGACAUUAAUGGUUGAUAGGUUUUUA | 949 |
| 2 | T119_CasPhi.12 | TTTA | GAUUGCUCCUUACGAGGAGACAUGUUAGUGCCUUUCACUUUUA | 950 |
| 2 | T120_CasPhi.12 | CTTT | GAUUGCUCCUUACGAGGAGACACUGCUGUUGAUUAAUGUUUUA | 951 |
| 2 | T121_CasPhi.12 | TTTA | GAUUGCUCCUUACGAGGAGACCUGCUGUUGAUUAAUGGUUUUA | 952 |
| 2 | T122_CasPhi.12 | TTTC | GAUUGCUCCUUACGAGGAGACUGUCUGACAGCUGUUUGUUUUA | 953 |
| 2 | T123_CasPhi.12 | TTTT | GAUUGCUCCUUACGAGGAGACAGCAGACUUUUUAAGCUUUUUA | 954 |
| 2 | T124_CasPhi.12 | ATTA | GAUUGCUCCUUACGAGGAGACAUCAACAGCAGUAAAGAUUUUA | 955 |
| 2 | T125_CasPhi.12 | TTTT | GAUUGCUCCUUACGAGGAGACCAUUCCUAUUAGAUCUGUUUUA | 956 |
| 2 | T126_CasPhi.12 | TTTT | GAUUGCUCCUUACGAGGAGACUUCUGUCUGACAGCUGUUUUUA | 957 |
| 2 | T127_CasPhi.12 | TTTC | GAUUGCUCCUUACGAGGAGACAUGAGAGAUUAUAAGCAUUUUA | 958 |
| 2 | T128_CasPhi.12 | TTTT | GAUUGCUCCUUACGAGGAGACUCUGUCUGACAGCUGUUUUUUA | 959 |
| 2 | T129_CasPhi.12 | TTTA | GAUUGCUCCUUACGAGGAGACGCAGACUUUUUAAGCUUUUUUA | 960 |
| 2 | T130_CasPhi.12 | ATTA | GAUUGCUCCUUACGAGGAGACAUGGUUGAUAGGUUCUUUUUUA | 961 |
| 2 | T131_CasPhi.12 | TTTT | GAUUGCUCCUUACGAGGAGACCUGUCUGACAGCUGUUUUUUUA | 962 |
| 2 | T132_CasPhi.12 | CTTT | GAUUGCUCCUUACGAGGAGACAAUGUUAGUGCCUUUCAUUUUA | 963 |
| 2 | T133_CasPhi.12 | TTTC | GAUUGCUCCUUACGAGGAGACAUUCCUAUUAGAUCUGUUUUUA | 964 |
| 2 | T134_CasPhi.12 | CTTT | GAUUGCUCCUUACGAGGAGACUUUCUGUCUGACAGCUGUUUUA | 965 |
| 2 | T135_CasPhi.12 | TTTA | GAUUGCUCCUUACGAGGAGACGAAGAAUAUUUCAUGAGUUUUA | 966 |
| 2 | T136_CasPhi.12 | GTTC | GAUUGCUCCUUACGAGGAGACUUUAAUGUUAGUGCCUUUUUUA | 967 |
| 2 | T137_CasPhi.12 | CTTT | GAUUGCUCCUUACGAGGAGACAGAAGAAUAUUUCAUGAUUUUA | 968 |
| 2 | T138_CasPhi.12 | GTTT | GAUUGCUCCUUACGAGGAGACUCAUUCCUAUUAGAUCUUUUUA | 969 |
| 2 | T139_CasPhi.12 | TTTC | GAUUGCUCCUUACGAGGAGACUUUACUGCUGUUGAUUAUUUUA | 970 |
| 2 | T140_CasPhi.12 | TTTT | GAUUGCUCCUUACGAGGAGACUAAGCUUUCUUUAGAAGUUUUA | 971 |
| 2 | T141_CasPhi.12 | TTTA | GAUUGCUCCUUACGAGGAGACAGCUUUCUUUAGAAGAAUUUUA | 972 |
| 2 | T142_CasPhi.12 | ATTT | GAUUGCUCCUUACGAGGAGACCUUUACUGCUGUUGAUUUUUUA | 973 |
| 2 | T143_CasPhi.12 | ATTC | GAUUGCUCCUUACGAGGAGACUUCUAAAGAAAGCUUAAUUUUA | 974 |
| 2 | T144_CasPhi.12 | GTTG | GAUUGCUCCUUACGAGGAGACAUAGGUUCUUUAAUGUUUUUUA | 975 |
| 2 | T145_CasPhi.12 | CTTA | GAUUGCUCCUUACGAGGAGACAAAAGUCUGCUAAAAUGUUUUA | 976 |
| 2 | T146_CasPhi.12 | TTTT | GAUUGCUCCUUACGAGGAGACAAGCUUUCUUUAGAAGAUUUUA | 977 |
| 2 | T147_CasPhi.12 | CTTT | GAUUGCUCCUUACGAGGAGACUUAAGCUUUCUUUAGAAUUUUA | 978 |
| 2 | T148_CasPhi.12 | CTTA | GAUUGCUCCUUACGAGGAGACUAAUCUCUCAUGAAAUAUUUUA | 979 |
| 2 | T149_CasPhi.12 | CTTT | GAUUGCUCCUUACGAGGAGACCUUUAGAAGAAUAUUUCUUUUA | 980 |
| 2 | T150_CasPhi.12 | TTTC | GAUUGCUCCUUACGAGGAGACUUUAGAAGAAUAUUUCAUUUUA | 981 |
| 2 | T151_CasPhi.12 | GTTA | GAUUGCUCCUUACGAGGAGACAUCGAAUAAGUAAUGUGUUUUA | 982 |
| 2 | T152_CasPhi.12 | ATTA | GAUUGCUCCUUACGAGGAGACCUUAUUCGAUUAACACUUUUUA | 983 |
| 2 | T153_CasPhi.12 | CTTA | GAUUGCUCCUUACGAGGAGACUUCGAUUAACACUUUGAUUUUA | 984 |
| 2 | T154_CasPhi.12 | GTTA | GAUUGCUCCUUACGAGGAGACGAAGAUCUGAGCUCUGAUUUUA | 985 |
| 2 | T155_CasPhi.12 | ATTC | GAUUGCUCCUUACGAGGAGACUACCCACUAAAGUUAAUUUUUA | 986 |
| 2 | T156_CasPhi.12 | ATTC | GAUUGCUCCUUACGAGGAGACACCAAAUGGAUUAAGAUUUUUA | 987 |
| 2 | T157_CasPhi.12 | CTTC | GAUUGCUCCUUACGAGGAGACAAAGUGUUAAUCGAAUAUUUUA | 988 |
| 2 | T158_CasPhi.12 | TTTA | GAUUGCUCCUUACGAGGAGACAAAGAAAUUCUACCCACUUUUA | 989 |
| 2 | T159_CasPhi.12 | CTTT | GAUUGCUCCUUACGAGGAGACAGUGGGUAGAAUUUCUUUUUUA | 990 |
| 2 | T160_CasPhi.12 | ATTC | GAUUGCUCCUUACGAGGAGACGAUUAACACUUUGAAGAUUUUA | 991 |
| 2 | T161_CasPhi.12 | ATTC | GAUUGCUCCUUACGAGGAGACAUGAACAUCUUAAUCCAUUUUA | 992 |
| 2 | T162_CasPhi.12 | ATTT | GAUUGCUCCUUACGAGGAGACGGUGAAUAUAUUAUUGGUUUUA | 993 |
| 2 | T163_CasPhi.12 | CTTT | GAUUGCUCCUUACGAGGAGACAACAGAAAAGCAUACACUUUUA | 994 |
| 2 | T164_CasPhi.12 | CTTT | GAUUGCUCCUUACGAGGAGACUCUGUUAAAGAGGAAGUUUUUA | 995 |
| 2 | T165_CasPhi.12 | TTTC | GAUUGCUCCUUACGAGGAGACUGUUAAAGAGGAAGUUAUUUUA | 996 |
| 2 | T166_CasPhi.12 | ATTA | GAUUGCUCCUUACGAGGAGACUCUUCAAAGUGUUAAUCUUUUA | 997 |
| 2 | T167_CasPhi.12 | TTTT | GAUUGCUCCUUACGAGGAGACCUGUUAAAGAGGAAGUUUUUUA | 998 |
| 2 | T168_CasPhi.12 | TTTG | GAUUGCUCCUUACGAGGAGACGUGAAUAUAUUAUUGGAUUUUA | 999 |
| 2 | T169_CasPhi.12 | CTTA | GAUUGCUCCUUACGAGGAGACAUCCAUUUGGUGAAUAUUUUUA | 1000 |
| 2 | T170_CasPhi.12 | TTTA | GAUUGCUCCUUACGAGGAGACGUGGGUAGAAUUUCUUUUUUUA | 1001 |
| 2 | T171_CasPhi.12 | GTTA | GAUUGCUCCUUACGAGGAGACAAGAGGAAGUUAGAAGAUUUUA | 1002 |
| 2 | T172_CasPhi.12 | CTTC | GAUUGCUCCUUACGAGGAGACUAACUUCCUCUUUAACAUUUUA | 1003 |
| 2 | T173_CasPhi.12 | TTTG | GAUUGCUCCUUACGAGGAGACAAGAUAAUUCAUGAACAUUUUA | 1004 |
| 2 | T174_CasPhi.12 | ATTA | GAUUGCUCCUUACGAGGAGACAGAUGUUCAUGAAUUAUUUUUA | 1005 |
| 2 | T175_CasPhi.12 | CTTC | GAUUGCUCCUUACGAGGAGACUAAAUUAACUUUAGUGGUUUUA | 1006 |
| 2 | T176_CasPhi.12 | CTTT | GAUUGCUCCUUACGAGGAGACAAAAGAAAUUCUACCCAUUUUA | 1007 |
| 2 | T177_CasPhi.12 | CTTC | GAUUGCUCCUUACGAGGAGACCUCUUUAACAGAAAAGCUUUUA | 1008 |
| 2 | T178_CasPhi.12 | TTTA | GAUUGCUCCUUACGAGGAGACGAAGUAAAAUAUAAUAGUUUUA | 1009 |
| 2 | T179_CasPhi.12 | TTTA | GAUUGCUCCUUACGAGGAGACACAGAAAAGCAUACACAUUUUA | 1010 |
| 2 | T180_CasPhi.12 | GTTC | GAUUGCUCCUUACGAGGAGACAUGAAUUAUCUUCAAAGUUUUA | 1011 |
| 2 | T181_CasPhi.12 | ATTA | GAUUGCUCCUUACGAGGAGACACACUUUGAAGAUAAUUUUUUA | 1012 |
| 2 | T182_CasPhi.12 | ATTT | GAUUGCUCCUUACGAGGAGACUACUUCUAAAUUAACUUUUUUA | 1013 |
| 2 | T183_CasPhi.12 | TTTA | GAUUGCUCCUUACGAGGAGACCUUCUAAAUUAACUUUAUUUUA | 1014 |
| 2 | T184_CasPhi.12 | TTTT | GAUUGCUCCUUACGAGGAGACACUUCUAAAUUAACUUUUUUUA | 1015 |
| 2 | T185_CasPhi.12 | ATTT | GAUUGCUCCUUACGAGGAGACAGAAGUAAAAUAUAAUAUUUUA | 1016 |
| 2 | T186_CasPhi.12 | GTTA | GAUUGCUCCUUACGAGGAGACAUUUAGAAGUAAAAUAUUUUUA | 1017 |
| 2 | T187_CasPhi.12 | ATTC | GAUUGCUCCUUACGAGGAGACCUUUAAAAGAAAUUCUAUUUUA | 1018 |
| 2 | T188_CasPhi.12 | ATTA | GAUUGCUCCUUACGAGGAGACUUGGAUUUCUAUUAUAUUUUUA | 1019 |
| 2 | T189_CasPhi.12 | ATTT | GAUUGCUCCUUACGAGGAGACCUAUUAUAUUUUACUUCUUUUA | 1020 |
| 2 | T190_CasPhi.12 | ATTG | GAUUGCUCCUUACGAGGAGACGAUUUCUAUUAUAUUUUUUUUA | 1021 |
| 2 | T191_CasPhi.12 | ATTA | GAUUGCUCCUUACGAGGAGACUAUUUUACUUCUAAAUUUUUUA | 1022 |
| 2 | T192_CasPhi.12 | TTTC | GAUUGCUCCUUACGAGGAGACUAUUAUAUUUUACUUCUUUUUA | 1023 |
| 2 | T193_CasPhi.12 | CTTA | GAUUGCUCCUUACGAGGAGACCAGGCUCCAAUAGUGGUUUUUA | 1024 |
| 2 | T194_CasPhi.12 | ATTG | GAUUGCUCCUUACGAGGAGACGAGCCUGUAAGUAUACUUUUUA | 1025 |
| 2 | T195_CasPhi.12 | TTTG | GAUUGCUCCUUACGAGGAGACAACAAAUAGCUAGAGCCUUUUA | 1026 |
| 2 | T196_CasPhi.12 | CTTT | GAUUGCUCCUUACGAGGAGACGUGUUAUUGACUGUGCAUUUUA | 1027 |
| 2 | T197_CasPhi.12 | TTTT | GAUUGCUCCUUACGAGGAGACGAACAAAUAGCUAGAGCUUUUA | 1028 |
| 2 | T198_CasPhi.12 | CTTT | GAUUGCUCCUUACGAGGAGACGGCUCUAGCUAUUUGUUUUUUA | 1029 |
| 2 | T199_CasPhi.12 | TTTT | GAUUGCUCCUUACGAGGAGACGCACAGUCAAUAACACAUUUUA | 1030 |
| 2 | T200_CasPhi.12 | TTTT | GAUUGCUCCUUACGAGGAGACUGCACAGUCAAUAACACUUUUA | 1031 |
| 2 | T201_CasPhi.12 | ATTT | GAUUGCUCCUUACGAGGAGACGUUCAAAAGUGCAACUAUUUUA | 1032 |
| 2 | T202_CasPhi.12 | TTTG | GAUUGCUCCUUACGAGGAGACGCUCUAGCUAUUUGUUCUUUUA | 1033 |
| 2 | T203_CasPhi.12 | CTTT | GAUUGCUCCUUACGAGGAGACUUGCACAGUCAAUAACAUUUUA | 1034 |
| 2 | T204_CasPhi.12 | CTTT | GAUUGCUCCUUACGAGGAGACUGAACAAAUAGCUAGAGUUUUA | 1035 |
| 2 | T205_CasPhi.12 | TTTG | GAUUGCUCCUUACGAGGAGACUGUUAUUGACUGUGCAAUUUUA | 1036 |
| 2 | T206_CasPhi.12 | TTTG | GAUUGCUCCUUACGAGGAGACCACAGUCAAUAACACAAUUUUA | 1037 |
| 2 | T207_CasPhi.12 | TTTC | GAUUGCUCCUUACGAGGAGACUCUCUCACCCAGUCAUCUUUUA | 1038 |
| 2 | T208_CasPhi.12 | GTTA | GAUUGCUCCUUACGAGGAGACUUGACUGUGCAAAAAGUUUUUA | 1039 |
| 2 | T209_CasPhi.12 | ATTC | GAUUGCUCCUUACGAGGAGACUCUUUGGCUCUAGCUAUUUUUA | 1040 |
| 2 | T210_CasPhi.12 | ATTT | GAUUGCUCCUUACGAGGAGACUCUCUCUCACCCAGUCAUUUUA | 1041 |
| 2 | T211_CasPhi.12 | ATTG | GAUUGCUCCUUACGAGGAGACACUGUGCAAAAAGUCUUUUUUA | 1042 |
| 2 | T212_CasPhi.12 | TTTG | GAUUGCUCCUUACGAGGAGACUUCAAAAGUGCAACUAUUUUUA | 1043 |
| 2 | T213_CasPhi.12 | CTTC | GAUUGCUCCUUACGAGGAGACAUAGUUGCACUUUUGAAUUUUA | 1044 |
| 2 | T214_CasPhi.12 | GTTT | GAUUGCUCCUUACGAGGAGACCAAUUCUAAAGAUAGAGUUUUA | 1045 |
| 2 | T215_CasPhi.12 | ATTT | GAUUGCUCCUUACGAGGAGACGUUUCAAUUCUAAAGAUUUUUA | 1046 |
| 2 | T216_CasPhi.12 | GTTG | GAUUGCUCCUUACGAGGAGACCACUUUUGAACAAAUAGUUUUA | 1047 |
| 2 | T217_CasPhi.12 | GTTC | GAUUGCUCCUUACGAGGAGACAAAAGUGCAACUAUGAAUUUUA | 1048 |
| 2 | T218_CasPhi.12 | TTTT | GAUUGCUCCUUACGAGGAGACCUCUCUCACCCAGUCAUUUUUA | 1049 |
| 2 | T219_CasPhi.12 | CTTT | GAUUGCUCCUUACGAGGAGACAGAAUUGAAACAAAUUUUUUUA | 1050 |
| 2 | T220_CasPhi.12 | TTTA | GAUUGCUCCUUACGAGGAGACGAAUUGAAACAAAUUUUUUUUA | 1051 |
| 2 | T221_CasPhi.12 | TTTA | GAUUGCUCCUUACGAGGAGACUCUCUAUCUUUAGAAUUUUUUA | 1052 |
| 2 | T222_CasPhi.12 | TTTC | GAUUGCUCCUUACGAGGAGACAAUUCUAAAGAUAGAGAUUUUA | 1053 |
| 2 | T223_CasPhi.12 | ATTC | GAUUGCUCCUUACGAGGAGACUAAAGAUAGAGAUAAACUUUUA | 1054 |
| 2 | T224_CasPhi.12 | ATTG | GAUUGCUCCUUACGAGGAGACAAACAAAUUUUCUCUCUUUUUA | 1055 |
| 2 | T225_CasPhi.12 | TTTG | GAUUGCUCCUUACGAGGAGACUUUCAAUUCUAAAGAUAUUUUA | 1056 |
| 2 | T226_CasPhi.12 | TTTA | GAUUGCUCCUUACGAGGAGACGCUCCUACUCAGACUGUUUUUA | 1057 |
| 2 | T227_CasPhi.12 | TTTT | GAUUGCUCCUUACGAGGAGACAGCUCCUACUCAGACUGUUUUA | 1058 |
| 2 | T228_CasPhi.12 | GTTT | GAUUGCUCCUUACGAGGAGACGGCUCAAAUUGUUACUCUUUUA | 1059 |
| 2 | T229_CasPhi.12 | ATTT | GAUUGCUCCUUACGAGGAGACUAGCUCCUACUCAGACUUUUUA | 1060 |
| 2 | T230_CasPhi.12 | ATTT | GAUUGCUCCUUACGAGGAGACGAGCCAAACUCUUAUUCUUUUA | 1061 |
| 2 | T231_CasPhi.12 | TTTT | GAUUGCUCCUUACGAGGAGACGGGUUUUUGCAAAAAGGUUUUA | 1062 |
| 2 | T232_CasPhi.12 | TTTG | GAUUGCUCCUUACGAGGAGACGCUCAAAUUGUUACUCUUUUUA | 1063 |
| 2 | T233_CasPhi.12 | ATTG | GAUUGCUCCUUACGAGGAGACAAAUUGGCUCUUUAGCUUUUUA | 1064 |
| 2 | T234_CasPhi.12 | ATTG | GAUUGCUCCUUACGAGGAGACGCUCUUUAGCUUGUGUUUUUUA | 1065 |
| 2 | T235_CasPhi.12 | TTTG | GAUUGCUCCUUACGAGGAGACAGCCAAACUCUUAUUCAUUUUA | 1066 |
| 2 | T236_CasPhi.12 | TTTT | GAUUGCUCCUUACGAGGAGACCCUUUUUGCAAAAACCCUUUUA | 1067 |
| 2 | T237_CasPhi.12 | ATTG | GAUUGCUCCUUACGAGGAGACAAGAGUAACAAUUUGAGUUUUA | 1068 |
| 2 | T238_CasPhi.12 | ATTA | GAUUGCUCCUUACGAGGAGACGAAACACAAGCUAAAGAUUUUA | 1069 |
| 2 | T239_CasPhi.12 | ATTT | GAUUGCUCCUUACGAGGAGACGACUUAUUGUUAUUGAAUUUUA | 1070 |
| 2 | T240_CasPhi.12 | TTTT | GAUUGCUCCUUACGAGGAGACGCAAAAACCCAAAAUAUUUUUA | 1071 |
| 2 | T241_CasPhi.12 | TTTG | GAUUGCUCCUUACGAGGAGACGGUUUUUGCAAAAAGGAUUUUA | 1072 |
| 2 | T242_CasPhi.12 | TTTT | GAUUGCUCCUUACGAGGAGACUCCUUUUUGCAAAAACCUUUUA | 1073 |
| 2 | T243_CasPhi.12 | ATTT | GAUUGCUCCUUACGAGGAGACCAAUAACAAUAAGUCAAUUUUA | 1074 |
| 2 | T244_CasPhi.12 | TTTA | GAUUGCUCCUUACGAGGAGACAUUGAAGAGUAACAAUUUUUUA | 1075 |
| 2 | T245_CasPhi.12 | GTTA | GAUUGCUCCUUACGAGGAGACUUGAAAUUGGCUCUUUAUUUUA | 1076 |
| 2 | T246_CasPhi.12 | TTTT | GAUUGCUCCUUACGAGGAGACUGCAAAAACCCAAAAUAUUUUA | 1077 |
| 2 | T247_CasPhi.12 | ATTG | GAUUGCUCCUUACGAGGAGACUUAUUGAAAUUGGCUCUUUUUA | 1078 |
| 2 | T248_CasPhi.12 | CTTT | GAUUGCUCCUUACGAGGAGACAGCUUGUGUUUCUAAUUUUUUA | 1079 |
| 2 | T249_CasPhi.12 | CTTT | GAUUGCUCCUUACGAGGAGACUUGCAAAAACCCAAAAUUUUUA | 1080 |
| 2 | T250_CasPhi.12 | CTTA | GAUUGCUCCUUACGAGGAGACUUGUUAUUGAAAUUGGCUUUUA | 1081 |
| 2 | T251_CasPhi.12 | TTTA | GAUUGCUCCUUACGAGGAGACGCUUGUGUUUCUAAUUUUUUUA | 1082 |
| 2 | T252_CasPhi.12 | CTTA | GAUUGCUCCUUACGAGGAGACUUCAUGACAUUAUAUAUUUUUA | 1083 |
| 2 | T253_CasPhi.12 | GTTA | GAUUGCUCCUUACGAGGAGACCUCUUCAAUUAAAUUUGUUUUA | 1084 |
| 2 | T254_CasPhi.12 | ATTG | GAUUGCUCCUUACGAGGAGACUUACUCUUCAAUUAAAUUUUUA | 1085 |
| 2 | T255_CasPhi.12 | TTTG | GAUUGCUCCUUACGAGGAGACCAAAAACCCAAAAUAUUUUUUA | 1086 |
| 2 | T256_CasPhi.12 | TTTT | GAUUGCUCCUUACGAGGAGACUUCCUUUUUGCAAAAACUUUUA | 1087 |
| 2 | T257_CasPhi.12 | TTTC | GAUUGCUCCUUACGAGGAGACCUUUUUGCAAAAACCCAUUUUA | 1088 |
| 2 | T258_CasPhi.12 | TTTG | GAUUGCUCCUUACGAGGAGACACUUAUUGUUAUUGAAAUUUUA | 1089 |
| 2 | T259_CasPhi.12 | ATTA | GAUUGCUCCUUACGAGGAGACAAUUUGACUUAUUGUUAUUUUA | 1090 |
| 2 | T260_CasPhi.12 | CTTC | GAUUGCUCCUUACGAGGAGACAAUUAAAUUUGACUUAUUUUUA | 1091 |
| 2 | T261_CasPhi.12 | CTTT | GAUUGCUCCUUACGAGGAGACUUUCCUUUUUGCAAAAAUUUUA | 1092 |
| 2 | T262_CasPhi.12 | TTTT | GAUUGCUCCUUACGAGGAGACGCAAAAAGGAAAAAAGAUUUUA | 1093 |
| 2 | T263_CasPhi.12 | TTTT | GAUUGCUCCUUACGAGGAGACUCUUUUUCUUCUUUUUUUUUUA | 1094 |
| 2 | T264_CasPhi.12 | TTTG | GAUUGCUCCUUACGAGGAGACCAAAAAGGAAAAAAGAAUUUUA | 1095 |
| 2 | T265_CasPhi.12 | CTTC | GAUUGCUCCUUACGAGGAGACUUUUUUCCUUUUUGCAAUUUUA | 1096 |
| 2 | T266_CasPhi.12 | TTTC | GAUUGCUCCUUACGAGGAGACUUCUUUUUUCCUUUUUGUUUUA | 1097 |
| 2 | T267_CasPhi.12 | CTTG | GAUUGCUCCUUACGAGGAGACUGUUUCUAAUUUUUCUUUUUUA | 1098 |
| 2 | T268_CasPhi.12 | GTTT | GAUUGCUCCUUACGAGGAGACCUAAUUUUUCUUUUUCUUUUUA | 1099 |
| 2 | T269_CasPhi.12 | TTTC | GAUUGCUCCUUACGAGGAGACUUUUUCUUCUUUUUUCCUUUUA | 1100 |
| 2 | T270_CasPhi.12 | TTTT | GAUUGCUCCUUACGAGGAGACUCUUCUUUUUUCCUUUUUUUUA | 1101 |
| 2 | T271_CasPhi.12 | TTTC | GAUUGCUCCUUACGAGGAGACUAAUUUUUCUUUUUCUUUUUUA | 1102 |
| 2 | T272_CasPhi.12 | CTTT | GAUUGCUCCUUACGAGGAGACUUCUUCUUUUUUCCUUUUUUUA | 1103 |
| 2 | T273_CasPhi.12 | TTTT | GAUUGCUCCUUACGAGGAGACCUUCUUUUUUCCUUUUUUUUUA | 1104 |
| 2 | T274_CasPhi.12 | TTTT | GAUUGCUCCUUACGAGGAGACCUUUUUCUUCUUUUUUCUUUUA | 1105 |
| 2 | T275_CasPhi.12 | ATTT | GAUUGCUCCUUACGAGGAGACUUCUUUUUCUUCUUUUUUUUUA | 1106 |
| 2 | T276_CasPhi.12 | ATTT | GAUUGCUCCUUACGAGGAGACCCCACCAGUUCUUAGGCUUUUA | 1107 |
| 2 | T277_CasPhi.12 | GTTG | GAUUGCUCCUUACGAGGAGACCCUAAGAACUGGUGGGAUUUUA | 1108 |
| 2 | T278_CasPhi.12 | TTTC | GAUUGCUCCUUACGAGGAGACCCACCAGUUCUUAGGCAUUUUA | 1109 |
| 2 | T279_CasPhi.12 | CTTG | GAUUGCUCCUUACGAGGAGACAUGAUCAUCUCGUUGAUUUUUA | 1110 |
| 2 | T280_CasPhi.12 | CTTC | GAUUGCUCCUUACGAGGAGACUGCUUGAUGAUCAUCUCUUUUA | 1111 |
| 2 | T281_CasPhi.12 | TTTC | GAUUGCUCCUUACGAGGAGACCACCAAUCACUUUACUCUUUUA | 1112 |
| 2 | T282_CasPhi.12 | ATTT | GAUUGCUCCUUACGAGGAGACUCCACCAAUCACUUUACUUUUA | 1113 |
| 2 | T283_CasPhi.12 | CTTT | GAUUGCUCCUUACGAGGAGACACUCUCCUAGACCAUUUUUUUA | 1114 |
| 2 | T284_CasPhi.12 | TTTT | GAUUGCUCCUUACGAGGAGACCCACCAAUCACUUUACUUUUUA | 1115 |
| 2 | T285_CasPhi.12 | TTTA | GAUUGCUCCUUACGAGGAGACCUCUCCUAGACCAUUUCUUUUA | 1116 |
| 2 | T286_CasPhi.12 | GTTT | GAUUGCUCCUUACGAGGAGACGCUGAGAGAGAAACAGUUUUUA | 1117 |
| 2 | T287_CasPhi.12 | ATTA | GAUUGCUCCUUACGAGGAGACCUCUCACUAUUCAGCCUUUUUA | 1118 |
| 2 | T288_CasPhi.12 | TTTC | GAUUGCUCCUUACGAGGAGACUCAUACCUUCUGCUUGAUUUUA | 1119 |
| 2 | T289_CasPhi.12 | GTTC | GAUUGCUCCUUACGAGGAGACUUAGGCAACUGUUUCUCUUUUA | 1120 |
| 2 | T290_CasPhi.12 | TTTT | GAUUGCUCCUUACGAGGAGACCUCAUACCUUCUGCUUGUUUUA | 1121 |
| 2 | T291_CasPhi.12 | CTTA | GAUUGCUCCUUACGAGGAGACGGCAACUGUUUCUCUCUUUUUA | 1122 |
| 2 | T292_CasPhi.12 | GTTT | GAUUGCUCCUUACGAGGAGACCUCUCUCAGCAAACACAUUUUA | 1123 |
| 2 | T293_CasPhi.12 | TTTT | GAUUGCUCCUUACGAGGAGACAAAGAAAAACUUCUGCCUUUUA | 1124 |
| 2 | T294_CasPhi.12 | TTTC | GAUUGCUCCUUACGAGGAGACUCUCUCAGCAAACACAUUUUUA | 1125 |
| 2 | T295_CasPhi.12 | TTTA | GAUUGCUCCUUACGAGGAGACAAGAAAAACUUCUGCCAUUUUA | 1126 |
| 2 | T296_CasPhi.12 | TTTT | GAUUGCUCCUUACGAGGAGACUUCUCAUACCUUCUGCUUUUUA | 1127 |
| 2 | T297_CasPhi.12 | TTTG | GAUUGCUCCUUACGAGGAGACCUGAGAGAGAAACAGUUUUUUA | 1128 |
| 2 | T298_CasPhi.12 | GTTG | GAUUGCUCCUUACGAGGAGACGCAGAAGUUUUUCUUUAUUUUA | 1129 |
| 2 | T299_CasPhi.12 | ATTT | GAUUGCUCCUUACGAGGAGACUAAAGAAAAACUUCUGCUUUUA | 1130 |
| 2 | T300_CasPhi.12 | ATTG | GAUUGCUCCUUACGAGGAGACGUGGAAAAUCUUCAUUUUUUUA | 1131 |
| 2 | T301_CasPhi.12 | ATTT | GAUUGCUCCUUACGAGGAGACUUUCUCAUACCUUCUGCUUUUA | 1132 |
| 2 | T302_CasPhi.12 | TTTA | GAUUGCUCCUUACGAGGAGACAAAUGAAGAUUUUCCACUUUUA | 1133 |
| 2 | T303_CasPhi.12 | CTTC | GAUUGCUCCUUACGAGGAGACUGCCAACUUUUAUCAUUUUUUA | 1134 |
| 2 | T304_CasPhi.12 | TTTT | GAUUGCUCCUUACGAGGAGACUCUCAUACCUUCUGCUUUUUUA | 1135 |
| 2 | T305_CasPhi.12 | CTTT | GAUUGCUCCUUACGAGGAGACAAAAUGAAGAUUUUCCAUUUUA | 1136 |
| 2 | T306_CasPhi.12 | TTTA | GAUUGCUCCUUACGAGGAGACUCAUUUUUUCUCAUACCUUUUA | 1137 |
| 2 | T307_CasPhi.12 | CTTT | GAUUGCUCCUUACGAGGAGACUAUCAUUUUUUCUCAUAUUUUA | 1138 |
| 2 | T308_CasPhi.12 | TTTT | GAUUGCUCCUUACGAGGAGACAUCAUUUUUUCUCAUACUUUUA | 1139 |
| 2 | T309_CasPhi.12 | TTTT | GAUUGCUCCUUACGAGGAGACCUUUAAAAUGAAGAUUUUUUUA | 1140 |
| 2 | T310_CasPhi.12 | TTTT | GAUUGCUCCUUACGAGGAGACUCUUUAAAAUGAAGAUUUUUUA | 1141 |
| 2 | T311_CasPhi.12 | GTTT | GAUUGCUCCUUACGAGGAGACUUCUUUAAAAUGAAGAUUUUUA | 1142 |
| 2 | T312_CasPhi.12 | CTTC | GAUUGCUCCUUACGAGGAGACAUUUUAAAGAAAAACUUUUUUA | 1143 |
| 2 | T313_CasPhi.12 | TTTC | GAUUGCUCCUUACGAGGAGACUUUAAAAUGAAGAUUUUUUUUA | 1144 |
| 2 | T314_CasPhi.12 | GTTC | GAUUGCUCCUUACGAGGAGACAUCAUCCUAGCCAUAACUUUUA | 1145 |
| 2 | T315_CasPhi.12 | ATTG | GAUUGCUCCUUACGAGGAGACUGUUAUGGCUAGGAUGAUUUUA | 1146 |
| 2 | T316_CasPhi.12 | TTTA | GAUUGCUCCUUACGAGGAGACAAUGUAACUUCCAAACGUUUUA | 1147 |
| 2 | T317_CasPhi.12 | ATTC | GAUUGCUCCUUACGAGGAGACAUUGUGUUAUGGCUAGGUUUUA | 1148 |
| 2 | T318_CasPhi.12 | GTTG | GAUUGCUCCUUACGAGGAGACUUCAUCAUCCUAGCCAUUUUUA | 1149 |
| 2 | T319_CasPhi.12 | CTTC | GAUUGCUCCUUACGAGGAGACAUCCCACUGAUUCUGAAUUUUA | 1150 |
| 2 | T320_CasPhi.12 | TTTA | GAUUGCUCCUUACGAGGAGACAAAUGUCUCCUCCAGACUUUUA | 1151 |
| 2 | T321_CasPhi.12 | ATTA | GAUUGCUCCUUACGAGGAGACUUCAUUGUGUUAUGGCUUUUUA | 1152 |
| 2 | T322_CasPhi.12 | ATTC | GAUUGCUCCUUACGAGGAGACAGAAUCAGUGGGAUGAAUUUUA | 1153 |
| 2 | T323_CasPhi.12 | ATTT | GAUUGCUCCUUACGAGGAGACAAAAUGUCUCCUCCAGAUUUUA | 1154 |
| 2 | T324_CasPhi.12 | CTTC | GAUUGCUCCUUACGAGGAGACCAAACGUUAUCUCACAUUUUUA | 1155 |
| 2 | T325_CasPhi.12 | GTTT | GAUUGCUCCUUACGAGGAGACGGAAGUUACAUUUAAAAUUUUA | 1156 |
| 2 | T326_CasPhi.12 | ATTC | GAUUGCUCCUUACGAGGAGACUGAAUUCUUUCAACUAGUUUUA | 1157 |
| 2 | T327_CasPhi.12 | CTTT | GAUUGCUCCUUACGAGGAGACCAACUAGAAUAAAAGGAUUUUA | 1158 |
| 2 | T328_CasPhi.12 | GTTA | GAUUGCUCCUUACGAGGAGACUCUCACAUUUAUGUUGCUUUUA | 1159 |
| 2 | T329_CasPhi.12 | TTTT | GAUUGCUCCUUACGAGGAGACAUUCUAGUUGAAAGAAUUUUUA | 1160 |
| 2 | T330_CasPhi.12 | GTTA | GAUUGCUCCUUACGAGGAGACCAUUUAAAAUGUCUCCUUUUUA | 1161 |
| 2 | T331_CasPhi.12 | ATTC | GAUUGCUCCUUACGAGGAGACUAGUUGAAAGAAUUCAGUUUUA | 1162 |
| 2 | T332_CasPhi.12 | GTTG | GAUUGCUCCUUACGAGGAGACAAAGAAUUCAGAAUCAGUUUUA | 1163 |
| 2 | T333_CasPhi.12 | TTTA | GAUUGCUCCUUACGAGGAGACAAUAAGCAACAUAAAUGUUUUA | 1164 |
| 2 | T334_CasPhi.12 | TTTC | GAUUGCUCCUUACGAGGAGACAACUAGAAUAAAAGGAAUUUUA | 1165 |
| 2 | T335_CasPhi.12 | TTTT | GAUUGCUCCUUACGAGGAGACCCUUUUAUUCUAGUUGAUUUUA | 1166 |
| 2 | T336_CasPhi.12 | TTTA | GAUUGCUCCUUACGAGGAGACUUCUAGUUGAAAGAAUUUUUUA | 1167 |
| 2 | T337_CasPhi.12 | CTTT | GAUUGCUCCUUACGAGGAGACUAUUCUAGUUGAAAGAAUUUUA | 1168 |
| 2 | T338_CasPhi.12 | TTTT | GAUUGCUCCUUACGAGGAGACUUAAAUAAGCAACAUAAUUUUA | 1169 |
| 2 | T339_CasPhi.12 | TTTT | GAUUGCUCCUUACGAGGAGACUAAAUAAGCAACAUAAAUUUUA | 1170 |
| 2 | T340_CasPhi.12 | ATTT | GAUUGCUCCUUACGAGGAGACUUUAAAUAAGCAACAUAUUUUA | 1171 |
| 2 | T341_CasPhi.12 | TTTA | GAUUGCUCCUUACGAGGAGACAAAAAUUAUUCAUUGUGUUUUA | 1172 |
| 2 | T342_CasPhi.12 | TTTT | GAUUGCUCCUUACGAGGAGACAAAUAAGCAACAUAAAUUUUUA | 1173 |
| 2 | T343_CasPhi.12 | TTTT | GAUUGCUCCUUACGAGGAGACUCCUUUUAUUCUAGUUGUUUUA | 1174 |
| 2 | T344_CasPhi.12 | TTTG | GAUUGCUCCUUACGAGGAGACGAAGUUACAUUUAAAAUUUUUA | 1175 |
| 2 | T345_CasPhi.12 | TTTC | GAUUGCUCCUUACGAGGAGACCUUUUAUUCUAGUUGAAUUUUA | 1176 |
| 2 | T346_CasPhi.12 | ATTT | GAUUGCUCCUUACGAGGAGACACUACUAUAUAUUUAUUUUUUA | 1177 |
| 2 | T347_CasPhi.12 | ATTT | GAUUGCUCCUUACGAGGAGACAAAAAAUUAUUCAUUGUUUUUA | 1178 |
| 2 | T348_CasPhi.12 | ATTT | GAUUGCUCCUUACGAGGAGACUUCCUUUUAUUCUAGUUUUUUA | 1179 |
| 2 | T349_CasPhi.12 | TTTA | GAUUGCUCCUUACGAGGAGACCUACUAUAUAUUUAUUUUUUUA | 1180 |
| 2 | T350_CasPhi.12 | TTTA | GAUUGCUCCUUACGAGGAGACUUUUUCCUUUUAUUCUAUUUUA | 1181 |
| 2 | T351_CasPhi.12 | ATTT | GAUUGCUCCUUACGAGGAGACAUUUUUCCUUUUAUUCUUUUUA | 1182 |
| 2 | T352_CasPhi.12 | ATTT | GAUUGCUCCUUACGAGGAGACCAGCUUUAACGUGAUUUUUUUA | 1183 |
| 2 | T353_CasPhi.12 | CTTT | GAUUGCUCCUUACGAGGAGACGAUACUAACCUUGGUUUUUUUA | 1184 |
| 2 | T354_CasPhi.12 | GTTC | GAUUGCUCCUUACGAGGAGACAUUUCAGCUUUAACGUGUUUUA | 1185 |
| 2 | T355_CasPhi.12 | TTTG | GAUUGCUCCUUACGAGGAGACAUACUAACCUUGGUUUCUUUUA | 1186 |
| 2 | T356_CasPhi.12 | GTTA | GAUUGCUCCUUACGAGGAGACGUAUCAAAGAUACCUUUUUUUA | 1187 |
| 2 | T357_CasPhi.12 | ATTA | GAUUGCUCCUUACGAGGAGACACAGAAAAUCACGUUAAUUUUA | 1188 |
| 2 | T358_CasPhi.12 | GTTA | GAUUGCUCCUUACGAGGAGACUUAACAGAAAAUCACGUUUUUA | 1189 |
| 2 | T359_CasPhi.12 | ATTT | GAUUGCUCCUUACGAGGAGACUAUCAAAUGUAACCAGUUUUUA | 1190 |
| 2 | T360_CasPhi.12 | ATTA | GAUUGCUCCUUACGAGGAGACAAUUACAAUCUAUGGUAUUUUA | 1191 |
| 2 | T361_CasPhi.12 | TTTT | GAUUGCUCCUUACGAGGAGACAUCAAAUGUAACCAGUAUUUUA | 1192 |
| 2 | T362_CasPhi.12 | TTTC | GAUUGCUCCUUACGAGGAGACAGCUUUAACGUGAUUUUUUUUA | 1193 |
| 2 | T363_CasPhi.12 | TTTA | GAUUGCUCCUUACGAGGAGACACGUGAUUUUCUGUUAAUUUUA | 1194 |
| 2 | T364_CasPhi.12 | CTTT | GAUUGCUCCUUACGAGGAGACAACGUGAUUUUCUGUUAUUUUA | 1195 |
| 2 | T365_CasPhi.12 | TTTA | GAUUGCUCCUUACGAGGAGACUCAAAUGUAACCAGUAUUUUUA | 1196 |
| 2 | T366_CasPhi.12 | ATTA | GAUUGCUCCUUACGAGGAGACUACCAUAGAUUGUAAUUUUUUA | 1197 |
| 2 | T367_CasPhi.12 | ATTA | GAUUGCUCCUUACGAGGAGACCAAUCUAUGGUAUAAUUUUUUA | 1198 |
| 2 | T368_CasPhi.12 | GTTA | GAUUGCUCCUUACGAGGAGACAAGCUGAAAUGAACAGUUUUUA | 1199 |
| 2 | T369_CasPhi.12 | ATTT | GAUUGCUCCUUACGAGGAGACGAUAAAAUUAUACCAUAUUUUA | 1200 |
| 2 | T370_CasPhi.12 | TTTT | GAUUGCUCCUUACGAGGAGACCUGUUAAUAACUUUACAUUUUA | 1201 |
| 2 | T371_CasPhi.12 | ATTT | GAUUGCUCCUUACGAGGAGACUCUGUUAAUAACUUUACUUUUA | 1202 |
| 2 | T372_CasPhi.12 | TTTA | GAUUGCUCCUUACGAGGAGACAAAUAAAAUACUGGUUAUUUUA | 1203 |
| 2 | T373_CasPhi.12 | ATTT | GAUUGCUCCUUACGAGGAGACAAUGAUGUUUAAUGUAAUUUUA | 1204 |
| 2 | T374_CasPhi.12 | TTTA | GAUUGCUCCUUACGAGGAGACAAAAGGUAUCUUUGAUAUUUUA | 1205 |
| 2 | T375_CasPhi.12 | CTTT | GAUUGCUCCUUACGAGGAGACACAUUAAACAUCAUUAAUUUUA | 1206 |
| 2 | T376_CasPhi.12 | TTTA | GAUUGCUCCUUACGAGGAGACAUGUAAAGUUAUUAACAUUUUA | 1207 |
| 2 | T377_CasPhi.12 | TTTG | GAUUGCUCCUUACGAGGAGACAUAAAAUUAUACCAUAGUUUUA | 1208 |
| 2 | T378_CasPhi.12 | ATTT | GAUUGCUCCUUACGAGGAGACUAAAAAGGUAUCUUUGAUUUUA | 1209 |
| 2 | T379_CasPhi.12 | TTTT | GAUUGCUCCUUACGAGGAGACAAAAAGGUAUCUUUGAUUUUUA | 1210 |
| 2 | T380_CasPhi.12 | GTTT | GAUUGCUCCUUACGAGGAGACAAUGUAAAGUUAUUAACUUUUA | 1211 |
| 2 | T381_CasPhi.12 | ATTA | GAUUGCUCCUUACGAGGAGACAACAUCAUUAAAUUACAUUUUA | 1212 |
| 2 | T382_CasPhi.12 | TTTA | GAUUGCUCCUUACGAGGAGACAUGAUGUUUAAUGUAAAUUUUA | 1213 |
| 2 | T383_CasPhi.12 | TTTT | GAUUGCUCCUUACGAGGAGACAAAAUAAAAUACUGGUUUUUUA | 1214 |
| 2 | T384_CasPhi.12 | CTTT | GAUUGCUCCUUACGAGGAGACUUAAAAUAAAAUACUGGUUUUA | 1215 |
| 2 | T385_CasPhi.12 | TTTT | GAUUGCUCCUUACGAGGAGACUAAAAUAAAAUACUGGUUUUUA | 1216 |
| 2 | T386_CasPhi.12 | GTTT | GAUUGCUCCUUACGAGGAGACCUGUGAUUUUCUUUUGGUUUUA | 1217 |
| 2 | T387_CasPhi.12 | TTTC | GAUUGCUCCUUACGAGGAGACUGUUAAUAACUUUACAUUUUUA | 1218 |
| 2 | T388_CasPhi.12 | ATTT | GAUUGCUCCUUACGAGGAGACUAUUUUAAAAAGGUAUCUUUUA | 1219 |
| 2 | T389_CasPhi.12 | ATTG | GAUUGCUCCUUACGAGGAGACUAAUUUAAUGAUGUUUAUUUUA | 1220 |
| 2 | T390_CasPhi.12 | CTTG | GAUUGCUCCUUACGAGGAGACGUUUCUGUGAUUUUCUUUUUUA | 1221 |
| 2 | T391_CasPhi.12 | TTTT | GAUUGCUCCUUACGAGGAGACAUUUUAAAAAGGUAUCUUUUUA | 1222 |
| 2 | T392_CasPhi.12 | TTTA | GAUUGCUCCUUACGAGGAGACUUUUAAAAAGGUAUCUUUUUUA | 1223 |
| 2 | T393_CasPhi.12 | GTTA | GAUUGCUCCUUACGAGGAGACCAUUUGAUAAAAUUAUAUUUUA | 1224 |
| 2 | T394_CasPhi.12 | TTTC | GAUUGCUCCUUACGAGGAGACUGUGAUUUUCUUUUGGAUUUUA | 1225 |
| 2 | T395_CasPhi.12 | NTTN | AUUGCUCCUUACGAGGAGACCAUCUACUGUAUAGGGA | 1226 |
| 2 | T396_CasPhi.12 | NTTN | AUUGCUCCUUACGAGGAGACGCUCUGGCCUGUCCUAA | 1227 |
| 2 | T397_CasPhi.12 | NTTN | AUUGCUCCUUACGAGGAGACCAUUCAACUGUUGCCUC | 1228 |
| 2 | T398_CasPhi.12 | NTTN | AUUGCUCCUUACGAGGAGACUGAAGGUGUUCUUGUAC | 1229 |
| 2 | T399_CasPhi.12 | NTTN | AUUGCUCCUUACGAGGAGACCUUUUGGAUUGCAUCUA | 1230 |
| 2 | T400_CasPhi.12 | NTTN | AUUGCUCCUUACGAGGAGACCUUAGCUUCCAGCCAUU | 1231 |
| 2 | T401_CasPhi.12 | NTTN | AUUGCUCCUUACGAGGAGACUUUUGGAUUGCAUCUAC | 1232 |
| 2 | T402_CasPhi.12 | NTTN | AUUGCUCCUUACGAGGAGACGGAUUGCAUCUACUGUA | 1233 |
| 2 | T403_CasPhi.12 | NTTN | AUUGCUCCUUACGAGGAGACAACAUUUCAUUCAACUG | 1234 |
| 2 | T404_CasPhi.12 | NTTN | AUUGCUCCUUACGAGGAGACUUGUACUUCAUCCCACU | 1235 |
| 2 | T405_CasPhi.12 | NTTN | AUUGCUCCUUACGAGGAGACUCUUUUGGAUUGCAUCU | 1236 |
| 2 | T406_CasPhi.12 | NTTN | AUUGCUCCUUACGAGGAGACACAUUUCAUUCAACUGU | 1237 |
| 2 | T407_CasPhi.12 | NTTN | AUUGCUCCUUACGAGGAGACUUCCUUAGCUUCCAGCC | 1238 |
| 2 | T408_CasPhi.12 | NTTN | AUUGCUCCUUACGAGGAGACGAUUGCAUCUACUGUAU | 1239 |
| 2 | T409_CasPhi.12 | NTTN | AUUGCUCCUUACGAGGAGACCAUGACUCAAGCUUGGC | 1240 |
| 2 | T410_CasPhi.12 | NTTN | AUUGCUCCUUACGAGGAGACAUUCAACUGUUGCCUCC | 1241 |
| 2 | T411_CasPhi.12 | NTTN | AUUGCUCCUUACGAGGAGACUGUUGAAUCCUUUAACA | 1242 |
| 2 | T412_CasPhi.12 | NTTN | AUUGCUCCUUACGAGGAGACUGGAUUGCAUCUACUGU | 1243 |
| 2 | T413_CasPhi.12 | NTTN | AUUGCUCCUUACGAGGAGACGCUUCCAGCCAUUGUGU | 1244 |
| 2 | T414_CasPhi.12 | NTTN | AUUGCUCCUUACGAGGAGACCAGCCAUUGUGUUGAAU | 1245 |
| 2 | T415_CasPhi.12 | NTTN | AUUGCUCCUUACGAGGAGACCCUCCGGUUCUGAAGGU | 1246 |
| 2 | T416_CasPhi.12 | NTTN | AUUGCUCCUUACGAGGAGACUACUUCAUCCCACUGAU | 1247 |
| 2 | T417_CasPhi.12 | NTTN | AUUGCUCCUUACGAGGAGACAACUGUUGCCUCCGGUU | 1248 |
| 2 | T418_CasPhi.12 | NTTN | AUUGCUCCUUACGAGGAGACAAGGAUUCAACACAAUG | 1249 |
| 2 | T419_CasPhi.12 | NTTN | AUUGCUCCUUACGAGGAGACAACACAAUGGCUGGAAG | 1250 |
| 2 | T420_CasPhi.12 | NTTN | AUUGCUCCUUACGAGGAGACGGACAGGCCAGAGCCAA | 1251 |
| 2 | T421_CasPhi.12 | NTTN | AUUGCUCCUUACGAGGAGACAGUCAUGGAAGGAGGGU | 1252 |
| 2 | T422_CasPhi.12 | NTTN | AUUGCUCCUUACGAGGAGACAGAACCGGAGGCAACAG | 1253 |
| 2 | T423_CasPhi.12 | NTTN | AUUGCUCCUUACGAGGAGACUAUAACUUGAUCAAGCA | 1254 |
| 2 | T424_CasPhi.12 | NTTN | AUUGCUCCUUACGAGGAGACAUCAAGCAGAGAAAGCC | 1255 |
| 2 | T425_CasPhi.12 | NTTN | AUUGCUCCUUACGAGGAGACUAGUUUGGAGAUGGCAG | 1256 |
| 2 | T426_CasPhi.12 | NTTN | AUUGCUCCUUACGAGGAGACGAGAUGGCAGUUUCCUU | 1257 |
| 2 | T427_CasPhi.12 | NTTN | AUUGCUCCUUACGAGGAGACCCUUAGUAACCACAGGU | 1258 |
| 2 | T428_CasPhi.12 | NTTN | AUUGCUCCUUACGAGGAGACAUAUCCUCAAGGUCACC | 1259 |
| 2 | T429_CasPhi.12 | NTTN | AUUGCUCCUUACGAGGAGACCUUAGUAACCACAGGUU | 1260 |
| 2 | T430_CasPhi.12 | NTTN | AUUGCUCCUUACGAGGAGACUGUCCAAGCCCGGUUGA | 1261 |
| 2 | T431_CasPhi.12 | NTTN | AUUGCUCCUUACGAGGAGACCUAGUUUGGAGAUGGCA | 1262 |
| 2 | T432_CasPhi.12 | NTTN | AUUGCUCCUUACGAGGAGACAAAUCUGCCAGAGCAGG | 1263 |
| 2 | T433_CasPhi.12 | NTTN | AUUGCUCCUUACGAGGAGACUAACUUGAUCAAGCAGA | 1264 |
| 2 | T434_CasPhi.12 | NTTN | AUUGCUCCUUACGAGGAGACGUAACCACAGGUUGUGU | 1265 |
| 2 | T435_CasPhi.12 | NTTN | AUUGCUCCUUACGAGGAGACUGUCACCAGAGUAACAG | 1266 |
| 2 | T436_CasPhi.12 | NTTN | AUUGCUCCUUACGAGGAGACAUAACUUGAUCAAGCAG | 1267 |
| 2 | T437_CasPhi.12 | NTTN | AUUGCUCCUUACGAGGAGACGGAGAUGGCAGUUUCCU | 1268 |
| 2 | T438_CasPhi.12 | NTTN | AUUGCUCCUUACGAGGAGACCUCUGGUGACACAACCU | 1269 |
| 2 | T439_CasPhi.12 | NTTN | AUUGCUCCUUACGAGGAGACAUGUUGGAGGUACCUGC | 1270 |
| 2 | T440_CasPhi.12 | NTTN | AUUGCUCCUUACGAGGAGACCUCUGCUUGAUCAAGUU | 1271 |
| 2 | T441_CasPhi.12 | NTTN | AUUGCUCCUUACGAGGAGACUCUGCUUGAUCAAGUUA | 1272 |
| 2 | T442_CasPhi.12 | NTTN | AUUGCUCCUUACGAGGAGACCUUGAUGUUGGAGGUAC | 1273 |
| 2 | T443_CasPhi.12 | NTTN | AUUGCUCCUUACGAGGAGACAACCGGGCUUGGACAGA | 1274 |
| 2 | T444_CasPhi.12 | NTTN | AUUGCUCCUUACGAGGAGACAGGAUAUCAACGAGAUG | 1275 |
| 2 | T445_CasPhi.12 | NTTN | AUUGCUCCUUACGAGGAGACGAGGUACCUGCUCUGGC | 1276 |
| 2 | T446_CasPhi.12 | NTTN | AUUGCUCCUUACGAGGAGACCAACCGGGCUUGGACAG | 1277 |
| 2 | T447_CasPhi.12 | NTTN | AUUGCUCCUUACGAGGAGACCUAAGGAAACUGCCAUC | 1278 |
| 2 | T448_CasPhi.12 | NTTN | AUUGCUCCUUACGAGGAGACUAAAAUCACAGAGGGUG | 1279 |
| 2 | T449_CasPhi.12 | NTTN | AUUGCUCCUUACGAGGAGACGACAGAACUUACCGACU | 1280 |
| 2 | T450_CasPhi.12 | NTTN | AUUGCUCCUUACGAGGAGACCCGACUGGCUUUCUCUG | 1281 |
| 2 | T451_CasPhi.12 | NTTN | AUUGCUCCUUACGAGGAGACCCGCCUUCCACUCAGAG | 1282 |
| 2 | T452_CasPhi.12 | NTTN | AUUGCUCCUUACGAGGAGACCCCUCAGCUCUUGAAGU | 1283 |
| 2 | T453_CasPhi.12 | NTTN | AUUGCUCCUUACGAGGAGACAAGUAAACGGUUUACCG | 1284 |
| 2 | T454_CasPhi.12 | NTTN | AUUGCUCCUUACGAGGAGACACCGCCUUCCACUCAGA | 1285 |
| 2 | T455_CasPhi.12 | NTTN | AUUGCUCCUUACGAGGAGACGCCCUCAGCUCUUGAAG | 1286 |
| 2 | T456_CasPhi.12 | NTTN | AUUGCUCCUUACGAGGAGACCACUCAGAGCUCAGAUC | 1287 |
| 2 | T457_CasPhi.12 | NTTN | AUUGCUCCUUACGAGGAGACCUUCAAGAGCUGAGGGC | 1288 |
| 2 | T458_CasPhi.12 | NTTN | AUUGCUCCUUACGAGGAGACACUUCAAGAGCUGAGGG | 1289 |
| 2 | T459_CasPhi.12 | NTTN | AUUGCUCCUUACGAGGAGACAAGAGCUGAGGGCAAAG | 1290 |
| 2 | T460_CasPhi.12 | NTTN | AUUGCUCCUUACGAGGAGACUCCUGUAGAAUACUGGC | 1291 |
| 2 | T461_CasPhi.12 | NTTN | AUUGCUCCUUACGAGGAGACGAGGAUUGCUGAAUUAU | 1292 |
| 2 | T462_CasPhi.12 | NTTN | AUUGCUCCUUACGAGGAGACGCCGCUGCCCAAUGCCA | 1293 |
| 2 | T463_CasPhi.12 | NTTN | AUUGCUCCUUACGAGGAGACUUCCCCAGUUGCAUUCA | 1294 |
| 2 | T464_CasPhi.12 | NTTN | AUUGCUCCUUACGAGGAGACUGAGGAUUGCUGAAUUA | 1295 |
| 2 | T465_CasPhi.12 | NTTN | AUUGCUCCUUACGAGGAGACCCGCUGCCCAAUGCCAU | 1296 |
| 2 | T466_CasPhi.12 | NTTN | AUUGCUCCUUACGAGGAGACAGGAUUGCUGAAUUAUU | 1297 |
| 2 | T467_CasPhi.12 | NTTN | AUUGCUCCUUACGAGGAGACCCAAUUUUUCCUGUAGA | 1298 |
| 2 | T468_CasPhi.12 | NTTN | AUUGCUCCUUACGAGGAGACUUCCUGUAGAAUACUGG | 1299 |
| 2 | T469_CasPhi.12 | NTTN | AUUGCUCCUUACGAGGAGACGCAGACCUCCUGCCACC | 1300 |
| 2 | T470_CasPhi.12 | NTTN | AUUGCUCCUUACGAGGAGACCAUUCAAUGUUCUGACA | 1301 |
| 2 | T471_CasPhi.12 | NTTN | AUUGCUCCUUACGAGGAGACUGACAACAGUUUGCCGC | 1302 |
| 2 | T472_CasPhi.12 | NTTN | AUUGCUCCUUACGAGGAGACCUUCCCCAGUUGCAUUC | 1303 |
| 2 | T473_CasPhi.12 | NTTN | AUUGCUCCUUACGAGGAGACCCUGUAGAAUACUGGCA | 1304 |
| 2 | T474_CasPhi.12 | NTTN | AUUGCUCCUUACGAGGAGACCUGAAUUAUUUCUUCCC | 1305 |
| 2 | T475_CasPhi.12 | NTTN | AUUGCUCCUUACGAGGAGACCUGUAGAAUACUGGCAU | 1306 |
| 2 | T476_CasPhi.12 | NTTN | AUUGCUCCUUACGAGGAGACUUGAGGAUUGCUGAAUU | 1307 |
| 2 | T477_CasPhi.12 | NTTN | AUUGCUCCUUACGAGGAGACAAUGUUCUGACAACAGU | 1308 |
| 2 | T478_CasPhi.12 | NTTN | AUUGCUCCUUACGAGGAGACCCCAGUUGCAUUCAAUG | 1309 |
| 2 | T479_CasPhi.12 | NTTN | AUUGCUCCUUACGAGGAGACCAGACCUCCUGCCACCG | 1310 |
| 2 | T480_CasPhi.12 | NTTN | AUUGCUCCUUACGAGGAGACUUUCUUCCCCAGUUGCA | 1311 |
| 2 | T481_CasPhi.12 | NTTN | AUUGCUCCUUACGAGGAGACAGGCUUCCCAAUUUUUC | 1312 |
| 2 | T482_CasPhi.12 | NTTN | AUUGCUCCUUACGAGGAGACGGAAGCCUGAAUCUGCG | 1313 |
| 2 | T483_CasPhi.12 | NTTN | AUUGCUCCUUACGAGGAGACAGCAAUCCUCAAAAACA | 1314 |
| 2 | T484_CasPhi.12 | NTTN | AUUGCUCCUUACGAGGAGACUCAGAACAUUGAAUGCA | 1315 |
| 2 | T485_CasPhi.12 | NTTN | AUUGCUCCUUACGAGGAGACAAUGCAACUGGGGAAGA | 1316 |
| 2 | T486_CasPhi.12 | NTTN | AUUGCUCCUUACGAGGAGACGGCAGCGGCAAACUGUU | 1317 |
| 2 | T487_CasPhi.12 | NTTN | AUUGCUCCUUACGAGGAGACUACAGGAAAAAUUGGGA | 1318 |
| 2 | T488_CasPhi.12 | NTTN | AUUGCUCCUUACGAGGAGACGCCACUGAUUAAAUAUC | 1319 |
| 2 | T489_CasPhi.12 | NTTN | AUUGCUCCUUACGAGGAGACGUGUCUUUCUGAGAAAC | 1320 |
| 2 | T490_CasPhi.12 | NTTN | AUUGCUCCUUACGAGGAGACUCAACAGAUCUGUCAAA | 1321 |
| 2 | T491_CasPhi.12 | NTTN | AUUGCUCCUUACGAGGAGACGUAUUUAGCAUGUUCCC | 1322 |
| 2 | T492_CasPhi.12 | NTTN | AUUGCUCCUUACGAGGAGACUAUUUAGCAUGUUCCCA | 1323 |
| 2 | T493_CasPhi.12 | NTTN | AUUGCUCCUUACGAGGAGACUGUUAGCCACUGAUUAA | 1324 |
| 2 | T494_CasPhi.12 | NTTN | AUUGCUCCUUACGAGGAGACUGAGAAACUGUUCAGCU | 1325 |
| 2 | T495_CasPhi.12 | NTTN | AUUGCUCCUUACGAGGAGACAGCAUGUUCCCAAUUCU | 1326 |
| 2 | T496_CasPhi.12 | NTTN | AUUGCUCCUUACGAGGAGACUAUCAUAAUGAAAACGC | 1327 |
| 2 | T497_CasPhi.12 | NTTN | AUUGCUCCUUACGAGGAGACCUGAGAAACUGUUCAGC | 1328 |
| 2 | T498_CasPhi.12 | NTTN | AUUGCUCCUUACGAGGAGACCCAAUUCUCAGGAAUUU | 1329 |
| 2 | T499_CasPhi.12 | NTTN | AUUGCUCCUUACGAGGAGACUGUCUUUCUGAGAAACU | 1330 |
| 2 | T500_CasPhi.12 | NTTN | AUUGCUCCUUACGAGGAGACUCAGGAAUUUGUGUCUU | 1331 |
| 2 | T501_CasPhi.12 | NTTN | AUUGCUCCUUACGAGGAGACGCAUGUUCCCAAUUCUC | 1332 |
| 2 | T502_CasPhi.12 | NTTN | AUUGCUCCUUACGAGGAGACAGCUUCUGUUAGCCACU | 1333 |
| 2 | T503_CasPhi.12 | NTTN | AUUGCUCCUUACGAGGAGACUCAGAAAGACACAAAUU | 1334 |
| 2 | T504_CasPhi.12 | NTTN | AUUGCUCCUUACGAGGAGACAAUCAGUGGCUAACAGA | 1335 |
| 2 | T505_CasPhi.12 | NTTN | AUUGCUCCUUACGAGGAGACAGAAAUGGCGGCGUUUU | 1336 |
| 2 | T506_CasPhi.12 | NTTN | AUUGCUCCUUACGAGGAGACAUCAGUGGCUAACAGAA | 1337 |
| 2 | T507_CasPhi.12 | NTTN | AUUGCUCCUUACGAGGAGACGGAACAUGCUAAAUACA | 1338 |
| 2 | T508_CasPhi.12 | NTTN | AUUGCUCCUUACGAGGAGACCUGAGAAUUGGGAACAU | 1339 |
| 2 | T509_CasPhi.12 | NTTN | AUUGCUCCUUACGAGGAGACCUCAGAAAGACACAAAU | 1340 |
| 2 with | | NTTN | AUAGAUUGCUCCUUACGAGGAGACGUAUCUUACAGGAACUC | 1341 |
| L26R | | | | |
| 2 with | | NTTN | AUAGAUUGCUCCUUACGAGGAGACUGGUAUCUUACAGGAAC | 1342 |
| L26R | | | | |
| 2 with | | NTTN | AUAGAUUGCUCCUUACGAGGAGACUGGUAUCUUAmCmAmG | 1343 |
| L26R | | | | |

TABLE 8 provides illustrative sgRNA sequences that are useful in the compositions, systems and methods described herein, wherein.

TABLE 8
EXEMPLARY sgRNA SEQUENCES FOR USE IN SINGLE GUIDE SYSTEMS

| | Effector | | | |
|---|---|---|---|---|
| | Protein | | | SEQ |
| Composition | SEQ | | | ID |
| Name | ID NO. | PAM | sgRNA Sequence (5′→3′), shown as RNA | NO: |
| T01_CasM. | 3 | TTCG | UGGGGCAGUUGGUUGCCCUUAGCCUGAGGCAUUUAUUGCACUCGGGAAGUACCAUU | 1344 |
| 19952 | | | UCUCAGAAAUGGUACAUCCAACAAAUUGUAUUUAUCUUCAGCUUUUA | |
| T02_CasM. | 3 | TTCG | UGGGGCAGUUGGUUGCCCUUAGCCUGAGGCAUUUAUUGCACUCGGGAAGUACCAUU | 1345 |
| 19952 | | | UCUCAGAAAUGGUACAUCCAACAAAAAACAAAUCAAAGACUUUUUUA | |
| T03_CasM. | 3 | ATCG | UGGGGCAGUUGGUUGCCCUUAGCCUGAGGCAUUUAUUGCACUCGGGAAGUACCAUU | 1346 |
| 19952 | | | UCUCAGAAAUGGUACAUCCAACCCUGCAGGUAAAAGCAUAUGUUUUA | |
| T04_CasM. | 3 | GTCG | UGGGGCAGUUGGUUGCCCUUAGCCUGAGGCAUUUAUUGCACUCGGGAAGUACCAUU | 1347 |
| 19952 | | | UCUCAGAAAUGGUACAUCCAACCCCUACCUCUUUUUUCUGUCUUUUA | |
| T05_CasM. | 3 | ATCG | UGGGGCAGUUGGUUGCCCUUAGCCUGAGGCAUUUAUUGCACUCGGGAAGUACCAUU | 1348 |
| 19952 | | | UCUCAGAAAUGGUACAUCCAACAAUAAGUAAUGUGUAUGCUUUUUUA | |
| T06_CasM. | 3 | TTCG | UGGGGCAGUUGGUUGCCCUUAGCCUGAGGCAUUUAUUGCACUCGGGAAGUACCAUU | 1349 |
| 19952 | | | UCUCAGAAAUGGUACAUCCAACAUUAACACUUUGAAGAUAAUUUUUA | |
| T01_Cas14a. | 4 | TTTA | ACCGCUUCACCAAGUGCUGUCCCUUAGGGGAUUAGcACUUGAGUGAAGGUGGGCUGC | 1350 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACGCCUGGGUGACAGUGAGACUUUUUA | |
| T02_Cas14a. | 4 | TTTA | ACCGCUUCACCAAGUGCUGUCCCUUAGGGGAUUAGcACUUGAGUGAAGGUGGGCUGC | 1351 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACUUUUAAAAAGGUAUCUUUGAUUUUA | |
| T03_Cas14a. | 4 | TTTA | ACCGCUUCACCAAGUGCUGUCCCUUAGGGGAUUAGcACUUGAGUGAAGGUGGGCUGC | 1352 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACACGUGAUUUUCUGUUAAUAAUUUUA | |
| T04_Cas14a. | 4 | TTTA | ACCGCUUCACCAAGUGCUGUCCCUUAGGGGAUUAGcACUUGAGUGAAGGUGGGCUGC | 1353 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACCAUUAAACAUCAUUAAAUUAUUUUA | |
| T05_Cas14a. | 4 | TTTG | ACCGCUUCACCAAGUGCUGUCCCUUAGGGGAUUAGcACUUGAGUGAAGGUGGGCUGC | 1354 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACAUACUAACCUUGGUUUCUGUUUUUA | |
| T06_Cas14a. | 4 | TTTA | ACCGCUUCACCAAGUGCUGUCCCUUAGGGGAUUAGcACUUGAGUGAAGGUGGGCUGC | 1355 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACAAAAGGUAUCUUUGAUACUAUUUUA | |
| T07_Cas14a. | 4 | TTTA | ACCGCUUCACCAAGUGCUGUCCCUUAGGGGAUUAGcACUUGAGUGAAGGUGGGCUGC | 1356 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACUCAAAUGUAACCAGUAUUUUUUUUA | |
| T08_Cas14a. | 4 | TTTA | ACCGCUUCACCAAGUGCUGUCCCUUAGGGGAUUAGcACUUGAGUGAAGGUGGGCUGC | 1357 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACAUGUAAAGUUAUUAACAGAAUUUUA | |
| T09_Cas14a. | 4 | TTTA | ACCGCUUCACCAAGUGCUGUCCCUUAGGGGAUUAGcACUUGAGUGAAGGUGGGCUGC | 1358 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACUUUUCUUAGAGACAGAGUCUUUUUA | |
| T10_Cas14a. | 4 | TTTG | ACCGCUUCACCAAGUGCUGUCCCUUAGGGGAUUAGcACUUGAGUGAAGGUGGGCUGC | 1359 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACUAUAUUUAUUUUCUUAGAGAUUUUA | |
| T11_Cas14a. | 4 | TTTA | ACCGCUUCACCAAGUGCUGUCCCUUAGGGGAUUAGcACUUGAGUGAAGGUGGGCUGC | 1360 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACAAAUAAAAUACUGGUUACAUUUUUA | |
| T12_Cas14a. | 4 | TTTA | ACCGCUUCACCAAGUGCUGUCCCUUAGGGGAUUAGcACUUGAGUGAAGGUGGGCUGC | 1361 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACAUGAUGUUUAAUGUAAAGUUUUUUA | |
| T13_Cas14a. | 4 | TTTG | ACCGCUUCACCAAGUGCUGUCCCUUAGGGGAUUAGcACUUGAGUGAAGGUGGGCUGC | 1362 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACAUAAAAUUAUACCAUAGAUUUUUUA | |
| T14_Cas14a. | 4 | TTTG | ACCGCUUCACCAAGUGCUGUCCCUUAGGGGAUUAGcACUUGAGUGAAGGUGGGCUGC | 1363 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACAUGGCAAAUAUUAGUUUCUGUUUUA | |
| T15_Cas14a. | 4 | TTTA | ACCGCUUCACCAAGUGCUGUCCCUUAGGGGAUUAGcACUUGAGUGAAGGUGGGCUGC | 1364 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACAAUGUAACUUCCAAACGUUAUUUUA | |
| T16_Cas14a. | 4 | TTTA | ACCGCUUCACCAAGUGCUGUCCCUUAGGGGAUUAGcACUUGAGUGAAGGUGGGCUGC | 1365 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACAAAAAUUAUUCAUUGUGUUAUUUUA | |
| T17_Cas14a. | 4 | TTTA | ACCGCUUCACCAAGUGCUGUCCCUUAGGGGAUUAGcACUUGAGUGAAGGUGGGCUGC | 1366 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACUGUUGCUUAUUUAAAAAAUUUUUUA | |
| T18_Cas14a. | 4 | TTTG | ACCGCUUCACCAAGUGCUGUCCCUUAGGGGAUUAGcACUUGAGUGAAGGUGGGCUGC | 1367 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACCUUUUUUGAUGGCAAAUAUUUUUUA | |
| T19_Cas14a. | 4 | TTTA | ACCGCUUCACCAAGUGCUGUCCCUUAGGGGAUUAGcACUUGAGUGAAGGUGGGCUGC | 1368 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACACAGAAACUAAUAUUUGCCAUUUUA | |
| T20_Cas14a. | 4 | TTTA | ACCGCUUCACCAAGUGCUGUCCCUUAGGGGAUUAGcACUUGAGUGAAGGUGGGCUGC | 1369 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACAAAUGUCUCCUCCAGACUAGUUUUA | |
| T21_Cas14a. | 4 | TTTG | ACCGCUUCACCAAGUGCUGUCCCUUAGGGGAUUAGcACUUGAGUGAAGGUGGGCUGC | 1370 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACCCAUCAAAAAAGCAAAGAAUUUUUA | |
| T22_Cas14a. | 4 | TTTA | ACCGCUUCACCAAGUGCUGUCCCUUAGGGGAUUAGcACUUGAGUGAAGGUGGGCUGC | 1371 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACAAUAAGCAACAUAAAUGUGAUUUUA | |
| T23_Cas14a. | 4 | TTTA | ACCGCUUCACCAAGUGCUGUCCCUUAGGGGAUUAGcACUUGAGUGAAGGUGGGCUGC | 1372 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACUUUUUCCUUUUAUUCUAGUUUUUUA | |
| T24_Cas14a. | 4 | TTTA | ACCGCUUCACCAAGUGCUGUCCCUUAGGGGAUUAGcACUUGAGUGAAGGUGGGCUGC | 1373 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACUUCUAGUUGAAAGAAUUCAGUUUUA | |
| T25_Cas14a. | 4 | TTTA | ACCGCUUCACCAAGUGCUGUCCCUUAGGGGAUUAGcACUUGAGUGAAGGUGGGCUGC | 1374 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACCUACUAUAUAUUUAUUUUUCUUUUA | |
| T26_Cas14a. | 4 | TTTG | ACCGCUUCACCAAGUGCUGUCCCUUAGGGGAUUAGcACUUGAGUGAAGGUGGGCUGC | 1375 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACGAAGUUACAUUUAAAAUGUCUUUUA | |
| T27_Cas14a. | 4 | TTTA | ACCGCUUCACCAAGUGCUGUCCCUUAGGGGAUUAGcACUUGAGUGAAGGUGGGCUGC | 1376 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACUAUCCUUGAUUAUACUUAGGUUUUA | |
| T28_Cas14a. | 4 | TTTG | ACCGCUUCACCAAGUGCUGUCCCUUAGGGGAUUAGcACUUGAGUGAAGGUGGGCUGC | 1377 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACCAUUAAUUUAUAUCCUUGAUUUUUA | |
| T29_Cas14a. | 4 | TTTA | ACCGCUUCACCAAGUGCUGUCCCUUAGGGGAUUAGcACUUGAGUGAAGGUGGGCUGC | 1378 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACAAGAAAAACUUCUGCCAACUUUUUA | |
| T30_Cas14a. | 4 | TTTA | ACCGCUUCACCAAGUGCUGUCCCUUAGGGGAUUAGcACUUGAGUGAAGGUGGGCUGC | 1379 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACUCAUUUUUUCUCAUACCUUCUUUUA | |
| T31_Cas14a. | 4 | TTTG | ACCGCUUCACCAAGUGCUGUCCCUUAGGGGAUUAGcACUUGAGUGAAGGUGGGCUGC | 1380 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACUUAUUUGCAUUAAUUUAUAUUUUUA | |
| T32_Cas14a. | 4 | TTTG | ACCGCUUCACCAAGUGCUGUCCCUUAGGGGAUUAGcACUUGAGUGAAGGUGGGCUGC | 1381 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACCUGAGAGAGAAACAGUUGCCUUUUA | |
| T33_Cas14a. | 4 | TTTA | ACCGCUUCACCAAGUGCUGUCCCUUAGGGGAUUAGcACUUGAGUGAAGGUGGGCUGC | 1382 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACCUCUCCUAGACCAUUUCCCAUUUUA | |
| T34_Cas14a. | 4 | TTTA | ACCGCUUCACCAAGUGCUGUCCCUUAGGGGAUUAGcACUUGAGUGAAGGUGGGCUGC | 1383 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACAAAUGAAGAUUUUCCACCAAUUUUA | |
| T35_Cas14a. | 4 | TTTG | ACCGCUUCACCAAGUGCUGUCCCUUAGGGGAUUAGcACUUGAGUGAAGGUGGGCUGC | 1384 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACAGCCAAACUCUUAUUCAUGAUUUUA | |
| T36_Cas14a. | 4 | TTTG | ACCGCUUCACCAAGUGCUGUCCCUUAGGGGAUUAGcACUUGAGUGAAGGUGGGCUGC | 1385 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACGGUUUUUGCAAAAAGGAAAAUUUUA | |
| T37_Cas14a. | 4 | TTTA | ACCGCUUCACCAAGUGCUGUCCCUUAGGGGAUUAGcACUUGAGUGAAGGUGGGCUGC | 1386 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACGCAAUACAUGGUAGAAAAUGUUUUA | |
| T38_Cas14a. | 4 | TTTG | ACCGCUUCACCAAGUGCUGUCCCUUAGGGGAUUAGcACUUGAGUGAAGGUGGGCUGC | 1387 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACUUUAGCAAUACAUGGUAGAAUUUUA | |
| T39_Cas14a. | 4 | TTTA | ACCGCUUCACCAAGUGCUGUCCCUUAGGGGAUUAGcACUUGAGUGAAGGUGGGCUGC | 1388 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACAUUGAAGAGUAACAAUUUGAUUUUA | |
| T40_Cas14a. | 4 | TTTG | ACCGCUUCACCAAGUGCUGUCCCUUAGGGGAUUAGcACUUGAGUGAAGGUGGGCUGC | 1389 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACCAAAAAGGAAAAAAGAAGAAUUUUA | |
| T41_Cas14a. | 4 | TTTA | ACCGCUUCACCAAGUGCUGUCCCUUAGGGGAUUAGcACUUGAGUGAAGGUGGGCUGC | 1390 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACGCUUGUGUUUCUAAUUUUUCUUUUA | |
| T42_Cas14a. | 4 | TTTG | ACCGCUUCACCAAGUGCUGUCCCUUAGGGGAUUAGcACUUGAGUGAAGGUGGGCUGC | 1391 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACGCUCAAAUUGUUACUCUUCAUUUUA | |
| T43_Cas14a. | 4 | TTTG | ACCGCUUCACCAAGUGCUGUCCCUUAGGGGAUUAGcACUUGAGUGAAGGUGGGCUGC | 1392 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACACUUAUUGUUAUUGAAAUUGUUUUA | |
| T44_Cas14a. | 4 | TTTA | ACCGCUUCACCAAGUGCUGUCCCUUAGGGGAUUAGcACUUGAGUGAAGGUGGGCUGC | 1393 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACGCUCCUACUCAGACUGUUACUUUUA | |
| T45_Cas14a. | 4 | TTTG | ACCGCUUCACCAAGUGCUGUCCCUUAGGGGAUUAGcACUUGAGUGAAGGUGGGCUGC | 1394 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACCAAAAACCCAAAAUAUUUUAUUUUA | |
| T46_Cas14a. | 4 | TTTG | ACCGCUUCACCAAGUGCUGUCCCUUAGGGGAUUAGcACUUGAGUGAAGGUGGGCUGC | 1395 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACCACAGUCAAUAACACAAAGGUUUUA | |
| T47_Cas14a. | 4 | TTTG | ACCGCUUCACCAAGUGCUGUCCCUUAGGGGAUUAGcACUUGAGUGAAGGUGGGCUGC | 1396 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACAACAAAUAGCUAGAGCCAAAUUUUA | |
| T48_Cas14a. | 4 | TTTG | ACCGCUUCACCAAGUGCUGUCCCUUAGGGGAUUAGcACUUGAGUGAAGGUGGGCUGC | 1397 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACAAUCAGAGUCAAUUUCCAAGUUUUA | |
| T49_Cas14a. | 4 | TTTA | ACCGCUUCACCAAGUGCUGUCCCUUAGGGGAUUAGcACUUGAGUGAAGGUGGGCUGC | 1398 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACUCUCUAUCUUUAGAAUUGAAUUUUA | |
| T50_Cas14a. | 4 | TTTA | ACCGCUUCACCAAGUGCUGUCCCUUAGGGGAUUAGcACUUGAGUGAAGGUGGGCUGC | 1399 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACGAAUUGAAACAAAUUUUCUCUUUUA | |
| T51_Cas14a. | 4 | TTTG | ACCGCUUCACCAAGUGCUGUCCCUUAGGGGAUUAGcACUUGAGUGAAGGUGGGCUGC | 1400 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACGCUCUAGCUAUUUGUUCAAAUUUUA | |
| T52_Cas14a. | 4 | TTTG | ACCGCUUCACCAAGUGCUGUCCCUUAGGGGAUUAGcACUUGAGUGAAGGUGGGCUGC | 1401 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACUUUCAAUUCUAAAGAUAGAGUUUUA | |
| T53_Cas14a. | 4 | TTTG | ACCGCUUCACCAAGUGCUGUCCCUUAGGGGAUUAGcACUUGAGUGAAGGUGGGCUGC | 1402 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACUUCAAAAGUGCAACUAUGAAUUUUA | |
| T54_Cas14a. | 4 | TTTG | ACCGCUUCACCAAGUGCUGUCCCUUAGGGGAUUAGcACUUGAGUGAAGGUGGGCUGC | 1403 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACUGUUAUUGACUGUGCAAAAAUUUUA | |
| T55_Cas14a. | 4 | TTTA | ACCGCUUCACCAAGUGCUGUCCCUUAGGGGAUUAGcACUUGAGUGAAGGUGGGCUGC | 1404 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACGUGGGUAGAAUUUCUUUUAAUUUUA | |
| T56_Cas14a. | 4 | TTTG | ACCGCUUCACCAAGUGCUGUCCCUUAGGGGAUUAGcACUUGAGUGAAGGUGGGCUGC | 1405 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACGUGAAUAUAUUAUUGGAUUUUUUUA | |
| T57_Cas14a. | 4 | TTTA | ACCGCUUCACCAAGUGCUGUCCCUUAGGGGAUUAGcACUUGAGUGAAGGUGGGCUGC | 1406 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACACAGAAAAGCAUACACAUUAUUUUA | |
| T58_Cas14a. | 4 | TTTA | ACCGCUUCACCAAGUGCUGUCCCUUAGGGGAUUAGcACUUGAGUGAAGGUGGGCUGC | 1407 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACCUUCUAAAUUAACUUUAGUGUUUUA | |
| T59_Cas14a. | 4 | TTTG | ACCGCUUCACCAAGUGCUGUCCCUUAGGGGAUUAGcACUUGAGUGAAGGUGGGCUGC | 1408 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACAAGAUAAUUCAUGAACAUCUUUUUA | |
| T60_Cas14a. | 4 | TTTA | ACCGCUUCACCAAGUGCUGUCCCUUAGGGGAUUAGcACUUGAGUGAAGGUGGGCUGC | 1409 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACGCCAACCACCCUACAAAUAUUUUUA | |
| T61_Cas14a. | 4 | TTTA | ACCGCUUCACCAAGUGCUGUCCCUUAGGGGAUUAGcACUUGAGUGAAGGUGGGCUGC | 1410 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACAAGGAAUUAUAAUUAUUUUAUUUUA | |
| T62_Cas14a. | 4 | TTTA | ACCGCUUCACCAAGUGCUGUCCCUUAGGGGAUUAGcACUUGAGUGAAGGUGGGCUGC | 1411 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACGAAGUAAAAUAUAAUAGAAAUUUUA | |
| T63_Cas14a. | 4 | TTTG | ACCGCUUCACCAAGUGCUGUCCCUUAGGGGAUUAGcACUUGAGUGAAGGUGGGCUGC | 1412 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACUAGGGUGGUUGGCUAAAAUAUUUUA | |
| T64_Cas14a. | 4 | TTTA | ACCGCUUCACCAAGUGCUGUCCCUUAGGGGAUUAGcACUUGAGUGAAGGUGGGCUGC | 1413 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACAAAGAAAUUCUACCCACUAAUUUUA | |
| T65_Cas14a. | 4 | TTTA | ACCGCUUCACCAAGUGCUGUCCCUUAGGGGAUUAGcACUUGAGUGAAGGUGGGCUGC | 1414 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACCUGCUGUUGAUUAAUGGUUGUUUUA | |
| T66_Cas14a. | 4 | TTTA | ACCGCUUCACCAAGUGCUGUCCCUUAGGGGAUUAGcACUUGAGUGAAGGUGGGCUGC | 1415 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACAUGUUAGUGCCUUUCACCCUUUUUA | |
| T67_Cas14a. | 4 | TTTA | ACCGCUUCACCAAGUGCUGUCCCUUAGGGGAUUAGcACUUGAGUGAAGGUGGGCUGC | 1416 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACGUAUAUGAAAAAAAGAAAUAUUUUA | |
| T68_Cas14a. | 4 | TTTA | ACCGCUUCACCAAGUGCUGUCCCUUAGGGGAUUAGcACUUGAGUGAAGGUGGGCUGC | 1417 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACAGCUUUCUUUAGAAGAAUAUUUUUA | |
| T69_Cas14a. | 4 | TTTA | ACCGCUUCACCAAGUGCUGUCCCUUAGGGGAUUAGcACUUGAGUGAAGGUGGGCUGC | 1418 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACGAAGAAUAUUUCAUGAGAGAUUUUA | |
| T70_Cas14a. | 4 | TTTA | ACCGCUUCACCAAGUGCUGUCCCUUAGGGGAUUAGcACUUGAGUGAAGGUGGGCUGC | 1419 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACGCAGACUUUUUAAGCUUUCUUUUUA | |
| T71_Cas14a. | 4 | TTTA | ACCGCUUCACCAAGUGCUGUCCCUUAGGGGAUUAGcACUUGAGUGAAGGUGGGCUGC | 1420 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACUUUCUUUUUUUCAUAUACUAUUUUA | |
| T72_Cas14a. | 4 | TTTG | ACCGCUUCACCAAGUGCUGUCCCUUAGGGGAUUAGcACUUGAGUGAAGGUGGGCUGC | 1421 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACAGAAAAGAUUAAACAGUGUGUUUUA | |
| T73_Cas14a. | 4 | TTTA | ACCGCUUCACCAAGUGCUGUCCCUUAGGGGAUUAGcACUUGAGUGAAGGUGGGCUGC | 1422 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACUUUGAGAAAAGAUUAAACAGUUUUA | |
| T74_Cas14a. | 4 | TTTG | ACCGCUUCACCAAGUGCUGUCCCUUAGGGGAUUAGcACUUGAGUGAAGGUGGGCUGC | 1423 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACUUUUGCCUUUUUGGUAUCUUUUUUA | |
| T75_Cas14a. | 4 | TTTG | ACCGCUUCACCAAGUGCUGUCCCUUAGGGGAUUAGcACUUGAGUGAAGGUGGGCUGC | 1424 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACUAUUUCUUUCUUUGCCAGUAUUUUA | |
| T76_Cas14a. | 4 | TTTG | ACCGCUUCACCAAGUGCUGUCCCUUAGGGGAUUAGcACUUGAGUGAAGGUGGGCUGC | 1425 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACGUAUCUUACAGGAACUCCAGUUUUA | |
| T77_Cas14a. | 4 | TTTG | ACCGCUUCACCAAGUGCUGUCCCUUAGGGGAUUAGcACUUGAGUGAAGGUGGGCUGC | 1426 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACCCUUUUUGGUAUCUUACAGGUUUUA | |
| T78_Cas14a. | 4 | TTTG | ACCGCUUCACCAAGUGCUGUCCCUUAGGGGAUUAGcACUUGAGUGAAGGUGGGCUGC | 1427 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACCCAGUACAACUGCAUGUGGUUUUUA | |
| T79_Cas14a. | 4 | TTTG | ACCGCUUCACCAAGUGCUGUCCCUUAGGGGAUUAGcACUUGAGUGAAGGUGGGCUGC | 1428 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACGAUAUGGGCAUGUCAGUUUCUUUUA | |
| T80_Cas14a. | 4 | TTTA | ACCGCUUCACCAAGUGCUGUCCCUUAGGGGAUUAGcACUUGAGUGAAGGUGGGCUGC | 1429 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACAUCUUUUCUCAAAUAAAAAGUUUUA | |
| T81_Cas14a. | 4 | TTTA | ACCGCUUCACCAAGUGCUGUCCCUUAGGGGAUUAGcACUUGAGUGAAGGUGGGCUGC | 1430 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACAGAAGUUAAAGAGUCCAGAUUUUUA | |
| T82_Cas14a. | 4 | TTTG | ACCGCUUCACCAAGUGCUGUCCCUUAGGGGAUUAGcACUUGAGUGAAGGUGGGCUGC | 1431 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACAUUUGUUUUUUCGAAAUUGUUUUUA | |
| T83_Cas14a. | 4 | TTTG | ACCGCUUCACCAAGUGCUGUCCCUUAGGGGAUUAGcACUUGAGUGAAGGUGGGCUGC | 1432 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACACUGUUGUUGUCAUCAUUAUUUUUA | |
| T84_Cas14a. | 4 | TTTG | ACCGCUUCACCAAGUGCUGUCCCUUAGGGGAUUAGcACUUGAGUGAAGGUGGGCUGC | 1433 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACUUUUUUCGAAAUUGUAUUUAUUUUA | |
| T85_Cas14a. | 4 | TTTA | ACCGCUUCACCAAGUGCUGUCCCUUAGGGGAUUAGcACUUGAGUGAAGGUGGGCUGC | 1434 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACACUUCUUAAAGAUCAGGUUCUUUUA | |
| T86_Cas14a. | 4 | TTTA | ACCGCUUCACCAAGUGCUGUCCCUUAGGGGAUUAGcACUUGAGUGAAGGUGGGCUGC | 1435 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACUCUUCAGCACAUCUGGACUCUUUUA | |
| T87_Cas14a. | 4 | TTTG | ACCGCUUCACCAAGUGCUGUCCCUUAGGGGAUUAGcACUUGAGUGAAGGUGGGCUGC | 1436 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACCAGAGUUUAGUUUCAAGUAAUUUUA | |
| T88_Cas14a. | 4 | TTTA | ACCGCUUCACCAAGUGCUGUCCCUUAGGGGAUUAGcACUUGAGUGAAGGUGGGCUGC | 1437 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACGUUUCAAGUAACAAAUAAAAUUUUA | |
| T89_Cas14a. | 4 | TTTG | ACCGCUUCACCAAGUGCUGUCCCUUAGGGGAUUAGcACUUGAGUGAAGGUGGGCUGC | 1438 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACGUUAUACUGACAAAGAUAUCUUUUA | |
| T90_Cas14a. | 4 | TTTA | ACCGCUUCACCAAGUGCUGUCCCUUAGGGGAUUAGcACUUGAGUGAAGGUGGGCUGC | 1439 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACUUUGUUACUUGAAACUAAACUUUUA | |
| T91_Cas14a. | 4 | TTTG | ACCGCUUCACCAAGUGCUGUCCCUUAGGGGAUUAGcACUUGAGUGAAGGUGGGCUGC | 1440 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACUUACUUGAAACUAAACUCUGUUUUA | |
| T92_Cas14a. | 4 | TTTG | ACCGCUUCACCAAGUGCUGUCCCUUAGGGGAUUAGcACUUGAGUGAAGGUGGGCUGC | 1441 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACACAGAUCUGUUGAGAAAUGGUUUUA | |
| T93_Cas14a. | 4 | TTTA | ACCGCUUCACCAAGUGCUGUCCCUUAGGGGAUUAGcACUUGAGUGAAGGUGGGCUGC | 1442 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACCAUAAUCCAUCUAUUUUUCUUUUUA | |
| T94_Cas14a. | 4 | TTTA | ACCGCUUCACCAAGUGCUGUCCCUUAGGGGAUUAGcACUUGAGUGAAGGUGGGCUGC | 1443 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACCCUGCAGGCGAUUUGACAGAUUUUA | |
| T95_Cas14a. | 4 | TTTA | ACCGCUUCACCAAGUGCUGUCCCUUAGGGGAUUAGcACUUGAGUGAAGGUGGGCUGC | 1444 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACAAAUGUUGUGUGUACAUGCUUUUUA | |
| T96_Cas14a. | 4 | TTTG | ACCGCUUCACCAAGUGCUGUCCCUUAGGGGAUUAGcACUUGAGUGAAGGUGGGCUGC | 1445 |
| 1 | | | UUGCAUCAGCCUAAUGUCGAGAAGUGCUUUCUUCGGAAAGUAACCCUCGAAACAAA | |
| | | | UUCAUUUGAAAGAAUGAAGGAAUGCAACUCAGUAUAACCAAAAAAUAUUUUUA | |
| T97_Cas14a | 4 | TTTN | ACCGCUUCACCAAgUGCUGUCCCUUAGGGAUUAGcACUUGAGUGAAGGUGGGCUGCU | 1446 |
| 1 | | | UGCAUCAGCCUAAUGUCGAGAAGUGCUUUCUUCGGAAAGUAACCCUCGAAACAAAU | |
| | | | UCAUUUGAAAGAAUGAAGGAAUGCAACACAUUUCAUUCAACUGUUGC | |
| T98_Cas14a. | 4 | TTTN | ACCGCUUCACCAAgUGCUGUCCCUUAGGGAUUAGcACUUGAGUGAAGGUGGGCUGCU | 1447 |
| 1 | | | UGCAUCAGCCUAAUGUCGAGAAGUGCUUUCUUCGGAAAGUAACCCUCGAAACAAAU | |
| | | | UCAUUUGAAAGAAUGAAGGAAUGCAACGAUUGCAUCUACUGUAUAGG | |
| T99_Cas14a. | 4 | TTTN | ACCGCUUCACCAAgUGCUGUCCCUUAGGGAUUAGcACUUGAGUGAAGGUGGGCUGCU | 1448 |
| 1 | | | UGCAUCAGCCUAAUGUCGAGAAGUGCUUUCUUCGGAAAGUAACCCUCGAAACAAAU | |
| | | | UCAUUUGAAAGAAUGAAGGAAUGCAACUAACUUGAUCAAGCAGAGAA | |
| T100_Cas14a. | 4 | TTTN | ACCGCUUCACCAAgUGCUGUCCCUUAGGGAUUAGcACUUGAGUGAAGGUGGGCUGCU | 1449 |
| 1 | | | UGCAUCAGCCUAAUGUCGAGAAGUGCUUUCUUCGGAAAGUAACCCUCGAAACAAAU | |
| | | | UCAUUUGAAAGAAUGAAGGAAUGCAACGAGAUGGCAGUUUCCUUAGU | |
| T101_ | 4 | TTTN | ACCGCUUCACCAAgUGCUGUCCCUUAGGGAUUAGcACUUGAGUGAAGGUGGGCUGCU | 1450 |
| Cas14a.1 | | | UGCAUCAGCCUAAUGUCGAGAAGUGCUUUCUUCGGAAAGUAACCCUCGAAACAAAU | |
| | | | UCAUUUGAAAGAAUGAAGGAAUGCAACCCCUCAGCUCUUGAAGUAAA | |
| T102_ | 4 | TTTN | ACCGCUUCACCAAgUGCUGUCCCUUAGGGAUUAGcACUUGAGUGAAGGUGGGCUGCU | 1451 |
| Cas14a.1 | | | UGCAUCAGCCUAAUGUCGAGAAGUGCUUUCUUCGGAAAGUAACCCUCGAAACAAAU | |
| | | | UCAUUUGAAAGAAUGAAGGAAUGCAACCCGCCUUCCACUCAGAGCUC | |
| T103_ | 4 | TTTN | ACCGCUUCACCAAgUGCUGUCCCUUAGGGAUUAGcACUUGAGUGAAGGUGGGCUGCU | 1452 |
| Cas14a.1 | | | UGCAUCAGCCUAAUGUCGAGAAGUGCUUUCUUCGGAAAGUAACCCUCGAAACAAAU | |
| | | | UCAUUUGAAAGAAUGAAGGAAUGCAACCUUCAAGAGCUGAGGGCAAA | |
| T104_ | 4 | TTTN | ACCGCUUCACCAAgUGCUGUCCCUUAGGGAUUAGcACUUGAGUGAAGGUGGGCUGCU | 1453 |
| Cas14a.1 | | | UGCAUCAGCCUAAUGUCGAGAAGUGCUUUCUUCGGAAAGUAACCCUCGAAACAAAU | |
| | | | UCAUUUGAAAGAAUGAAGGAAUGCAACCAGACCUCCUGCCACCGCAG | |
| T105_ | 4 | TTTN | ACCGCUUCACCAAgUGCUGUCCCUUAGGGAUUAGcACUUGAGUGAAGGUGGGCUGCU | 1454 |
| Cas141a.1 | | | UGCAUCAGCCUAAUGUCGAGAAGUGCUUUCUUCGGAAAGUAACCCUCGAAACAAAU | |
| | | | UCAUUUGAAAGAAUGAAGGAAUGCAACCCGCUGCCCAAUGCCAUCCU | |
| T106_ | 4 | TTTN | ACCGCUUCACCAAgUGCUGUCCCUUAGGGAUUAGcACUUGAGUGAAGGUGGGCUGCU | 1455 |
| Cas14a.1 | | | UGCAUCAGCCUAAUGUCGAGAAGUGCUUUCUUCGGAAAGUAACCCUCGAAACAAAU | |
| | | | UCAUUUGAAAGAAUGAAGGAAUGCAACAGGAUUGCUGAAUUAUUUCU | |
| T107_ | 4 | TTTN | ACCGCUUCACCAAgUGCUGUCCCUUAGGGAUUAGcACUUGAGUGAAGGUGGGCUGCU | 1456 |
| Cas14a.1 | | | UGCAUCAGCCUAAUGUCGAGAAGUGCUUUCUUCGGAAAGUAACCCUCGAAACAAAU | |
| | | | UCAUUUGAAAGAAUGAAGGAAUGCAACGCAUGUUCCCAAUUCUCAGG | |
| T108_ | 4 | TTTN | ACCGCUUCACCAAgUGCUGUCCCUUAGGGAUUAGcACUUGAGUGAAGGUGGGCUGCU | 1457 |
| Cas14a.1 | | | UGCAUCAGCCUAAUGUCGAGAAGUGCUUUCUUCGGAAAGUAACCCUCGAAACAAAU | |
| | | | UCAUUUGAAAGAAUGAAGGAAUGCAACUAUCAUAAUGAAAACGCCGC | |
| T109_ | 4 | TTTN | ACCGCUUCACCAAgUGCUGUCCCUUAGGGAUUAGcACUUGAGUGAAGGUGGGCUGCU | 1458 |
| Cas14a.1 | | | UGCAUCAGCCUAAUGUCGAGAAGUGCUUUCUUCGGAAAGUAACCCUCGAAACAAAU | |
| | | | UCAUUUGAAAGAAUGAAGGAAUGCAACUGUCUUUCUGAGAAACUGUU | |
| T110_ | 4 | TTTN | ACCGCUUCACCAAgUGCUGUCCCUUAGGGAUUAGcACUUGAGUGAAGGUGGGCUGCU | 1459 |
| Cas14a.1 | | | UGCAUCAGCCUAAUGUCGAGAAGUGCUUUCUUCGGAAAGUAACCCUCGAAACAAAU | |
| | | | UCAUUUGAAAGAAUGAAGGAAUGCAACUAUUUAGCAUGUUCCCAAUU | |
| T111_ | 4 | TTTN | ACCGCUUCACCAAgUGCUGUCCCUUAGGGAUUAGcACUUGAGUGAAGGUGGGCUGCU | 1460 |
| Cas14a.1 | | | UGCAUCAGCCUAAUGUCGAGAAGUGCUUUCUUCGGAAAGUAACCCUCGAAACAAAU | |
| | | | UCAUUUGAAAGAAUGAAGGAAUGCAACAUCAGUGGCUAACAGAAGCU | |

TABLE 9 provides illustrative target nucleic acids that are useful in the compositions, systems and methods described herein.

TABLE 9
EXEMPLARY TARGET NUCLEIC ACIDS
Exemplary targets

DMD (also known as: BMD, CMD3B, MRX85, DXS142, DXS164,
DXS206, DXS230, DXS239, DXS268, DXS269, DXS270, DXS272)

TABLE 9.1 provides illustrative target nucleic acids that are useful in the compositions, systems and methods described herein.

TABLE 9.1
EXEMPLARY DMD SEQUENCES

| Description | Exemplary DMD Sequences |
|---|---|
| Dystrophin | MLWWEEVEDCYEREDVQKKTFTKWVNAQFSKFGKQHIENLFSDLQDGRRLLDLLEGLTGQKLPKEKGSTRVHALNNVNKALRVLQNN |
| Protein (Uniprot | NVDLVNIGSTDIVDGNHKLTLGLIWNIILHWQVKNVMKNIMAGLQQTNSEKILLSWVRQSTRNYPQVNVINFTTSWSDGLALNALIHSH |
| Accession No. | RPDLFDWNSVVCQQSATQRLEHAFNIARYQLGIEKLLDPEDVDTTYPDKKSILMYITSLFQVLPQQVSIEAIQEVEMLPRPPKVTKEEHFQ |
| P11532 | LHHQMHYSQQITVSLAQGYERTSSPKPRFKSYAYTQAAYVTTSDPTRSPFPSQHLEAPEDKSFGSSLMESEVNLDRYQTALEEVLSWLLS |
| (DMD_HUMAN))) | AEDTLQAQGEISNDVEVVKDQFHTHEGYMMDLTAHQGRVGNILQLGSKLIGTGKLSEDEETEVQEQMNLLNSRWECLRVASMEKQSN |
| SEQ ID NO: 1461 | LHRVLMDLQNQKLKELNDWLTKTEERTRKMEEEPLGPDLEDLKRQVQQHKVLQEDLEQEQVRVNSLTHMVVVVDESSGDHATAALE |
| | EQLKVLGDRWANICRWTEDRWVLLQDILLKWQRLTEEQCLFSAWLSEKEDAVNKIHTTGFKDQNEMLSSLQKLAVLKADLEKKKQSM |
| | GKLYSLKQDLLSTLKNKSVTQKTEAWLDNFARCWDNLVQKLEKSTAQISQAVTTTQPSLTQTTVMETVTTVTTREQILVKHAQEELPPP |
| | PPQKKRQITVDSEIRKRLDVDITELHSWITRSEAVLQSPEFAIFRKEGNFSDLKEKVNAIEREKAEKFRKLQDASRSAQALVEQMVNEGVN |
| | ADSIKQASEQLNSRWIEFCQLLSERLNWLEYQNNIIAFYNQLQQLEQMTTTAENWLKIQPTTPSEPTAIKSQLKICKDEVNRLSDLQPQIER |
| | LKIQSIALKEKGQGPMFLDADFVAFTNHFKQVFSDVQAREKELQTIFDTLPPMRYQETMSAIRTWVQQSETKLSIPQLSVTDYEIMEQRLG |
| | ELQALQSSLQEQQSGLYYLSTTVKEMSKKAPSEISRKYQSEFEEIEGRWKKLSSQLVEHCQKLEEQMNKLRKIQNHIQTLKKWMAEVDV |
| | FLKEEWPALGDSEILKKQLKQCRLLVSDIQTIQPSLNSVNEGGQKIKNEAEPEFASRLETELKELNTQWDHMCQQVYARKEALKGGLEK |
| | TVSLQKDLSEMHEWMTQAEEEYLERDFEYKTPDELQKAVEEMKRAKEEAQQKEAKVKLLTESVNSVIAQAPPVAQEALKKELETLTTN |
| | YQWLCTRLNGKCKTLEEVWACWHELLSYLEKANKWLNEVEFKLKTTENIPGGAEEISEVLDSLENLMRHSEDNPNQIRILAQTLTDGGV |
| | MDELINEELETFNSRWRELHEEAVRRQKLLEQSIQSAQETEKSLHLIQESLTFIDKQLAAYIADKVDAAQMPQEAQKIQSDLTSHEISLEE |
| | MKKHNQGKEAAQRVLSQIDVAQKKLQDVSMKFRLFQKPANFEQRLQESKMILDEVKMHLPALETKSVEQEVVQSQLNHCVNLYKSLS |
| | EVKSEVEMVIKTGRQIVQKKQTENPKELDERVTALKLHYNELGAKVTERKQQLEKCLKLSRKMRKEMNVLTEWLAATDMELTKRSAV |
| | EGMPSNLDSEVAWGKATQKEIEKQKVHLKSITEVGEALKTVLGKKETLVEDKLSLLNSNWIAVTSRAEEWLNLLLEYQKHMETFDQNV |
| | DHITKWIIQADTLLDESEKKKPQQKEDVLKRLKAELNDIRPKVDSTRDQAANLMANRGDHCRKLVEPQISELNHRFAAISHRIKTGKASIP |
| | LKELEQFNSDIQKLLEPLEAEIQQGVNLKEEDFNKDMNEDNEGTVKELLQRGDNLQQRITDERKREEIKIKQQLLQTKHNALKDLRSQRR |
| | KKALEISHQWYQYKRQADDLLKCLDDIEKKLASLPEPRDERKIKEIDRELQKKKEELNAVRRQAEGLSEDGAAMAVEPTQIQLSKRWRE |
| | IESKFAQFRRLNFAQIHTVREETMMVMTEDMPLEISYVPSTYLTEITHVSQALLEVEQLLNAPDLCAKDFEDLFKQEESLKNIKDSLQQSS |
| | GRIDIIHSKKTAALQSATPVERVKLQEALSQLDFQWEKVNKMYKDRQGRFDRSVEKWRRFHYDIKIFNQWLTEAEQFLRKTQIPENWEH |
| | AKYKWYLKELQDGIGQRQTVVRTLNATGEEIIQQSSKTDASILQEKLGSLNLRWQEVCKQLSDRKKRLEEQKNILSEFQRDLNEFVLWL |
| | EEADNIASIPLEPGKEQQLKEKLEQVKLLVEELPLRQGILKQLNETGGPVLVSAPISPEEQDKLENKLKQTNLQWIKVSRALPEKQGEIEAQ |
| | IKDLGQLEKKLEDLEEQLNHLLLWLSPIRNQLEIYNQPNQEGPFDVKETEIAVQAKQPDVEEILSKGQHLYKEKPATQPVKRKLEDLSSE |
| | WKAVNRLLQELRAKQPDLAPGLTTIGASPTQTVTLVTQPVVTKETAISKLEMPSSLMLEVPALADFNRAWTELTDWLSLLDQVIKSQRV |
| | MVGDLEDINEMIIKQKATMQDLEQRRPQLEELITAAQNLKNKTSNQEARTIITDRIERIQNQWDEVQEHLQNRRQQLNEMLKDSTQWLE |
| | AKEEAEQVLGQARAKLESWKEGPYTVDAIQKKITETKQLAKDLRQWQTNVDVANDLALKLLRDYSADDTRKVHMITENINASWRSIH |
| | KRVSEREAALEETHRLLQQFPLDLEKFLAWLTEAETTANVLQDATRKERLLEDSKGVKELMKQWQDLQGEIEAHTDVYHNLDENSQKI |
| | LRSLEGSDDAVLLQRRLDNMNFKWSELRKKSLNIRSHLEASSDQWKRLHLSLQELLVWLQLKDDELSRQAPIGGDFPAVQKQNDVHRA |
| | FKRELKTKEPVIMSTLETVRIFLTEQPLEGLEKLYQEPRELPPEERAQNVTRLLRKQAEEVNTEWEKLNLHSADWQRKIDETLERLQELQE |
| | ATDELDLKLRQAEVIKGSWQPVGDLLIDSLQDHLEKVKALRGEIAPLKENVSHVNDLARQLTTLGIQLSPYNLSTLEDLNTRWKLLQVA |
| | VEDRVRQLHEAHRDFGPASQHELSTSVQGPWERAISPNKVPYYINHETQTTCWDHPKMTELYQSLADLNNVRFSAYRTAMKLRRLQKA |
| | LCLDLLSLSAACDALDQHNLKQNDQPMDILQIINCLTTIYDRLEQEHNNLVNVPLCVDMCLNWLLNVYDTGRTGRIRVLSFKTGIISLCK |
| | AHLEDKYRYLFKQVASSTGFCDQRRLGLLLHDSIQIPRQLGEVASFGGSNIEPSVRSCFQFANNKPEIEAALFLDWMRLEPQSMVWLPVL |
| | HRVAAAETAKHQAKCNICKECPIIGFRYRSLKHFNYDICQSCFFSGRVAKGHKMHYPMVEYCTPTTSGEDVRDFAKVLKNKFRTKRYFA |
| | KHPRMGYLPVQTVLEGDNMETPVTLINFWPVDSAPASSPQLSHDDTHSRIEHYASRLAEMENSNGSYLNDSISPNESIDDEHLLIQHYCQS |
| | LNQDSPLSQPRSPAQILISLESEERGELERILADLEEENRNLQAEYDRLKQQHEHKGLSPLPSPPEMMPTSPQSPRDAELIAEAKLLRQHKG |
| | RLEARMQILEDHNKQLESQLHRLRQLLEQPQAEAKVNGTTVSSPSTSLQRSDSSQPMLLRVVGSQTSDSMGEEDLLSPPQDTSTGLEEVM |
| | EQLNNSFPSSRGRNTPGKPMREDTM |
| Dystrophin | ATCAGTTACTGTGTTGACTCACTCAGTGTTGGGATCACTCACTTTCCCCCTACAGGACTCAGATCTGGGAGGCAATTACCTTCGGAG |
| (NCBI | AAAAACGAATAGGAAAAACTGAAGTGTTACTTTTTTTAAAGCTGCTGAAGTTTGTTGGTTTCTCATTGTTTTTAAGCCTACTGGAGC |
| Reference | AATAAAGTTTGAAGAACTTTTACCAGGTTTTTTTTATCGCTGCCTTGATATACACTTTTCAAAATGCTTTGGTGGGAAGAAGTAGAG |
| Sequence No. | GACTGTTATGAAAGAGAAGATGTTCAAAAGAAAACATTCACAAAATGGGTAAATGCACAATTTTCTAAGTTTGGGAAGCAGCATA |
| NM_004006.3) | TTGAGAACCTCTTCAGTGACCTACAGGATGGGAGGCGCCTCCTAGACCTCCTCGAAGGCCTGACAGGGCAAAAACTGCCAAAAGA |
| SEQ ID NO: 1462 | AAAAGGATCCACAAGAGTTCATGCCCTGAACAATGTCAACAAGGCACTGCGGGTTTTGCAGAACAATAATGTTGATTTAGTGAATA |
| | TTGGAAGTACTGACATCGTAGATGGAAATCATAAACTGACTCTTGGTTTGATTTGGAATATAATCCTCCACTGGCAGGTCAAAAAT |
| | GTAATGAAAAATATCATGGCTGGATTGCAACAAACCAACAGTGAAAAGATTCTCCTGAGCTGGGTCCGACAATCAACTCGTAATTA |
| | TCCACAGGTTAATGTAATCAACTTCACCACCAGCTGGTCTGATGGCCTGGCTTTGAATGCTCTCATCCATAGTCATAGGCCAGACCT |
| | ATTTGACTGGAATAGTGTGGTTTGCCAGCAGTCAGCCACACAACGACTGGAACATGCATTCAACATCGCCAGATATCAATTAGGCA |
| | TAGAGAAACTACTCGATCCTGAAGATGTTGATACCACCTATCCAGATAAGAAGTCCATCTTAATGTACATCACATCACTCTTCCAA |
| | GTTTTGCCTCAACAAGTGAGCATTGAAGCCATCCAGGAAGTGGAAATGTTGCCAAGGCCACCTAAAGTGACTAAAGAAGAACATT |
| | TTCAGTTACATCATCAAATGCACTATTCTCAACAGATCACGGTCAGTCTAGCACAGGGATATGAGAGAACTTCTTCCCCTAAGCCTC |
| | GATTCAAGAGCTATGCCTACACACAGGCTGCTTATGTCACCACCTCTGACCCTACACGGAGCCCATTTCCTTCACAGCATTTGGAAG |
| | CTCCTGAAGACAAGTCATTTGGCAGTTCATTGATGGAGAGTGAAGTAAACCTGGACCGTTATCAAACAGCTTTAGAAGAAGTATTA |
| | TCGTGGCTTCTTTCTGCTGAGGACACATTGCAAGCACAAGGAGAGATTTCTAATGATGTGGAAGTGGTGAAAGACCAGTTTCATAC |
| | TCATGAGGGGTACATGATGGATTTGACAGCCCATCAGGGCCGGGTTGGTAATATTCTACAATTGGGAAGTAAGCTGATTGGAACAG |
| | GAAAATTATCAGAAGATGAAGAAACTGAAGTACAAGAGCAGATGAATCTCCTAAATTCAAGATGGGAATGCCTCAGGGTAGCTAG |
| | CATGGAAAAACAAAGCAATTTACATAGAGTTTTAATGGATCTCCAGAATCAGAAACTGAAAGAGTTGAATGACTGGCTAACAAAA |
| | ACAGAAGAAAGAACAAGGAAAATGGAGGAAGAGCCTCTTGGACCTGATCTTGAAGACCTAAAACGCCAAGTACAACAACATAAG |
| | GTGCTTCAAGAAGATCTAGAACAAGAACAAGTCAGGGTCAATTCTCTCACTCACATGGTGGTGGTAGTTGATGAATCTAGTGGAGA |
| | TCACGCAACTGCTGCTTTGGAAGAACAACTTAAGGTATTGGGAGATCGATGGGCAAACATCTGTAGATGGACAGAAGACCGCTGG |
| | GTTCTTTTACAAGACATCCTTCTCAAATGGCAACGTCTTACTGAAGAACAGTGCCTTTTTAGTGCATGGCTTTCAGAAAAAGAAGAT |
| | GCAGTGAACAAGATTCACACAACTGGCTTTAAAGATCAAAATGAAATGTTATCAAGTCTTCAAAAACTGGCCGTTTTAAAAGCGGA |
| | TCTAGAAAAGAAAAAGCAATCCATGGGCAAACTGTATTCACTCAAACAAGATCTTCTTTCAACACTGAAGAATAAGTCAGTGACCC |
| | AGAAGACGGAAGCATGGCTGGATAACTTTGCCCGGTGTTGGGATAATTTAGTCCAAAAACTTGAAAAGAGTACAGCACAGATTTC |
| | ACAGGCTGTCACCACCACTCAGCCATCACTAACACAGACAACTGTAATGGAAACAGTAACTACGGTGACCACAAGGGAACAGATC |
| | CTGGTAAAGCATGCTCAAGAGGAACTTCCACCACCACCTCCCCAAAAGAAGAGGCAGATTACTGTGGATTCTGAAATTAGGAAAA |
| | GGTTGGATGTTGATATAACTGAACTTCACAGCTGGATTACTCGCTCAGAAGCTGTGTTGCAGAGTCCTGAATTTGCAATCTTTCGGA |
| | AGGAAGGCAACTTCTCAGACTTAAAAGAAAAAGTCAATGCCATAGAGCGAGAAAAAGCTGAGAAGTTCAGAAAACTGCAAGATG |
| | CCAGCAGATCAGCTCAGGCCCTGGTGGAACAGATGGTGAATGAGGGTGTTAATGCAGATAGCATCAAACAAGCCTCAGAACAACT |
| | GAACAGCCGGTGGATCGAATTCTGCCAGTTGCTAAGTGAGAGACTTAACTGGCTGGAGTATCAGAACAACATCATCGCTTTCTATA |
| | ATCAGCTACAACAATTGGAGCAGATGACAACTACTGCTGAAAACTGGTTGAAAATCCAACCCACCACCCCATCAGAGCCAACAGC |
| | AATTAAAAGTCAGTTAAAAATTTGTAAGGATGAAGTCAACCGGCTATCAGATCTTCAACCTCAAATTGAACGATTAAAAATTCAAA |
| | GCATAGCCCTGAAAGAGAAAGGACAAGGACCCATGTTCCTGGATGCAGACTTTGTGGCCTTTACAAATCATTTTAAGCAAGTCTTT |
| | TCTGATGTGCAGGCCAGAGAGAAAGAGCTACAGACAATTTTTGACACTTTGCCACCAATGCGCTATCAGGAGACCATGAGTGCCAT |
| | CAGGACATGGGTCCAGCAGTCAGAAACCAAACTCTCCATACCTCAACTTAGTGTCACCGACTATGAAATCATGGAGCAGAGACTC |
| | GGGGAATTGCAGGCTTTACAAAGTTCTCTGCAAGAGCAACAAAGTGGCCTATACTATCTCAGCACCACTGTGAAAGAGATGTCGA |
| | AGAAAGCGCCCTCTGAAATTAGCCGGAAATATCAATCAGAATTTGAAGAAATTGAGGGACGCTGGAAGAAGCTCTCCTCCCAGCT |
| | GGTTGAGCATTGTCAAAAGCTAGAGGAGCAAATGAATAAACTCCGAAAAATTCAGAATCACATACAAACCCTGAAGAAATGGATG |
| | GCTGAAGTTGATGTTTTTCTGAAGGAGGAATGGCCTGCCCTTGGGGATTCAGAAATTCTAAAAAAGCAGCTGAAACAGTGCAGACT |
| | TTTAGTCAGTGATATTCAGACAATTCAGCCCAGTCTAAACAGTGTCAATGAAGGTGGGCAGAAGATAAAGAATGAAGCAGAGCCA |
| | GAGTTTGCTTCGAGACTTGAGACAGAACTCAAAGAACTTAACACTCAGTGGGATCACATGTGCCAACAGGTCTATGCCAGAAAGG |
| | AGGCCTTGAAGGGAGGTTTGGAGAAAACTGTAAGCCTCCAGAAAGATCTATCAGAGATGCACGAATGGATGACACAAGCTGAAGA |
| | AGAGTATCTTGAGAGAGATTTTGAATATAAAACTCCAGATGAATTACAGAAAGCAGTTGAAGAGATGAAGAGAGCTAAAGAAGA |
| | GGCCCAACAAAAAGAAGCGAAAGTGAAACTCCTTACTGAGTCTGTAAATAGTGTCATAGCTCAAGCTCCACCTGTAGCACAAGAG |
| | GCCTTAAAAAAGGAACTTGAAACTCTAACCACCAACTACCAGTGGCTCTGCACTAGGCTGAATGGGAAATGCAAGACTTTGGAAG |
| | AAGTTTGGGCATGTTGGCATGAGTTATTGTCATACTTGGAGAAAGCAAACAAGTGGCTAAATGAAGTAGAATTTAAACTTAAAACC |
| | ACTGAAAACATTCCTGGCGGAGCTGAGGAAATCTCTGAGGTGCTAGATTCACTTGAAAATTTGATGCGACATTCAGAGGATAACCC |
| | AAATCAGATTCGCATATTGGCACAGACCCTAACAGATGGCGGAGTCATGGATGAGCTAATCAATGAGGAACTTGAGACATTTAATT |
| | CTCGTTGGAGGGAACTACATGAAGAGGCTGTAAGGAGGCAAAAGTTGCTTGAACAGAGCATCCAGTCTGCCCAGGAGACTGAAAA |
| | ATCCTTACACTTAATCCAGGAGTCCCTCACATTCATTGACAAGCAGTTGGCAGCTTATATTGCAGACAAGGTGGACGCAGCTCAAA |
| | TGCCTCAGGAAGCCCAGAAAATCCAATCTGATTTGACAAGTCATGAGATCAGTTTAGAAGAAATGAAGAAACATAATCAGGGGAA |
| | GGAGGCTGCCCAAAGAGTCCTGTCTCAGATTGATGTTGCACAGAAAAAATTACAAGATGTCTCCATGAAGTTTCGATTATTCCAGA |
| | AACCAGCCAATTTTGAGCAGCGTCTACAAGAAAGTAAGATGATTTTAGATGAAGTGAAGATGCACTTGCCTGCATTGGAAACAAA |
| | GAGTGTGGAACAGGAAGTAGTACAGTCACAGCTAAATCATTGTGTGAACTTGTATAAAAGTCTGAGTGAAGTGAAGTCTGAAGTG |
| | GAAATGGTGATAAAGACTGGACGTCAGATTGTACAGAAAAAGCAGACGGAAAATCCCAAAGAACTTGATGAAAGAGTAACAGCT |
| | TTGAAATTGCATTATAATGAGCTGGGAGCAAAGGTAACAGAAAGAAAGCAACAGTTGGAGAAATGCTTGAAATTGTCCCGTAAGA |
| | TGCGAAAGGAAATGAATGTCTTGACAGAATGGCTGGCAGCTACAGATATGGAATTGACAAAGAGATCAGCAGTTGAAGGAATGCC |
| | TAGTAATTTGGATTCTGAAGTTGCCTGGGGAAAGGCTACTCAAAAAGAGATTGAGAAACAGAAGGTGCACCTGAAGAGTATCACA |
| | GAGGTAGGAGAGGCCTTGAAAACAGTTTTGGGCAAGAAGGAGACGTTGGTGGAAGATAAACTCAGTCTTCTGAATAGTAACTGGA |
| | TAGCTGTCACCTCCCGAGCAGAAGAGTGGTTAAATCTTTTGTTGGAATACCAGAAACACATGGAAACTTTTGACCAGAATGTGGAC |
| | CACATCACAAAGTGGATCATTCAGGCTGACACACTTTTGGATGAATCAGAGAAAAAGAAACCCCAGCAAAAAGAAGACGTGCTTA |
| | AGCGTTTAAAGGCAGAACTGAATGACATACGCCCAAAGGTGGACTCTACACGTGACCAAGCAGCAAACTTGATGGCAAACCGCGG |
| | TGACCACTGCAGGAAATTAGTAGAGCCCCAAATCTCAGAGCTCAACCATCGATTTGCAGCCATTTCACACAGAATTAAGACTGGAA |
| | AGGCCTCCATTCCTTTGAAGGAATTGGAGCAGTTTAACTCAGATATACAAAAATTGCTTGAACCACTGGAGGCTGAAATTCAGCAG |
| | GGGGTGAATCTGAAAGAGGAAGACTTCAATAAAGATATGAATGAAGACAATGAGGGTACTGTAAAAGAATTGTTGCAAAGAGGA |
| | GACAACTTACAACAAAGAATCACAGATGAGAGAAAGCGAGAGGAAATAAAGATAAAACAGCAGCTGTTACAGACAAAACATAAT |
| | GCTCTCAAGGATTTGAGGTCTCAAAGAAGAAAAAAGGCTCTAGAAATTTCTCATCAGTGGTATCAGTACAAGAGGCAGGCTGATG |
| | ATCTCCTGAAATGCTTGGATGACATTGAAAAAAAATTAGCCAGCCTACCTGAGCCCAGAGATGAAAGGAAAATAAAGGAAATTGA |
| | TCGGGAATTGCAGAAGAAGAAAGAGGAGCTGAATGCAGTGCGTAGGCAAGCTGAGGGCTTGTCTGAGGATGGGGCCGCAATGGC |
| | AGTGGAGCCAACTCAGATCCAGCTCAGCAAGCGCTGGCGGGAAATTGAGAGCAAATTTGCTCAGTTTCGAAGACTCAACTTTGCAC |
| | AAATTCACACTGTCCGTGAAGAAACGATGATGGTGATGACTGAAGACATGCCTTTGGAAATTTCTTATGTGCCTTCTACTTATTTGA |
| | CTGAAATCACTCATGTCTCACAAGCCCTATTAGAAGTGGAACAACTTCTCAATGCTCCTGACCTCTGTGCTAAGGACTTTGAAGATC |
| | TCTTTAAGCAAGAGGAGTCTCTGAAGAATATAAAAGATAGTCTACAACAAAGCTCAGGTCGGATTGACATTATTCATAGCAAGAA |
| | GACAGCAGCATTGCAAAGTGCAACGCCTGTGGAAAGGGTGAAGCTACAGGAAGCTCTCTCCCAGCTTGATTTCCAATGGGAAAAA |
| | GTTAACAAAATGTACAAGGACCGACAAGGGCGATTTGACAGATCTGTTGAGAAATGGCGGCGTTTTCATTATGATATAAAGATATT |
| | TAATCAGTGGCTAACAGAAGCTGAACAGTTTCTCAGAAAGACACAAATTCCTGAGAATTGGGAACATGCTAAATACAAATGGTAT |
| | CTTAAGGAACTCCAGGATGGCATTGGGCAGCGGCAAACTGTTGTCAGAACATTGAATGCAACTGGGGAAGAAATAATTCAGCAAT |
| | CCTCAAAAACAGATGCCAGTATTCTACAGGAAAAATTGGGAAGCCTGAATCTGCGGTGGCAGGAGGTCTGCAAACAGCTGTCAGA |
| | CAGAAAAAAGAGGCTAGAAGAACAAAAGAATATCTTGTCAGAATTTCAAAGAGATTTAAATGAATTTGTTTTATGGTTGGAGGAA |
| | GCAGATAACATTGCTAGTATCCCACTTGAACCTGGAAAAGAGCAGCAACTAAAAGAAAAGCTTGAGCAAGTCAAGTTACTGGTGG |
| | AAGAGTTGCCCCTGCGCCAGGGAATTCTCAAACAATTAAATGAAACTGGAGGACCCGTGCTTGTAAGTGCTCCCATAAGCCCAGA |
| | AGAGCAAGATAAACTTGAAAATAAGCTCAAGCAGACAAATCTCCAGTGGATAAAGGTTTCCAGAGCTTTACCTGAGAAACAAGGA |
| | GAAATTGAAGCTCAAATAAAAGACCTTGGGCAGCTTGAAAAAAAGCTTGAAGACCTTGAAGAGCAGTTAAATCATCTGCTGCTGT |
| | GGTTATCTCCTATTAGGAATCAGTTGGAAATTTATAACCAACCAAACCAAGAAGGACCATTTGACGTTAAGGAAACTGAAATAGCA |
| | GTTCAAGCTAAACAACCGGATGTGGAAGAGATTTTGTCTAAAGGGCAGCATTTGTACAAGGAAAAACCAGCCACTCAGCCAGTGA |
| | AGAGGAAGTTAGAAGATCTGAGCTCTGAGTGGAAGGCGGTAAACCGTTTACTTCAAGAGCTGAGGGCAAAGCAGCCTGACCTAGC |
| | TCCTGGACTGACCACTATTGGAGCCTCTCCTACTCAGACTGTTACTCTGGTGACACAACCTGTGGTTACTAAGGAAACTGCCATCTC |
| | CAAACTAGAAATGCCATCTTCCTTGATGTTGGAGGTACCTGCTCTGGCAGATTTCAACCGGGCTTGGACAGAACTTACCGACTGGC |
| | TTTCTCTGCTTGATCAAGTTATAAAATCACAGAGGGTGATGGTGGGTGACCTTGAGGATATCAACGAGATGATCATCAAGCAGAAG |
| | GCAACAATGCAGGATTTGGAACAGAGGCGTCCCCAGTTGGAAGAACTCATTACCGCTGCCCAAAATTTGAAAAACAAGACCAGCA |
| | ATCAAGAGGCTAGAACAATCATTACGGATCGAATTGAAAGAATTCAGAATCAGTGGGATGAAGTACAAGAACACCTTCAGAACCG |
| | GAGGCAACAGTTGAATGAAATGTTAAAGGATTCAACACAATGGCTGGAAGCTAAGGAAGAAGCTGAGCAGGTCTTAGGACAGGC |
| | CAGAGCCAAGCTTGAGTCATGGAAGGAGGGTCCCTATACAGTAGATGCAATCCAAAAGAAAATCACAGAAACCAAGCAGTTGGCC |
| | AAAGACCTCCGCCAGTGGCAGACAAATGTAGATGTGGCAAATGACTTGGCCCTGAAACTTCTCCGGGATTATTCTGCAGATGATAC |
| | CAGAAAAGTCCACATGATAACAGAGAATATCAATGCCTCTTGGAGAAGCATTCATAAAAGGGTGAGTGAGCGAGAGGCTGCTTTG |
| | GAAGAAACTCATAGATTACTGCAACAGTTCCCCCTGGACCTGGAAAAGTTTCTTGCCTGGCTTACAGAAGCTGAAACAACTGCCAA |
| | TGTCCTACAGGATGCTACCCGTAAGGAAAGGCTCCTAGAAGACTCCAAGGGAGTAAAAGAGCTGATGAAACAATGGCAAGACCTC |
| | CAAGGTGAAATTGAAGCTCACACAGATGTTTATCACAACCTGGATGAAAACAGCCAAAAAATCCTGAGATCCCTGGAAGGTTCCG |
| | ATGATGCAGTCCTGTTACAAAGACGTTTGGATAACATGAACTTCAAGTGGAGTGAACTTCGGAAAAAGTCTCTCAACATTAGGTCC |
| | CATTTGGAAGCCAGTTCTGACCAGTGGAAGCGTCTGCACCTTTCTCTGCAGGAACTTCTGGTGTGGCTACAGCTGAAAGATGATGA |
| | ATTAAGCCGGCAGGCACCTATTGGAGGCGACTTTCCAGCAGTTCAGAAGCAGAACGATGTACATAGGGCCTTCAAGAGGGAATTG |
| | AAAACTAAAGAACCTGTAATCATGAGTACTCTTGAGACTGTACGAATATTTCTGACAGAGCAGCCTTTGGAAGGACTAGAGAAACT |
| | CTACCAGGAGCCCAGAGAGCTGCCTCCTGAGGAGAGAGCCCAGAATGTCACTCGGCTTCTACGAAAGCAGGCTGAGGAGGTCAAT |
| | ACTGAGTGGGAAAAATTGAACCTGCACTCCGCTGACTGGCAGAGAAAAATAGATGAGACCCTTGAAAGACTCCGGGAACTTCAAG |
| | AGGCCACGGATGAGCTGGACCTCAAGCTGCGCCAAGCTGAGGTGATCAAGGGATCCTGGCAGCCCGTGGGCGATCTCCTCATTGA |
| | CTCTCTCCAAGATCACCTCGAGAAAGTCAAGGCACTTCGAGGAGAAATTGCGCCTCTGAAAGAGAACGTGAGCCACGTCAATGAC |
| | CTTGCTCGCCAGCTTACCACTTTGGGCATTCAGCTCTCACCGTATAACCTCAGCACTCTGGAAGACCTGAACACCAGATGGAAGCTT |
| | CTGCAGGTGGCCGTCGAGGACCGAGTCAGGCAGCTGCATGAAGCCCACAGGGACTTTGGTCCAGCATCTCAGCACTTTCTTTCCAC |
| | GTCTGTCCAGGGTCCCTGGGAGAGAGCCATCTCGCCAAACAAAGTGCCCTACTATATCAACCACGAGACTCAAACAACTTGCTGGG |
| | ACCATCCCAAAATGACAGAGCTCTACCAGTCTTTAGCTGACCTGAATAATGTCAGATTCTCAGCTTATAGGACTGCCATGAAACTC |
| | CGAAGACTGCAGAAGGCCCTTTGCTTGGATCTCTTGAGCCTGTCAGCTGCATGTGATGCCTTGGACCAGCACAACCTCAAGCAAAA |
| | TGACCAGCCCATGGATATCCTGCAGATTATTAATTGTTTGACCACTATTTATGACCGCCTGGAGCAAGAGCACAACAATTTGGTCA |
| | ACGTCCCTCTCTGCGTGGATATGTGTCTGAACTGGCTGCTGAATGTTTATGATACGGGACGAACAGGGAGGATCCGTGTCCTGTCTT |
| | TTAAAACTGGCATCATTTCCCTGTGTAAAGCACATTTGGAAGACAAGTACAGATACCTTTTCAAGCAAGTGGCAAGTTCAACAGGA |
| | TTTTGTGACCAGCGCAGGCTGGGCCTCCTTCTGCATGATTCTATCCAAATTCCAAGACAGTTGGGTGAAGTTGCATCCTTTGGGGGC |
| | AGTAACATTGAGCCAAGTGTCCGGAGCTGCTTCCAATTTGCTAATAATAAGCCAGAGATCGAAGCGGCCCTCTTCCTAGACTGGAT |
| | GAGACTGGAACCCCAGTCCATGGTGTGGCTGCCCGTCCTGCACAGAGTGGCTGCTGCAGAAACTGCCAAGCATCAGGCCAAATGT |
| | AACATCTGCAAAGAGTGTCCAATCATTGGATTCAGGTACAGGAGTCTAAAGCACTTTAATTATGACATCTGCCAAAGCTGCTTTTTT |
| | TCTGGTCGAGTTGCAAAAGGCCATAAAATGCACTATCCCATGGTGGAATATTGCACTCCGACTACATCAGGAGAAGATGTTCGAGA |
| | CTTTGCCAAGGTACTAAAAAACAAATTTCGAACCAAAAGGTATTTTGCGAAGCATCCCCGAATGGGCTACCTGCCAGTGCAGACTG |
| | TCTTAGAGGGGGACAACATGGAAACTCCCGTTACTCTGATCAACTTCTGGCCAGTAGATTCTGCGCCTGCCTCGTCCCCTCAGCTTT |
| | CACACGATGATACTCATTCACGCATTGAACATTATGCTAGCAGGCTAGCAGAAATGGAAAACAGCAATGGATCTTATCTAAATGAT |
| | AGCATCTCTCCTAATGAGAGCATAGATGATGAACATTTGTTAATCCAGCATTACTGCCAAAGTTTGAACCAGGACTCCCCCCTGAG |
| | CCAGCCTCGTAGTCCTGCCCAGATCTTGATTTCCTTAGAGAGTGAGGAAAGAGGGGAGCTAGAGAGAATCCTAGCAGATCTTGAG |
| | GAAGAAAACAGGAATCTGCAAGCAGAATATGACCGTCTAAAGCAGCAGCACGAACATAAAGGCCTGTCCCCACTGCCGTCCCCTC |
| | CTGAAATGATGCCCACCTCTCCCCAGAGTCCCCGGGATGCTGAGCTCATTGCTGAGGCCAAGCTACTGCGTCAACACAAAGGCCGC |
| | CTGGAAGCCAGGATGCAAATCCTGGAAGACCACAATAAACAGCTGGAGTCACAGTTACACAGGCTAAGGCAGCTGCTGGAGCAAC |
| | CCCAGGCAGAGGCCAAAGTGAATGGCACAACGGTGTCCTCTCCTTCTACCTCTCTACAGAGGTCCGACAGCAGTCAGCCTATGCTG |
| | CTCCGAGTGGTTGGCAGTCAAACTTCGGACTCCATGGGTGAGGAAGATCTTCTCAGTCCTCCCCAGGACACAAGCACAGGGTTAGA |
| | GGAGGTGATGGAGCAACTCAACAACTCCTTCCCTAGTTCAAGAGGAAGAAATACCCCTGGAAAGCCAATGAGAGAGGACACAATG |
| | TAGGAAGTCTTTTCCACATGGCAGATGATTTGGGCAGAGCGATGGAGTCCTTAGTATCAGTCATGACAGATGAAGAAGGAGCAGA |
| | ATAAATGTTTTACAACTCCTGATTCCCGCATGGTTTTTATAATATTCATACAACAAAGAGGATTAGACAGTAAGAGTTTACAAGAA |
| | ATAAATCTATATTTTTGTGAAGGGTAGTGGTATTATACTGTAGATTTCAGTAGTTTCTAAGTCTGTTATTGTTTTGTTAACAATGGCA |
| | GGTTTTACACGTCTATGCAATTGTACAAAAAAGTTATAAGAAAACTACATGTAAAATCTTGATAGCTAAATAACTTGCCATTTCTTT |
| | ATATGGAACGCATTTTGGGTTGTTTAAAAATTTATAACAGTTATAAAGAAAGATTGTAAACTAAAGTGTGCTTTATAAAAAAAAGT |
| | TGTTTATAAAAACCCCTAAAAACAAAACAAACACACACACACACACATACACACACACACACAAAACTTTGAGGCAGCGCATTGT |
| | TTTGCATCCTTTTGGCGTGATATCCATATGAAATTCATGGCTTTTTCTTTTTTTGCATATTAAAGATAAGACTTCCTCTACCACCACA |
| | CCAAATGACTACTACACACTGCTCATTTGAGAACTGTCAGCTGAGTGGGGCAGGCTTGAGTTTTCATTTCATATATCTATATGTCTA |
| | TAAGTATATAAATACTATAGTTATATAGATAAAGAGATACGAATTTCTATAGACTGACTTTTTCCATTTTTTAAATGTTCATGTCAC |
| | ATCCTAATAGAAAGAAATTACTTCTAGTCAGTCATCCAGGCTTACCTGCTTGGTCTAGAATGGATTTTTCCCGGAGCCGGAAGCCA |
| | GGAGGAAACTACACCACACTAAAACATTGTCTACAGCTCCAGATGTTTCTCATTTTAAACAACTTTCCACTGACAACGAAAGTAAA |
| | GTAAAGTATTGGATTTTTTTAAAGGGAACATGTGAATGAATACACAGGACTTATTATATCAGAGTGAGTAATCGGTTGGTTGGTTG |
| | ATTGATTGATTGATTGATACATTCAGCTTCCTGCTGCTAGCAATGCCACGATTTAGATTTAATGATGCTTCAGTGGAAATCAATCAG |
| | AAGGTATTCTGACCTTGTGAACATCAGAAGGTATTTTTTAACTCCCAAGCAGTAGCAGGACGATGATAGGGCTGGAGGGCTATGGA |
| | TTCCCAGCCCATCCCTGTGAAGGAGTAGGCCACTCTTTAAGTGAAGGATTGGATGATTGTTCATAATACATAAAGTTCTCTGTAATT |
| | ACAACTAAATTATTATGCCCTCTTCTCACAGTCAAAAGGAACTGGGTGGTTTGGTTTTTGTTGCTTTTTTAGATTTATTGTCCCATGT |
| | GGGATGAGTTTTTAAATGCCACAAGACATAATTTAAAATAAATAAACTTTGGGAAAAGGTGTAAAACAGTAGCCCCATCACATTTG |
| | TGATACTGACAGGTATCAACCCAGAAGCCCATGAACTGTGTTTCCATCCTTTGCATTTCTCTGCGAGTAGTTCCACACAGGTTTGTA |
| | AGTAAGTAAGAAAGAAGGCAAATTGATTCAAATGTTACAAAAAAACCCTTCTTGGTGGATTAGACAGGTTAAATATATAAACAAA |
| | CAAACAAAAATTGCTCAAAAAAGAGGAGAAAAGCTCAAGAGGAAAAGCTAAGGACTGGTAGGAAAAAGCTTTACTCTTTCATGCC |
| | ATTTTATTTCTTTTTGATTTTTAAATCATTCATTCAATAGATACCACCGTGTGACCTATAATTTTGCAAATCTGTTACCTCTGACATC |
| | AAGTGTAATTAGCTTTTGGAGAGTGGGCTGACATCAAGTGTAATTAGCTTTTGGAGAGTGGGTTTTGTCCATTATTAATAATTAATT |
| | AATTAACATCAAACACGGCTTCTCATGCTATTTCTACCTCACTTTGGTTTTGGGGTGTTCCTGATAATTGTGCACACCTGAGTTCACA |
| | GCTTCACCACTTGTCCATTGCGTTATTTTCTTTTTCCTTTATAATTCTTTCTTTTTCCTTCATAATTTTCAAAAGAAAACCCAAAGCTC |
| | TAAGGTAACAAATTACCAAATTACATGAAGATTTGGTTTTTGTCTTGCATTTTTTTCCTTTATGTGACGCTGGACCTTTTCTTTACCC |
| | AAGGATTTTTAAAACTCAGATTTAAAACAAGGGGTTACTTTACATCCTACTAAGAAGTTTAAGTAAGTAAGTTTCATTCTAAAATC |
| | AGAGGTAAATAGAGTGCATAAATAATTTTGTTTTAATCTTTTTGTTTTTCTTTTAGACACATTAGCTCTGGAGTGAGTCTGTCATAAT |
| | ATTTGAACAAAAATTGAGAGCTTTATTGCTGCATTTTAAGCATAATTAATTTGGACATTATTTCGTGTTGTGTTCTTTATAACCACCA |
| | AGTATTAAACTGTAAATCATAATGTAACTGAAGCATAAACATCACATGGCATGTTTTGTCATTGTTTTCAGGTACTGAGTTCTTACT |
| | TGAGTATCATAATATATTGTGTTTTAACACCAACACTGTAACATTTACGAATTATTTTTTTAAACTTCAGTTTTACTGCATTTTCACA |
| | ACATATCAGACTTCACCAAATATATGCCTTACTATTGTATTATAGTACTGCTTTACTGTGTATCTCAATAAAGCACGCAGTTATGTT |
| | ACAAAAAA |

TABLE 9.2
CERTAIN EXEMPLARY EXONS

| Exon No. | Start | End |
|---|---|---|
| 1 | 33,211,549 | 33,211,282 |
| 2 | 33,020,200 | 33,020,139 |
| 3 | 32,849,820 | 32,849,728 |
| 4 | 32,844,860 | 32,844,783 |
| 5 | 32,823,387 | 32,823,295 |
| 6 | 32,816,640 | 32,816,468 |
| 7 | 32,809,611 | 32,809,493 |
| 8 | 32,699,293 | 32,699,112 |
| 9 | 32,697,998 | 32,697,870 |
| 10 | 32,645,152 | 32,644,964 |
| 11 | 32,644,313 | 32,644,132 |
| 12 | 32,614,453 | 32,614,303 |
| 13 | 32,595,876 | 32,595,757 |
| 14 | 32,573,846 | 32,573,745 |
| 15 | 32,573,637 | 32,573,530 |
| 16 | 32,565,881 | 32,565,702 |
| 17 | 32,545,334 | 32,545,159 |
| 18 | 32,518,131 | 32,518,008 |
| 19 | 32,501,842 | 32,501,755 |
| 20 | 32,491,518 | 32,491,277 |
| 21 | 32,485,099 | 32,484,919 |
| 22 | 32,472,309 | 32,472,164 |
| 23 | 32,468,710 | 32,468,498 |
| 24 | 32,464,699 | 32,464,586 |
| 25 | 32,463,594 | 32,463,439 |
| 26 | 32,454,832 | 32,454,662 |
| 27 | 32,448,638 | 32,448,456 |
| 28 | 32,441,314 | 32,441,180 |
| 29 | 32,438,390 | 32,438,241 |
| 30 | 32,411,913 | 32,411,752 |
| 31 | 32,390,181 | 32,390,071 |
| 32 | 32,389,674 | 32,389,501 |
| 33 | 32,386,465 | 32,386,310 |
| 34 | 32,380,680 | 32,380,510 |
| 35 | 32,365,199 | 32,365,020 |
| 36 | 32,364,710 | 32,364,582 |
| 37 | 32,362,958 | 32,362,788 |
| 38 | 32,348,528 | 32,348,406 |
| 39 | 32,346,080 | 32,345,943 |
| 40 | 32,343,286 | 32,343,134 |
| 41 | 32,342,282 | 32,342,100 |
| 42 | 32,310,276 | 32,310,082 |
| 43 | 32,287,701 | 32,287,529 |
| 44 | 32,217,063 | 32,216,916 |
| 45 | 31,968,514 | 31,968,339 |
| 46 | 31,932,227 | 31,932,080 |
| 47 | 31,929,745 | 31,929,596 |
| 48 | 31,875,373 | 31,875,188 |
| 49 | 31,836,819 | 31,836,718 |
| 50 | 31,820,083 | 31,819,975 |
| 51 | 31,774,192 | 31,773,960 |
| 52 | 31,729,748 | 31,729,631 |
| 53 | 31,679,586 | 31,679,375 |
| 54 | 31,658,144 | 31,657,990 |
| 55 | 31,627,862 | 31,627,673 |
| 56 | 31,507,453 | 31,507,281 |
| 57 | 31,496,944 | 31,496,788 |
| 58 | 31,479,103 | 31,478,983 |
| 59 | 31,478,374 | 31,478,106 |
| 60 | 31,444,627 | 31,444,481 |
| 61 | 31,348,634 | 31,348,556 |
| 62 | 31,323,658 | 31,323,598 |
| 63 | 31,261,016 | 31,260,955 |
| 64 | 31,223,121 | 31,223,047 |
| 65 | 31,209,699 | 31,209,498 |
| 66 | 31,206,667 | 31,206,582 |
| 67 | 31,204,118 | 31,203,961 |
| 68 | 31,182,904 | 31,182,738 |
| 69 | 31,180,481 | 31,180,370 |
| 70 | 31,178,805 | 31,178,669 |
| 71 | 31,177,970 | 31,177,932 |
| 72 | 31,173,604 | 31,173,539 |
| 73 | 31,172,413 | 31,172,348 |
| 74 | 31,169,601 | 31,169,443 |
| 75 | 31,147,518 | 31,147,275 |
| 76 | 31,146,414 | 31,146,291 |
| 77 | 31,134,194 | 31,134,102 |
| 78 | 31,126,673 | 31,126,642 |
| 79 | 31,121,930 | 31,119,222 |

TABLE 9.3
CERTAIN EXEMPLARY GENOMIC EXON SEQUENCES

| Exon | | SEQ |
|---|---|---|
| No. | Exon Sequence | ID NO: |
| 44 | CTTAAGATACCATTTGTATTTAGCATGTTCCCAATTCTCAGGAAT | 1463 |
| | TTGTGTCTTTCTGAGAAACTGTTCAGCTTCTGTTAGCCACTGATT | |
| | AAATATCTTTATATCATAATGAAAACGCCGCCATTTCTCAACAG | |
| | ATCTGTCAAATCGC | |
| 45 | GAACTCCAGGATGGCATTGGGCAGCGGCAAACTGTTGTCAGAA | 1464 |
| | CATTGAATGCAACTGGGGAAGAAATAATTCAGCAATCCTCAAA | |
| | AACAGATGCCAGTATTCTACAGGAAAAATTGGGAAGCCTGAAT | |
| | CTGCGGTGGCAGGAGGTCTGCAAACAGCTGTCAGACAGAAAAA | |
| | AGAG | |
| 50 | AGGAAGTTAGAAGATCTGAGCTCTGAGTGGAAGGCGGTAAACC | 1465 |
| | GTTTACTTCAAGAGCTGAGGGCAAAGCAGCCTGACCTAGCTCCT | |
| | GGACTGACCACTATTGGAGCCT | |
| 51 | CTCCTACTCAGACTGTTACTCTGGTGACACAACCTGTGGTTACTA | 1466 |
| | AGGAAACTGCCATCTCCAAACTAGAAATGCCATCTTCCTTGATG | |
| | TTGGAGGTACCTGCTCTGGCAGATTTCAACCGGGCTTGGACAGA | |
| | ACTTACCGACTGGCTTTCTCTGCTTGATCAAGTTATAAAATCACA | |
| | GAGGGTGATGGTGGGTGACCTTGAGGATATCAACGAGATGATC | |
| | ATCAAGCAGAAG | |
| 53 | TTGAAAGAATTCAGAATCAGTGGGATGAAGTACAAGAACACCT | 1467 |
| | TCAGAACCGGAGGCAACAGTTGAATGAAATGTTAAAGGATTCA | |
| | ACACAATGGCTGGAAGCTAAGGAAGAAGCTGAGCAGGTCTTAG | |
| | GACAGGCCAGAGCCAAGCTTGAGTCATGGAAGGAGGGTCCCTA | |
| | TACAGTAGATGCAATCCAAAAGAAAATCACAGAAACCAAG | |

TABLE 9.4
CERTAIN EXEMPLARY MUTATIONS

| Exemplary Mutation Expression | Exemplary Target Gene Region |
|---|---|
| DMD exon 48-50 deletions and DMD nonsense mutation in exon 51 | DMD exon 51 |
| DMD exon 45-52 deletions | DMD exon 53 |
| DMD exon 44 deletion | DMD exon 45 |
| DMD nonsense mutation in exon 23 | DMD exon 23 |
| DMD exon 48-50 deletions DMD nonsense mutation in exon 51 | DMD exon 51 |
| DMD nonsense mutation in exon 23 | DMD intron 22 and 23 |
| DMD exon 46-51 deletions DMD exon 46-47 deletions | DMD intron 44 and 55 |
| DMD nonsense mutation in exon 23 | DMD intron 22 and 23 |
| DMD nonsense mutation in exon 23 | DMD exon 23 |
| DMD point mutation in intron 47, exon 51 | DMD exon 47A, exon 51 |
| DMD exon 44 deletion | DMD splice site of exon 43 or exon 45 |
| DMD nonsense mutation in exon 53 | DMD splice acceptor site of exon 53 |
| DMD exon 50 deletion | DMD splice acceptor site of exon 51 |
| DMD exon 50 deletion | DMD splice acceptor site of exon 51 |
| DMD nonsense mutation in exon 23 | DMD exon 23 |
| DMD nonsense mutation in exon 23 | DMD exon 23 |
| DMD nonsense mutation in exon 53 | DMD exon 53 |
| DMD exon 44 deletion | DMD exon 44 |
| DMD exon 7 skipping | DMD splice acceptor site of intron 6 and exon 7 boundary |
| DMD nonsense mutation in exon 23 | DMD exon 23 |
| DMD exon 51 deletion | DMD splice site of exon 50 |
| DMD nonsense mutation in exon 20 | DMD exon 20 |
| DMD exon 45-52 deletions | UTRN A, B promoter |
| DMD nonsense mutation in exon 23 | Lama1 promoter |
| DMD nonsense mutation in exon 23 | klotho and Utrn |
| DMD exon 46-51 deletions | 3′ UTR of UTRN inhibitory miRNA target region |
| epigenetic dysregulation of DUX4 | DUX4 promoter or DUX4 exon 1 |

TABLE 10 provides illustrative diseases, disorders and syndromes for compositions, systems and methods described herein.

TABLE 10
DISEASES AND SYNDROMES
Exemplary Diseases and Syndromes

Muscular Dystrophy (MD); Muscular Dystrophy, Duchenne Type (DMD); Dilated Cardiomyopathy (DCM) Type 3B; Muscular Dystrophy; Muscular
Dystrophy, Becker Type (BMD); Dystrophinopathies; Familial Isolated Dilated Cardiomyopathy; Dilated Cardiomyopathy; Myopathy;
Colorectal Cancer; Isolated Elevated Serum Creatine Phosphokinase Levels; Atrial Standstill 1; Creatine Phosphokinase, Elevated Serum;
Neuromuscular Disease; Atrial Heart Septal Defect; Arrhythmogenic Right Ventricular Cardiomyopathy; Heart Disease; Glycerol Kinase
Deficiency; Non-Syndromic X-Linked Intellectual Disability; Respiratory Failure; Rhabdomyosarcoma; Miyoshi Muscular Dystrophy;
Scoliosis; Facioscapulohumeral Muscular Dystrophy 1; Ptosis; Hypertrophic Cardiomyopathy; Schizophrenia; Myositis; Autism;
Myocarditis; Muscular Dystrophy, Limb-Girdle, Autosomal Recessive 2; Adrenal Hypoplasia, Congenital; Autosomal Recessive Limb-Girdle
Muscular Dystrophy; Restrictive Cardiomyopathy; Walker-Warburg Syndrome; Muscular Dystrophy, Congenital, Lmna-Related; Centronuclear
Myopathy; Cataract; Spinal Muscular Atrophy; Long Qt Syndrome; Emery-Dreifuss Muscular Dystrophy; Retinitis Pigmentosa; Malignant
Hyperthermia; Pectus Excavatum; Brugada Syndrome; Myoglobinuria; Muscular Dystrophy- Dystroglycanopathy, Type a, 4; Muscle
Hypertrophy; Cardiomyopathy, Familial Hypertrophic, 1; Batten-Turner Congenital Myopathy; Bethlem Myopathy 1; Eye Disease; Aland
Island Eye Disease; Glycogen Storage Disease II; Left Ventricular Noncompaction; Glycogen Storage Disease; Brody Myopathy;
Myofibrillar Myopathy; Beckwith-Wiedemann Syndrome; Polyglucosan Body Myopathy 1 with or Without Immunodeficiency; Interatrial
Communication; Congenital Fiber-Type Disproportion; Chromosome Xp21 Deletion Syndrome; Multiple Pterygium Syndrome, Escobar Variant;
Lissencephaly; Rigid Spine Muscular Dystrophy 1; Hypertrophic Pyloric Stenosis; Progressive Muscular Dystrophy; X-Linked Recessive
Disease; Muscular Disease; Disease of Mental Health; Exophthalmos; Gas Gangrene; Symptomatic Form of Muscular Dystrophy of Duchenne
and Becker in Female Carriers; Muscular Atrophy; Qualitative or Quantitative Defects of Dystrophin; Muscular Dystrophy, Congenital
Merosin-Deficient, 1a; Autosomal Recessive Limb-Girdle Muscular Dystrophy Type 2c; Mcleod Syndrome; Muscular Dystrophy, Duchenne and
Becker Type; Autosomal Recessive Limb-Girdle Muscular Dystrophy Type 2d; Ullrich Congenital Muscular Dystrophy 1; Nr0b1-Related
Adrenal Hypoplasia Congenita; Waardenburg Syndrome, Type 4b; Nonaka Myopathy; Intrinsic Cardiomyopathy; Autosomal Recessive Limb-
Girdle Muscular Dystrophy Type 2b; Myotonic Dystrophy 1; Muscular Dystrophy, Limb-Girdle, Autosomal Recessive 6; Emery-Dreifuss
Muscular Dystrophy 2, Autosomal Dominant; Muscular Dystrophy, Limb-Girdle, Autosomal Recessive 7; Myopathy, Myofibrillar, 3; Myopathy,
Myofibrillar, 5; Myopathy, Myofibrillar, 1; Peripheral Nervous System Disease; Muscle Eye Brain Disease; Cardiomyopathy, Familial
Hypertrophic, 4; Microcolon; Hemophagocytic Lymphohistiocytosis, Familial, 1; Autosomal Recessive Limb-Girdle Muscular Dystrophy
Type 2a; Tibial Muscular Dystrophy; Congenital Muscular Dystrophy-Dystroglycanopathy Type a; Bone Structure Disease; Autosomal
Recessive Limb-Girdle Muscular Dystrophy Type 2f; Immunodeficiency 26; Oculomedin; Cardioneuromyopathy with Hyaline Masses and
Nemaline Rods; Keratosis Follicularis Spinulosa Decalvans, X-Linked; Myoglobinuria, Recurrent; Muscular Dystrophy-Dystroglycanopathy;
X-Linked Monogenic Disease; Muscle Tissue Disease; Fundus Dystrophy; Interstitial Myocarditis; Localized Lipodystrophy;
Extracardiac Rhabdomyoma; Cytoplasmic Body Myopathy; Autosomal Dominant Distal Myopathy; Reducing Body Myopathy; Cobblestone
Lissencephaly; Multiple Sclerosis; Barrett Esophagus; Gastric Cancer, Hereditary Diffuse; Colorectal Cancer 12; Polyposis Syndrome,
Hereditary Mixed, 1; Small Intestine Adenocarcinoma; Esophageal Cancer; Esophagus Squamous Cell Carcinoma; Cardiomyopathy, Dilated,
1b; Limb-Girdle Muscular Dystrophy; 48, xyyy; 48, xxxy; 48, xxyy; Alacrima, Achalasia, and Mental Retardation Syndrome; 49, xyyyy; 49,
xxxxx; 49, xxxxy; 47, xyy; Lmna-Related Dilated Cardiomyopathy; Lung Combined Type Small Cell Carcinoma; Lung Occult Small Cell
Carcinoma; Lung Non-Squamous Non-Small Cell Carcinoma; Cardiomyopathy, Dilated, 1a; Cardiomyopathy, Dilated, 1h; Cardiac Conduction
Defect; Meningioma, Familial; Congestive Heart Failure; Myotonic Dystrophy; Breast Cancer; Severe Combined Immunodeficiency;
Dysphagia; Fibrosis of Extraocular Muscles, Congenital, 1; Muscular Dystrophy, Limb-Girdle, Autosomal Recessive 5; Autism Spectrum
Disorder; Polykaryocytosis Inducer; Progressive Familial Heart Block; Heart Conduction Disease; Relapsing-Remitting Multiple
Sclerosis; Colon Adenocarcinoma; Glioblastoma; Encephalopathy, Progressive, Early-Onset, with Episodic Rhabdomyolysis; Metabolic
Crises, Recurrent, with Rhabdomyolysis, Cardiac Arrhythmias, and Neurodegeneration; Attention Deficit-Hyperactivity Disorder;
Neuroretinitis; Retinitis; Learning Disability; Secondary Progressive Multiple Sclerosis; Myotonia Congenita, Autosomal Recessive;
Endomyocardial Fibrosis; Pik3ca-Related Overgrowth Syndrome; Genetic Neuromuscular Disease; Short Stature-Obesity Syndrome;
Hypoadrenocorticism, Familial; Cleft Palate, Isolated; Osteoporosis; Bone Mineral Density Quantitative Trait Locus 8; Bone Mineral
Density Quantitative Trait Locus 15; Gonadal Dysgenesis; Turner Syndrome; Hypoxia; Phenylketonuria; Brugada Syndrome 1; Neuronal
Migration Disorders; Cardiac Arrest; Hypotonia; Amyotrophic Lateral Sclerosis 1; Glioma Susceptibility 1; Lateral Sclerosis; Corneal
Edema; Polymyositis; Sleep Disorder; Cleft Lip; Muscular Dystrophy, Limb-Girdle, Autosomal Recessive 1; Glioma Susceptibility 9;
Glioma Susceptibility 2; Glioma Susceptibility 3; Pilocytic Astrocytoma; Diarrhea; Hemophilia; Chronic Granulomatous Disease; Cleft
Lip with or Without Cleft Palate; Type 2 Diabetes Mellitus; Triiodothyronine Receptor Auxiliary Protein; Macroglossia; Melanoma;
Orofaciodigital Syndrome I; Orofaciodigital Syndrome; Aging; Rapidly Involuting Congenital Hemangioma; Sensorineural Hearing Loss;
Yemenite Deaf-Blind Hypopigmentation Syndrome; Toxic Encephalopathy; West Syndrome; Gastrointestinal Stromal Tumor; Osteogenic
Sarcoma; Skeletal Muscle Disease; Intracranial Meningioma; Muscular Dystrophy-Dystroglycanopathy, Type C, 5; Ataxia, Combined
Cerebellar and Peripheral, with Hearing Loss and Diabetes Mellitus; Branchiootic Syndrome 1; Deafness, X-Linked 3; Secretory
Meningioma; Lymphoplasmacyte-Rich Meningioma; Factor Viii Deficiency; Hemophilia a; Dermatomyositis; Calpain-3-Related Limb-Girdle
Muscular Dystrophy R1; Qualitative or Quantitative Defects of Alpha-Dystroglycan; Congenital Muscular Dystrophy Due to
Dystroglycanopathy; Growth Hormone Deficiency; Cleft Lip/palate; Parkinsonism; Microcephaly; Cerebral Palsy; Osteomalacia; Bosma
Arhinia Microphthalmia Syndrome; Intraocular Pressure Quantitative Trait Locus; Combined Immunodeficiency; Maple Syrup Urine Disease;
Papillomatosis, Confluent and Reticulated; Peutz-Jeghers Syndrome; Rippling Muscle Disease 2; Muscular Dystrophy-Dystroglycanopathy,
Type B, 5; Nonsyndromic 46, xx Testicular Disorders of Sex Development; Hand Skill, Relative; Orofacial Cleft; Retinal Detachment;
Constipation; Sarcoma; Spindle Cell Sarcoma; Premature Menopause; Sleep Apnea; Dysferlinopathy; Qualitative or Quantitative Defects
of Dysferlin; Autonomic Dysfunction; Graft-Versus-Host Disease; Microphthalmia, Syndromic 10; Chondroblastoma; Bone Mineral Density
Quantitative Trait Locus 3; Leukemia, Acute Lymphoblastic; Methane Production; Ischemia; Idiopathic Scoliosis; Alcohol Dependence;
Premature Ovarian Failure 1; Demyelinating Disease; Qualitative or Quantitative Defects of Sarcoglycan; Mitral Valve Insufficiency;
Myopathy, X-Linked, with Excessive Autophagy; Mucositis; Inclusion Body Myositis; Dystonia; Bone Resorption Disease; Body Mass Index
Quantitative Trait Locus 1; Neuritis; Cystic Fibrosis; Polycystic Kidney Disease; Charcot-Marie-Tooth Disease; Myasthenia Gravis;
Helix Syndrome; Hyperinsulinism; Lipid Metabolism Disorder; Tooth Disease; Lung Disease; Muscular Dystrophy, Limb-Girdle, Autosomal
Recessive 3; Miyoshi Muscular Dystrophy 1; Pseudohyperkalemia, Familial, 2, Due to Red Cell Leak; Urinary Tract Infection; Early-
Onset Generalized Limb-Onset Dystonia; Multinucleated Neurons, Anhydramnios, Renal Dysplasia, Cerebellar Hypoplasia, and
Hydranencephaly; Agenesis of Corpus Callosum, Cardiac, Ocular, and Genital Syndrome; Pneumothorax; Proteasome-Associated
Autoinflammatory Syndrome 1; Pancreatic Cancer; Gastric Cancer; Neuromyelitis Optica; Open-Angle Glaucoma; Disease by Infectious
Agent; Resting Heart Rate, Variation in; Poliomyelitis; Childhood Type Dermatomyositis; Progressive Multifocal Leukoencephalopathy;
Swallowing Disorders; Premature Aging; Rickets; Hirschsprung Disease, Cardiac Defects, and Autonomic Dysfunction; Optic Neuritis;
Progressive Muscular Atrophy; Spinal Muscular Atrophy, Type Ii; Glucose Intolerance; Nephrolithiasis; Hypogonadism; Motor Neuron
Disease; Congenital Muscular Dystrophy Type la; Autoimmune Disease; Atrioventricular Block; Glucocorticoid-Induced Osteoporosis;
Epilepsy; Back Pain; Fragile X Syndrome; B-Lymphoblastic Leukemia/lymphoma; Mitochondrial Myopathy; Dyslexia; Ataxia-Telangiectasia;
Obsessive-Compulsive Disorder; Torticollis; Proteinuria, Chronic Benign; Pulmonary Fibrosis; Myotonia; Metabolic Acidosis; Brittle
Bone Disorder; Scoliosis, Isolated 1; Vascular Disease; Bilirubin Metabolic Disorder; Night Blindness; Chromosomal Triplication;
Dentinogenesis Imperfecta Type 2; Astigmatism; Severe Acute Respiratory Syndrome; Telangiectasis; Skin Disease; Microphthalmia;
Myopathy, Distal, with Anterior Tibial Onset; Hyperhomocysteinemia; Congenital Stationary Night Blindness; Hypothyroidism; Mitral
Valve Disease; Leiomyosarcoma; Nemaline Myopathy; Distal Muscular Dystrophy with Anterior Tibial Onset; Diabetes Mellitus; Influenza;
Herpes Simplex; Juvenile Rheumatoid Arthritis; Central Sleep Apnea; Homocysteinemia; Mitochondrial Disorders; Nervous System
Disease; Keratoconus; Nasopharyngitis; Glaucoma, Primary Open Angle; Central Centrifugal Cicatricial Alopecia; Anxiety; Dermatitis,
Atopic; Aphasia; Sexual Disorder; Acute Cystitis; Dermatitis; Kidney Disease; Tetanus; Myopia; Hypokalemia; Spinal Cord Injury;
Cyanide Poisoning; Cardiogenic Shock; Huntington Disease; Spinal Muscular Atrophy, Type I; Colitis; Down Syndrome; Hair Whorl;
Achondroplasia; Apnea, Obstructive Sleep; Barth Syndrome; Autosomal Recessive Disease; Hepatitis B; Movement Disease; Hemosiderosis;
Hepatitis; Gastric Dilatation; Teratoma; Gastroparesis; Progressive Familial Heart Block, Type Ia; Chondroma; Neurofibromatosis;
Hyperthyroidism; Enchondroma; Pulmonary Embolism; Hypoglycemia; Rare Hereditary Hemochromatosis; Paresthesia; Charcot-Marie-Tooth
Disease, Demyelinating, Type 1a; Keratitis, Hereditary; Insulin-Like Growth Factor I; Drug Allergy; Body Mass Index Quantitative
Trait Locus 8; Body Mass Index Quantitative Trait Locus 14; Body Mass Index Quantitative Trait Locus 18; Body Mass Index
Quantitative Trait Locus 7; Body Mass Index Quantitative Trait Locus 4; Body Mass Index Quantitative Trait Locus 10; Orthostatic
Intolerance; Body Mass Index Quantitative Trait Locus 12; Vitreoretinopathy, Neovascular Inflammatory; Abetalipoproteinemia; Body
Mass Index Quantitative Trait Locus 11; Body Mass Index Quantitative Trait Locus 9; Ichthyosis, X-Linked; Lymphoma; Ocular Albinism;
Cervical Dystonia; Uveitis; Hydrocephalus; Liver Cirrhosis; Acute Myocarditis; Skin Carcinoma; Ichthyosis; Hypertensive Heart Disease;
Hypogonadotropic Hypogonadism; Hepatocellular Carcinoma; Body Mass Index Quantitative Trait Locus 19; Pulmonary Hypertension;
Albinism; B-Cell Lymphoma; Allergic Encephalomyelitis; Cytokine Deficiency; Vitreoretinopathy; Prader-Willi Syndrome;
Oculopharyngeal Muscular Dystrophy; Neurodegeneration with Brain Iron Accumulation 2a; Spondylometaphyseal Dysplasia, Sedaghatian
Type; Aspiration Pneumonia; Human Immunodeficiency Virus Type 1; Arcus Corneae; Taqi Polymorphism; Inflammatory Bowel Disease;
Epidermolysis Bullosa; Temporal Lobe Epilepsy; Blepharospasm; Corneal Neovascularization; Neuroaxonal Dystrophy; Neurodevelopmental,
Jaw, Eye, and Digital Syndrome; Blistering, Acantholytic, of Oral and Laryngeal Mucosa; Collagen Vi-Related Dystrophies; Waardenburg's
Syndrome; Fasting Hypoglycemia; X-Linked Congenital Stationary Night Blindness; Malignant Hyperthermia Susceptibility; 48,
Xxxx; Tremor; Aneurysm; Chronic Pain; Thalassemia; Leukemia, Chronic Lymphocytic; Netherton Syndrome; Sjogren Syndrome; Perlman
Syndrome; Rothmund-Thomson Syndrome, Type 2; Clostridium Difficile Colitis; Covid-19; Pulmonary Fibrosis, Idiopathic; Muscular
Dystrophy, Limb-Girdle, Autosomal Recessive 4; Poikiloderma with Neutropenia; Myopathy, Proximal, with Ophthalmoplegia;
Glucocorticoid Resistance, Generalized; Alstrom Syndrome; Intellectual Developmental Disorder, X-Linked 21; Cognitive Function 1,
Social; Medulloblastoma; Danon Disease; Limb Ischemia; Dementia; Conjunctivitis; Neutropenia; Ehlers-Danlos Syndrome; Polyneuropathy;
Phaeohyphomycosis; Vaginal Discharge; Hyperglycemia; Bullous Keratopathy; Keratopathy; Acute Disseminated Encephalomyelitis;
Thyroiditis; Soft Tissue Sarcoma; Pathologic Nystagmus; Pachygyria; Depression; Overgrowth Syndrome; Optic Atrophy 1; Muscular
Dystrophy-Dystroglycanopathy, Type a, 1; Hypertriglyceridemia 1; Schwartz-Jampel Syndrome, Type 1; Moyamoya Disease 1; Left Bundle
Branch Hemiblock; Usher Syndrome; Thrombotic Thrombocytopenia Purpura; Neural Tube Defects; Fibrodysplasia Ossificans Progressiva;
Gastroesophageal Reflux; Meniere Disease; Diamond-Blackfan Anemia 2; Night Blindness, Congenital Stationary, Type la; Retinoschisis 1,
X-Linked, Juvenile; Retinitis Pigmentosa-Deafness Syndrome; Type 1 Diabetes Mellitus; Acute Kidney Failure; Leukemia;
Epidermolysis Bullosa Dystrophica; Pneumonia; Osteochondrodysplasia; Purpura; Interstitial Lung Disease; Heart Septal Defect;
Compartment Syndrome; Acne; Adenoma; Ileus; Chronic Kidney Disease; Mitochondrial Encephalomyopathy; Measles; Ltbp4-Related Cutis
Laxa; Spinocerebellar Degeneration; Nonsyndromic Hearing Loss; Primary Adrenal Insufficiency; Leukemia, Chronic Lymphocytic 2;
Pseudoachondroplasia; Blood Group--Kell System; Cardiomyopathy, Familial Hypertrophic, 2; Central Core Disease of Muscle;
Rhabdomyosarcoma 2; Human Cytomegalovirus Infection; Frontometaphyseal Dysplasia; Cholelithiasis; Congenital Hypothyroidism;
Hypophosphatemia; Juvenile Glaucoma; Epicanthus; Exudative Vitreoretinopathy 1; Charcot-Marie-Tooth Disease, Axonal, Type 2e;
Progressive Familial Heart Block, Type Ib; Chorea, Childhood-Onset, with Psychomotor Retardation; Myopathy, Distal, with Rimmed
Vacuoles; Deafness, X-Linked 1; Frontometaphyseal Dysplasia 1; Budd-Chiari Syndrome; Myocardial Infarction; Ectodermal Dysplasia-
Syndactyly Syndrome 2; Alpha/beta T-Cell Lymphopenia with Gamma/delta T-Cell Expansion, Severe Cytomegalovirus Infection, and
Autoimmunity; Cardiomyopathy, Dilated, 1x; Apnea, Central Sleep; Adrenal Hyperplasia, Congenital, Due to 21-Hydroxylase Deficiency;
Thyroid Carcinoma, Familial Medullary; Intellectual Developmental Disorder, X-Linked 29; Orofaciodigital Syndrome Viii; Myopathy,
Centronuclear, X-Linked; Progressive Relapsing Multiple Sclerosis; Duane Retraction Syndrome; Choreatic Disease; Hemopericardium;
Cardiac Tamponade; Right Bundle Branch Block; Candidiasis; Pseudohermaphroditism; Laryngitis; Multiple Endocrine Neoplasia;
Tricuspid Valve Insufficiency; Mouth Disease; Superior Mesenteric Artery Syndrome; Histiocytosis; Pericardial Effusion; Clubfoot;
Testicular Disease; Thyroid Gland Medullary Carcinoma; Cerebrovascular Disease; Mitochondrial Metabolism Disease; Hypertrichosis;
Acute Myocardial Infarction; Skin Melanoma; Dyskinesia of Esophagus; Dysautonomia; Congenital Hydrocephalus; Athetosis; Progeroid
Syndrome; Muscular Lipidosis; Laminin Subunit Alpha 2-Related Congenital Muscular Dystrophy; Cerebrofacial Arteriovenous Metameric
Syndrome; Isolated Duane Retraction Syndrome; Thyroid Carcinoma; Arteries, Anomalies of; Lipoid Congenital Adrenal Hyperplasia;
Multiple System Atrophy 1; Chediak-Higashi Syndrome; Pneumothorax, Primary Spontaneous; Lymphoproliferative Syndrome; Pre-Eclampsia;
Myelodysplastic Syndrome; Familial Adenomatous Polyposis; Myopathy, Lactic Acidosis, and Sideroblastic Anemia; Hashimoto Thyroiditis;
Muscular Dystrophy-Dystroglycanopathy, Type C, 4; 46, xy Sex Reversal 2; Cardiomyopathy, Dilated, 1 g; Sickle Cell Anemia; Muscular
Dystrophy, Limb-Girdle, Autosomal Recessive 10; Ataxia and Polyneuropathy, Adult-Onset; Leukemia, Acute Myeloid; Macular Degeneration,
Age-Related, 1; Muscular Dystrophy, Limb-Girdle, Autosomal Dominant 3; Aspergillosis; Muscular Dystrophy, Limb-Girdle, Autosomal
Dominant 1; Polydactyly; Methylmalonic Acidemia and Homocysteinemia, Cblx Type; Rett Syndrome; Adenomyosis; Myotonic Dystrophy 2;
Intellectual Developmental Disorder, X-Linked 41; Incontinentia Pigmenti; Ornithine Transcarbamylase Deficiency, Hyperammonemia Due
to; Coffin-Lowry Syndrome; Mucopolysaccharidosis, Type Ii; Paralytic Poliomyelitis; Primary Progressive Multiple Sclerosis; Neuronal
Ceroid Lipofuscinosis; Mood Disorder; Postpoliomyelitis Syndrome; Avoidant Personality Disorder; Personality Disorder; Dysentery;
Guillain-Barre Syndrome; Basilar Artery Occlusion; Squamous Cell Papilloma; Megacolon; Hyperuricemia; Lactic Acidosis;
Hermaphroditism; Toxic Megacolon; T Cell Deficiency; Goiter; Retinal Ischemia; Inguinal Hernia; Thrombosis; Craniosynostosis with
Fibular Aplasia; Retinal Vascular Disease; Papilloma; Sensory Peripheral Neuropathy; Lipoprotein Quantitative Trait Locus;
Fibromyalgia; Overnutrition; Liver Disease; Peptic Ulcer Disease; Interstitial Keratitis; Sideroblastic Anemia; Juvenile
Retinoschisis; Limb-Girdle Muscular Dystrophy Type 1a; Pattern Dystrophy; Pellucid Marginal Degeneration; X-Linked Congenital
Retinoschisis; 46, Xy Disorders of Sexual Development; Limb-Girdle Muscular Dystrophy Type 1b; Limb-Girdle Muscular Dystrophy Type 1c;
Genetic Skeletal Muscle Disease; Ventilator-Induced Diaphragmatic Dysfunction; Mesial Temporal Lobe Epilepsy with Hippocampal
Sclerosis; Acute Adrenal Insufficiency; Atherosclerosis Susceptibility; Noonan Syndrome 1; Ovarian Cancer; Dowling-Degos Disease 1;
Lymphatic Malformation 5; Antigen Defined by Monoclonal Antibody Aj9; Myopathy, Congenital, with Fiber-Type Disproportion; Obesity-
Hypoventilation Syndrome; Ocular Motor Apraxia; Mitochondrial Complex I Deficiency, Nuclear Type 1; Muscular Dystrophy, Limb-Girdle,
Autosomal Recessive 8; Respiratory Distress Syndrome in Premature Infants; Bacterial Infectious Disease; Actn3 Deficiency; Tetralogy
of Fallot; Sarcoidosis 1; Parkinson Disease, Late-Onset; Progressive External Ophthalmoplegia with Mitochondrial Dna Deletions,
Autosomal Dominant 1; Myopathy, Tubular Aggregate, 1; Chromosome 3q29 Deletion Syndrome; Progressive External Ophthalmoplegia with
Mitochondrial Dna Deletions, Autosomal Dominant 4; Salih Myopathy; Major Depressive Disorder; Methylmalonic Aciduria and
Homocystinuria, Cblc Type; Retinitis Pigmentosa 3; Progressive External Ophthalmoplegia with Mitochondrial Dna Deletions, Autosomal
Dominant 2; Nemaline Myopathy 1; Glut1 Deficiency Syndrome 2; Nasopharyngeal Carcinoma; Dengue Virus; Peripartum Cardiomyopathy;
Impaired Intellectual Development and Distinctive Facial Features with or Without Cardiac Defects; Hemophilia B; Malaria; Hamamy
Syndrome; Night Blindness, Congenital Stationary, Type 1e; Mucopolysaccharidosis-Plus Syndrome; Mental Retardation, Autosomal
Dominant 7; Beta-Thalassemia; Breasts and/or Nipples, Aplasia or Hypoplasia of, 1; Kearns-Sayre Syndrome; Retinitis Pigmentosa 11;
Stroke, Ischemic; Menkes Disease; Nemaline Myopathy 3; Linear Skin Defects with Multiple Congenital Anomalies 1; Third-Degree
Atrioventricular Block; Tracheomalacia; Ifap Syndrome 2; Severe Pre-Eclampsia; Adenocarcinoma; Squamous Cell Carcinoma; Childhood
Absence Epilepsy; Dysthymic Disorder; Cholera; Anal Squamous Cell Carcinoma; Adenosine Deaminase Deficiency; Posterior Myocardial
Infarction; Lymphopenia; Thrombocytopenia; Graves' Disease; Chronic Progressive External Ophthalmoplegia; Newborn Respiratory
Distress Syndrome; Primary Biliary Cholangitis; Olivopontocerebellar Atrophy; Gastroenteritis; Optic Nerve Disease; Enthesopathy;
Focal Epilepsy; Mental Depression; Fibrosarcoma; Placental Insufficiency; Cystic Lymphangioma; Egg Allergy; Rhinitis; Intracranial
Embolism; Neurilemmoma; Mesenchymal Cell Neoplasm; Middle East Respiratory Syndrome; Eclampsia; Autosomal Dominant Non- Syndromic
Intellectual Disability 5; Epidermolysis Bullosa Simplex with Muscular Dystrophy; Peripheral Vascular Disease; Angina Pectoris;
Prion Disease; Neuroblastoma; Viral Infectious Disease; Cholangitis; in Situ Carcinoma; Collagen Disease; Polyhydramnios; Atp7a-
Related Copper Transport Disorders; Dyrk1a Syndrome; Grin1-Related Neurodevelopmental Disorder; Cap Myopathy; Splenomegaly; Gigantism;
Iqsec2; Pseudo-Turner Syndrome; Neuropathy; Chilaiditi Syndrome; Childhood-Onset Nemaline Myopathy; Broken Heart Syndrome; Syngap1-
Related Intellectual Disability; Homologous Wasting Disease; Necrotizing Autoimmune Myopathy; Methylmalonic Acidemia with
Homocystinuria; Encephalitis; Intermediate Congenital Nemaline Myopathy; Paroxysmal Exertion-Induced Dyskinesia; Pediatric Multiple
Sclerosis; Hypereosinophilic Syndrome; Specific Language Disorder; Periodic Paralysis; Univentricular Heart; Qualitative or
Quantitative Defects of Beta-Sarcoglycan; Acute Generalized Exanthematous Pustulosis; Disorder of Copper Metabolism; Headache;
Tracheobronchomalacia; Seizure Disorder; Metabolic Myopathy; Inclusion Body Myopathy with Early-Onset Paget Disease with or Without
Frontotemporal Dementia 1; Prostate Cancer; Volvulus of Midgut; Acyl-Coa Dehydrogenase, Very Long-Chain, Deficiency of; Arachnoid
Cysts, Intracranial; Bladder Cancer; Candidiasis, Familial, 1; Cone-Rod Dystrophy 2; Jalili Syndrome; Schopf-Schulz-Passarge Syndrome;
Carpal Tunnel Syndrome; Clubfoot, Congenital, with or Without Deficiency of Long Bones and/or Mirror-Image Polydactyly;
Retinoblastoma; Kabuki Syndrome 1; Migraine with or Without Aura 1; Leprosy 3; Myosclerosis, Autosomal Recessive; Nemaline Myopathy 2;
Sialuria; Fryns Syndrome; Dihydrolipoamide Dehydrogenase Deficiency; D-Bifunctional Protein Deficiency; Periodontitis, Chronic;
Exanthem; Peripheral Artery Disease; Pontocerebellar Hypoplasia; 3-Methylglutaconic Aciduria; Mitochondrial Dna Depletion Syndrome;
Neurofibromatosis, Type I; Hemophagocytic Lymphohistiocytosis; Chromosome 16p11.2 Deletion Syndrome; Syndromic X-Linked Intellectual
Disability Snyder Type; Intestinal Pseudo-Obstruction; Leber Plus Disease; Non-Syndromic X-Linked Intellectual Disability 2;
Strabismus; Exostoses, Multiple, Type I; Gnathodiaphyseal Dysplasia; Nondisjunction; Hypercholesterolemia, Familial, 1; Cholestasis,
Intrahepatic, of Pregnancy, 1; Leiomyoma, Uterine; Frontotemporal Dementia and/or Amyotrophic Lateral Sclerosis 1; Facial Spasm;
Kleefstra Syndrome 1; Muscular Dystrophy, Limb-Girdle, Autosomal Recessive 12; Cavitary Optic Disc Anomalies; Wilson Disease; Night
Blindness, Congenital Stationary, Type 2a; Thiamine Metabolism Dysfunction Syndrome 2; Nemaline Myopathy 4; Meningioma, Radiation-
Induced; Accelerated Tumor Formation; Arthrogryposis, Mental Retardation, and Seizures; Muscular Dystrophy-Dystroglycanopathy, Type
C, 7; Tremor, Hereditary Essential, 5; Developmental and Epileptic Encephalopathy 75; Neuronal Ceroid-Lipofuscinoses; Mandibular
Hypoplasia, Deafness, Progeroid Features, and Lipodystrophy Syndrome; Encephalopathy, Progressive, Early-Onset, with Brain Edema and/
or Leukoencephalopathy, 1; Mycobacterium Tuberculosis 1; Hypophosphatemic Rickets, X-Linked Recessive; Fragile X Tremor/ataxia
Syndrome; Pigmentary Disorder, Reticulate, with Systemic Manifestations, X-Linked; Nance-Horan Syndrome; Chondrodysplasia Punctata 2,
X-Linked Dominant; Choroideremia; Paget Disease of Bone 2, Early-Onset; Aromatic L-Amino Acid Decarboxylase Deficiency; Cervical
Cancer; Muscular Dystrophy-Dystroglycanopathy, Type a, 7; Cardiomyopathy, Dilated, Iii; Mitochondrial Dna Depletion Syndrome 13; 3-
Methylglutaconic Aciduria, Type Vii; Muscular Dystrophy-Dystroglycanopathy, Type B, 2; Premature Ovarian Failure 7; Muscular
Dystrophy-Dystroglycanopathy, Type C, 2; Frontotemporal Dementia and/or Amyotrophic Lateral Sclerosis 6; Muscular Dystrophy-
Dystroglycanopathy, Type a, 2; Canavan Disease; Thymoma, Familial; Citrullinemia, Type Ii, Adult-Onset; Cardiomyopathy, Familial
Restrictive, 3; Lesch-Nyhan Syndrome; Severe Combined Immunodeficiency, X-Linked; Intellectual Developmental Disorder, X-Linked,
Syndromic, Snyder-Robinson Type; Pyruvate Dehydrogenase E1-Alpha Deficiency; Chondrosarcoma; Perrault Syndrome 1; Cholestasis,
Progressive Familial Intrahepatic, 2; Fryns Microphthalmia Syndrome; Cardiomyopathy, Dilated, 1d; Macular Degeneration, X-Linked
Atrophic; Developmental and Epileptic Encephalopathy 1; Renpenning Syndrome 1; Prostatic Hyperplasia, Benign; X Inactivation,
Familial Skewed, 1; Adrenoleukodystrophy; Pettigrew Syndrome; Intellectual Developmental Disorder, X-Linked, Syndromic, Lujan-Fryns
Type; Ubiquitin-Activating Enzyme, Y-Linked; Tooth Agenesis; Coenzyme Q10 Deficiency Disease; Perrault Syndrome; Tongue Squamous
Cell Carcinoma; Oral Squamous Cell Carcinoma; Syndromic X-Linked Intellectual Disability 14; Progressive Familial Intrahepatic
Cholestasis; Gynecomastia; Leiomyoma; Color Blindness; Prostatic Adenoma; Paraplegia; Demyelinating Polyneuropathy; Essential Tremor;
Chronic Inflammatory Demyelinating Polyradiculoneuropathy; Detrusor Sphincter Dyssynergia; Familial Hypercholesterolemia;
Angioedema; Cerebral Degeneration; Gaucher's Disease; Amelogenesis Imperfecta; Thymoma; Olfactory Neuroblastoma; Transient
Cerebral Ischemia; Aortic Aneurysm; Junctional Epidermolysis Bullosa; Malignant Astrocytoma; Complex Regional Pain Syndrome;
Neuromuscular Junction Disease; Rectosigmoid Cancer; Macular Retinal Edema; Systemic Scleroderma; Skull Base Meningioma; Corneal
Dystrophy; Kidney Cancer; Prostatic Hypertrophy; Adult Respiratory Distress Syndrome; Pulmonary Edema; Hemiplegia; Infant
Gynecomastia; Congenital Muscular Dystrophy-Dystroglycanopathy A7; Congenital Muscular Dystrophy-Dystroglycanopathy Type A2; Non-
Alcoholic Steatohepatitis; Cystinosis; Osteopetrosis; Achromatopsia; Allergic Disease; Atrial Fibrillation; Lung Cancer; Panniculitis;
Urticaria; Dental Caries; Cholestasis; Gastritis; Axonal Neuropathy; Clear Cell Meningioma; Acquired Immunodeficiency Syndrome;
Retinal Degeneration; Vasculitis; Mesenchymal Chondrosarcoma; Biotin-Thiamine-Responsive Basal Ganglia Disease; Free Sialic Acid
Storage Disorders; Giant Axonal Neuropathy; Chronic Fatigue Syndrome; Amyloidosis; Periodontitis; Stenotrophomonas Maltophilia
Infection; Encephalopathy; Congenital Nystagmus; Hereditary Neuropathies; Coccygodynia; Glioma; Anca-Associated Vasculitis; Auriculo-
Condylar Syndrome; Chromosome Xp Deletion; Corticobasal Degeneration; Mechanical Strabismus; Trichorhinophalangeal Syndrome;
Adrenomyeloneuropathy; Exencephaly; Hansen's Disease; Precocious Puberty; Madelung Deformity; Ocular Albinism, X-Linked; Rrm2b
Mitochondrial Dna Maintenance Defects; Diabetic Neuropathy; Glial Tumor; Familial Intrahepatic Cholestasis; Spastic Paraplegia-Paget
Disease of Bone Syndrome; Rigid Spine Muscular Dystrophy; Traumatic Brain Injury; Laminin Subunit Alpha 2-Related Muscular Dystrophy;
Ring Chromosome; Homozygous Familial Hypercholesterolemia; Cerebral Aneurysms; Anoxia; Cerebral Hypoxia; Color Vision Deficiency;
Laminopathy; Familial Isolated Restrictive Cardiomyopathy; Polyploidy; Argyria; or Non-Syndromic Pontocerebellar Hypoplasia

### EXAMPLES

The following examples are included for illustrative purposes only and are not intended to limit the scope of the invention.

### Example 1: Indel Activity of CasPhi.32 Effector Protein (SEQ ID NO: 1)

Combinations of the effector protein (as set forth in SEQ ID NO: 1) and guide nucleic acids (TABLE 11) target various exons (loci) of the DMD gene, as represented in TABLE 9, were tested for their ability to produce indels in HEK293T cells. Sequences of targeted exons are as set forth in TABLE 9.3. Some indels are predicted to result in exon skipping.

Briefly, 300 ng of plasmids expressing the effector protein (as set forth in SEQ ID NO: 1) and transcribing targeting gRNA were delivered by lipofection to HEK293T cells in 96 well plates. TransIT-293 reagent was diluted with warmed up OPTIMEM and mixed with the plasmid DNA at the ratio of 2:1 lipid:DNA. Lipid:DNA mixture were incubated for 10 minutes at room temperature before adding 20 μL of the lipid:DNA optimem mixture to each well. Cells were incubated for 3 days before being lysed and subjected to PCR amplification. Each composition was assayed in two replicate batches. Indels were detected by next generation sequencing of PCR amplicons at the targeted loci and indel percentage was calculated as the fraction of sequencing reads containing insertions or deletions relative to the unedited DMD gene sequence, and are provided in TABLE 12.

TABLE 11
CASPHI.32 EFFECTOR PROTEIN (SEQ ID NO: 1) AND GUIDE NUCLEIC
ACID Combinations

| | | Repeat | Spacer | | |
|---|---|---|---|---|---|
| | | Sequence | Sequence | crRNA | Targeted |
| Name | PAM | SEQ ID NO: | SEQ ID NO: | SEQ ID NO: | Exon |
| T01_CasPhi32 | GTTA | 739 | 28 | 748 | 44 |
| T02_CasPhi32 | GTTT | 739 | 29 | 749 | 44 |
| T03_CasPhi32 | GTTA | 739 | 30 | 750 | 44 |
| T04_CasPhi32 | GTTT | 739 | 31 | 751 | 44 |
| T05_CasPhi32 | GTTC | 739 | 32 | 752 | 44 |
| T06_CasPhi32 | GTTA | 739 | 33 | 753 | 44 |
| T07_CasPhi32 | GTTG | 739 | 34 | 754 | 44 |
| T08_CasPhi32 | GTTT | 739 | 35 | 755 | 44 |
| T09_CasPhi32 | GTTG | 739 | 36 | 756 | 44 |
| T10_CasPhi32 | GTTT | 739 | 37 | 757 | 45 |
| T11_CasPhi32 | GTTC | 739 | 38 | 758 | 45 |
| T12_CasPhi32 | GTTT | 739 | 39 | 759 | 45 |
| T13_CasPhi32 | GTTG | 739 | 40 | 760 | 45 |
| T14_CasPhi32 | GTTT | 739 | 41 | 761 | 45 |
| T15_CasPhi32 | GTTG | 739 | 42 | 762 | 45 |
| T16_CasPhi32 | GTTA | 739 | 43 | 763 | 45 |
| T17_CasPhi32 | GTTT | 739 | 44 | 764 | 45 |
| T18_CasPhi32 | GTTG | 739 | 45 | 765 | 45 |
| T19_CasPhi32 | GTTC | 739 | 46 | 766 | 45 |
| T20_CasPhi32 | GTTA | 739 | 47 | 767 | 50 |
| T21_CasPhi32 | GTTA | 739 | 48 | 768 | 50 |
| T22_CasPhi32 | GTTA | 739 | 49 | 769 | 50 |
| T23_CasPhi32 | GTTC | 739 | 50 | 770 | 50 |
| T24_CasPhi32 | GTTA | 739 | 51 | 771 | 50 |
| T25_CasPhi32 | GTTC | 739 | 52 | 772 | 50 |
| T26_CasPhi32 | GTTT | 739 | 53 | 773 | 50 |
| T27_CasPhi32 | GTTT | 739 | 54 | 774 | 50 |
| T28_CasPhi32 | GTTG | 739 | 55 | 775 | 50 |
| T29_CasPhi32 | GTTA | 739 | 56 | 776 | 50 |
| T30_CasPhi32 | GTTT | 739 | 57 | 777 | 51 |
| T31_CasPhi32 | GTTA | 739 | 58 | 778 | 51 |
| T32_CasPhi32 | GTTA | 739 | 59 | 779 | 51 |
| T33_CasPhi32 | GTTT | 739 | 60 | 780 | 51 |
| T34_CasPhi32 | GTTT | 739 | 61 | 781 | 51 |
| T35_CasPhi32 | GTTT | 739 | 62 | 782 | 51 |
| T36_CasPhi32 | GTTT | 739 | 63 | 783 | 51 |
| T37_CasPhi32 | GTTG | 739 | 64 | 784 | 51 |
| T38_CasPhi32 | GTTC | 739 | 65 | 785 | 51 |
| T39_CasPhi32 | GTTG | 739 | 66 | 786 | 51 |
| T40_CasPhi32 | GTTT | 739 | 67 | 787 | 51 |
| T41_CasPhi32 | GTTC | 739 | 68 | 788 | 53 |
| T42_CasPhi32 | GTTG | 739 | 69 | 789 | 53 |
| T43_CasPhi32 | GTTG | 739 | 70 | 790 | 53 |
| T44_CasPhi32 | GTTT | 739 | 71 | 791 | 53 |
| T45_CasPhi32 | GTTA | 739 | 72 | 792 | 53 |
| T46_CasPhi32 | GTTA | 739 | 73 | 793 | 53 |
| T47_CasPhi32 | GTTG | 739 | 74 | 794 | 53 |
| T48_CasPhi32 | GTTA | 739 | 75 | 795 | 53 |
| T49_CasPhi32 | GTTA | 739 | 76 | 796 | 53 |
| T50_CasPhi32 | GTTC | 739 | 77 | 797 | 53 |
| T51_CasPhi32 | GTTA | 739 | 78 | 798 | 53 |
| T52_CasPhi32 | GTTA | 739 | 79 | 799 | 53 |
| T53_CasPhi32 | GTTA | 739 | 80 | 800 | 53 |
| T54_CasPhi32 | GTTT | 739 | 81 | 801 | 53 |
| T55_CasPhi32 | GTTT | 739 | 82 | 802 | 53 |

TABLE 12
Indel activity of Effector Protein (SEQ ID NO: 1) and
Guide Nucleic Acid Combinations set forth in TABLE 11

| | % INDEL | | % INDEL |
|---|---|---|---|
| Name | REP1 | % INDEL REP2 2 | AVERAGE |
| T01_CasPhi32 | 20.23846916 | 19.30862754 | 19.77354835 |
| T02_CasPhi32 | 0.015529369 | 0.021318404 | 0.018423887 |
| T03_CasPhi32 | 0.073060652 | 0.114070159 | 0.093565405 |
| T04_CasPhi32 | 0.623668844 | 0.578443281 | 0.601056062 |
| T05_CasPhi32 | 0.025062657 | 0.017263703 | 0.02116318 |
| T06_CasPhi32 | 0.001321336 | 0 | 0.000660668 |
| T07_CasPhi32 | 0.010896148 | 0.01679957 | 0.013847859 |
| T08_CasPhi32 | 0.001411014 | 0 | 0.000705507 |
| T09_CasPhi32 | 0.015678545 | 0.019173293 | 0.017425919 |
| T10_CasPhi32 | 0.002004028 | 0.003164423 | 0.002584226 |
| T11_CasPhi32 | 0.221117146 | 0.242306992 | 0.231712069 |
| T12_CasPhi32 | 0.028144833 | 0.024203078 | 0.026173956 |
| T13_CasPhi32 | 0.353075513 | 0.38499615 | 0.369035832 |
| T14_CasPhi32 | 0.070613658 | 0.092539409 | 0.081576534 |
| T15_CasPhi32 | 1.011045466 | 1.025548216 | 1.018296841 |
| T16_CasPhi32 | 0.597505881 | 0.738482939 | 0.66799441 |
| T17_CasPhi32 | 0.016460905 | 0.010401858 | 0.013431382 |
| T18_CasPhi32 | 0.033347676 | 0.048708712 | 0.041028194 |
| T19_CasPhi32 | 0.123840975 | 0.183951477 | 0.153896226 |
| T20_CasPhi32 | 0.030069022 | 0.050638455 | 0.040353739 |
| T21_CasPhi32 | 0.0658736 | 0.141764893 | 0.103819247 |
| T22_CasPhi32 | 0 | 0.003136222 | 0.001568111 |
| T23_CasPhi32 | 0.002899139 | 0.001386001 | 0.00214257 |
| T24_CasPhi32 | 0.013295908 | 0.012136291 | 0.012716099 |
| T25_CasPhi32 | 0.054448021 | 0.08662789 | 0.070537956 |
| T26_CasPhi32 | 0.008306941 | 0.007706758 | 0.00800685 |
| T27_CasPhi32 | 0.074932204 | 0.190795825 | 0.132864015 |
| T28_CasPhi32 | 0.216237663 | 0.202494961 | 0.209366312 |
| T29_CasPhi32 | 0.009013649 | 0.011105627 | 0.010059638 |
| T30_CasPhi32 | 0.01022988 | 0.016899028 | 0.013564454 |
| T31_CasPhi32 | 0.001260327 | 0.000621577 | 0.000940952 |
| T32_CasPhi32 | 0 | 0.001328551 | 0.000664275 |
| T33_CasPhi32 | 0.121015532 | 0.104336161 | 0.112675847 |
| T34_CasPhi32 | 0.018999296 | 0.006905998 | 0.012952647 |
| T35_CasPhi32 | 0.587456753 | 0.672856311 | 0.630156532 |
| T36_CasPhi32 | 0.073020918 | 0.008192357 | 0.040606637 |
| T37_CasPhi32 | 0.130643528 | 0.183844651 | 0.15724409 |
| T38_CasPhi32 | 0.277694707 | 0.378249129 | 0.327971918 |
| T39_CasPhi32 | 0.014505691 | 0.017499794 | 0.016002742 |
| T40_CasPhi32 | 0.021236833 | 0.018098378 | 0.019667605 |
| T41_CasPhi32 | 5.060466568 | 4.60575076 | 4.833108664 |
| T42_CasPhi32 | 2.521905369 | 2.840688774 | 2.681297071 |
| T43_CasPhi32 | 0.013254201 | 0.026622296 | 0.019938249 |
| T44_CasPhi32 | 0.03042185 | 0.030263817 | 0.030342833 |
| T45_CasPhi32 | 0.113670484 | 0.116834403 | 0.115252443 |
| T46_CasPhi32 | 0.017335529 | 0.024589989 | 0.020962759 |
| T47_CasPhi32 | 0.021419392 | 0.033736122 | 0.027577757 |
| T48_CasPhi32 | 0 | 0.029151042 | 0.014575521 |
| T49_CasPhi32 | 0.018055756 | 0.006806502 | 0.012431129 |
| T50_CasPhi32 | 0.019211066 | 0.013170498 | 0.016190782 |
| T51_CasPhi32 | 0.018894152 | 0.009048487 | 0.01397132 |
| T52_CasPhi32 | 0.13066077 | 0.139523085 | 0.135091927 |
| T53_CasPhi32 | 0.014060149 | 0.012559658 | 0.013309904 |
| T54_CasPhi32 | 0.002465331 | 0.003726106 | 0.003095719 |
| T55_CasPhi32 | 0.005324176 | 0.00126754 | 0.003295858 |

### Example 2: Indel Activity of CasPhi.32 Effector Protein (SEQ ID NO: 1)

Combinations of the CasPhi.32 effector protein (as set forth in SEQ ID NO: 1) and guide nucleic acids as set forth in TABLE 13 targeting the DMD gene were tested as described in Example 1 for their ability to produce indels in the DMD gene.

Indels were detected and calculated as set forth in Example 1, and indel activity of assayed combinations are provided in TABLE 14. Guide nucleic acids were designed for the CasPhi.32 effector protein to recognize a PAM of GTTN, wherein N is A, G, C, or T.

TABLE 13
CasPhi.32 Effector Protein (SEQ ID NO:
1) and Guide Nucleic Acid Combinations

| | Repeat Sequence | Spacer Sequence | crRNA SEQ |
|---|---|---|---|
| Name | SEQ ID NO: | SEQ ID NO: | ID NO: |
| T56_CasPhi32 | 739 | 83 | 803 |
| T57_CasPhi32 | 739 | 84 | 804 |
| T58_CasPhi32 | 739 | 85 | 805 |
| T59_CasPhi32 | 739 | 86 | 806 |
| T60_CasPhi32 | 739 | 87 | 807 |
| T61_CasPhi32 | 739 | 88 | 808 |
| T62_CasPhi32 | 739 | 89 | 809 |
| T63_CasPhi32 | 739 | 90 | 810 |
| T64_CasPhi32 | 739 | 91 | 811 |
| T65_CasPhi32 | 739 | 92 | 812 |
| T66_CasPhi32 | 739 | 93 | 813 |
| T67_CasPhi32 | 739 | 94 | 814 |
| T68_CasPhi32 | 739 | 95 | 815 |
| T69_CasPhi32 | 739 | 96 | 816 |
| T70_CasPhi32 | 739 | 97 | 817 |
| T71_CasPhi32 | 739 | 98 | 818 |
| T72_CasPhi32 | 739 | 99 | 819 |
| T73_CasPhi32 | 739 | 100 | 820 |
| T74_CasPhi32 | 739 | 101 | 821 |
| T75_CasPhi32 | 739 | 102 | 822 |
| T76_CasPhi32 | 739 | 103 | 823 |
| T77_CasPhi32 | 739 | 104 | 824 |
| T78_CasPhi32 | 739 | 105 | 825 |
| T79_CasPhi32 | 739 | 106 | 826 |
| T80_CasPhi32 | 739 | 107 | 827 |
| T81_CasPhi32 | 739 | 108 | 828 |
| T82_CasPhi32 | 739 | 109 | 829 |
| T83_CasPhi32 | 739 | 110 | 830 |
| T84_CasPhi32 | 739 | 111 | 831 |

TABLE 14
Indel activity of CasPhi.32 Effector Protein (SEQ
ID NO: 1) and Guide Nucleic Acid Combinations

| Name | % indel rep 1 | % indel rep 2 |
|---|---|---|
| T56_CasPhi32 | 0 | 0 |
| T57_CasPhi32 | 0 | 0 |
| T58_CasPhi32 | 0.797846 | 0.390734 |
| T59_CasPhi32 | 0.175747 | 0.132275 |
| T60_CasPhi32 | 0.250376 | 0.530244 |
| T61_CasPhi32 | 0.011172 | 0 |
| T62_CasPhi32 | 0.438614 | 0.286041 |
| T63_CasPhi32 | 0 | 0.044346 |
| T64_CasPhi32 | 0.676739 | 0.56338 |
| T65_CasPhi32 | 0.266302 | 0.065714 |
| T66_CasPhi32 | 14.7318 | 16.25719 |
| T67_CasPhi32 | 0 | 0.274691 |
| T68_CasPhi32 | 0.054825 | 0 |
| T69_CasPhi32 | 0 | 0 |
| T70_CasPhi32 | 0 | 0.062814 |
| T71_CasPhi32 | 0 | 0.024181 |
| T72_CasPhi32 | 11.485 | 10.01793 |
| T73_CasPhi32 | 0.04754 | 0 |
| T74_CasPhi32 | 0.003291 | 0.013661 |
| T75_CasPhi32 | 0 | 0 |
| T76_CasPhi32 | 0 | 0 |
| T77_CasPhi32 | 0 | 0 |
| T78_CasPhi32 | 0 | 0 |
| T79_CasPhi32 | 0.037979 | 0.065717 |
| T80_CasPhi32 | 0.022591 | 0.011996 |
| T81_CasPhi32 | 0.064921 | 0.023852 |
| T82_CasPhi32 | 0.010943 | 0 |
| T83_CasPhi32 | 0 | 0 |
| T84_CasPhi32 | 0 | 0 |

### Example 3: Indel Activity and Splicing Disruptions and Frameshifts Analysis

Indel activity of effector proteins (e.g., TABLE 1 or variants thereof) can be used to predict frameshift and splicing interruptions. Specifically, upon NGS sequencing, the location and number of indels (“reads”) can be used to predict exon-specific frameshifts, splicing interruptions, and other mutations.

Splicing interruption: Briefly, splicing interruptions can be predicted based on the location of the coding sequence overlaid on an amplicon, by counting the number of reads where there is an indel on the first 2 bases before or after the coding sequence. When indel activity reaches the edge of a coding sequence (i.e., the end or start of the coding sequence), indel counting for splice disruption analysis would not begin until after or before the end or start of the coding sequence, respectively.

Frameshifts: Frameshifts are predicted by counting all reads that are modified but not predicted for splicing interruption, and have a specific indel size.

A specific indel size depends on the frame shift and can be calculated by a modulo operation. A modulo operation is an action that given two positive integers, the operation returns the remainder after one integer is divided by another. Generally, a modulo operation can be represented by the formula (a mod n) where a is the dividend and n is the divisor. For example, the expression “5 mod 2” would evaluate to 1, because 5 divided by 2 has a quotient of 2 and a remainder of 1, while “9 mod 3” would evaluate to 0, because the division of 9 by 3 has a quotient of 3 and a remainder of 0; there is nothing to subtract from 9 after multiplying 3 times 3.

Here, frameshifts can be predicted by using the following formula:

$$x mod 3 = y , \qquad Equation 1$$

where x is the number of modified reads, 3 is divisor and the remainder y gives the frameshift (i.e., 2, 1 or 0).

Per equation 1, the number of modified reads is divided by 3 if the remainder is 2, then a 2 frameshift mutation is predicted; if the remainder is 1, then a 1 frameshift mutation is predict, and if the remainder is 0, then an inframe mutation is predict. Inframe mutations also include where there are 0 modified reasons.

Modified reads are changes that are done in the quantification window. The quantification window is a window defined based on the effector's splicing position. It is used by the tool to define a real modification vs NGS errors. If a modification is done within the window the read is counted as modified, otherwise it is considered unmodified. An example could be an amplicon with a poly T region far from predicted splicing site, those regions can often show deletions but are actually an NGS artifact.

Other Mutations: Other mutations can also be predicted based on the location and number of indels, or based on other factors.

Indel Patterns: Analysis of splicing disruptions and frameshift mutations is used to pattern mutations as a function of indel % range for each targeted exon. Hypothetical ranges for exon-specific indel cutting patterns can be seen in TABLE 15 below.

TABLE 15
Hypothetical Exon-Specific Indel Cutting Patterns

| | % indel, | % indel, | % indel, | % indel, | % indel, |
|---|---|---|---|---|---|
| Mutation | exon A | exon B | exon C | exon D | exon E |
| 2 frameshift | 0 to 2.5% | 0 to 5% | 0 to 10% | 0 to 15% | 0 to 20% or |
| | | | | | more |
| 1 frameshift | 2.5% or less | 5% to 15% | 10% to 20% | 15% to 25% | 20% to 25% |
| | to 3.5% | | | | or more |
| splice | 3.5% or less | 15% to 17% | 20% to 25% | 25% to 27% | 25% to 30% |
| disruption | to 15% | | | | or more |
| Other | n/a | 17% to 25% | n/a | n/a | n/a |

### Example 4: Indel Activity of Effector Protein (e.g., TABLE 1, or a Variant Thereof) in Cardiomyocytes: Lipofection, Viability and Expression of eGFP (Plasmid and mRNA Delivery) and Indel Activity in iPSC-Derived Cardiomyocytes

### Example 4.1: Lipofection. Viability and Expression of eGFP (Plasmid and mRNA Delivery) in iPSC-Derived Cardiomyocytes

Example 4.1.1: Lipofection of iPSC derived cardiomyocytes: Briefly, iPSC derived cardiomyocytes is purchased and cultured according to Takara Bio Europe AB, Cellartis® Cardiomyocytes User Manual. Cat. No. Y10075, pp. 1-6 (2018). Plasmid or mRNA encoding GFP are delivered by lipofection as described in ThermoFisher Scientific, Lipofectamine™ Stem Transfection Reagent. Pub. No. MAN0017080, pp. 1-2 (2017) and in TAN et al., “Non-viral vector based gene transfection with human induced pluripotent stem cells derived cardiomyocytes,” Sci. Reports, 9:14404 (2019) (modifying ThermoFischer Scientific, 2017 in terms of kit and lipid to DNA ratio). Results will demonstrate successful lipofection of iPSC derived cardiomyocytes.

Example 4.1.2: Cardiomyocytes GFP mRNA and plasmid expression after 48 h: GFP positivity of mRNA and plasmid delivered cardiomyocytes are measured 48 hours after lipofection by flow cytometry to establish the incidence of GFP expression. Mean fluorescence intensity (MFI) is measured 48 hours after lipofection by flow cytometry to establish the level of GFP expression. Results will demonstrate successful integration lipofection delivery of GFP in cardiomyocytes.

### Example 4.2: Indel Activity of Effector Protein in Cardiomyocytes Compared to a GFP Control

Plasmids expressing effector protein/guide nucleic acid combinations and eGFP targeting the DMD gene are delivered by lipofection to iPSC derived cardiomyocytes as set forth in Example 4.1.1. Effector protein (e.g., an effector protein set forth in TABLE 1, or a variant thereof) and guide nucleic acid combinations (e.g., combinations of one or more of: TABLE 4, TABLE 6, TABLE 7, and TABLE 8) are delivered on the same vector.

Single and Dual cutting is assessed by delivery of one or two guides, respectively. GFP expression and indel activity is assessed 72 hours post lipofection. Results indicate indel activity. Prediction of indels are made based on NGS data as described in Example A.3. Results will demonstrate that effector protein and guide nucleic acid combinations can be predicted to effect in-frame, +1 frameshift, +2 frameshift mutations, splicing disruption, and/or full sequence deletion/dual cutting. Splice disruptions mutations and 1+ frameshift mutations are predicted to be the most helpful for DMD gene editing.

### Example 5: Indel Activity of Effector Protein (e.g., TABLE 1, or a Variant Thereof) in Myoblasts: Lipofection, Viability and Expression of eGFP (Plasmid and mRNA Delivery) and Indel Activity in iPSC-Derived Myoblasts

### Example 5.1: Lipofection. Viability and Expression of eGFP (Plasmid and mRNA Delivery) in iPSC-Derived Myoblasts

Example 5.1.1: Lipofection of iPSC derived myoblasts: iPSC derived myoblasts are purchased and cultured according to Life Technologies Corporation. HSkM-S. Cat. No. A12555, pp. 1-2 (2010). Plasmid or mRNA encoding GFP are delivered by lipofection as described in ThermoFisher Scientific. Lipofectamine™ Stem Transfection Reagent. Pub. No. MAN0017080, pp. 1-2 (2017) and in TAN et al., “Non-viral vector based gene transfection with human induced pluripotent stem cells derived cardiomyocytes.” Sci. Reports. 9:14404 (2019) as described for cardiomyocytes above. Results will demonstrate successful lipofection of iPSC derived myoblasts.

Example 5.1.2: Myoblasts GFP mRNA and plasmid expression after 48 h: GFP positivity of mRNA and plasmid delivered myoblasts are measured 48 hours after lipofection by flow cytometry to establish the incidence of GFP expression. Mean fluorescence intensity (MFI) is measured 48 hours after lipofection by flow cytometry to establish the level of GFP expression. Results will demonstrate successful integration lipofection delivery of GFP in cardiomyocytes.

### Example 5.2: Indel Activity of Effector Protein in Myoblasts Compared to GFP Control

Single and Dual cutting is assessed by delivery of one or two guides, respectively, and effector protein (e.g., an effector protein set forth in TABLE 1, or a variant thereof) combinations (e.g., combinations of one or more of: TABLE 4. TABLE 6. TABLE 7, and TABLE 8). GFP expression and indel activity are assessed 72 hours post lipofection. Results will indicate indel activity of assayed effector proteins.

Prediction of mutations are made based on NGS data as described in Example 3. Results will demonstrate that effector protein and guide nucleic acid combinations can be predicted to effect in-frame and +1 frameshift mutations. Some activity will also be seen in two guide systems.

### Example 6: AAV Vectors for Gene Editing by a Single Cut

An AAV vector is constructed to contain a transgene between its ITRs, the transgene providing or encoding, in a 5′ to 3′ direction, a nucleotide sequence of a first promoter, a nucleotide sequence encoding a guide nucleic acid, a nucleotide sequence of a second promoter, a nucleotide sequence encoding an effector protein, an enhancer, and a poly A signal sequence as illustrated in FIG. 2 are packaged into an AAV vector. The effector protein has an amino acid sequence that has at least 80% identity to any one of sequences recited in TABLE 1, or a variant thereof. The guide nucleic acid comprises a spacer sequence that binds to a target sequence in a target nucleic acid and has at least 90% identity to a sequence recited in TABLE 4. As illustrated in FIG. 2, the effector protein can be expressed either ubiquitously or in a specific muscle based on the promotor the AAV vector is engineered to have. The AAV vector has a second promotor U6. WPRE enhancer and hGH Poly A signal sequence. The AAV vector is expressed with supporting plasmids to produce an adeno-associated virus (AAV).

### Example 7: AAV Vectors for Gene Editing by a Dual Cut

An AAV vector is constructed to contain a transgene between its ITRs, the transgene providing or encoding, in a 5′ to 3′ direction, a nucleotide sequence of a first promoter, a nucleotide sequence encoding a first guide nucleic acid, a nucleotide sequence of a second promoter, a nucleotide sequence encoding an effector protein, an enhancer, a poly A signal, a nucleotide sequence of a third promotor, and a nucleotide sequence encoding a second guide nucleic acid as illustrated in FIG. 3 are packaged into an AAV vector. The effector protein has an amino acid sequence that has at least 80% identity to any one of sequences recited in TABLE 1, or a variant thereof. The first guide nucleic acid and the second guide nucleic acid comprise different spacer sequences targeting them to different target sequences of DMD. The first guide nucleic acid and the second guide nucleic acid can independently comprises a spacer sequence that binds to a target sequence in a target nucleic acid and has at least 90% identity to a sequence recited in TABLE 4. In some examples, the first guide nucleic acid and the second guide nucleic acid are complementary to sequences 5′ and 3′ of a given exon, respectively. Therefore, the dual cut can remove the exon. As illustrated in FIG. 3, the effector protein can be expressed either ubiquitously or in a specific muscle based on the promotor the AAV vector is engineered to have. The AAV vector also has U6 first promotor, 7SK second promotor. WPRE enhancer, and hGH Poly A signal sequence. The AAV vector is expressed with supporting plasmids to produce an adeno-associated virus (AAV).

### Example 8: Gene Editing of iPSC-Derived Cardiomyocytes with AAV Vector Encoding Effector Protein and Guide Nucleic Acid

An AAV vector is constructed to contain a transgene between its ITRs according to any one of the constructs described in Example 6 and 7. The AAV vector is expressed with supporting plasmids to produce an adeno-associated virus (AAV), iPSC-derived cardiomyocytes are contacted with the AAV. After about 48-96 hours. DNA or RNA is isolated from the infected cells. An indel caused by the guide nucleic acid-effector protein complex is confirmed by sequencing and/or Q-PCR.

### Example 9: In Vivo Gene Editing in a Mammalian Model for Treating Muscular Dystrophy Mutation(s) by AAV

An AAV vector is constructed to contain a transgene between its ITRs according to any one of the constructs described in Example 6 and 7. The AAV vector is expressed with supporting plasmids to produce an adeno-associated virus (AAV). A mouse with muscular dystrophy is administered an effective dose of the AAV. About four weeks post administration, a sample muscle is extracted for analysis of dystrophin restoration. The sample muscle can be chosen based on the promotor used for expressing the effector protein. The analysis can be performed by any technique known to a skillful artisan, which includes but are not limited to immunohistochemistry, western blot analysis and deep-sequencing analysis. Similarly, rescue of pathological phenotypes can be determined by performing any technique known to a skillful artisan, which includes but are not limited to hematoxylin and cosin (H&E) staining. Masson's trichrome staining, grip-strength analysis, muscular electrophysiological analysis, and serum creatine kinase (CK).

### Example 10: Indel Activity of Cas Phi. 12 Effector Protein (SEQ ID NO: 2)

Combinations of the effector protein (SEQ ID NO: 2) and guide nucleic acids as set forth in TABLE 16 and target various exons (loci) of the DMD gene, as represented in TABLE 9 were tested for their ability to produce indels in HEK293T cells. Sequences of targeted exons are as set forth in TABLE 9.4. Some indels are predicted to result in exon skipping.

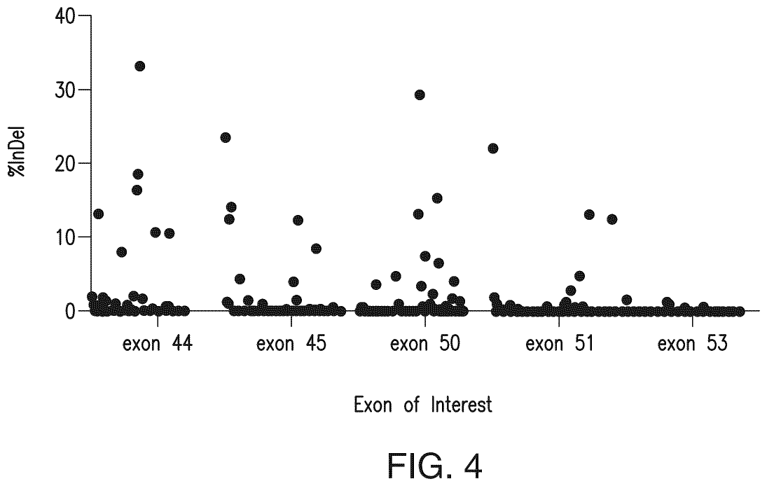

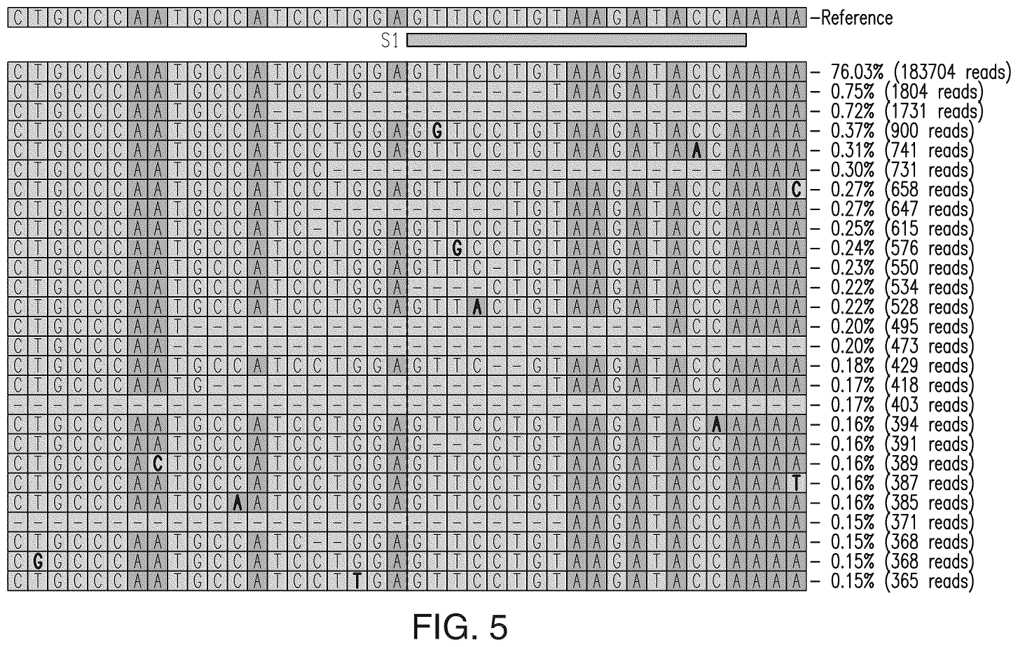

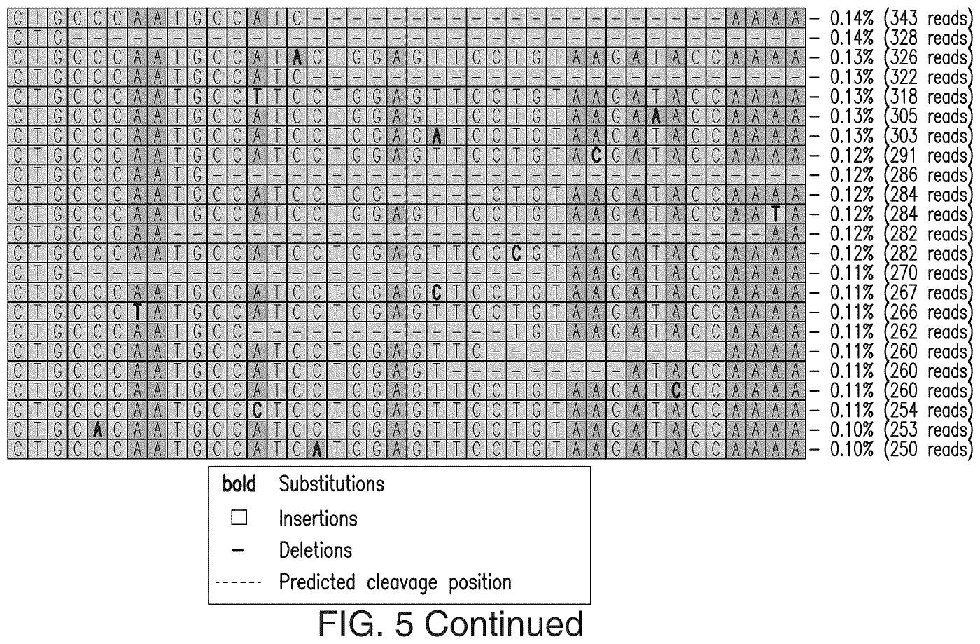

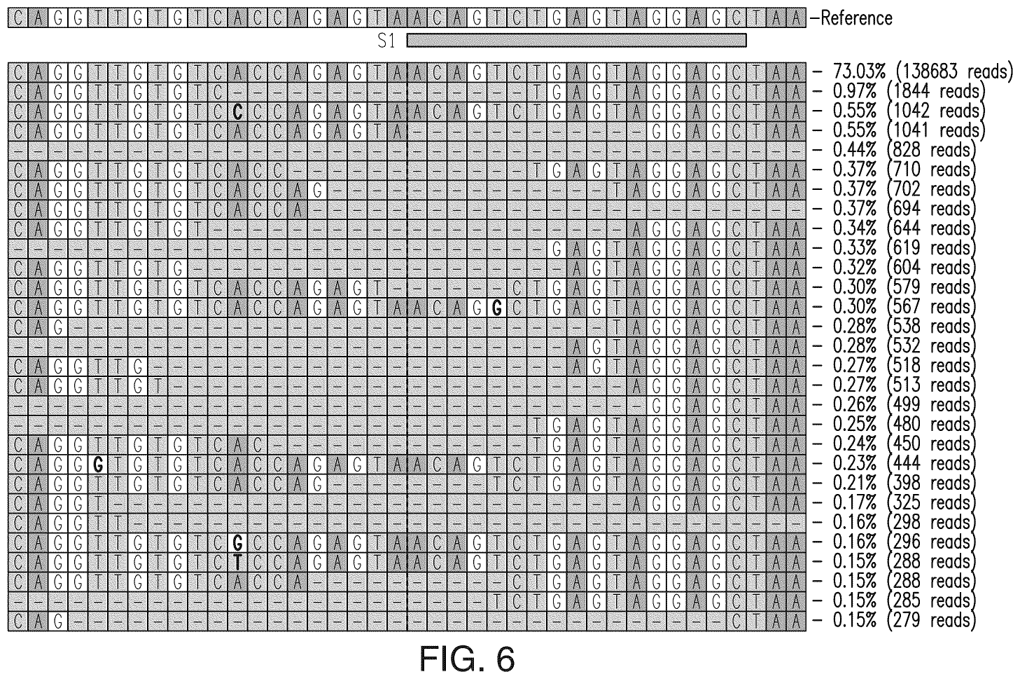

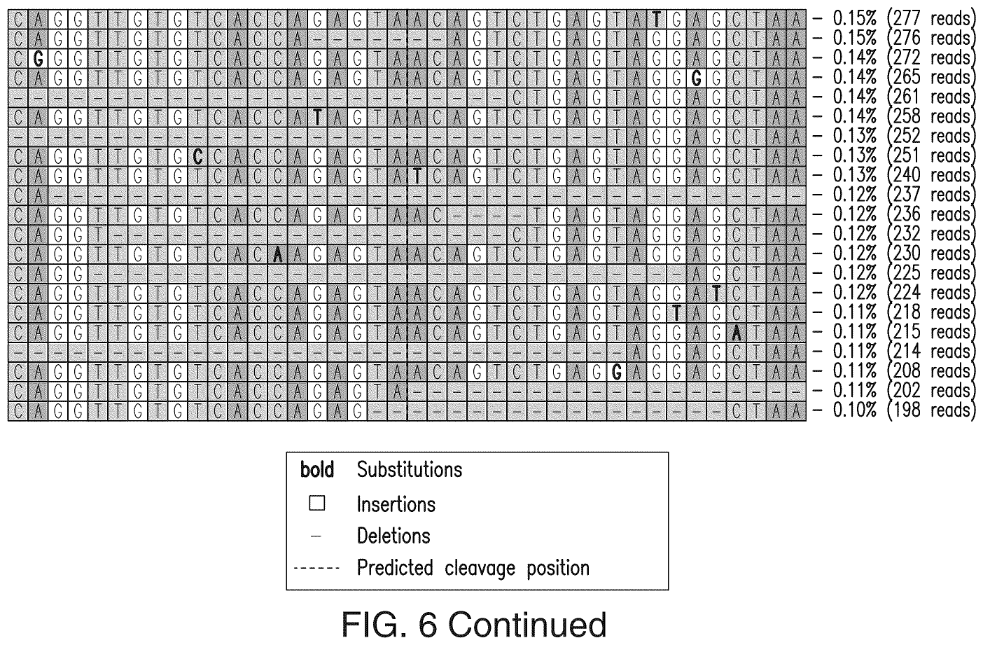

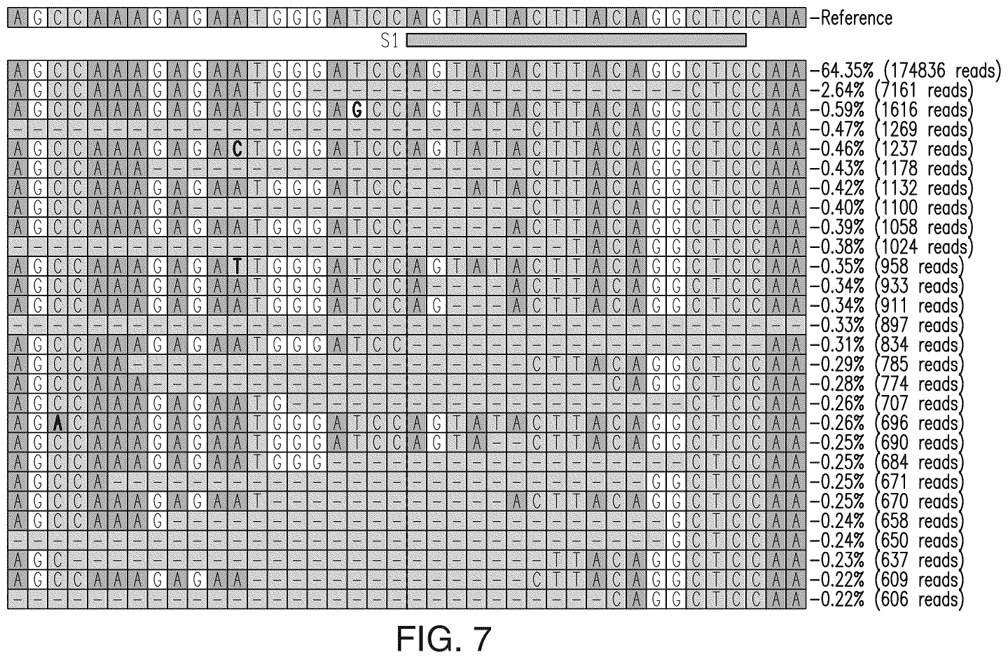

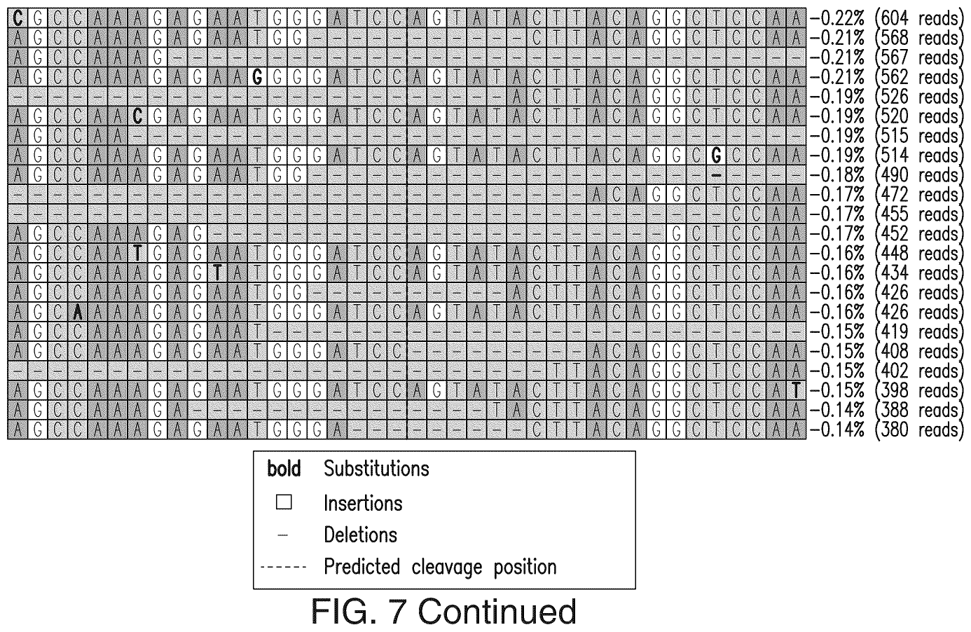

Briefly, 300 ng of plasmids expressing the effector protein (SEQ ID NO: 2) and transcribing targeting gRNA were delivered by lipofection to HEK293T cells in 96 well plates. TransIT-293 reagent was diluted with warmed up OPTIMEM and mixed with the plasmid DNA at the ratio of 2:1 lipid:DNA. Lipid:DNA mixture were incubated for 10 minutes at room temperature before adding 20 μL of the lipid:DNA optimem mixture to each well. Cells were incubated for 3 days before being lysed and subjected to PCR amplification. Each composition was assayed in two replicate batches. Indels were detected by next generation sequencing of PCR amplicons at the targeted loci and indel percentage was calculated as the fraction of sequencing reads containing insertions or deletions relative to the unedited DMD gene sequence, and are provided in TABLE 17 and FIG. 4. Exemplary raw sequencing data for a sample of data obtained from Example 10 can be seen in FIG. 5, FIG. 6 and FIG. 7. The figures demonstrate that the effector protein of the present disclosure can generate large deletions (e.g., a deletion of greater than 10 nucleotides). Such deletions are substantially larger than deletions generated by Cas9, which can typically only generate a deletion of 1 nucleotide. Advantages associated with such larger deletions include the deletion being more likely to disrupt the target nucleic acid and providing a larger site for HDR insertion of a donor nucleic acid.

TABLE 16
CasPhi.12 Effector Protein (SEQ ID NO: 2) and Guide Nucleic Acid Combinations

| | | Repeat | Spacer | | |
|---|---|---|---|---|---|
| | | Sequence | Sequence | crRNA | Exon |
| Name | PAM | SEQ ID NO | SEQ ID NO | SEQ ID NO | No. |
| T001_CasPhi.12 | TTTA | 740 | 112 | 832 | 44 |
| T002_CasPhi.12 | TTTT | 740 | 113 | 833 | 44 |
| T003_CasPhi.12 | CTTT | 740 | 114 | 834 | 44 |
| T004_CasPhi.12 | ATTA | 740 | 115 | 835 | 44 |
| T005_CasPhi.12 | TTTT | 740 | 116 | 836 | 44 |
| T006_CasPhi.12 | ATTT | 740 | 117 | 837 | 44 |
| T007_CasPhi.12 | ATTT | 740 | 118 | 838 | 44 |
| T008_CasPhi.12 | CTTG | 740 | 119 | 839 | 44 |
| T009_CasPhi.12 | ATTT | 740 | 120 | 840 | 44 |
| T010_CasPhi.12 | ATTT | 740 | 121 | 84 | 44 |
| T011_CasPhi.12 | TTTG | 740 | 122 | 842 | 44 |
| T012_CasPhi.12 | ATTT | 740 | 123 | 843 | 44 |
| T013_CasPhi.12 | GTTT | 740 | 124 | 844 | 44 |
| T014_CasPhi.12 | GTTA | 740 | 125 | 845 | 44 |
| T015_CasPhi.12 | ATTT | 740 | 126 | 846 | 44 |
| T016_CasPhi.12 | TTTT | 740 | 127 | 847 | 44 |
| T017_CasPhi.12 | TTTT | 740 | 128 | 848 | 44 |
| T018_CasPhi.12 | GTTA | 740 | 129 | 849 | 44 |
| T019_CasPhi.12 | CTTT | 740 | 130 | 850 | 44 |
| T020_CasPhi.12 | TTTC | 740 | 131 | 851 | 44 |
| T021_CasPhi.12 | GTTT | 740 | 132 | 852 | 44 |
| T022_CasPhi.12 | CTTG | 740 | 133 | 853 | 44 |
| T023_CasPhi.12 | TTTG | 740 | 134 | 854 | 44 |
| T024_CasPhi.12 | TTTA | 740 | 135 | 855 | 44 |
| T025_CasPhi.12 | ATTA | 740 | 136 | 856 | 44 |
| T026_CasPhi.12 | TTTG | 740 | 137 | 857 | 44 |
| T027_CasPhi.12 | TTTT | 740 | 138 | 858 | 44 |
| T028_CasPhi.12 | TTTA | 740 | 139 | 859 | 44 |
| T029_CasPhi.12 | TTTG | 740 | 140 | 860 | 44 |
| T030_CasPhi.12 | TTTC | 740 | 141 | 861 | 44 |
| T031_CasPhi.12 | TTTC | 740 | 142 | 862 | 44 |
| T032_CasPhi.12 | CTTC | 740 | 143 | 863 | 44 |
| T033_CasPhi.12 | GTTA | 740 | 144 | 864 | 44 |
| T034_CasPhi.12 | ATTT | 740 | 145 | 865 | 44 |
| T035_CasPhi.12 | TTTT | 740 | 146 | 866 | 44 |
| T036_CasPhi.12 | CTTA | 740 | 147 | 867 | 44 |
| T037_CasPhi.12 | TTTA | 740 | 148 | 868 | 44 |
| T038_CasPhi.12 | CTTT | 740 | 149 | 869 | 44 |
| T039_CasPhi.12 | CTTA | 740 | 150 | 870 | 44 |
| T040_CasPhi.12 | TTTA | 740 | 151 | 871 | 44 |
| T041_CasPhi.12 | GTTC | 740 | 152 | 872 | 44 |
| T042_CasPhi.12 | TTTA | 740 | 153 | 873 | 44 |
| T043_CasPhi.12 | CTTT | 740 | 154 | 874 | 44 |
| T044_CasPhi.12 | TTTG | 740 | 155 | 875 | 44 |
| T045_CasPhi.12 | TTTT | 740 | 156 | 876 | 44 |
| T046_CasPhi.12 | CTTT | 740 | 157 | 877 | 44 |
| T047_CasPhi.12 | CTTA | 740 | 158 | 878 | 44 |
| T048_CasPhi.12 | CTTC | 740 | 159 | 879 | 44 |
| T049_CasPhi.12 | TTTT | 740 | 160 | 880 | 44 |
| T050_CasPhi.12 | CTTT | 740 | 161 | 881 | 44 |
| T051_CasPhi.12 | ATTT | 740 | 162 | 882 | 44 |
| T052_CasPhi.12 | ATTG | 740 | 163 | 883 | 44 |
| T053_CasPhi.12 | TTTC | 740 | 164 | 884 | 44 |
| T054_CasPhi.12 | GTTG | 740 | 165 | 885 | 44 |
| T055_CasPhi.12 | CTTA | 740 | 166 | 886 | 44 |
| T056_CasPhi.12 | ATTA | 740 | 167 | 887 | 44 |
| T057_CasPhi.12 | TTTC | 740 | 168 | 888 | 44 |
| T058_CasPhi.12 | ATTT | 740 | 169 | 889 | 44 |
| T059_CasPhi.12 | TTTT | 740 | 170 | 890 | 44 |
| T060_CasPhi.12 | GTTT | 740 | 171 | 891 | 44 |
| T061_CasPhi.12 | GTTG | 740 | 172 | 892 | 44 |
| T062_CasPhi.12 | TTTC | 740 | 173 | 893 | 44 |
| T063_CasPhi.12 | ATTA | 740 | 174 | 894 | 44 |
| T064_CasPhi.12 | CTTT | 740 | 175 | 895 | 44 |
| T065_CasPhi.12 | TTTG | 740 | 176 | 896 | 44 |
| T066_CasPhi.12 | ATTA | 740 | 177 | 897 | 44 |
| T067_CasPhi.12 | TTTG | 740 | 178 | 898 | 44 |
| T068_CasPhi.12 | CTTT | 740 | 179 | 899 | 45 |
| T069_CasPhi.12 | ATTA | 740 | 180 | 900 | 45 |
| T070_CasPhi.12 | TTTT | 740 | 181 | 901 | 45 |
| T071_CasPhi.12 | TTTT | 740 | 182 | 902 | 45 |
| T072_CasPhi.12 | TTTG | 740 | 183 | 903 | 45 |
| T073_CasPhi.12 | TTTG | 740 | 184 | 904 | 45 |
| T074_CasPhi.12 | TTTC | 740 | 185 | 905 | 45 |
| T075_CasPhi.12 | CTTA | 740 | 186 | 906 | 45 |
| T076_CasPhi.12 | CTTT | 740 | 187 | 907 | 45 |
| T077_CasPhi.12 | TTTT | 740 | 188 | 908 | 45 |
| T078_CasPhi.12 | ATTT | 740 | 189 | 909 | 45 |
| T079_CasPhi.12 | TTTC | 740 | 190 | 910 | 45 |
| T080_CasPhi.12 | CTTT | 740 | 191 | 911 | 45 |
| T081_CasPhi.12 | TTTC | 740 | 192 | 912 | 45 |
| T082_CasPhi.12 | GTTT | 740 | 193 | 913 | 45 |
| T083_CasPhi.12 | ATTT | 740 | 194 | 914 | 45 |
| T084_CasPhi.12 | TTTT | 740 | 195 | 915 | 45 |
| T085_CasPhi.12 | TTTG | 740 | 196 | 916 | 45 |
| T086_CasPhi.12 | ATTT | 740 | 197 | 917 | 45 |
| T087_CasPhi.12 | TTTC | 740 | 198 | 918 | 45 |
| T088_CasPhi.12 | GTTG | 740 | 199 | 919 | 45 |
| T089_CasPhi.12 | TTTC | 740 | 200 | 920 | 45 |
| T090_CasPhi.12 | ATTT | 740 | 201 | 921 | 45 |
| T091_CasPhi.12 | GTTT | 740 | 202 | 922 | 45 |
| T092_CasPhi.12 | TTTT | 740 | 203 | 923 | 45 |
| T093_CasPhi.12 | TTTT | 740 | 204 | 924 | 45 |
| T094_CasPhi.12 | TTTG | 740 | 205 | 925 | 45 |
| T095_CasPhi.12 | GTTT | 740 | 206 | 926 | 45 |
| T096_CasPhi.12 | TTTT | 740 | 207 | 927 | 45 |
| T097_CasPhi.12 | TTTT | 740 | 208 | 928 | 45 |
| T098_CasPhi.12 | TTTT | 740 | 209 | 929 | 45 |
| T099_CasPhi.12 | TTTC | 740 | 210 | 930 | 45 |
| T100_CasPhi.12 | TTTA | 740 | 211 | 931 | 45 |
| T101_CasPhi.12 | CTTT | 740 | 212 | 932 | 45 |
| T102_CasPhi.12 | TTTG | 740 | 213 | 933 | 45 |
| T103_CasPhi.12 | TTTG | 740 | 214 | 934 | 45 |
| T104_CasPhi.12 | ATTT | 740 | 215 | 935 | 45 |
| T105_CasPhi.12 | CTTT | 740 | 216 | 936 | 45 |
| T106_CasPhi.12 | TTTT | 740 | 217 | 937 | 45 |
| T107_CasPhi.12 | CTTT | 740 | 218 | 938 | 45 |
| T108_CasPhi.12 | CTTC | 740 | 219 | 939 | 45 |
| T109_CasPhi.12 | ATTC | 740 | 220 | 940 | 45 |
| T110_CasPhi.12 | ATTA | 740 | 221 | 941 | 45 |
| T111_CasPhi.12 | TTTC | 740 | 222 | 942 | 45 |
| T112_CasPhi.12 | ATTA | 740 | 223 | 943 | 45 |
| T113_CasPhi.12 | GTTA | 740 | 224 | 944 | 45 |
| T114_CasPhi.12 | ATTA | 740 | 225 | 945 | 45 |
| T115_CasPhi.12 | CTTT | 740 | 226 | 946 | 45 |
| T116_CasPhi.12 | ATTT | 740 | 227 | 947 | 45 |
| T117_CasPhi.12 | ATTT | 740 | 228 | 948 | 45 |
| T118_CasPhi.12 | GTTG | 740 | 229 | 949 | 45 |
| T119_CasPhi.12 | TTTA | 740 | 230 | 950 | 45 |
| T120_CasPhi.12 | CTTT | 740 | 231 | 951 | 45 |
| T121_CasPhi.12 | TTTA | 740 | 232 | 952 | 45 |
| T122_CasPhi.12 | TTTC | 740 | 233 | 953 | 45 |
| T123_CasPhi.12 | TTTT | 740 | 234 | 954 | 45 |
| T124_CasPhi.12 | ATTA | 740 | 235 | 955 | 45 |
| T125_CasPhi.12 | TTTT | 740 | 236 | 956 | 45 |
| T126_CasPhi.12 | TTTT | 740 | 237 | 957 | 45 |
| T127_CasPhi.12 | TTTC | 740 | 238 | 958 | 45 |
| T128_CasPhi.12 | TTTT | 740 | 239 | 959 | 45 |
| T129_CasPhi.12 | TTTA | 740 | 240 | 960 | 45 |
| T130_CasPhi.12 | ATTA | 740 | 241 | 961 | 45 |
| T131_CasPhi.12 | TTTT | 740 | 242 | 962 | 45 |
| T132_CasPhi.12 | CTTT | 740 | 243 | 963 | 45 |
| T133_CasPhi.12 | TTTC | 740 | 244 | 964 | 45 |
| T134_CasPhi.12 | CTTT | 740 | 245 | 965 | 45 |
| T135_CasPhi.12 | TTTA | 740 | 246 | 966 | 45 |
| T136_CasPhi.12 | GTTC | 740 | 247 | 967 | 45 |
| T137_CasPhi.12 | CTTT | 740 | 248 | 968 | 45 |
| T138_CasPhi.12 | GTTT | 740 | 249 | 969 | 45 |
| T139_CasPhi.12 | TTTC | 740 | 250 | 970 | 45 |
| T140_CasPhi.12 | TTTT | 740 | 251 | 971 | 45 |
| T141_CasPhi.12 | TTTA | 740 | 252 | 972 | 45 |
| T142_CasPhi.12 | ATTT | 740 | 253 | 973 | 45 |
| T143_CasPhi.12 | ATTC | 740 | 254 | 974 | 45 |
| T144_CasPhi.12 | GTTG | 740 | 255 | 975 | 45 |
| T145_CasPhi.12 | CTTA | 740 | 256 | 976 | 45 |
| T146_CasPhi.12 | TTTT | 740 | 257 | 977 | 45 |
| T147_CasPhi.12 | CTTT | 740 | 258 | 978 | 45 |
| T148_CasPhi.12 | CTTA | 740 | 259 | 979 | 45 |
| T149_CasPhi.12 | CTTT | 740 | 260 | 980 | 45 |
| T150_CasPhi.12 | TTTC | 740 | 261 | 981 | 45 |
| T151_CasPhi.12 | GTTA | 740 | 262 | 982 | 50 |
| T152_CasPhi.12 | ATTA | 740 | 263 | 983 | 50 |
| T153_CasPhi.12 | CTTA | 740 | 264 | 984 | 50 |
| T154_CasPhi.12 | GTTA | 740 | 265 | 985 | 50 |
| T155_CasPhi.12 | ATTC | 740 | 266 | 986 | 50 |
| T156_CasPhi.12 | ATTC | 740 | 267 | 987 | 50 |
| T157_CasPhi.12 | CTTC | 740 | 268 | 988 | 50 |
| T158_CasPhi.12 | TTTA | 740 | 269 | 989 | 50 |
| T159_CasPhi.12 | CTTT | 740 | 270 | 990 | 50 |
| T160_CasPhi.12 | ATTC | 740 | 271 | 991 | 50 |
| T161_CasPhi.12 | ATTC | 740 | 272 | 992 | 50 |
| T162_CasPhi.12 | ATTT | 740 | 273 | 993 | 50 |
| T163_CasPhi.12 | CTTT | 740 | 274 | 994 | 50 |
| T164_CasPhi.12 | CTTT | 740 | 275 | 995 | 50 |
| T165_CasPhi.12 | TTTC | 740 | 276 | 996 | 50 |
| T166_CasPhi.12 | ATTA | 740 | 277 | 997 | 50 |
| T167_CasPhi.12 | TTTT | 740 | 278 | 998 | 50 |
| T168_CasPhi.12 | TTTG | 740 | 279 | 999 | 50 |
| T169_CasPhi.12 | CTTA | 740 | 280 | 1000 | 50 |
| T170_CasPhi.12 | TTTA | 740 | 281 | 1001 | 50 |
| T171_CasPhi.12 | GTTA | 740 | 282 | 1002 | 50 |
| T172_CasPhi.12 | CTTC | 740 | 283 | 1003 | 50 |
| T173_CasPhi.12 | TTTG | 740 | 284 | 1004 | 50 |
| T174_CasPhi.12 | ATTA | 740 | 285 | 1005 | 50 |
| T175_CasPhi.12 | CTTC | 740 | 286 | 1006 | 50 |
| T176_CasPhi.12 | CTTT | 740 | 287 | 1007 | 50 |
| T177_CasPhi.12 | CTTC | 740 | 288 | 1008 | 50 |
| T178_CasPhi.12 | TTTA | 740 | 289 | 1009 | 50 |
| T179_CasPhi.12 | TTTA | 740 | 290 | 1010 | 50 |
| T180_CasPhi.12 | GTTC | 740 | 291 | 1011 | 50 |
| T181_CasPhi.12 | ATTA | 740 | 292 | 1012 | 50 |
| T182_CasPhi.12 | ATTT | 740 | 293 | 1013 | 50 |
| T183_CasPhi.12 | TTTA | 740 | 294 | 1014 | 50 |
| T184_CasPhi.12 | TTTT | 740 | 295 | 1015 | 50 |
| T185_CasPhi.12 | ATTT | 740 | 296 | 1016 | 50 |
| T186_CasPhi.12 | GTTA | 740 | 297 | 1017 | 50 |
| T187_CasPhi.12 | ATTC | 740 | 298 | 1018 | 50 |
| T188_CasPhi.12 | ATTA | 740 | 299 | 1019 | 50 |
| T189_CasPhi.12 | ATTT | 740 | 300 | 1020 | 50 |
| T190_CasPhi.12 | ATTG | 740 | 301 | 1021 | 50 |
| T191_CasPhi.12 | ATTA | 740 | 302 | 1022 | 50 |
| T192_CasPhi.12 | TTTC | 740 | 303 | 1023 | 50 |
| T193_CasPhi.12 | CTTA | 740 | 304 | 1024 | 50 |
| T194_CasPhi.12 | ATTG | 740 | 305 | 1025 | 50 |
| T195_CasPhi.12 | TTTG | 740 | 306 | 1026 | 50 |
| T196_CasPhi.12 | CTTT | 740 | 307 | 1027 | 50 |
| T197_CasPhi.12 | TTTT | 740 | 308 | 1028 | 50 |
| T198_CasPhi.12 | CTTT | 740 | 309 | 1029 | 50 |
| T199_CasPhi.12 | TTTT | 740 | 310 | 1030 | 50 |
| T200_CasPhi.12 | TTTT | 740 | 311 | 1031 | 50 |
| T201_CasPhi.12 | ATTT | 740 | 312 | 1032 | 50 |
| T202_CasPhi.12 | TTTG | 740 | 313 | 1033 | 50 |
| T203_CasPhi.12 | CTTT | 740 | 314 | 1034 | 50 |
| T204_CasPhi.12 | CTTT | 740 | 315 | 1035 | 50 |
| T205_CasPhi.12 | TTTG | 740 | 316 | 1036 | 50 |
| T206_CasPhi.12 | TTTG | 740 | 317 | 1037 | 50 |
| T207_CasPhi.12 | TTTC | 740 | 318 | 1038 | 50 |
| T208_CasPhi.12 | GTTA | 740 | 319 | 1039 | 50 |
| T209_CasPhi.12 | ATTC | 740 | 320 | 1040 | 50 |
| T210_CasPhi.12 | ATTT | 740 | 321 | 1041 | 50 |
| T211_CasPhi.12 | ATTG | 740 | 322 | 1042 | 50 |
| T212_CasPhi.12 | TTTG | 740 | 323 | 1043 | 50 |
| T213_CasPhi.12 | CTTC | 740 | 324 | 1044 | 50 |
| T214_CasPhi.12 | GTTT | 740 | 325 | 1045 | 50 |
| T215_CasPhi.12 | ATTT | 740 | 326 | 1046 | 50 |
| T216_CasPhi.12 | GTTG | 740 | 327 | 1047 | 50 |
| T217_CasPhi.12 | GTTC | 740 | 328 | 1048 | 50 |
| T218_CasPhi.12 | TTTT | 740 | 329 | 1049 | 50 |
| T219_CasPhi.12 | CTTT | 740 | 330 | 1050 | 50 |
| T220_CasPhi.12 | TTTA | 740 | 331 | 1051 | 50 |
| T221_CasPhi.12 | TTTA | 740 | 332 | 1052 | 50 |
| T222_CasPhi.12 | TTTC | 740 | 333 | 1053 | 50 |
| T223_CasPhi.12 | ATTC | 740 | 334 | 1054 | 50 |
| T224_CasPhi.12 | ATTG | 740 | 335 | 1055 | 50 |
| T225_CasPhi.12 | TTTG | 740 | 336 | 1056 | 50 |
| T226_CasPhi.12 | TTTA | 740 | 337 | 1057 | 51 |
| T227_CasPhi.12 | TTTT | 740 | 338 | 1058 | 51 |
| T228_CasPhi.12 | GTTT | 740 | 339 | 1059 | 51 |
| T229_CasPhi.12 | ATTT | 740 | 340 | 1060 | 51 |
| T230_CasPhi.12 | ATTT | 740 | 341 | 1061 | 51 |
| T231_CasPhi.12 | TTTT | 740 | 342 | 1062 | 51 |
| T232_CasPhi.12 | TTTG | 740 | 343 | 1063 | 51 |
| T233_CasPhi.12 | ATTG | 740 | 344 | 1064 | 51 |
| T234_CasPhi.12 | ATTG | 740 | 345 | 1065 | 51 |
| T235_CasPhi.12 | TTTG | 740 | 346 | 1066 | 51 |
| T236_CasPhi.12 | TTTT | 740 | 347 | 1067 | 51 |
| T237_CasPhi.12 | ATTG | 740 | 348 | 1068 | 51 |
| T238_CasPhi.12 | ATTA | 740 | 349 | 1069 | 51 |
| T239_CasPhi.12 | ATTT | 740 | 350 | 1070 | 51 |
| T240_CasPhi.12 | TTTT | 740 | 351 | 1071 | 51 |
| T241_CasPhi.12 | TTTG | 740 | 352 | 1072 | 51 |
| T242_CasPhi.12 | TTTT | 740 | 353 | 1073 | 51 |
| T243_CasPhi.12 | ATTT | 740 | 354 | 1074 | 51 |
| T244_CasPhi.12 | TTTA | 740 | 355 | 1075 | 51 |
| T245_CasPhi.12 | GTTA | 740 | 356 | 1076 | 51 |
| T246_CasPhi.12 | TTTT | 740 | 357 | 1077 | 51 |
| T247_CasPhi.12 | ATTG | 740 | 358 | 1078 | 51 |
| T248_CasPhi.12 | CTTT | 740 | 359 | 1079 | 51 |
| T249_CasPhi.12 | CTTT | 740 | 360 | 1080 | 51 |
| T250_CasPhi.12 | CTTA | 740 | 361 | 1081 | 51 |
| T251_CasPhi.12 | TTTA | 740 | 362 | 1082 | 51 |
| T252_CasPhi.12 | CTTA | 740 | 363 | 1083 | 51 |
| T253_CasPhi.12 | GTTA | 740 | 364 | 1084 | 51 |
| T254_CasPhi.12 | ATTG | 740 | 365 | 1085 | 51 |
| T255_CasPhi.12 | TTTG | 740 | 366 | 1086 | 51 |
| T256_CasPhi.12 | TTTT | 740 | 367 | 1087 | 51 |
| T257_CasPhi.12 | TTTC | 740 | 368 | 1088 | 51 |
| T258_CasPhi.12 | TTTG | 740 | 369 | 1089 | 51 |
| T259_CasPhi.12 | ATTA | 740 | 370 | 1090 | 51 |
| T260_CasPhi.12 | CTTC | 740 | 371 | 1091 | 51 |
| T261_CasPhi.12 | CTTT | 740 | 372 | 1092 | 51 |
| T262_CasPhi.12 | TTTT | 740 | 373 | 1093 | 51 |
| T263_CasPhi.12 | TTTT | 740 | 374 | 1094 | 51 |
| T264_CasPhi.12 | TTTG | 740 | 375 | 1095 | 51 |
| T265_CasPhi.12 | CTTC | 740 | 376 | 1096 | 51 |
| T266_CasPhi.12 | TTTC | 740 | 377 | 1097 | 51 |
| T267_CasPhi.12 | CTTG | 740 | 378 | 1098 | 51 |
| T268_CasPhi.12 | GTTT | 740 | 379 | 1099 | 51 |
| T269_CasPhi.12 | TTTC | 740 | 380 | 1100 | 51 |
| T270_CasPhi.12 | TTTT | 740 | 381 | 1101 | 51 |
| T271_CasPhi.12 | TTTC | 740 | 382 | 1102 | 51 |
| T272_CasPhi.12 | CTTT | 740 | 383 | 1103 | 51 |
| T273_CasPhi.12 | TTTT | 740 | 384 | 1104 | 51 |
| T274_CasPhi.12 | TTTT | 740 | 385 | 1105 | 51 |
| T275_CasPhi.12 | ATTT | 740 | 386 | 1106 | 51 |
| T276_CasPhi.12 | ATTT | 740 | 387 | 1107 | 51 |
| T277_CasPhi.12 | GTTG | 740 | 388 | 1108 | 51 |
| T278_CasPhi.12 | TTTC | 740 | 389 | 1109 | 51 |
| T279_CasPhi.12 | CTTG | 740 | 390 | 1110 | 51 |
| T280_CasPhi.12 | CTTC | 740 | 391 | 1111 | 51 |
| T281_CasPhi.12 | TTTC | 740 | 392 | 1112 | 51 |
| T282_CasPhi.12 | ATTT | 740 | 393 | 1113 | 51 |
| T283_CasPhi.12 | CTTT | 740 | 394 | 1114 | 51 |
| T284_CasPhi.12 | TTTT | 740 | 395 | 1115 | 51 |
| T285_CasPhi.12 | TTTA | 740 | 396 | 1116 | 51 |
| T286_CasPhi.12 | GTTT | 740 | 397 | 1117 | 51 |
| T287_CasPhi.12 | ATTA | 740 | 398 | 1118 | 51 |
| T288_CasPhi.12 | TTTC | 740 | 399 | 1119 | 51 |
| T289_CasPhi.12 | GTTC | 740 | 400 | 1120 | 51 |
| T290_CasPhi.12 | TTTT | 740 | 401 | 1121 | 51 |
| T291_CasPhi.12 | CTTA | 740 | 402 | 1122 | 51 |
| T292_CasPhi.12 | GTTT | 740 | 403 | 1123 | 51 |
| T293_CasPhi.12 | TTTT | 740 | 404 | 1124 | 51 |
| T294_CasPhi.12 | TTTC | 740 | 405 | 1125 | 51 |
| T295_CasPhi.12 | TTTA | 740 | 406 | 1126 | 51 |
| T296_CasPhi.12 | TTTT | 740 | 407 | 1127 | 51 |
| T297_CasPhi.12 | TTTG | 740 | 408 | 1128 | 51 |
| T298_CasPhi.12 | GTTG | 740 | 409 | 1129 | 51 |
| T299_CasPhi.12 | ATTT | 740 | 410 | 1130 | 51 |
| T300_CasPhi.12 | ATTG | 740 | 411 | 1131 | 51 |
| T301_CasPhi.12 | ATTT | 740 | 412 | 1132 | 51 |
| T302_CasPhi.12 | TTTA | 740 | 413 | 1133 | 51 |
| T303_CasPhi.12 | CTTC | 740 | 414 | 1134 | 51 |
| T304_CasPhi.12 | TTTT | 740 | 415 | 1135 | 51 |
| T305_CasPhi.12 | CTTT | 740 | 416 | 1136 | 51 |
| T306_CasPhi.12 | TTTA | 740 | 417 | 1137 | 51 |
| T307_CasPhi.12 | CTTT | 740 | 418 | 1138 | 51 |
| T308_CasPhi.12 | TTTT | 740 | 419 | 1139 | 51 |
| T309_CasPhi.12 | TTTT | 740 | 420 | 1140 | 51 |
| T310_CasPhi.12 | TTTT | 740 | 421 | 1141 | 51 |
| T311_CasPhi.12 | GTTT | 740 | 422 | 1142 | 51 |
| T312_CasPhi.12 | CTTC | 740 | 423 | 1143 | 51 |
| T313_CasPhi.12 | TTTC | 740 | 424 | 1144 | 51 |
| T314_CasPhi.12 | GTTC | 740 | 425 | 1145 | 53 |
| T315_CasPhi.12 | ATTG | 740 | 426 | 1146 | 53 |
| T316_CasPhi.12 | TTTA | 740 | 427 | 1147 | 53 |
| T317_CasPhi.12 | ATTC | 740 | 428 | 1148 | 53 |
| T318_CasPhi.12 | GTTG | 740 | 429 | 1149 | 53 |
| T319_CasPhi.12 | CTTC | 740 | 430 | 1150 | 53 |
| T320_CasPhi.12 | TTTA | 740 | 431 | 1151 | 53 |
| T321_CasPhi.12 | ATTA | 740 | 432 | 1152 | 53 |
| T322_CasPhi.12 | ATTC | 740 | 433 | 1153 | 53 |
| T323_CasPhi.12 | ATTT | 740 | 434 | 1154 | 53 |
| T324_CasPhi.12 | CTTC | 740 | 435 | 1155 | 53 |
| T325_CasPhi.12 | GTTT | 740 | 436 | 1156 | 53 |
| T326_CasPhi.12 | ATTC | 740 | 437 | 1157 | 53 |
| T327_CasPhi.12 | CTTT | 740 | 438 | 1158 | 53 |
| T328_CasPhi.12 | GTTA | 740 | 439 | 1159 | 53 |
| T329_CasPhi.12 | TTTT | 740 | 440 | 1160 | 53 |
| T330_CasPhi.12 | GTTA | 740 | 441 | 1161 | 53 |
| T331_CasPhi.12 | ATTC | 740 | 442 | 1162 | 53 |
| T332_CasPhi.12 | GTTG | 740 | 443 | 1163 | 53 |
| T333_CasPhi.12 | TTTA | 740 | 444 | 1164 | 53 |
| T334_CasPhi.12 | TTTC | 740 | 445 | 1165 | 53 |
| T335_CasPhi.12 | TTTT | 740 | 446 | 1166 | 53 |
| T336_CasPhi.12 | TTTA | 740 | 447 | 1167 | 53 |
| T337_CasPhi.12 | CTTT | 740 | 448 | 1168 | 53 |
| T338_CasPhi.12 | TTTT | 740 | 449 | 1169 | 53 |
| T339_CasPhi.12 | TTTT | 740 | 450 | 1170 | 53 |
| T340_CasPhi.12 | ATTT | 740 | 451 | 1171 | 53 |
| T341_CasPhi.12 | TTTA | 740 | 452 | 1172 | 53 |
| T342_CasPhi.12 | TTTT | 740 | 453 | 1173 | 53 |
| T343_CasPhi.12 | TTTT | 740 | 454 | 1174 | 53 |
| T344_CasPhi.12 | TTTG | 740 | 455 | 1175 | 53 |
| T345_CasPhi.12 | TTTC | 740 | 456 | 1176 | 53 |
| T346_CasPhi.12 | ATTT | 740 | 457 | 1177 | 53 |
| T347_CasPhi.12 | ATTT | 740 | 458 | 1178 | 53 |
| T348_CasPhi.12 | ATTT | 740 | 459 | 1179 | 53 |
| T349_CasPhi.12 | TTTA | 740 | 460 | 1180 | 53 |
| T350_CasPhi.12 | TTTA | 740 | 461 | 1181 | 53 |
| T351_CasPhi.12 | ATTT | 740 | 462 | 1182 | 53 |
| T352_CasPhi.12 | ATTT | 740 | 463 | 1183 | 53 |
| T353_CasPhi.12 | CTTT | 740 | 464 | 1184 | 53 |
| T354_CasPhi.12 | GTTC | 740 | 465 | 1185 | 53 |
| T355_CasPhi.12 | TTTG | 740 | 466 | 1186 | 53 |
| T356_CasPhi.12 | GTTA | 740 | 467 | 1187 | 53 |
| T357_CasPhi.12 | ATTA | 740 | 468 | 1188 | 53 |
| T358_CasPhi.12 | GTTA | 740 | 469 | 1189 | 53 |
| T359_CasPhi.12 | ATTT | 740 | 470 | 1190 | 53 |
| T360_CasPhi.12 | ATTA | 740 | 471 | 1191 | 53 |
| T361_CasPhi.12 | TTTT | 740 | 472 | 1192 | 53 |
| T362_CasPhi.12 | TTTC | 740 | 473 | 1193 | 53 |
| T363_CasPhi.12 | TTTA | 740 | 474 | 1194 | 53 |
| T364_CasPhi.12 | CTTT | 740 | 475 | 1195 | 53 |
| T365_CasPhi.12 | TTTA | 740 | 476 | 1196 | 53 |
| T366_CasPhi.12 | ATTA | 740 | 477 | 1197 | 53 |
| T367_CasPhi.12 | ATTA | 740 | 478 | 1198 | 53 |
| T368_CasPhi.12 | GTTA | 740 | 479 | 1199 | 53 |
| T369_CasPhi.12 | ATTT | 740 | 480 | 1200 | 53 |
| T370_CasPhi.12 | TTTT | 740 | 481 | 1201 | 53 |
| T371_CasPhi.12 | ATTT | 740 | 482 | 1202 | 53 |
| T372_CasPhi.12 | TTTA | 740 | 483 | 1203 | 53 |
| T373_CasPhi.12 | ATTT | 740 | 484 | 1204 | 53 |
| T374_CasPhi.12 | TTTA | 740 | 485 | 1205 | 53 |
| T375_CasPhi.12 | CTTT | 740 | 486 | 1206 | 53 |
| T376_CasPhi.12 | TTTA | 740 | 487 | 1207 | 53 |
| T377_CasPhi.12 | TTTG | 740 | 488 | 1208 | 53 |
| T378_CasPhi.12 | ATTT | 740 | 489 | 1209 | 53 |
| T379_CasPhi.12 | TTTT | 740 | 490 | 1210 | 53 |
| T380_CasPhi.12 | GTTT | 740 | 491 | 1211 | 53 |
| T381_CasPhi.12 | ATTA | 740 | 492 | 1212 | 53 |
| T382_CasPhi.12 | TTTA | 740 | 493 | 1213 | 53 |
| T383_CasPhi.12 | TTTT | 740 | 494 | 1214 | 53 |
| T384_CasPhi.12 | CTTT | 740 | 495 | 1215 | 53 |
| T385_CasPhi.12 | TTTT | 740 | 496 | 1216 | 53 |
| T386_CasPhi.12 | GTTT | 740 | 497 | 1217 | 53 |
| T387_CasPhi.12 | TTTC | 740 | 498 | 1218 | 53 |
| T388_CasPhi.12 | ATTT | 740 | 499 | 1219 | 53 |
| T389_CasPhi.12 | ATTG | 740 | 500 | 1220 | 53 |
| T390_CasPhi.12 | CTTG | 740 | 501 | 1221 | 53 |
| T391_CasPhi.12 | TTTT | 740 | 502 | 1222 | 53 |
| T392_CasPhi.12 | TTTA | 740 | 503 | 1223 | 53 |
| T393_CasPhi.12 | GTTA | 740 | 504 | 1224 | 53 |
| T394_CasPhi.12 | TTTC | 740 | 505 | 1225 | 53 |

TABLE 17
Indel activity of Effector Protein (SEQ ID NO: 2)
and Guide Nucleic Acid Combinations of TABLE 16

| | % INDEL | % INDEL | % INDEL |
|---|---|---|---|
| NAME | REP1 | REP2 | AVERAGE |
| T001 | 2.655850076 | 1.320121002 | 1.987985539 |
| T002 | 1.295630468 | 0.915495445 | 1.105562956 |
| T003 | 0.001751932 | 0.007340976 | 0.004546454 |
| T004 | 0.719140638 | 0.648331437 | 0.683736037 |
| T005 | 11.11370935 | 15.02750533 | 13.07060734 |
| T006 | 0.02065177 | 0.047334902 | 0.033993336 |
| T007 | 0.85208186 | 0.832433287 | 0.842257573 |
| T008 | 0.006335252 | 0.00651736 | 0.006426306 |
| T009 | 0.617635519 | 2.934017569 | 1.775826544 |
| T010 | 0.005285492 | 0.006789109 | 0.0060373 |
| T011 | 1.22642605 | 1.533960575 | 1.380193312 |
| T012 | 0.013354573 | 0.02399225 | 0.018673412 |
| T013 | 0.022168701 | 0.021379098 | 0.0217739 |
| T014 | 0.011047211 | 0.019463554 | 0.015255383 |
| T015 | 0.032921449 | 0.027880086 | 0.030400768 |
| T016 | 0.388413702 | 0.438153965 | 0.413283833 |
| T017 | 0.025033012 | 0.065223063 | 0.045128038 |
| T018 | 1.024017258 | 1.167605941 | 1.095811599 |
| T019 | 0.3398164 | 0.286234515 | 0.313025457 |
| T020 | 0.018886633 | 0.01215389 | 0.015520262 |
| T021 | 0.02367907 | 0.028900198 | 0.026289634 |
| T022 | 6.865374661 | 9.32717848 | 8.09627657 |
| T023 | 0.01413492 | 0.033272264 | 0.023703592 |
| T024 | 0.099842377 | 0.168157542 | 0.133999959 |
| T025 | 0.319672288 | 0.271228285 | 0.295450286 |
| T026 | 0.408438839 | 1.123243442 | 0.765841141 |
| T027 | 0.027820077 | 0.033461855 | 0.030640966 |
| T028 | 0.034480086 | 0.020448668 | 0.027464377 |
| T029 | 0.326102576 | 0.19859413 | 0.262348353 |
| T030 | 0.027041977 | 0.026856346 | 0.026949161 |
| T031 | 1.485748939 | 2.280037362 | 1.882893151 |
| T032 | 0.033981964 | 0.033497428 | 0.033739696 |
| T033 | 10.55841075 | 22.2581956 | 16.40830318 |
| T034 | 14.29313321 | 22.72555572 | 18.50934446 |
| T035 | 31.66568032 | 34.62188886 | 33.14378459 |
| T036 | 1.5536357 | 1.843364831 | 1.698500266 |
| T037 | 0.019144283 | 0.106587158 | 0.062865721 |
| T038 | 0.201028906 | 0.191302451 | 0.196165678 |
| T039 | 0.009306272 | 0.012328472 | 0.010817372 |
| T040 | 0.010194898 | 0.02317382 | 0.016684359 |
| T041 | 0.011649877 | 0.01411928 | 0.012884579 |
| T042 | 0.030033234 | 0.135281793 | 0.082657514 |
| T043 | 0.01800048 | 0.186484713 | 0.102242596 |
| T044 | 0.403038623 | 0.365645351 | 0.384341987 |
| T045 | 0.00682973 | 0.010582011 | 0.00870587 |
| T046 | 7.862464183 | 13.601158 | 10.73181109 |
| T047 | 0.061150976 | 0.081489498 | 0.071320237 |
| T048 | 0.015037947 | 0.023136779 | 0.019087363 |
| T049 | 0.007995283 | 0.012344503 | 0.010169893 |
| T050 | 0.014802849 | 0.022487459 | 0.018645154 |
| T051 | 0.01795668 | 0.02134886 | 0.01965277 |
| T052 | 0.007808285 | 0.028427404 | 0.018117845 |
| T053 | 0.217628921 | 0.891375059 | 0.55450199 |
| T054 | 0.022891153 | 0.017664984 | 0.020278068 |
| T055 | 0.186955922 | 1.061186387 | 0.624071155 |
| T056 | 7.5932035 | 13.53322171 | 10.56321261 |
| T057 | 0.015133014 | 0.012323016 | 0.013728015 |
| T058 | 0.023225926 | 0.039804247 | 0.031515087 |
| T059 | 0.014049525 | 0.008895286 | 0.011472405 |
| T060 | 0.009594021 | 0.011357558 | 0.01047579 |
| T061 | 0.022565305 | 0.022167065 | 0.022366185 |
| T062 | 0.013582701 | 0.016224168 | 0.014903434 |
| T063 | 0.019857029 | 0.021866077 | 0.020861553 |
| T064 | 0.032785215 | 0.029531321 | 0.031158268 |
| T065 | 0.020289832 | 0.020838009 | 0.02056392 |
| T066 | 0.013730422 | 0.014123914 | 0.013927168 |
| T067 | 0.073391775 | 0.081679808 | 0.077535791 |
| T068 | 13.53588593 | 33.51473987 | 23.5253129 |
| T069 | 0.901956772 | 1.54635779 | 1.224157281 |
| T070 | 0.962649004 | 1.164161401 | 1.063405202 |
| T071 | 10.43105767 | 14.41342002 | 12.42223885 |
| T072 | 9.431804272 | 18.7731081 | 14.10245619 |
| T073 | 0.012813089 | 0.017800806 | 0.015306948 |
| T074 | 0.017361865 | 0.010309336 | 0.0138356 |
| T075 | 0.024194797 | 0.01583242 | 0.020013609 |
| T076 | 0.002579218 | 0.002649748 | 0.002614483 |
| T077 | 0.006061137 | 0.007763975 | 0.006912556 |
| T078 | 3.004276011 | 5.670490878 | 4.337383445 |
| T079 | 0.035850248 | 0.016549213 | 0.026199731 |
| T080 | 0.00468653 | 0.00434549 | 0.00451601 |
| T081 | 0.004394549 | 0.003654097 | 0.004024323 |
| T082 | 0.002075205 | 0.051657019 | 0.026866112 |
| T083 | 0.009972695 | 0.013781248 | 0.011876972 |
| T084 | 1.493166584 | 1.328317811 | 1.410742197 |
| T085 | 0.028632539 | 0.041263092 | 0.034947815 |
| T086 | 0.033400955 | 0.027394604 | 0.030397779 |
| T087 | 0.120615919 | 0.322614108 | 0.221615014 |
| T088 | 0.005007124 | 0.034323123 | 0.019665123 |
| T089 | 0.004320269 | 0.017826023 | 0.011073146 |
| T090 | 0.00232311 | 0.004571526 | 0.003447318 |
| T091 | 0.024419223 | 0.022142509 | 0.023280866 |
| T092 | 0.004482218 | 0.010848377 | 0.007665297 |
| T093 | 0.036550322 | 0.199674729 | 0.118112525 |
| T094 | 0.891092859 | 0.938969159 | 0.915031009 |
| T095 | 0.088106388 | 0.087810668 | 0.087958528 |
| T096 | 0.039619101 | 0.042237485 | 0.040928293 |
| T097 | 0.014048658 | 0.007948553 | 0.010998605 |
| T098 | 0.008512398 | 0.01458296 | 0.011547679 |
| T099 | 0.047735901 | 0.037625289 | 0.042680595 |
| T100 | 0.094827851 | 0.06570609 | 0.080266971 |
| T101 | 0.008287407 | 0.029395284 | 0.018841346 |
| T102 | 0.01820715 | 0.018081749 | 0.018144449 |
| T103 | 0.001440926 | 0.001789549 | 0.001615238 |
| T104 | 0.009074804 | 0.009208071 | 0.009141437 |
| T105 | 0.03266009 | 0.035479044 | 0.034069567 |
| T106 | 0.000783533 | 0.004057495 | 0.002420514 |
| T107 | 0.012413108 | 0.010667591 | 0.01154035 |
| T108 | 0.121818933 | 0.134115837 | 0.127967385 |
| T109 | 0.02452862 | 0.311155376 | 0.167841998 |
| T110 | 0.004969356 | 0.011599313 | 0.008284334 |
| T111 | 0.080357478 | 0.478987833 | 0.279672655 |
| T112 | 0.012289542 | 0.111421295 | 0.061855418 |
| T113 | 0.032023313 | 0.030855299 | 0.031439306 |
| T114 | 0.004506856 | 0.322541268 | 0.163524062 |
| T115 | 0.005724037 | 0.100568921 | 0.053146479 |
| T116 | 0.026452557 | 7.920559162 | 3.973505859 |
| T117 | 0.004572608 | 0.003194973 | 0.003883791 |
| T118 | 1.300476087 | 1.549082412 | 1.424779249 |
| T119 | 14.85191418 | 9.618090295 | 12.23500224 |
| T120 | 0.007094022 | 0.011165957 | 0.00912999 |
| T121 | 0.01059024 | 0.0074471 | 0.00901867 |
| T122 | 0.012018834 | 0.016168682 | 0.014093758 |
| T123 | 0.003591481 | 0.005284274 | 0.004437877 |
| T124 | 0.013028346 | 0.01191721 | 0.012472778 |
| T125 | 0.000662361 | 0.000683875 | 0.000673118 |
| T126 | 0.006934837 | 0.043683321 | 0.025309079 |
| T127 | 0.083596202 | 0.539264161 | 0.311430181 |
| T128 | 0.010576207 | 0.015015624 | 0.012795916 |
| T129 | 0.006464516 | 0.005246067 | 0.005855291 |
| T130 | 0.020979089 | 0.155146368 | 0.088062729 |
| T131 | 0.086882105 | 0.314019305 | 0.200450705 |
| T132 | 4.62865088 | 12.27406458 | 8.451357728 |
| T133 | 0.00273127 | 0.00218841 | 0.00245984 |
| T134 | 0.003530097 | 0.007475704 | 0.0055029 |
| T135 | 0.019842113 | 0.562000795 | 0.290921454 |
| T136 | 0.022117683 | 0.026917607 | 0.024517645 |
| T137 | 0.005752857 | 0.003975886 | 0.004864372 |
| T138 | 0 | 0.002187715 | 0.001093857 |
| T139 | 0.003553092 | 0.007219205 | 0.005386148 |
| T140 | 0.00482281 | 0.002010343 | 0.003416577 |
| T141 | 0.003687384 | 0.004913904 | 0.004300644 |
| T142 | 0.005351756 | 0.01388509 | 0.009618423 |
| T143 | 0.083627932 | 0.148714162 | 0.116171047 |
| T144 | 0.779558071 | 0.202501489 | 0.49102978 |
| T145 | 0.013763978 | 0.016562575 | 0.015163277 |
| T146 | 0.00389952 | 0.005363733 | 0.004631626 |
| T147 | 0.002968655 | 0.007607905 | 0.00528828 |
| T148 | 0.004356615 | 0.005179986 | 0.004768301 |
| T149 | 0.002175389 | 0.004008087 | 0.003091738 |
| T150 | 0.004776842 | 0.006028 | 0.005402421 |
| T151 | 0.005188821 | 0.008839063 | 0.007013942 |
| T152 | 0.909319223 | 0.291625913 | 0.600472568 |
| T153 | 0.003806582 | 0.003614463 | 0.003710523 |
| T154 | 0.422362859 | 0.83797765 | 0.630170255 |
| T155 | 0.02077395 | 0.023618 | 0.022195975 |
| T156 | 0.029469838 | 0.031081361 | 0.030275599 |
| T157 | 0.013478906 | 0.01596749 | 0.014723198 |
| T158 | 0.030711061 | 0.025320344 | 0.028015702 |
| T159 | 0.022682053 | 0.01706339 | 0.019872722 |
| T160 | 0.004161402 | 0.006732647 | 0.005447024 |
| T161 | 0.014544646 | 0.054118061 | 0.034331353 |
| T162 | 0.008973182 | 0.007889473 | 0.008431328 |
| T163 | 3.141969098 | 3.981917991 | 3.561943544 |
| T164 | 0.019269679 | 0.017031034 | 0.018150357 |
| T165 | 0.007181361 | 0.114303033 | 0.060742197 |
| T166 | 0.003388854 | 0.003951587 | 0.003670221 |
| T167 | 0.017804456 | 0.023578662 | 0.020691559 |
| T168 | 0.017487023 | 0.012759491 | 0.015123257 |
| T169 | 0.005026253 | 0.00303829 | 0.004032271 |
| T170 | 0.201798775 | 0.017990113 | 0.109894444 |
| T171 | 0.00062916 | 0.000719295 | 0.000674228 |
| T172 | 0.004216918 | 0.006759443 | 0.005488181 |
| T173 | 0.002853372 | 0.004264792 | 0.003559082 |
| T174 | 0.088191631 | 0.119861657 | 0.104026644 |
| T175 | 0.020084705 | 0.020568702 | 0.020326703 |
| T176 | 0.005472664 | 0.183191191 | 0.094331927 |
| T177 | 2.801668307 | 6.844306152 | 4.82298723 |
| T178 | 0.4384324 | 1.048629258 | 0.743530829 |
| T179 | 0.541383668 | 1.140329389 | 0.840856528 |
| T180 | 0.001353656 | 0.069901242 | 0.035627449 |
| T181 | 0.003136249 | 0.004828052 | 0.003982151 |
| T182 | 0.024255851 | 0.023538906 | 0.023897379 |
| T183 | 0.029423554 | 0.02061139 | 0.025017472 |
| T184 | 0.027741276 | 0.021935653 | 0.024838464 |
| T185 | 0.004123603 | 0.005284983 | 0.004704293 |
| T186 | 0.02431927 | 0.016434047 | 0.020376658 |
| T187 | 0.033669078 | 0.020491032 | 0.027080055 |
| T188 | 0.029041064 | 0.028196386 | 0.028618725 |
| T189 | 0.003006596 | 0.019585325 | 0.011295961 |
| T190 | 0.037322098 | 0.034811083 | 0.03606659 |
| T191 | 0.013004193 | 0.015125561 | 0.014064877 |
| T192 | 0.015006275 | 0.014879845 | 0.01494306 |
| T193 | 8.21797094 | 18.05008404 | 13.13402749 |
| T194 | 21.42410211 | 37.05227191 | 29.23818701 |
| T195 | 3.885372002 | 3.005553158 | 3.44546258 |
| T196 | 0.893740641 | 0.360200626 | 0.626970633 |
| T197 | 0.004296769 | 0.033313108 | 0.018804938 |
| T198 | 6.228616769 | 8.769460231 | 7.4990385 |
| T199 | 0.074872888 | 0.168103433 | 0.121488161 |
| T200 | 0.357517132 | 1.142936526 | 0.750226829 |
| T201 | 0.639708513 | 1.099351088 | 0.8695298 |
| T202 | 0.876431236 | 0.552584603 | 0.71450792 |
| T203 | 2.191261829 | 2.493296773 | 2.342279301 |
| T204 | 0 | 0.011294701 | 0.00564735 |
| T205 | 0.233810854 | 0.285431409 | 0.259621131 |
| T206 | 10.01959464 | 20.5373915 | 15.27849307 |
| T207 | 10.49108906 | 2.516715376 | 6.503902216 |
| T208 | 0.014055027 | 0.015119552 | 0.01458729 |
| T209 | 0.105501842 | 0.240087981 | 0.172794911 |
| T210 | 0.032830508 | 0.505926007 | 0.269378258 |
| T211 | 0.015703077 | 0.01973562 | 0.017719349 |
| T212 | 0.179927375 | 1.109960579 | 0.644943977 |
| T213 | 0.006266764 | 0.133827266 | 0.070047015 |
| T214 | 0.006517259 | 0.013106004 | 0.009811631 |
| T215 | 0.146228914 | 0.277931794 | 0.212080354 |
| T216 | 0.006296066 | 0.028687916 | 0.017491991 |
| T217 | 0.907911803 | 2.643460991 | 1.775686397 |
| T218 | 2.9437143 | 5.212954747 | 4.078334524 |
| T219 | 0.037937883 | 0.022688472 | 0.030313177 |
| T220 | 0.033379625 | 0.022514795 | 0.02794721 |
| T221 | 0.003519048 | 0.007950841 | 0.005734944 |
| T222 | 0.757884765 | 1.860898065 | 1.309391415 |
| T223 | 0.535361475 | 0.325504003 | 0.430432739 |
| T224 | 0.025458036 | 0.016730225 | 0.021094131 |
| T225 | 0.002839054 | 0.004916328 | 0.003877691 |
| T226 | 16.20817491 | 27.72871483 | 21.96844487 |
| T227 | 0.770057493 | 2.629782447 | 1.69991997 |
| T228 | 0.00240797 | 0.01606107 | 0.00923452 |
| T229 | 1.40161354 | 0.661539075 | 1.031576307 |
| T230 | 0.267552148 | 0.430900621 | 0.349226384 |
| T231 | 0.173200035 | 0.144603306 | 0.158901671 |
| T232 | 0.005043161 | 0.238661255 | 0.121852208 |
| T233 | 0.022265595 | 0.352003618 | 0.187134607 |
| T234 | 0.005625343 | 0.007931472 | 0.006778408 |
| T235 | 0.006434711 | 0.004711376 | 0.005573043 |
| T236 | 0.027011828 | 0.027153548 | 0.027082688 |
| T237 | 0.051088067 | 0.180190144 | 0.115639106 |
| T238 | 0.726895119 | 0.846852138 | 0.786873629 |
| T239 | 0.011724198 | 0.007706909 | 0.009715553 |
| T240 | 0.036431977 | 0.035804065 | 0.036118021 |
| T241 | 0.159611537 | 0.151589784 | 0.15560066 |
| T242 | 0.036514208 | 0.029126602 | 0.032820405 |
| T243 | 0.33124651 | 0.262573477 | 0.296909993 |
| T244 | 0.314118169 | 0.242253672 | 0.27818592 |
| T245 | 0.000535084 | 0.00080377 | 0.000669427 |
| T246 | 0.034292201 | 0.032114913 | 0.033203557 |
| T247 | 0.040617978 | 0.041189685 | 0.040903831 |
| T248 | 0.027422751 | 0.025846776 | 0.026634763 |
| T249 | 0.037000262 | 0.034480881 | 0.035740572 |
| T250 | 0.008955699 | 0.007277112 | 0.008116406 |
| T251 | 0.021146526 | 0.021287133 | 0.02121683 |
| T252 | 0.008023951 | 0.008154996 | 0.008089474 |
| T253 | 0.001335042 | 0.007006351 | 0.004170697 |
| T254 | 0.006741377 | 0.003732266 | 0.005236821 |
| T255 | 0.03876 | 0.034421093 | 0.036590546 |
| T256 | 0.015885443 | 0.017778831 | 0.016832137 |
| T257 | 0.019807822 | 0.022322647 | 0.021065234 |
| T258 | 0.012899045 | 0.010518351 | 0.011708698 |
| T259 | 0.008915579 | 0.01477854 | 0.01184706 |
| T260 | 0.004318701 | 0.006281407 | 0.005300054 |
| T261 | 0.06122533 | 0.055329613 | 0.058277471 |
| T262 | 0.018765887 | 0.021226615 | 0.019996251 |
| T263 | 0 | 0.124736949 | 0.062368475 |
| T264 | 0.37260947 | 0.707568095 | 0.540088782 |
| T265 | 0.094206501 | 0.051860544 | 0.073033522 |
| T266 | 0.072877354 | 0.067538428 | 0.070207891 |
| T267 | 0.033354952 | 0.019052585 | 0.026203769 |
| T268 | 0.024802632 | 0.014804945 | 0.019803788 |
| T269 | 0.067459018 | 0.055016411 | 0.061237715 |
| T270 | 0.06809235 | 0.050819096 | 0.059455723 |
| T271 | 0.164880462 | 0.11959412 | 0.142237291 |
| T272 | 0.071431553 | 0.047870222 | 0.059650888 |
| T273 | 0.077991865 | 0.056745802 | 0.067368834 |
| T274 | 0.055053777 | 0.05056712 | 0.052810449 |
| T275 | 0.154850319 | 0.139368575 | 0.147109447 |
| T276 | 0.832091343 | 1.055925492 | 0.944008418 |
| T277 | 1.346311823 | 0.279060281 | 0.812686052 |
| T278 | 0.675855345 | 1.578755422 | 1.127305384 |
| T279 | 0.287799021 | 0.278415227 | 0.283107124 |
| T280 | 0 | 0.003364262 | 0.001682131 |
| T281 | 2.020285787 | 3.393977792 | 2.70713179 |
| T282 | 0.006872419 | 0.0053055 | 0.00608896 |
| T283 | 0.200774664 | 0.711796374 | 0.456285519 |
| T284 | 0.006719376 | 0.035994813 | 0.021357095 |
| T285 | 0.367210166 | 0.54378334 | 0.455496753 |
| T286 | 0.009887736 | 0.071853861 | 0.040870799 |
| T287 | 3.37826134 | 5.940996634 | 4.659628987 |
| T288 | 0.00391466 | 0.003868435 | 0.003891547 |
| T289 | 0.253261862 | 0.531294055 | 0.392277959 |
| T290 | 0.001402898 | 0.004620298 | 0.003011598 |
| T291 | 0.162313555 | 0.309318984 | 0.23581627 |
| T292 | 0.223956329 | 0.009903196 | 0.116929762 |
| T293 | 0.005478151 | 0.005873208 | 0.00567568 |
| T294 | 19.143748 | 13.08625984 | 16.11500392 |
| T295 | 0.005482782 | 0.005763006 | 0.005622894 |
| T296 | 0.005965896 | 0.006188101 | 0.006076998 |
| T297 | 0.007447529 | 0.006365626 | 0.006906577 |
| T298 | 0.012460149 | 0.017555817 | 0.015007983 |
| T299 | 0.003302043 | 0.004771675 | 0.004036859 |
| T300 | 0.018011643 | 0.021800809 | 0.019906226 |
| T301 | 0.004841794 | 0.003049668 | 0.003945731 |
| T302 | 0.010065131 | 0.0079551 | 0.009010116 |
| T303 | 0.079420711 | 0.102167847 | 0.090794279 |
| T304 | 0.005672773 | 0.006283756 | 0.005978265 |
| T305 | 0.008959035 | 0.00822423 | 0.008591632 |
| T306 | 0.00324412 | 0.003397307 | 0.003320713 |
| T307 | 0 | 0.000760395 | 0.000380197 |
| T308 | 0 | 0.000306316 | 0.000153158 |
| T309 | 0.026348946 | 0.025152645 | 0.025750796 |
| T310 | 0.017625169 | 0.011396516 | 0.014510843 |
| T311 | 0.01071688 | 0.016709435 | 0.013713157 |
| T312 | 0.002523946 | 0.002267291 | 0.002395618 |
| T313 | 0.024694048 | 0.027550739 | 0.026122394 |
| T314 | 2.586813625 | 0.443602421 | 1.515208023 |
| T315 | 0.006873356 | 0.006113684 | 0.00649352 |
| T316 | 0.013575842 | 0.011275596 | 0.012425719 |
| T317 | 0.013419291 | 0.019283924 | 0.016351607 |
| T318 | 0.091534749 | 0.070390124 | 0.080962436 |
| T319 | 0.001515274 | 0.001641314 | 0.001578294 |
| T320 | 0.029597474 | 0.006584579 | 0.018091027 |
| T321 | 0.02881218 | 0.014954095 | 0.021883137 |
| T322 | 0.00324924 | 0.002175203 | 0.002712222 |
| T323 | 0.011200933 | 0.00840435 | 0.009802642 |
| T324 | 0.011650938 | 0.015337288 | 0.013494113 |
| T325 | 0.031303931 | 0.030511269 | 0.0309076 |
| T326 | 0.004033541 | 0.004738483 | 0.004386012 |
| T327 | 0.049266626 | 0.047653632 | 0.048460129 |
| T328 | 0.016755544 | 0.01610141 | 0.016428477 |
| T329 | 0.002589144 | 0.002884704 | 0.002736924 |
| T330 | 0.03172599 | 0.020791608 | 0.026258799 |
| T331 | 0.008371482 | 0.002149963 | 0.005260723 |
| T332 | 0.013534604 | 0.02204663 | 0.017790617 |
| T333 | 0.001326066 | 0.002451691 | 0.001888879 |
| T334 | 0.184612411 | 0.039027944 | 0.111820177 |
| T335 | 0.011158906 | 0.007176862 | 0.009167884 |
| T336 | 0.003124878 | 0.002417052 | 0.002770965 |
| T337 | 0.001204268 | 0.001799689 | 0.001501978 |
| T338 | 0.015305096 | 0.017046746 | 0.016175921 |
| T339 | 0.013702384 | 0.014627903 | 0.014165143 |
| T340 | 0.015054401 | 0.014783305 | 0.014918853 |
| T341 | 0.012206038 | 0.013173016 | 0.012689527 |
| T342 | 0 | 2.375660531 | 1.187830266 |
| T343 | 0.011116155 | 0.007223818 | 0.009169986 |
| T344 | 1.335782932 | 0.484711373 | 0.910247152 |
| T345 | 0.005219083 | 0.009422748 | 0.007320915 |
| T346 | 0.033370749 | 0.033812434 | 0.033591591 |
| T347 | 0.003841278 | 0.00502938 | 0.004435329 |
| T348 | 0.012801824 | 0.010107664 | 0.011454744 |
| T349 | 0.039317135 | 0.053775543 | 0.046546339 |
| T350 | 0.009197184 | 0.006144033 | 0.007670609 |
| T351 | 0.004576938 | 0.006226121 | 0.00540153 |
| T352 | 0.010143333 | 0.011459144 | 0.010801239 |
| T353 | 0.00851518 | 0.008078506 | 0.008296843 |
| T354 | 0.000535776 | 0.002212389 | 0.001374083 |
| T355 | 0.608382154 | 0.194219614 | 0.401300884 |
| T356 | 0.211294233 | 0.119302569 | 0.165298401 |
| T357 | 0.051230119 | 0.01332288 | 0.032276499 |
| T358 | 0.009429237 | 0.015016883 | 0.01222306 |
| T359 | 0.019472246 | 0.011050732 | 0.015261489 |
| T360 | 0.013993777 | 0.016747053 | 0.015370415 |
| T361 | 0.034770837 | 0.035451701 | 0.035111269 |
| T362 | 0.008808042 | 0.009824296 | 0.009316169 |
| T363 | 0.00458521 | 0.00799832 | 0.006291765 |
| T364 | 0.016534847 | 0.00947037 | 0.013002608 |
| T365 | 0.037663347 | 0.04284374 | 0.040253543 |
| T366 | 0.011245997 | 0.010536684 | 0.010891341 |
| T367 | 0.02918338 | 0.02993615 | 0.029559765 |
| T368 | 0.874364749 | 0.222845582 | 0.548605165 |
| T369 | 0.029682084 | 0.019167226 | 0.024424655 |
| T370 | 0.001798804 | 0.004004111 | 0.002901457 |
| T371 | 0.003620808 | 0.003917593 | 0.003769201 |
| T372 | 0.020453296 | 0.016072139 | 0.018262717 |
| T373 | 0.02161254 | 0.02329866 | 0.0224556 |
| T374 | 0.008221549 | 0.010936339 | 0.009578944 |
| T375 | 0.01241575 | 0.013512428 | 0.012964089 |
| T376 | 0.010441634 | 0.013687627 | 0.01206463 |
| T377 | 0.014560774 | 0.006356091 | 0.010458432 |
| T378 | 0.004226563 | 0.005463372 | 0.004844967 |
| T379 | 0.003299132 | 0.003686258 | 0.003492695 |
| T380 | 0.168086736 | 0.099067861 | 0.133577299 |
| T381 | 0.008160734 | 0.011596633 | 0.009878684 |
| T382 | 0.021732455 | 0.024152896 | 0.022942675 |
| T383 | 0.032112893 | 0.033415224 | 0.032764058 |
| T384 | 0.040034922 | 0.039355181 | 0.039695051 |
| T385 | 0.036035117 | 0.031653765 | 0.033844441 |
| T386 | 0.025456756 | 0.024856866 | 0.025156811 |
| T387 | 0.004545212 | 0.00351845 | 0.004031831 |
| T388 | 0.067557978 | 0.069713799 | 0.068635888 |
| T389 | 0.02022314 | 0.011211131 | 0.015717136 |
| T390 | 0.013248594 | 0.015804126 | 0.01452636 |
| T391 | 0.01767545 | 0.014476079 | 0.016075764 |
| T392 | 0.017968775 | 0.01420684 | 0.016087807 |
| T393 | 0.011144237 | 0.011257112 | 0.011200675 |
| T394 | 0.020584774 | 0.019419751 | 0.020002263 |

### Example 11: Indel Activity of CasPhi.12 Effector Protein (SEQ ID NO: 2)

In a second indel activity analysis, combinations of the effector protein (SEQ ID NO: 2) and guide nucleic acids as set forth in TABLE 18 targeting the DMD gene were tested as described in Example 10 for their ability to produce indels in the DMD gene.

Indels were detected and analyzed as set forth in Example 10 and are provided in TABLE 19.

TABLE 18
CasPhi.12 Effector Protein (SEQ ID NO:
2) and Guide Nucleic Acid Combinations

| | | | | crRNA |
|---|---|---|---|---|
| | | Repeat Sequence | Spacer sequence | Sequence SEQ |
| Name | PAM | SEQ ID NO: | SEQ ID NO: | ID NO |
| T395_CasPhi.12 | NTTN | 740 | 506 | 1226 |
| T396_CasPhi.12 | NTTN | 740 | 507 | 1227 |
| T397_CasPhi.12 | NTTN | 740 | 508 | 1228 |
| T398_CasPhi.12 | NTTN | 740 | 509 | 1229 |
| T399_CasPhi.12 | NTTN | 740 | 510 | 1230 |
| T400_CasPhi.12 | NTTN | 740 | 511 | 1231 |
| T401_CasPhi.12 | NTTN | 740 | 512 | 1232 |
| T402_CasPhi.12 | NTTN | 740 | 513 | 1233 |
| T403_CasPhi.12 | NTTN | 740 | 514 | 1234 |
| T404_CasPhi.12 | NTTN | 740 | 515 | 1235 |
| T405_CasPhi.12 | NTTN | 740 | 516 | 1236 |
| T406_CasPhi.12 | NTTN | 740 | 517 | 1237 |
| T407_CasPhi.12 | NTTN | 740 | 518 | 1238 |
| T408_CasPhi.12 | NTTN | 740 | 519 | 1239 |
| T409_CasPhi.12 | NTTN | 740 | 520 | 1240 |
| T410_CasPhi.12 | NTTN | 740 | 521 | 1241 |
| T411_CasPhi.12 | NTTN | 740 | 522 | 1242 |
| T412_CasPhi.12 | NTTN | 740 | 523 | 1243 |
| T413_CasPhi.12 | NTTN | 740 | 524 | 1244 |
| T414_CasPhi.12 | NTTN | 740 | 525 | 1245 |
| T415_CasPhi.12 | NTTN | 740 | 526 | 1246 |
| T416_CasPhi.12 | NTTN | 740 | 527 | 1247 |
| T417_CasPhi.12 | NTTN | 740 | 528 | 1248 |
| T418_CasPhi.12 | NTTN | 740 | 529 | 1249 |
| T419_CasPhi.12 | NTTN | 740 | 530 | 1250 |
| T420_CasPhi.12 | NTTN | 740 | 531 | 1251 |
| T421_CasPhi.12 | NTTN | 740 | 532 | 1252 |
| T422_CasPhi.12 | NTTN | 740 | 533 | 1253 |
| T423_CasPhi.12 | NTTN | 740 | 534 | 1254 |
| T424_CasPhi.12 | NTTN | 740 | 535 | 1255 |
| T425_CasPhi.12 | NTTN | 740 | 536 | 1256 |
| T426_CasPhi.12 | NTTN | 740 | 537 | 1257 |
| T427_CasPhi.12 | NTTN | 740 | 538 | 1258 |
| T428_CasPhi.12 | NTTN | 740 | 539 | 1259 |
| T429_CasPhi.12 | NTTN | 740 | 540 | 1260 |
| T430_CasPhi.12 | NTTN | 740 | 541 | 1261 |
| T431_CasPhi.12 | NTTN | 740 | 542 | 1262 |
| T432_CasPhi.12 | NTTN | 740 | 543 | 1263 |
| T433_CasPhi.12 | NTTN | 740 | 544 | 1264 |
| T434_CasPhi.12 | NTTN | 740 | 545 | 1265 |
| T435_CasPhi.12 | NTTN | 740 | 546 | 1266 |
| T436_CasPhi.12 | NTTN | 740 | 547 | 1267 |
| T437_CasPhi.12 | NTTN | 740 | 548 | 1268 |
| T438_CasPhi.12 | NTTN | 740 | 549 | 1269 |
| T439_CasPhi.12 | NTTN | 740 | 550 | 1270 |
| T440_CasPhi.12 | NTTN | 740 | 551 | 1271 |
| T441_CasPhi.12 | NTTN | 740 | 552 | 1272 |
| T442_CasPhi.12 | NTTN | 740 | 553 | 1273 |
| T443_CasPhi.12 | NTTN | 740 | 554 | 1274 |
| T444_CasPhi.12 | NTTN | 740 | 555 | 1275 |
| T445_CasPhi.12 | NTTN | 740 | 556 | 1276 |
| T446_CasPhi.12 | NTTN | 740 | 557 | 1277 |
| T447_CasPhi.12 | NTTN | 740 | 558 | 1278 |
| T448_CasPhi.12 | NTTN | 740 | 559 | 1279 |
| T449_CasPhi.12 | NTTN | 740 | 560 | 1280 |
| T450_CasPhi.12 | NTTN | 740 | 561 | 1281 |
| T451_CasPhi.12 | NTTN | 740 | 562 | 1282 |
| T452_CasPhi.12 | NTTN | 740 | 563 | 1283 |
| T453_CasPhi.12 | NTTN | 740 | 564 | 1284 |
| T454_CasPhi.12 | NTTN | 740 | 565 | 1285 |
| T455_CasPhi.12 | NTTN | 740 | 566 | 1286 |
| T456_CasPhi.12 | NTTN | 740 | 567 | 1287 |
| T457_CasPhi.12 | NTTN | 740 | 568 | 1288 |
| T458_CasPhi.12 | NTTN | 740 | 569 | 1289 |
| T459_CasPhi.12 | NTTN | 740 | 570 | 1290 |
| T460_CasPhi.12 | NTTN | 740 | 571 | 1291 |
| T461_CasPhi.12 | NTTN | 740 | 572 | 1292 |
| T462_CasPhi.12 | NTTN | 740 | 573 | 1293 |
| T463_CasPhi.12 | NTTN | 740 | 574 | 1294 |
| T464_CasPhi.12 | NTTN | 740 | 575 | 1295 |
| T465_CasPhi.12 | NTTN | 740 | 576 | 1296 |
| T466_CasPhi.12 | NTTN | 740 | 577 | 1297 |
| T467_CasPhi.12 | NTTN | 740 | 578 | 1298 |
| T468_CasPhi.12 | NTTN | 740 | 579 | 1299 |
| T469_CasPhi.12 | NTTN | 740 | 580 | 1300 |
| T470_CasPhi.12 | NTTN | 740 | 581 | 1301 |
| T471_CasPhi.12 | NTTN | 740 | 582 | 1302 |
| T472_CasPhi.12 | NTTN | 740 | 583 | 1303 |
| T473_CasPhi.12 | NTTN | 740 | 584 | 1304 |
| T474_CasPhi.12 | NTTN | 740 | 585 | 1305 |
| T475_CasPhi.12 | NTTN | 740 | 586 | 1306 |
| T476_CasPhi.12 | NTTN | 740 | 587 | 1307 |
| T477_CasPhi.12 | NTTN | 740 | 588 | 1308 |
| T478_CasPhi.12 | NTTN | 740 | 589 | 1309 |
| T479_CasPhi.12 | NTTN | 740 | 590 | 1310 |
| T480_CasPhi.12 | NTTN | 740 | 591 | 1311 |
| T481_CasPhi.12 | NTTN | 740 | 592 | 1312 |
| T482_CasPhi.12 | NTTN | 740 | 593 | 1313 |
| T483_CasPhi.12 | NTTN | 740 | 594 | 1314 |
| T484_CasPhi.12 | NTTN | 740 | 595 | 1315 |
| T485_CasPhi.12 | NTTN | 740 | 596 | 1316 |
| T486_CasPhi.12 | NTTN | 740 | 597 | 1317 |
| T487_CasPhi.12 | NTTN | 740 | 598 | 1318 |
| T488_CasPhi.12 | NTTN | 740 | 599 | 1319 |
| T489_CasPhi.12 | NTTN | 740 | 600 | 1320 |
| T490_CasPhi.12 | NTTN | 740 | 601 | 1321 |
| T491_CasPhi.12 | NTTN | 740 | 602 | 1322 |
| T492_CasPhi.12 | NTTN | 740 | 603 | 1323 |
| T493_CasPhi.12 | NTTN | 740 | 604 | 1324 |
| T494_CasPhi.12 | NTTN | 740 | 605 | 1325 |
| T495_CasPhi.12 | NTTN | 740 | 606 | 1326 |
| T496_CasPhi.12 | NTTN | 740 | 607 | 1327 |
| T497_CasPhi.12 | NTTN | 740 | 608 | 1328 |
| T498_CasPhi.12 | NTTN | 740 | 609 | 1329 |
| T499_CasPhi.12 | NTTN | 740 | 610 | 1330 |
| T500_CasPhi.12 | NTTN | 740 | 611 | 1331 |
| T501_CasPhi.12 | NTTN | 740 | 612 | 1332 |
| T502_CasPhi.12 | NTTN | 740 | 613 | 1333 |
| T503_CasPhi.12 | NTTN | 740 | 614 | 1334 |
| T504_CasPhi.12 | NTTN | 740 | 615 | 1335 |
| T505_CasPhi.12 | NTTN | 740 | 616 | 1336 |
| T506_CasPhi.12 | NTTN | 740 | 617 | 1337 |
| T507_CasPhi.12 | NTTN | 740 | 618 | 1338 |
| T508_CasPhi.12 | NTTN | 740 | 619 | 1339 |
| T509_CasPhi.12 | NTTN | 740 | 620 | 1340 |

TABLE 19
Indel activity of Effector Protein (SEQ ID NO: 2)
and Guide Nucleic Acid Combinations of TABLE 16

| Name | % indel rep 1 | % indel rep 2 |
|---|---|---|
| T395_CasPhi.12 | 17.52766 | 18.05646 |
| T396_CasPhi.12 | 20.69294 | 20.47271 |
| T397_CasPhi.12 | 1.421346 | 1.402211 |
| T398_CasPhi.12 | 0.058726 | 0.102264 |
| T399_CasPhi.12 | 0.184822 | 0.263499 |
| T400_CasPhi.12 | 2.887844 | 3.063028 |
| T401_CasPhi.12 | 1.364796 | 1.189648 |
| T402_CasPhi.12 | 31.64254 | 31.3915 |
| T403_CasPhi.12 | 0.014618 | 0.04073 |
| T404_CasPhi.12 | 0.208789 | 0.256838 |
| T405_CasPhi.12 | 0.153803 | 0.186356 |
| T406_CasPhi.12 | 0.564436 | 0.754748 |
| T407_CasPhi.12 | 0.48962 | 0.57684 |
| T408_CasPhi.12 | 13.46827 | 13.62409 |
| T409_CasPhi.12 | 38.34436 | 37.52483 |
| T410_CasPhi.12 | 0.218955 | 0.289957 |
| T411_CasPhi.12 | 0 | 0 |
| T412_CasPhi.12 | 0.122127 | 0.103297 |
| T413_CasPhi.12 | 0.511509 | 0.417965 |
| T414_CasPhi.12 | 0.020614 | 0.023766 |
| T415_CasPhi.12 | 0 | 0.005127 |
| T416_CasPhi.12 | 0.013253 | 0.013313 |
| T417_CasPhi.12 | 0.277452 | 0.255591 |
| T418_CasPhi.12 | 0 | 0 |
| T419_CasPhi.12 | 0.772947 | 0.796412 |
| T420_CasPhi.12 | 16.63542 | 13.94191 |
| T421_CasPhi.12 | 1.132421 | 1.166341 |
| T422_CasPhi.12 | 6.865831 | 6.848526 |
| T423_CasPhi.12 | 0.017013 | 0.019704 |
| T424_CasPhi.12 | 0.222737 | 0.226566 |
| T425_CasPhi.12 | 3.964561 | 4.059336 |
| T426_CasPhi.12 | 27.75346 | 27.6211 |
| T427_CasPhi.12 | 1.450723 | 1.557439 |
| T428_CasPhi.12 | 25.46039 | 24.36063 |
| T429_CasPhi.12 | 3.510436 | 3.766334 |
| T430_CasPhi.12 | 0.351317 | 0.29092 |
| T431_CasPhi.12 | 0.174252 | 0.139609 |
| T432_CasPhi.12 | 1.462589 | 1.655934 |
| T433_CasPhi.12 | 0.003888 | 0 |
| T434_CasPhi.12 | 0.248949 | 0.122399 |
| T435_CasPhi.12 | 12.99535 | 14.22222 |
| T436_CasPhi.12 | 18.31179 | 18.51395 |
| T437_CasPhi.12 | 21.52709 | 22.7154 |
| T438_CasPhi.12 | 61.87014 | 61.99009 |
| T439_CasPhi.12 | 25.8304 | 25.76298 |
| T440_CasPhi.12 | 0.307245 | 0.297012 |
| T441_CasPhi.12 | 0.053861 | 0.073904 |
| T442_CasPhi.12 | 12.49123 | 12.39396 |
| T443_CasPhi.12 | 0 | 0.033613 |
| T444_CasPhi.12 | 0.003797 | 0 |
| T445_CasPhi.12 | 10.02611 | 9.427347 |
| T446_CasPhi.12 | 0.006705 | 0 |
| T447_CasPhi.12 | 0.05443 | 0.031579 |
| T448_CasPhi.12 | 0 | 0 |
| T449_CasPhi.12 | 53.06464 | 53.77097 |
| T450_CasPhi.12 | 0.013639 | 0 |
| T451_CasPhi.12 | 0.009426 | 0.004321 |
| T452_CasPhi.12 | 16.56522 | 15.5592 |
| T453_CasPhi.12 | 4.554865 | 4.725493 |
| T454_CasPhi.12 | 0.166081 | 0.156895 |
| T455_CasPhi.12 | 1.422663 | 1.502374 |
| T456_CasPhi.12 | 0.063281 | 0.142146 |
| T457_CasPhi.12 | 0.472927 | 0.477425 |
| T458_CasPhi.12 | 2.153159 | 2.039721 |
| T459_CasPhi.12 | 2.643135 | 2.439246 |
| T460_CasPhi.12 | 0.007829 | 0.016814 |
| T461_CasPhi.12 | 1.017736 | 1.250167 |
| T462_CasPhi.12 | 1.847971 | 1.868514 |
| T463_CasPhi.12 | 12.06595 | 12.18331 |
| T464_CasPhi.12 | 0.004395 | 0.008222 |
| T465_CasPhi.12 | 1.425323 | 1.531223 |
| T466_CasPhi.12 | 0.012351 | 0.013246 |
| T467_CasPhi.12 | 0 | 0 |
| T468_CasPhi.12 | 0 | 0.00687 |
| T469_CasPhi.12 | 4.053013 | 4.167431 |
| T470_CasPhi.12 | 0.013292 | 0.014837 |
| T471_CasPhi.12 | 0.159196 | 0.23281 |
| T472_CasPhi.12 | 0.004251 | 0.007702 |
| T473_CasPhi.12 | 2.490927 | 2.689089 |
| T474_CasPhi.12 | 1.16113 | 1.021795 |
| T475_CasPhi.12 | 6.935189 | 6.638079 |
| T476_CasPhi.12 | 0.030823 | 0.010938 |
| T477_CasPhi.12 | 4.658894 | 4.983591 |
| T478_CasPhi.12 | 0.033413 | 0.0324 |
| T479_CasPhi.12 | 0.009815 | 0.002863 |
| T480_CasPhi.12 | 0 | 0 |
| T481_CasPhi.12 | 0.016962 | 0.014264 |
| T482_CasPhi.12 | 0.060386 | 0.055398 |
| T483_CasPhi.12 | 0 | 0.00461 |
| T484_CasPhi.12 | 0.003672 | 0.014181 |
| T485_CasPhi.12 | 10.09865 | 9.737719 |
| T486_CasPhi.12 | 0.369519 | 0.388528 |
| T487_CasPhi.12 | 0.031491 | 0.031243 |
| T488_CasPhi.12 | 0.01372 | 0.017649 |
| T489_CasPhi.12 | 2.571453 | 2.673749 |
| T490_CasPhi.12 | 0.007854 | 0.004263 |
| T491_CasPhi.12 | 9.323782 | 6.687023 |
| T492_CasPhi.12 | 1.107283 | 0.940713 |
| T493_CasPhi.12 | 0.025221 | 0 |
| T494_CasPhi.12 | 3.065554 | 2.006031 |
| T495_CasPhi.12 | 0.010983 | 0 |
| T496_CasPhi.12 | 0 | 0 |
| T497_CasPhi.12 | 2.121116 | 1.41173 |
| T498_CasPhi.12 | 0 | 0 |
| T499_CasPhi.12 | 0 | 0 |
| T500_CasPhi.12 | 0 | 0 |
| T501_CasPhi.12 | 17.29694 | 17.53864 |
| T502_CasPhi.12 | 1.882443 | 1.163703 |
| T503_CasPhi.12 | 3.136642 | 3.935396 |
| T504_CasPhi.12 | 0.172078 | 0.069614 |
| T505_CasPhi.12 | 0.142383 | 0.121024 |
| T506_CasPhi.12 | 6.453149 | 4.342621 |
| T507_CasPhi.12 | 3.642987 | 2.381719 |
| T508_CasPhi.12 | 0.058038 | 0.01638 |
| T509_CasPhi.12 | 0.328994 | 0.183352 |

### Example 13: Indel Activity of CasPhi.12 Effector Protein (SEQ ID NO: 2) in Cardiomyocytes: Lipofection, Viability and Expression of eGFP (Plasmid and mRNA Delivery) and Indel Activity in iPSC-Derived Cardiomyocytes

### Example 13.1: Lipofection, Viability and Expression of eGFP (Plasmid and mRNA Delivery) in iPSC-Derived Cardiomyocytes

This experiment assessed cell viability and eGFP expression 48 hours of iPSC derived cardiomyocytes post lipofection.

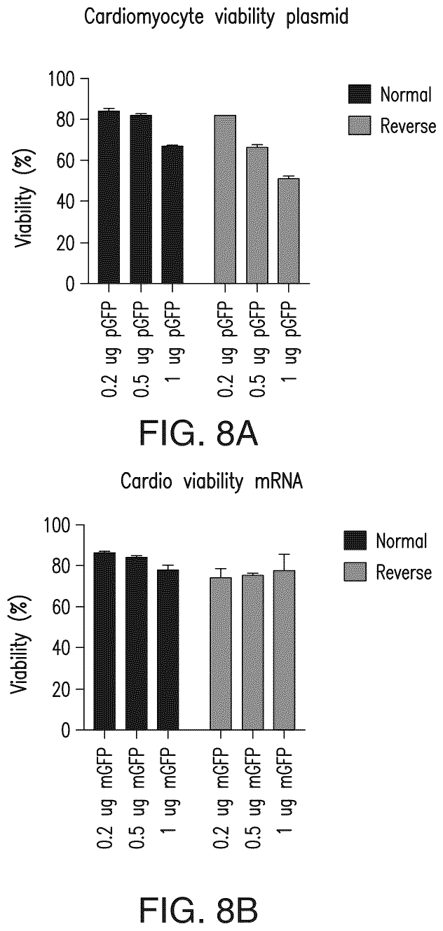

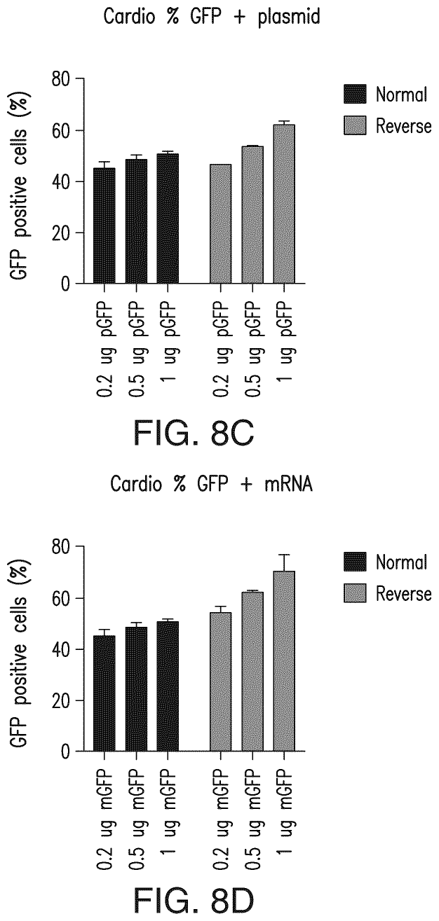

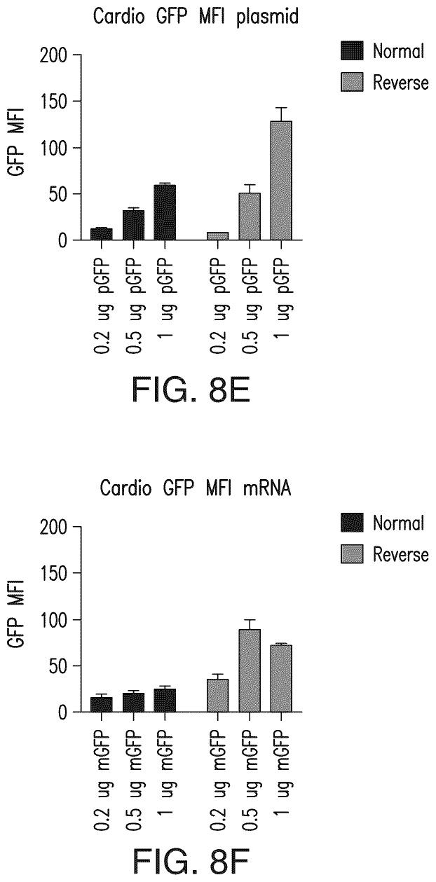

Lipofection of iPSC derived cardiomyocytes: Briefly, iPSC derived cardiomyocytes were purchased and cultured according to Takara Bio Europe AB, Cellartis® Cardiomyocytes User Manual. Cat. No. Y10075, pp. 1-6 (2018). Plasmid or mRNA encoding GFP were delivered by lipofection as described in ThermoFisher Scientific, Lipofectamine™ Stem Transfection Reagent. Pub. No. MAN0017080, pp. 1-2 (2017) and in TAN et al., “Non-viral vector based gene transfection with human induced pluripotent stem cells derived cardiomyocytes,” Sci. Reports, 9:14404 (2019) (modifying ThermoFischer Scientific, 2017 in terms of kit and lipid to DNA ratio). Results are shown in FIGS. 8A and 8B and demonstrate successful lipofection of iPSC derived cardiomyocytes. Normal and reverse lipofection refer to cells attached and cells floating, respectively.

Cardiomyocytes GFP mRNA and plasmid expression after 48 h: GFP positivity of mRNA and plasmid delivered cardiomyocytes was measured 48 hours after lipofection by flow cytometry to establish the incidence of GFP expression. Results can be seen in FIGS. 8C and 8D. Mean fluorescence intensity (MFI) was measured 48 hours after lipofection by flow cytometry to establish the level of GFP expression. Results can be seen in FIGS. 8E and 8F.

### Example 13.2: Indel Activity of Effector Protein in Cardiomyocytes Compared to a GFP Control

Plasmids expressing the effector protein/guide nucleic acid combinations and eGFP targeting the DMD gene were delivered by lipofection to iPSC derived cardiomyocytes as set forth in Example 13.1. The full sequence of the effector protein used in the present example is:

(SEQ ID NO: 1468)
PKKKRKVGIHGVPAAMIKPTVSQFLTPGFKLIRNHSRTAGLKLKNEGEE
ACKKFVRENEIPKDECPNFQGGPAIANIIAKSREFTEWEIYQSSLAIQE
VIFTLPKDKLPEPILKEEWRAQWLSEHGLDTVPYKEAAGLNLIIKNAVN
TYKGVQVKVDNKNKNNLAKINRKNEIAKLNGEQEISFEEIKAFDDKGYL
LQKPSPNKSIYCYQSVSPKPFITSKYHNVNLPEEYIGYYRKSNEPIVSP
YQFDRLRIPIGEPGYVPKWQYTFLSKKENKRRKLSKRIKNVSPILGIIC
IKKDWCVFDMRGLLRTNHWKKYHKPTDSINDLFDYFTGDPVIDTKANVV
RFRYKMENGIVNYKPVREKKGKELLENICDQNGSCKLATVDVGQNNPVA
IGLFELKKVNGELTKTLISRHPTPIDFCNKITAYRERYDKLESSIKLDA
IKQLTSEQKIEVDNYNNNFTPQNTKQIVCSKLNINPNDLPWDKMISGTH
FISEKAQVSNKSEIYFTSTDKGKTKDVMKSDYKWFQDYKPKLSKEVRDA
LSDIEWRLRRESLEFNKLSKSREQDARQLANWISSMCDVIGIENLVKKN
NFFGGSGKREPGWDNFYKPKKENRWWINAIHKALTELSQNKGKRVILLP
AMRTSITCPKCKYCDSKNRNGEKFNCLKCGIELNADIDVATENLATVAI
TAQSMPKPTCERSGDAKKPVRARKAKAPEFHDKLAPSYTVVLREAVKRP
AATKKAGQAKKKK,

wherein PKKKRKVGIHGVPAA (SEQ ID NO: 21) and KRPAATKKAGQAKKKK (SEQ ID NO: 22) are NLS′. Effector protein and guide nucleic acid combinations were delivered on the same vector and are as set forth in TABLE 20 below. Tested guide nucleic acids each used a sequence of 5′-gAUUGCUCCUUACGAGGAGAC-3′ (SEQ ID NO: 741), where g was added as the transcription start site and the repeat sequence is 5′-AUUGCUCCUUACGAGGAGAC-3′ (SEQ ID NO: 740), with a spacer sequence of TABLE 20 linked to the 3′ end.

TABLE 20
Effector Protein (SEQ ID NO: 2) and
Guide Nucleic Acid Combinations

| | Effector | | Repeat | Spacer |
|---|---|---|---|---|
| | protein SEQ | | Sequence | Sequence |
| Plasmid | ID NO: | PAM | SEQ ID NO: | SEQ ID NO: |
| PL16071 | 2 (Full | TTTA | 740 | 337 |
| | Sequence | | | |
| | SEQ ID NO: | | | |
| | 1468) | | | |
| PL16271 | 2 (Full | TTTG | 740 | 537 |
| | Sequence | | | |
| | SEQ ID NO: | | | |
| | 1469) | | | |

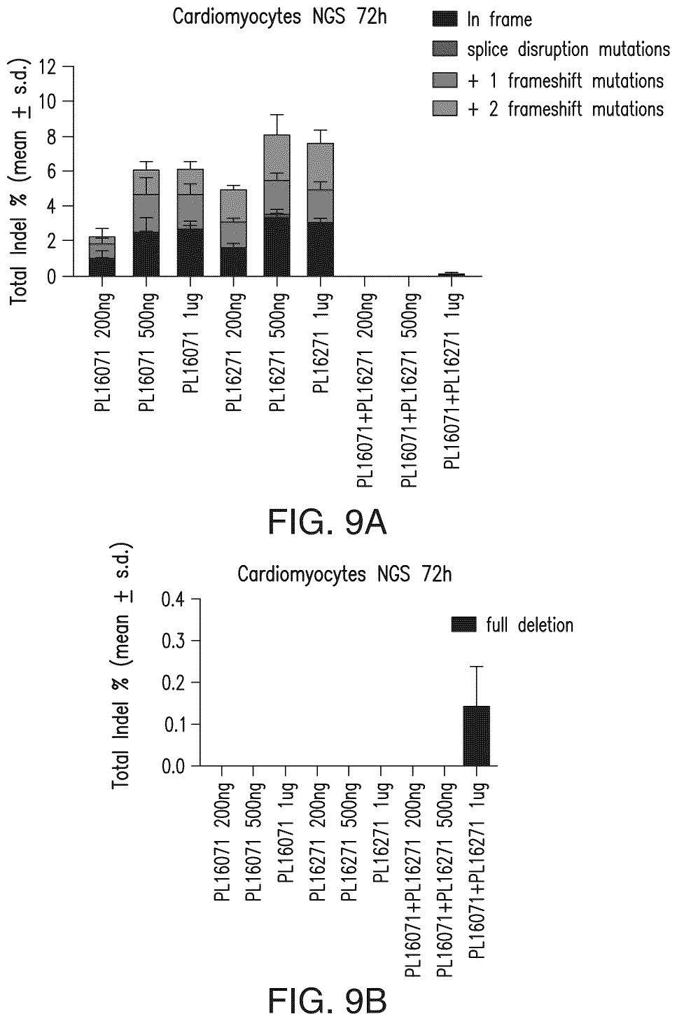

Single and Dual cutting was assessed by delivery of one or two guides, respectively. GFP expression and indel activity was assessed 72 hours post lipofection. Results indicate indel activity. Prediction of indels were made based on NGS data as described in Example 3 and can be seen in FIGS. 9A and 9B. FIGS. 9A and 9B demonstrate that effector protein (SEQ ID NO: 2) and guide nucleic acid combinations can be predicted to effect in frame, +1 frameshift, +2 framcshift mutations, and full sequence deletion/dual cutting. Splice disruptions mutations and 1+ frameshift mutations are predicted to be the most helpful for DMD gene editing. Dual cutting can be seen in FIG. 9B.

### Example 14: Indel Activity of CasPhi.12 Effector Protein (SEQ ID NO: 2) in Myoblasts: Lipofection, Viability and Expression of eGFP (Plasmid and mRNA Delivery) and Indel Activity in iPSC-Derived Myoblasts

### Example 14.1: Lipofection, Viability and Expression of eGFP (Plasmid and mRNA Delivery) in iPSC-Derived Myoblasts

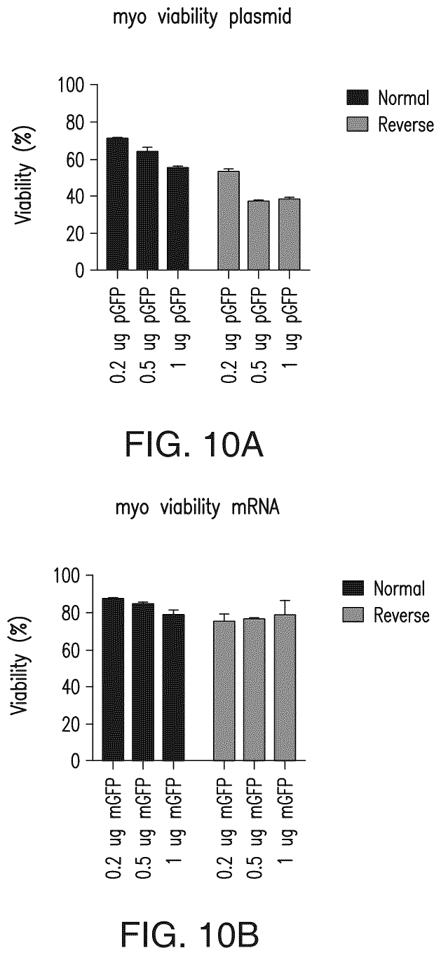

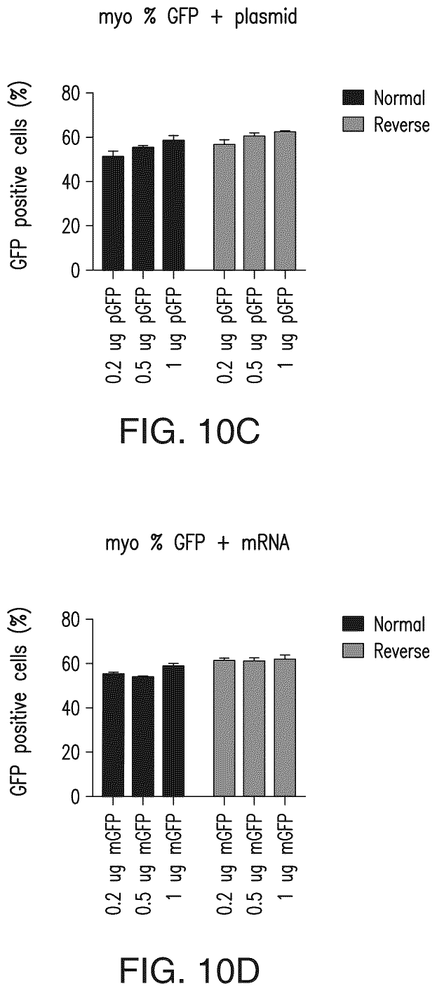

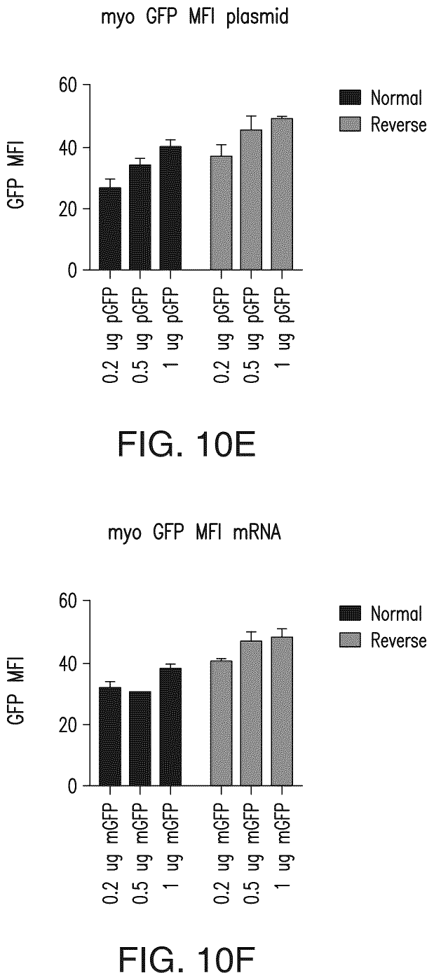

Lipofection of iPSC derived myoblasts: iPSC derived myoblasts were purchased and cultured according to Life Technologies Corporation. HSkM-S. Cat. No. A12555, pp. 1-2 (2010). Plasmid or mRNA encoding GFP were delivered by lipofection as described in ThermoFisher Scientific. Lipofectamine™ Stem Transfection Reagent. Pub. No. MAN0017080, pp. 1-2 (2017) and in TAN et al., “Non-viral vector based gene transfection with human induced pluripotent stem cells derived cardiomyocytes.” Sci. Reports, 9:14404 (2019) as described for cardiomyocytes above. Results are shown in FIGS. 10A and 10B and demonstrate successful lipofection of iPSC derived myoblasts.

Myoblasts GFP mRNA and plasmid expression after 48 h: GFP positivity of mRNA and plasmid delivered myoblasts was measured 48 hours after lipofection by flow cytometry to establish the incidence of GFP expression. Results can be seen in FIGS. 10C and 10D. Mean fluorescence intensity (MFI) was measured 48 hours after lipofection by flow cytometry to establish the level of GFP expression. Results can be seen in FIGS. 10E and 10F.

### Example 14.2: Indel Activity of Effector Protein in Myoblasts Compared to GFP Control

Single and Dual cutting is assessed by delivery of one or two guides, respectively. GFP expression and indel activity was assessed 72 hours post lipofection. Results indicate indel activity.

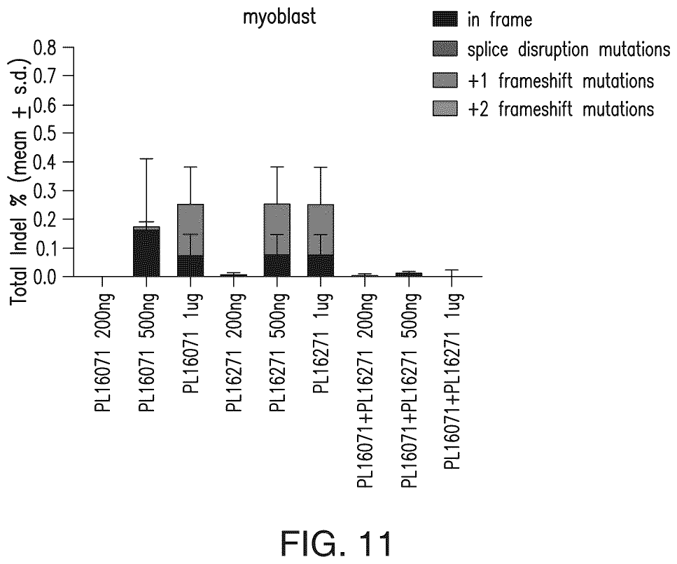

Prediction of indels were made based on NGS data as described in Example 3 and can be seen in FIG. 11. FIG. 11 demonstrates that effector protein and guide nucleic acid combinations can be predicted to effect in frame and +1 frameshift mutations. Some activity can also be seen in two guide systems.

### Example 15: Indel Activity of CasPhi.12 Effector Protein Variant and Indel Activity in iPSC-Derived Myoblasts

iPSC derived myoblasts were purchased, cultured and lipofected similar to the methods described in Examples 10, 13 and 14 with an RNP complex having an CasPhi.12 variant having an L26R substitution (relative to SEQ ID NO: 2) with a guide nucleic acid (crRNA) having sequences as set forth in TABLE 21. Indel activity was assessed as described in Example 14. As much as 4% indel was observed.

TABLE 21
Effector protein and Guide Combinations

| | Repeat Sequence | Spacer Sequence | crRNA SEQ |
|---|---|---|---|
| Effector Protein | SEQ ID NO: | SEQ ID NO: | ID NO: |
| CasPhi.12 L26R | 742 | 183 | 1341 |
| Variant | | | |
| CasPhi.12 L26R | 742 | 182 | 1342 |
| Variant | | | |

### Example 16. CasPhi.12 Mediated DMD Exon Deletion in HEK293T Cells

Guide pairs targeting DMD were screened in HEK293T cells for the identification and selection of guides for exon deletion therapeutic strategies. Plasmids co-expressing CasPhi.12 and gRNA (1 plasmid/target) were tested in pairs for dual cut deletions of certain DMD locus exons (44, 45, 50, 51, or 53). Plasmid pairs were co-transfected in HEK293T cells via lipofection. Cells were incubated for 72 hours before being harvested for DNA, PCR amplified and sequenced via NGS. The sequencing data were then analyzed using CRISPRESSO to detect/quantify % indel and exon deletions.

Guide nucleic acids used a crRNA repeat represented by the sequence: AUUGCUCCUUACGAGGAGAC (SEQ ID NO: 740). Spacer sequences were located 3′ of the crRNA repeat sequence. Spacer sequences are set forth in TABLE 22.

The full sequence of the polypeptide used in this experiment is:

(SEQ ID NO: 1469)
MDYKDHDGDYKDHDIDYKDDDDKMAPKKKRKVGIHGVPAAMIKPTVSQF
LTPGFKLIRNHSRTAGLKLKNEGEEACKKFVRENEIPKDECPNFQGGPA
IANIIAKSREFTEWEIYQSSLAIQEVIFTLPKDKLPEPILKEEWRAQWL
SEHGLDTVPYKEAAGLNLIIKNAVNTYKGVQVKVDNKNKNNLAKINRKN
EIAKLNGEQEISFEEIKAFDDKGYLLQKPSPNKSIYCYQSVSPKPFITS
KYHNVNLPEEYIGYYRKSNEPIVSPYQFDRLRIPIGEPGYVPKWQYTFL
SKKENKRRKLSKRIKNVSPILGIICIKKDWCVFDMRGLLRTNHWKKYHK
PTDSINDLFDYFTGDPVIDTKANVVRFRYKMENGIVNYKPVREKKGKEL
LENICDQNGSCKLATVDVGQNNPVAIGLFELKKVNGELTKTLISRHPTP
IDFCNKITAYRERYDKLESSIKLDAIKQLTSEQKIEVDNYNNNFTPQNT
KQIVCSKLNINPNDLPWDKMISGTHFISEKAQVSNKSEIYFTSTDKGKT
KDVMKSDYKWFQDYKPKLSKEVRDALSDIEWRLRRESLEFNKLSKSREQ
DARQLANWISSMCDVIGIENLVKKNNFFGGSGKREPGWDNFYKPKKENR
WWINAIHKALTELSQNKGKRVILLPAMRTSITCPKCKYCDSKNRNGEKF
NCLKCGIELNADIDVATENLATVAITAQSMPKPTCERSGDAKKPVRARK
AKAPEFHDKLAPSYTVVLREAVKRPAATKKAGQAKKKKEFGSGEGRGSL
LTCGDVEENPGPMAKPLSQEESTLIERATATINSIPISEDYSVASAALS
SDGRIFTGVNVYHFTGGPCAELVVLGTAAAAAAGNLTCIVAIGNENRGI
LSPCGRCRQVLLDLHPGIKAIVKDSDGQPTAVGIRELLPSGYVWEG*,

wherein MDYKDHDGDYKDHDIDYKDDDDK (SEQ ID NO: 23) is a FLAG tag; MAPKKKRKV (SEQ ID NO: 24) is a nuclear localization signal (NLS) (SV40); GIHGVPAA (SEQ ID NO: 25) is a linker; KRPAATKKAGQAKKKK (SEQ ID NO: 22) is nucleoplasmin NLS; EFGSGEGRGSLLTCGDVEENPGP (SEQ ID NO: 26) is a linker+T2A sequence (self cleaving peptide); MAKPLSOEESTLIERATATINSIPISEDYSVASAALSSDGRIFTGVNVYHETGGPCAEL VVLGTAAAAAAGNLTCIVAIGNENRGILSPCGRCROVLLDLHPGIKAIVKDSDGOPT AVGIRELLPSGYVWEG (SEQ ID NO: 27) confers blasticidin resistance; and the remainder of the sequence represents the CasPhi.12 effector protein (SEQ ID NO: 2).

Total indel of different exon deletions are provided in TABLE 22 (obtained from NGS data). Results demonstrated that combinations of nuclease and gRNA pairs (dual cut) can be used to delete an entire exon (44, 45, 50, 51, or 53), thereby resulting in skipping of that exon during translation and protein production.

TABLE 22
Indel Activity and Exon Deletion

| | Repeat Sequence | Spacer-1 | Spacer-2 | % indel | % indel | Full Region | Full Region | |
|---|---|---|---|---|---|---|---|---|
| No. | SEQ ID NO: | SEQ ID NO: | SEQ ID NO: | rep1 | rep2 | Deletion Rep1 | Deletion Rep2 | Exon |
| 1 | 740 | 119 | 166 | +++ | +++ | *** | ND | 44 |
| 2 | 740 | 119 | 150 | +++ | +++ | ND | *** | 44 |
| 3 | 740 | 181 | 249 | + | + | * | * | 45 |
| 4 | 740 | 181 | 236 | + | + | * | * | 45 |
| 5 | 740 | 181 | 244 | + | + | * | * | 45 |
| 6 | 740 | 181 | 220 | + | + | * | * | 45 |
| 7 | 740 | 181 | 221 | +++ | +++ | * | ** | 45 |
| 8 | 740 | 182 | 249 | ++ | +++ | * | * | 45 |
| 9 | 740 | 182 | 236 | +++ | +++ | ** | *** | 45 |
| 10 | 740 | 182 | 244 | +++ | +++ | ** | ** | 45 |
| 11 | 740 | 182 | 220 | +++ | +++ | ** | ** | 45 |
| 12 | 740 | 182 | 221 | + | + | * | * | 45 |
| 13 | 740 | 183 | 249 | +++ | +++ | * | ** | 45 |
| 14 | 740 | 183 | 236 | +++ | +++ | *** | *** | 45 |
| 15 | 740 | 183 | 244 | +++ | +++ | ** | ** | 45 |
| 16 | 740 | 183 | 220 | +++ | +++ | ** | * | 45 |
| 17 | 740 | 183 | 221 | +++ | +++ | * | * | 45 |
| 18 | 740 | 186 | 249 | +++ | +++ | * | * | 45 |
| 19 | 740 | 186 | 236 | +++ | +++ | ** | ** | 45 |
| 20 | 740 | 186 | 244 | +++ | +++ | * | * | 45 |
| 21 | 740 | 186 | 220 | +++ | +++ | ** | ** | 45 |
| 22 | 740 | 186 | 221 | +++ | +++ | ** | ** | 45 |
| 23 | 740 | 191 | 249 | ++ | + | * | * | 45 |
| 24 | 740 | 191 | 236 | ++ | ++ | * | * | 45 |
| 25 | 740 | 191 | 244 | ++ | ++ | * | ** | 45 |
| 26 | 740 | 191 | 220 | ++ | ++ | * | * | 45 |
| 27 | 740 | 191 | 221 | +++ | +++ | * | * | 45 |
| 28 | 740 | 195 | 249 | ++ | + | * | ND | 45 |
| 29 | 740 | 195 | 236 | ++ | ++ | * | * | 45 |
| 30 | 740 | 195 | 244 | ++ | ++ | * | * | 45 |
| 31 | 740 | 195 | 220 | + | + | ND | * | 45 |
| 32 | 740 | 195 | 221 | + | + | * | ND | 45 |
| 33 | 740 | 196 | 249 | + | + | ND | ND | 45 |
| 34 | 740 | 196 | 236 | + | + | * | ND | 45 |
| 35 | 740 | 196 | 244 | + | + | * | * | 45 |
| 36 | 740 | 196 | 220 | + | + | ND | ND | 45 |
| 37 | 740 | 196 | 221 | + | ++ | * | * | 45 |
| 38 | 740 | 202 | 249 | + | + | * | * | 45 |
| 39 | 740 | 202 | 249 | + | + | ND | * | 45 |
| 40 | 740 | 202 | 236 | + | + | * | ND | 45 |
| 41 | 740 | 202 | 244 | + | + | * | ND | 45 |
| 42 | 740 | 202 | 220 | + | + | ND | ND | 45 |
| 43 | 740 | 202 | 221 | ++ | ++ | * | * | 45 |
| 44 | 740 | 262 | 304 | +++ | +++ | ** | * | 50 |
| 45 | 740 | 262 | 304 | + | + | ND | ND | 51 |
| 46 | 740 | 263 | 305 | + | + | * | * | 50 |
| 47 | 740 | 263 | 320 | +++ | +++ | * | * | 50 |
| 48 | 740 | 263 | 309 | +++ | +++ | ** | ** | 50 |
| 49 | 740 | 263 | 305 | + | + | ND | ND | 51 |
| 50 | 740 | 263 | 320 | + | + | ND | ND | 51 |
| 51 | 740 | 263 | 309 | + | + | ND | ND | 51 |
| 52 | 740 | 264 | 305 | + | + | * | ND | 50 |
| 53 | 740 | 264 | 320 | ++ | +++ | * | * | 50 |
| 54 | 740 | 264 | 309 | +++ | +++ | * | ** | 50 |
| 55 | 740 | 264 | 305 | + | + | ND | ND | 51 |
| 56 | 740 | 264 | 320 | + | + | ND | ND | 51 |
| 57 | 740 | 264 | 309 | + | + | ND | ND | 51 |
| 58 | 740 | 268 | 304 | +++ | +++ | ** | ** | 50 |
| 59 | 740 | 268 | 304 | + | + | ND | ND | 51 |
| 60 | 740 | 271 | 305 | + | + | * | ND | 50 |
| 61 | 740 | 271 | 320 | +++ | +++ | * | * | 50 |
| 62 | 740 | 271 | 309 | +++ | +++ | ** | * | 50 |
| 63 | 740 | 271 | 305 | + | + | ND | ND | 51 |
| 64 | 740 | 271 | 320 | + | + | ND | ND | 51 |
| 65 | 740 | 271 | 309 | ND | + | ND | ND | 51 |
| 66 | 740 | 274 | 305 | + | + | * | ND | 50 |
| 67 | 740 | 274 | 320 | + | + | * | * | 50 |
| 68 | 740 | 274 | 309 | +++ | +++ | ** | ** | 50 |
| 69 | 740 | 274 | 305 | + | + | ND | ND | 51 |
| 70 | 740 | 274 | 320 | + | + | ND | ND | 51 |
| 71 | 740 | 27 | 309 | + | + | ND | ND | 51 |
| 72 | 740 | 275 | 304 | + | + | ND | ND | 51 |
| 73 | 740 | 283 | 305 | ++ | ++ | * | * | 50 |
| 74 | 740 | 283 | 320 | +++ | +++ | * | * | 50 |
| 75 | 740 | 283 | 309 | +++ | +++ | ** | ** | 50 |
| 76 | 740 | 283 | 305 | + | + | ND | ND | 51 |
| 77 | 740 | 283 | 320 | + | + | ND | ND | 51 |
| 78 | 740 | 283 | 309 | + | + | * | ND | 51 |
| 79 | 740 | 288 | 305 | + | + | * | * | 50 |
| 80 | 740 | 288 | 320 | +++ | +++ | * | *** | 50 |
| 81 | 740 | 288 | 309 | ++ | ++ | * | * | 50 |
| 82 | 740 | 288 | 305 | + | + | ND | ND | 51 |
| 83 | 740 | 288 | 320 | + | + | ND | ND | 51 |
| 84 | 740 | 288 | 309 | + | + | ND | ND | 51 |
| 85 | 740 | 290 | 305 | + | + | * | * | 50 |
| 86 | 740 | 290 | 320 | +++ | +++ | ** | ** | 50 |
| 87 | 740 | 290 | 309 | +++ | +++ | ** | * | 50 |
| 88 | 740 | 290 | 305 | + | + | ND | ND | 51 |
| 89 | 740 | 290 | 320 | + | + | * | * | 51 |
| 90 | 740 | 290 | 309 | + | + | ND | ND | 51 |
| 91 | 740 | 292 | 305 | + | + | ND | * | 50 |
| 92 | 740 | 292 | 320 | +++ | +++ | * | * | 50 |
| 93 | 740 | 292 | 309 | +++ | +++ | ** | ** | 50 |
| 94 | 740 | 292 | 305 | + | + | ND | ND | 51 |
| 95 | 740 | 292 | 320 | + | + | ND | ND | 51 |
| 96 | 740 | 292 | 309 | + | + | ND | ND | 51 |
| 97 | 740 | 338 | 410 | + | + | * | ND | 51 |
| 98 | 740 | 338 | 404 | + | + | ND | ND | 51 |
| 99 | 740 | 338 | 406 | ND | + | ND | ND | 51 |
| 100 | 740 | 338 | 414 | ++ | + | ND | ND | 51 |
| 101 | 740 | 338 | 418 | + | + | ND | ND | 51 |
| 102 | 740 | 338 | 419 | + | + | ND | ND | 51 |
| 103 | 740 | 338 | 417 | + | + | ND | ND | 51 |
| 104 | 740 | 338 | 412 | ND | + | ND | ND | 51 |
| 105 | 740 | 338 | 407 | + | + | ND | ND | 51 |
| 106 | 740 | 340 | 410 | +++ | +++ | * | * | 51 |
| 107 | 740 | 340 | 404 | +++ | +++ | * | * | 51 |
| 108 | 740 | 340 | 406 | +++ | +++ | * | * | 51 |
| 109 | 740 | 340 | 414 | ND | +++ | ND | * | 51 |
| 110 | 740 | 340 | 418 | +++ | +++ | * | * | 51 |
| 111 | 740 | 340 | 419 | +++ | +++ | ** | ** | 51 |
| 112 | 740 | 340 | 417 | +++ | +++ | * | * | 51 |
| 113 | 740 | 340 | 412 | ND | +++ | ND | *** | 51 |
| 114 | 740 | 340 | 407 | + | + | ND | * | 51 |
| 115 | 740 | 340 | 415 | ND | + | ND | * | 51 |
| 116 | 740 | 340 | 401 | + | + | * | ND | 51 |
| 117 | 740 | 340 | 399 | + | + | * | * | 51 |
| 118 | 740 | 342 | 416 | + | + | ND | ND | 51 |
| 119 | 740 | 342 | 413 | + | + | ND | ND | 51 |
| 120 | 740 | 351 | 410 | + | + | * | * | 51 |
| 121 | 740 | 351 | 404 | + | + | * | ND | 51 |
| 122 | 740 | 351 | 406 | + | + | * | ND | 51 |
| 123 | 740 | 351 | 414 | + | + | * | ND | 51 |
| 124 | 740 | 351 | 418 | + | + | * | * | 51 |
| 125 | 740 | 351 | 419 | + | + | * | * | 51 |
| 126 | 740 | 351 | 417 | + | + | ND | * | 51 |
| 127 | 740 | 351 | 412 | + | + | ** | ** | 51 |
| 128 | 740 | 351 | 407 | + | + | ND | ND | 51 |
| 129 | 740 | 351 | 415 | + | + | * | * | 51 |
| 130 | 740 | 351 | 401 | ++ | ++ | * | * | 51 |
| 131 | 740 | 351 | 399 | + | + | * | * | 51 |
| 132 | 740 | 352 | 409 | + | + | ND | ND | 51 |
| 133 | 740 | 352 | 422 | + | + | ND | ND | 51 |
| 134 | 740 | 352 | 421 | + | + | ND | ND | 51 |
| 135 | 740 | 352 | 420 | + | + | ND | ND | 51 |
| 136 | 740 | 352 | 424 | + | + | ND | ND | 51 |
| 137 | 740 | 352 | 416 | + | + | ND | ND | 51 |
| 138 | 740 | 352 | 413 | + | + | ND | ND | 51 |
| 139 | 740 | 357 | 410 | + | + | ND | ND | 51 |
| 140 | 740 | 357 | 404 | + | + | ND | ND | 51 |
| 141 | 740 | 357 | 406 | + | + | ND | ND | 51 |
| 142 | 740 | 357 | 414 | + | + | ND | ND | 51 |
| 143 | 740 | 357 | 418 | + | + | ND | ND | 51 |
| 144 | 740 | 357 | 419 | + | + | ND | ND | 51 |
| 145 | 740 | 357 | 417 | + | + | ND | ND | 51 |
| 146 | 740 | 357 | 412 | + | + | ND | ND | 51 |
| 147 | 740 | 357 | 407 | + | + | ND | ND | 51 |
| 148 | 740 | 357 | 415 | + | + | * | * | 51 |
| 149 | 740 | 357 | 401 | + | + | * | * | 51 |
| 150 | 740 | 357 | 399 | + | + | * | * | 51 |
| 151 | 740 | 360 | 410 | + | + | ND | ND | 51 |
| 152 | 740 | 360 | 404 | + | + | ND | ND | 51 |
| 153 | 740 | 360 | 406 | + | + | ND | ND | 51 |
| 154 | 740 | 360 | 414 | + | + | ND | ND | 51 |
| 155 | 740 | 360 | 418 | + | + | ND | ND | 51 |
| 156 | 740 | 360 | 419 | + | + | ND | ND | 51 |
| 157 | 740 | 360 | 417 | + | + | ND | ND | 51 |
| 158 | 740 | 360 | 412 | + | + | ND | ND | 51 |
| 159 | 740 | 360 | 407 | + | + | ND | ND | 51 |
| 160 | 740 | 360 | 415 | + | + | ND | ND | 51 |
| 161 | 740 | 360 | 401 | + | + | ND | ND | 51 |
| 162 | 740 | 360 | 399 | + | + | ND | ND | 51 |
| 163 | 740 | 376 | 410 | ++ | ++ | * | ND | 51 |
| 164 | 740 | 376 | 404 | + | + | * | * | 51 |
| 165 | 740 | 376 | 406 | ++ | ++ | * | * | 51 |
| 166 | 740 | 376 | 414 | ++ | ++ | * | ND | 51 |
| 167 | 740 | 376 | 418 | ++ | ++ | * | * | 51 |
| 168 | 740 | 376 | 419 | ++ | ++ | * | * | 51 |
| 169 | 740 | 376 | 417 | ++ | ++ | * | * | 51 |
| 170 | 740 | 376 | 412 | ++ | ++ | ** | ** | 51 |
| 171 | 740 | 376 | 407 | + | + | * | * | 51 |
| 172 | 740 | 376 | 415 | +++ | +++ | * | * | 51 |
| 173 | 740 | 376 | 401 | +++ | +++ | ** | ** | 51 |
| 174 | 740 | 376 | 399 | ++ | ++ | * | * | 51 |
| 175 | 740 | 373 | 409 | + | + | ND | ND | 51 |
| 176 | 740 | 373 | 422 | + | + | ND | ND | 51 |
| 177 | 740 | 373 | 421 | + | + | ND | ND | 51 |
| 178 | 740 | 373 | 420 | + | + | ND | ND | 51 |
| 179 | 740 | 373 | 424 | + | + | ND | ND | 51 |
| 180 | 740 | 373 | 416 | + | + | ND | ND | 51 |
| 181 | 740 | 373 | 413 | + | + | ND | ND | 51 |
| 182 | 740 | 375 | 409 | + | + | ND | ND | 51 |
| 183 | 740 | 375 | 422 | + | + | ND | ND | 51 |
| 184 | 740 | 375 | 421 | + | + | ND | ND | 51 |
| 185 | 740 | 375 | 420 | + | + | ND | ND | 51 |
| 186 | 740 | 375 | 424 | + | + | ND | ND | 51 |
| 187 | 740 | 375 | 416 | + | + | ND | ND | 51 |
| 188 | 740 | 375 | 413 | + | + | ND | ND | 51 |
| 189 | 740 | 430 | 467 | + | + | * | * | 53 |
| 190 | 740 | 430 | 495 | + | + | * | * | 53 |
| 191 | 740 | 430 | 494 | + | + | ND | ND | 53 |
| 192 | 740 | 437 | 467 | + | + | * | ND | 53 |
| 193 | 740 | 437 | 495 | + | + | ND | ND | 53 |
| 194 | 740 | 437 | 494 | + | + | ND | ND | 53 |
| 195 | 740 | 438 | 467 | + | + | * | * | 53 |
| 196 | 740 | 438 | 495 | + | + | ND | ND | 53 |
| 197 | 740 | 438 | 494 | + | + | * | * | 53 |
| 198 | 740 | 445 | 467 | + | + | * | * | 53 |
| 199 | 740 | 445 | 495 | + | + | * | * | 53 |
| 200 | 740 | 445 | 494 | + | + | * | * | 53 |
| 201 | 740 | 446 | 489 | + | + | ND | * | 53 |
| 202 | 740 | 446 | 490 | + | + | ND | ND | 53 |
| 203 | 740 | 446 | 485 | + | + | ND | * | 53 |
| 204 | 740 | 446 | 464 | +++ | ++ | ** | * | 53 |
| 205 | 740 | 446 | 466 | ++ | ++ | ** | * | 53 |
| 206 | 740 | 454 | 489 | + | + | ND | ND | 53 |
| 207 | 740 | 454 | 490 | + | + | * | ND | 53 |
| 208 | 740 | 454 | 485 | + | + | ND | * | 53 |
| 209 | 740 | 454 | 464 | ++ | ++ | * | * | 53 |
| 210 | 740 | 454 | 466 | +++ | ++ | * | * | 53 |
| 211 | 740 | 456 | 489 | + | ND | * | ND | 53 |
| 212 | 740 | 456 | 490 | + | + | ND | * | 53 |
| 213 | 740 | 456 | 485 | + | + | * | * | 53 |
| 214 | 740 | 456 | 464 | +++ | ++ | ** | * | 53 |
| 215 | 740 | 456 | 466 | ++ | ++ | * | * | 53 |
| 216 | 740 | 457 | 489 | + | + | * | * | 53 |
| 217 | 740 | 457 | 490 | + | + | * | ND | 53 |
| 218 | 740 | 457 | 485 | + | + | * | ND | 53 |
| 219 | 740 | 457 | 464 | +++ | ++ | ** | ** | 53 |
| 220 | 740 | 457 | 466 | ++ | ++ | ** | ** | 53 |
| 221 | 740 | 459 | 489 | + | + | * | * | 53 |
| 222 | 740 | 459 | 490 | + | + | * | * | 53 |
| 223 | 740 | 459 | 485 | + | + | * | ND | 53 |
| 224 | 740 | 459 | 464 | ++ | ++ | * | * | 53 |
| 225 | 740 | 459 | 466 | ++ | ++ | * | * | 53 |
| 226 | 740 | 460 | 489 | + | + | * | ND | 53 |
| 227 | 740 | 460 | 490 | + | + | ND | * | 53 |
| 228 | 740 | 460 | 485 | + | + | * | ND | 53 |
| 229 | 740 | 460 | 464 | ++ | + | * | * | 53 |
| 230 | 740 | 460 | 466 | + | + | * | * | 53 |
| 231 | 740 | 461 | 489 | + | + | ND | * | 53 |
| 232 | 740 | 461 | 490 | + | + | ND | ND | 53 |
| 233 | 740 | 461 | 485 | + | + | * | ND | 53 |
| 234 | 740 | 461 | 464 | ++ | ++ | ** | ** | 53 |
| 235 | 740 | 461 | 466 | + | ++ | * | * | 53 |
| 236 | 740 | 462 | 489 | + | + | * | * | 53 |
| 237 | 740 | 462 | 490 | + | + | ND | ND | 53 |
| 238 | 740 | 462 | 485 | + | + | ND | ND | 53 |
| 239 | 740 | 462 | 464 | ++ | ++ | * | * | 53 |
| 240 | 740 | 462 | 466 | ++ | ++ | * | * | 53 |
| 241 | 740 | 113 | 166 | +++ | +++ | *** | *** | 44 |
| 242 | 740 | 113 | 150 | +++ | +++ | *** | ND | 44 |
| 243 | 740 | 114 | 166 | +++ | +++ | *** | ND | 44 |
| 244 | 740 | 114 | 150 | +++ | ND | ND | ND | 44 |
| 245 | 740 | 118 | 158 | +++ | +++ | *** | *** | 44 |
| 246 | 740 | 118 | 175 | +++ | +++ | *** | *** | 44 |
| 247 | 740 | 118 | 178 | +++ | +++ | *** | *** | 44 |
| 248 | 740 | 118 | 162 | +++ | +++ | *** | *** | 44 |
| 249 | 740 | 118 | 176 | +++ | +++ | *** | *** | 44 |
| 250 | 740 | 118 | 171 | +++ | +++ | *** | *** | 44 |
| 251 | 740 | 118 | 170 | +++ | +++ | *** | *** | 44 |
| 252 | 740 | 118 | 156 | +++ | +++ | *** | *** | 44 |
| 253 | 740 | 118 | 160 | +++ | +++ | *** | *** | 44 |

“+” indicates <5% indel; “++” indicates ≥5% to <10% indel; “+++” indicates ≥10% indel; ND = Not detected; “*” indicates <1% exon deletion; “**” indicates ≥1 to <5; “***” indicates ≥5% exon deletion.

The data was further confirmed for exon deletion by sequencing. TABLE 23 provides sequences of primers that were used to confirm exon deletion.

TABLE 23
Sequencing Primers for Exon Deletion Confirmation

| | SEQ | | |
|---|---|---|---|
| | ID | Primer | |
| Exon | NO: | Type | Primer Sequence (5′ to 3′) |
| 44 | 1470 | Forward | TCGTCGGCAGCGTCAGATGTGTATAA |
| | | Primer | GAGACAGCTCTGCAAATGCAGGAAAC |
| | | | TATCAGAG |
| | 1471 | Reverse | GTCTCGTGGGCTCGGAGATGTGTATA |
| | | Primer | AGAGACAGTAAACCAGCTCCGTCCAG |
| | | | GC |
| 45 | 1472 | Forward | TCGTCGGCAGCGTCAGATGTGTATAA |
| | | Primer | GAGACAGCTGTCTTGTATCCTTTGGA |
| | | | TATGGGC |
| | 1473 | Reverse | GTCTCGTGGGCTCGGAGATGTGTATA |
| | | Primer | AGAGACAGCTGCTGTTGATTAATGGT |
| | | | TGATAGGTTC |
| 50 | 1474 | Forward | TCGTCGGCAGCGTCAGATGTGTATAA |
| | | Primer | GAGACAGGCAATTGATAAATATTTGT |
| | | | AGGGTGGTTGG |
| | 1475 | Reverse | GTCTCGTGGGCTCGGAGATGTGTATA |
| | | Primer | AGAGACAGAGTCAATTTCCAAGGAAT |
| | | | GTACTCTAAGAC |
| 51 | 1476 | Forward | TCGTCGGCAGCGTCAGATGTGTATAA |
| | | Primer | GAGACAGATTGAAATTGGCTCTTTAG |
| | | | CTTGTGTTTCT |
| | 1477 | Reverse | GTCTCGTGGGCTCGGAGATGTGTATA |
| | | Primer | AGAGACAGCAGTTGCCTAAGAACTGG |
| | | | TGGGA |
| 53 | 1478 | Forward | TCGTCGGCAGCGTCAGATGTGTATAA |
| | | Primer | GAGACAGTTGTTCATCATCCTAGCCA |
| | | | TAACACAAT |
| | 1479 | Reverse | GTCTCGTGGGCTCGGAGATGTGTATA |
| | | Primer | AGAGACAGGTCTACTGTTCATTTCAG |
| | | | CTTTAACGTG |

An analysis of sequencing data confirmed that CasPhi.12 can be used to delete a whole exon of interest to correct the frame of DMD in patients.

### Example 17. CasPhi.12 Modification of DMD in Primary Skeletal Myoblasts

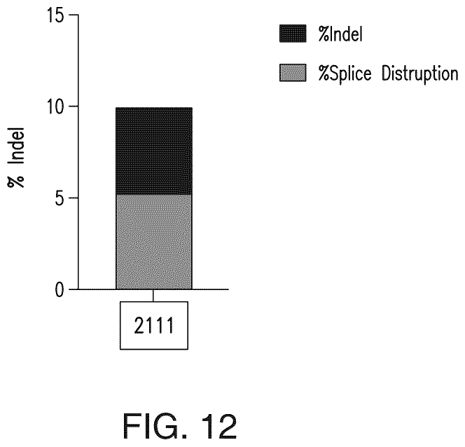

An RNP complex of CasPhi.12 L26R effector protein (L26R variant relative to SEQ ID NO: 2) and a guide nucleic acid were introduced to primary skeletal myoblasts using a P5 Nucleofector™ kit from Lonza. The guide nucleic acid comprised a nucleotide sequence of AUAGAUUGCUCCUUACGAGGAGACUGGUAUCUUAmCmAmG (SEQ ID NO: 1343), wherein “m” represents a 2′-O-Me modification of the following nucleotide, wherein the bold font sequence represents a repeat sequence, and wherein the non-bold font sequence represents a spacer sequence that is complementary to a target sequence within the human DMD gene. Primary skeletal myoblast cells were incubated for 72 hours after nucleofection before proceeding with library preparation and sequencing. Approximately 10% indel was achieved with nearly half of those indels predicted to result in a disruption of DMD gene splicing, as shown in FIG. 12. The disruption of DMD gene splicing at this site may result in skipping of DMD exon 45.

### Example 18: Indel Activity of CasM, 19952 Effector Protein (SEQ ID NO: 3)

Combinations of the effector protein (as set forth in SEQ ID NO: 3) and guide nucleic acids as set forth in TABLE 24 and target various exons (loci) of the DMD gene, as represented in TABLE 9 were tested for their ability to produce indels in HEK293T cells. Sequences of targeted exons are as set forth in TABLE 9.3. Some indels are predicted to result in exon skipping.

Briefly, 300 ng of plasmids expressing the effector protein (as set forth in SEQ ID NO: 3) and transcribing targeting gRNA were delivered by lipofection to HEK293T cells in 96 well plates. TransIT-293 reagent was diluted with warmed up OPTIMEM and mixed with the plasmid DNA at the ratio of 2:1 lipid:DNA. Lipid:DNA mixture were incubated for 10 minutes at room temperature before adding 20 μL of the lipid:DNA optimem mixture to each well. Cells were incubated for 3 days before being lysed and subjected to PCR amplification. Each composition was assayed in two replicate batches. Indels were detected by next generation sequencing of PCR amplicons at the targeted loci and indel percentage was calculated as the fraction of sequencing reads containing insertions or deletions relative to the unedited DMD gene sequence, and are provided in TABLE 25.

TABLE 24
Effector Protein (SEQ ID NO: 3) and
Guide Nucleic Acid Combinations

| | | | Spacer | sgRNA | |
|---|---|---|---|---|---|
| | | Handle | Sequence | Sequence | |
| | | SEQ | SEQ | SEQ | Exon |
| Name | PAM | ID NO: | ID NO: | ID NO: | No. |
| T01 | TTCG | 743 | 622 | 1344 | 44 |
| T02 | TTCG | 743 | 623 | 1345 | 44 |
| T03 | ATCG | 743 | 624 | 1346 | 44 |
| T04 | GTCG | 743 | 625 | 1347 | 45 |
| T05 | ATCG | 743 | 626 | 1348 | 50 |
| T06 | TTCG | 743 | 627 | 1349 | 50 |

TABLE 25
Indel activity of Effector Protein (SEQ ID NO: 3) and
Guide Nucleic Acid Combinations set forth in TABLE 24

| | | | % INDEL |
|---|---|---|---|
| Name | % INDEL REP1 | % INDEL REP2 | AVERAGE |
| T01 | 0.349972195 | 0.322459998 | 1.987985539 |
| T02 | 0.013492743 | 0.011561685 | 1.105562956 |
| T03 | 8.086218426 | 9.495194895 | 0.004546454 |
| T04 | 0.003006645 | 0.003214786 | 0.683736037 |
| T05 | 3.323898494 | 7.183384589 | 13.07060734 |
| T06 | 0.002975367 | 0.003115547 | 0.033993336 |

### Example 19: Indel Activity of Cas14a.1 Effector Protein (SEQ ID NO: 4)

Combinations of the effector protein (as set forth in SEQ ID NO: 4) and guide nucleic acids as set forth in TABLE 26 and target various exons (loci) of the DMD gene, as represented in TABLE 9 were tested for their ability to produce indels in HEK293T cells. Sequences of targeted exons are as set forth in TABLE 9.4. Some indels are predicted to result in exon skipping.

Briefly, 300 ng of plasmids expressing the effector protein (as set forth in SEQ ID NO: 4) and transcribing targeting gRNA were delivered by lipofection to HEK293T cells in 96 well plates. TransIT-293 reagent was diluted with warmed up OPTIMEM and mixed with the plasmid DNA at the ratio of 2:1 lipid:DNA. Lipid:DNA mixture were incubated for 10 minutes at room temperature before adding 20 μL of the lipid:DNA optimem mixture to each well. Cells were incubated for 3 days before being lysed and subjected to PCR amplification. Each composition was assayed in two replicate batches. Indels were detected by next generation sequencing of PCR amplicons at the targeted loci and indel percentage was calculated as the fraction of sequencing reads containing insertions or deletions relative to the unedited DMD gene sequence, and are provided in TABLE 27.

TABLE 26
Effector Protein (SEQ ID NO: 4) and
Guide Nucleic Acid, Combinations

| | Effector | | Handle | Spacer | sgRNA | |
|---|---|---|---|---|---|---|
| | Protein | | Sequence | Sequence | Sequence | |
| | SEQ | | SEQ | SEQ | SEQ | |
| Name | ID NO: | PAM | ID NO: | ID NO: | ID NO: | Exon |
| T01_Cas14a.1 | 4 | TTTA | 744 | 628 | 1350 | 53 |
| T02_Cas14a.1 | 4 | TTTA | 744 | 629 | 1351 | 53 |
| T03_Cas14a.1 | 4 | TTTA | 744 | 630 | 1352 | 53 |
| T04_Cas14a.1 | 4 | TTTA | 744 | 631 | 1353 | 53 |
| T05_Cas14a.1 | 4 | TTTG | 744 | 632 | 1354 | 53 |
| T06_Cas14a.1 | 4 | TTTA | 744 | 633 | 1355 | 53 |
| T07_Cas14a.1 | 4 | TTTA | 744 | 634 | 1356 | 53 |
| T08_Cas14a.1 | 4 | TTTA | 744 | 635 | 1357 | 53 |
| T09_Cas14a.1 | 4 | TTTA | 744 | 636 | 1358 | 53 |
| T10_Cas14a.1 | 4 | TTTG | 744 | 637 | 1359 | 53 |
| T11_Cas14a.1 | 4 | TTTA | 744 | 638 | 1360 | 53 |
| T12_Cas14a.1 | 4 | TTTA | 744 | 639 | 1361 | 53 |
| T13_Cas14a.1 | 4 | TTTG | 744 | 640 | 1362 | 53 |
| T14_Cas14a.1 | 4 | TTTG | 744 | 641 | 1363 | 53 |
| T15_Cas14a.1 | 4 | TTTA | 744 | 642 | 1364 | 53 |
| T16_Cas14a.1 | 4 | TTTA | 744 | 643 | 1365 | 53 |
| T17_Cas14a.1 | 4 | TTTA | 744 | 644 | 1366 | 53 |
| T18_Cas14a.1 | 4 | TTTG | 744 | 645 | 1367 | 53 |
| T19_Cas14a.1 | 4 | TTTA | 744 | 646 | 1368 | 53 |
| T20_Cas14a.1 | 4 | TTTA | 744 | 647 | 1369 | 53 |
| T21_Cas14a.1 | 4 | TTTG | 744 | 648 | 1370 | 53 |
| T22_Cas14a.1 | 4 | TTTA | 744 | 649 | 1371 | 53 |
| T23_Cas14a.1 | 4 | TTTA | 744 | 650 | 1372 | 53 |
| T24_Cas14a.1 | 4 | TTTA | 744 | 651 | 1373 | 53 |
| T25_Cas14a.1 | 4 | TTTA | 744 | 652 | 1374 | 53 |
| T26_Cas14a.1 | 4 | TTTG | 744 | 653 | 1375 | 51 |
| T27_Cas14a.1 | 4 | TTTA | 744 | 654 | 1376 | 51 |
| T28_Cas14a.1 | 4 | TTTG | 744 | 655 | 1377 | 51 |
| T29_Cas14a.1 | 4 | TTTA | 744 | 656 | 1378 | 51 |
| T30_Cas14a.1 | 4 | TTTA | 744 | 657 | 1379 | 51 |
| T31_Cas14a.1 | 4 | TTTG | 744 | 658 | 1380 | 51 |
| T32_Cas14a.1 | 4 | TTTG | 744 | 659 | 1381 | 51 |
| T33_Cas14a.1 | 4 | TTTA | 744 | 660 | 1382 | 51 |
| T34_Cas14a.1 | 4 | TTTA | 744 | 661 | 1383 | 51 |
| T35_Cas14a.1 | 4 | TTTG | 744 | 662 | 1384 | 51 |
| T36_Cas14a.1 | 4 | TTTG | 744 | 663 | 1385 | 51 |
| T37_Cas14a.1 | 4 | TTTA | 744 | 664 | 1386 | 51 |
| T38_Cas14a.1 | 4 | TTTG | 744 | 665 | 1387 | 51 |
| T39_Cas14a.1 | 4 | TTTA | 744 | 666 | 1388 | 51 |
| T40_Cas14a.1 | 4 | TTTG | 744 | 667 | 1389 | 51 |
| T41_Cas14a.1 | 4 | TTTA | 744 | 668 | 1390 | 51 |
| T42_Cas14a.1 | 4 | TTTG | 744 | 669 | 1391 | 51 |
| T43_Cas14a.1 | 4 | TTTG | 744 | 670 | 1392 | 51 |
| T44_Cas14a.1 | 4 | TTTA | 744 | 671 | 1393 | 51 |
| T45_Cas14a.1 | 4 | TTTG | 744 | 672 | 1394 | 51 |
| T46_Cas14a.1 | 4 | TTTG | 744 | 673 | 1395 | 50 |
| T47_Cas14a.1 | 4 | TTTG | 744 | 674 | 1396 | 50 |
| T48_Cas14a.1 | 4 | TTTG | 744 | 675 | 1397 | 50 |
| T49_Cas14a.1 | 4 | TTTA | 744 | 676 | 1398 | 50 |
| T50_Cas14a.1 | 4 | TTTA | 744 | 677 | 1399 | 50 |
| T51_Cas14a.1 | 4 | TTTG | 744 | 678 | 1400 | 50 |
| T52_Cas14a.1 | 4 | TTTG | 744 | 679 | 1401 | 50 |
| T53_Cas14a.1 | 4 | TTTG | 744 | 680 | 1402 | 50 |
| T54_Cas14a.1 | 4 | TTTG | 744 | 681 | 1403 | 50 |
| T55_Cas14a.1 | 4 | TTTA | 744 | 682 | 1404 | 50 |
| T56_Cas14a.1 | 4 | TTTG | 744 | 683 | 1405 | 50 |
| T57_Cas14a.1 | 4 | TTTA | 744 | 684 | 1406 | 50 |
| T58_Cas14a.1 | 4 | TTTA | 744 | 685 | 1407 | 50 |
| T59_Cas14a.1 | 4 | TTTG | 744 | 686 | 1408 | 50 |
| T60_Cas14a.1 | 4 | TTTA | 744 | 687 | 1409 | 50 |
| T61_Cas14a.1 | 4 | TTTA | 744 | 688 | 1410 | 50 |
| T62_Cas14a.1 | 4 | TTTA | 744 | 689 | 1411 | 50 |
| T63_Cas14a.1 | 4 | TTTG | 744 | 690 | 1412 | 50 |
| T64_Cas14a.1 | 4 | TTTA | 744 | 691 | 1413 | 50 |
| T65_Cas14a.1 | 4 | TTTA | 744 | 692 | 1414 | 45 |
| T66_Cas14a.1 | 4 | TTTA | 744 | 693 | 1415 | 45 |
| T67_Cas14a.1 | 4 | TTTA | 744 | 694 | 1416 | 45 |
| T68_Cas14a.1 | 4 | TTTA | 744 | 695 | 1417 | 45 |
| T69_Cas14a.1 | 4 | TTTA | 744 | 696 | 1418 | 45 |
| T70_Cas14a.1 | 4 | TTTA | 744 | 697 | 1419 | 45 |
| T71_Cas14a.1 | 4 | TTTA | 744 | 698 | 1420 | 45 |
| T72_Cas14a.1 | 4 | TTTG | 744 | 699 | 1421 | 45 |
| T73_Cas14a.1 | 4 | TTTA | 744 | 700 | 1422 | 45 |
| T74_Cas14a.1 | 4 | TTTG | 744 | 701 | 1423 | 45 |
| T75_Cas14a.1 | 4 | TTTG | 744 | 702 | 1424 | 45 |
| T76_Cas14a.1 | 4 | TTTG | 744 | 703 | 1425 | 45 |
| T77_Cas14a.1 | 4 | TTTG | 744 | 704 | 1426 | 45 |
| T78_Cas14a.1 | 4 | TTTG | 744 | 705 | 1427 | 45 |
| T79_Cas14a.1 | 4 | TTTG | 744 | 706 | 1428 | 45 |
| T80_Cas14a.1 | 4 | TTTA | 744 | 707 | 1429 | 45 |
| T81_Cas14a.1 | 4 | TTTA | 744 | 708 | 1430 | 44 |
| T82_Cas14a.1 | 4 | TTTG | 744 | 709 | 1431 | 44 |
| T83_Cas14a.1 | 4 | TTTG | 744 | 710 | 1432 | 44 |
| T84_Cas14a.1 | 4 | TTTG | 744 | 711 | 1433 | 44 |
| T85_Cas14a.1 | 4 | TTTA | 744 | 712 | 1434 | 44 |
| T86_Cas14a.1 | 4 | TTTA | 744 | 713 | 1435 | 44 |
| T87_Cas14a.1 | 4 | TTTG | 744 | 714 | 1436 | 44 |
| T88_Cas14a.1 | 4 | TTTA | 744 | 715 | 1437 | 44 |
| T89_Cas14a.1 | 4 | TTTG | 744 | 716 | 1438 | 44 |
| T90_Cas14a.1 | 4 | TTTA | 744 | 717 | 1439 | 44 |
| T91_Cas14a.1 | 4 | TTTG | 744 | 718 | 1440 | 44 |
| T92_Cas14a.1 | 4 | TTTG | 744 | 719 | 1441 | 44 |
| T93_Cas14a.1 | 4 | TTTA | 744 | 720 | 1442 | 44 |
| T94_Cas14a.1 | 4 | TTTA | 744 | 721 | 1443 | 44 |
| T95_Cas14a.1 | 4 | TTTA | 744 | 722 | 1444 | 44 |
| T96_Cas14a.1 | 4 | TTTG | 744 | 723 | 1445 | 44 |

TABLE 27
Indel activity of Cas14a.1 Effector Protein (SEQ ID NO:
4) and Guide Nucleic Acid Combinations of TABLE 26

| NAME | % INDEL REP1 | % INDEL REP2 | % INDEL AVERAGE |
|---|---|---|---|
| T01 | 0 | 0 | 0 |
| T02 | 0.006294284789411215 | 0.004735227910161799 | 0.005514756 |
| T03 | 0.01565738231342094 | 0.018091623216618242 | 0.016874503 |
| T04 | 0.006049588476743869 | 0.004928576709189332 | 0.005489083 |
| T05 | 0.013996495044365974 | 0.014194296258620079 | 0.014095396 |
| T06 | 0.03780849276876499 | 0.05229793977812996 | 0.045053216 |
| T07 | 0.5940019882911742 | 0.46085424956734955 | 0.527428119 |
| T08 | 0.0009234207197141088 | 0.0012032680760946733 | 0.001063344 |
| T09 | 0.11150388606111016 | 0.23253482237887363 | 0.172019354 |
| T10 | 0 | 0 | 0 |
| T11 | 0.4109293919574868 | 0.38443990695656727 | 0.39768465 |
| T12 | 0.186333547915699 | 0.17278519071070278 | 0.179559369 |
| T13 | 0.050151061074874013 | 0.12502673875681378 | 0.0875889 |
| T14 | 0.008785755361973121 | 0.007647492317746354 | 0.008216624 |
| T15 | 0.01926276488662352 | 0.02560554655531537 | 0.022434156 |
| T16 | 0.009307488391493646 | 0.008992522717360516 | 0.009150006 |
| T17 | 0.005012859913701689 | 0.003936011695577609 | 0.004474436 |
| T18 | 0.003948813504942187 | 0.005104471517048934 | 0.004526643 |
| T19 | 0.18285901942783786 | 0.21154966869570196 | 0.197204344 |
| T20 | 0.01636684649739256 | 0.0160353082215316 | 0.016201077 |
| T21 | 0.004798336576653427 | 0.004115974439798728 | 0.004457156 |
| T22 | 0.17836763684827978 | 0.17284238328284007 | 0.17560501 |
| T23 | 0.0077971728960209265 | 0.006094381657265518 | 0.006945777 |
| T24 | 0.0743261460435901 | 0.06292318738756823 | 0.068624667 |
| T25 | 0.05437727911526697 | 0.05788712011577424 | 0.0561322 |
| T26 | 0.022871288460762286 | 0.01530323860957736 | 0.019087264 |
| T27 | 0.04598328794878612 | 0.04070740422452395 | 0.043345346 |
| T28 | 0.04597831639893285 | 0.04132935662547933 | 0.043653837 |
| T29 | 0.016243860962702573 | 0.023406064235878054 | 0.019824963 |
| T30 | 0.0015370562299084794 | 0.0034732527959685008 | 0.002505155 |
| T31 | 0.04405191098811409 | 0.04680622975819298 | 0.04542907 |
| T32 | 15.237748359446298 | 11.411508574123532 | 13.32462847 |
| T33 | 10.000480078907515 | 6.482278097546199 | 8.241379088 |
| T34 | 0.2910819550717586 | 0.27705722601780475 | 0.284069591 |
| T35 | 0.0223453406678628 | 0.022026340178122442 | 0.02218584 |
| T36 | 0.1429978963065534 | 0.14233565012339297 | 0.142666773 |
| T37 | 0.01217599121063332 | 0.019043118557519036 | 0.015609555 |
| T38 | 0.04507038934335683 | 0.029489953900263238 | 0.037280172 |
| T39 | 0.04162982757321894 | 0.015269104809241494 | 0.028449466 |
| T40 | 0.03281925325611986 | 0.021931267407943506 | 0.02737526 |
| T41 | 0.0407874343124949 | 0.051245585530471253 | 0.04601651 |
| T42 | 0.007039444353192388 | 0.006871102165205548 | 0.006955273 |
| T43 | 0.04732209324509852 | 0.04972272110987634 | 0.048522407 |
| T44 | 0.014284141329675006 | 0.013694936725112662 | 0.013989539 |
| T45 | 0.003899816708614695 | 0.0037197306914979354 | 0.003809774 |
| T46 | 0.014237330829023346 | 0.017967165005951625 | 0.016102248 |
| T47 | 0.013032928285719702 | 0.01581118085972455 | 0.014422055 |
| T48 | 0.003251609546725629 | 0.0028372745656390576 | 0.003044442 |
| T49 | 0.00566164588763992 | 0.005139699898633696 | 0.005400673 |
| T50 | 0.030550105567587017 | 0.022010582010582008 | 0.026280344 |
| T51 | 0.003920745587431474 | 0.004867083005058725 | 0.004393914 |
| T52 | 0.019257442757860815 | 0.018088935185473963 | 0.018673189 |
| T53 | 0.003454258968451987 | 0.003265348771902327 | 0.003359804 |
| T54 | 0.011356224375698845 | 0.011039821284775436 | 0.011198023 |
| T55 | 0.006408267339422312 | 0.0040462895524803755 | 0.005227278 |
| T56 | 0.020985848238861533 | 0.015199750048554757 | 0.018092799 |
| T57 | 0.008025495185997322 | 0.010467168773580867 | 0.009246332 |
| T58 | 0.029348786367788208 | 0.03172816996146085 | 0.030538478 |
| T59 | 0.3137754344519488 | 0.27140447166751097 | 0.292589953 |
| T60 | 0.45789311542408306 | 0.3201036230896042 | 0.388998369 |
| T61 | 0.011513000095941667 | 0.00807372463985421 | 0.009793362 |
| T62 | 0.006576746340612552 | 0.005462604569858909 | 0.006019675 |
| T63 | 0.02135133475090597 | 0.030194649167946043 | 0.025772992 |
| T64 | 0.014686275243835364 | 0.010924094350562592 | 0.012805185 |
| T65 | 0.0029313009646644993 | 0.0021522115767460713 | 0.002541756 |
| T66 | 0.022010502153884854 | 0.01890833582579578 | 0.020459419 |
| T67 | 0.07577888059437586 | 0.07403867511156283 | 0.074908778 |
| T68 | 0.0032986154061832545 | 0.001090310411374118 | 0.002194463 |
| T69 | 0.025929516809091113 | 0.034374879301687845 | 0.030152198 |
| T70 | 0.008022261776429594 | 0.00910571737876749 | 0.00856399 |
| T71 | 0.03867523782088113 | 0.041074827152396215 | 0.039875032 |
| T72 | 0.010633292306547184 | 0.010264042493135922 | 0.010448667 |
| T73 | 0.0008237141821616456 | 0.00167065396078641 | 0.001247184 |
| T74 | 0.005390254420008625 | 0.004462393181463219 | 0.004926324 |
| T75 | 0.002418704649287826 | 0.0010964150881334995 | 0.00175756 |
| T76 | 0 | 0.12267277038499802 | 0.061336385 |
| T77 | 0.0744740768818051 | 0.055246255729571356 | 0.064860166 |
| T78 | 0.0012658502535317223 | 0.0018315144105843218 | 0.001548682 |
| T79 | 0.012175846795684696 | 0.013991384879182903 | 0.013083616 |
| T80 | 0.03672047769505013 | 0.038308531015176076 | 0.037514504 |
| T81 | 0.0022536057692307695 | 0.0025192851276521775 | 0.002386445 |
| T82 | 0.014344985875706215 | 0.01419560081970495 | 0.014270293 |
| T83 | 0.02471700072697061 | 0.019399742132658424 | 0.022058371 |
| T84 | 0.006310048340457287 | 0.007833446680933035 | 0.007071748 |
| T85 | 0.017630260951059788 | 0.02293720166139731 | 0.020283731 |
| T86 | 0.0246456310337786 | 0.024623006842211297 | 0.024634319 |
| T87 | 0.011205109529945655 | 0.013649703118957161 | 0.012427406 |
| T88 | 0.010935367603317352 | 0.012092048392997774 | 0.011513708 |
| T89 | 0.033552986215773205 | 0.004512928128798969 | 0.019032957 |
| T90 | 0.004026197122611123 | 0.0018400279684251202 | 0.002933113 |
| T91 | 0.010233832178216743 | 0 | 0.005116916 |
| T92 | 0.0046197319015586465 | 0.005931432638696666 | 0.005275582 |
| T93 | 0.0068803552098049645 | 0.01139641957058291 | 0.009138387 |
| T94 | 0.8989192792856016 | 0.5665523790101584 | 0.732735829 |
| T95 | 0.007321093724169528 | 0.00968101069751682 | 0.008501052 |
| T96 | 0.001910110213359311 | 0.0028748399236860674 | 0.002392475 |

### Example 20: Indel Activity of Effector Protein

Combinations of the effector protein (as set forth in SEQ ID NO: 4) and guide nucleic acids targeting the DMD gene were tested as described in Example 1 for their ability to produce indels in the DMD gene. Tested guide nucleic acids were all sgRNAs and each used a handle sequence of 5′-ACCGCTTCACCAAgtGCTGTCCCTTAGGGATTAGcACTTGAGTGAAGGTGGGCTGCTTGCATCA GCCTAATGTCGAGAAGTGCTTTCTTCGGAAAGTAACCCTCGAAACAAATTCATTTGAAAGAATG AAGGAATGCAAC 3′ (SEQ ID NO: 1632) or 5′-ACCGCUUCACCAAgUGCUGUCCCUUAGGGAUUAGcACUUGAGUGAAGGUGGGCUGCUUGC AUCAGCCUAAUGUCGAGAAGUGCUUUCUUCGGAAAGUAACCCUCGAAACAAAUUCAUUUG AAAGAAUGAAGGAAUGCAAC-3′ (SEQ ID NO: 746) with a sequence of TABLE 28 linked to the 3′ end.

The handle sequence used in this experiment is made up of different regions: the handle sequence without the linker and repeat sequence, the linker, and the repeat sequence. The handle sequence without the linker and repeat sequence is in italics, the linker is in bold and the repeat sequence is in plaintext. The nucleotides in lower case denotes the changes engineered to stabilize the thermostability of the effector protein and led to higher activity even at lower temperatures.

Indels were detected and calculated as set forth in Example 19, and indel activity of assayed combinations are provided in TABLE 29.

TABLE 28
Effector Protein (SEQ ID NO: 4) and
Guide Nucleic Acid* Combinations

| | Effector | | Handle | Spacer | |
|---|---|---|---|---|---|
| | Protein | | Sequence | sequence | SgRNA |
| | SEQ | | SEQ | SEQ | SEQ |
| Name | ID NO | PAM | ID NO | ID NO: | ID NO |
| T97_Cas14a.1 | 4 | TTTN | 746 | 724 | 1446 |
| T98_Cas14a.1 | 4 | TTTN | 746 | 725 | 1447 |
| T99_Cas14a.1 | 4 | TTTN | 746 | 726 | 1448 |
| T100_Cas14a.1 | 4 | TTTN | 746 | 727 | 1449 |
| T101_Cas14a.1 | 4 | TTTN | 746 | 728 | 1450 |
| T102_Cas14a.1 | 4 | TTTN | 746 | 729 | 1451 |
| T103_Cas14a.1 | 4 | TTTN | 746 | 730 | 1452 |
| T104_Cas14a.1 | 4 | TTTN | 746 | 731 | 1453 |
| T105_Cas14a.1 | 4 | TTTN | 746 | 732 | 1454 |
| T106_Cas14a.1 | 4 | TTTN | 746 | 733 | 1455 |
| T107_Cas14a.1 | 4 | TTTN | 746 | 734 | 1456 |
| T108_Cas14a.1 | 4 | TTTN | 746 | 735 | 1457 |
| T109_Cas14a.1 | 4 | TTTN | 746 | 736 | 1458 |
| T110_Cas14a.1 | 4 | TTTN | 746 | 737 | 1459 |
| T111_Cas14a.1 | 4 | TTTN | 746 | 738 | 1460 |

TABLE 29
Indel activity of Effector Protein (SEQ ID NO: 4)
and Guide Nucleic Acid Combinations of TABLE 28

| Name | % indel rep 1 | % indel rep 2 |
|---|---|---|
| T97 | 0.01842 | 0 |
| T98 | 0.276912 | 0.226851 |
| T99 | 0 | 0 |
| T100 | 2.855216 | 1.651685 |
| T101 | 0 | 0 |
| T102 | 0.042355 | 0 |
| T103 | 0.556407 | 0.290276 |
| T104 | 0.328037 | 0.325155 |
| T105 | 0.702948 | 0 |
| T106 | 0.008574 | 0.084926 |
| T107 | 0.01066 | 0 |
| T108 | 0.016661 | 0.0456 |
| T109 | 0.018345 | 0.016861 |
| T110 | 0 | 0 |
| T111 | 0.780937 | 1.408451 |

1. A composition that comprises:
(a) an effector protein or a nucleic acid encoding the effector protein, and
(b) a guide nucleic acid or a nucleic acid encoding the guide nucleic acid, wherein the guide nucleic acid comprises a spacer sequence that hybridizes to a target sequence in a target nucleic acid and is at least 90% identical to any one of the nucleotide sequences selected from TABLE 4.

2.-3. (canceled)

4. The composition of claim 1, wherein the guide nucleic acid comprises a nucleotide sequence that is at least 90% identical to any one of the nucleotide sequences selected from TABLE 7 and TABLE 8.

5.-9. (canceled)

10. The composition of claim 1, wherein the guide nucleic acid comprises a nucleotide sequence that interacts with the effector protein and is at least 90% identical to any one of the nucleotide sequences selected from TABLE 5, TABLE 5.1 and TABLE 6.

11.-12. (canceled)

13. The composition of claim 1, wherein the effector protein comprises an amino acid sequence that is at least 80%, at least 85%, at least 90%, at least 95%, at least 98%, or 100% identical to any one of the amino acid sequences selected from TABLE 1.

14.-21. (canceled)

22. The composition of claim 1, wherein the effector protein is about 400 to about 700 amino acids in length.

23. The composition of claim 1, wherein the target sequence is adjacent to a protospacer adjacent motif (PAM) selected from TABLE 3.

24.-26. (canceled)

27. The composition of claim 1, wherein the effector protein comprises at least one amino acid alteration relative to a corresponding sequence in TABLE 1 that reduces its nuclease activity, relative to an otherwise comparable effector protein without the mutation, as measured in a cleavage assay.

28. The composition of claim 1, wherein a fusion partner is fused to the effector protein, or the composition comprises a nucleic acid encoding a fusion partner fused to the effector protein.

29. (canceled)

30. The composition of claim 28, wherein the fusion partner is selected from a reverse transcriptase, a deaminase, a transcriptional activator, a transcriptional repressor, or a functional domain thereof.

31.-35. (canceled)

36. The composition of claim 1, comprising a nucleic acid expression vector, wherein the nucleic acid encoding the effector protein and the nucleic acid encoding the guide nucleic acid are located in the nucleic acid expression vector, optionally, wherein the nucleic acid expression vector comprises an adeno associated viral (AAV) vector.

37.-40. (canceled)

41. A pharmaceutical composition, comprising the composition of claim 1, and a pharmaceutically acceptable excipient, carrier or diluent.

42. A system comprising components comprising the composition of claim 1.

43. (canceled)

44. A method of modifying a target nucleic acid within a human dystrophin gene (DMD gene), wherein the method comprises contacting the DMD gene with the composition of claim 1, wherein the DMD gene is the target nucleic acid, thereby modifying the DMD gene.

45.-49. (canceled)

50. A cell contacted or modified by the composition of claim 1.

51.-53. (canceled)

54. The cell of claim 50, wherein the cell is contacted or modified ex vivo.

55. The cell of claim 50, wherein the cell is contacted or modified in vivo.

56. (canceled)

57. A method of treating a disease associated with a mutation of a human DMD gene in a subject in need thereof, the method comprising administering to the subject the composition of claim 1.

58. The method of claim 57, wherein the disease or disorder is any one of the diseases or disorders set forth in TABLE 10.

59.-60. (canceled)

61. A composition that comprises:
a) an effector protein or a nucleic acid encoding the effector protein, and
b) a guide nucleic acid or a nucleic acid encoding the guide nucleic acid, wherein the guide nucleic acid comprises a spacer sequence that hybridizes to a target sequence in DMD, wherein the target sequence is adjacent to a protospacer adjacent motif (PAM) selected from TABLE 3.

62. A guide nucleic acid or a nucleic acid encoding the guide nucleic acid, wherein the guide nucleic acid comprises a nucleotide sequence selected from TABLE 7 and TABLE 8.
